SEQUENCE LISTING (328 sequences)

SEQ ID NO: 1 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 216 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 4 - protein, 217 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 5 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 217 aa, Homo sapiens
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 217 aa, Homo sapiens
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 119 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSS

SEQ ID NO: 11 - protein, 5 aa, synthetic construct
RYWMN

SEQ ID NO: 12 - protein, 17 aa, synthetic construct
EITPDSSTINYTPSLKG

SEQ ID NO: 13 - protein, 10 aa, synthetic construct
PYDYGAWFAS

SEQ ID NO: 14 - protein, 30 aa, synthetic construct
EVQLVESGGGLVQPGGSLRLSCAASGFDFS

SEQ ID NO: 15 - protein, 14 aa, synthetic construct
WVRQAPGKGLEWVG

SEQ ID NO: 16 - protein, 32 aa, synthetic construct
RFTISRDNAKNSLYLQMNSLRAEDTAVYYCVR

SEQ ID NO: 17 - protein, 11 aa, synthetic construct
WGQGTLVTVSS

SEQ ID NO: 18 - protein, 119 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSS

SEQ ID NO: 19 - protein, 17 aa, synthetic construct
EITPDSSTINYAPSLKG

SEQ ID NO: 20 - protein, 119 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSS

SEQ ID NO: 21 - protein, 30 aa, synthetic construct
EVQLVESGGGLVQPGGSLRLSCAASGFTFS

SEQ ID NO: 22 - protein, 32 aa, synthetic construct
RFTISRDNAKNSLYLQMNSLRAEDTAVYYCAR

SEQ ID NO: 23 - protein, 109 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 24 - protein, 14 aa, synthetic construct
RSSTGAVTTSNYAN

SEQ ID NO: 25 - protein, 7 aa, synthetic construct
GTNKRAP

SEQ ID NO: 26 - protein, 9 aa, synthetic construct
ALWYSNHWV

SEQ ID NO: 27 - protein, 22 aa, synthetic construct
QAVVTQEPSLTVSPGGTVTLTC

SEQ ID NO: 28 - protein, 15 aa, synthetic construct
WVQEKPDHLFTGLIG

SEQ ID NO: 29 - protein, 32 aa, synthetic construct
GTPARFSGSLLGGKAALTLSGAQPEDEAEYYC

SEQ ID NO: 30 - protein, 10 aa, synthetic construct
FGGGTKLTVL

SEQ ID NO: 31 - protein, 5 aa, synthetic construct
GGGGS

SEQ ID NO: 32 - protein, 15 aa, synthetic construct
GGGGSGGGGSGGGGS

SEQ ID NO: 33 - protein, 243 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 34 - protein, 243 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 35 - protein, 243 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 36 - protein, 120 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 37 - protein, 111 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 38 - protein, 133 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 39 - protein, 133 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLT

SEQ ID NO: 40 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 41 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 43 - protein, 374 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 44 - protein, 374 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLT

SEQ ID NO: 45 - protein, 374 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 46 - protein, 374 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLT

SEQ ID NO: 47 - protein, 375 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 375 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLTGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 375 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 375 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 51 - protein, 375 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLT

SEQ ID NO: 52 - protein, 375 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 53 - protein, 375 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLTGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 54 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 55 - protein, 448 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 56 - protein, 218 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLT

SEQ ID NO: 58 - protein, 607 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSAPASSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFAQSIISTSPQ

SEQ ID NO: 59 - protein, 444 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 60 - protein, 218 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 61 - protein, 101 aa, synthetic construct
AKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 62 - protein, 368 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 63 - protein, 349 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 64 - protein, 629 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVGSGQCTNYALLKLAGDVESNPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 65 - protein, 86 aa, synthetic construct
EMETSQFPGEEKPQASPEGRPESETSCLVTTTDFQIQTEMAATMETSIFTTEYQVAVAGCVFLLISVLLLSGLTWQRRQRKSRRTI

SEQ ID NO: 66 - protein, 353 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 67 - protein, 334 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 68 - protein, 595 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSEMETSQFPGEEKPQASPEGRPESETSCLVTTTDFQIQTEMAATMETSIFTTEYQVAVAGCVFLLISVLLLSGLTWQRRQRKSRRTIGSGQCTNYALLKLAGDVESNPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 69 - protein, 117 aa, synthetic construct
QLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 70 - protein, 384 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 71 - protein, 365 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 72 - protein, 626 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLGSGQCTNYALLKLAGDVESNPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 73 - protein, 27 aa, Homo sapiens
FWVLVVVGGVLACYSLLVTVAFIIFWV

SEQ ID NO: 74 - protein, 41 aa, Homo sapiens
RSKRSRLLHSDYMNMTPRRPGPTRKHYQPYAPPRDFAAYRS

SEQ ID NO: 75 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 42 aa, Homo sapiens
KRGRKKLLYIFKQPFMRPVQTTQEEDGCSCRFPEEEEGGCEL

SEQ ID NO: 77 - protein, 20 aa, synthetic construct
GGGGSGGGGSGGGGSGGGGS

SEQ ID NO: 78 - protein, 5 aa, synthetic construct
GGGGS

SEQ ID NO: 79 - protein, 19 aa, synthetic construct
GEGRGSLLTCGDVEENPGP

SEQ ID NO: 80 - protein, 47 aa, Homo sapiens
KPTTTPAPRPPTPAPTIASQPLSLRPEACRPAAGGAVHTRGLDFACD

SEQ ID NO: 81 - DNA, 780 bp, Homo sapiens
  source 1..780: mol_type = other DNA
atggcgcgcccgcatccgtggtggctgtgcgtgctgggcaccctggtgggcctgagcgcgaccccggcgccgaaaagctgcccggaacgccattattgggcgcagggcaaactgtgctgccagatgtgcgaaccgggcacctttctggtgaaagattgcgatcagcatcgcaaagcggcgcagtgcgatccgtgcattccgggcgtgagctttagcccggatcatcatacccgcccgcattgcgaaagctgccgccattgcaacagcggcctgctggtgcgcaactgcaccattaccgcgaacgcggaatgcgcgtgccgcaacggctggcagtgccgcgataaagaatgcaccgaatgcgatccgctgccgaacccgagcctgaccgcgcgcagcagccaggcgctgagcccgcatccgcagccgacccatctgccgtatgtgagcgaaatgctggaagcgcgcaccgcgggccatatgcagaccctggcggattttcgccagctgccggcgcgcaccctgagcacccattggccgccgcagcgcagcctgtgcagcagcgattttattcgcattctggtgatttttagcggcatgtttctggtgtttaccctggcgggcgcgctgtttctgcatcagcgccgcaaatatcgcagcaacaaaggcgaaagcccggtggaaccggcggaaccgtgccattatagctgcccgcgcgaagaagaaggcagcaccattccgattcaggaagattatcgcaaaccggaaccggcgtgcagcccg

SEQ ID NO: 82 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 83 - DNA, 750 bp, Mus musculus
  source 1..750: mol_type = other DNA
atggcgtggccgccgccgtattggctgtgcatgctgggcaccctggtgggcctgagcgcgaccctggcgccgaacagctgcccggataaacattattggaccggcggcggcctgtgctgccgcatgtgcgaaccgggcaccttttttgtgaaagattgcgaacaggatcgcaccgcggcgcagtgcgatccgtgcattccgggcaccagctttagcccggattatcatacccgcccgcattgcgaaagctgccgccattgcaacagcggctttctgattcgcaactgcaccgtgaccgcgaacgcggaatgcagctgcagcaaaaactggcagtgccgcgatcaggaatgcaccgaatgcgatccgccgctgaacccggcgctgacccgccagccgagcgaaaccccgagcccgcagccgccgccgacccatctgccgcatggcaccgaaaaaccgagctggccgctgcatcgccagctgccgaacagcaccgtgtatagccagcgcagcagccatcgcccgctgtgcagcagcgattgcattcgcatttttgtgacctttagcagcatgtttctgatttttgtgctgggcgcgattctgttttttcatcagcgccgcaaccatggcccgaacgaagatcgccaggcggtgccggaagaaccgtgcccgtatagctgcccgcgcgaagaagaaggcagcgcgattccgattcaggaagattatcgcaaaccggaaccggcgttttatccg

SEQ ID NO: 84 - protein, 250 aa, Mus musculus
MAWPPPYWLCMLGTLVGLSATLAPNSCPDKHYWTGGGLCCRMCEPGTFFVKDCEQDRTAAQCDPCIPGTSFSPDYHTRPHCESCRHCNSGFLIRNCTVTANAECSCSKNWQCRDQECTECDPPLNPALTRQPSETPSPQPPPTHLPHGTEKPSWPLHRQLPNSTVYSQRSSHRPLCSSDCIRIFVTFSSMFLIFVLGAILFFHQRRNHGPNEDRQAVPEEPCPYSCPREEEGSAIPIQEDYRKPEPAFYP

SEQ ID NO: 85 - DNA, 660 bp, Homo sapiens
  source 1..660: mol_type = other DNA
atgctgcgcctgctgctggcgctgaacctgtttccgagcattcaggtgaccggcaacaaaattctggtgaaacagagcccgatgctggtggcgtatgataacgcggtgaacctgagctgcaaatatagctataacctgtttagccgcgaatttcgcgcgagcctgcataaaggcctggatagcgcggtggaagtgtgcgtggtgtatggcaactatagccagcagctgcaggtgtatagcaaaaccggctttaactgcgatggcaaactgggcaacgaaagcgtgaccttttatctgcagaacctgtatgtgaaccagaccgatatttatttttgcaaaattgaagtgatgtatccgccgccgtatctggataacgaaaaaagcaacggcaccattattcatgtgaaaggcaaacatctgtgcccgagcccgctgtttccgggcccgagcaaaccgttttgggtgctggtggtggtgggcggcgtgctggcgtgctatagcctgctggtgaccgtggcgtttattattttttgggtgcgcagcaaacgcagccgcctgctgcatagcgattatatgaacatgaccccgcgccgcccgggcccgacccgcaaacattatcagccgtatgcgccgccgcgcgattttgcggcgtatcgcagc

SEQ ID NO: 86 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 87 - DNA, 654 bp, Mus musculus
  source 1..654: mol_type = other DNA
atgaccctgcgcctgctgtttctggcgctgaacttttttagcgtgcaggtgaccgaaaacaaaattctggtgaaacagagcccgctgctggtggtggatagcaacgaagtgagcctgagctgccgctatagctataacctgctggcgaaagaatttcgcgcgagcctgtataaaggcgtgaacagcgatgtggaagtgtgcgtgggcaacggcaactttacctatcagccgcagtttcgcagcaacgcggaatttaactgcgatggcgattttgataacgaaaccgtgacctttcgcctgtggaacctgcatgtgaaccataccgatatttatttttgcaaaattgaatttatgtatccgccgccgtatctggataacgaacgcagcaacggcaccattattcatattaaagaaaaacatctgtgccatacccagagcagcccgaaactgttttgggcgctggtggtggtggcgggcgtgctgttttgctatggcctgctggtgaccgtggcgctgtgcgtgatttggaccaacagccgccgcaaccgcctgctgcagagcgattatatgaacatgaccccgcgccgcccgggcctgacccgcaaaccgtatcagccgtatgcgccggcgcgcgattttgcggcgtatcgcccg

SEQ ID NO: 88 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 89 - DNA, 768 bp, Homo sapiens
  source 1..768: mol_type = other DNA
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtga

SEQ ID NO: 90 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 91 - DNA, 768 bp, Mus musculus
  source 1..768: mol_type = other DNA
atgggcaacaactgctataacgtggtggtgattgtgctgctgctggtgggctgcgaaaaagtgggcgcggtgcagaacagctgcgataactgccagccgggcaccttttgccgcaaatataacccggtgtgcaaaagctgcccgccgagcacctttagcagcattggcggccagccgaactgcaacatttgccgcgtgtgcgcgggctattttcgctttaaaaaattttgcagcagcacccataacgcggaatgcgaatgcattgaaggctttcattgcctgggcccgcagtgcacccgctgcgaaaaagattgccgcccgggccaggaactgaccaaacagggctgcaaaacctgcagcctgggcacctttaacgatcagaacggcaccggcgtgtgccgcccgtggaccaactgcagcctggatggccgcagcgtgctgaaaaccggcaccaccgaaaaagatgtggtgtgcggcccgccggtggtgagctttagcccgagcaccaccattagcgtgaccccggaaggcggcccgggcggccatagcctgcaggtgctgaccctgtttctggcgctgaccagcgcgctgctgctggcgctgatttttattaccctgctgtttagcgtgctgaaatggattcgcaaaaaatttccgcatatttttaaacagccgtttaaaaaaaccaccggcgcggcgcaggaagaagatgcgtgcagctgccgctgcccgcaggaagaagaaggcggcggcggcggctatgaactg

SEQ ID NO: 92 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 93 - DNA, 831 bp, Homo sapiens
  source 1..831: mol_type = other DNA
atgtgcgtgggcgcgcgccgcctgggccgcggcccgtgcgcggcgctgctgctgctgggcctgggcctgagcaccgtgaccggcctgcattgcgtgggcgatacctatccgagcaacgatcgctgctgccatgaatgccgcccgggcaacggcatggtgagccgctgcagccgcagccagaacaccgtgtgccgcccgtgcggcccgggcttttataacgatgtggtgagcagcaaaccgtgcaaaccgtgcacctggtgcaacctgcgcagcggcagcgaacgcaaacagctgtgcaccgcgacccaggataccgtgtgccgctgccgcgcgggcacccagccgctggatagctataaaccgggcgtggattgcgcgccgtgcccgccgggccattttagcccgggcgataaccaggcgtgcaaaccgtggaccaactgcaccctggcgggcaaacataccctgcagccggcgagcaacagcagcgatgcgatttgcgaagatcgcgatccgccggcgacccagccgcaggaaacccagggcccgccggcgcgcccgattaccgtgcagccgaccgaagcgtggccgcgcaccagccagggcccgagcacccgcccggtggaagtgccgggcggccgcgcggtggcggcgattctgggcctgggcctggtgctgggcctgctgggcccgctggcgattctgctggcgctgtatctgctgcgccgcgatcagcgcctgccgccggatgcgcataaaccgccgggcggcggcagctttcgcaccccgattcaggaagaacaggcggatgcgcatagcaccctggcgaaaatt

SEQ ID NO: 94 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 95 - DNA, 816 bp, Mus musculus
  source 1..816: mol_type = other DNA
atgtatgtgtgggtgcagcagccgaccgcgctgctgctgctggcgctgaccctgggcgtgaccgcgcgccgcctgaactgcgtgaaacatacctatccgagcggccataaatgctgccgcgaatgccagccgggccatggcatggtgagccgctgcgatcatacccgcgataccctgtgccatccgtgcgaaaccggcttttataacgaagcggtgaactatgatacctgcaaacagtgcacccagtgcaaccatcgcagcggcagcgaactgaaacagaactgcaccccgacccaggataccgtgtgccgctgccgcccgggcacccagccgcgccaggatagcggctataaactgggcgtggattgcgtgccgtgcccgccgggccattttagcccgggcaacaaccaggcgtgcaaaccgtggaccaactgcaccctgagcggcaaacagacccgccatccggcgagcgatagcctggatgcggtgtgcgaagatcgcagcctgctggcgaccctgctgtgggaaacccagcgcccgacctttcgcccgaccaccgtgcagagcaccaccgtgtggccgcgcaccagcgaactgccgagcccgccgaccctggtgaccccggaaggcccggcgtttgcggtgctgctgggcctgggcctgggcctgctggcgccgctgaccgtgctgctggcgctgtatctgctgcgcaaagcgtggcgcctgccgaacaccccgaaaccgtgctggggcaacagctttcgcaccccgattcaggaagaacataccgatgcgcattttaccctggcgaaaatt

SEQ ID NO: 96 - protein, 272 aa, Mus musculus
MYVWVQQPTALLLLALTLGVTARRLNCVKHTYPSGHKCCRECQPGHGMVSRCDHTRDTLCHPCETGFYNEAVNYDTCKQCTQCNHRSGSELKQNCTPTQDTVCRCRPGTQPRQDSGYKLGVDCVPCPPGHFSPGNNQACKPWTNCTLSGKQTRHPASDSLDAVCEDRSLLATLLWETQRPTFRPTTVQSTTVWPRTSELPSPPTLVTPEGPAFAVLLGLGLGLLAPLTVLLALYLLRKAWRLPNTPKPCWGNSFRTPIQEEHTDAHFTLAKI

SEQ ID NO: 97 - DNA, 597 bp, Homo sapiens
  source 1..597: mol_type = other DNA
atgaaaagcggcctgtggtatttttttctgttttgcctgcgcattaaagtgctgaccggcgaaattaacggcagcgcgaactatgaaatgtttatttttcataacggcggcgtgcagattctgtgcaaatatccggatattgtgcagcagtttaaaatgcagctgctgaaaggcggccagattctgtgcgatctgaccaaaaccaaaggcagcggcaacaccgtgagcattaaaagcctgaaattttgccatagccagctgagcaacaacagcgtgagcttttttctgtataacctggatcatagccatgcgaactattatttttgcaacctgagcatttttgatccgccgccgtttaaagtgaccctgaccggcggctatctgcatatttatgaaagccagctgtgctgccagctgaaattttggctgccgattggctgcgcggcgtttgtggtggtgtgcattctgggctgcattctgatttgctggctgaccaaaaaaaaatatagcagcagcgtgcatgatccgaacggcgaatatatgtttatgcgcgcggtgaacaccgcgaaaaaaagccgcctgaccgatgtgaccctg

SEQ ID NO: 98 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 99 - DNA, 600 bp, Mus musculus
  source 1..600: mol_type = other DNA
atgaaaccgtatttttgccgcgtgtttgtgttttgctttctgattcgcctgctgaccggcgaaattaacggcagcgcggatcatcgcatgtttagctttcataacggcggcgtgcagattagctgcaaatatccggaaaccgtgcagcagctgaaaatgcgcctgtttcgcgaacgcgaagtgctgtgcgaactgaccaaaaccaaaggcagcggcaacgcggtgagcattaaaaacccgatgctgtgcctgtatcatctgagcaacaacagcgtgagcttttttctgaacaacccggatagcagccagggcagctattatttttgcagcctgagcatttttgatccgccgccgtttcaggaacgcaacctgagcggcggctatctgcatatttatgaaagccagctgtgctgccagctgaaactgtggctgccggtgggctgcgcggcgtttgtggtggtgctgctgtttggctgcattctgattatttggtttagcaaaaaaaaatatggcagcagcgtgcatgatccgaacagcgaatatatgtttatggcggcggtgaacaccaacaaaaaaagccgcctggcgggcgtgaccagc

SEQ ID NO: 100 - protein, 200 aa, Mus musculus
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 101 - DNA, 279 bp, Homo sapiens
  source 1..279: mol_type = other DNA
atgattcatctgggccatattctgtttctgctgctgctgccggtggcggcggcgcagaccaccccgggcgaacgcagcagcctgccggcgttttatccgggcaccagcggcagctgcagcggctgcggcagcctgagcctgccgctgctggcgggcctggtggcggcggatgcggtggcgagcctgctgattgtgggcgcggtgtttctgtgcgcgcgcccgcgccgcagcccggcgcaggaagatggcaaagtgtatattaacatgccgggccgcggc

SEQ ID NO: 102 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 103 - DNA, 237 bp, Mus musculus
  source 1..237: mol_type = other DNA
atggatccgccgggctatctgctgtttctgctgctgctgccggtggcggcgagccagaccagcgcgggcagctgcagcggctgcggcaccctgagcctgccgctgctggcgggcctggtggcggcggatgcggtgatgagcctgctgattgtgggcgtggtgtttgtgtgcatgcgcccgcatggccgcccggcgcaggaagatggccgcgtgtatattaacatgccgggccgcggc

SEQ ID NO: 104 - protein, 79 aa, Mus musculus
MDPPGYLLFLLLLPVAASQTSAGSCSGCGTLSLPLLAGLVAADAVMSLLIVGVVFVCMRPHGRPAQEDGRVYINMPGRG

SEQ ID NO: 105 - DNA, 342 bp, Homo sapiens
  source 1..342: mol_type = other DNA
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatga

SEQ ID NO: 106 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 107 - DNA, 345 bp, Mus musculus
  source 1..345: mol_type = other DNA
atgggggctctggagccctcctggtgccttctgttccttcctgtcctcctgactgtgggaggattaagtcccgtacaggcccagagtgacactttcccaagatgcgactgttcttccgtgagccctggtgtactggctgggattgttctgggtgacttggtgttgactctgctgattgccctggctgtgtactctctgggccgcctggtctcccgaggtcaagggacagcggaagggacccggaaacaacacattgctgagactgagtcgccttatcaggagcttcagggtcagagaccagaagtatacagtgacctcaacacacagaggcaatattacagatga

SEQ ID NO: 108 - protein, 114 aa, Mus musculus
MGALEPSWCLLFLPVLLTVGGLSPVQAQSDTFPRCDCSSVSPGVLAGIVLGDLVLTLLIALAVYSLGRLVSRGQGTAEGTRKQHIAETESPYQELQGQRPEVYSDLNTQRQYYR

SEQ ID NO: 109 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 110 - DNA, 492 bp, Homo sapiens
  source 1..492: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 111 - protein, 164 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 112 - DNA, 495 bp, Mus musculus
  source 1..495: mol_type = other DNA
atgaagtggaaagtgtctgttctcgcctgcatcctccacgtgcggttcccaggagcagaggcacagagctttggtctgctggatcccaaactctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 113 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 114 - DNA, 762 bp, Homo sapiens
  source 1..762: mol_type = other DNA
atgtggcagctgctgctgccgaccgcgctgctgctgctggtgagcgcgggcatgcgcaccgaagatctgccgaaagcggtggtgtttctggaaccgcagtggtatcgcgtgctggaaaaagatagcgtgaccctgaaatgccagggcgcgtatagcccggaagataacagcacccagtggtttcataacgaaagcctgattagcagccaggcgagcagctattttattgatgcggcgaccgtggatgatagcggcgaatatcgctgccagaccaacctgagcaccctgagcgatccggtgcagctggaagtgcatattggctggctgctgctgcaggcgccgcgctgggtgtttaaagaagaagatccgattcatctgcgctgccatagctggaaaaacaccgcgctgcataaagtgacctatctgcagaacggcaaaggccgcaaatattttcatcataacagcgatttttatattccgaaagcgaccctgaaagatagcggcagctatttttgccgcggcctgtttggcagcaaaaacgtgagcagcgaaaccgtgaacattaccattacccagggcctggcggtgagcaccattagcagcttttttccgccgggctatcaggtgagcttttgcctggtgatggtgctgctgtttgcggtggataccggcctgtattttagcgtgaaaaccaacattcgcagcagcacccgcgattggaaagatcataaatttaaatggcgcaaagatccgcaggataaa

SEQ ID NO: 115 - protein, 261 aa, Mus musculus
MFQNAHSGSQWLLPPLTILLLFAFADRQSAALPKAVVKLDPPWIQVLKEDMVTLMCEGTHNPGNSSTQWFHNGRSIRSQVQASYTFKATVNDSGEYRCQMEQTRLSDPVDLGVISDWLLLQTPQRVFLEGETITLRCHSWRNKLLNRISFFHNEKSVRYHHYKSNFSIPKANHSHSGDYYCKGSLGSTQHQSKPVTITVQDPATTSSISLVWYHTAFSLVMCLLFAVDTGLYFYVRRNLQTPREYWRKSLSIRKHQAPQDK

SEQ ID NO: 116 - DNA, 786 bp, Mus musculus
  source 1..786: mol_type = other DNA
atgtttcagaatgcacactctggaagccaatggctacttccaccactgacaattctgctgctgtttgcttttgcagacaggcagagtgcagctcttccgaaggctgtggtgaaactggaccccccatggatccaggtgctcaaggaagacatggtgacactgatgtgcgaagggacccacaaccctgggaactcttctacccagtggttccacaacgggaggtccatccggagccaggtccaagccagttacacgtttaaggccacagtcaatgacagtggagaatatcggtgtcaaatggagcagacccgcctcagcgaccctgtagatctgggagtgatttctgactggctgctgctccagacccctcagcgggtgtttctggaaggggaaaccatcacgctaaggtgccatagctggaggaacaaactactgaacaggatctcattcttccataatgaaaaatccgtgaggtatcatcactacaaaagtaatttctctatcccaaaagccaaccacagtcacagtggggactactactgcaaaggaagtctaggaagtacacagcaccagtccaagcctgtcaccatcactgtccaagatccagcaactacatcctccatctctctagtctggtaccacactgctttctccctagtgatgtgcctcctgtttgcagtggacacgggcctttatttctacgtacggagaaatcttcaaaccccgagggagtactggaggaagtccctgtcaatcagaaagcaccaggctcctcaagacaagtga

SEQ ID NO: 117 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 118 - DNA, 648 bp, Homo sapiens
  source 1..648: mol_type = other DNA
atgggctggattcgcggccgccgcagccgccatagctgggaaatgagcgaatttcataactataacctggatctgaaaaaaagcgattttagcacccgctggcagaaacagcgctgcccggtggtgaaaagcaaatgccgcgaaaacgcgagcccgttttttttttgctgctttattgcggtggcgatgggcattcgctttattattatggtggcgatttggagcgcggtgtttctgaacagcctgtttaaccaggaagtgcagattccgctgaccgaaagctattgcggcccgtgcccgaaaaactggatttgctataaaaacaactgctatcagttttttgatgaaagcaaaaactggtatgaaagccaggcgagctgcatgagccagaacgcgagcctgctgaaagtgtatagcaaagaagatcaggatctgctgaaactggtgaaaagctatcattggatgggcctggtgcatattccgaccaacggcagctggcagtgggaagatggcagcattctgagcccgaacctgctgaccattattgaaatgcagaaaggcgattgcgcgctgtatgcgagcagctttaaaggctatattgaaaactgcagcaccccgaacacctatatttgcatgcagcgcaccgtg

SEQ ID NO: 119 - protein, 232 aa, Mus musculus
MALIRDRKSHHSEMSKCHNYDLKPAKWDTSQEQQKQRLALTTSQPGENGIIRGRYPIEKLKISPMFVVRVLAIALAIRFTLNTLMWLAIFKETFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 120 - DNA, 696 bp, Mus musculus
  source 1..696: mol_type = other DNA
atggcgctgattcgcgatcgcaaaagccatcatagcgaaatgagcaaatgccataactatgatctgaaaccggcgaaatgggataccagccaggaacagcagaaacagcgcctggcgctgaccaccagccagccgggcgaaaacggcattattcgcggccgctatccgattgaaaaactgaaaattagcccgatgtttgtggtgcgcgtgctggcgattgcgctggcgattcgctttaccctgaacaccctgatgtggctggcgatttttaaagaaacctttcagccggtgctgtgcaacaaagaagtgccggtgagcagccgcgaaggctattgcggcccgtgcccgaacaactggatttgccatcgcaacaactgctatcagttttttaacgaagaaaaaacctggaaccagagccaggcgagctgcctgagccagaacagcagcctgctgaaaatttatagcaaagaagaacaggattttctgaaactggtgaaaagctatcattggatgggcctggtgcagattccggcgaacggcagctggcagtgggaagatggcagcagcctgagctataaccagctgaccctggtggaaattccgaaaggcagctgcgcggtgtatggcagcagctttaaagcgtataccgaagattgcgcgaacctgaacacctatatttgcatgaaacgcgcggtg

SEQ ID NO: 121 - protein, 4 aa, synthetic construct
YMNM

SEQ ID NO: 122 - protein, 4 aa, synthetic construct
PYAP

SEQ ID NO: 123 - protein, 4 aa, synthetic construct
FMNM

SEQ ID NO: 124 - protein, 4 aa, synthetic construct
AYAA

SEQ ID NO: 125 - protein, 19 aa, synthetic construct
MGWSCIILFLVATATGVHS

SEQ ID NO: 126 - DNA, 57 bp, synthetic construct
  source 1..57: mol_type = other DNA
atgggatggagctgtatcatcctcttcttggtagcaacagctaccggtgtgcactcc

SEQ ID NO: 127 - protein, 276 aa, Homo sapiens
MRNQAPGRPKGATFPPRRPTGSRAPPLAPELRAKQRPGERVMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 128 - DNA, 828 bp, Homo sapiens
  source 1..828: mol_type = other DNA
atgcgcaaccaggcgccgggccgcccgaaaggcgcgacctttccgccgcgccgcccgaccggcagccgcgcgccgccgctggcgccggaactgcgcgcgaaacagcgcccgggcgaacgcgtgatggcgctgccggtgaccgcgctgctgctgccgctggcgctgctgctgcatgcggcgcgcccgagccagtttcgcgtgagcccgctggatcgcacctggaacctgggcgaaaccgtggaactgaaatgccaggtgctgctgagcaacccgaccagcggctgcagctggctgtttcagccgcgcggcgcggcggcgagcccgacctttctgctgtatctgagccagaacaaaccgaaagcggcggaaggcctggatacccagcgctttagcggcaaacgcctgggcgatacctttgtgctgaccctgagcgattttcgccgcgaaaacgaaggctattatttttgcagcgcgctgagcaacagcattatgtattttagccattttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccgccgccgcgtgtgcaaatgcccgcgcccggtggtgaaaagcggcgataaaccgagcctgagcgcgcgctatgtg

SEQ ID NO: 129 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 130 - DNA, 741 bp, Mus musculus
  source 1..741: mol_type = other DNA
atggcgagcccgctgacccgctttctgagcctgaacctgctgctgatgggcgaaagcattattctgggcagcggcgaagcgaaaccgcaggcgccggaactgcgcatttttccgaaaaaaatggatgcggaactgggccagaaagtggatctggtgtgcgaagtgctgggcagcgtgagccagggctgcagctggctgtttcagaacagcagcagcaaactgccgcagccgacctttgtggtgtatatggcgagcagccataacaaaattacctgggatgaaaaactgaacagcagcaaactgtttagcgcggtgcgcgataccaacaacaaatatgtgctgaccctgaacaaatttagcaaagaaaacgaaggctattatttttgcagcgtgattagcaacagcgtgatgtattttagcagcgtggtgccggtgctgcagaaagtgaacagcaccaccaccaaaccggtgctgcgcaccccgagcccggtgcatccgaccggcaccagccagccgcagcgcccggaagattgccgcccgcgcggcagcgtgaaaggcaccggcctggattttgcgtgcgatatttatatttgggcgccgctggcgggcatttgcgtggcgccgctgctgagcctgattattaccctgatttgctatcatcgcagccgcaaacgcgtgtgcaaatgcccgcgcccgctggtgcgccaggaaggcaaaccgcgcccgagcgaaaaaattgtg

SEQ ID NO: 131 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 132 - DNA, 831 bp, Homo sapiens
  source 1..831: mol_type = other DNA
atggtgcggctgcccctgcagtgtgtgctgtggggctgcctcctgaccgccgtgcaccccgaaccacccacagcatgccgtgaaaagcagtatctgatcaactctcagtgctgtagcctgtgtcaacctggacagaaacttgtgtccgattgtacggagttcaccgaaaccgaatgtctcccttgcggagagtccgagttcctggacacctggaatcgcgaaacccactgccatcagcacaagtattgtgatccaaacctgggcctcagggtgcaacagaagggaactagtgagacggacaccatctgtacctgtgaagaggggtggcactgcacctctgaggcgtgtgagtcatgcgtgttgcacagatcctgttctccgggcttcggtgtcaagcaaatcgccaccggcgtgtccgacactatctgtgagccatgtccagtgggcttctttagcaatgtgtctagcgccttcgagaagtgtcacccgtggacctcctgtgaaactaaggacctggtcgttcaacaggccggcaccaataaaactgatgtggtctgtggccctcaggaccgcctccgtgccctcgtggtcatcccgatcattttcggtatcctgttcgccattttgctggtactcgtgttcatcaaaaaggtggcgaaaaagccaaccaacaaagcgccacaccccaaacaagaaccacaagagatcaacttcccagatgacctccctggatccaacaccgcagcccctgtgcaggagaccctccacgggtgtcaaccagtgactcaggaagatggtaaggagtcccgcatttccgtgcaggaaaggcag

SEQ ID NO: 133 - protein, 289 aa, Mus musculus
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 134 - DNA, 867 bp, Mus musculus
  source 1..867: mol_type = other DNA
atggtgtccttgccccggctgtgcgcattgtggggttgcctgttgactgccgtgcacttgggtcagtgcgtgacctgctccgataagcagtacctccacgacggccaatgctgtgacctgtgtcagcccggttccaggctgaccagccactgcaccgccctggagaagacccagtgccatccctgcgactccggcgagttctccgctcagtggaaccgcgagatccgctgccatcagcaccgtcactgcgagcctaaccagggcctgagagtcaagaaagagggcaccgctgagtccgataccgtctgtacctgcaaggagggccagcattgcacctcaaaggactgtgaggcttgtgcccagcacacaccctgcatccctggttttggagttatggagatggccaccgaaacaaccgatactgtatgtcatccctgccctgtgggatttttcagcaaccagtcctctctgttcgaaaagtgctacccctggaccagctgtgaagataagaacctggaggttctgcagaagggtacctcccaaacaaacgtgatctgcggcctgaagagcaggatgcgtgctcttctggtgatcccggtcgtaatgggcatcctgatcaccatctttggggtgttcctgtatatcaaaaaggtagtgaaaaagcctaaggacaacgaaatcctgccacctgctgcccgtaggcaggatccccaggagatggaggactaccctggtcataacactgctgcaccagtgcaggagacacttcatggatgccagcccgtgacacaggaagacggaaaggaaagccgcatctccgtccaggaacgccaggtcacagattccatcgccctgcgcccgctcgtg

SEQ ID NO: 135 - protein, 19 aa, synthetic construct
MGWSCIILFLVATATGVHS

SEQ ID NO: 136 - protein, 48 aa, Homo sapiens
AKPTTTPAPRPPTPAPTIASQPLSLRPEACRPAAGGAVHTRGLDFACD

SEQ ID NO: 137 - protein, 21 aa, Homo sapiens
IYIWAPLAGTCGVLLLSLVIT

SEQ ID NO: 138 - protein, 42 aa, Homo sapiens; identical to SEQ ID NO: 76

SEQ ID NO: 139 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 140 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 141 - protein, 21 aa, synthetic construct
GSGEGRGSLLTCGDVEENPGP

SEQ ID NO: 142 - protein, 23 aa, synthetic construct
GSGQCTNYALLKLAGDVESNPGP

SEQ ID NO: 143 - protein, 750 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 144 - protein, 731 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 145 - protein, 491 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 146 - protein, 472 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 147 - protein, 752 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGQCTNYALLKLAGDVESNPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 148 - protein, 496 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 149 - protein, 477 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 150 - protein, 755 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 151 - protein, 431 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - protein, 1359 aa, synthetic construct
ATGGCCCTGCCTGTGACAGCCCTACTACTGCCCCTGGCCCTTCTGCTTCATGCTGCTAGACCTGAGGTGCAGCTGGTGGAATCTGGGGGGGGCTTGGTTCAGCCTGGGGGCAGCCTGAGACTGAGCTGTGCTGCCTCTGGCTTCACCTTCAGCAGATATTGGATGAACTGGGTGAGACAAGCCCCTGGCAAGGGCCTGGAGTGGGTGGGGGAGATCACCCCTGACAGCAGCACCATCAACTATGCCCCTAGCCTGAAGGGCAGATTCACCATCAGCAGAGACAATGCCAAGAACAGCCTGTACCTGCAGATGAACAGCCTGAGAGCTGAGGACACAGCTGTGTACTATTGTGCTAGACCCTATGACTATGGGGCCTGGTTTGCTAGCTGGGGCCAAGGCACCCTAGTAACAGTGTCATCTGGGGGGGGAGGATCTGGGGGGGGGGGTTCTGGGGGGGGGGGCTCTGGTGGGGGGGGTTCTCAAGCTGTGGTAACACAAGAGCCTAGCCTGACAGTGAGCCCTGGGGGCACAGTGACCCTGACCTGCAGAAGCAGCACTGGGGCTGTGACCACAAGCAACTATGCCAACTGGGTGCAAGAGAAGCCTGACCACCTGTTCACTGGCCTGATTGGGGGCACCAATAAGAGAGCACCTGGCACTCCTGCTAGATTTTCTGGCTCACTGCTGGGGGGCAAGGCTGCCTTGACCCTTTCTGGGGCTCAGCCTGAGGATGAGGCTGAGTACTACTGTGCTCTCTGGTACAGCAACCACTGGGTGTTTGGGGGGGGCACCAAGCTGACAGTGCTGGGGGGGGGGGGTAGCATCTACATCTGGGCCCCCCTGGCTGGCACATGTGGGGTGCTGCTGCTGAGCCTGGTGATCACCCTGTACTGCAAGAGAGGCAGAAAGAAGCTGCTGTACATCTTCAAGCAGCCCTTCATGAGACCTGTGCAGACCACCCAAGAGGAGGATGGCTGCAGCTGCAGATTCCCTGAGGAGGAGGAGGGGGGCTGTGAGCTGAGAGTGAAGTTCAGCAGATCTGCTGATGCCCCTGCCTATCAGCAAGGGCAGAATCAGCTGTATAATGAGCTCAACCTGGGCAGAAGAGAGGAGTATGATGTGCTGGACAAGAGAAGAGGCAGAGACCCTGAGATGGGGGGCAAGCCTAGAAGAAAGAACCCCCAAGAGGGCCTGTACAATGAGCTGCAAAAGGACAAGATGGCTGAGGCCTACTCTGAGATTGGCATGAAGGGGGAGAGAAGAAGAGGCAAGGGCCATGATGGCCTGTACCAAGGCCTGAGCACAGCCACCAAGGACACTTATGATGCCTTGCACATGCAAGCCCTGCCTCCAAGATGA

SEQ ID NO: 153 - protein, 24 aa, Homo sapiens
IYIWAPLAGTCGVLLLSLVITLYC

SEQ ID NO: 154 - protein, 440 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 1386 aa, synthetic construct
ATGGCCCTGCCTGTGACAGCCCTATTACTGCCCCTGGCCCTTCTGTTACATGCTGCTAGACCTGAGGTTCAACTGGTGGAGTCTGGGGGGGGCCTAGTGCAGCCTGGGGGCAGCCTGAGACTGAGCTGTGCTGCCTCTGGCTTCACCTTCAGCAGATACTGGATGAACTGGGTGAGACAAGCCCCTGGCAAGGGCCTGGAGTGGGTGGGGGAGATCACCCCTGACAGCAGCACCATCAACTATGCCCCTAGCCTGAAGGGCAGATTCACCATCAGCAGAGACAATGCCAAGAACAGCCTGTACCTGCAGATGAACAGCCTGAGAGCTGAGGACACAGCTGTGTATTATTGTGCTAGACCATATGACTATGGGGCCTGGTTTGCCTCTTGGGGCCAAGGCACACTGGTTACAGTGAGTTCTGGGGGGGGGGGTTCTGGAGGGGGGGGATCTGGGGGTGGAGGTTCTGGGGGGGGGGGCAGTCAAGCTGTGGTGACCCAAGAGCCTAGCCTGACAGTGTCCCCTGGGGGCACAGTCACCCTGACCTGCAGAAGCAGCACTGGGGCTGTGACCACAAGCAACTATGCCAACTGGGTGCAAGAGAAGCCTGACCACCTGTTCACTGGCCTGATTGGGGGCACCAACAAAAGAGCCCCTGGCACCCCTGCTAGATTCTCTGGAAGCCTGTTGGGGGGCAAGGCTGCCCTGACCCTATCTGGGGCACAGCCTGAGGATGAGGCTGAGTACTACTGTGCCCTCTGGTACAGCAACCACTGGGTGTTTGGGGGGGGCACCAAACTGACAGTGTTGGAGAGCAAGTATGGCCCCCCCTGTCCTCCCTGTCCCTTTTGGGTGCTGGTGGTTGTGGGGGGGGTGCTGGCCTGCTACAGCCTGCTGGTGACAGTGGCCTTCATCATCTTCTGGGTGAGAAGCAAGAGAAGCAGACTGCTGCACTCTGACTACATGAACATGACCCCTAGAAGACCTGGCCCCACAAGAAAGCACTATCAGCCCTATGCCCCCCCTAGAGACTTTGCTGCCTACAGAAGCAGAGTGAAGTTCAGCAGATCTGCTGATGCCCCTGCCTATCAGCAAGGGCAGAATCAGCTGTATAATGAGCTCAACCTGGGCAGAAGAGAGGAGTATGATGTGCTGGACAAGAGAAGAGGCAGAGACCCTGAGATGGGGGGCAAGCCTAGAAGAAAGAACCCCCAAGAGGGCCTGTACAATGAGCTGCAAAAGGACAAGATGGCTGAGGCCTACTCTGAGATTGGCATGAAGGGGGAGAGAAGAAGAGGCAAGGGCCATGATGGCCTGTACCAAGGCCTGAGCACAGCCACCAAGGACACCTATGATGCCCTACATATGCAAGCTCTGCCCCCTAGATGA

SEQ ID NO: 156 - protein, 12 aa, synthetic construct
ESKYGPPCPPCP

SEQ ID NO: 157 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 158 - protein, 115 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLS

SEQ ID NO: 159 - protein, 23 aa, Homo sapiens
VIGFRILLLKVAGFNLLMTLRLW

SEQ ID NO: 160 - skipped

SEQ ID NO: 161 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 162 - protein, 149 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYE

SEQ ID NO: 163 - protein, 21 aa, Homo sapiens
ILLGKATLYAVLVSALVLMAM

SEQ ID NO: 164 - protein, 6 aa, Homo sapiens
VKRKDF

SEQ ID NO: 165 - protein, 178 aa, Homo sapiens
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 166 - protein, 144 aa, Homo sapiens
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSA

SEQ ID NO: 167 - protein, 23 aa, Homo sapiens
TILYEILLGKATLYAVLVSALVL

SEQ ID NO: 168 - protein, 11 aa, Homo sapiens
MAMVKRKDSRG

SEQ ID NO: 169 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 170 - protein, 138 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSA

SEQ ID NO: 171 - protein, 23 aa, Homo sapiens
YYMYLLLLLKSVVYFAIITCCLL

SEQ ID NO: 172 - protein, 12 aa, Homo sapiens
RRTAFCCNGEKS

SEQ ID NO: 173 - protein, 189 aa, Homo sapiens
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 174 - protein, 154 aa, Homo sapiens
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSA

SEQ ID NO: 175 - protein, 23 aa, Homo sapiens
YYTYLLLLLKSVVYFAIITCCLL

SEQ ID NO: 176 - protein, 12 aa, Homo sapiens
RRTAFCCNGEKS

SEQ ID NO: 177 - protein, 153 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 178 - protein, 129 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 179 - protein, 23 aa, Homo sapiens
LGLRMLFAKTVAVNFLLTAKLFF

SEQ ID NO: 180 - skipped

SEQ ID NO: 181 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 182 - protein, 22 aa, Homo sapiens
MQSGTHWRVLGLCLLSVGVWGQ

SEQ ID NO: 183 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 184 - protein, 26 aa, Homo sapiens
VMSVATIVIVDICITGGLLLLVYYWS

SEQ ID NO: 185 - protein, 55 aa, Homo sapiens
KNRKAKAKPVTRGAGAGGRQRGQNKERPPPVPNPDYEPIRKGQRDLYSGLNQRRI

SEQ ID NO: 186 - protein, 185 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 187 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 188 - protein, 21 aa, Homo sapiens
MEHSTFLSGLVLATLLSQVSP

SEQ ID NO: 189 - protein, 84 aa, Homo sapiens [sequence omitted]

SEQ ID NO: 190 - protein, 21 aa, Homo sapiens
GIIVTDVIATLLLALGVFCFA

SEQ ID NO: 191 - protein, 45 aa, Homo sapiens
GHETGRLSGAADTQALLRNDQVYQPLRDRDDAQYSHLGGNWARNK

SEQ ID NO: 192 - protein, 150 aa, Homo sapiens
FKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 193 - protein, 171 aa, Homo sapiens; identical to SEQ ID NO: 187

SEQ ID NO: 194 - protein, 21 aa, Homo sapiens
MEHSTFLSGLVLATLLSQVSP

SEQ ID NO: 195 - protein, 84 aa, Homo sapiens [sequence omitted]

SEQ ID NO: 196 - protein, 21 aa, Homo sapiens
GIIVTDVIATLLLALGVFCFA

SEQ ID NO: 197 - protein, 45 aa, Homo sapiens; identical to SEQ ID NO: 191

SEQ ID NO: 198 - protein, 150 aa, Homo sapiens; identical to SEQ ID NO: 192

SEQ ID NO: 199 - protein, 164 aa, Homo sapiens; identical to SEQ ID NO: 109

SEQ ID NO: 200 - protein, 21 aa, Homo sapiens
MKWKALFTAAILQAQLPITEA

SEQ ID NO: 201 - protein, 9 aa, Homo sapiens
QSFGLLDPK

SEQ ID NO: 202 - protein, 21 aa, Homo sapiens
LCYLLDGILFIYGVILTALFL

SEQ ID NO: 203 - protein, 113 aa, Homo sapiens; identical to SEQ ID NO: 139

SEQ ID NO: 204 - protein, 143 aa, Homo sapiens
QSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 205 - protein, 163 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 206 - protein, 112 aa, Homo sapiens; identical to SEQ ID NO: 75

SEQ ID NO: 207 - protein, 142 aa, Homo sapiens
QSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 208 - protein, 140 aa, Homo sapiens
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 209 - protein, 176 aa, Homo sapiens
DLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 210 - protein, 178 aa, Homo sapiens
DLKNVFPPKVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 211 - protein, 721 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 212 - protein, 702 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 213 - protein, 462 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 214 - protein, 443 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 215 - protein, 721 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 216 - protein, 702 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 217 - protein, 462 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 218 - protein, 443 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 219 - protein, 721 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 220 - protein, 702 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 221 - protein, 462 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 222 - protein, 443 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 223 - protein, 723 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGQCTNYALLKLAGDVESNPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 224 - protein, 537 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 225 - protein, 518 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 226 - protein, 278 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 227 - protein, 259 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 228 - protein, 537 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 229 - protein, 518 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 230 - protein, 278 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 231 - protein, 259 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 232 - protein, 537 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 233 - protein, 518 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 234 - protein, 278 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 235 - protein, 259 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 236 - protein, 527 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 237 - protein, 508 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 238 - protein, 268 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 239 - protein, 249 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 240 - protein, 573 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 241 - protein, 554 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 242 - protein, 314 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 243 - protein, 295 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 244 - protein, 573 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 245 - protein, 554 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 246 - protein, 314 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 247 - protein, 295 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 248 - protein, 573 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 249 - protein, 554 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 250 - protein, 314 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 251 - protein, 295 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 252 - protein, 563 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 253 - protein, 544 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 254 - protein, 304 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 255 - protein, 285 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 256 - protein, 862 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 257 - protein, 603 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 258 - protein, 862 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 259 - protein, 603 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 260 - protein, 862 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 261 - protein, 603 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 262 - protein, 862 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 263 - protein, 603 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 264 - protein, 862 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 265 - protein, 603 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 266 - protein, 862 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 267 - protein, 603 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 268 - protein, 894 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 269 - protein, 635 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 270 - protein, 894 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 271 - protein, 635 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 272 - protein, 894 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 273 - protein, 635 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 274 - protein, 894 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 275 - protein, 635 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 276 - protein, 894 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 277 - protein, 635 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 278 - protein, 894 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 279 - protein, 635 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 280 - RNA, 100 nt, synthetic construct
  source 1..100: mol_type = other RNA
ggtggccacaattgtcataggttttagagctagaaatagcaagttaaaataaggctagtccgttatcaacttgaaaaagtggcaccgagtcggtgctttt

SEQ ID NO: 281 - RNA, 100 nt, synthetic construct
  source 1..100: mol_type = other RNA
ggagaatgacgagtggacccgttttagagctagaaatagcaagttaaaataaggctagtccgttatcaacttgaaaaagtggcaccgagtcggtgctttt

SEQ ID NO: 282 - RNA, 100 nt, synthetic construct
  source 1..100: mol_type = other RNA
agagtctctcagctggtacagttttagagctagaaatagcaagttaaaataaggctagtccgttatcaacttgaaaaagtggcaccgagtcggtgctttt

SEQ ID NO: 283 - protein, 22 aa, synthetic construct
GSGATNFSLLKQAGDVEENPGP

SEQ ID NO: 284 - protein, 25 aa, synthetic construct
GSGVKQTLNFDLLKLAGDVESNPGP

SEQ ID NO: 285 - protein, 25 aa, synthetic construct
YXXLIWHEREINXANYAMINOACID

SEQ ID NO: 286 - protein, 31 aa, synthetic construct
YXXLIXYXXLIWHEREINXANYAMINOACID

SEQ ID NO: 287 - DNA, 1329 bp, synthetic construct
  source 1..1329: mol_type = other DNA
gaagtgcagctggttgagtctggcggaggactggttcaacctggcggaagcctgagactgtcttgtgccgccagcggcttcaccttcagcagatactggatgaactgggtgagacaggcccctggcaaaggactggagtgggtgggagagatcacccctgacagcagcaccatcaattacgcccctagcctgaagggcagattcaccatctccagagacaacgccaagaacagcctgtacctgcagatgaactccctgagagccgaggacaccgccgtgtactactgtgccagaccttacgattacggcgcctggtttgcttcttggggccagggaacactggtgacagttagctctggaggaggaggatcaggcggaggcggaagtggtggtggaggatctcaggctgtggtgacacaagagccaagcctgacagtgtctccaggcggcacagtgaccctgacctgtagatcttctaccggagccgtgaccaccagcaactacgccaattgggtgcaagagaagcccgaccacctgtttacaggcctgatcggcggcaccaataagagagcacctggcacaccagccagattctctggatctctgctcggaggaaaggccgctctgacactgtctggagcccagccagaggatgaggccgagtattattgtgccctgtggtacagcaaccactgggtgtttggcggcggaacaaagctgacagttcttggaggtggtggaagcggtggaggtggatctggaggtggcggatccgatggaaatgaggagatgggcggcatcacccagacaccttacaaggtgagcatcagcggcaccaccgtgatcctgacatgtcctcagtaccctggcagcgagatcctgtggcagcacaacgataagaacatcggcggcgacgaggacgacaagaatatcggctctgacgaggatcacctgtctctgaaggagttcagcgagctggagcagagcggctactacgtgtgttaccctagaggcagcaagcccgaggacgccaacttctacctgtatctgagagccagagtgtgcgagaactgcatggagatggatgtgatgagtgtcgctaccatcgtgatcgtcgacatctgtatcacaggcggcctgctgctgctggtgtactattggagcaagaaccggaaggccaaagccaagccagtgacaagaggagcaggagccggaggtagacagagaggccagaacaaagaaaggcctccaccggtgcctaatcctgactacgagcccatcagaaagggccagagagatctgtacagcggcctgaaccagagaagaatc

SEQ ID NO: 288 - DNA, 777 bp, synthetic construct
  source 1..777: mol_type = other DNA
gaggtgcagctggtggagagcggcggcggcctggtgcagcccggcggcagcctgaggctgagctgcgccgccagcggcttcaccttcagcaggtactggatgaactgggtgaggcaggcccccggcaagggcctggagtgggtgggcgagatcacccccgacagcagcaccatcaactacgcccccagcctgaagggcaggttcaccatcagcagggacaacgccaagaacagcctgtacctgcagatgaacagcctgagggccgaggacaccgccgtgtactactgcgccaggccctacgactacggcgcctggttcgccagctggggccagggcaccctggtgaccgtgagcagcatccagaaccccgaccccgcagtctatcaattgcgtgatagtaagagtagcgacaagtctgtgtgcctgttcaccgatttcgacagtcagacaaatgtttctcagagtaaggacagtgatgtgtacatcaccgacaaatgcgtcctggacatgcgctccatggacttcaagagtaatagcgccgtagcctggagcaacaaaagcgacttcgcatgtgccaacgcgttcaataactccattatccctgaagatacttttttcccctcccccgaaagtagctgcgatgttaaactcgttgagaagtccttcgaaactgacaccaacctgaacttccagaacctgagtgtgattggcttccgcatcctgctcctgaaagtggccggctttaacctgctcatgacactgcgtctgtggtcaagc

SEQ ID NO: 289 - DNA, 855 bp, synthetic construct
  source 1..855: mol_type = other DNA
caggccgtggtgacccaggagcccagcctgaccgtgagccccggcggcaccgtgaccctgacctgcaggagcagcaccggcgccgtgaccaccagcaactacgccaactgggtgcaggagaagcccgaccacctgttcaccggcctgatcggcggcaccaacaagagggcccccggcacccccgccaggttcagcggcagcctgctgggcggcaaggccgccctgaccctgagcggcgcccagcccgaggacgaggccgagtactactgcgccctgtggtacagcaaccactgggtgttcggcggcggcaccaagctgaccgtcctagacctgaataaagtgtttcccccagaggtcgccgtgtttgaaccgagcgaggctgagatctcccacacccagaaggctacactggtctgtcttgccaccggtttctttcctgaccacgtggaactgtcttggtgggttaacggtaaggaggtgcactccggtgtgtgcactgatccccagccgttgaaagaacagccggctttgaacgactcccggtactgccttagctcccgcctgcgcgtgtctgctacattctggcagaacccccgcaaccactttcggtgccaggtgcagttttacggcctgtccgagaacgatgaatggacacaagaccgggctaagcccgtgactcagatcgtgagtgctgaggcttgggggcgcgccgattgcggcttcacctcagtcagctaccaacagggcgttctgagtgccacaatcctgtatgagatcctcctgggaaaggcaactctgtacgctgttctcgtctccgcgcttgttctgatggctatggtgaagagaaaggatttc

SEQ ID NO: 290 - DNA, 747 bp, synthetic construct
  source 1..747: mol_type = other DNA
caggccgtggtgacccaggagcccagcctgaccgtgagccccggcggcaccgtgaccctgacctgcaggagcagcaccggcgccgtgaccaccagcaactacgccaactgggtgcaggagaagcccgaccacctgttcaccggcctgatcggcggcaccaacaagagggcccccggcacccccgccaggttcagcggcagcctgctgggcggcaaggccgccctgaccctgagcggcgcccagcccgaggacgaggccgagtactactgcgccctgtggtacagcaaccactgggtgttcggcggcggcaccaagctgaccgtcctaatccagaaccccgaccccgcagtctatcaattgcgtgatagtaagagtagcgacaagtctgtgtgcctgttcaccgatttcgacagtcagacaaatgtttctcagagtaaggacagtgatgtgtacatcaccgacaaatgcgtcctggacatgcgctccatggacttcaagagtaatagcgccgtagcctggagcaacaaaagcgacttcgcatgtgccaacgcgttcaataactccattatccctgaagatacttttttcccctcccccgaaagtagctgcgatgttaaactcgttgagaagtccttcgaaactgacaccaacctgaacttccagaacctgagtgtgattggcttccgcatcctgctcctgaaagtggccggctttaacctgctcatgacactgcgtctgtggtcaagc

SEQ ID NO: 291 - DNA, 885 bp, synthetic construct
  source 1..885: mol_type = other DNA
gaggtgcagctggtggagagcggcggcggcctggtgcagcccggcggcagcctgaggctgagctgcgccgccagcggcttcaccttcagcaggtactggatgaactgggtgaggcaggcccccggcaagggcctggagtgggtgggcgagatcacccccgacagcagcaccatcaactacgcccccagcctgaagggcaggttcaccatcagcagggacaacgccaagaacagcctgtacctgcagatgaacagcctgagggccgaggacaccgccgtgtactactgcgccaggccctacgactacggcgcctggttcgccagctggggccagggcaccctggtgaccgtgagcagcgacctgaataaagtgtttcccccagaggtcgccgtgtttgaaccgagcgaggctgagatctcccacacccagaaggctacactggtctgtcttgccaccggtttctttcctgaccacgtggaactgtcttggtgggttaacggtaaggaggtgcactccggtgtgtgcactgatccccagccgttgaaagaacagccggctttgaacgactcccggtactgccttagctcccgcctgcgcgtgtctgctacattctggcagaacccccgcaaccactttcggtgccaggtgcagttttacggcctgtccgagaacgatgaatggacacaagaccgggctaagcccgtgactcagatcgtgagtgctgaggcttgggggcgcgccgattgcggcttcacctcagtcagctaccaacagggcgttctgagtgccacaatcctgtatgagatcctcctgggaaaggcaactctgtacgctgttctcgtctccgcgcttgttctgatggctatggtgaagagaaaggatttc

SEQ ID NO: 292 - protein, 5 aa, synthetic construct
NAWMS

SEQ ID NO: 293 - protein, 19 aa, synthetic construct
RIKSKTDGGTTDYAAPVKG

SEQ ID NO: 294 - protein, 9 aa, synthetic construct
PWEWSWYDY

SEQ ID NO: 295 - protein, 14 aa, synthetic construct
GSSTGAVTTSNYAN

SEQ ID NO: 296 - protein, 7 aa, synthetic construct
GTNKRAP

SEQ ID NO: 297 - protein, 9 aa, synthetic construct
ALWYSNLWV

SEQ ID NO: 298 - protein, 120 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSS

SEQ ID NO: 299 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 300 - protein, 450 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 301 - protein, 215 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 302 - protein, 5 aa, synthetic construct
DTYMH

SEQ ID NO: 303 - protein, 17 aa, synthetic construct
RIDPANGNSKYVPKFQG

SEQ ID NO: 304 - protein, 12 aa, synthetic construct
FGYYVSDYAMAY

SEQ ID NO: 305 - protein, 15 aa, synthetic construct
RAGESVDIFGVGFLH

SEQ ID NO: 306 - protein, 7 aa, synthetic construct
RASNLES

SEQ ID NO: 307 - protein, 9 aa, synthetic construct
QQTNEDPYT

SEQ ID NO: 308 - protein, 121 aa, synthetic construct
EVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSS

SEQ ID NO: 309 - protein, 111 aa, synthetic construct
DIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIK

SEQ ID NO: 310 - protein, 450 aa, synthetic construct
EVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 311 - protein, 218 aa, synthetic construct
DIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 312 - protein, 5 aa, synthetic construct
SYAMS

SEQ ID NO: 313 - protein, 17 aa, synthetic construct
AISGSGGSTYYADSVKG

SEQ ID NO: 314 - protein, 6 aa, synthetic construct
GSGFDY

SEQ ID NO: 315 - protein, 12 aa, synthetic construct
RASQSVSSSYLA

SEQ ID NO: 316 - protein, 7 aa, synthetic construct
GASSRAT

SEQ ID NO: 317 - protein, 9 aa, synthetic construct
QQYGSSPLT

SEQ ID NO: 318 - protein, 115 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSS

SEQ ID NO: 319 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 320 - protein, 445 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 322 - protein, 599 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGGGSGGGGSGGQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 323 - protein, 213 aa, synthetic construct
DIQMTQSPSSLSASVGDRVTITCRASQSIGRYLAWYQQKPGKAPKLLIYSASNLETGVPSRFSGSGSGTDFTLTISSLQPEDFATYYCQQYNSFPVTFGPGTKVDIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 324 - protein, 449 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 325 - protein, 218 aa, synthetic construct
DIVMTQSPDSLAVSLGERATINCKASESVDTSDNSFIHWYQQKPGQSPKLLIYRASTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 326 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 327 - protein, 214 aa, synthetic construct
DIQMTQSPSSLSASVGDRVTITCRASQSIGRYLAWYQQKPGKAPKLLIYSASNLETGVPSRFSGSGSGTDFTLTISSLQPEDFATYYCQQYNSFPVTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 328 - protein, 595 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGGGSGGGGSGGQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0091062 A1
(43) Pub. Date: Apr. 2, 2026

(54) Recombinant Fc domain - IL2 variant polypeptides and combination therapy with membrane-anchored antigen binding polypeptides

(71) Applicant: Hoffmann-La Roche Inc., Little Falls, NJ (US)

(72) Inventors: Diana DAROWSKI, Singapore (SG); Stephan GASSER, Fislisbach (CH); Christian KLEIN, Bonstetten (CH); Simone LANG, Zuerich (CH); Dario VENETZ, Bonstetten (CH)

(73) Assignee: Hoffmann-La Roche Inc., Little Falls, NJ (US)

(21) Appl. No.: 19/272,092

(22) Filed: Jul. 17, 2025

Related U.S. Application Data
(63) Continuation of application No. PCT/EP2024/051096, filed on Jan. 18, 2024.

(30) Foreign Application Priority Data
Jan. 20, 2023 (EP) 23152578.3

(51) Int. Cl. A61K 35/17; A61K 40/11; A61K 40/31; A61K 40/42; A61P 35/00; A61P 37/04; C07K 14/55; C07K 14/705; C07K 14/715; C07K 14/725; C07K 14/735; C12N 5/00

(52) U.S. Cl. CPC A61K 35/17; A61K 40/11; A61K 40/31; A61K 40/4217; A61K 40/4234; A61P 35/00; A61P 37/04; C07K 14/55; C07K 14/7051; C07K 14/70517; C07K 14/70535; C07K 14/7155; C12N 5/0018; A61K 2239/11; C07K 2319/30; C12N 2501/2302; C12N 2501/515

(57) ABSTRACT

The present invention relates to the combination therapy of recombinant Fc domain-IL-2 variant polypeptides with membrane-anchored antigen binding polypeptides in the prevention or treatment of cancer.

### FIELD OF THE INVENTION

The present invention relates to the combination therapy of recombinant Fc domain-IL-2 variant polypeptides with membrane-anchored antigen binding polypeptides in the prevention or treatment of cancer.

### BACKGROUND OF THE INVENTION

Adoptive cell therapy has become a clinically validated approach in cancer treatment. Although chimeric antigen receptor (CAR)-T cell therapy has shown clinical efficacy in hematological malignancies, there are several hurdles that still have to be addressed in order to be efficacious in solid tumors. A key challenge for solid tumor CAR-T therapy is on-target off-tumor toxicity, which is triggered by limited but significant expression of the tumor antigen in healthy tissues. As a consequence, CAR-T cell-induced systemic cytokine release is often observed and can lead to a variety of therapy-related symptoms, including neurotoxicity.

Adaptor-based CARs comprise a tumor antigen-specific adapter molecule and a CAR with exclusive specificity for the adapter molecule. The corresponding adapter CAR-T cells can only be activated in the presence of an adapter molecule bound to antigen-positive cells, which allows controlling their therapeutic activity and systemic toxicity. We have previously reported a modular adapter CAR-T approach, using recombinant antibodies featuring mutated effector function-silent Fc domains as adapter molecules (Darowski et al. (2019) and disclosed in WO 2018/177966 A1). The cognate CAR, expressed by the engineered T cells, is specific for the above-mentioned mutated Fc variant, containing the previously described P329G mutation. Importantly, the recombinant antibody-based biologics featuring Fc variants with the P329G mutation in combination with the L234A L235A mutations, proved to be essentially Fc effector function silent and non-immunogenic in numerous clinical trials.

In addition to toxicity, another major hurdle in solid tumor CAR-T-therapy is exhaustion and limited persistence of the engineered T cells in patients. For current autologous CAR-T cell therapy, the cells are collected from the patient, engineered and expanded ex vivo with a cytokine cocktail, usually containing Interleukin (IL)-2, but also IL-7, IL-15 and/or IL-21 (Zhang et al. 2020). This expansion step is necessary to achieve high cell numbers, but also leads to terminal T cell differentiation and exhaustion, resulting in limited persistence and modest efficacy. Furthermore, such T cell products are often a mixture of non-engineered and engineered cells, leading to a suboptimal cell composition in the final product. There is a trend towards shortening the ex vivo expansion process in order to limit the cell differentiation and push the cells more towards a stem-like memory T cell phenotype. Reducing ex vivo expansion time and expanding T cells directly in patients would have several benefits, including saving production cost and time and reducing process-mediated differentiation of the T cells. However, until to date, there is no clinically validated procedure established by which CAR-T cells can be specifically expanded in patients.

Conventional IL-2 therapy suffers from dose-limiting systemic side effects mediated by e.g. regulatory T cells or endothelial cells. To overcome this limitation, researchers have developed novel IL-2-based therapies. For example it has been shown that cis-targeting IL-2 to the desired tumor-reactive T cell population has a significantly improved toxicity profile. Different strategies for the cis-targeting have been reported, including e.g., CD8 (Sultan et al. (2021)) or PD1 targeting (Deak et al. (2022)).

### SUMMARY OF THE INVENTION

The invention comprises the combination therapy of a recombinant Fc domain-IL2 variant (Fc-IL2v) polypeptide complex with a membrane-anchored antigen binding (MAB) polypeptide or MAB polypeptide complex for use as a combination therapy, in particular in the treatment of cancer, for the use as a combination therapy in the prevention or treatment of metastasis, or for use as a combination therapy in stimulating an immune response or function, such as T cell activity.

In one aspect, provided is a recombinant Fc domain-IL2 variant (Fc-IL2v) polypeptide complex in combination with a recombinant membrane-anchored antigen binding (MAB) polypeptide or MAB polypeptide complex, for use in the treatment of cancer, for use in the prevention or treatment of metastasis, or for use in stimulating an immune response or function, such as T cell activity,
wherein the recombinant Fc-IL2v polypeptide complex comprises:
   (i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
   (ii) a second polypeptide comprising a variant CH2-CH3 region comprising G329 according to EU numbering, wherein the recombinant Fc-IL2v polypeptide complex does not comprise an antigen binding moiety, and
wherein the MAB polypeptide or MAB polypeptide complex comprises an antigen-binding moiety, or a component thereof, and a transmembrane domain, wherein the antigen-binding moiety binds to a variant CH2-CH3 region comprising the amino acid substitution P329G according to EU numbering relative to the amino acid sequence of a reference CH2-CH3 region comprising P329 according to EU numbering, to which the antigen-binding moiety does not bind.

In one aspect, provided is a recombinant Fc domain-IL2 variant (Fc-IL2v) polypeptide complex, comprising:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
(ii) a second polypeptide comprising a variant CH2-CH3 region comprising G329 according to EU numbering, wherein the recombinant Fc-IL2v polypeptide complex does not comprise an antigen binding moiety.

In one aspect, provided is a method for treatment or prevention of cancer or for stimulating and immune response or function, such as T cell activity in an individual, wherein said method comprises
(a) administration of a recombinant Fc domain-IL2 variant (Fc-IL2v) polypeptide complex to the individual, wherein the recombinant Fc-IL2v polypeptide complex comprises:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
(ii) a second polypeptide comprising a variant CH2-CH3 region comprising G329 according to EU numbering; and
(b) administration of a recombinant membrane-anchored antigen binding (MAB) polypeptide or MAB polypeptide complex, wherein the MAB polypeptide or MAB polypeptide complex comprises an antigen-binding moiety, or a component thereof, and a transmembrane domain, wherein the antigen-binding moiety binds to a variant CH2-CH3 region comprising the amino acid substitution P329G according to EU numbering, relative to the amino acid sequence of a reference CH2-CH3 region comprising P329 according to EU numbering, to which the antigen-binding moiety does not bind.

In one aspect, provided is use of a recombinant Fc domain-IL2 variant (Fc-IL2v) polypeptide complex in the manufacture of a medicament for treatment or prevention of cancer or for stimulating and immune response or function, such as T cell activity in an individual, wherein the recombinant Fc-IL2v polypeptide complex comprises:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
(ii) a second polypeptide comprising a variant CH2-CH3 region comprising G329 according to EU numbering.

In one aspect, provided is use of a recombinant Fc domain-IL2 variant (Fc-IL2v) polypeptide complex in the manufacture of a medicament for the treatment or prevention of cancer or for stimulating and immune response or function, such as T cell activity in an individual, wherein the treatment comprises:
(a) administration of a recombinant Fc domain-IL2 variant (Fc-IL2v) polypeptide complex to the individual, wherein the recombinant Fc-IL2v polypeptide complex comprises:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
(ii) a second polypeptide comprising a variant CH2-CH3 region comprising G329 according to EU numbering; and
(b) administration of a recombinant membrane-anchored antigen binding (MAB) polypeptide or MAB polypeptide complex, wherein the MAB polypeptide or MAB polypeptide complex comprises an antigen-binding moiety, or a component thereof, and a transmembrane domain, wherein the antigen-binding moiety binds to a variant CH2-CH3 region comprising the amino acid substitution P329G according to EU numbering, relative to the amino acid sequence of a reference CH2-CH3 region comprising P329 according to EU numbering, to which the antigen-binding moiety does not bind.

In some aspects, the antigen-binding moiety that binds to the Fc-IL2v comprises the heavy chain variable (VH) region and light chain variable (VL) region of an antibody that binds to the variant CH2-CH3 region comprising the amino acid substitution P329G according to EU numbering.

In some aspects, the antigen-binding moiety is or comprises an Fv, scFv, Fab, Fab′, Fab-SH, F(ab′)₂, crossFab, scFab or dAb moiety.

In some aspects, the antigen-binding moiety comprises:
(a) (i) a VH region incorporating the following CDRs:
   HC-CDR1 having the amino acid sequence of SEQ ID NO:11;
   HC-CDR2 having the amino acid sequence of SEQ ID NO: 19; and
   HC-CDR3 having the amino acid sequence of SEQ ID NO: 13;
   and
   (ii) a VL region incorporating the following CDRs:
   LC-CDR1 having the amino acid sequence of SEQ ID NO:24;
   LC-CDR2 having the amino acid sequence of SEQ ID NO:25; and
   LC-CDR3 having the amino acid sequence of SEQ ID NO:26;
or
(b) (i) a VH region incorporating the following CDRs:
   HC-CDR1 having the amino acid sequence of SEQ ID NO:11;
   HC-CDR2 having the amino acid sequence of SEQ ID NO: 12; and
   HC-CDR3 having the amino acid sequence of SEQ ID NO: 13;
   and
   (ii) a VL region incorporating the following CDRs:
   LC-CDR1 having the amino acid sequence of SEQ ID NO:24;
   LC-CDR2 having the amino acid sequence of SEQ ID NO:25; and
   LC-CDR3 having the amino acid sequence of SEQ ID NO:26.

In some aspects, the mutant IL-2 polypeptide further comprises the amino acid substitution Q126T.

In some aspects, the recombinant Fc-IL2v polypeptide complex does not comprise an antigen binding moiety, in particular wherein the recombinant Fc-IL2v polypeptide complex does not comprise a scFv, Fab or crossFab.

In some aspects, the recombinant MAB polypeptide comprises an amino acid sequence derived from IL2Ra, IL15Ra or CD8a.

In some aspects, the recombinant MAP polypeptide is a chimeric antigen receptor (CAR).

In some aspects, the recombinant MAP polypeptide comprises at least one recombinant CD3-TCR complex polypeptide.

In some aspects, the recombinant CD3-TCR complex polypeptide comprises:
(i) an antigen-binding moiety, or a component thereof, wherein the antigen-binding moiety binds to the variant CH2-CH3 region comprising the amino acid substitution P329G according to EU numbering, relative to the amino acid sequence of a reference CH2-CH3 region comprising P329 according to EU numbering, to which the antigen-binding moiety does not bind; and
(ii) a CD3-TCR complex association domain having an amino acid sequence derived from a CD3-TCR complex polypeptide.

In some aspects, the recombinant CD3-TCR complex polypeptide is capable of associating through its CD3-TCR complex association domain with one or more CD3-TCR complex polypeptides to form a CD3-TCR complex.

In some aspects, the amino acid sequence derived from a CD3-TCR complex polypeptide is derived from CD3ε, TCRα or TCRβ.

In some aspects, provided is a cell comprising a recombinant MAB polypeptide or MAB polypeptide complex as hereinbefore described.

In some aspects, provided is a method of producing an enriched pool of cells comprising contacting a starting pool of cells comprising at least one cell as hereinbefore described with the recombinant Fc-IL2v polypeptide complex as hereinbefore described and incubating the cells until the fraction of cells comprising the recombinant MAB polypeptide or MAB polypeptide complex reaches a desired fraction of the total pool of cells to produce the enriched pool of cells.

In some aspects, provided is a nucleic acid, or a plurality of nucleic acids, encoding a recombinant Fc domain-IL2 variant (Fc-IL2v) polypeptide complex as hereinbefore described, or a recombinant MAB polypeptide or MAB polypeptide complex as hereinbefore described.

In some aspects, provided is an expression vector, or a plurality of expression vectors, comprising a nucleic acid as hereinbefore described.

In some aspects, provided is a cell comprising a recombinant MAB polypeptide or MAB polypeptide complex as hereinbefore described, a nucleic acid or a plurality of nucleic acids as hereinbefore described, or an expression vector or a plurality of expression vectors as hereinbefore described.

In some aspects, cells expressing the recombinant MAB polypeptide and/or the recombinant MAB polypeptide complex cells are specifically expanded, in particular wherein the cells are specifically expanded by contacting the cells with the recombinant Fc-IL2v polypeptide complex as hereinbefore described.

In some aspects, cells expressing the recombinant MAB polypeptide and/or the recombinant MAB polypeptide complex cells are enriched, in particular wherein the cells are enriched by contacting the cells with the recombinant Fc-IL2v polypeptide complex as hereinbefore described.

In some aspects, cells expressing the recombinant MAB polypeptide and/or the recombinant MAB polypeptide complex cells are enriched to >90% of a total cell pool.

In some aspects, provided is a method of producing an enriched pool of cells comprising contacting a starting pool of cells comprising at least one cell as hereinbefore described with the recombinant Fc-IL2v polypeptide complex as hereinbefore described and incubating the cells until the fraction of cells comprising the recombinant MAB polypeptide or MAB polypeptide complex reaches a desired fraction of the total pool of cells to produce the enriched pool of cells.

In some aspects, provided is a pharmaceutical composition comprising a recombinant Fc domain-IL2 variant (Fc-IL2v) polypeptide complex as hereinbefore described, a cell as hereinbefore described or an enriched pool of cells produced as hereinbefore described.

In some aspects, provided is the invention as hereinbefore described with reference to the Figures and Examples.

### BRIEF DESCRIPTION OF THE FIGURES

FIG. 1A-1D. Schematic representation of exemplary recombinant membrane-anchored antigen bining (MAB) polypeptides and MAB polypeptide complexes according to the present disclosure. Depicted are (from left to right) a recombinant CD38-TCR complex polypeptide in a TCR complex, a recombinant TCRαβ based MAB polypeptide complex, recombinant first or second generation CARs and recombinant non-signaling tags with a binding moiety in form of a scFv (1A). Schematic representation of the gene constructs corresponding to the P329G-CARs (1B). TCR-based MAB polypeptide complexes (P329G-CD3ε and P329G-Cαβ) (1C) and non-signaling MAB polypeptide (P329G-tag, 1D).

FIG. 2. Schematic representation of exemplary recombinant Fc-IL2v polypeptide complexes according to the present disclosure. An IL2 variant is fused via a linker to a variant Fc containing an orthogonal mutation resulting in an orthogonal ligand to the MAB polypeptide or MAB polypeptide complex according to the present disclosure.

FIG. 3. Schematic representation of exemplary recombinant Fc-IL2v polypeptide complexes according to the present disclosure and used in the Examples. Fc_P329G_LALA conjugated IL2v (Q126T) and PD1-IL2v (Q126T) (also containing the P329G_LALA mutations) served as orthogonal ligands, while the Fc_WT or Fc_LALA conjugated molecules served as non targeting controls.

FIGS. 4A-4C. Schematic representation of P329G-CAR and P329G-CD3ε/Cαβ constructs. FIG. 4A depicts a second generation chimeric antigen receptor (CAR) with the anti-P3290 binding moiety in the scFv format. FIGS. 4B and 4C show the P329G-CD3ε/P329G-Cαβ constructs in the context of the endogenous TCR complex. The anti-P329G scFv is either fused to the CD3ε chain (4B, P329G-CD3ε TCR complex) or the VH was fused to the Ca TCR domain and the VL fused to the Cβ TCR domain (4C, P329G-Cαβ TCR complex). The P329G-Cαβ construct can be further stabilized by introducing an interchain disulfide bond between the Cα and Cβ extracellular domains.

FIGS. 5A and 5B. Staining of Jurkat NFAT (TCR/CD3 Effector Cells (NFAT), Promega, #J1601) wildtype (wt) or Jurkat NFAT after CRISPR-Cas9 knock-out of endogenous CD3ε with anti-CD3ε-FITC (1:50, Biolegend, #300406) antibody. FIG. 6A depicts the staining after the knock-out, with Jurkat NFAT wildtype cells as control. FIG. 6B shows the population before and after sorting for CD3ε negative cells, leading to a 99.7% CD3ε negative population.

FIGS. 6A and 6B, eGFP expression in Jurkat NFAT CD3ε KO cells after lentiviral transduction of P329G-CD3ε (6A) or P329G-CAR (6B) and pool sorting for living, eGFP positive cells. As negative control served mock transduced cells (cells transduced with empty virus-like particles (VLPs)).

FIGS. 7A-7C: Surface expression of P329G-CAR or P329G-CD3ε TOR in Jurkat NFAT CD3ε KO cells (sorted pools) was confirmed by staining with AF647 labeled Fc-P329G LALA as illustrated in 7A (1) with the corresponding staining histograms depicted in 7B (1) The integration into the TCR complex and its expression on the cell surface was assessed by staining with anti-TCRαβ-BV421 (1:50, Biolegend, #306722) and anti-CD3ε-PE (1:50, Biolegend, #300408) antibodies (7A (2, 3)). The corresponding stainings are shown in FIG. 7B (2, 3) and FIG. 7C (2, 3) As negative control for the stainings served mock transduced cells (light gray).

FIGS. 8A and 8B. Activation of Jurkat NFAT CD3ε KO cells transduced with P329G-CAR (sorted pool) or P329G-CD3ε (sorted pool) in the presence of FolR1′ target cells with high (HeLa) or low (HT-29) target expression levels upon stimulation with anti-FolR1 (clone 16D5) IgG containing the P329G LALA mutations. Activation was assessed by quantification of the intensity of TCR/CD3 downstream signaling reported by NFAT promoter-controlled luciferase expression Schematic representation of the assay (8A). Dose-dependent activation of transduced Jurkat cells in the presence of HT29 or HeLa (8B) as target cells. Depicted are technical average values from triplicates, error bars indicate SD.

FIGS. 9A and 9B. Activation of Jurkat NFAT CD3ε KO cells transduced with P329G-CAR (sorted pool) or P329G-CD3ε (sorted pool) in the presence of CD19⁺ target cells with high (Nalm-6) or low (Z138) target expression levels upon stimulation with anti-CD19 (affinity maturated 2B11) IgG containing the P329G LALA mutations. Activation was assessed by quantification of the intensity of TCR/CD3 downstream signaling reported by NFAT promoter-controlled luciferase expression. Schematic representation of the assay (9A). Dose-dependent activation of transduced Jurkat cells in the presence of Z138 or Nalm-6 (9B) as target cells. Depicted are technical average values from triplicates, error bars indicate SD.

FIGS. 10A and 10B: eGFP expression in Jurkat TCRαβ KO-CD4+ cells (T Cell Activation Bioassay (TCRαβ-KO), Promega, #GA1172) after lentiviral transduction of P329G-Cαβ (10A) or P329G-CAR (10B) and pool sorting for living, eGFP positive cells. As negative control served mock transduced cells.

FIGS. 11A-11C: Surface expression of the P329G-Cαβ TCR or P329G-CAR on Jurkat TCRαβ KO-CD4+ cells (sorted pool) was checked by staining with an IgG containing the P3290 LALA mutation (anti-FolR1 IgG P329G LALA) and detection of binding by secondary PE-F(ab)2 fragment anti-huIgG (F(ab)2 fragment specific) (Jackson ImmunoResearch, #109-116-097) (11A (1)). The incorporation of the VH-TCRα and VL-TCRβ chains was confirmed by staining with anti-TCRαβ-BV421 (1:50, Biolegend, #306722) and anti-CD3ε-APC (1:50, Biolegend, #300412) antibodies (11A (2,3)). The corresponding staining results are shown in FIG. 11B (1, 2, 3) and FIG. 11C (1, 2, 3). For all stainings (1, 2, 3) the staining of mock transduced cells served as negative control (light gray). As additional negative control for the P329G staining (1) the transduced cells were also stained with secondary antibody only (staining overlaying with mock transduced control (light gray)).

FIGS. 12A and 12B. Activation of Jurkat TCRαβ KO-CD4+ cells transduced with P329G-Cαβ of P329G-CAR (sorted pool) in the presence of FolR1⁺ target cells with high (HeLa) or low (HT-29) target expression levels upon stimulation with anti-FolR1 (clone 16D5) IgG containing the P329G LALA mutation Activation was assessed by quantification of the intensity of TCR/CD3 downstream signaling reported by IL2 promoter-controlled luciferase expression. Schematic representation of the assay (12A). Dose-dependent activation of transduced Jurkat cells in the presence of HT29 or HeLa (12B) as target cells. Depicted are technical average values from triplicates, error bars indicate SD.

FIGS. 13A and 13B. Activation of Jurkat TCRαβ KO−CD4+ cells transduced with P329G-Cαβ or P329G-CAR (sorted pool) in the presence of CD19⁺ target cells with high (Nalm-6) or low (Z138) target expression levels upon stimulation with anti-CD 19 (affinity maturated 2B11) IgG containing the P329G LALA mutation. Activation was assessed by quantification of the intensity of TCR/CD3 downstream signaling reported by IL2 promoter-controlled luciferase expression. Schematic representation of the assay (13A). Dose-dependent activation of transduced Jurkat cells in the presence of Z138 or Nalm-6 (13B) as target cells. Depicted are technical average values from triplicates, error bars indicate SD.

FIG. 14A-14C. Human Pan T cells of two donors were transduced with P329G-CAR, P329G-Cαβ or P329G-CD3ε respectively. In the case of the P329G-Cαβ construct the endogenous TCRα and TCRβ chains and in the case of the P329G-CD3& construct the endogenous CD3ε were knocked-out using CRISPR-Cas9. (14A) shows the eGFP expression after transduction and knock-out of the respective endogenous TCR chains in both donors. The surface expression of the different constructs was determined by staining with AF647 labeled Fc-P329G LALA (14B). Staining with anti-CD3ε-PE (1:50, Biolegend, #300408) and Fc-P329G LALA-AF647 to check the percentage of correctly assembled P329G-CD3ε TCR or P329G-Cαβ TCR complexes is shown in (14C).

FIG. 15. eGFP expression in CTLL-2 cells after lentiviral transduction with a second generation P329G-CAR (4-1BB). Surface expression of the P329G-CAR (4-1BB) was confirmed by staining with AF647 labeled Fc_P329G_LALA, showing that ˜94% of the cells are expressing the receptor.

FIG. 16. Proliferation of CTLL-2 cells expressing the P329G-CAR (4-1BB) incubated with Fc_P329G_LALA-IL2v/IL2v-Fc_P329G_LALA or Fc_LALA-IL2v/IL2v-Fc_LALA or Proleukin. After 72 hours of incubation, the number of cells was quantified in a CellTiter-Glo viability assay. Targeting of IL2v to the cells via P329G mutation leads to proliferation even at low concentrations of the Fc_P329G_LALA fused cytokine.

FIG. 17. eGFP expression in primary T cells after lentiviral transduction with a second generation P329G-CAR (CD28). Surface expression of the P329G-CAR (CD28) was confirmed by staining with AF647 labeled Fc_P329G_LALA, showing that ˜70% of the cells are expressing the receptor.

FIG. 18. STAT5 phosphorylation (pSTAT5) of primary T cells transduced with P329G-CAR (CD28) after stimulation with Fc_P329G_LALA-IL2vQ126T or Fc_WT-IL2vQ126T. Shown is the pSTAT5 median fluorescence intensity after gating on eGFP+ (P329G-CAR+) or eGFP− (P329G-CAR−) cells. Targeting of Fc_P329G_LALA-IL2vQ126T via the P329G mutation to P329G-CAR T cells leads to a ˜230-fold difference in the EC50 compared to the effect of Fc_WT-IL2vQ126T on the same population. Depicted are technical average values from duplicates, error bars indicate SD. The EC50 values were calculated with GraphPad Prism 8.4.2 (log (agonist) vs. response—Variable slope (four parameters)).

FIG. 19. STAT5 phosphorylation of primary T cells transduced with P329G-CAR (CD28) after stimulation with PD1-IL2v or PD1-IL2vQ126T. Shown is the pSTAT5 median fluorescence intensity after gating on eGFP+ (P329G-CAR+) or eGFP− (P329G-CAR−) cells. Further attenuation of IL2v by the Q126T mutation leads to an increase of the cis-effect by the P329G mutation (˜100× vs. ˜800×). Depicted are technical average values from duplicates, error bars indicate SD. The EC50 values were calculated with GraphPad Prism 8.4.2 (log (agonist) vs. response—Variable slope (four parameters)).

FIG. 20. eGFP expression in primary T cells after lentiviral transduction with different P329G-tags (CD8a, CD25 or IL15Ra membrane anchor-based). Surface expression of the P329G-tags were confirmed by staining with AF647 labeled Fc_P329G_LALA. Depending on the membrane anchor used, between 47%-75% of the cells were expressing the respective P329G tag constructs on the cell surface.

FIG. 21. STAT5 phosphorylation of primary T cells transduced with different P329G-tags after stimulation with Fc_P329G_LALA-IL2v or Fc_LALA-IL2v. Shown is the pSTAT5 median fluorescence intensity after gating on eGFP+ (P329G-tag+) or eGFP− (P329G-tag−) cells. The P329G-tag with the IL15Ra membrane anchor showed the best cis-targeting window (˜185-fold). Depicted are technical average values from duplicates, error bars indicate SD. The data was analyzed with GraphPad Prism 8.4.2 (log (agonist) vs. response—Variable slope (four parameters)).

FIG. 22. STAT5 phosphorylation of primary T cells transduced with different P329G-tags after stimulation with PD1-IL2v or PD1-IL2vQ126T. Shown is the pSTAT5 median fluorescence intensity after gating on eGFP+ (P329G-tag+) or eGFP− (P329G-tag−) cells. The P329G-tag with the IL15Ra membrane anchor again showed the best cis-targeting window (˜75-fold). Depicted are technical average values from duplicates, error bars indicate SD. The data was analyzed with GraphPad Prism 8.4.2 (log (agonist) vs. response—Variable slope (four parameters)).

FIG. 23. CellTrace violet proliferation assay of primary T cells expressing the P329G-tag (IL15Ra) incubated with Fc_LALA-IL2v (23A), Fc_P329G_LALA-IL2v (23B) or PD1-IL2v (C). The cells were incubated for 5 days with the compounds and the proliferation of eGFP+ and eGFP-cells was assessed by flow cytometry by analyzing the decrease of CellTrace violet dye in the dividing population (23A, 23B and 23C). The cells were stained with AF647 labeled Fc_P329G_LALA and checked for eGFP expression after 6 days of expansion and the population shifted to >90% eGFP+ (23D) P329G-tag expressing cells (23E).

FIG. 24. eGFP expression in primary T cells of two donors after CRISPR KO of endogenous CD3ε and lentiviral transduction with P329G-CD38. KO efficiency and surface expression of the P329G-CD3ε were checked by staining with AF647 labeled Fc_P329G_LALA and PE anti-CD38, showing that the KO was ˜98% successful and ˜34-40% of the cells were expressing the P329G-CD3ε.

FIG. 25. STAT5 phosphorylation of primary T cells (donor 8) transduced with P329G-CD3ε after stimulation with Fc_P329G_LALA-IL2v or Fc_LALA-IL2v. Shown is the pSTAT5 median fluorescence intensity after gating on eGFP+ (P329G-CD3ε+) or eGFP− (P329G-CD3ε−) cells. Depicted are technical average values from duplicates, error bars indicate SD. The EC50 values were calculated with GraphPad Prism 8.4.2 (log (agonist) vs. response—Variable slope (four parameters)).

FIG. 26. STAT5 phosphorylation of primary T cells (donor 8) transduced with P329G-CD3ε after stimulation with Fc_P329G_LALA-IL2vQ126T or Fc_WT-IL2vQ126T. Shown is the pSTAT5 median fluorescence intensity after gating on eGFP+ (P329G-CD3ε+) or eGFP− (P329G-CD3ε−) cells. Compared to Fc_P329G_LALA-IL2v the Fc_P329G_LALA-IL2vQ126T lead to an increased cis-targeting window of ˜90-fold. Depicted are technical average values from duplicates, error bars indicate SD. The EC50 values were calculated with GraphPad Prism 8.4.2 (log (agonist) vs. response—Variable slope (four parameters)).

FIG. 27. CellTrace violet proliferation assay of primary T cells (donor 7) expressing the P329G-CD3ε TCR incubated with Fc_LALA-IL2v (28A) or Fc_P329G_LALA-IL2v (28B). The cells were incubated for 5 days with the compounds and the proliferation of eGFP+ and eGFP-cells was assessed by flow cytometry by analyzing the decrease of CellTrace violet dye in the dividing population. The cells were stained with AF647 labeled Fc_P329G_LALA and checked for eGFP expression after 5 days of expansion and the population shifted to ˜82% eGFP+ or ˜75% P329G-CD3ε expressing cells (28C and 28D).

FIG. 28. eGFP expression in primary T cells after lentiviral transduction with different P329G-receptors (P329G-CAR, P329G-tag, P329G-CD3ε, P329G-Cαβ). Surface expression of the P329G-receptors was confirmed by staining with AF647 labeled Fc_P329G_LALA. Depending on the construct, between 59%-83% of the cells were expressing the receptor on the cell surface (28A). KO efficiency and surface expression of the P329G-CD3ε and P329G-Cαβ were assessed by staining with AF647 labeled Fc_P329G_LALA and PE anti-CD3ε or BV421 anti-TCRαβ. In the case of P329G-CD3ε the KO was ˜98% successful and ˜68% of the cells were expressing the P329G-CD38. The P329G-Cαβ T cells showed ˜62% of correctly formed TCR complexes on the surface (28B). PD1 expression was assessed on the day the pSTAT5 assay was performed (day 12 after transduction) (28C) and after reactivation of the cells with ImmunoCult™ Human CD3/CD28/CD2 T Cell Activator (28D). Shown is the PD1 expression of the P329G-tag (IL15Rα) cells.

FIG. 29. STAT5 phosphorylation of primary T cells transduced with P329G-CAR (CD28) after stimulation with Fc_P329G_LALA-IL2v, Fc_LALA-IL2v, IL2-Fc_P329G_LALA, IL2-Fc_LALA, Fc_P329G_LALA-IL2vQ126T, Fc_WT-IL2vQ126T, PD1-IL2v or PD1-IL2vQ126T. Shown is the pSTAT5 median fluorescence intensity after gating on eGFP+ (P329G-CAR+) or eGFP− (P329G-CAR−) cells (29A). The graphs were rearranged to allow a direct comparison of N-terminal vs. C-terminal Fc-IL2v fusion, PD1-IL2v vs. Fc_P329G_LALA-IL2v and Fc-fused IL2v vs. IL2vQ126T (29B). Depicted are technical average values from duplicates, error bars indicate SD. The EC50 values were calculated with GraphPad Prism 8.4.2 (log (agonist) vs. response—Variable slope (four parameters)).

FIG. 30. STAT5 phosphorylation of primary T cells transduced with P329G-tag (IL15Rα) after stimulation with Fc_P329G_LALA-IL2v, Fc_LALA-IL2v, IL2-Fc_P329G_LALA, IL2-Fc_LALA, Fc_P329G_LALA-IL2vQ126T, Fc_WT-IL2vQ126T, PD1-IL2v or PD1-IL2vQ126T. Shown is the pSTAT5 median fluorescence intensity after gating on eGFP+ (P329G-tag+) or eGFP− (P329G-tag−) cells (30A). The graphs were rearranged to allow a direct comparison of N-terminal vs. C-terminal Fc-IL2v fusion, PD1-IL2v vs. Fc_P329G_LALA-IL2v and Fc-fused IL2v vs. IL2vQ126T (30B). Depicted are technical average values from duplicates, error bars indicate SD. The EC50 values were calculated with GraphPad Prism 8.4.2 (log (agonist) vs. response—Variable slope (four parameters)).

FIG. 31. STAT5 phosphorylation of primary T cells transduced with P329G-CD3ε after stimulation with Fc_P329G_LALA-IL2v, Fc_LALA-IL2v, IL2-Fc_P329G_LALA, IL2-Fc_LALA, Fc_P329G_LALA-IL2vQ126T, Fc_WT-IL2vQ126T, PD1-IL2v or PD1-IL2vQ126T. Shown is the pSTAT5 median fluorescence intensity after gating on eGFP+ (P329G-CD3ε+) or eGFP− (P329G-CD3ε−) cells (31A). The graphs were rearranged to allow a direct comparison of N-terminal vs. C-terminal Fc-IL2v fusion, PD1-IL2v vs. Fc_P329G_LALA-IL2v and Fc-fused IL2v vs. IL2vQ126T (31B). Depicted are technical average values from duplicates, error bars indicate SD. The EC50 values were calculated with GraphPad Prism 8.4.2 (log (agonist) vs. response—Variable slope (four parameters)).

FIG. 32. STAT5 phosphorylation of primary T cells transduced with P329G-Cαβ after stimulation with Fc_P329G_LALA-IL2v, Fc_LALA-IL2v, IL2-Fc_P329G_LALA, IL2-Fc_LALA, Fc_P329G_LALA-IL2vQ126T, Fc_WT-IL2vQ126T, PD1-IL2v or PD1-IL2vQ126T. Shown is the pSTAT5 median fluorescence intensity after gating on eGFP+ (P329G-Cαβ+) or eGFP− (P329G-Cαβ−) cells (32A). The graphs were rearranged to allow a direct comparison of N-terminal vs. C-terminal Fc-IL2v fusion, PD1-IL2v vs. Fc_P329G_LALA-IL2v and Fc-fused IL2v vs. IL2vQ126T (32B). Depicted are technical average values from duplicates, error bars indicate SD. The EC50 values were calculated with GraphPad Prism 8.4.2 (log (agonist) vs. response—Variable slope (four parameters)).

FIG. 33. Primary T cells transduced with P329G-CD3ε and endogenous CD3ε knockout, expanded with either T cell medium with IL2 (50 IU/ml), IL7 (25 ng/ml) and IL 15 (50 ng/ml) or 0.5 nM Fc_P329G_LALA-IL2v. After 6 days the population shifted from 37% P329G-CD3ε cells to 87% of the desired population (33A). The cells were then directly compared in a Incucyte killing assay with two tumor cell lines (HeLa NLR and MKN45 NLR) (33B). The T cell number was adjusted to 10,000 eGFP+ cells-to 10.000 target cells per well (E:T 1:1) in order to allow a fair comparison. Looking at the red cell count over time, the selectively expanded P329G-CD3ε T cells are fully functional and allow killing of the target cells comparable to the IL2, IL7, IL15 expanded P329G-CD3ε T cells. This suggests that the P329G-receptor is free for binding the adapter IgG and not blocked by the Fc_P329G_LALA-IL2v. Depicted are technical average values from duplicates, error bars indicate SD.

FIG. 34: Schematic representation of the orthogonal ligands PD1-IL2v, PD1-reg-IL2v and one-armed (OA)-PD1-reg-IL2v and their binding to the GFP+ (P329G-tag+) cells vs GFP− (P329G-tag−) PD-1^{low} cells.

FIG. 35: eGFP expression and surface staining (AF647 labeled Fc_P329G_LALA) of the P329G-tag (IL15Ra based) in primary T cells after lentiviral transduction. 63.9% of the cells were expressing GFP and the P329G-tag construct on the cell surface. PD-1 expression was stained (PD-1-PE) shortly before the pSTAT5 assay was performed. Compared to the isotype control. 4.87% of the T cells were PD-1 positive (35A). STAT5 phosphorylation was checked after stimulation with PD1-IL2v, PD1-reg-IL2v and OA-PD1-reg-IL2v. Shown is the pSTAT5 median fluorescence intensity after gating on eGFP+ (P329G-tag+) or eGFP− (P329G-tag−) cells (35B).

FIG. 36: Schematic representation of the orthogonal ligands PD1-IL2v. PD1-reg-IL2v and one-armed (OA)-PD1-reg-IL2v and their binding to the GFP+ (P329G-tag+) cells vs GFP− (P329G-tag−) PD-1^{high} cells.

FIG. 37: eGFP expression and surface staining (AF647 labeled Fc_P329G_LALA) of the P329G-tag (IL15Ra based) in primary T cells after lentiviral transduction. 60.9% of the cells were expressing GFP and the P329G-tag construct on the cell surface. For one part of the assay the transduced T cells were reactivated using Dynabeads Human T-Activator CD3/CD28. For the other part of the assay the cells were not reactivated. PD-1 expression was stained (PD-1-PE) shortly before the pSTAT5 assay was performed. Compared to the isotype control, in the not reactivated condition 14.7% of the T cells were PD-1 positive (37A), while 81.6% of the T cells were PD-1 positive in the reactivated condition (37C). STAT5 phosphorylation was checked after stimulation with PD1-IL2v, PD1-reg-IL2v and OA-PD1-reg-IL2v. Shown is the pSTAT5 median fluorescence intensity after gating on eGFP+ (P329G-tag+) or eGFP− (P329G-tag−) cells in the PD-1^{low} (B) and PD-1^{high} conditions (37D).

### DETAILED DESCRIPTION OF THE INVENTION

The present inventors have generated a new orthogonal cis-targeting method allowing to selectively expand engineered T cells, based on membrane-anchored antigen binding (MAB) polypeptides comprising an antigen binding moiety capable of specific binding to a CH2-CH3 region comprising the amino acid substitution P329G according to EU numbering. Exemplary embodiments include but are not limited to chimeric antigen receptors (CARs). TCR-based MAB polypeptides or non-signaling tag-like MAB polypeptides. In some aspects, recombinant Fc domain-IL2 variant (Fc-IL2v) polypeptide complexes comprising a CH2-CH3 region comprising the amino acid substitution P329G according to EU numbering fused to IL-2 or variants thereof are provided. Since naturally-occuring Fc domains do not comprise this P329G mutation, the recombinant Fc-IL2v polypeptide represent an orthogonal cytokine ligand for engineered (T) cells expressing a recombinant MAB polypeptide or recombinant MAB polypeptide complex according to the present disclosure. The recombinant Fc-IL2v polypeptide complex according to the present invention can be used for the specific expansion of MAB polypeptide (complex) expressing T cells and specific enrichment of such cells, resulting in a reduced heterogeneity of the final cell product. The technology described here can be translated to a safe, specific and controllable CAR-T cell expansion in patients in the future and improve the therapeutic outcome of cell therapies for different cancer indications including solid tumors.

In some aspects, the present disclosure provides recombinant Fc domain-IL2 variant (Fc-IL2v) polypeptide complexes, and such Fc-IL2v polypeptide complexes in combination with a recombinant membrane-anchored antigen binding (MAB) polypeptide for use as a combination therapy in the treatment of cancer, for use as a combination therapy in the prevention or treatment of metastasis, or for use as a combination therapy in stimulating an immune response or function, such as T cell activity. The present disclosure further provides nucleic acids and vectors encoding such Fc-IL2v polypeptide complexes and/or recombinant MAB polypeptides, cell comprising such Fc-IL2v polypeptide complexes and/or recombinant MAB polypeptides, and compositions comprising such Fc-IL2v polypeptide complexes, recombinant MAB polypeptides, and/or cells.

More specifically, the present disclosure is directed to a novel recombinant Fc-IL2v polypeptide complex, comprising variant CH2-CH3 polypeptides and an IL-2 variant polypeptide. The present disclosure is further directed to recombinant MAB polypeptides and MAB polypeptide complexes comprising an antigen-binding moiety and a transmembrane domain, wherein the antigen-binding moiety specifically binds to the variant CH2-CH3 region of the Fc-IL2 polypeptide complex. Thus, cells (e.g. T cells) comprising the recombinant MAB polypeptide(s) specifically bind to the recombinant Fc-IL2v polypeptide complex and become activated.

Unexpectedly, the present disclosure demonstrates that the recombinant Fc-IL2v polypeptide complexes are capable of triggering strong IL2 receptor-mediated signaling of cells (e.g. T cells) comprising (e.g. through expression) the MAB polypeptide and/or MAB polypeptide complexes.

More unexpectedly still, in experiments providing for direct comparison of the level of activation of T cells contacted with the novel Fc-IL2v polypeptide complex and expressing the MAB polypeptide and/or MAB polypeptide complexes. T cells are shown to be activated to a greater extent by the novel Fc-IL2v polypeptide complex which does not comprise any further antigen binding moiety.

Terms are used herein as generally used in the art, unless otherwise defined in the following.

### Therapeutic Methods and Compositions

In some aspects, the invention comprises a method for the treatment of a patient in need of therapy, characterized by administering to the patient a therapeutically effective amount of the combination therapy of a Fc-IL2v polypeptide complex as herein described with (cells expressing) a recombinant MAB polypeptide and/or a recombinant MAB polypeptide complex as herein described.

Further provided is the use of a Fc-IL2v polypeptide complex as herein described with (cells expressing) a recombinant MAB polypeptide and/or a recombinant MAB polypeptide complex as herein described for the described combination therapy.

One preferred embodiment of the invention is the combination therapy of a Fc-IL2v polypeptide complex as herein described with (cells expressing) a recombinant MAB polypeptide and/or a recombinant MAB polypeptide complex as herein described for use in the treatment of cancer or tumor.

Thus one embodiment of the invention is a Fc-IL2v polypeptide complex as herein described for use in the treatment of cancer or tumor in combination with (cells expressing) a recombinant MAB polypeptide and/or a recombinant MAB polypeptide complex as herein described.

A further embodiment of the invention is (cells expressing) a recombinant MAB polypeptide and/or a MAB polypeptide complex as herein described for use in the treatment of cancer of tumor in combination with a Fc-IL2v polypeptide complex as herein described.

In some aspects the recombinant Fc-IL2v polypeptide complex, recombinant MAB polypeptide and/or MAB polypeptide complex, method or use as described herein further comprises administration of a targeting antibody (comprising G329 in the Fc domain according to EU numbering) as further described herein below.

In some aspects the cancer or tumor may present an antigen, e.g. FolR1, CEA or CD19. In further aspects, the cancer or tumor may present an antigen in a tumor cell environment, e.g. on PD-1+ T cells. PD-1 as the target of the combination therapy may be presented in the tumor cell environment, e.g. in PD-1+ T cells. The treatment may be of a solid tumor. The treatment may be of a carcinoma. The cancer may be selected from the group consisting of colorectal cancer, head and neck cancer, non-small cell lung cancer, breast cancer, pancreatic cancer, liver cancer and gastric cancer. The cancer may be selected from the group consisting of lung cancer, colon cancer, gastric cancer, breast cancer, head and neck cancer, skin cancer, liver cancer, kidney cancer, prostate cancer, pancreatic cancer, brain cancer and cancer of the skeletal muscle.

The term “cancer” as used herein may be, for example, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, lymphocytic leukemia, including refractory versions of any of the above cancers, or a combination of one or more of the above cancers. In one preferred embodiment such cancer is a breast cancer, colorectal cancer, melanoma, head and neck cancer, lung cancer or prostate cancer. In one preferred embodiment such cancer is a breast cancer, ovarian cancer, cervical cancer, lung cancer or prostate cancer. In another preferred embodiment such cancer is breast cancer, lung cancer, colon cancer, ovarian cancer, melanoma cancer, bladder cancer, renal cancer, kidney cancer, liver cancer, head and neck cancer, colorectal cancer, pancreatic cancer, gastric carcinoma cancer, esophageal cancer, mesothelioma, prostate cancer, leukemia, lymphoma, myelomas. In a preferred embodiment such cancer is a FolR1, CEA and/or CD19-expressing cancer.

An embodiment of the invention is a Fc-IL2v polypeptide complex as described herein in combination with (cells expressing) a recombinant MAB polypeptide and/or a recombinant MAB polypeptide complex as described herein and optionally a targeting antibody (comprising G329 in the Fc domain according to EU numbering) as described herein for use in the treatment of any of the above described cancers or tumors. Another embodiment of the invention is (cells expressing) a recombinant MAB polypeptide and/or a recombinant MAB polypeptide complex as described herein in combination with a Fc-IL2v polypeptide complex as described herein and optionally a targeting antibody (comprising G329 in the Fc domain according to EU numbering) as described herein for use in the treatment of any of the above described cancers or tumors.

The invention comprises the combination therapy with a Fc-IL2v polypeptide complex as described herein with (cells expressing) a recombinant MAB polypeptide and/or recombinant MAB polypeptide complex and optionally a targeting antibody (comprising G329 in the Fc domain according to EU numbering) as described herein for the treatment of cancer.

The invention comprises the combination therapy with a Fc-IL2v polypeptide complex as described herein with a (cells expressing) a recombinant MAB polypeptide and/or recombinant MAB polypeptide complex and optionally a targeting antibody (comprising G329 in the Fc domain according to EU numbering) as described herein for the prevention or treatment of metastasis.

The invention comprises the combination therapy of a Fc-IL2v polypeptide complex as described herein with (cells expressing) a recombinant MAB polypeptide and/or a recombinant MAB complex as described herein and optionally a targeting antibody (comprising G329 in the Fc domain according to EU numbering) as described herein for use in stimulating an immune response or function, such as T cell activity.

The invention comprises a method for the treatment of cancer in a patient in need thereof, characterized by administering to the patient a Fc-IL2v polypeptide complex as described herein and (cells expressing) a recombinant MAB polypeptide and/or a recombinant MAB complex as described herein and optionally a targeting antibody (comprising G329 in the Fc domain according to EU numbering) as described herein.

The invention comprises a method for the prevention or treatment of metastasis in a patient in need thereof, characterized by administering to the patient a Fc-IL2v polypeptide complex as described herein and (cells expressing) a recombinant MAB polypeptide and/or a recombinant MAB complex as described herein and optionally a targeting antibody (comprising G329 in the Fc domain according to EU numbering) as described herein.

The invention comprises a method for stimulating an immune response or function, such as T cell activity, in a patient in need thereof, characterized by administering to the patient a Fc-IL2v polypeptide complex as described herein and (cells expressing) a recombinant MAB polypeptide and/or a recombinant MAB complex as described herein and optionally a targeting antibody (comprising G329 in the Fc domain according to EU numbering) as described herein.

The invention comprises a Fc-IL2v polypeptide complex as described herein for use in the treatment of cancer in combination with (cells expressing) a recombinant MAB polypeptide and/or a recombinant MAB complex as described herein and optionally a targeting antibody (comprising G329 in the Fc domain according to EU numbering) as described herein, or alternatively for the manufacture of a medicament for the treatment of cancer in combination with (cells expressing) a recombinant MAB polypeptide and/or a recombinant MAB complex as described herein and optionally a targeting antibody (comprising G329 in the Fc domain according to EU numbering) as described herein.

The invention comprises a Fc-IL2v polypeptide complex as described herein for use in the prevention or treatment of metastasis in combination with (cells expressing) a recombinant MAB polypeptide and/or a recombinant MAB complex as described herein and optionally a targeting antibody (comprising G329 in the Fc domain according to EU numbering) as described herein, or alternatively for the manufacture of a medicament for the prevention or treatment of metastasis in combination with (cells expressing) a recombinant MAB polypeptide and/or a recombinant MAB complex as described herein and optionally a targeting antibody (comprising G329 in the Fc domain according to EU numbering) as described herein.

The invention comprises a Fc-IL2v polypeptide complex as described herein for use in stimulating an immune response or function, such as T cell activity, in combination with (cells expressing) a recombinant MAB polypeptide and/or a recombinant MAB complex as described herein and optionally a targeting antibody (comprising G329 in the Fc domain according to EU numbering) as described herein, or alternatively for the manufacture of a medicament for use in stimulating an immune response or function, such as T cell activity, in combination with (cells expressing) a recombinant MAB polypeptide and/or a recombinant MAB complex as described herein and optionally a targeting antibody (comprising G329 in the Fc domain according to EU numbering) as described herein.

The invention comprises (cells expressing) a recombinant MAB polypeptide and/or a recombinant MAB complex as described herein for use in the treatment of cancer in combination with a Fc-IL2v polypeptide complex as described herein and optionally a targeting antibody (comprising G329 in the Fc domain according to EU numbering) as described herein, or alternatively for the manufacture of a medicament for the treatment of cancer in combination with a Fc-IL2v polypeptide complex as described herein and optionally a targeting antibody (comprising G329 in the Fc domain according to EU numbering) as described herein.

Some aspects of the invention comprise a targeting antibody (comprising G329 in the Fc domain according to EU numbering) as described herein for use in the treatment of cancer in combination with a Fc-IL2v polypeptide complex as described herein and (cells expressing) a recombinant MAB polypeptide and/or a recombinant MAB complex as described herein, or alternatively for the manufacture of a medicament for the treatment of cancer in combination with a Fc-IL2v polypeptide complex as described herein and (cells expressing) a recombinant MAB polypeptide and/or a recombinant MAB complex an as described herein.

In a preferred embodiment of the invention the recombinant Fc-IL2v polypeptide complex used in the above described combination treatments and medical uses of different diseases is a Fc-IL2v polypeptide complex characterized in comprising the polypeptide sequences of SEQ ID NO: 42 and SEQ ID NO: 44 or SEQ ID NO: 41 and SEQ ID NO:51, and the MAB polypeptide or MAB polypeptide complex used in such combination treatments is characterized in comprising the polypeptide sequences of SEQ ID NO: 63, SEQ ID NO: 67, SEQ ID NO: 71, SEQ ID NO: 146, SEQ ID NO:149, SEQ ID NO:151, SEQ ID NO: 154, SEQ ID NO:222, SEQ ID NO:235 and SEQ ID NO:255, or SEQ ID NO: 251 and SEQ ID NO:239.

In another aspect, the present invention provides a composition, e.g. a pharmaceutical composition, containing a Fc-IL2v polypeptide complex as described herein and (cells expressing) a recombinant MAB polypeptide and/or a recombinant MAB complex as described herein and optionally a targeting antibody (comprising G329 in the Fc domain according to EU numbering) as described herein formulated together with a pharmaceutically acceptable carrier.

As used herein. “pharmaceutically acceptable carrier” includes any and all solvents, dispersion media, coatings, antibacterial and antifungal agents, isotonic and absorption/resorption delaying agents, and the like that are physiologically compatible. Preferably, the carrier is suitable for injection or infusion.

A composition of the present invention can be administered by a variety of methods known in the art. As will be appreciated by the skilled artisan, the route and/or mode of administration will vary depending upon the desired results.

Pharmaceutically acceptable carriers include sterile aqueous solutions or dispersions and sterile powders for the preparation of sterile injectable solutions or dispersion. The use of such media and agents for pharmaceutically active substances is known in the art. In addition to water, the carrier can be, for example, an isotonic buffered saline solution.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient (effective amount). The selected dosage level will depend upon a variety of pharmacokinetic factors including the activity of the particular compositions of the present invention employed, or the ester, salt or amide thereof, the route of administration, the time of administration, the rate of excretion of the particular compound being employed, other drugs, compounds and/or materials used in combination with the particular compositions employed, the age, sex, weight, condition, general health and prior medical history of the patient being treated, and like factors well known in the medical arts.

In one aspect the invention provides a kit intended for the treatment of a disease, comprising in the same or in separate containers (a) a Fc-IL2v polypeptide complex as described herein, and (b) (cells expressing) a recombinant MAB polypeptide and/or a recombinant MAB complex as described herein and optionally (c) a targeting antibody (comprising G329 in the Fc domain according to EU numbering) as described herein, and optionally further comprising (d) a package insert comprising printed instructions directing the use of the combined treatment as a method for treating the disease. Moreover, the kit may comprise (a) a first container with a composition contained therein, wherein the composition comprises (cells expressing) a recombinant MAB polypeptide and/or a recombinant MAB complex as described herein: (b) a second container with a composition contained therein, wherein the composition comprises a Fc-IL2v polypeptide complex as described herein; and optionally (c) a third container with a composition contained therein, wherein the composition comprises a targeting antibody (comprising G329 in the Fc domain according to EU numbering) as described herein and optionally (d) fourth container with a composition contained therein, wherein the composition comprises a further cytotoxic or otherwise therapeutic agent. The kit in this embodiment of the invention may further comprise a package insert indicating that the compositions can be used to treat a particular condition. Alternatively, or additionally, the kit may further comprise a third (or fourth) container comprising a pharmaceutically-acceptable buffer, such as bacteriostatic water for injection (BWFI), phosphate-buffered saline. Ringer's solution and dextrose solution. It may further include other materials desirable from a commercial and user standpoint, including other buffers, diluents, filters, needles, and syringes.

In one aspect the invention provides a kit intended for the treatment of a disease, comprising (a) a container comprising a Fc-IL2v polypeptide complex as described herein, and (b) a package insert comprising instructions directing the use of the recombinant Fc-IL2v polypeptide complex in a combination therapy with (cells expressing) a recombinant MAB polypeptide and/or a recombinant MAB complex as described herein and optionally a targeting antibody (comprising G329 in the Fc domain according to EU numbering) as described herein as a method for treating the disease.

In another aspect the invention provides a kit intended for the treatment of a disease, comprising (a) a container comprising (cells expressing) a recombinant MAB polypeptide and/or a recombinant MAB complex as described herein, and (b) a package insert comprising instructions directing the use of the (cells expressing) the MAB polypeptide in a combination therapy with a Fc-IL2v polypeptide complex as described herein and optionally a targeting antibody (comprising G329 in the Fc domain according to EU numbering) as described herein as a method for treating the disease.

In another aspect the invention provides a kit intended for the treatment of a disease, comprising (a) a container comprising a targeting antibody (comprising G329 in the Fc domain according to EU numbering) as described herein, and (b) a package insert comprising instructions directing the use of the targeting antibody (comprising G329 in the Fc domain according to EU numbering) in a combination therapy with a Fc-IL2v polypeptide complex and (cells expressing) a recombinant MAB polypeptide and/or a recombinant MAB complex as described herein as a method for treating the disease.

In a further aspect the invention provides a medicament intended for the treatment of a disease, comprising a Fc-IL2v polypeptide complex as described herein, wherein said medicament is for use in a combination therapy with (cells expressing) a recombinant MAB polypeptide and/or a recombinant MAB complex as described herein and optionally a targeting antibody (comprising G329 in the Fc domain according to EU numbering) and optionally comprises a package insert comprising printed instructions directing the use of the combined treatment as a method for treating the disease.

The term “a method of treating” or its equivalent, when applied to, for example, cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed to induce an overall beneficial course of action.

The terms “administered in combination with” or “co-administration”, “co-administering”, “combination therapy” or “combination treatment” refer to the administration of the recombinant Fc-IL2v polypeptide complex as described herein and (cells expressing) the MAB polypeptide and optionally a targeting antibody (comprising G329 in the Fc domain according to EU numbering) as described herein e.g. as separate formulations/applications (or as one single formulation/application). The co-administration can be simultaneous or sequential in either order, wherein preferably there is a time period while both (or all) active agents simultaneously exert their biological activities. Said active agents are co-administered either simultaneously or sequentially (e.g. intravenous (i.v.)) through a continuous infusion. When both therapeutic agents are co-administered sequentially the dose is administered either on the same day in two separate administrations, or one of the agents is administered on day 1 and the second is co-administered on day 2 to day 7, preferably on day 2 to 4. Thus in one embodiment the term “sequentially” means within 7 days after the dose of the first component, preferably within 4 days after the dose of the first component; and the term “simultaneously” means at the same time. The term “co-administration” with respect to the maintenance doses of a Fc-IL2v polypeptide complex and/or (cells expressing) a MAB polypeptide and/or a targeting antibody (comprising G329 in the Fc domain according to EU numbering) means that the maintenance doses can be either co-administered simultaneously, if the treatment cycle is appropriate for all drugs, e.g. every week. Or the maintenance doses are co-administered sequentially, for example, doses of a Fc-IL2v polypeptide complex and (cells expressing) a MAB polypeptide and a targeting antibody (comprising G329 in the Fc domain according to EU numbering) are given on alternate weeks.

It is self-evident that the recombinant Fc-IL2v polypeptide complex. (cells expressing) the MAB polypeptide (complex) and/or the targeting antibody (comprising G329 in the Fc domain according to EU numbering) are administered to the patient in a “therapeutically effective amount” (or simply “effective amount”) which is the amount of the respective compound or combination that will elicit the biological or medical response of a tissue, system, animal or human that is being sought by the researcher, veterinarian, medical doctor or other clinician.

The amount of co-administration and the timing of co-administration will depend on the type (species, gender, age, weight, etc.) and condition of the patient being treated and the severity of the disease or condition being treated. Said Fc-IL2v polypeptide complex and/or (cells expressing) MAB polypeptide and/or a targeting antibody (comprising G329 in the Fc domain according to EU numbering) are suitably co-administered to the patient at one time or over a series of treatments e.g. on the same day or on the day after or at weekly intervals.

In addition to the recombinant Fc-IL2v polypeptide complex in combination with (cells expressing) a recombinant MAB polypeptide and/or a recombinant MAB complex and/or the targeting antibody (comprising G329 in the Fc domain according to EU numbering) as herein described, also a chemotherapeutic agent can be administered.

In one embodiment such additional chemotherapeutic agents, include, but are not limited to, anti-neoplastic agents including alkylating agents including: nitrogen mustards, such as mechlorethamine, cyclophosphamide, ifosfamide, melphalan and chlorambucil; nitrosourcas, such as carmustine (BCNU), lomustine (CCNU), and semustine (methyl-CCNU); Temodal™ (temozolamide), ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil (5FU), fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguamne, azathioprine, T-deoxycoformycin (pentostatin), erythrohydroxynony ladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actinomycin D, daunomycin (rubidomycin), doxorubicin, mitoxantrone, idarubicin, bleomycins, plicamycin (mithramycin), mitomycin C, and actinomycin: enzymes such as L-asparaginase; biological response modifiers such as interferon-alpha, IL-2, G-CSF and GM-CSF; miscellaneous agents including platinum coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o, p-DDD) and aminoglutethimide; hormones and antagonists including adrenocorticosteroid antagonists such as prednisone and equivalents, dexamethasone and aminoglutethimide; Gemzar™ (gemcitabine), progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Therapies targeting epigenetic mechanism including, but not limited to, histone deacetylase inhibitors, demethylating agents (e.g., Vidaza) and release of transcriptional repression (ATRA) therapies can also be combined with the antigen binding proteins. In one embodiment the chemotherapeutic agent is selected from the group consisting of taxanes (like e.g. paclitaxel (Taxol), docetaxel (Taxotere), modified paclitaxel (e.g., Abraxane and Opaxio), doxorubicin, sunitinib (Sutent), sorafenib (Nexavar), and other multikinase inhibitors, oxaliplatin, cisplatin and carboplatin, etoposide, gemcitabine, and vinblastine. In one embodiment the chemotherapeutic agent is selected from the group consisting of taxanes (like e.g. taxol (paclitaxel), docetaxel (Taxotere), modified paclitaxel (e.g. Abraxane and Opaxio). In one embodiment, the additional chemotherapeutic agent is selected from 5-fluorouracil (5-FU), leucovorin, irinotecan, or oxaliplatin. In one embodiment the chemotherapeutic agent is 5-fluorouracil, leucovorin and irinotecan (FOLFIRI). In one embodiment the chemotherapeutic agent is 5-fluorouracil, and oxaliplatin (FOLFOX).

Specific examples of combination therapies with additional chemotherapeutic agents include, for instance, therapies taxanes (e.g., docetaxel or paclitaxel) or a modified paclitaxel (e.g., Abraxane or Opaxio), doxorubicin), capecitabine and/or bevacizumab (Avastin) for the treatment of breast cancer; therapies with carboplatin, oxaliplatin, cisplatin, paclitaxel, doxorubicin (or modified doxorubicin (Caclyx or Doxil)), or topotecan (Hycamtin) for ovarian cancer, the therapies with a multi-kinase inhibitor, MKI, (Sutent, Nexavar, or 706) and/or doxorubicin for treatment of kidney cancer; therapies with oxaliplatin, cisplatin and/or radiation for the treatment of squamous cell carcinoma; therapies with taxol and/or carboplatin for the treatment of lung cancer.

Therefore, in one embodiment the additional chemotherapeutic agent is selected from the group of taxanes (docetaxel or paclitaxel or a modified paclitaxel (Abraxane or Opaxio), doxorubicin, capecitabine and/or bevacizumab for the treatment of breast cancer.

In one embodiment, the combination therapy of Fc-IL2v with (cells expressing) a recombinant MAB polypeptide and/or a recombinant MAB complex and/or the targeting antibody (comprising G329 in the Fc domain according to EU numbering) is one in which no chemotherapeutic agents are administered.

The invention comprises also a method for the treatment of a patient suffering from such disease as described herein.

The invention further provides a method for the manufacture of a pharmaceutical composition comprising an effective amount of a Fc-IL2v polypeptide complex according to the invention as described herein and (cells expressing) a recombinant MAB polypeptide and/or a recombinant MAB complex according to the invention as described herein and optionally the a targeting antibody (comprising G329 in the Fc domain according to EU numbering) according to the invention as described herein together with a pharmaceutically acceptable carrier and the use of the recombinant Fc-IL2v polypeptide complex and (cells expressing) a recombinant MAB polypeptide and/or a recombinant MAB complex according to the invention as described herein and optionally a targeting antibody (comprising G329 in the Fc domain according to EU numbering) according to the invention as described herein for such a method.

The invention further provides the use of a Fc-IL2v polypeptide complex according to the invention as described herein and (cells expressing) a recombinant MAB polypeptide and/or a recombinant MAB complex according to the invention as described herein and optionally a targeting antibody (comprising G329 in the Fc domain according to EU numbering) according to the invention as described herein in an effective amount for the manufacture of a pharmaceutical agent, preferably together with a pharmaceutically acceptable carrier, for the treatment of a patient suffering from cancer.

Each of the components of the combination treatment is explained in more detail herein below.

### Recombinant Fc Domain-IL2 Variant (Fc-IL2v) Polypeptide Complex

### Fc Domains Polypeptides

The recombinant Fc-IL2v polypeptide complex of the present invention comprises a variant Fc domain as described further below. The antigen-binding moiety of the MAB polypeptide (complex) of the present disclosure provides for binding to the variant Fc domain. Variant Fc domains according to the present disclosure comprise an amino acid sequence comprising at least one amino acid difference relative to a reference Fc domain to which the MAB polypeptide (complex) does not bind.

As used herein, an “Fc domain” refers to a polypeptide complex formed by interaction between two polypeptides, each polypeptide comprising the CH2-CH3 region of an immunoglobulin (Ig) heavy chain constant sequence.

Immunoglobulins of type G (i.e. IgG) are ˜150 kDa glycoproteins comprising two heavy chains and two light chains. From N- to C-terminus, the heavy chains comprise a VH followed by a heavy chain constant region comprising three constant domains (CH1. CH2, and CH3), and similarly the light chains comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

Herein, a “CH2 domain” refers to an amino acid sequence corresponding to the CH2 domain of an immunoglobulin (Ig). The CH2 domain is the region of an Ig formed by positions 231 to 340 of the immunoglobulin constant domain, according to the EU numbering system described in Edelman et al., Proc Natl Acad Sci USA (1969) 63 (1): 78-85. A “CH3 domain” refers to an amino acid sequence corresponding to the CH3 domain of an immunoglobulin (Ig). The CH3 domain is the region of an Ig formed by positions 341 to 447 of the immunoglobulin constant domain, according to the EU numbering system described in Edelman et al., Proc Natl Acad Sci USA (1969) 63 (1): 78-85. A “CH2-CH3 region” refers to an amino acid sequence corresponding to the CH2 and CH3 domains of an immunoglobulin (Ig). The CH2-CH3 region is the region of an Ig formed by positions 231 to 447 of the immunoglobulin constant domain, according to the EU numbering system described in Edelman et al., Proc Natl Acad Sci USA (1969) 63 (1): 78-85.

In some embodiments, a CH2 domain, CH3 domain and/or a CH2-CH3 region according to the present disclosure corresponds to the CH2 domain/CH3 domain/CH2-CH3 region of an IgG (e.g. IgG1, IgG2, IgG3, IgG4), IgA (e.g. IgA1, IgA2), IgD, IgE or IgM. In some embodiments, the CH2 domain, CH3 domain and/or a CH2-CH3 region corresponds to the CH2 domain/CH3 domain/CH2-CH3 region of a human IgG (e.g. hIgG1, hIgG2, hIgG3, hIgG4), hIgA (e.g. hIgA1, hIgA2), hIgD, hIgE or hIgM. In some embodiments, the CH2 domain, CH3 domain and/or a CH2-CH3 region corresponds to the CH2 domain/CH3 domain/CH2-CH3 region of a human IgG1 allotype (e.g. Glm1, Glm2, Glm3 or Glm17).

It will be appreciated that an Fc domain according to the present disclosure may form part of a larger molecule comprising the Fc domain. For example, a variant Fc domain according to the present disclosure may be comprised in a recombinant Fc domain-IL2 variant (Fc-IL2v) polypeptide complex as further described below. In some aspect, the recombinant Fc-IL2v polypeptide complex may be further comprised in an antigen-binding molecule (e.g. an antibody) comprising an antigen-binding moiety specific for a target antigen, a variant Fc domain, and an IL2 variant according to the present disclosure.

Fc domains provide for interaction with Fc receptors and other molecules of the immune system to bring about functional effects. Fc-mediated effector functions are reviewed e.g. in Jefferis et al., Immunol Rev 1998 163:59-76 (hereby incorporated by reference in its entirety), and are brought about through Fc-mediated recruitment and activation of immune cells (e.g. macrophages, dendritic cells, neutrophils, basophils, eosinophils, platelets, mast cells. NK cells and T cells) through interaction between the Fc region and Fc receptors expressed by the immune cells, recruitment of complement pathway components through binding of the Fc region to complement protein C1q, and consequent activation of the complement cascade. Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

The CH2-CH3 region sequence of the human IgG1 Glm1 allotype is shown in SEQ ID NO:1. The CH2-CH3 region sequence of the human IgG1 Glm3 allotype is shown in SEQ ID NO:2. The CH2-CH3 region sequence of human IgG2 is shown in SEQ ID NO:3. The CH2-CH3 region sequence of human IgG3 is shown in SEQ ID NO:4. The CH2-CH3 region sequence of human IgG4 is shown in SEQ ID NO: 5.

Variant Fc domains according to the present disclosure comprise an amino acid sequence comprising at least one amino acid difference relative to a reference Fc domain. For example, a “variant CH2-CH3 region” according to the present disclosure comprises an amino acid sequence comprising at least one amino acid difference relative to a reference CH2-CH3 domain. The CH2-CH3 region sequence of the human IgG1 Glm1 allotype is shown in SEQ ID NO:1. The CH2-CH3 region sequence of the human IgG1 Glm3 allotype is shown in SEQ ID NO:2. The CH2-CH3 region sequence of human IgG2 is shown in SEQ ID NO:3. The CH2-CH3 region sequence of human IgG3 is shown in SEQ ID NO:4. The CH2-CH3 region sequence of human IgG4 is shown in SEQ ID NO:5. In some embodiments, the reference CH2-CH3 domain comprises a sequence selected from the group consisting of SEQ ID NO:1, SEQ ID NO: 2, SEQ ID NO:3, SEQ ID NO:4, and SEQ ID NO:5. In some embodiments, the reference CH2-CH3 domain comprises a sequence selected from the group consisting of SEQ ID NO: 1 and SEQ ID NO:2. In a preferred embodiment, the reference CH2-CH3 domain comprises the sequence of SEQ ID NO: 1.

In some embodiments, a reference Fc domain according to the present disclosure comprises two polypeptides, wherein each polypeptide comprises a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO: 1. In some embodiments, a reference Fc domain comprises two polypeptides, wherein each polypeptide comprises a CH2-CH3 region comprising or consisting of SEQ ID NO: 1.

In some embodiments, a reference Fc domain comprises two polypeptides, wherein each polypeptide comprises a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:2. In some embodiments, a reference Fc domain comprises two polypeptides, wherein each polypeptide comprises a CH2-CH3 region comprising or consisting of SEQ ID NO:2.

In some embodiments, a reference Fc domain comprises two polypeptides, wherein each polypeptide comprises a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:3. In some embodiments, a reference Fc domain comprises two polypeptides, wherein each polypeptide comprises a CH2-CH3 region comprising or consisting of SEQ ID NO:3.

In some embodiments, a reference Fc domain comprises two polypeptides, wherein each polypeptide comprises a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:4. In some embodiments, a reference Fc domain comprises two polypeptides, wherein each polypeptide comprises a CH2-CH3 region comprising or consisting of SEQ ID NO:4.

In some embodiments, a reference Fc domain comprises two polypeptides, wherein each polypeptide comprises a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:5. In some embodiments, a reference Fc domain comprises two polypeptides, wherein each polypeptide comprises a CH2-CH3 region comprising or consisting of SEQ ID NO:5.

A variant Fc domain according to the present disclosure may comprise an amino acid difference relative to the amino acid sequence of one or both of the polypeptides of a reference Fc domain according to the present disclosure.

In some embodiments, a variant Fc domain according to the present disclosure comprises two polypeptides, each polypeptide comprising a CH2-CH3 region, and wherein one or both of the CH2-CH3 regions comprise an amino acid sequence which is non-identical to SEQ ID NO: 1. In some embodiments, a variant Fc domain according to the present disclosure comprises two polypeptides, each polypeptide comprising a CH2-CH3 region, and wherein one or both of the CH2-CH3 regions comprise an amino acid sequence having one or more (e.g. 1, 2, 3, 4, 5 or more) amino acid differences relative to SEQ ID NO:1. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:1, wherein one or both of the CH2-CH3 regions comprises an amino acid sequence which is non-identical to SEQ ID NO:1. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:1, wherein one or both of the CH2-CH3 regions comprise an amino acid sequence having one or more (e.g. 1, 2, 3, 4, 5 or more) amino acid differences relative to SEQ ID NO:1.

In some embodiments, a variant Fc domain according to the present disclosure comprises two polypeptides, each polypeptide comprising a CH2-CH3 region, and wherein one or both of the CH2-CH3 regions comprise an amino acid sequence which is non-identical to SEQ ID NO:2. In some embodiments, a variant Fc domain according to the present disclosure comprises two polypeptides, each polypeptide comprising a CH2-CH3 region, and wherein one or both of the CH2-CH3 regions comprise an amino acid sequence having one or more (e.g. 1, 2, 3, 4, 5 or more) amino acid differences relative to SEQ ID NO:2. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO: 2, wherein one or both of the CH2-CH3 regions comprises an amino acid sequence which is non-identical to SEQ ID NO:2. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:2, wherein one or both of the CH2-CH3 regions comprise an amino acid sequence having one or more (e.g. 1, 2, 3, 4, 5 or more) amino acid differences relative to SEQ ID NO:2.

In some embodiments, a variant Fc domain according to the present disclosure comprises two polypeptides, each polypeptide comprising a CH2-CH3 region, and wherein one or both of the CH2-CH3 regions comprise an amino acid sequence which is non-identical to SEQ ID NO:3. In some embodiments, a variant Fc domain according to the present disclosure comprises two polypeptides, each polypeptide comprising a CH2-CH3 region, and wherein one or both of the CH2-CH3 regions comprise an amino acid sequence having one or more (e.g. 1, 2, 3, 4, 5 or more) amino acid differences relative to SEQ ID NO:3. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO: 3, wherein one or both of the CH2-CH3 regions comprises an amino acid sequence which is non-identical to SEQ ID NO:3. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:3, wherein one or both of the CH2-CH3 regions comprise an amino acid sequence having one or more (e.g. 1, 2, 3, 4, 5 or more) amino acid differences relative to SEQ ID NO:3.

In some embodiments, a variant Fc domain according to the present disclosure comprises two polypeptides, each polypeptide comprising a CH2-CH3 region, and wherein one or both of the CH2-CH3 regions comprise an amino acid sequence which is non-identical to SEQ ID NO:4. In some embodiments, a variant Fc domain according to the present disclosure comprises two polypeptides, each polypeptide comprising a CH2-CH3 region, and wherein one or both of the CH2-CH3 regions comprise an amino acid sequence having one or more (e.g. 1, 2, 3, 4, 5 or more) amino acid differences relative to SEQ ID NO:4. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO: 4, wherein one or both of the CH2-CH3 regions comprises an amino acid sequence which is non-identical to SEQ ID NO:4. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:4, wherein one or both of the CH2-CH3 regions comprise an amino acid sequence having one or more (e.g. 1, 2, 3, 4, 5 or more) amino acid differences relative to SEQ ID NO:4.

In some embodiments, a variant Fc domain according to the present disclosure comprises two polypeptides, each polypeptide comprising a CH2-CH3 region, and wherein one or both of the CH2-CH3 regions comprise an amino acid sequence which is non-identical to SEQ ID NO:5. In some embodiments, a variant Fc domain according to the present disclosure comprises two polypeptides, each polypeptide comprising a CH2-CH3 region, and wherein one or both of the CH2-CH3 regions comprise an amino acid sequence having one or more (e.g. 1, 2, 3, 4, 5 or more) amino acid differences relative to SEQ ID NO:5. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO: 5, wherein one or both of the CH2-CH3 regions comprises an amino acid sequence which is non-identical to SEQ ID NO:5. In some embodiments, each CH2-CH3 region of the variant Fc domain comprises or consists of an amino acid sequence having at least 70% sequence identity, more preferably one of at least 275%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:5, wherein one or both of the CH2-CH3 regions comprise an amino acid sequence having one or more (e.g. 1, 2, 3, 4, 5 or more) amino acid differences relative to SEQ ID NO:5.

In some embodiments, each CH2-CH3 region of a variant Fc domain according to the present disclosure comprises an amino acid difference relative to a reference Fc domain according to the present disclosure. In some embodiments, the amino acid sequences of the CH2-CH3 regions of the constituent polypeptides of a variant Fc domain according to the present disclosure are identical (i.e. they have the same amino acid sequence).

The amino acid difference of a variant Fc domain according to the present disclosure (relative to a reference Fc domain) may influence an Fc-mediated function.

Modifications to Fc domains that influence Fc-mediated function are known in the art, such as those described e.g. in Wang et al., Protein Cell (2018) 9 (1): 63-73 and Saunders et al., Front Immunol. (2019) 10:1296, both of which are hereby incorporated by reference in their entirety. Exemplary Fc domain modifications known to influence Fc-mediated function are summarised in Table 1 of Wang et al., Protein Cell (2018) 9 (1): 63-73, and in Tables 1, 2 and 3 of Saunders et al., Front Immunol. (2019) 10:1296. In some embodiments, the variant Fc domain of the present disclosure comprises an Fc domain comprising an amino acid difference relative to a reference Fc domain (e.g. a reference Fc domain according to the present disclosure) that increases or reduces an Fc-mediated function.

In some embodiments, the variant Fc domain comprises an amino acid difference relative to a reference Fc domain that increases an Fc-mediated function. In some embodiments, the variant Fc domain comprises an amino acid difference relative to a reference Fc domain that increases ADCC. ADCP and/or CDC. Accordingly, in some embodiments the variant Fc domain displays an increased level of an Fc-mediated function as compared to the reference Fc domain. In some embodiments, the variant Fc domain displays increased ADCC. ADCP and/or CDC as compared to the reference Fc domain.

In some embodiments, the variant Fc domain comprises an amino acid difference relative to a reference Fc domain that increases binding to an Fc receptor (e.g. a Fcγ receptor, e.g. FcγRI, FcγRIIa, FcγRIIb, FcγRIIc, FcγRIIIa and/or FcγRIIIb). In some embodiments, the variant Fc domain comprises an amino acid difference relative to a reference Fc domain that increases binding to FcRn. In some embodiments, the variant Fc domain comprises an amino acid difference relative to a reference Fc domain that increases binding to a complement protein (e.g. C1q). In some embodiments, the variant Fc domain comprises an amino acid difference relative to a reference Fc domain to increase hexamerisation of an antigen-binding molecule comprising the variant Fc domain. In some embodiments, the Fc domain comprises an amino acid difference relative to a reference Fc domain that increases the half-life of an antigen-binding molecule comprising the variant Fc domain. Accordingly, in some embodiments the variant Fc domain displays increased binding to an Fc receptor (e.g. a Fcγ receptor, e.g. FcγRI, FcγRIIa, FcγRIIb, FcγRIIc, FcγRIIIa and/or FcγRIIIb) as compared to the reference Fc domain. In some embodiments the variant Fc domain displays increased binding to FcRn as compared to the reference Fc domain. In some embodiments the variant Fc domain displays increased binding to a complement protein (e.g. C1q) as compared to the reference Fc domain. In some embodiments an antigen-binding molecule comprising the variant Fc domain displays increased hexamerisation as compared to an antigen-binding molecule comprising the reference Fc domain. In some embodiments an antigen-binding molecule comprising the variant Fc domain displays an increased half-life as compared to an antigen-binding molecule comprising the reference Fc domain.

In some embodiments, the variant Fc domain comprises an amino acid difference relative to a reference Fc domain that reduces an Fc-mediated function. In some embodiments, the variant Fc domain comprises an amino acid difference relative to a reference Fc domain that reduces ADCC, ADCP and/or CDC. Accordingly, in some embodiments the variant Fc domain displays a reduced level of an Fc-mediated function as compared to the reference Fc domain. In some embodiments, the variant Fc domain displays reduced ADCC. ADCP and/or CDC as compared to the reference Fc domain.

In some embodiments, the variant Fc domain comprises an amino acid difference relative to a reference Fc domain that reduces binding to an Fc receptor (e.g. a Fcγ receptor, e.g. FcγRI, FcγRIIa, FcγRIIb, FcγRIIc, FcγRIIIa and/or FcγRIIIb). In some embodiments, the variant Fc domain comprises an amino acid difference relative to a reference Fc domain that reduces binding to FcRn. In some embodiments, the variant Fc domain comprises an amino acid difference relative to a reference Fc domain that reduces binding to a complement protein (e.g. C1q). In some embodiments, the variant Fc domain comprises an amino acid difference relative to a reference Fc domain to increase hexamerisation of an antigen-binding molecule comprising the variant Fc domain. In some embodiments, the Fc domain comprises an amino acid difference relative to a reference Fc domain that reduces the half-life of an antigen-binding molecule comprising the variant Fc domain. Accordingly, in some embodiments the variant Fc domain displays reduced binding to an Fc receptor (e.g. a Fcγ receptor, e.g. FcγRI, FcγRIIa, FcγRIIb, FcγRIIc, FcγRIIIa and/or FcγRIIIb) as compared to the reference Fc domain. In some embodiments the variant Fc domain displays reduced binding to FcRn as compared to the reference Fc domain. In some embodiments the variant Fc domain displays reduced binding to a complement protein (e.g. C1q) as compared to the reference Fc domain. In some embodiments an antigen-binding molecule comprising the variant Fc domain displays reduced hexamerisation as compared to an antigen-binding molecule comprising the reference Fc domain. In some embodiments an antigen-binding molecule comprising the variant Fc domain displays a reduced half-life as compared to an antigen-binding molecule comprising the reference Fc domain.

In some embodiments, the variant Fc domain comprises a CH2-CH3 region comprising an amino acid difference at position 329, relative to the amino acid sequence of a CH2-CH3 region of the reference Fc domain. In some embodiments, the variant Fc domain comprises a CH2-CH3 region comprising an amino acid difference at positions 234, 235 and 329, relative to the amino acid sequence of a CH2-CH3 region of the reference Fc domain.

In some embodiments, the variant Fc domain comprises a CH2-CH3 region comprising an amino acid difference at P329, relative to the amino acid sequence of a CH2-CH3 region of the reference Fc domain. In some embodiments, the variant Fc domain comprises a CH2-CH3 region comprising an amino acid difference at positions L234. L235 and P329, relative to the amino acid sequence of a CH2-CH3 region of the reference Fc domain.

In some embodiments, the variant Fc domain comprises a CH2-CH3 region comprising the amino acid substitution P329G, relative to the amino acid sequence of a CH2-CH3 region of the reference Fc domain. In some embodiments, the variant Fc domain comprises a CH2-CH3 region comprising the amino acid substitutions L234A. L235A and P329G relative to the amino acid sequence of a CH2-CH3 region of the reference Fc domain.

In some embodiments, a variant Fc domain according to the present disclosure comprises a polypeptide comprising a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:6 or 8, wherein the CH2-CH3 region comprises G329. In some embodiments, a variant Fc domain according to the present disclosure comprises two polypeptides, each polypeptide comprising a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:6 or 8, wherein the CH2-CH3 region comprises G329. In some embodiments, a variant Fc domain according to the present disclosure comprises one or more (e.g. two) polypeptides comprising the amino acid sequence of SEQ ID NO:6 or 8.

### IL-2 Pathway and IL2 Variant Polypeptides

In one aspect of the present invention, the variant Fc domain (or variant CH2-CH3 region) as described hereinabove is fused to an IL2 variant as described hereinbelow to form a Fc domain-IL2 variant (Fc-IL2v) polypeptide complex.

The ability of IL-2 to expand and activate lymphocyte and NK cell populations both in vitro and in vivo explains the anti-tumor effects of IL-2. However, as a regulatory mechanism to prevent excessive immune responses and potential autoimmunity. IL-2 leads to activation-induced cell death (AICD) and renders activated T-cells susceptible to Fas-mediated apoptosis.

Moreover, IL-2 is involved in the maintenance and expansion of peripheral CD4⁺ CD25⁺ T_{reg} cells (Fontenot J D, Rasmussen J P, Gavin M A, et al. A function for interleukin 2 in Foxp3 expressing regulatory T cells. Nat Immunol. 2005; 6:1142-1151; D'Cruz L M, Klein L. Development and function of agonist-induced CD25⁺ Foxp3⁺ regulatory T cells in the absence of interleukin 2 signaling. Nat Immunol. 2005; 6:1152 1159; Maloy K J, Powrie F. Fueling regulation: IL-2 keeps CD4⁺ T_{reg} cells fit. Nat Immunol. 2005; 6:1071-1072). These cells suppress effector T-cells from destroying self or target, either through cell-cell contact or through release of immunosuppressive cytokines, such as IL-10 or transforming growth factor (TGF)-β. Depletion of T_{reg} cells was shown to enhance IL-2-induced anti-tumor immunity (Imai H, Saio M, Nonaka K, et al. Depletion of CD4+CD25+ regulatory T cells enhances interleukin-2-induced antitumor immunity in a mouse model of colon adenocarcinoma. Cancer Sci. 2007:98:416-423).

IL-2 also plays a significant role in memory CD8+ T-cell differentiation during primary and secondary expansion of CD8+ T cells. IL-2 seems to be responsible for optimal expansion and generation of effector functions following primary antigenic challenge. During the contraction phase of an immune response where most antigen-specific CD8+ T cells disappear by apoptosis. IL-2 signals are able to rescue CD8+ T cells from cell death and provide a durable increase in memory CD8+ T-cells. At the memory stage, CD8+ T-cell frequencies can be boosted by administration of exogenous IL-2. Moreover, only CD8+ T cells that have received IL-2 signals during initial priming are able to mediate efficient secondary expansion following renewed antigenic challenge. Thus. IL-2 signals during different phases of an immune response are key in optimizing CD8+ T-cell functions, thereby affecting both primary and secondary responses of these T cells (Adv Exp Med Biol. 2010; 684:28-41. The role of interleukin-2 in memory CD8 cell differentiation. Boyman O l, Cho J H, Sprent J).

Based on its anti-tumor efficacy, high-dose IL-2 (aldesleukin, marketed as Proleukin®) treatment has been approved for use in patients with metastatic renal cell carcinoma (RCC) and malignant melanoma in the US, and for patients with metastatic RCC in the European Union. However, as a consequence of the mode of action of IL-2, the systemic and untargeted application of IL-2 may considerably compromise anti-tumor immunity via induction of T_{reg} cells and AICD. An additional concern of systemic IL-2 treatment is related to severe side-effects upon intravenous administration, which include severe cardiovascular, pulmonary edema, hepatic, gastrointestinal (GI), neurological, and hematological events (Proleukin (aldesleukin) Summary of Product Characteristics [SmPC]: http://www.medicines.org.uk/emc/medicine/19322/SPC/(accessed May 27, 2013)). Low-dose IL-2 regimens have been tested in patients, although at the expense of suboptimal therapeutic results. Taken together, therapeutic approaches utilizing IL-2 may be useful for cancer therapy if the liabilities associated with its application can be overcome.

In particular, mutant IL-2 (e.g., a quadruple mutant known as IL-2 qm) has been designed to overcome the limitations of wildtype IL-2 (e.g., aldesleukin) or first generation IL-2-based immunoconjugates by eliminating the binding to the IL-2Rα subunit (CD25). This mutant IL-2 qm has been coupled to various tumor-targeting antibodies such as a humanized antibody directed against CEA and an antibody directed against FAP, described in WO 2012/146628 and WO 2012/107417. In addition, the Fc region of the antibody has been modified to prevent binding to Fey receptors and the C1q complex. The resulting tumor-targeted IL-2 variant immunoconjugates (e.g., CEA-targeted IL-2 variant immunoconjugate and FAP-targeted IL-2 variant immunoconjugate) have been shown in nonclinical in vitro and in vivo experiments to be able to eliminate tumor cells.

The term “IL-2” or “human IL-2” refers to the human IL-2 protein including wildtype and variants comprising one or more mutations in the amino acid sequence of wildtype IL-2, for example as shown in SEQ ID NO: 38 having a C125A substitution to avoid the formation of disulphide-bridged IL-2 dimers. IL-2 may also be mutated to remove N- and/or O-glycosylation sites.

Variant or mutant IL-2 polypeptides (“IL-2 variant polypeptide” or “IL2v polypeptide”) according to the present disclosure comprise an amino acid sequence comprising at least one amino acid difference relative to a reference IL-2 polypeptide. For example, in a preferred embodiment, the IL-2 variant polypeptide according to the present disclosure comprises an amino acid sequence comprising at least one amino acid difference relative to human IL-2 (shown as SEQ ID NO: 40).

As described in WO 2012/146628, an IL-2 mutant has reduced binding affinity to the α-subunit of the IL-2 receptor. Together with the β- and γ-subunits (also known as CD122 and CD132, respectively), the α-subunit (also known as CD25) forms the heterotrimeric high affinity IL-2 receptor, while the dimeric receptor consisting only of the β- and γ-subunits is termed the intermediate-affinity IL-2 receptor. As described in WO 2012/146628, an IL-2 mutant polypeptide with reduced binding to the α-subunit of the IL-2 receptor has a reduced ability to induce IL-2 signalling in regulatory T cells, induces less activation-induced cell death (AICD) in T cells, and has a reduced toxicity profile in vivo, compared to a wild-type IL-2 polypeptide. The use of such an IL-2 mutant with reduced toxicity is particularly advantageous in Fc-IL2v polypeptide complexes, having a long serum half-life due to the presence of an Fc domain. The IL-2 mutant may comprise at least one amino acid mutation that reduces or abolishes the affinity of the IL-2 mutant to the α-subunit of the IL-2 receptor (CD25) but preserves the affinity of the IL-2 mutant to the intermediate-affinity IL-2 receptor (consisting of the β- and γ-subunits of the IL-2 receptor), compared to wildtype IL-2. The one or more amino acid mutations may be amino acid substitutions. The IL-2 mutant may comprise one, two or three amino acid substitutions at one, two or three position(s) selected from the positions corresponding to residue 42, 45, and 72 of human IL-2 (shown as SEQ ID NO: 40). The IL-2 mutant may comprise three amino acid substitutions at the positions corresponding to residue 42, 45 and 72 of human IL-2. The IL-2 mutant may be a mutant of human IL-2. The IL-2 mutant may be human IL-2 comprising the amino acid substitutions F42A, Y45A and L72G. The IL-2 mutant may additionally comprise an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular IL-2 mutant useful in the invention comprises four amino acid substitutions at positions corresponding to residues 3, 42, 45 and 72 of human IL-2 (shown as SEQ ID NO: 40). Specific amino acid substitutions are T3A, F42A, Y45A and L72G. As demonstrated in the Examples of WO 2012/146628, said quadruple mutant IL-2 polypeptide (IL-2 qm) exhibits no detectable binding to CD25, reduced ability to induce apoptosis in T cells, reduced ability to induce IL-2 signaling in T_{reg} cells, and a reduced toxicity profile in vivo. However, it retains ability to activate IL-2 signaling in effector cells, to induce proliferation of effector cells, and to generate IFN-γ as a secondary cytokine by NK cells. The IL-2 mutant according to any of the above descriptions may comprise additional mutations that provide further advantages such as increased expression or stability. For example, the cysteine at position 125 may be replaced with a neutral amino acid such as alanine, to avoid the formation of disulfide-bridged IL-2 dimers. Thus, the IL-2 mutant may comprise an additional amino acid mutation at a position corresponding to residue 125 of human IL-2. Said additional amino acid mutation may be the amino acid substitution C125A. The IL-2 mutant may comprise the polypeptide sequence of SEQ ID NO: 38. The IL-2 mutant may comprise in addition an amino acid substitution at position corresponding to 126 of human IL-2 (shown in SEQ ID NO:40), specifically the amino acid substitution Q126T. The Q126T substitution further reduces binding to CD25, leading to further reduced ability to induce apoptosis in T cells, reduced ability to induce IL-2 signaling in T_{reg} cells, and a reduced toxicity profile in vivo. This further IL-2 mutant may comprise the polypeptide sequence of SEQ ID NO: 39.

The recombinant Fc-IL2v polypeptide complex used in the combination therapy described herein comprises a variant Fc domain as hereinbefore described, and an IL-2 mutant, particularly a mutant of human IL-2, having reduced binding affinity to the α-subunit of the IL-2 receptor (as compared to wild-type IL-2, e.g. human IL-2 shown as SEQ ID NO: 40), such as an IL-2 comprising: i) one, two or three amino acid substitution(s) at one, two or three position(s) selected from the positions corresponding to residues 42, 45 and 72 of human IL-2 shown as SEQ ID NO: 40, for example three substitutions at three positions, for example the specific amino acid substitutions F42A, Y45A and L72G; or ii) the features as set out in i) plus an amino acid substitution at a position corresponding to residue 3 of human IL-2 shown as SEQ ID NO: 40, for example the specific amino acid substitution T3A; or iii) four amino acid substitutions at positions corresponding to residues 3, 42, 45 and 72 of human IL-2 shown as SEQ ID NO: 40, for example the specific amino acid substitutions T3A, F42A, Y45A and L72G, or iv) five amino acid substitutions at positions corresponding to residues 3, 42, 45, 72 and 126 of human IL-2 shown as SEQ ID NO: 40, for example the specific amino acid substitutions T3A, F42A, Y45A, L72G, and Q126T.

In some aspects of the present invention, the recombinant Fc-IL2v polypeptide complex does not comprise an antigen binding moiety. In the appended Examples, it has been shown that an Fc-IL2v polypeptide complex consisting of one polypeptide consisting of a CH2-CH3 region comprising P329 according to EU numbering fused to an IL-2 mutant, and one polypeptide consisting of a CH2-CH3 region comprising P329 according to EU numbering was capable of stronger activation of T cell compared to a Fc-IL2v polypeptide complex further comprising an antigen binding moiety. Without being bound to theory. Fc-IL2v polypeptide complexes not comprising an antigen binding moiety might have sterical advantages compared to more complex molecules. Furthermore, it might be desirable to target the recombinant Fc-IL2v polypeptide complexes to cells expressing the MAB polypeptide according to the present invention only by means of the interaction between MAB antigen binding moiety and CH2-CH3 region comprising P329 according to EU numbering.

In some aspects, the recombinant Fc-IL2v polypeptide complex does not comprise a variable fragment (Fv) moiety, a single-chain Fv (scFv) moiety, a fragment antigen-binding (Fab) moiety, a single-chain Fab moiety (scFab), a crossFab moiety, a Fab moiety, a Fab-SH moiety, a F(ab′)₂ moiety, a diabody moiety, a triabody moiety, an scFv-Fc moiety, a minibody moiety, a heavy chain only antibody (HCAb) moiety, or a single domain antibody (dAb, VHH) moiety.

In some aspects, the recombinant Fc-IL2v polypeptide complex does not comprise Fab or crossFab antigen binding moiety.

In some embodiments, the recombinant Fc-IL2v polypeptide complex according to the present disclosure comprises:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
(ii) a second polypeptide comprising a variant CH2-CH3 region comprising G329 according to EU numbering,
   wherein the recombinant Fc-IL2v polypeptide complex does not comprise an antigen binding moiety.

In some embodiments, the recombinant Fc-IL2v polypeptide complex according to the present disclosure comprises:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40, wherein the first polypeptide comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to an amino acid sequence selected from the group consisting of SEQ ID NO:43, SEQ ID NO:44, SEQ ID NO:47, and SEQ ID NO: 48; and
(ii) a second polypeptide comprising a variant CH2-CH3 region comprising G329 according to EU numbering, wherein the second polypeptide comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:42.
   wherein the recombinant Fc-IL2v polypeptide complex does not comprise an antigen binding moiety, wherein the first and second polypeptide are capable of stable association.

In some embodiments, the recombinant Fc-IL2v polypeptide complex according to the present disclosure comprises:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40, wherein the first polypeptide comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to an amino acid sequence selected from the group consisting of SEQ ID NO:50, SEQ ID NO:51, SEQ ID NO: 52 and SEQ ID NO:53 and
(ii) a second polypeptide comprising a variant CH2-CH3 region comprising G329 according to EU numbering, wherein the second polypeptide comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:41,
   wherein the recombinant Fc-IL2v polypeptide complex does not comprise an antigen binding moiety, wherein the first and second polypeptide are capable of stable association.

In some embodiments, the recombinant Fc-IL2v polypeptide complex according to the present disclosure comprises:
(i) a first polypeptide comprising an amino acid sequence selected from the group consisting of SEQ ID NO:43, SEQ ID NO:44, SEQ ID NO:47, and SEQ ID NO:48; and
(ii) a second polypeptide comprising the amino acid sequence of SEQ ID NO:42.
   wherein the recombinant Fc-IL2v polypeptide complex does not comprise an antigen binding moiety.

In some embodiments, the recombinant Fc-IL2v polypeptide complex according to the present disclosure comprises:
(i) a first polypeptide comprising an amino acid sequence selected from the group consisting of SEQ ID NO:50, SEQ ID NO:51, SEQ ID NO:52 and SEQ ID NO:53; and
(ii) a second polypeptide comprising the amino acid sequence of SEQ ID NO:41.
   wherein the recombinant Fc-IL2v polypeptide complex does not comprise an antigen binding moiety.

In some embodiments, the recombinant Fc-IL2v polypeptide complex according to the present disclosure comprises:
(i) a first polypeptide consisting of an amino acid sequence selected from the group consisting of SEQ ID NO:43, SEQ ID NO:44, SEQ ID NO:47, and SEQ ID NO:48; and
(ii) a second polypeptide consisting of the amino acid sequence of SEQ ID NO:42.

In some embodiments, the recombinant Fc-IL2v polypeptide complex according to the present disclosure comprises:
(i) a first polypeptide consisting of an amino acid sequence selected from the group consisting of SEQ ID NO:50, SEQ ID NO:51, SEQ ID NO:52 and SEQ ID NO:53; and
(ii) a second polypeptide consisting of the amino acid sequence of SEQ ID NO:41.

### Modifications Promoting Heterodimerization

As described herein, a Fc domain-IL2 variant (Fc-IL2v) polypeptide complex may comprise an Fc domain consisting of two subunits and comprising a modification promoting heterodimerization of two non-identical polypeptide chains as further described below, the recombinant Fc-IL2v polypeptide complex described herein may comprise an Fc domain subunit comprising a knob mutation and an Fc domain subunit comprising a hole mutation as herein before described.

A “modification promoting heterodimerization” is a manipulation of the peptide backbone or the post-translational modifications of a polypeptide that reduces or prevents the association of the polypeptide with an identical polypeptide to form a homodimer. A modification promoting heterodimerization as used herein particularly includes separate modifications made to each of two polypeptides desired to form a dimer, wherein the modifications are complementary to each other so as to promote association of the two polypeptides. For example, a modification promoting heterodimerization may alter the structure or charge of one or both of the polypeptides desired to form a dimer so as to make their association sterically or electrostatically favorable, respectively. Heterodimerization occurs between two non-identical polypeptides, such as two subunits of an Fc domain wherein further immunoconjugate components fused to each of the subunits (e.g. antigen binding moiety, effector moiety) are not the same. In the recombinant Fc-IL2v polypeptide complex according to the present invention, the modification promoting heterodimerization is in the Fc domain. In some embodiments the modification promoting heterodimerziation comprises an amino acid mutation, specifically an amino acid substitution. In a particular embodiment, the modification promoting heterodimerization comprises a separate amino acid mutation, specifically an amino acid substitution, in each of the two subunits of the Fc domain. The site of most extensive protein-protein interaction between the two polypeptide chains of a human IgG Fc domain is in the CH3 domain of the Fc domain. Thus, in one embodiment said modification is in the CH3 domain of the Fc domain. In a specific embodiment said modification is a knob-into-hole modification, comprising a knob modification in one of the two subunits of the Fc domain and a hole modification in the other one of the two subunits of the Fc domain.

The knob-into-hole technology is described e.g. in U.S. Pat. No. 5,731,168: U.S. Pat. No. 7,695,936: Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in formation of a disulfide bridge between the two subunits of the Fc region, further stabilizing the dimer (Carter, J Immunol Methods 248, 7-15 (2001)). Numbering of amino acid residues in the Fc region is according to the EU numbering system, also called the EU index, as described in Kabat et al., Sequences of Proteins of Immunological Interest. 5th Ed. Public Health Service. National Institutes of Health, Bethesda, MD. 1991. A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

In an alternative embodiment a modification promoting heterodimerization of two non-identical polypeptide chains comprises a modification mediating electrostatic steering effects, e.g. as described in WO 2009/089004. Generally, this method involves replacement of one or more amino acid residues at the interface of the two polypeptide chains by charged amino acid residues so that homodimer formation becomes electrostatically unfavorable but heterodimerization electrostatically favorable.

An IL-2 mutant having reduced binding affinity to the subunit of the IL-2 receptor may be fused to the carboxy-terminal amino acid of the subunit of the Fc domain comprising the knob modification. Without wishing to be bound by theory, fusion of the IL-2 mutant to the knob-containing subunit of the Fc domain will further minimize the generation of homodimeric immunoconjugates comprising two IL-2 mutant polypeptides (steric clash of two knob-containing polypeptides).

### Exemplary Fc Domain-IL2 Variant (Fc-IL2v) Polypeptide Complexes

In some aspect, provided is a recombinant Fc domain-IL2 variant (Fc-IL2v) polypeptide complex, comprising:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40, wherein the first polypeptide sequence is at least about 80%, 85%, 90%, 95%, 96%, 97%, 98%, 99% or 100% identical to a sequence selected from the group consisting of SEQ ID NO:43, SEQ ID NO:44, SEQ ID NO:47, SEQ ID NO:48, SEQ ID NO: 50, SEQ ID NO:51, SEQ ID NO:52 and SEQ ID NO 53.

In some aspects, provided is a recombinant Fc domain-IL2 variant (Fc-IL2v) polypeptide complex, comprising
(ii) a second polypeptide comprising a variant CH2-CH3 region comprising G329 according to EU numbering, wherein the second polypeptide sequence is at least about 80%, 85%, 90%, 95%, 96%, 97%, 98%, 99% or 100% identical to the sequence of SEQ ID NO:41 or SEQ ID NO:42.

In specific aspects, provided is a Fc-IL2v polypeptide complex comprising a first polypeptide sequence comprising or consisting of an amino acid sequence selected from the group consisting of SEQ ID NO:43, SEQ ID NO:44, SEQ ID NO:47, SEQ ID NO:48, SEQ ID NO:50, SEQ ID NO:51, SEQ ID NO:52 and SEQ ID NO 53.

In specific aspects, provided is a Fc-IL2v polypeptide complex comprising a second polypeptide sequence comprising or consisting of an amino acid sequence selected from the group consisting of SEQ ID NO:41 and SEQ ID NO:42.

In some aspects, provided is a recombinant Fc domain-IL2 variant (Fc-IL2v) polypeptide complex, comprising:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40, wherein the first polypeptide sequence is at least about 80%, 85%, 90%, 95%, 96%, 97%, 98%, 99% or 100% identical to a sequence selected from the group consisting of SEQ ID NO:43, SEQ ID NO:44, SEQ ID NO:47, SEQ ID NO:48, SEQ ID NO: 50, SEQ ID NO:51, SEQ ID NO:52 and SEQ ID NO 53; and
(ii) a second polypeptide comprising a variant CH2-CH3 region comprising G329 according to EU numbering, wherein the second polypeptide sequence is at least about 80%, 85%, 90%, 95%, 96%, 97%, 98%, 99% or 100% identical to the sequence of SEQ ID NO:41 or SEQ ID NO:42.

In specific aspects, provided is Fc-IL2v polypeptide complex comprising:
(i) a first polypeptide comprising an amino acid sequence selected from the group consisting of SEQ ID NO:50, SEQ ID NO:51, SEQ ID NO:52, and SEQ ID NO:53; and
(ii) a second polypeptide comprising an amino acid sequence of SEQ ID NO:41.

In specific aspects, provided is Fc-IL2v polypeptide complex comprising:
(i) a first polypeptide comprising an amino acid sequence selected from the group consisting of SEQ ID NO:43, SEQ ID NO:44, SEQ ID NO:47, and SEQ ID NO:48; and
(ii) a second polypeptide comprising an amino acid sequence of SEQ ID NO:42.

In a preferred aspect, provided is Fc-IL2v polypeptide complex comprising:
(i) a first polypeptide comprising the amino acid sequence SEQ ID NO:44; and
(ii) a second polypeptide comprising an amino acid sequence of SEQ ID NO:42.

In a preferred aspect, provided is Fc-IL2v polypeptide complex comprising:
(i) a first polypeptide comprising the amino acid sequence SEQ ID NO:51; and
(ii) a second polypeptide comprising an amino acid sequence of SEQ ID NO:41.
   wherein the recombinant Fc-IL2v polypeptide complex does not comprise an antigen binding moiety.

In some embodiments, the recombinant Fc-IL2v polypeptide complex according to the present disclosure consists of:
(i) a first polypeptide consisting of an amino acid sequence selected from the group consisting of SEQ ID NO:43, SEQ ID NO:44, SEQ ID NO:47, and SEQ ID NO:48; and
(ii) a second polypeptide consisting of the amino acid sequence of SEQ ID NO:42.

In some embodiments, the recombinant Fc-IL2v polypeptide complex according to the present disclosure consists of:
(i) a first polypeptide consisting of an amino acid sequence selected from the group consisting of SEQ ID NO:50, SEQ ID NO:51, SEQ ID NO:52 and SEQ ID NO: 53; and
(ii) a second polypeptide consisting of the amino acid sequence of SEQ ID NO:41,
   wherein the recombinant Fc-IL2v polypeptide complex does not comprise an antigen binding moiety.

In a preferred embodiment, the recombinant Fc-IL2v polypeptide complex according to the present disclosure consists of:
(i) a first polypeptide consisting of the amino acid sequence of SEQ ID NO:43 or SEQ ID NO: 44; and
(ii) a second polypeptide consisting of the amino acid sequence of SEQ ID NO:42.

Hence, in a preferred embodiment of the invention the recombinant Fc-IL2v polypeptide complex used in the above described combination treatments and medical uses of different diseases is a Fc-IL2v polypeptide complex characterized in consisting of the polypeptide sequences of SEQ ID NO: 42 and SEQ ID NO: 44 or SEQ ID NO: 42 and SEQ ID NO:43.

### PD-1-Targeted Fc-IL2v Polypeptide Complexes

In some aspects, the recombinant Fc-IL2v polypeptide complex comprises at least one antigen binding moiety that binds to PD-1 (“PD1-targeted Fc-IL2v polypeptide complex”). PD-1-targeted Fc-IL2v polypeptide complexes may be prepared as described in the Examples of WO 2018/184964. An important negative co-stimulatory signal regulating T cell activation is provided by programmed death-1 receptor (PD-1) (CD279), and its ligand binding partners PD-L1 (B7-H1, CD274) and PD-L2 (B7-DC, CD273). The negative regulatory role of PD-1 was revealed by PD-1 knock outs (Pdcd1−/−), which are prone to autoimmunity. Nishimura et al., Immunity 11:141-51 (1999); Nishimura et al., Science 291:319-22 (2001). PD-1 is related to CD28 and CTLA-4, but lacks the membrane proximal cysteine that allows homodimerization. The cytoplasmic domain of PD-1 contains an immunoreceptor tyrosine-based inhibition motif (ITIM, V/IxYxxL/V). PD-1 only binds to PD-L1 and PD-L2. Freeman et al., J. Exp. Med. 192:1-9 (2000): Dong et al., Nature Med. 5:1365-1369 (1999): Latchman et al., Nature Immunol. 2:261-268 (2001): Tseng et al., J. Exp. Med. 193:839-846 (2001).

PD-1 can be expressed on T cells, B cells, natural killer T cells, activated monocytes and dendritic cells (DCs). PD-1 is expressed by activated, but not by unstimulated human CD4⁺ and CD8⁺ T cells, B cells and myeloid cells. This stands in contrast to the more restricted expression of CD28 and CTLA-4 (Nishimura et al., Int. Immunol. 8:773-80 (1996); Boettler et al., J. Virol. 80:3532-40 (2006)). There are at least 4 variants of PD-1 that have been cloned from activated human T cells, including transcripts lacking (i) exon 2, (ii) exon 3, (iii) exons 2 and 3 or (iv) exons 2 through 4 (Nielsen et al., Cell. Immunol. 235: 109-16 (2005)). With the exception of PD-1 Δex3, all variants are expressed at similar levels as full length PD-1 in resting peripheral blood mononuclear cells (PBMCs). Expression of all variants is significantly induced upon activation of human T cells with anti-CD3 and anti-CD28. The PD-1 Δex3 variants lacks a transmembrane domain, and resembles soluble CTLA-4, which plays an important role in autoimmunity (Ueda et al., Nature 423:506-11 (2003)). This variant is enriched in the synovial fluid and sera of patients with rheumatoid arthritis. Wan et al., J. Immunol. 177:8844-50 (2006).

The two PD-1 ligands differ in their expression patterns. PD-L1 is constitutively expressed on mouse T and B cells. CDs, macrophages, mesenchymal stem cells and bone marrow-derived mast cells (Yamazaki et al., J. Immunol. 169:5538-45 (2002)). PD-L1 is expressed on a wide range of non-hematopoietic cells (e.g., cornea, lung, vascular epithelium, liver non-parenchymal cells, mesenchymal stem cells, pancreatic islets, placental synctiotrophoblasts, keratinocytes, etc.) (Keir et al., Annu. Rev. Immunol. 26:677-704 (2008)), and is upregulated on a number of cell types after activation. Both type I and type II interferons IFN's) upregulate PD-L1 (Eppihimer et al., Microcirculation 9:133-45 (2002): Schreiner et al., J. Neuroimmunol. 155:172-82 (2004)). PD-L1 expression in cell lines is decreased when My D88, TRAF6 and MEK are inhibited (Liu et al., Blood 110:296-304 (2007)). JAK2 has also been implicated in PD-L1 induction (Lee et al., FEBS Lett. 580): 755-62 (2006); Liu et al., Blood 110:296-304 (2007)). Loss or inhibition of phosphatase and tensin homolog (PTEN), a cellular phosphatase that modified phosphatidylinositol 3-kinase (PI3K) and Akt signaling, increased post-transcriptional PD-L1 expression in cancers (Parsa et al., Nat. Med. 13:84-88 (2007)).

PD-L2 expression is more restricted than PD-L1. PD-L2 is inducibly expressed on DCs, macrophages, and bone marrow-derived mast cells. PD-L2 is also expressed on about half to two-thirds of resting peritoneal B1 cells, but not on conventional B2 B cells (Zhong et al., Eur. J. Immunol. 37:2405-10 (2007)). PD-L2+ B1 cells bind phosphatidylcholine and may be important for innate immune responses against bacterial antigens. Induction of PD-L2 by IFN-gamma is partially dependent upon NF-κB (Liang et al., Eur. J. Immunol. 33:2706-16 (2003)). PD-L2 can also be induced on monocytes and macrophages by GM-CF. IL-4 and IFN-gamma (Yamazaki et al., J. Immunol. 169: 5538-45 (2002): Loke et al., PNAS 100:5336-41 (2003)).

PD-1 signaling typically has a greater effect on cytokine production than on cellular proliferation, with significant effects on IFN-gamma. TNF-alpha and IL-2 production. PD-1 mediated inhibitory signaling also depends on the strength of the TCR signaling, with greater inhibition delivered at low levels of TCR stimulation. This reduction can be overcome by costimulation through CD28 (Freeman et al., J. Exp. Med. 192:1027-34 (2000)) or the presence of IL-2 (Carter et al., Eur. J. Immunol. 32:634-43 (2002)).

Evidence is mounting that signaling through PD-L1 and PD-L2 may be bidirectional. That is, in addition to modifying TCR or BCR signaling, signaling may also be delivered back to the cells expressing PD-L1 and PD-L2. While treatment of dendritic cells with a naturally human anti-PD-L2 antibody isolated from a patient with Waldenstrom's macroglobulinemia was not found to upregulate MHC II or B7 costimulatory molecules, such cells did produce greater amount of proinflammatory cytokines, particularly TNF-alpha and IL-6, and stimulated T cell proliferation (Nguyen et al., J. Exp. Med. 196:1393-98 (2002)). Treatment of mice with this antibody also (1) enhanced resistance to transplanted b16 melanoma and rapidly induced tumor-specific CTL (Radhakrishnan et al., J. Immunol. 170:1830-38 (2003): Radhakrishnan et al., Cancer Res. 64:4965-72 (2004): Heckman et al., Eur. J. Immunol. 37:1827-35 (2007)): (2) blocked development of airway inflammatory disease in a mouse model of allergic asthma (Radhakrishnan et al., J. Immunol. 173:1360-65 (2004): Radhakrishnan et al., J. Allergy Clin. Immunol. 116:668-74 (2005)).

Further evidence of reverse signaling into dendritic cells (“DC's”) results from studies of bone marrow derived DC's cultured with soluble PD-1 (PD-1 EC domain fused to Ig constant region—“s-PD-1”) (Kuipers et al., Eur. J. Immunol. 36:2472-82 (2006)). This sPD-1 inhibited DC activation and increased IL-10 production, in a manner reversible through administration of anti-PD-1.

Additionally, several studies show a receptor for PD-L1 or PD-L2 that is independent of PD-1. B7.1 has already been identified as a binding partner for PD-L1 (Butte et al., Immunity 27:111-22 (2007)). Chemical crosslinking studies suggest that PD-L1 and B7.1 can interact through their IgV-like domains. B7.1:PD-L1 interactions can induce an inhibitory signal into T cells. Ligation of PD-L1 on CD4⁺ T cells by B7.1 or ligation of B7.1 on CD4 T cells by PD-L1 delivers an inhibitory signal. T cells lacking CD28 and CTLA-4 show decreased proliferation and cytokine production when stimulated by anti-CD3 plus B7.1 coated beads. In T cells lacking all the receptors for B7.1 (i.e., CD28, CTLA-4 and PD-L1). T cell proliferation and cytokine production were no longer inhibited by anti-CD3 plus B7.1 coated beads. This indicates that B7.1 acts specifically through PD-L1 on the T-cell in the absence of CD28 and CTLA-4. Similarly, T cells lacking PD-1 showed decreased proliferation and cytokine production when stimulated in the presence of anti-CD3 plus PD-L1 coated beads, demonstrating the inhibitory effect of PD-L1 ligation on B7.1 on T cells. When T cells lacking all known receptors for PD-L1 (i.e., no PD-1 and B7.1). T cell proliferation was no longer impaired by anti-CD3 plus PD-L1 coated beads. Thus, PD-L1 can exert an inhibitory effect on T cells either through B7.1 or PD-1.

The direct interaction between B7.1 and PD-L1 suggests that the current understanding of costimulation is incomplete, and underscores the significance to the expression of these molecules on T cells. Studies of PD-L1^{-/-} T cells indicate that PD-L1 on T cells can downregulate T cell cytokine production (Latchman et al., Proc. Natl. Acad. Sci. USA 101:10691-96 (2004)). Because both PD-L1 and B7.1 are expressed on T cells, B cells, DCs and macrophages, there is the potential for directional interactions between B7.1 and PD-L1 on these cells types. Additionally, PD-L1 on non-hematopoietic cells may interact with B7.1 as well as PD-1 on T cells, raising the question of whether PD-L1 is involved in their regulation. One possible explanation for the inhibitory effect of B7.1:PD-L1 interaction is that T cell PD-L1 may trap or segregate away APC B7.1 from interaction with CD28.

As a result, the antagonism of signaling through PD-L1, including blocking PD-L1 from interacting with either PD-1, B7.1 or both, thereby preventing PD-L1 from sending a negative co-stimulatory signal to T-cells and other antigen presenting cells is likely to enhance immunity in response to infection (e.g., acute and chronic) and tumor immunity. In addition, the anti-PD-L1 antibodies of the present invention, may be combined with antagonists of other components of PD-1:PD-L1 signaling, for example, antagonist anti-PD-1 and anti-PD-L2 antibodies.

The ability of IL-2 to expand and activate lymphocytes and natural killer (NK) cells underlies the anti-tumor activity of IL-2. IL-2 mutants designed to eliminate the binding of IL-2 to IL-2a subunit (CD25) overcome the limitations of IL-2 and as part of a tumor-targeted IL-2 variant immunoconjugate, such as a CEA-targeted IL-2 variant immunoconjugate or a FAP-targeted IL-2 variant immunoconjugate, have been shown to be able to eliminate tumor cells.

The recombinant Fc-IL2v polypeptide complex used in the combination therapy described herein may comprise an antibody which binds to PD-1 on PD-1 expressing immune cells, particularly T cells, or in a tumor cell environment, or an antigen binding fragment thereof, and an IL-2 mutant, particularly a mutant of human IL-2, having reduced binding affinity to the α-subunit of the IL-2 receptor (as compared to wild-type IL-2, e.g. human IL-2 shown as SEQ ID NO: 40), such as an IL-2 comprising: i) one, two or three amino acid substitution(s) at one, two or three position(s) selected from the positions corresponding to residues 42, 45 and 72 of human IL-2 shown as SEQ ID NO: 40, for example three substitutions at three positions, for example the specific amino acid substitutions F42A, Y45A and L72G; or ii) the features as set out in i) plus an amino acid substitution at a position corresponding to residue 3 of human IL-2 shown as SEQ ID NO: 40, for example the specific amino acid substitution T3A; or iii) four amino acid substitutions at positions corresponding to residues 3, 42, 45 and 72 of human IL-2 shown as SEQ ID NO: 40, for example the specific amino acid substitutions T3A. F42A, Y45A and L72G.

The recombinant Fc-IL2v polypeptide complex used in the combination therapy described herein may comprise a heavy chain variable domain and a light chain variable domain of an antibody which binds to PD-1 presented on immune cells, particularly T cells, or in a tumor cell environment and an Fc domain consisting of two subunits and comprising a modification promoting heterodimerization of two non-identical polypeptide chains, and an IL-2 mutant, particularly a mutant of human IL-2, having reduced binding affinity to the α-subunit of the IL-2 receptor (as compared to wild-type IL-2, e.g. human IL-2 shown as SEQ ID NO: 40), such as an IL-2 comprising: i) one, two or three amino acid substitution(s) at one, two or three position(s) selected from the positions corresponding to residues 42, 45 and 72 of human IL-2 shown as SEQ ID NO: 40, for example three substitutions at three positions, for example the specific amino acid substitutions F42A, Y45A and L72G; or ii) the features as set out in i) plus an amino acid substitution at a position corresponding to residue 3 of human IL-2 shown as SEQ ID NO: 40, for example the specific amino acid substitution T3A; or iii) four amino acid substitutions at positions corresponding to residues 3, 42, 45 and 72 of human IL-2 shown as SEQ ID NO: 40, for example the specific amino acid substitutions T3A, F42A, Y45A and L72G.

A Fc-IL2v polypeptide complex used in the combination therapy may comprise a) a heavy chain variable domain VH of SEQ ID NO: 36 and a light chain variable domain VL of SEQ ID NO: 37, and the polypeptide sequence of SEQ ID NO: 38, or a heavy chain variable domain VH of SEQ ID NO: 36 and a light chain variable domain VL of SEQ ID NO: 37, and the polypeptide sequence of SEQ ID NO: 39, or c) a polypeptide sequence of SEQ ID NO: 54 or SEQ ID NO: 55 or SEQ ID NO: 56, or d) the polypeptide sequences of SEQ ID NO: 58, and SEQ ID NO: 59 and SEQ ID NO: 60.

In some embodiments, the recombinant Fc-IL2v polypeptide complex used in the combination therapy comprises the polypeptide sequences of SEQ ID NO: 54, SEQ ID NO: 55 and SEQ ID NO: 56.

These PD1-targeted Fc-IL2v polypeptide complexes, along with their component parts of antigen binding moieties. Fc domains and effector moieties, are described as examples of the immunoconjugates described in WO 2018/184964. For example, the particular immunoconjugates “PD-1-targeted IgG-IL-2 qm fusion protein” based on the anti-CEA antibody CH1A1A 98/99 2F1 and IL-2 quadruple mutant (qm) are described in e.g., Examples 1 and 2 of WO 2018/184964.

In preferred embodiments. PD-1 targeting of the recombinant Fc-IL2v polypeptide complex may be achieved by targeting PD-1, as described in WO 2018/1184964. PD-1-targeting may be achieved with an anti-PD-1 antibody or an antigen binding fragment thereof. An anti-PD-1 antibody may comprise a heavy chain variable region sequence that is at least about 80%, 85%, 90%, 95%, 96%, 97%, 98%, 99% or 100% identical to the sequence of SEQ ID NO: 36 or a variant thereof that retains functionality. An anti-PD-1 antibody may comprise a light chain variable region sequence that is at least about 80%, 85%, 90%, 95%, 96%, 97%, 98%, 99% or 100% identical to the sequence of SEQ ID NO: 37 or a variant thereof that retains functionality. An anti-PD-1 antibody may comprise a heavy chain variable region sequence that is at least about 80%, 85%, 90%, 95%, 96%, 97%, 98%, 99% or 100% identical to the sequence of SEQ ID NO: 36, or a variant thereof that retains functionality, and a light chain variable region sequence that is at least about 80%, 85%, 90%, 95%, 96%, 97%, 98%, 99% or 100% identical to the sequence of SEQ ID NO: 37, or a variant thereof that retains functionality. An anti-PD-1 antibody may comprise the heavy chain variable region sequence of SEQ ID NO: 36 and the light chain variable region sequence of SEQ ID NO: 37.

The recombinant Fc-IL2v polypeptide complex may comprise a polypeptide sequence selected from the group consisting of SEQ ID NO: 54. SEQ ID NO: 55 and SEQ ID NO: 56, or a variant thereof that retains functionality (e.g. the IL2v comprises a further amino acid substitution at a position corresponding to 126 of human IL-2 (shown in SEQ ID NO:40), wherein the amino acid substitution is Q126T). The recombinant Fc-IL2v polypeptide complex may comprise a polypeptide sequence wherein a Fab heavy chain specific for PD-1 shares a carboxy-terminal peptide bond with an Fc domain subunit comprising a hole modification. The recombinant Fc-IL2v polypeptide complex may comprise the polypeptide sequence of SEQ ID NO: 54 or SEQ ID NO: 55, or a variant thereof that retains functionality. The recombinant Fc-IL2v polypeptide complex may comprise a Fab light chain specific for PD-1. The recombinant Fc-IL2v polypeptide complex may comprise the polypeptide sequence of SEQ ID NO: 56, or a variant thereof that retains functionality. The polypeptides may be covalently linked, e.g., by a disulfide bond. The Fc domain polypeptide chains may comprise the amino acid substitutions L234A, L235A, and P329G (which may be referred to as LALA P329G).

As described in WO 2018/184964, the recombinant Fc-IL2v polypeptide complex may be a PD-1-targeted IgG-IL-2 qm fusion protein having the sequences shown as SEQ ID NOs: 54, 55, 56 (as described in e.g. Example 1 of WO 2018/184964). The recombinant Fc-IL2v polypeptide complex having the sequences shown as SEQ ID NOs: 54, 55, 56 is referred to herein as “PD1-IL2v”. The recombinant Fc-IL2v polypeptide complex having the sequences shown as SEQ ID NOs: 58, 59, 60 is referred to herein as “muPD1-IL2v”, which is a murine surrogate.

The recombinant Fc-IL2v polypeptide complex used in the combination therapy described herein may comprise an antibody which binds to an antigen presented on immune cells, particularly T cells, or in a tumor cell environment, and an IL-2 mutant having reduced binding affinity to the subunit of the IL-2 receptor. The recombinant Fc-IL2v polypeptide complex may essentially consist of an antibody which binds to PD-1 presented on immune cells, particularly T cells, or in a tumor cell environment, and an IL-2 mutant having reduced binding affinity to the subunit of the IL-2 receptor. The antibody may be an IgG antibody, particularly an IgG1 antibody. The recombinant Fc-IL2v polypeptide complex may comprise a single IL-2 mutant having reduced binding affinity to the subunit of the IL-2 receptor (i.e. not more than one IL-2 mutant moiety is present).

In one embodiment, the recombinant Fc-IL2v polypeptide complex comprises or consist of a polypeptide comprising an amino acid sequence that is at least about 80%, 85%, 90%, 95%, 96%, 97%, 98%, 99% or 100% identical to the sequence of SEQ ID NO:322, a polypeptide comprising an amino acid sequence that is at least about 80%, 85%, 90%, 95%, 96%, 97%, 98%, 99% or 100% identical to the sequence of SEQ ID NO: 323, a polypeptide comprising an amino acid sequence that is at least about 80%, 85%, 90%, 95%, 96%, 97%, 98%, 99% or 100% identical to the sequence of SEQ ID NO:324 and a polypeptide comprising an amino acid sequence that is at least about 80%, 85%, 90%, 95%, 96%, 97%, 98%, 99% or 100% identical to the sequence of SEQ ID NO:325.

In a preferred embodiment, the recombinant Fc-IL2v polypeptide complex consist of a polypeptide comprising or consisting of the amino acid sequence of SEQ ID NO:322, a polypeptide comprising or consisting of the amino acid sequence of SEQ ID NO:323, a polypeptide comprising or consisting of the amino acid sequence of SEQ ID NO:324 and a polypeptide comprising or consisting of the amino acid sequence of SEQ ID NO:325.

In one embodiment, the recombinant Fc-IL2v polypeptide complex comprises or consist of a polypeptide comprising an amino acid sequence that is at least about 80%, 85%, 90%, 95%, 96%, 97%, 98%, 99% or 100% identical to the sequence of SEQ ID NO:326, a polypeptide comprising an amino acid sequence that is at least about 80%, 85%, 90%, 95%, 96%, 97%, 98%, 99% or 100% identical to the sequence of SEQ ID NO: 327, and a polypeptide comprising an amino acid sequence that is at least about 80%, 85%, 90%, 95%, 96%, 97%, 98%, 99% or 100% identical to the sequence of SEQ ID NO:328.

In a preferred embodiment, the recombinant Fc-IL2v polypeptide complex consist of a polypeptide comprising or consisting of the amino acid sequence of SEQ ID NO:326, a polypeptide comprising or consisting of the amino acid sequence of SEQ ID NO:327, and a polypeptide comprising or consisting of the amino acid sequence of SEQ ID NO:328.

### Membrane-Anchored Antigen Binding (MAB) Polypeptides and MAB Polypeptide Complexes

Herein, a “membrane-anchored antigen binding polypeptide” or “MAB polypeptide” refers to a polypeptide comprising an antigen binding moiety that binds to a variant CH2-CH3 region according to the present disclosure comprising G329 according to EU numbering and a transmembrane domain.

A “MAB polypeptide complex” according to the present disclosure comprises at least two MAB polypeptides according to the present disclosure, wherein the two MAB polypeptides form an antigen binding moiety that binds to a variant CH2-CH3 region according to the present disclosure comprising G329 according to EU numbering. At least one of the at least two MAB polypeptides comprise a transmembrane domain.

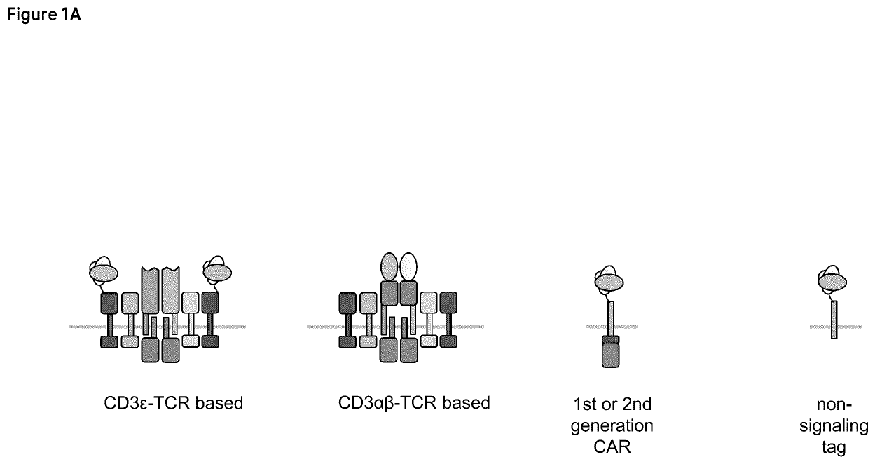

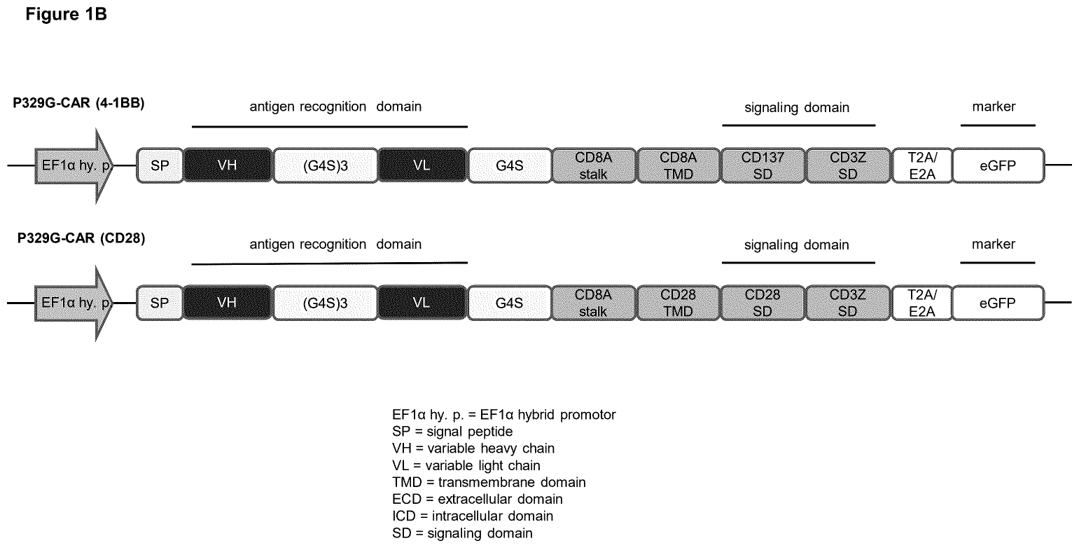

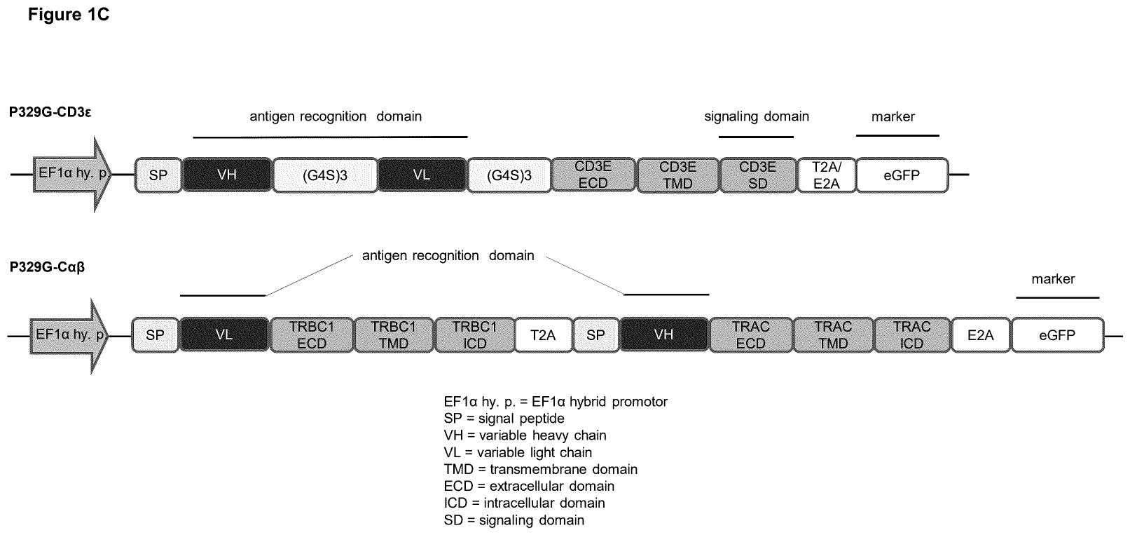

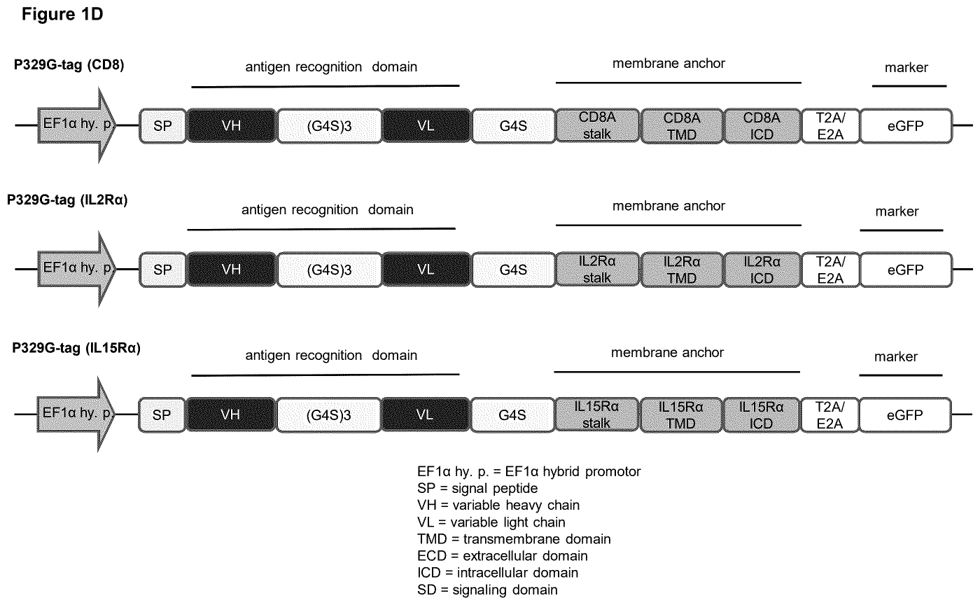

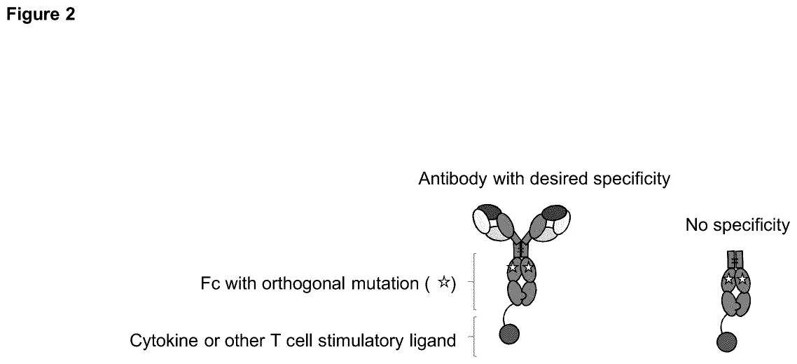

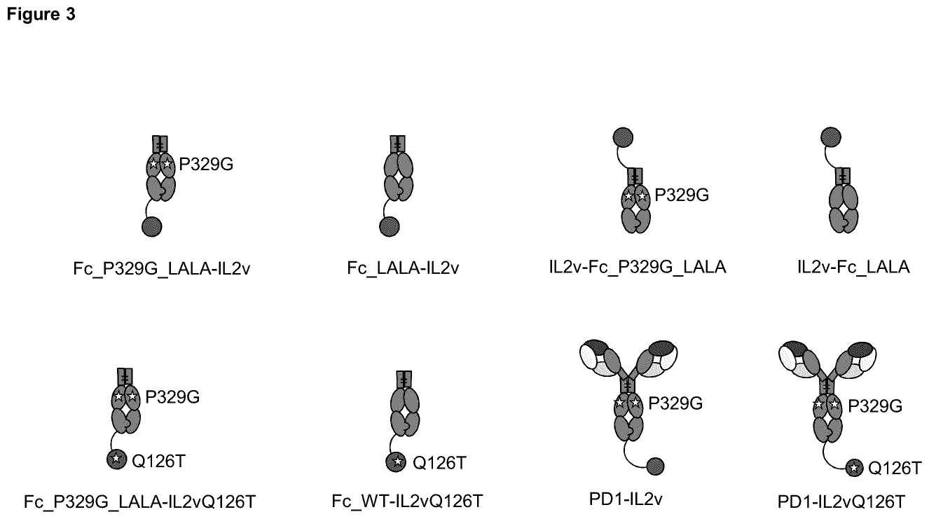

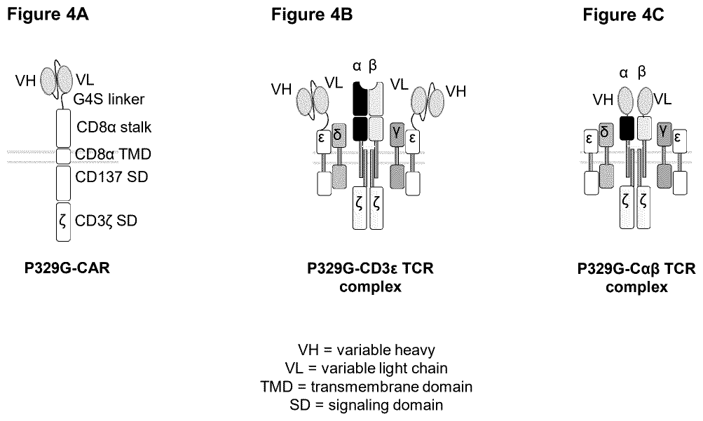

Exemplary configurations of MAB polypeptide (complexes) are depicted in FIGS. 1 and 4.

For example FIG. 1A depicts two MAB polypeptides according to the present disclosure composed of an antigen binding moiety that bind to a variant CH2-CH3 region comprising G329 according to EU numbering fused to the CD3-TCR complex polypeptide CD38, wherein the two MAB polypeptides are integrated into a TCR complex.

For example FIG. 1B depicts two MAB polypeptides which form a MAB polypeptide complex according to the present disclosure composed of an antigen binding moieties that bind to a variant CH2-CH3 region comprising G329 according to EU numbering fused to the CD3-TCR complex polypeptides TCRα and TCRβ.

For example FIG. 1C depicts a MAB polypeptide according to the present disclosure composed of an antigen binding moiety that bind to a variant CH2-CH3 region comprising G329 according to EU numbering fused to the transmembrane and intracellular signalling domains of a chimeric antigen receptor (CAR).

For example FIG. 1D depicts a MAB polypeptide according to the present disclosure composed of an antigen binding moiety that bind to a variant CH2-CH3 region comprising G329 according to EU numbering fused to a transmembrane domain.

The MAB polypeptide (complex) of the present disclosure comprise an antigen-binding moiety, or a component thereof as further described below. The essential function of the antigen-binding moiety is to provide for binding to a variant CH2-CH3 domain of the recombinant Fc-IL2v polypeptide complex, as described herein above.

The MAB polypeptide (complex) of the present disclosure further comprises a transmembrane domain as further described below. The essential function of the transmembrane domain is to anchor the MAB polypeptide (complex) in the plasma membrane of the MAB expressing cell (e.g. a T cell). Hence, the transmembrane domain confines the activity of the recombinant Fc-IL2v polypeptide complex as described herein above to the cell of interest (e.g. a recombinant MAB polypeptide and/or a recombinant MAB complex expressing T cell). In the context of the present invention any transmembrane domain of a transmembrane protein as laid down among others by the CD-nomenclature may be used to generate the antigen binding receptors of the invention. Further specific transmembrane domains are described herein below.

In some aspects the MAB polypeptide further comprises intracellular signaling domains as further described below.

The MAB polypeptide include but are not limited to chimeric antigen receptors (CARs), recombinant T cell receptors (TCRs), and non-signaling tags as shown in FIG. 1.

### P329G Antigen-Binding Moieties

“Antigen-binding moieties” include antibodies (i.e. immunoglobulins (Igs)), and antigen-binding fragments and derivatives thereof. In some embodiments, an antigen-binding moiety according to the present disclosure comprises, or consists of, a monoclonal antibody, a monospecific antibody, a multispecific (e.g., bispecific, trispecific, etc.) antibody, a variable fragment (Fv) moiety, a single-chain Fv (scFv) moiety, a fragment antigen-binding (Fab) moiety, a single-chain Fab moiety (scFab), a crossFab moiety, a Fab′ moiety, a Fab-SH moiety, a F(ab′)₂ moiety, a diabody moiety, a triabody moiety, an scFv-Fc moiety, a minibody moiety, a heavy chain only antibody (HCAb) moiety, or a single domain antibody (dAb, VHH) moiety.

Antigen-binding moieties according to the present disclosure also include further target antigen-binding peptides/polypeptides such as peptide aptamers, thioredoxins, anticalins, Kunitz domains, avimers, knottins, fynomers, atrimers, DARPins, affibodys, affilins, armadillo repeat proteins (ArmRPs), OBodys and adnectins (reviewed e.g. in Reverdatto et al., Curr Top Med Chem. 2015; 15 (12): 1082-1101, which is hereby incorporated by reference in its entirety (see also e.g. Boersma et al., J Biol Chem (2011) 286:41273-85 and Emanuel et al., Mabs (2011) 3:38-48)). Antigen-binding moieties according to the present disclosure also include target antigen-binding nucleic acids, e.g. nucleic acid aptamers (reviewed, for example, in Zhou and Rossi Nat Rev Drug Discov. 2017 16 (3): 181-202). Antigen-binding moieties according to the present disclosure also include target antigen-binding small molecules (e.g. low molecular weight (<1000 daltons, typically between ˜300-700 daltons) organic compounds).

The antigen-binding moiety of the MAB polypeptides of the present disclosure are capable of binding to a variant Fc domain according to the present disclosure. Antigen-binding moieties that are capable of binding to a variant Fc domain according to the present disclosure may also be described as antigen-binding moieties that bind to a variant Fc domain according to the present disclosure.

The antigen-binding moieties described herein preferably display specific binding to a variant Fc domain according to the present disclosure. As used herein. “specific binding” refers to binding which is selective for the target antigen, and which can be discriminated from non-specific binding to non-target antigen. An antigen-binding moiety that specifically binds to a given target antigen preferably binds the target antigen with greater affinity, and/or with greater duration than it binds to other, non-target antigens.

The ability of a given moiety to bind specifically to a given target antigen can be determined by analysis according to methods known in the art, such as by ELISA. Surface Plasmon Resonance (SPR; see e.g. Hearty et al., Methods Mol Biol (2012) 907: 411-442), Bio-Layer Interferometry (BLI; see e.g. Lad et al., (2015) J Biomol Screen 20 (4): 498-507), flow cytometry, or by a radiolabeled antigen-binding assay (RIA) enzyme-linked immunosorbent assay. Through such analysis binding to a given target antigen can be measured and quantified. In some embodiments, the level of binding may be the response detected in a given assay.

In some embodiments, the antigen-binding moiety described herein binds to a variant Fc domain according to the present disclosure with an affinity (e.g. determined by SPR or BLI) in the micromolar range, i.e. K_{D}=9.9×10⁻⁴ to 1×10⁻⁶ M. In some embodiments, the antigen-binding moiety described herein binds to a variant Fc domain according to the present disclosure with sub-micromolar affinity, i.e. K_{D}<1×10⁻⁶ M. In some embodiments, the antigen-binding moiety described herein binds to a variant Fc domain according to the present disclosure with an affinity in the nanomolar range, i.e. K_{D}=9.9×10⁻⁷ to 1×10⁻⁹ M. In some embodiments, the antigen-binding moiety described herein binds to a variant Fc domain according to the present disclosure with sub-nanomolar affinity, i.e. K_{D}<1×10⁻⁹ M. In some embodiments, the antigen-binding moiety described herein binds to a variant Fc domain according to the present disclosure with an affinity in the picomolar range, i.e. K_{D}=9.9×10⁻¹⁰ to 1×10⁻¹² M. In some embodiments, the antigen-binding moiety described herein binds to a variant Fc domain according to the present disclosure with sub-picomolar affinity, i.e. K_{D}<1×10⁻¹² M.

The antigen-binding moieties of the recombinant MAB polypeptides according to the present disclosure preferably do not display specific binding to a reference Fc domain according to the present disclosure. In some embodiments, the antigen-binding moiety does not bind, or displays substantially no binding, to a reference Fc domain according to the present disclosure.

An antigen-binding moiety that “does not bind” or that “displays substantially no binding” to a given antigen displays a level of binding to the given antigen which is similar to the level of binding to an antigen that the antigen-binding moiety is known not to bind, or known to not to bind specifically, e.g. a non-target antigen. In some embodiments, the level of binding of an antigen-binding moiety that does not bind, or that displays substantially no binding, to a given antigen is ≥0.5 times and ≤2 times, e.g. one of ≥0.75 times and ≤1.5 times, ≥0.8 times and ≤1.4 times, ≥0.85 times and ≤1.3 times, ≥0.9 times and ≤1.2 times, ≥0.95 times and ≤1.1 times the level of binding displayed by the antigen-binding moiety to an antigen that the antigen-binding moiety is known not to bind, or known to not to bind specifically, e.g. a non-target antigen.

In some embodiments, the level of binding of the antigen-binding moiety to a reference Fc domain according to the present disclosure is ≤10% of the binding of the antigen-binding moiety to a variant Fc domain according to the present disclosure as determined e.g. by ELISA, SPR, BLI or RIA. In some embodiments, the antigen-binding moiety binds to a reference Fc domain according to the present disclosure with an equilibrium dissociation constant (K_{D}; e.g. determined by SPR or BLI) that is at least 0.1 order of magnitude greater than the K_{D} of the antigen-binding moiety for a variant Fc domain according to the present disclosure.

An antigen-binding moiety according to the present disclosure may be, or may comprise, an antigen-binding peptide/polypeptide, or an antigen-binding peptide/polypeptide complex. An antigen-binding moiety may comprise more than one peptide/polypeptide that together form an antigen-binding domain. The peptides/polypeptides may associate covalently or non-covalently. In some embodiments, the peptides/polypeptides form part of a larger polypeptide comprising the peptides/polypeptides (e.g. in the case of an scFv moiety comprising a VH region and a VL region, or in the case of a scFab moiety comprising VH-CH1 and VL-CL).

In some embodiments, the antigen-binding moiety of the present disclosure comprises an antibody heavy chain variable (VH) region and an antibody light chain variable (VL) region of an antibody capable of binding to a given target antigen. In some embodiments, the antigen-binding moiety comprises, or consists of, an Fv moiety formed by the VH region and a VL region of an antibody capable of binding to a given target antigen. In some embodiments, the VH region and a VL region may be provided in the same polypeptide, and joined by a linker sequence. In some embodiments, the antigen-binding moiety comprises, or consists of, an scFv moiety that binds to a given target antigen.

Antigen-binding moieties of the present disclosure generally comprise six complementarity-determining regions CDRs; three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antigen-binding moiety, which is the part of the moiety that binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as (i) the Kabat system, described in Kabat et al., Sequences of Proteins of Immunological Interest, 5th Ed. Public Health Service, National Institutes of Health, Bethesda. MD (1991); (ii) the Chothia system, described in Chothia et al., J. Mol. Biol. 196:901-917 (1987); and (iii) the international IMGT (ImMunoGeneTics) information system (LeFranc et al., Nucleic Acids Res. (2015) 43 (Database issue); D413-22), which uses the IMGT V-DOMAIN numbering rules as described in Lefranc et al., Dev. Comp. Immunol. (2003) 27:55-77.

The CDRs and FRs of the VH regions and VL regions of the antigen-binding moieties described herein are defined according to the Kabat system.

In some embodiments, the antigen-binding moiety comprises the CDRs of an antigen-binding moiety that binds to a variant Fc domain according to the present disclosure. In some embodiments, the antigen-binding moiety comprises the FRs of an antigen-binding moiety that binds to a variant Fc domain according to the present disclosure. In some embodiments, the antigen-binding moiety comprises the CDRs and the FRs of an antigen-binding moiety that binds to a variant Fc domain according to the present disclosure. That is, in some embodiments, the antigen-binding moiety comprises the VH region and the VL region of an antigen-binding moiety that binds to a variant Fc domain according to the present disclosure.

Wessels et al. Bioanal. (2017) 9 (11): 849-59 describes the identification of an antibody that binds to antibodies comprising an Fc domain derived from human IgG1 comprising P329G, but that does not bind to antibodies comprising the equivalent Fc domain lacking the P329G substitution. The antibody also binds to antibodies having a hIgG1-derived Fc region comprising P329G and further comprising L234A and L235A. Darowski et al., Protein Eng. Des. Sel. (2019) 32 (5): 207-218 and Stock et al., Journal for ImmunoTherapy of Cancer (2022) 10:e005054 provide the structure of the anti-P329G Fab with Fc comprising P329G, L234A and L235A. The anti-P329G Fab interacts with Fc comprising P329G, L234A and L235A with 1:1 stoichiometry. The epitope is disclosed to include positions N325 to P331 (including G329), and also S267 to E272.

In some embodiments, the antigen-binding moiety comprises the CDRs, FRs and/or the VH and/or VL regions of an antigen-binding molecule described herein that binds to a variant Fc domain according to the present disclosure, or comprises CDRs, FRs and/or VH and/or VL regions which are derived from those of an antigen-binding molecule described herein that binds to a variant Fc domain according to the present disclosure. In some embodiments, an antigen-binding molecule that binds to a variant Fc domain according to the present disclosure is selected from: αP329G_VH1/VL1, αP329G_VH2/VL1, and αP329G_VH3/VL1.

In some embodiments, the antigen-binding moiety comprises a VH region according to (1) or (2) below:
(1) a VH region incorporating the following CDRs:
   HC-CDR1 having the amino acid sequence of SEQ ID NO: 11
   HC-CDR2 having the amino acid sequence of SEQ ID NO: 12
   HC-CDR3 having the amino acid sequence of SEQ ID NO: 13,
or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.
(2) a VH region incorporating the following CDRs:
   HC-CDR1 having the amino acid sequence of SEQ ID NO: 11
   HC-CDR2 having the amino acid sequence of SEQ ID NO:19
   HC-CDR3 having the amino acid sequence of SEQ ID NO:13,
or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.

In some embodiments, the antigen-binding moiety comprises a VH region according to (3) or (4) below:
(3) a VH region incorporating the following FRs:
   HC-FR1 having the amino acid sequence of SEQ ID NO:14
   HC-FR2 having the amino acid sequence of SEQ ID NO:15
   HC-FR3 having the amino acid sequence of SEQ ID NO:16
   HC-FR4 having the amino acid sequence of SEQ ID NO:17,
   or a variant thereof in which 1 or 2 or 3 amino acids in HC-FRI, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.
(4) a VH region incorporating the following FRs:
   HC-FR1 having the amino acid sequence of SEQ ID NO:21
   HC-FR2 having the amino acid sequence of SEQ ID NO:15
   HC-FR3 having the amino acid sequence of SEQ ID NO:22
   HC-FR4 having the amino acid sequence of SEQ ID NO:17,
   or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.

In some embodiments, the antigen-binding moiety comprises a VH region comprising the CDRs according to (1) or (2) above, and the FRs according to (3) or (4) above.

In some embodiments, the antigen-binding moiety comprises a VH region according to (5) or (6) below:
(5) a VH region comprising the CDRs according to (1) and the FRs according to (3).
(6) a VH region comprising the CDRs according to (2) and the FRs according to (4).
(7) a VH region comprising the CDRs according to (2) and the FRs according to (3).

In some embodiments, the antigen-binding moiety comprises a VH region according to one of (8) to (10) below:
(8) a VH region comprising an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%, sequence identity, to the amino acid sequence of SEQ ID NO:10.
(9) a VH region comprising an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%, sequence identity, to the amino acid sequence of SEQ ID NO:18.
(10) a VH region comprising an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%, sequence identity, to the amino acid sequence of SEQ ID NO:20.

In some embodiments, the antigen-binding moiety comprises a VL region according to (11) below:
(11) a VL region incorporating the following CDRs:
   LC-CDR1 having the amino acid sequence of SEQ ID NO:24
   LC-CDR2 having the amino acid sequence of SEQ ID NO:25
   LC-CDR3 having the amino acid sequence of SEQ ID NO:26.
or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.

In some embodiments, the antigen-binding moiety comprises a VL region according to (12) below:
(12) a VL region incorporating the following FRs:
   LC-FRI having the amino acid sequence of SEQ ID NO:27
   LC-FR2 having the amino acid sequence of SEQ ID NO:28
   LC-FR3 having the amino acid sequence of SEQ ID NO:29
   LC-FR4 having the amino acid sequence of SEQ ID NO:30,
   or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.

In some embodiments, the antigen-binding moiety comprises a VL region according to (13) below:
(13) a VL region comprising the CDRs according to (11) and the FRs according to (12).

In some embodiments, the antigen-binding moiety comprises a VL region according to (14) below:
(14) a VL region comprising an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%, sequence identity, to the amino acid sequence of SEQ ID NO:23.

In some embodiments, the antigen-binding moiety comprises a VH region according to any one of (1) to (10) above, and a VL region according to any one of (11) to (14) above.

In some embodiments, a component of an antigen-binding moiety comprises or consists of a polypeptide or polypeptides comprising a VH region comprising HC-CDR1, HC-CDR2 and HC-CDR3 as indicated in column A of Table A. In some embodiments, a component of an antigen-binding moiety comprises or consists of a polypeptide or polypeptides comprising a VL region comprising LC-CDR1, LC-CDR2 and LC-CDR3 as indicated in column B of Table A.

In some embodiments, a component of an antigen-binding moiety comprises or consists of a polypeptide or polypeptides comprising a VH region comprising HC-FR1, HC-FR2, HC-FR3 and HC-FR4 as indicated in column A of Table B. In some embodiments, a component of an antigen-binding moiety comprises or consists of a polypeptide or polypeptides comprising a VL region comprising LC-FR1, LC-FR2, LC-FR3, and LC-FR4 as indicated in column B of Table B.

In some embodiments, a component of an antigen-binding moiety comprises or consists of a polypeptide or polypeptides comprising an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to an amino acid sequence indicated in column A of Table C. In some embodiments, a component of an antigen-binding moiety comprises or consists of a polypeptide or polypeptides comprising an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to an amino acid sequence indicated in column B of Table C.

In some embodiments, a component of an antigen-binding moiety comprises or consists of an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:10, 18 or 20. In some embodiments, a component of an antigen-binding moiety comprises or consists of an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:23.

It will be appreciated that where components of an antigen-binding moiety are provided in aspects and embodiments of the present disclosure, it is intended that the components provided are complementary, and capable of associating to form the (complete, functional) antigen-binding moiety.

Substitutions of amino acids in accordance with the present disclosure may be biochemically conservative. In some embodiments, where an amino acid to be substituted is provided in one of rows 1 to 5 of the table below, the replacement amino acid of the substitution is another, non-identical amino acid provided in the same row:

| Row | Shared property | Amino acids |
|---|---|---|
| 1 | Hydrophobic | Met, Ala, Val, Leu, Ile, Trp, Tyr, |
| | | Phe, Norleucine |
| 2 | Neutral hydrophilic | Cys, Ser, Thr, Asn, Gln |
| 3 | Acidic or negatively-charged | Asp, Glu |
| 4 | Basic or positively-charged | His, Lys, Arg |
| 5 | Orientation influencing | Gly, Pro |

By way of illustration, in some embodiments wherein substitution is of a Met residue, the replacement amino acid may be selected from Ala, Val, Leu, Ile, Trp, Tyr, Phe and Norleucine.

In some embodiments, a replacement amino acid in a substitution may have the same side chain polarity as the amino acid residue it replaces. In some embodiments, a replacement amino acid in a substitution may have the same side chain charge (at pH 7.4) as the amino acid residue it replaces:

| | | Side-chain charge |
|---|---|---|
| Amino Acid | Side-chain polarity | (pH 7.4) |
| Alanine | nonpolar | neutral |
| Arginine | basic polar | positive |
| Asparagine | polar | neutral |
| Aspartic acid | acidic polar | negative |
| Cysteine | nonpolar | neutral |
| Glutamic acid | acidic polar | negative |
| Glutamine | polar | neutral |
| Glycine | nonpolar | neutral |
| Histidine | basic polar | positive (10%) |
| | | neutral (90%) |
| Isoleucine | nonpolar | neutral |
| Leucine | nonpolar | neutral |
| Lysine | basic polar | positive |
| Methionine | nonpolar | neutral |
| Phenylalanine | nonpolar | neutral |
| Proline | nonpolar | neutral |
| Serine | polar | neutral |
| Threonine | polar | neutral |
| Tryptophan | nonpolar | neutral |
| Tyrosine | polar | neutral |
| Valine | nonpolar | neutral |

That is, in some embodiments, a nonpolar amino acid is substituted with another, non-identical nonpolar amino acid. In some embodiments, a polar amino acid is substituted with another, non-identical polar amino acid. In some embodiments, an acidic polar amino acid is substituted with another, non-identical acidic polar amino acid. In some embodiments, a basic polar amino acid is substituted with another, non-identical basic polar amino acid. In some embodiments, a neutral amino acid is substituted with another, non-identical neutral amino acid. In some embodiments, a positive amino acid is substituted with another, non-identical positive amino acid. In some embodiments, a negative amino acid is substituted with another, non-identical negative amino acid.

In some embodiments, substitution(s) may be functionally conservative. That is, in some embodiments, the substitution may not affect (or may not substantially affect) one or more functional properties (e.g. target antigen binding) of the antigen-binding moiety comprising the substitution, as compared to the equivalent unsubstituted molecule.

In some embodiments, an antigen-binding moiety of the present disclosure comprises a VH as described herein. In some embodiments, an antigen-binding moiety comprises a VL as described herein. In some embodiments, an antigen-binding moiety comprises one or more antibody heavy chain constant regions (CH). In some embodiments, an antigen-binding moiety comprises one or more antibody light chain constant regions (CL). In some embodiments, an antigen-binding moiety comprises a CH1, CH2 region and/or a CH3 region of an immunoglobulin (Ig). In some embodiments, an antigen-binding moiety comprises a linker sequence as described herein.

In some embodiments, the antigen-binding moiety of the present disclosure comprises a polypeptide or polypeptides comprising: (i) a VH region comprising HC-CDR1, HC-CDR2 and HC-CDR3 as indicated in column A of Table A, and (ii) a VL region comprising LC-CDR1, LC-CDR2 and LC-CDR3 as indicated in column B of Table A, wherein the sequences of columns A and B are selected from the same row of Table A.

In some embodiments, the antigen-binding moiety of the present disclosure comprises a polypeptide or polypeptides comprising: (i) a VH region comprising HC-CDR1 according to SEQ ID NO:11, HC-CDR2 according to SEQ ID NO: 19, and HC-CDR3 according to SEQ ID NO: 13, and (ii) a VL region comprising LC-CDR1 according to SEQ ID NO:24, LC-CDR2 according to SEQ ID NO:25, and LC-CDR3 according to SEQ ID NO:26.

In some embodiments, the antigen-binding moiety of the present disclosure comprises a polypeptide or polypeptides comprising: (i) a VH region comprising HC-FR1, HC-FR2, HC-FR3 and HC-FR4 as indicated in column A of Table B, and (ii) a VL region comprising LC-FR1, LC-FR2, LC-FR3, and LC-FR4 as indicated in column B of Table B, wherein the sequences of columns A and B are selected from the same row of Table B.

In some embodiments, the antigen-binding moiety of the present disclosure comprises a polypeptide or polypeptides comprising: (i) a VH region comprising HC-FR1 according to SEQ ID NO:21, HC-FR2 according to SEQ ID NO: 15, HC-FR3 according to SEQ ID NO:22, and HC-FR4 according to SEQ ID NO: 17, and (ii) a VL region comprising LC-FRI according to SEQ ID NO:27. LC-FR2 according to SEQ ID NO: 28, LC-FR3 according to SEQ ID NO:29, and LC-FR4 according to SEQ ID NO:30.

In some embodiments, the antigen-binding moiety of the present disclosure comprises a polypeptide or polypeptides comprising: (i) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to an amino acid sequence indicated in column A of Table C, and (ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to an amino acid sequence indicated in column B of Table C, wherein the sequences of columns A and B are selected from the same row of Table C.

In some embodiments, an antigen-binding moiety of the present disclosure comprises an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:10, 18 or 20. In some embodiments, an antigen-binding moiety of the present disclosure comprises an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:23.

In some embodiments, an antigen-binding moiety of the present disclosure comprises an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:20. In some embodiments, an antigen-binding moiety of the present disclosure comprises an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:23.

In some embodiments, an antigen-binding moiety of the present disclosure comprises, or consists of, an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:33. In some embodiments, an antigen-binding moiety comprises, or consists of, an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:34. In preferred embodiments, an antigen-binding moiety comprises, or consists of, an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:35.

### Chimeric Antigen Receptors (CARs)

In one particular embodiment, the membrane-anchored antigen binding (MAB) polypeptide is a chimeric antigen receptor (CAR).

The term “chimeric antigen receptor” or “chimeric receptor” or “CAR” refers to an antigen binding receptor constituted of an extracellular portion of an antigen binding moiety (e.g. a single chain antibody domain) fused by a transmembrane domain and optionally further spacer sequence(s) to intracellular signaling/co-signalling domains (such as e.g. of CD3z and CD28).

In some aspects, the transmembrane domain comprises part of a murine/mouse or preferably of a human transmembrane domain. An example for such a transmembrane domain is a transmembrane domain of CD8, for example, having the amino acid sequence as shown herein in SEQ ID NO: 73. In the context of the present invention, the transmembrane domain of the CAR may comprise/consist of an amino acid sequence as shown in SEQ ID NO: 73.

In another embodiment, the herein provided CAR may comprise the transmembrane domain of CD28 which is located at amino acids 153 to 179, 154 to 179, 155 to 179, 156 to 179, 157 to 179, 158 to 179, 159 to 179, 160 to 179, 161 to 179, 162 to 179, 163 to 179, 164 to 179, 165 to 179, 166 to 179, 167 to 179, 168 to 179, 169 to 179, 170 to 179, 171 to 179, 172 to 179, 173 to 179, 174 to 179, 175 to 179, 176 to 179, 177 to 179 or 178 to 179 of the human full length CD28 protein as shown in SEQ ID NO:86 (as encoded by the cDNA shown in SEQ ID NO:85).

Alternatively, any protein having a transmembrane domain, as provided among others by the CD nomenclature, may be used as a transmembrane domain of the CAR protein used according to the invention.

In some embodiments, the transmembrane domain comprises the transmembrane domain of any one of the group consisting of CD27 (SEQ ID NO:81 as encoded by SEQ ID NO:83), CD137 (SEQ ID NO:92 as encoded by SEQ ID NO:91), OX40 (SEQ ID NO:96, as encoded by SEQ ID NO:95), ICOS (SEQ ID NO: 100 as encoded by SEQ ID NO:99), DAP10 (SEQ ID NO: 104 as encoded by SEQ ID NO:103), DAP12 (SEQ ID NO: 108 as encoded by SEQ ID NO: 107), CD3z (SEQ ID NO: 113 as encoded by SEQ ID NO: 114). FCGR3A (SEQ ID NO: 115 as encoded by SEQ ID NO:116). NKG2D (SEQ ID NO:119 as encoded by SEQ ID NO: 120), CD8 (SEQ ID NO: 129 as encoded by SEQ ID NO:130), CD40 (SEQ ID NO: 133 as encoded by SEQ ID NO: 134) or a fragment of the transmembrane thereof that retains the capability to confine the CAR to the membrane.

Human sequences might be beneficial, for example because (parts) of the transmembrane domain might be accessible from the extracellular space and hence to the immune system of a patient. In a preferred embodiment, the transmembrane domain comprises a human sequence. In such embodiments, the transmembrane domain comprises the transmembrane domain of any one of the group consisting of human CD27 (SEQ ID NO:82 as encoded by SEQ ID NO:81), human CD137 (SEQ ID NO:90 as encoded by SEQ ID NO:89), human OX40) (SEQ ID NO:94, as encoded by SEQ ID NO:93), human ICOS (SEQ ID NO:98 as encoded by SEQ ID NO:917), human DAP10 (SEQ ID NO: 103 as encoded by SEQ ID NO: 102), human DAP12 (SEQ ID NO:106 as encoded by SEQ ID NO: 105), human CD3z (SEQ ID NO: 111 as encoded by SEQ ID NO: 110), human FCGR3A (SEQ ID NO: 113 as encoded by SEQ ID NO: 114), human NKG2D (SEQ ID NO: 117 as encoded by SEQ ID NO:118), human CD8 (SEQ ID NO: 127 as encoded by SEQ ID NO: 128), human CD40 (SEQ ID NO:131 as encoded by SEQ ID NO: 132) or a fragment of the transmembrane thereof that retains the capability to anchor the CAR to the membrane.

Preferably, the CAR used according to the present invention comprises at least one stimulatory signaling domain and/or at least one co-stimulatory signaling domain. Accordingly, the herein provided CAR preferably comprises a stimulatory signaling domain, which provides T cell activation. The herein provided CAR may comprise a stimulatory signaling domain which is a fragment/polypeptide part of murine/mouse or human CD3z (the UniProt Entry of the human CD3z is P20963 (version number 177 with sequence number 2: the UniProt Entry of the murine/mouse CD3z is P24161 (primary citable accession number) or Q9D3G3 (secondary citable accession number) with the version number 143 and the sequence number 1)). FCGR3A (the UniProt Entry of the human FCGR3A is P08637 (version number 178 with sequence number 2)), or NKG2D (the UniProt Entry of the human NKG2D is P26718 (version number 151 with sequence number 1): the UniProt Entry of the murine/mouse NKG2D is 054709 (version number 132 with sequence number 2)).

Thus, the stimulatory signaling domain which is comprised in the herein provided CAR may be a fragment/polypeptide part of the full length of CD3z. FCGR3A or NKG2D. The amino acid sequences of the murine/mouse full length of CD3z, or NKG2D are shown herein as SEQ ID NOs: 111 (CD3z), 115 (FCGR3A) or 119 (NKG2D) (murine/mouse as encoded by the DNA sequences shown in SEQ ID NOs: 112 (CD3z), 116 (FCGR3A) or 120 (NKG2D). The amino acid sequences of the human full length CD3z, FCGR3A or NKG2D are shown herein as SEQ ID NOs: 109 (CD3z), 113 (FCGR3A) or 117 (NKG2D) (human as encoded by the DNA sequences shown in SEQ ID NOs: 110 (CD3z), 114 (FCGR3A) or 118 (NKG2D)). The CAR used according to the present invention may comprise fragments of CD3z. FCGR3A or NKG2D as stimulatory domain, provided that at least one signaling domain is comprised. In particular, any part/fragment of CD3z, FCGR3A, or NKG2D is suitable as stimulatory domain as long as at least one signaling motive is comprised. However, more preferably, the CAR used according to the present invention comprises polypeptides which are derived from human origin. Thus, more preferably, the herein provided CAR comprises the amino acid sequences as shown herein as SEQ ID NOs: 109 (CD3z), 113 (FCGR3A) or 117 (NKG2D) (human as encoded by the DNA sequences shown in SEQ ID NOs: 110 (CD3z), 114 (FCGR3A) or 118 (NKG2D)). In one embodiment, the CAR used according to the present invention may comprise or consist of the amino acid sequence shown in SEQ ID NO: 146, SEQ ID NO: 149, SEQ ID NO: 151, SEQ ID NO: 154. In further embodiments the CAR comprises the sequence as shown in SEQ ID NO: 151 or a sequence which has up to 1, 2, 3, 4, 5, 6, 7, 8, 9, 10, 11, 12, 13, 14, 15, 16, 17, 18, 19, 20, 21, 22, 23, 23, 24, 25, 26, 27, 28, 29 or 30 substitutions, deletions or insertions in comparison to SEQ ID NO:151 and which is characterized by having a stimulatory signaling activity. Specific configurations of CARs comprising a stimulatory signaling domain (SSD) are provided herein below and in the Examples and Figures. The stimulatory signaling activity can be determined; e.g., by enhanced cytokine release, as measured by ELISA (IL-2, IFNγ, TNFα), enhanced proliferative activity (as measured by enhanced cell numbers), or enhanced lytic activity as measured by LDH release assays.

Furthermore, the herein provided CAR preferably comprises at least one co-stimulatory signaling domain which provides additional activity to the T cell. The herein provided CAR may comprise a co-stimulatory signaling domain which is a fragment/polypeptide part of murine/mouse or human CD28 (the UniProt Entry of the human CD28 is P10747 (version number 173 with sequence number 1); the UniProt Entry of the murine/mouse CD28 is P31041 (version number 134 with sequence number 2)), CD137 (the UniProt Entry of the human CD137 is Q07011 (version number 145 with sequence number 1); the UniProt Entry of murine/mouse CD137 is P20334 (version number 139 with sequence number 1)), OX40) (the UniProt Entry of the human OX40 is P23510 (version number 138 with sequence number 1); the UniProt Entry of murine/mouse OX40 is P43488 (version number 119 with sequence number 1)), ICOS (the UniProt Entry of the human ICOS is Q9Y6W8 (version number 126 with sequence number 1)): the UniProt Entry of the murine/mouse ICOS is Q9WV40 (primary citable accession number) or Q9JL17 (secondary citable accession number) with the version number 102 and sequence version 2)), CD27 (the UniProt Entry of the human CD27 is P26842 (version number 160 with sequence number 2); the Uniprot Entry of the murine/mouse CD27 is P41272 (version number 137 with sequence version 1)), 4-1-BB (the UniProt Entry of the murine/mouse 4-1-BB is P20334 (version number 140 with sequence version 1); the UniProt Entry of the human 4-1-BB is Q07011 (version number 146 with sequence version)), DAP10 (the UniProt Entry of the human DAP10 is Q9UBJ5 (version number 25 with sequence number 1): the UniProt entry of the murine/mouse DAP10 is Q9QUJ0 (primary citable accession number) or Q9R1E7 (secondary citable accession number) with the version number 101 and the sequence number 1)) or DAP12 (the UniProt Entry of the human DAP12 is 043914 (version number 146 and the sequence number 1); the UniProt entry of the murine/mouse DAP12 is 0054885 (primary citable accession number) or Q9RIE7 (secondary citable accession number) with the version number 123 and the sequence number 1). The herein provided CAR may comprise a co-stimulatory signaling domain which is a fragment/polypeptide part of human or murine/mouse CD40 (SEQ ID NOs: 131, 133,) In certain embodiments of the present invention the CAR may comprise one or more, i.e. 1, 2, 3, 4, 5, 6 or 7 of the herein defined co-stimulatory signaling domains. Accordingly, in the context of the present invention, the CAR may comprise a fragment/polypeptide part of a murine/mouse or preferably of a human CD137 as first co-stimulatory signaling domain and the second co-stimulatory signaling domain is selected from the group consisting of the murine/mouse or preferably of the human CD27, CD28, CD137, OX40, ICOS, DAP10 and DAP12, or fragments thereof. Preferably, the CAR comprises a co-stimulatory signaling domain which is derived from a human origin. Thus, more preferably, the co-stimulatory signaling domain(s) which is (are) comprised in the CAR used according to the present invention may comprise or consist of the amino acid sequence as shown in SEQ ID NO: 74 or 76.

Thus, the co-stimulatory signaling domain which may be comprised in the herein provided CAR is a fragment/polypeptide part of the full length CD27, CD28, CD137, OX40, ICOS, DAP10, DAP12 or CD40. The amino acid sequences of the murine/mouse full length CD27, CD28, CD137, OX40, ICOS, CD27, DAP10, DAP12 and CD40 are shown herein as SEQ ID NOs: 82 (CD27), 89 (CD28), 93 (CD137), 97 (OX40), 101 (ICOS), 105 (DAP10), 109 (DAP12), 133 (CD40) (murine/mouse as encoded by the DNA sequences shown in SEQ ID NOs: 83 (CD27), 87 (CD28), 91 (CD137), 95 (OX40), 99 (ICOS), 103 (DAP10), 107 (DAP12), 134 (CD40))). However, because human sequences are most preferred in the context of the present invention, the co-stimulatory signaling domain which may be optionally comprised in the herein provided CAR protein is a fragment/polypeptide part of the human full length CD27, CD28, CD137, OX40, ICOS, DAP10, DAP12 or CD40. The amino acid sequences of the human full length CD27, CD28, CD137, OX40, ICOS, DAP10, DAP12 or CD40) are shown herein as SEQ ID NOs: 82, (CD27), 86 (CD28), 90 (CD137), 94 (OX40), 98 (ICOS), 102 (DAP10), 106 (DAP12), 131 (CD40) (human as encoded by the DNA sequences shown in SEQ ID NOs: 81 (CD27), 85 (CD28), 89 (CD137), 93 (OX40), 97 (ICOS), 101 (DAP10), 105 (DAP12), 132 (CD40)).

In one preferred embodiment, the CAR comprises CD28 or a fragment thereof capable of T cell activation as co-stimulatory signaling domain. The herein provided CAR may comprise a fragment of CD28 as co-stimulatory signaling domain, provided that at least one signaling domain of CD28 is comprised. In particular, any part/fragment of CD28 is suitable for the CAR used according to the invention as long as at least one of the signaling motives of CD28 is comprised. The co-stimulatory signaling domains PYAP (AA 208 to 211 of CD28) and YMNM (AA 191 to 194 of CD28) are beneficial for the function of the CD28 polypeptide and the functional effects enumerated above. The amino acid sequence of the YMNM domain is shown in SEQ ID NO:97: the amino acid sequence of the PYAP domain is shown in SEQ ID NO: 98. Accordingly, in the CAR used according to the present invention, the CD28 polypeptide preferably comprises a sequence derived from intracellular domain of a CD28 polypeptide having the sequences YMNM (SEQ ID NO:121) and/or PYAP (SEQ ID NO:122). In other embodiments, one or both of these domains are mutated to FMNM (SEQ ID NO: 123) and/or AYAA (SEQ ID NO:124), respectively. Either of these mutations reduces the ability of a transduced cell comprising the CAR to release cytokines without affecting its ability to proliferate and can advantageously be used to prolong the viability and thus the therapeutic potential of the transduced cells. Or, in other words, such a non-functional mutation preferably enhances the persistence of the cells which are transduced with the herein provided CAR in vivo. These signaling motives may, however, be present at any site within the intracellular domain of the herein provided CAR.

In another preferred embodiment, the CAR comprises CD137 or a fragment thereof capable of T cell activation as co-stimulatory signaling domain. The herein provided CAR may comprise a fragment of CD137 as co-stimulatory signaling domain, provided that at least one signaling domain of CD137 is comprised. In particular, any part/fragment of CD137 is suitable for the CAR used according to the invention as long as at least one of the signaling motives of CD137 is comprised. In a preferred embodiment, the CD137 polypeptide which is comprised in the CAR protein used according to the present invention comprises or consists of the amino acid sequence shown in SEQ ID NO:76.

Specific configurations of CARs comprising a co-stimulatory signaling domain (CSD) are provided herein below and in the Examples and Figures. The co-stimulatory signaling activity can be determined; e.g., by enhanced cytokine release, as measured by ELISA (IL-2, IFNγ, TNFα), enhanced proliferative activity (as measured by enhanced cell numbers), or enhanced lytic activity as measured by LDH release assays. As mentioned above, in an embodiment of the present invention, the co-stimulatory signaling domain of the CAR may be derived from human CD28 and/or CD137 gene or fragments there capable of T cell activation, defined as cytokine production, proliferation and lytic activity of the T cell. CD28 and/or CD137 activity can be measured by release of cytokines by ELISA or flow cytometry of cytokines such as interferon-gamma (IFN-γ) or interleukin 2 (IL-2), proliferation of T cells measured e.g. by ki67-measurement, cell quantification by flow cytometry, or lytic activity as assessed by real time impedence measurement of the target cell (by using e.g. an ICELLligence instrument as described e.g. in Thakur et al., Biosens Bioelectron. 35 (1) (2012), 503-506; Krutzik et al., Methods Mol Biol. 699 (2011), 179-202; Ekkens et al., Infect Immun. 75 (5) (2007), 2291-2296; Ge et al., Proc Natl Acad Sci USA. 99 (5) (2002), 2983-2988; Düwell et al., Cell Death Differ. 21 (12) (2014), 1825-1837. Erratum in: Cell Death Differ. 21 (12) (2014), 161).

The herein provided CAR may comprise at least one linker (or “spacer”). A linker is usually a peptide having a length of up to 20 amino acids. Accordingly, in the context of the present invention the linker may have a length of 1, 2, 3, 4, 5, 6, 7, 8, 9, 10, 11, 12, 13, 14, 15, 16, 17, 18, 19 or 20 amino acids. For example, the herein provided CAR may comprise a linker between the extracellular domain comprising at least one antigen binding moiety capable of specific binding to a mutated Fc domain, the transmembrane domain, the co-stimulatory signaling domain and/or the stimulatory signaling domain. Furthermore, the herein provided CAR may comprise a linker in the antigen binding moiety, in particular between immunoglobulin domains of the antigen binding moiety (such as between VH and VL domains of a scFv). Such linkers have the advantage that they increase the probability that the different polypeptides of the CAR (i.e. the extracellular domain comprising at least one antigen binding moiety, the transmembrane domain, the co-stimulatory signaling domain and/or the stimulatory signaling domain) fold independently and behave as expected. Thus, in the context of the present invention, the extracellular domain comprising at least one antigen binding moiety, the transmembrane domain, the co-stimulatory signaling domain and the stimulatory signaling domain may be comprised in a single-chain multi-functional polypeptide. A single-chain fusion construct e.g. may consist of (a) polypeptide(s) comprising (an) extracellular domain(s) comprising at least one antigen binding moiety, (an) transmembrane domain(s), (a) co-stimulatory signaling domain(s) and/or (a) stimulatory signaling domain(s). Accordingly, the antigen binding moiety, the transmembrane domain, the co-stimulatory signaling domain and the stimulatory signaling domain may be connected by one or more identical or different peptide linker as described herein. For example, in the herein provided CAR the linker between the extracellular domain comprising at least one antigen binding moiety and the transmembrane domain may comprise or consist of the amino and amino acid sequence as shown in SEQ ID NO: 78. In another embodiment, the linker between the antigen binding moiety and the transmembrane domain comprises or consists of the amino and amino acid sequence as shown in SEQ ID NO:80. Accordingly, the transmembrane domain, the co-stimulatory signaling domain and/or the stimulatory domain may be connected to each other by peptide linkers or alternatively, by direct fusion of the domains.

In preferred embodiments according to the invention the antigen binding moiety is a single-chain variable fragment (scFv). A scFv is a fusion protein of the variable domains of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. In a preferred embodiment, the linker connects the N-terminus of the VL domain with the C-terminus of the VH domain. For example, in the herein provided CAR the linker may have the amino and amino acid sequence as shown in SEQ ID NO: 77, scFv antibodies are, e.g. described in Houston, J. S., Methods in Enzymol. 203 (1991) 46-96).

In some embodiments according to the invention the antigen binding moiety is a single chain Fab fragment or scFab which is a polypeptide consisting of an heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain.

The herein provided CAR or parts thereof may comprise a signal peptide. Such a signal peptide will bring the protein to the surface of the T cell membrane. For example, in the herein provided CAR the signal peptide may have the amino and amino acid sequence as shown in SEQ ID NO:125 (as encoded by the DNA sequence shown in SEQ ID NO:126).

The components of the CARs as described herein can be fused to each other in a variety of configurations to generate T cell activating CARs.

In some embodiments, the CAR comprises an extracellular domain composed of a heavy chain variable domain (VH) and a light chain variable domain (VL) connected to a transmembrane domain. In preferred embodiments, the VH domain is fused at the C-terminus to the N-terminus of the VL domain, optionally through a peptide linker. In other embodiments, the CAR further comprises a stimulatory signaling domain and/or a co-stimulatory signaling domain. In a specific such embodiment, the CAR essentially consists of a VH domain and a VL domain, a transmembrane domain, and optionally a stimulatory signaling domain connected by one or more peptide linkers, wherein the VH domain is fused at the C-terminus to the N-terminus of the VL domain, and the VL domain is fused at the C-terminus to the N-terminus of the transmembrane domain, wherein the transmembrane domain is fused at the C-terminus to the N-terminus of the stimulatory signaling domain. Optionally, the CAR further comprises a co-stimulatory signaling domain. In one such specific embodiment, the CAR essentially consists of a VH domain and a VL domain, a transmembrane domain, a stimulatory signaling domain and a co-stimulatory signaling domain connected by one or more peptide linkers, wherein the VH domain is fused at the C-terminus to the N-terminus of the VL domain, and the VL domain is fused at the C-terminus to the N-terminus of the transmembrane domain, wherein the transmembrane domain is fused at the C-terminus to the N-terminus of the stimulatory signaling domain, wherein the stimulatory signaling domain is fused at the C-terminus to the N-terminus of the co-stimulatory signaling domain. In an alternative embodiment, the co-stimulatory signaling domain is connected to the transmembrane domain instead of the stimulatory signaling domain. In a preferred embodiment, the CAR essentially consists of a VH domain and a VL domain, a transmembrane domain, a co-stimulatory signaling domain and a stimulatory signaling domain connected by one or more peptide linkers, wherein the VH domain is fused at the C-terminus to the N-terminus of the VL domain, and the VL domain is fused at the C-terminus to the N-terminus of the transmembrane domain, wherein the transmembrane domain is fused at the C-terminus to the N-terminus of the co-stimulatory signaling domain, wherein the co-stimulatory signaling domain is fused at the C-terminus to the N-terminus of the stimulatory signaling domain.

The antigen binding moiety, the transmembrane domain and the stimulatory signaling and/or co-stimulatory signaling domains may be fused to each other directly or through one or more peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ or G₄(SG₄)ₙ peptide linkers, wherein “n” is generally a number between 1 and 10, typically between 2 and 4. A preferred peptide linker for connecting the antigen binding moiety and the transmembrane moiety is GGGGS (G₄S) according to SEQ ID NO 78. Another preferred peptide linker for connecting the antigen binding moiety and the transmembrane moiety is KPTTTPAPRPPTPAPTIASQPLSLRPEACRPAAGGAVHTRGLDFACD (CD8stalk) according to SEQ ID NO 80. An exemplary peptide linker suitable for connecting variable heavy chain domain (VH) and the variable light chain domain (VL) is GGGSGGGSGGGSGGGS (GAS), according to SEQ ID NO 77.

Additionally, linkers may comprise (a portion of) an immunoglobulin hinge region. Particularly where an antigen binding moiety is fused to the N-terminus of a transmembrane domain, it may be fused via an immunoglobulin hinge region or a portion thereof, with or without an additional peptide linker.

As described herein, the CARs used according to the present invention comprise an extracellular domain comprising at least one antigen binding moiety. A CAR with a single antigen binding moiety capable of specific binding to a target cell antigen is useful and preferred, particularly in cases where high expression of the CAR is needed. In such cases, the presence of more than one antigen binding moiety specific for the target cell antigen may limit the expression efficiency of the CAR. In other cases, however, it will be advantageous to have an CAR comprising two or more antigen binding moieties specific for a target cell antigen, for example to optimize targeting to the target site or to allow crosslinking of target cell antigens.

In one particular embodiment, the CAR comprises one antigen binding moiety capable of specific binding to a mutated Fc domain, in particular a variant CH2-CH3 region comprising the amino acid substitution P329G according to EU numbering. In one embodiment, the antigen binding moiety capable of specific binding to a variant CH2-CH3 region comprising the amino acid substitution P329G according to EU numbering is a scFv.

In one embodiment, the antigen binding moiety is fused at the C-terminus of the scFv fragment to the N-terminus of a transmembrane domain, optionally through a peptide linker. In one embodiment the peptide linker comprises the amino acid sequence KPTTTPAPRPPTPAPTIASQPLSLRPEACRPAAGGAVHTRGLDFACD (SEQ ID NO:80). In one embodiment, the transmembrane domain is a transmembrane domain selected from the group consisting of the CD8, the CD4, the CD3z, the CD40, the FCGR3A, the NKG2D, the CD27, the CD28, the CD137, the OX40, the ICOS, the DAP10 or the DAP12 transmembrane domain or a fragment thereof. In a preferred embodiment, the transmembrane domain is the CD8 transmembrane domain or a fragment thereof. In a particular embodiment, the transmembrane domain comprises or consists of the amino acid sequence of IYIWAPLAGTCGVLLLSLVIT (SEQ ID NO:73). In one embodiment, the CAR further comprises a co-stimulatory signaling domain (CSD). In one embodiment, the transmembrane domain of the CAR is fused at the C-terminus to the N-terminus of a co-stimulatory signaling domain. In one embodiment, the co-stimulatory signaling domain is individually selected from the group consisting of the intracellular domain of CD27, of CD28, of CD137, of OX40), of ICOS, of DAP10 and of DAP12, or fragments thereof as described herein before. In a preferred embodiment, the co-stimulatory signaling domain is the intracellular domain of CD28 or a fragment thereof. In one preferred embodiment, the co-stimulatory signaling domain comprises the intracellular domain of CD28 or a fragment thereof that retains CD28 signaling. In another preferred embodiment, the co-stimulatory signaling domain comprises the intracellular domain of CD137 or a fragment thereof that retains CD137 signaling. In a particular embodiment the co-stimulatory signaling domain comprises or consists of SEQ ID NO:74. In another particular embodiment the co-stimulatory signaling domain comprises or consists of SEQ ID NO:76. In one embodiment, the CAR further comprises a stimulatory signaling domain. In one embodiment, the co-stimulatory signaling domain of the CAR is fused at the C-terminus to the N-terminus of the stimulatory signaling domain. In one embodiment, the at least one stimulatory signaling domain is individually selected from the group consisting of the intracellular domain of CD3z. FCGR3A and NKG2D, or fragments thereof. In a preferred embodiment, the co-stimulatory signaling domain is the intracellular domain of CD3z or a fragment thereof that retains CD3z signaling. In a particular embodiment the co-stimulatory signaling domain comprises or consists of SEQ ID NO:75.

In one embodiment, the CAR is fused to a reporter protein, particularly to GFP or enhanced analogs thereof. In one embodiment, the CAR is fused at the C-terminus to the N-terminus of eGFP (enhanced green fluorescent protein), optionally through a peptide linker as described herein. In a preferred embodiment, the peptide linker is GEGRGSLLTCGDVEENPGP (T2A) according to SEQ ID NO:79.

In a particular embodiment, the CAR comprises a transmembrane domain and an extracellular domain comprising at least one antigen binding moiety, wherein the at least one antigen binding moiety is a scFv capable of specific binding to a variant CH2-CH3 region comprising the amino acid substitution P329G according to EU numbering. The P329G mutation reduces Fcγ receptor binding. In one embodiment, the CAR comprises a transmembrane domain (TD), a co-stimulatory signaling domain (CSD) and a stimulatory signaling domain (SSD). In one such embodiment, the CAR has the configuration scFv-TD-CSD-SSD. In a preferred embodiment, the CAR has the configuration VH-VL-TD-CSD-SSD. In a more specific such embodiment, the CAR has the configuration VH-linker-VL-linker-TD-CSD-SSD.

In a particular embodiment, the antigen binding moiety is a scFv capable of specific binding to a variant CH2-CH3 region comprising the amino acid substitution P329G according to EU numbering, wherein the antigen binding moiety comprises at least one heavy chain complementarity determining region (CDR) selected from the group consisting of SEQ ID NO:11, SEQ ID NO: 12 and SEQ ID NO:13 and at least one light chain CDR selected from the group of SEQ ID NO:24, SEQ ID NO:25, SEQ ID NO:26.

In another particular embodiment, the antigen binding moiety is a scFv capable of specific binding to a variant CH2-CH3 region comprising the amino acid substitution P329G according to EU numbering, wherein the antigen binding moiety comprises at least one heavy chain complementarity determining region (CDR) selected from the group consisting of SEQ ID NO: 11, SEQ ID NO: 19 and SEQ ID NO:13 and at least one light chain CDR selected from the group of SEQ ID NO:24, SEQ ID NO:25, SEQ ID NO: 26.

In one embodiment, provided is a CAR capable of specific binding to a variant CH2-CH3 region comprising the amino acid substitution P329G according to EU numbering, wherein the CAR comprises in order from the N-terminus to the C-terminus:
(i) a heavy chain variable domain (VH) comprising the heavy chain complementarity determining region (CDR) 1 of SEQ ID NO: 11, the heavy chain CDR 2 of SEQ ID NO:12, the heavy chain CDR 3 of SEQ ID NO: 13.
(ii) a peptide linker, in particular the peptide linker of SEQ ID NO:77.
(iii) a light chain variable domain (VL) comprising the light chain CDR 1 of SEQ ID NO:24, the light chain CDR 2 of SEQ ID NO:25 and the light chain CDR 3 of SEQ ID NO:26.
(iv) a peptide linker, in particular the peptide linker of SEQ ID NO:80.
(v) a transmembrane domain, in particular the transmembrane domain of SEQ ID NO:73.
(vi) a co-stimulatory signaling domain, in particular the co-stimulatory signaling domain of SEQ ID NO: 74 or 76, and
(vii) a stimulatory signaling domain, in particular the stimulatory signaling domain of SEQ ID NO:75.

In one embodiment, provided is a CAR capable of specific binding to a variant CH2-CH3 region comprising the amino acid substitution P329G according to EU numbering, wherein the CAR comprises in order from the N-terminus to the C-terminus:
(i) a heavy chain variable domain (VH) comprising the heavy chain complementarity determining region (CDR) 1 of SEQ ID NO:11, the heavy chain CDR 2 of SEQ ID NO: 19, the heavy chain CDR 3 of SEQ ID NO: 13.
(ii) a peptide linker, in particular the peptide linker of SEQ ID NO:77.
(iii) a light chain variable domain (VL) comprising the light chain CDR 1 of SEQ ID NO:24, the light chain CDR 2 of SEQ ID NO:25 and the light chain CDR 3 of SEQ ID NO:26.
(iv) a peptide linker, in particular the peptide linker of SEQ ID NO:80.
(v) a transmembrane domain, in particular the transmembrane domain of SEQ ID NO: 73.
(vi) a co-stimulatory signaling domain, in particular the co-stimulatory signaling domain of SEQ ID NO: 74 or 76, and
(vii) a stimulatory signaling domain, in particular the stimulatory signaling domain of SEQ ID NO:75.

In one embodiment, provided is a CAR capable of specific binding to a variant CH2-CH3 region comprising the amino acid substitution P329G according to EU numbering, wherein the CAR comprises in order from the N-terminus to the C-terminus:
(i) a heavy chain variable domain (VH).
(ii) a peptide linker, in particular the peptide linker of SEQ ID NO:77.
(iii) a light chain variable domain (VL) that is at least about 95%, 96%, 97%, 98%, 99% or 100% identical to the amino acid sequence of SEQ ID NO:23.
wherein the VH and VL domains are capable of forming an antigen binding moiety that binds to an Fc domain comprising the amino acid mutation P329G according to EU numbering,
(iv) a peptide linker, in particular the peptide linker of SEQ ID NO:80,
(v) a transmembrane domain, in particular a transmembrane domain that is at least about 95%, 96%, 97%, 98%, 99% or 100% identical to the amino acid sequence of SEQ ID NO:73,
(vi) a co-stimulatory signaling domain, in particular a co-stimulatory signaling domain that is at least about 95%, 96%, 97%, 98%, 99% or 100% identical to the amino acid sequence of SEQ ID NO: 74 or 76, and
(vii) a stimulatory signaling domain, in particular a stimulatory signaling domain that is at least about 95%, 96%, 97%, 98%, 99% or 100% identical to the amino acid sequence of SEQ ID NO:75.

In one embodiment, provided is a CAR capable of specific binding to a variant CH2-CH3 region comprising the amino acid substitution P329G according to EU numbering, wherein the CAR comprises in order from the N-terminus to the C-terminus:
(i) a heavy chain variable domain (VH) that is at least about 95%, 96%, 97%, 98%, 99% or 100% identical to the amino acid sequence of SEQ ID NO:10,
(ii) a peptide linker, in particular the peptide linker of SEQ ID NO:77,
(iii) a light chain variable domain (VL) that is at least about 95%, 96%, 97%, 98%, 99% or 100% identical to the amino acid sequence of SEQ ID NO:23,
(iv) a peptide linker, in particular the peptide linker of SEQ ID NO:80,
(v) a transmembrane domain, in particular a transmembrane domain that is at least about 95%, 96%, 97%, 98%, 99% or 100% identical to the amino acid sequence of SEQ ID NO:73,
(vi) a co-stimulatory signaling domain, in particular a co-stimulatory signaling domain that is at least about 95%, 96%, 97%, 98%, 99% or 100% identical to the amino acid sequence of SEQ ID NO: 74 or 76, and
(vii) a stimulatory signaling domain, in particular a stimulatory signaling domain that is at least about 95%, 96%, 97%, 98%, 99% or 100% identical to the amino acid sequence of SEQ ID NO:75.

In one embodiment, provided is a CAR capable of specific binding to a variant CH2-CH3 region comprising the amino acid substitution P329G according to EU numbering, wherein the CAR comprises in order from the N-terminus to the C-terminus:
(i) a heavy chain variable domain (VH) that is at least about 95%, 96%, 97%, 98%, 99% or 100% identical to the amino acid sequence of SEQ ID NO:18,
(ii) a peptide linker, in particular the peptide linker of SEQ ID NO:77,
(iii) a light chain variable domain (VL) that is at least about 95%, 96%, 97%, 98%, 99% or 100% identical to the amino acid sequence of SEQ ID NO:23,
(iv) a peptide linker, in particular the peptide linker of SEQ ID NO:80,
(v) a transmembrane domain, in particular a transmembrane domain that is at least about 95%, 96%, 97%, 98%, 99% or 100% identical to the amino acid sequence of SEQ ID NO:73,
(vi) a co-stimulatory signaling domain, in particular a co-stimulatory signaling domain that is at least about 95%, 96%, 97%, 98%, 99% or 100% identical to the amino acid sequence of SEQ ID NO: 74 or 76, and
(vii) a stimulatory signaling domain, in particular a stimulatory signaling domain that is at least about 95%, 96%, 97%, 98%, 99% or 100% identical to the amino acid sequence of SEQ ID NO:75.

In one embodiment, provided is a CAR capable of specific binding to a variant CH2-CH3 region comprising the amino acid substitution P329G according to EU numbering, wherein the CAR comprises in order from the N-terminus to the C-terminus:
(i) a heavy chain variable domain (VH) that is at least about 95%, 96%, 97%, 98%, 99% or 100% identical to the amino acid sequence of SEQ ID NO:20.
(ii) a peptide linker, in particular the peptide linker of SEQ ID NO:77.
(iii) a light chain variable domain (VL) that is at least about 95%, 96%, 97%, 98%, 99% or 100% identical to the amino acid sequence of SEQ ID NO:23.
(iv) a peptide linker, in particular the peptide linker of SEQ ID NO:80.
(v) a transmembrane domain, in particular a transmembrane domain that is at least about 95%, 96%, 97%, 98%, 99% or 100% identical to the amino acid sequence of SEQ ID NO:73.
(vi) a co-stimulatory signaling domain, in particular a co-stimulatory signaling domain that is at least about 95%, 96%, 97%, 98%, 99% or 100% identical to the amino acid sequence of SEQ ID NO: 76, and
(vii) a stimulatory signaling domain, in particular a stimulatory signaling domain that is at least about 95%, 96%, 97%, 98%, 99% or 100% identical to the amino acid sequence of SEQ ID NO:75.

In one embodiment, provided is a CAR capable of specific binding to a variant CH2-CH3 region comprising the amino acid substitution P329G according to EU numbering, wherein the CAR comprises in order from the N-terminus to the C-terminus:
(i) a heavy chain variable domain (VH) that is at least about 95%, 96%, 97%, 98%, 99% or 100% identical to the amino acid sequence of SEQ ID NO:20.
(ii) a peptide linker, in particular the peptide linker of SEQ ID NO:77.
(iii) a light chain variable domain (VL) that is at least about 95%, 96%, 97%, 98%, 99% or 100% identical to the amino acid sequence of SEQ ID NO:23.
(iv) a peptide linker, in particular the peptide linker of SEQ ID NO:80.
(v) a transmembrane domain, in particular a transmembrane domain that is at least about 95%, 96%, 97%, 98%, 99% or 100% identical to the amino acid sequence of SEQ ID NO:73.
(vi) a co-stimulatory signaling domain, in particular a co-stimulatory signaling domain that is at least about 95%, 96%, 97%, 98%, 99% or 100% identical to the amino acid sequence of SEQ ID NO: 74, and
(vii) a stimulatory signaling domain, in particular a stimulatory signaling domain that is at least about 95%, 96%, 97%, 98%, 99% or 100% identical to the amino acid sequence of SEQ ID NO:75.

In one embodiment, provided is a CAR capable of specific binding to a variant CH2-CH3 region comprising the amino acid substitution P329G according to EU numbering and capable of T cell activation, wherein the CAR comprises an amino acid sequence that is at least about 95%, 96%, 97%, 98%, 99% or 100% identical to the amino acid sequence of: SEQ ID NO:146. In one embodiment, provided is a CAR comprising the amino acid sequence of: SEQ ID NO: 146.

In one embodiment, provided is a CAR capable of specific binding to a variant CH2-CH3 region comprising the amino acid substitution P329G according to EU numbering and capable of T cell activation, wherein the CAR comprises an amino acid sequence that is at least about 95%, 96%, 97%, 98%, 99% or 100% identical to the amino acid sequence of: SEQ ID NO: 149. In one embodiment, provided is an CAR comprising the amino acid sequence of: SEQ ID NO:149.

In one embodiment, provided is a CAR capable of specific binding to a variant CH2-CH3 region comprising the amino acid substitution P329G according to EU numbering and capable of T cell activation, wherein the CAR comprises an amino acid sequence that is at least about 95%, 96%, 97%, 98%, 99% or 100% identical to the amino acid sequence of: SEQ ID NO: 151. In one embodiment, provided is an CAR comprising the amino acid sequence of: SEQ ID NO: 151.

In one embodiment, provided is a CAR capable of specific binding to a variant CH2-CH3 region comprising the amino acid substitution P329G according to EU numbering and capable of T cell activation, wherein the CAR comprises an amino acid sequence that is at least about 95%, 96%, 97%, 98%, 99% or 100% identical to the amino acid sequence of: SEQ ID NO: 154. In one embodiment, provided is an CAR comprising the amino acid sequence of: SEQ ID NO: 154.

In one embodiment, the CAR is fused to a reporter protein, particularly to GFP or enhanced analogs thereof. In one embodiment, the CAR is fused at the C-terminus to the N-terminus of eGFP (enhanced green fluorescent protein), optionally through a peptide linker as described herein. In a preferred embodiment, the peptide linker is GEGRGSLLTCGDVEENPGP (T2A) of SEQ ID NO:79.

### Recombinant CD3-TCR Complexes

In one particular embodiment, the membrane-anchored antigen binding (MAB) polypeptide comprises at least one recombinant CD3-TCR complex polypeptide.

CD3-TCR complexes (also sometimes referred to as TCR-CD3 complexes, see e.g. Dong et al., Nature (2019) 573 (7775): 546-552) are polypeptide complexes expressed at the cell surface of T cells that are involved in antigen-specific T cell activation. The structure and function of CD3-TCR complexes is reviewed e.g. in Mariuzza et al., J Biol Chem. 2020 Jan. 24; 295 (4): 914-925, which is hereby incorporated by reference in its entirety.

In mammals, CD3-TCR complexes comprise diverse TCR polypeptides that together form the heterodimeric TCR for antigen recognition (either TCRα and TCRβ, or TCRγ and TCRδ), provided in non-covalent association with invariant CD38, CD36, CD3γ and CD3ζ polypeptides. The classical, octameric CD3-TCR complex comprise a TCRα and TCRβ heterodimer (i.e. TCRαβ) or a TCRγ and TCRδ heterodimer (i.e. TCRγδ), a heterodimer comprising CD3ε and CD3δ (i.e. CD3δε), heterodimer comprising CD3ε and CD3γ (i.e. CD3γε)), and a CD3ζ homodimer (i.e. CD3ζζ). Such TCR-CD3 complexes may be represented respectively as CD3γε/CD3δε/CD3ζζ/TCRαβ and CD3γε/CD3δε/CD3ζζ/TCRγδ (see e.g. Zheng et al., Nature (2019) 573 (7775): 546-552).

In some embodiments, the CD3-TCR complex is a CD3-TCRα/β complex. In some embodiments, the CD3-TCR complex is a CD3-TCRγ/δ complex. A CD3-TCRα/β complex may comprise TCRα and TCRβ polypeptides, and also CD3γ, CD3ε, CD3δ and/or CD3ζ polypeptides. A CD3-TCRα/β complex may comprise or consist of CD3γε/CD3δε/CD3ζζ/TCRαβ. A CD3-TCRγ/δ complex may comprise TCRγ and TCRδ polypeptides, and also CD3γ, CD3ε, CD3δ and/or CD3ζ polypeptides. A CD3-TCRγ/δ complex may comprise or consist of CD3γε/CD3δε/CD3ζζ/TCRγ/δ.

Herein, a “CD3-TCR complex polypeptide” refers to a constituent polypeptide of a CD3-TCR complex. In some embodiments, a CD3-TCR complex polypeptide is selected from TCRα, TCRβ, TCRγ, TCRδ, TRAC, TRBC1, TRBC2, TRGC1, TRGC2, TRDC, CD3ε, CD3δ, CD3γ, CD3ζ and CD3η. In some embodiments, a CD3-TCR complex polypeptide is a recombinant CD3-TCR complex polypeptide as described herein.

In this specification, “TCRα”, “TCRβ”, “TCRγ”, “TCRδ”, “TRAC”, “TRBC1”, “TRBC2”, “TRGC1”, “TRGC2”, “TRDC”, “CD3ε”, “CD3δ”, “CD3γ”, “CD3” and “CD3” refer respectively to TCRα, TCRβ, TCRγ, TCRδ, TRAC, TRBC1, TRBC2, TRGC1, TRGC2, TRDC, CD3ε, CD3δ, CD3γ, CD3ζ and CD3η from any species, and include isoforms, fragments, variants or homologues from any species. In some embodiments, TCRα, TCRβ, TCRγ, TCRδ, TRAC, TRBC1, TRBC2, TRGC1, TRGC2, TRDC, CD3ε, CD3δ, CD3γ, CD3ζ or CD3η is from a mammal (e.g. a therian, placental, epitherian, preptotheria, archontan, primate (rhesus, cynomolgous, non-human primate or human)). In some embodiments, the TCRα, TCRβ, TCRγ, TCRδ, TRAC, TRBC1, TRBC2, TRGC1, TRGC2, TRDC, CD3ε, CD3δ, CD3γ, CD3ζ or CD3η is human.

As used herein, isoforms, fragments, variants or homologues of a given reference protein (e.g. TCRα, TCRβ, TCRγ, TCRδ, TRAC, TRBC1, TRBC2, TRGC1, TRGC2, TRDC, CD3ε, CD3δ, CD3γ, CD3ζ or CD3η) may be characterized as having at least 70% sequence identity, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to the amino acid sequence of the reference protein.

A “fragment” generally refers to a fraction of the reference protein. A “variant” generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An “isoform” generally refers to a variant of the reference protein expressed by the same species as the species of the reference protein. A “homologue” generally refers to a variant of the reference protein produced by a different species as compared to the species of the reference protein. Homologues include orthologues.

Isoforms, fragments, variants or homologues of a given reference protein may optionally be characterized as having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to the amino acid sequence of an immature or mature (i.e. after processing to remove signal peptide) form of a specified isoform of the relevant protein from a given species, e.g. human.

In some embodiments, TCRα comprises an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 157. In some embodiments. TRAC comprises, or consists of, an amino acid sequence having at least 70%, preferably one of ≥80%, ≥85%, ≥90%, ≥91%, ≥92%, ≥93%, ≥94%. ≥95%, ≥96%, ≥97%, ≥98%, ≥99% or 100% amino acid sequence identity to SEQ ID NO:157.

In some embodiments, TCRβ comprises an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:161 or 165. In some embodiments. TRBC1 comprises, or consists of, an amino acid sequence having at least 70%, preferably one of ≥80%, ≥85%, ≥90%, ≥91%, ≥92%, ≥93%. ≥94%, ≥95%, ≥96%, ≥97%, ≥98%, ≥99% or 100% amino acid sequence identity to SEQ ID NO:161. In some embodiments, TRBC2 comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:165.

In some embodiments, TCRγ comprises an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 169 or 173. In some embodiments. TRGC1 comprises, or consists of, an amino acid sequence having at least 70%, preferably one of ≥80%, ≥85%, ≥90%, ≥91%, ≥92%, ≥93%. ≥94%, ≥95%, ≥96%, ≥97%, ≥98%, ≥99% or 100% amino acid sequence identity to SEQ ID NO:169. In some embodiments, TRGC2 comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:173.

In some embodiments, TCRδ comprises an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 177. In some embodiments, TRDC comprises, or consists of, an amino acid sequence having at least 70%, preferably one of ≥80%, ≥85%, ≥90%, ≥91%, ≥92%, ≥93%, ≥94%. ≥95%, ≥96%, ≥97%, ≥98%, ≥99% or 100% amino acid sequence identity to SEQ ID NO:177.

In some embodiments, CD3ε comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 181 or 186.

In some embodiments, CD3δ comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 187 or 192.

In some embodiments, CD3γ comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 193 or 198.

In some embodiments, CD3ζ comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 199 or 204.

In some embodiments, CD3η comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:205 or 207.

In some embodiments, a recombinant CD3-TCR complex polypeptide according to the present disclosure comprises:
(i) an antigen-binding moiety, or a component thereof, as described herein; and
(ii) a CD3-TCR complex association domain as described herein.

In some embodiments, the amino acid sequence of the antigen-binding moiety/component thereof is N-terminal to the amino acid sequence of the CD3-TCR complex association domain, in the amino acid sequence of the recombinant CD3-TCR complex polypeptide. That is, in some embodiments, the recombinant CD3-TCR complex polypeptide comprises the structure: N term-[ . . . ]-[antigen-binding moiety/component thereof]-[CD3-TCR complex association domain]-[ . . . ]-C term.

In some embodiments, a recombinant CD3-TCR complex polypeptide according to the present disclosure does not comprise a domain or amino acid sequence comprising an immunoreceptor tyrosine-based activation motif (ITAM). ITAMs comprise an amino acid sequence according to YXXL/I (SEQ ID NO: 285), wherein “X” denotes any amino acid. In ITAM-containing proteins, sequences according to YXXL/I are often separated by 6 to 8 amino acids (i.e. they conform to the formula: YXXL/I (X)₆₋₈YXXL/I: SEQ ID NO:286). When phosphate groups are added to the tyrosine residue of an ITAM by tyrosine kinases, a signalling cascade is initiated within the cell. ITAM-containing sequences include the intracellular domains of CD3ζ, and FcγRI. In some embodiments, a recombinant CD3-TCR complex polypeptide according to the present disclosure does not comprise the amino acid sequence shown in SEQ ID NO: 203. In some embodiments, a recombinant CD3-TCR complex polypeptide does not comprise an amino acid sequence according to SEQ ID NO:286. In some embodiments, a recombinant CD3-TCR complex polypeptide does not comprise an amino acid sequence according to SEQ ID NO:285.

In some embodiments, a recombinant CD3-TCR complex polypeptide according to the present disclosure does not comprise a costimulatory sequence. As referred to herein, a “costimulatory sequence” refers to an amino acid sequence which provides for costimulation of an immune cell expressing the recombinant CD3-TCR complex polypeptide. Costimulation promotes proliferation and survival, and may also promote cytokine production, differentiation, cytotoxic function and memory formation. Molecular mechanisms of T cell costimulation are reviewed e.g. in Chen and Flics. (2013) Nat Rev Immunol 13 (4): 227-242. A costimulatory sequence may be, or may be derived from the intracellular domain of a costimulatory protein. Costimulatory proteins include CD28, 4-1BB, ICOS, CD27, OX40, HVEM, CD2, SLAM, TIM-1, CD30, GITR, DR3, CD226 and LIGHT. In some embodiments, a recombinant CD3-TCR complex polypeptide according to the present disclosure does not comprise the amino acid sequence shown in SEQ ID NO: 138 (the intracellular domain of human 4-1BB).

The recombinant CD3-TCR complex polypeptides of the present disclosure comprise a CD3-TCR complex association domain. The essential function of the CD3-TCR complex association domain is to provide for the formation of polypeptide complexes comprising the recombinant CD3-TCR complex polypeptide according to the present disclosure, and one or more CD3-TCR complex polypeptides.

A “CD3-TCR complex association domain” refers to a domain through which a polypeptide comprising the domain is able to associate with a CD3-TCR complex polypeptide (e.g. a CD3-TCR complex polypeptide as described hereinabove). Thus, a CD3-TCR complex association domain according to the present disclosure comprises or consists of an amino acid sequence conferring to a polypeptide comprising the domain the ability to associate with a CD3-TCR complex polypeptide, to form a polypeptide complex comprising the CD3-TCR complex polypeptide and the polypeptide bearing the CD3-TCR complex association domain.

Association between the CD3-TCR complex association domain/polypeptide comprising the CD3-TCR complex association domain and the CD3-TCR complex polypeptide may be characterized by non-covalent, protein:protein interaction. In some embodiments, the association comprises electrostatic interaction (e.g. ionic bonding, hydrogen bonding) and/or Van der Waals forces.

In some embodiments, the CD3-TCR complex association domain is, or is derived from the amino acid sequence of a CD3-TCR complex polypeptide. It will be appreciated that the CD3-TCR complex association domain may be, or may be derived from the region of a CD3-TCR complex polypeptide through which the CD3-TCR complex polypeptide interacts with other CD3-TCR complex polypeptides to form polypeptide complexes. In some embodiments, the CD3-TCR complex association domain is, or is derived from, the amino acid sequence of the region of a CD3-TCR complex polypeptide required for association between the CD3-TCR complex polypeptide other CD3-TCR complex polypeptides, to form a polypeptide complex comprising such polypeptides.

The region of a CD3-TCR complex polypeptide required for such interaction can be determined by site-directed mutagenesis and/or truncation studies, in which the amino acid sequence of the CD3-TCR complex polypeptide is altered or truncated, and the effect of such alteration/truncation on its ability to associate with other CD3-TCR complex polypeptides is evaluated. Suitable techniques for investigating such protein:protein interaction include e.g. resonance energy transfer techniques such as fluorescence resonance energy transfer (FRET) and Bioluminescence Resonance Energy Transfer (BRET), using appropriate labeled interaction partners, e.g. as described in Ciruela. Curr. Opin. Biotechnol. (2008) 19 (4): 338-43.

As used herein, polypeptides, domains and amino acid sequences which are “derived from” a reference polypeptide/domain/amino acid sequence have at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to the amino acid sequence of the reference polypeptide/domain/amino acid sequence. Polypeptides, domains and amino acid sequences which are “derived from” a reference polypeptide/domain/amino acid sequence preferably retain the functional and/or structural properties of the reference polypeptide/domain/amino acid sequence.

In some embodiments, the CD3-TCR complex association domain comprises modification to promote association with a CD3-TCR complex polypeptide. In some embodiments, the CD3-TCR complex association domain comprises modification to promote heteromerisation, i.e. association with a non-identical CD3-TCR complex polypeptide.

As used herein, a “modification” refers to a difference relative to a reference amino acid sequence. A reference amino acid sequence may be the amino acid sequence encoded by the most common nucleotide sequence of the gene encoding the relevant protein. In embodiments herein (and also in the art more generally), a “modification” may also be referred to as a “substitution” or a “mutation”. A modification typically comprises substitution of an amino acid residue. Substitution of an amino acid residue comprises substitution of an amino acid residue a non-identical “replacement” amino acid residue. A replacement amino acid residue of a modification according to the present disclosure may be a naturally-occurring amino acid residue (i.e. encoded by the genetic code) which is non-identical to the amino acid residue at the relevant position of the amino acid sequence prior to modification, selected from: alanine (Ala), arginine (Arg), asparagine (Asn), aspartic acid (Asp), cysteine (Cys), glutamine (Gln), glutamic acid (Glu), glycine (Gly), histidine (His), isoleucine (Ile): leucine (Leu), lysine (Lys), methionine (Met), phenylalanine (Phe), proline (Pro), serine (Ser), threonine (Thr), tryptophan (Trp), tyrosine (Tyr), and valine (Val). In some embodiments, a replacement amino acid residue of a modification may be a non-naturally occurring amino acid residue—i.e. an amino acid residue other than those recited in the preceding sentence. Examples of non-naturally occurring amino acid residues include norleucine, ornithine, norvaline, homoserine, aib, and other amino acid residue analogues such as those described in Ellman, et al., Meth. Enzym. 202 (1991) 301-336.

By way of illustration, in embodiments herein, a CD3-TCR complex association domain derived from TRAC comprises a modification to replace the threonine residue at position 47 (numbered relative to SEQ ID NO: 157) with a cysteine residue (SEQ ID NO:208), and a CD3-TCR complex association domain derived from TCRβ comprises a modification to replace the serine residue at position 56 (numbered relative to SEQ ID NO: 161) with a cysteine residue (SEQ ID NO:209). The introduction of these cysteine residues promotes heteromerisation between the modified domains via formation of an interchain disulfide bridge.

Embodiments in which the CD3-TCR complex association domain further comprises modification to promote association with a CD3-TCR complex polypeptide are contemplated in particular in connection with aspects and embodiments of the present disclosure wherein the recombinant CD3-TCR complex polypeptide comprising such a CD3-TCR complex association domain is provided to be employed with another recombinant CD3-TCR complex polypeptide. For example, such CD3-TCR complex association domains are contemplated, in particular, to be employed in a first recombinant CD3-TCR complex polypeptide and/or a second recombinant CD3-TCR complex polypeptide of a polypeptide complex of the present disclosure comprising such recombinant CD3-TCR complex polypeptides.

In some embodiments, the CD3-TCR complex association domain is, or is derived from, the CD3-TCR complex association domain of CD38. In some embodiments, the CD3-TCR complex association domain is, or is derived from the region of CD3ε required for association with CD3γ and/or CD3δ (i.e. to form a CD3ε:CD3γ polypeptide complex, or a CD3ε:CD3δ polypeptide complex). In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%. ≥97%, ≥98%, ≥99% or 100% amino acid sequence identity to SEQ ID NO:186.

In some embodiments, the CD3-TCR complex association domain is, or is derived from, the CD3-TCR complex association domain of TRAC. In some embodiments, the CD3-TCR complex association domain is, or is derived from the region of TRAC required for association with TCRβ, TRBC1 and/or TRBC2 (i.e. to form a TRAC:TCRβ polypeptide complex, or a TRAC:TRBC1 polypeptide complex, or a TRAC:TRBC2 polypeptide complex). In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 157. In some embodiments, the CD3-TCR complex association domain is derived from TRAC, and further comprises modification to promote association with another CD3-TCR complex polypeptide. In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98% or ≥99% amino acid sequence identity to SEQ ID NO: 157, and comprises a cysteine residue at the position corresponding to position 47 numbered according to SEQ ID NO: 157. In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:208.

In some embodiments, the CD3-TCR complex association domain is, or is derived from, the CD3-TCR complex association domain of TRBC1. In some embodiments, the CD3-TCR complex association domain is, or is derived from the region of TRBC1 required for association with TCRα and/or TRAC (i.e. to form a TRBC1: TCRα polypeptide complex, or a TRBC1: TRAC polypeptide complex). In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:161. In some embodiments, the CD3-TCR complex association domain is derived from TRBC1, and further comprises modification to promote association with another CD3-TCR complex polypeptide. In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%. ≥94%, ≥95%, ≥96%, ≥97%, ≥98% or ≥99% amino acid sequence identity to SEQ ID NO: 161, and comprises a cysteine residue at the position corresponding to position 56 numbered according to SEQ ID NO: 161. In some embodiments, the CD3-TCR complex association domain comprises, or consists of, an amino acid sequence having at least 60%, preferably one of ≥70%, ≥75%, ≥80%, ≥85%, ≥90%, ≥91%. ≥92%, ≥93%, ≥94%, ≥95%, ≥96%, ≥97%, ≥98%, ≥99% or 100% amino acid sequence identity to SEQ ID NO: 209.

In some embodiments, the CD3-TCR complex association domain is, or is derived from, the CD3-TCR complex association domain of TRBC2. In some embodiments, the CD3-TCR complex association domain is, or is derived from the region of TRBC2 required for association with TCRα and/or TRAC (i.e. to form a TRBC2: TCRα polypeptide complex, or a TRBC2: TRAC polypeptide complex). In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%. ≥94%, ≥95%, ≥96%, ≥97%, ≥98%, ≥99% or 100% amino acid sequence identity to SEQ ID NO: 165. In some embodiments, the CD3-TCR complex association domain is derived from TRBC2, and further comprises modification to promote association with another CD3-TCR complex polypeptide. In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%. ≥94%, ≥95%, ≥96%, ≥97%, ≥98% or ≥99% amino acid sequence identity to SEQ ID NO: 165, and comprises a cysteine residue at the position corresponding to position 56 numbered according to SEQ ID NO: 165. In some embodiments, the CD3-TCR complex association domain comprises, or consists of, an amino acid sequence having at least 60%, preferably one of ≥70%, ≥75%, ≥80%, ≥85%, ≥90%, ≥91%. ≥92%, ≥93%, ≥94%, ≥95%, ≥96%, ≥97%, ≥98%, ≥99% or 100% amino acid sequence identity to SEQ ID NO: 210.

In aspects and embodiments of the present disclosure, a recombinant CD3-TCR complex polypeptide comprises a component of an antigen-binding moiety as described hereinabove. This is particularly the case when it is contemplated to employ the recombinant CD3-TCR complex polypeptide with another, non-identical and complementary recombinant CD3-TCR complex polypeptide. In such aspects and embodiments, the two non-identical and complementary polypeptides preferably associate with one another to form a polypeptide complex comprising the antigen-binding moiety. That is, association between the recombinant CD3-TCR complex polypeptides reconstitutes the functional antigen-binding moiety.

By way of illustration, in embodiments described herein, a recombinant CD3-TCR complex polypeptide comprises the VH region of an antigen-binding moiety specific for a variant Fc domain as described herein above and the ECD, TMD and ICD of TRAC (T47C), and it is contemplated to employ this recombinant CD3-TCR complex polypeptide in conjunction with a recombinant CD3-TCR complex polypeptide comprising the VL region of the antigen-binding moiety specific for a variant Fc domain, and the ECD, TMD and ICD of TRBC1 (S56C). When expressed in a cell, the two recombinant CD3-TCR complex polypeptides associate to form a polypeptide complex comprising an Fv specific for a variant Fc domain, formed by the VH region from the first polypeptide, and the VL region from the second polypeptide.

In some aspects and embodiments of the present disclosure, first and second components of an antigen-binding moiety as described hereinabove are provided. In accordance with such aspects and embodiments, it will be appreciated that the first and second components of an antigen-binding moiety are complementary, and capable of associating to form the (complete, functional) antigen-binding moiety.

In some embodiments according to the present disclosure, a component of an antigen-binding moiety may be or comprise the VH region of an antigen-binding moiety specific for a variant Fc domain (e.g. as described herein) as described hereinabove. In some embodiments, a component of an antigen-binding moiety may be or comprise the VL region of an antigen-binding moiety specific for a variant Fc domain (e.g. as described herein). In preferred embodiments, the VH region and VL region may be from the same antigen-binding moiety.

In some embodiments, a component of an antigen-binding moiety comprises, or consists of, a VH as described herein above. In some embodiments, a component of an antigen-binding moiety comprises, or consists of, a VL as described herein above. In some embodiments, a component of an antigen-binding moiety comprises one or more antibody heavy chain constant regions (CH). In some embodiments, a component of an antigen-binding moiety comprises one or more antibody light chain constant regions (CL). In some embodiments, a component of an antigen-binding moiety comprises a CH1, CH2 region and/or a CH3 region of an immunoglobulin (Ig).

In some embodiments, a CD3-TCR complex polypeptide according to the present disclosure comprises or consists of one of the following structures:
N term-[signal peptide]-[antigen binding moiety, or a component thereof]-[CD3-TCR complex association domain]-C term
N term-[antigen binding moiety, or a component thereof]-[CD3-TCR complex association domain]-C term
N term-[signal peptide]-[antigen binding moiety, or a component thereof]-[CD3-TCR complex association domain]-[cleavage site]-[detectable moiety]-C term
N term-[antigen binding moiety, or a component thereof]-[CD3-TCR complex association domain]-[cleavage site]-[detectable moiety]-C term

In some embodiments, a composite polypeptide according to the present disclosure comprises or consists of one of the following structures:
N term-[signal peptide]-[antigen-binding moiety component]-[CD3-TCR complex association domain]-[cleavage site]-[signal peptide]-[antigen-binding moiety component]-[CD3-TCR complex association domain]-C term
N term-[signal peptide]-[antigen-binding moiety component]-[CD3-TCR complex association domain]-[cleavage site]-[signal peptide]-[antigen-binding moiety component]-[CD3-TCR complex association domain]-[cleavage site]-[detectable moiety]-C term

In some embodiments, a CD3-TCR complex polypeptide according to the present disclosure comprises or consists of (e.g. from N-terminus to C-terminus):
(1) (i) an amino acid sequence encoding a signal peptide, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:135;
   (ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 1; and
   (iii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column B of Table 1;
   wherein the sequence selected from Column A of Table 1 and the sequence selected from Column B of Table 1 are selected from the same row of Table 1.
(2) (i) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 1; and
   (ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column B of Table 1;
   wherein the sequence selected from Column A of Table 1 and the sequence selected from Column B of Table 1 are selected from the same row of Table 1.
(3) (i) an amino acid sequence encoding a signal peptide, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:135;
   (ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 1;
   (iii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column B of Table 1;
   (iv) an amino acid sequence encoding a cleavage site, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:141; and
   (v) an amino acid sequence encoding a detectable moiety, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:140;
   wherein the sequence selected from Column A of Table 1 and the sequence selected from Column B of Table 1 are selected from the same row of Table 1.
(4) (i) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 1;
   (ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column B of Table 1;
   (iii) an amino acid sequence encoding a cleavage site, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:141; and
   (iv) an amino acid sequence encoding a detectable moiety, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:140;
   wherein the sequence selected from Column A of Table 1 and the sequence selected from Column B of Table 1 are selected from the same row of Table 1.

TABLE 1

| Row | Column A | Column B |
|---|---|---|
| 1 | SEQ ID NO: 33 | SEQ ID NO: 186 |
| 2 | SEQ ID NO: 34 | SEQ ID NO: 186 |
| 3 | SEQ ID NO: 35 | SEQ ID NO: 186 |
| 4 | SEQ ID NO: 10 | SEQ ID NO: 157 |
| 5 | SEQ ID NO: 10 | SEQ ID NO: 208 |
| 6 | SEQ ID NO: 10 | SEQ ID NO: 161 |
| 7 | SEQ ID NO: 10 | SEQ ID NO: 209 |
| 8 | SEQ ID NO: 10 | SEQ ID NO: 165 |
| 9 | SEQ ID NO: 10 | SEQ ID NO: 210 |
| 10 | SEQ ID NO: 18 | SEQ ID NO: 157 |
| 11 | SEQ ID NO: 18 | SEQ ID NO: 208 |
| 12 | SEQ ID NO: 18 | SEQ ID NO: 161 |
| 13 | SEQ ID NO: 18 | SEQ ID NO: 209 |
| 14 | SEQ ID NO: 18 | SEQ ID NO: 165 |
| 15 | SEQ ID NO: 18 | SEQ ID NO: 210 |
| 16 | SEQ ID NO: 20 | SEQ ID NO: 157 |
| 17 | SEQ ID NO: 20 | SEQ ID NO: 208 |
| 18 | SEQ ID NO: 20 | SEQ ID NO: 161 |
| 19 | SEQ ID NO: 20 | SEQ ID NO: 209 |
| 20 | SEQ ID NO: 20 | SEQ ID NO: 165 |
| 21 | SEQ ID NO: 20 | SEQ ID NO: 210 |
| 22 | SEQ ID NO: 23 | SEQ ID NO: 157 |
| 23 | SEQ ID NO: 23 | SEQ ID NO: 208 |
| 24 | SEQ ID NO: 23 | SEQ ID NO: 161 |
| 25 | SEQ ID NO: 23 | SEQ ID NO: 209 |
| 26 | SEQ ID NO: 23 | SEQ ID NO: 165 |
| 27 | SEQ ID NO: 23 | SEQ ID NO: 210 |

In some embodiments, a CD3-TCR complex polypeptide according to the present disclosure comprises or consists of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to one of SEQ ID NOs: 211 to 255. In some embodiments, a CD3-TCR complex polypeptide according to the present disclosure comprises or consists of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:222.

In some embodiments, a composite polypeptide according to the present disclosure comprises or consists of (e.g. from N-terminus to C-terminus):
(1) (i) an amino acid sequence encoding a signal peptide, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:135;
   (ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 2; and
   (iii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column B of Table 2;
   (iv) an amino acid sequence encoding a cleavage site, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:141;
   (v) an amino acid sequence encoding a signal peptide, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:135;
   (vi) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column C of Table 2; and
   (vii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column D of Table 2;
   wherein the sequence selected from Column A of Table 2 and the sequence selected from Column B of Table 2 and the sequence selected from Column C of Table 2 and the sequence selected from Column D of Table 2 are selected from the same row of Table 2.
(2) (i) an amino acid sequence encoding a signal peptide, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:135;
   (ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 2; and
   (iii) an amino acid sequence having at least 70%, preferably one of ≥80%, ≥85%, ≥90%, ≥91%, ≥92%, 293%, ≥94%, ≥95%, ≥96%, ≥97%, ≥98%, ≥99% or 100% amino acid sequence identity to a sequence selected from of Column B of Table 2;
   (iv) an amino acid sequence encoding a cleavage site, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:141;
   (v) an amino acid sequence encoding a signal peptide, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:135;
   (vi) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column C of Table 2;
   (vii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column D of Table 2;
   (viii) an amino acid sequence encoding a cleavage site, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:141; and
   (ix) an amino acid sequence encoding a detectable moiety, e.g. an amino acid sequence having at least 70%, preferably one of ≥80%, ≥85%, ≥90%, ≥91%, ≥92%, ≥93%, 294%, ≥95%, ≥96%, ≥97%, ≥98%, ≥99% or 100% amino acid sequence identity to SEQ ID NO:140;
   wherein the sequence selected from Column A of Table 2 and the sequence selected from Column B of Table 2 and the sequence selected from Column C of Table 2 and the sequence selected from Column D of Table 2 are selected from the same row of Table 2.

TABLE 2

| Row | Column A | Column B | Column C | Column D |
|---|---|---|---|---|
| 1 | SEQ ID NO: 10 | SEQ ID NO: 157 | SEQ ID NO: 23 | SEQ ID NO: 161 |
| 2 | SEQ ID NO: 18 | SEQ ID NO: 157 | SEQ ID NO: 23 | SEQ ID NO: 161 |
| 3 | SEQ ID NO: 20 | SEQ ID NO: 157 | SEQ ID NO: 23 | SEQ ID NO: 161 |
| 4 | SEQ ID NO: 23 | SEQ ID NO: 157 | SEQ ID NO: 10 | SEQ ID NO: 161 |
| 5 | SEQ ID NO: 23 | SEQ ID NO: 157 | SEQ ID NO: 18 | SEQ ID NO: 161 |
| 6 | SEQ ID NO: 23 | SEQ ID NO: 157 | SEQ ID NO: 20 | SEQ ID NO: 161 |
| 9 | SEQ ID NO: 10 | SEQ ID NO: 157 | SEQ ID NO: 23 | SEQ ID NO: 161 |
| 10 | SEQ ID NO: 18 | SEQ ID NO: 157 | SEQ ID NO: 23 | SEQ ID NO: 161 |
| 11 | SEQ ID NO: 20 | SEQ ID NO: 157 | SEQ ID NO: 23 | SEQ ID NO: 161 |
| 12 | SEQ ID NO: 23 | SEQ ID NO: 157 | SEQ ID NO: 10 | SEQ ID NO: 161 |
| 13 | SEQ ID NO: 23 | SEQ ID NO: 157 | SEQ ID NO: 18 | SEQ ID NO: 161 |
| 14 | SEQ ID NO: 23 | SEQ ID NO: 157 | SEQ ID NO: 20 | SEQ ID NO: 161 |
| 17 | SEQ ID NO: 10 | SEQ ID NO: 208 | SEQ ID NO: 23 | SEQ ID NO: 209 |
| 18 | SEQ ID NO: 18 | SEQ ID NO: 208 | SEQ ID NO: 23 | SEQ ID NO: 209 |
| 19 | SEQ ID NO: 20 | SEQ ID NO: 208 | SEQ ID NO: 23 | SEQ ID NO: 209 |
| 20 | SEQ ID NO: 23 | SEQ ID NO: 208 | SEQ ID NO: 10 | SEQ ID NO: 209 |
| 21 | SEQ ID NO: 23 | SEQ ID NO: 208 | SEQ ID NO: 18 | SEQ ID NO: 209 |
| 22 | SEQ ID NO: 23 | SEQ ID NO: 208 | SEQ ID NO: 20 | SEQ ID NO: 209 |
| 25 | SEQ ID NO: 10 | SEQ ID NO: 208 | SEQ ID NO: 23 | SEQ ID NO: 210 |
| 26 | SEQ ID NO: 18 | SEQ ID NO: 208 | SEQ ID NO: 23 | SEQ ID NO: 210 |
| 27 | SEQ ID NO: 20 | SEQ ID NO: 208 | SEQ ID NO: 23 | SEQ ID NO: 210 |
| 28 | SEQ ID NO: 23 | SEQ ID NO: 208 | SEQ ID NO: 10 | SEQ ID NO: 210 |
| 29 | SEQ ID NO: 23 | SEQ ID NO: 208 | SEQ ID NO: 18 | SEQ ID NO: 210 |
| 30 | SEQ ID NO: 23 | SEQ ID NO: 208 | SEQ ID NO: 20 | SEQ ID NO: 210 |
| 33 | SEQ ID NO: 10 | SEQ ID NO: 161 | SEQ ID NO: 23 | SEQ ID NO: 157 |
| 34 | SEQ ID NO: 18 | SEQ ID NO: 161 | SEQ ID NO: 23 | SEQ ID NO: 157 |
| 35 | SEQ ID NO: 20 | SEQ ID NO: 161 | SEQ ID NO: 23 | SEQ ID NO: 157 |
| 36 | SEQ ID NO: 23 | SEQ ID NO: 161 | SEQ ID NO: 10 | SEQ ID NO: 157 |
| 37 | SEQ ID NO: 23 | SEQ ID NO: 161 | SEQ ID NO: 18 | SEQ ID NO: 157 |
| 38 | SEQ ID NO: 23 | SEQ ID NO: 161 | SEQ ID NO: 20 | SEQ ID NO: 157 |
| 41 | SEQ ID NO: 10 | SEQ ID NO: 165 | SEQ ID NO: 23 | SEQ ID NO: 157 |
| 42 | SEQ ID NO: 18 | SEQ ID NO: 165 | SEQ ID NO: 23 | SEQ ID NO: 157 |
| 43 | SEQ ID NO: 20 | SEQ ID NO: 165 | SEQ ID NO: 23 | SEQ ID NO: 157 |
| 44 | SEQ ID NO: 23 | SEQ ID NO: 165 | SEQ ID NO: 10 | SEQ ID NO: 157 |
| 45 | SEQ ID NO: 23 | SEQ ID NO: 165 | SEQ ID NO: 18 | SEQ ID NO: 157 |
| 46 | SEQ ID NO: 23 | SEQ ID NO: 165 | SEQ ID NO: 20 | SEQ ID NO: 157 |
| 49 | SEQ ID NO: 10 | SEQ ID NO: 209 | SEQ ID NO: 23 | SEQ ID NO: 208 |
| 50 | SEQ ID NO: 18 | SEQ ID NO: 209 | SEQ ID NO: 23 | SEQ ID NO: 208 |
| 51 | SEQ ID NO: 20 | SEQ ID NO: 209 | SEQ ID NO: 23 | SEQ ID NO: 208 |
| 52 | SEQ ID NO: 23 | SEQ ID NO: 209 | SEQ ID NO: 10 | SEQ ID NO: 208 |
| 53 | SEQ ID NO: 23 | SEQ ID NO: 209 | SEQ ID NO: 18 | SEQ ID NO: 208 |
| 54 | SEQ ID NO: 23 | SEQ ID NO: 209 | SEQ ID NO: 20 | SEQ ID NO: 208 |
| 57 | SEQ ID NO: 10 | SEQ ID NO: 210 | SEQ ID NO: 23 | SEQ ID NO: 208 |
| 58 | SEQ ID NO: 18 | SEQ ID NO: 210 | SEQ ID NO: 23 | SEQ ID NO: 208 |
| 59 | SEQ ID NO: 20 | SEQ ID NO: 210 | SEQ ID NO: 23 | SEQ ID NO: 208 |
| 60 | SEQ ID NO: 23 | SEQ ID NO: 210 | SEQ ID NO: 10 | SEQ ID NO: 208 |
| 61 | SEQ ID NO: 23 | SEQ ID NO: 210 | SEQ ID NO: 18 | SEQ ID NO: 208 |
| 62 | SEQ ID NO: 23 | SEQ ID NO: 210 | SEQ ID NO: 20 | SEQ ID NO: 208 |

In some embodiments, a composite polypeptide according to the present disclosure comprises or consists of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to one of SEQ ID NOs: 256 to 279. In some embodiments, a composite polypeptide according to the present disclosure comprises or consists of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:260. In some embodiments, a composite polypeptide according to the present disclosure comprises or consists of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:266.

In some embodiments, a polypeptide complex according to the present disclosure comprises a CD3-TCR complex polypeptide according to an embodiment described herein.

In some embodiments, a polypeptide complex according to the present disclosure comprises:
(a) a polypeptide comprising (e.g. from N-terminus to C-terminus):
   (i) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 2; and
   (ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column B of Table 2;
      and
(b) a polypeptide comprising (e.g. from N-terminus to C-terminus):
   (i) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column C of Table 2; and
   (ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column D of Table 2;
      wherein the sequence selected from Column A of Table 2 and the sequence selected from Column B of Table 2 and the sequence selected from Column C of Table 2 and the sequence selected from Column D of Table 2 are selected from the same row of Table 2.

In some embodiments, a polypeptide complex according to the present disclosure comprises:
(1) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:227; and
   (ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:255;
or
(2) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:231; and
   (ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:255;
or
(3) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:235; and
   (ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:255;
or
(4) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:239; and
   (ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:243;
or
(5) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:239; and
   (ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:247;
or
(6) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:239; and
   (ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:251.

In preferred embodiments, a polypeptide complex according to the present disclosure comprises:
(i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:235; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:255.

In preferred embodiments, a polypeptide complex according to the present disclosure comprises:
(i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:239; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:251.

### Variant Fc Domain-Bearing Antigen-Binding Molecule (Targeting Antibody)

In the therapeutic/prophylactic intervention of the present disclosure, the variant Fc domain-bearing molecule (e.g. the recombinant Fc-IL2v polypeptide) serves as an adaptor molecule, to direct the activity of a cytokine (e.g. IL2 and/or variants thereof) to a cell expressing the MAB polypeptide (complex) as herein described.

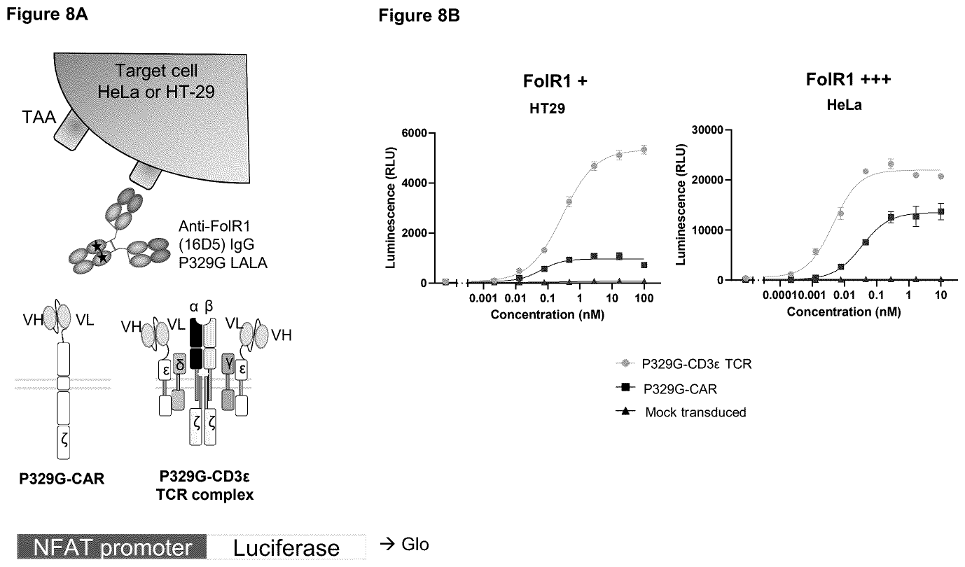

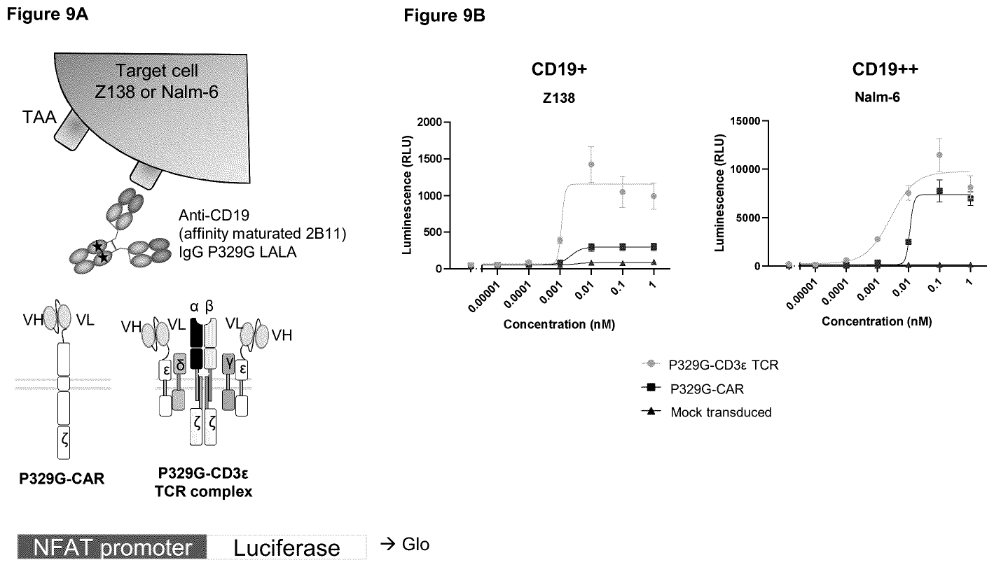

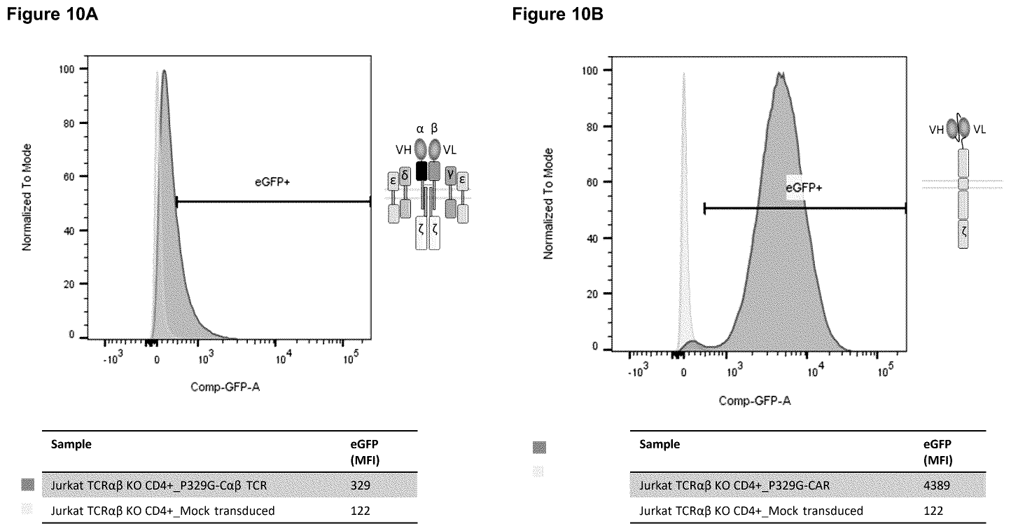

In some aspect, in the therapeutic/prophylactic intervention of the present disclosure, another variant Fc domain-bearing molecule (e.g. a targeting antibody) can simultaneously serve as an adaptor molecule to direct the activity of the cell expressing the MAB polypeptide (complex) according to the present disclosure against an antigen on a target cell (e.g. on a tumor cell). That is, in embodiments wherein the cell is an immune cell (e.g. a T cell), a variant Fc domain-bearing antigen-binding molecule can direct a cell-mediated immune response (e.g. a T cell-mediated immune response) against cells expressing the antigen to which the antigen-binding molecule binds (see for example FIGS. 8 and 9). Such variant Fc domain-bearing antigen-binding molecule is herein referred to as “targeting antibody”.

By way of illustration, in the Examples of the present disclosure, a T cell expressing a CD3-TCR polypeptide complex comprising a recombinant CD3-TCR complex polypeptide according to SEQ ID NO: 222 is employed with an anti-FolR1 antibody comprising an Fc domain comprising P329G, such that the T cells are directed against FolR1-expressing cells. By way of further illustration, in the Examples of the present disclosure, a T cell expressing a CD3-TCR polypeptide complex comprising a recombinant CD3-TCR complex polypeptide according to SEQ ID NO:222 is employed with an anti-CD19 antibody comprising an Fc domain comprising P329G, such that the T cells are directed against CD19-expressing cells. By way of further illustration, in the Examples of the present disclosure, a T cell expressing a CD3-TCR polypeptide complex comprising (i) a recombinant CD3-TCR complex polypeptide according to SEQ ID NO:235 and (ii) a recombinant CD3-TCR complex polypeptide according to SEQ ID NO:255 is employed with an anti-FolR1 antibody comprising an Fc domain comprising P329G, such that the T cells are directed against FolR1-expressing cells. By way of further illustration, in the Examples of the present disclosure, a T cell expressing a CD3-TCR polypeptide complex comprising (i) a recombinant CD3-TCR complex polypeptide according to SEQ ID NO:235 and (ii) a recombinant CD3-TCR complex polypeptide according to SEQ ID NO:255 is employed with an anti-CD19 antibody comprising an Fc domain comprising P329G, such that the T cells are directed against CD19-expressing cells.

A targeting antibody as herein described may comprise any of the Fc domain polypeptide as described above (e.g. in section Fc domain polypeptides). Additionally, the targeting antibody is capable of binding to a target antigen.

In some aspects, the targeting antibody comprises at least one antigen-binding moiety. As used herein, an “antigen-binding moiety” refers to a moiety that binds to a given target antigen. Antigen-binding moieties include antibodies (i.e. immunoglobulins (Igs)), and antigen-binding fragments and derivatives thereof. In some embodiments, an antigen-binding moiety according to the present disclosure comprises, or consists of, a monoclonal antibody, a monospecific antibody, a multispecific (e.g., bispecific, trispecific, etc.) antibody, a variable fragment (Fv) moiety, a single-chain Fv (scFv) moiety, a fragment antigen-binding (Fab) moiety, a single-chain Fab moiety (scFab), a crossFab moiety, a Fab moiety, a Fab-SH moiety, a F(ab′)₂ moiety, a diabody moiety, a triabody moiety, an scFv-Fc moiety, a minibody moiety, a heavy chain only antibody (HCAb) moiety, or a single domain antibody (dAb. VHH) moiety. Further included are antigen-binding peptides/polypeptides such as peptide aptamers, thioredoxins, anticalins, Kunitz domains, avimers, knottins, fynomers, atrimers. DARPins, affibodys, affilins, armadillo repeat proteins (ArmRPs), OBodys and adnectins (reviewed e.g. in Reverdatto et al., Curr Top Med Chem. 2015; 15 (12): 1082-1101, which is hereby incorporated by reference in its entirety (see also e.g. Boersma et al., J Biol Chem (2011) 286:41273-85 and Emanuel et al., Mabs (2011) 3:38-48)). Further included are target antigen-binding nucleic acids, e.g. nucleic acid aptamers (reviewed, for example, in Zhou and Rossi Nat Rev Drug Discov. 2017 16 (3): 181-202). Further included are target antigen-binding small molecules (e.g. low molecular weight (<1000 daltons, typically between ˜300-700 daltons) organic compounds).

The antigen-binding moieties of the targeting antibodies of the present disclosure is capable of binding to a target antigen. The antigen-binding moieties preferably display specific binding to the target antigen. As used herein. “specific binding” refers to binding which is selective for the target antigen, and which can be discriminated from non-specific binding to non-target antigen. An antigen-binding moiety that specifically binds to a given target antigen preferably binds the target antigen with greater affinity, and/or with greater duration than it binds to other, non-target antigens. The ability of a given moiety to bind specifically to a given target antigen can be determined by analysis according to methods known in the art, such as by ELISA. Surface Plasmon Resonance (SPR; see e.g. Hearty et al., Methods Mol Biol (2012) 907:411-442). Bio-Layer Interferometry (BLI; see e.g. Lad et al., (2015) J Biomol Screen 20 (4): 498-507), flow cytometry, or by a radiolabeled antigen-binding assay (RIA) enzyme-linked immunosorbent assay. Through such analysis binding to a given target antigen can be measured and quantified. In some embodiments, the level of binding may be the response detected in a given assay.

In some embodiments, the antigen-binding moiety binds to a target antigen with an affinity (e.g. determined by SPR or BLI) in the micromolar range, i.e. K_{D}=9.9×10⁻⁴ to 1×10⁻⁶ M. In some embodiments, the antigen-binding moiety binds to a target antigen with sub-micromolar affinity, i.e. K_{D}<1×10⁻⁶ M. In some embodiments, the antigen-binding moiety binds to the target antigen with an affinity in the nanomolar range, i.e. K_{D}=9.9×10⁻⁷ to 1×10⁻⁹ M. In some embodiments, the antigen-binding moiety binds to a target antigen with sub-nanomolar affinity, i.e. K_{D}<1×10⁻⁹ M. In some embodiments, the antigen-binding moiety binds to target antigen with an affinity in the picomolar range, i.e. K_{D}=9.9×10⁻¹⁰ to 1×10⁻¹² M. In some embodiments, the antigen-binding moiety binds to a target antigen with sub-picomolar affinity, i.e. K_{D}<1×10⁻¹² M.

The target antigen may be any target antigen expressed by a cell that it is desired to kill/deplete in order to attain a therapeutic/prophylactic effect. In some embodiments, the target antigen is an antigen whose expression/activity, or whose upregulated expression/activity, is positively associated with a disease/condition (e.g. a cancer, an infectious disease or an autoimmune disease). The target antigen is preferably expressed at the cell surface of a cell expressing the target antigen.

In some embodiments, the target antigen may be a cancer cell antigen. A cancer cell antigen is an antigen which is expressed or over-expressed by a cancer cell. A cancer cell antigen may be any peptide/polypeptide, glycoprotein, lipoprotein, glycan, glycolipid, lipid, or fragment thereof. A cancer cell antigen's expression may be associated with a cancer. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response. In some embodiments, the antigen is expressed at the cell surface of the cancer cell (i.e. the cancer cell antigen is a cancer cell surface antigen). In some embodiments, the part of the antigen which is bound by the antigen-binding molecule described herein is displayed on the external surface of the cancer cell (i.e. is extracellular). The cancer cell antigen may be a cancer-associated antigen. In some embodiments the cancer cell antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the cancer cell antigen is an antigen whose expression is upregulated (e.g. at the RNA and/or protein level) by cells of a cancer, e.g. as compared to the level of expression of by comparable non-cancerous cells (e.g. non-cancerous cells derived from the same tissue/cell type). In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

Cancer cell antigens are reviewed by Zarour H M, DeLeo A, Finn O J, et al. Categories of Tumor Antigens. In: Kufe D W, Pollock R E, Weichselbaum R R, et al., editors. Holland-Frei Cancer Medicine. 6th edition. Hamilton (ON): B C Decker: 2003. Cancer cell antigens include oncofetal antigens: CEA, Immature laminin receptor, TAG-72; oncoviral antigens such as HPV E6 and E7; overexpressed proteins: BING-4, calcium-activated chloride channel 2, cyclin-B1, 9D7, Ep-CAM, EphA3, HER2/neu, telomerase, mesothelin, SAP-1, surviving cancer-testis antigens: BAGE, CAGE, GAGE, MAGE, SAGE, XAGE, CT9, CT10, NY-ESO-1, PRAME, SSX-2; lineage restricted antigens: MARTI, Gp100, tyrosinase, TRP-1/2, MC1R, prostate specific antigen; mutated antigens: β-catenin, BRCA1/2, CDK4, CML66, Fibronectin, MART-2, p53, Ras, TGF-βRII; post-translationally altered antigens: MUC1, idiotypic antigens: Ig, TCR. Other cancer cell antigens include heat-shock protein 70 (HSP70), heat-shock protein 90 (HSP90), glucose-regulated protein 78 (GRP78), vimentin, nucleolin, feto-acinar pancreatic protein (FAPP), alkaline phosphatase placental-like 2 (ALPPL-2), siglec-5, stress-induced phosphoprotein 1 (STIP1), protein tyrosine kinase 7 (PTK7), and cyclophilin B. In some embodiments the cancer cell antigen is a cancer cell antigen described in Zhao and Cao, Front Immunol. (2019) 10:2250, which is hereby incorporated by reference in its entirety.

In some embodiments, the target antigen is selected from: FAP (fibroblast activation protein), CEA (carcinoembryonic antigen), p95 (p95HER2), BCMA (B-cell maturation antigen), EpCAM (epithelial cell adhesion molecule), MSLN (mesothelin), MCSP (melanoma chondroitin sulfate proteoglycan), HER-1 (human epidermal growth factor 1), HER-2 (human epidermal growth factor 2), HER-3 (human epidermal growth factor 3), CD19, CD20, CD22, CD33, CD38, CD52Flt3, folate receptor 1 (FOLR1), human trophoblast cell-surface antigen 2 (Trop-2) cancer antigen 12-5 (CA-12-5), human leukocyte antigen-antigen D related (HLA-DR), MUC-1 (Mucin-1), A33-antigen, PSMA (prostate-specific membrane antigen), FMS-like tyrosine kinase 3 (FLT-3), PSMA (prostate specific membrane antigen), PSCA (prostate stem cell antigen), transferrin-receptor, TNC (tenascin), carbon anhydrase IX (CA-IX), and/or a peptide bound to a molecule of the human major histocompatibility complex (MHC). In some embodiments, the target antigen is CD19. In some embodiments, the target antigen is FOLR1.

It will be appreciated that the cells and composition of the present disclosure may be used for the treatment/prevention of any disease/condition that would derive therapeutic or prophylactic benefit from a reduction in the level/activity of a given target antigen, or a reduction in the number/proportion/activity of cells comprising/expressing a given target antigen.

For example, the disease/condition may be a disease/condition in which the target antigen, or cells comprising/expressing target antigen are pathologically-implicated, e.g. a disease/condition in which an increased level/activity of the target antigen, or an increase in the number/proportion/activity of cells comprising/expressing target antigen is positively associated with the onset, development or progression of the disease/condition, and/or severity of one or more symptoms of the disease/condition. In some embodiments, an increased level/activity of the target antigen, or an increase in the number/proportion/activity of cells comprising/expressing target antigen may be a risk factor for the onset, development or progression of the disease/condition.

In some embodiments, the disease/condition to be treated/prevented in accordance with the present disclosure is a disease/condition characterized by an increase in the level of expression or activity of the target antigen, e.g. as compared to the level of expression/activity in the absence of the disease/condition. In some embodiments, the disease/condition to be treated/prevented is a disease/condition characterised by an increase in the number/proportion/activity of cells expressing target antigen, e.g. as compared to the level/number/proportion/activity in the absence of the disease/condition (e.g. in a healthy subject, or in equivalent non-diseased tissue). Where the disease/condition is a cancer, the level of expression or activity of the target antigen may be greater than the level of expression or activity of the target antigen in equivalent non-cancerous cells/non-tumor tissue. A cancer/cell thereof may comprise one or more mutations (e.g. relative to equivalent non-cancerous cells/non-tumor tissue) causing upregulation of expression or activity of the target antigen.

Therapeutic/prophylactic intervention in accordance with the present disclosure may achieve one or more of the following in a subject (compared to an equivalent untreated subject, or subject treated with an appropriate control): a reduction in the level of the target antigen; a reduction in the activity of the target antigen; and/or a reduction in the number/proportion/activity of cells comprising/expressing the target antigen.

### Use of Cells and Compositions

In particular, use of the cells and compositions according to the present disclosure in methods to treat/prevent diseases/conditions by adoptive cell transfer (ACT) is contemplated.

Adoptive cell transfer generally refers to a process by which cells (e.g. immune cells) are obtained from a subject, typically by drawing a blood sample from which the cells are isolated. The cells are then typically modified and/or expanded, and then administered either to the same subject (in the case of adoptive transfer of autologous/autogeneic cells) or to a different subject (in the case of adoptive transfer of allogeneic cells). The treatment is typically aimed at providing a population of cells with certain desired characteristics to a subject, or increasing the frequency of such cells with such characteristics in that subject. Adoptive transfer may be performed with the aim of introducing a cell or population of cells into a subject, and/or increasing the frequency of a cell or population of cells in a subject.

Adoptive transfer of immune cells is described, for example, in Kalos and June (2013), Immunity 39 (1): 49-60, and Davis et al. (2015), Cancer J. 21 (6): 486-491, both of which are hereby incorporated by reference in their entirety. The skilled person is able to determine appropriate reagents and procedures for adoptive transfer of cells according to the present disclosure, for example by reference to Dai et al., 2016 J Nat Cancer Inst 108 (7): djv439, which is incorporated by reference in its entirety.

The cells and compositions according to the present disclosure may be employed in the treatment/prevention of diseases/conditions by allotransplantation or autotransplantation.

As used herein. “allotransplantation” refers to the transplantation to a recipient subject of cells, tissues or organs which are genetically non-identical to the recipient subject. The cells, tissues or organs may be from, or may be derived from, cells, tissues or organs of a donor subject that is genetically non-identical to the recipient subject. Allotransplantation is distinct from autotransplantation, which refers to the transplantation of cells, tissues or organs which are from/derived from a donor subject genetically identical to the recipient subject (i.e. autologous material). It will be appreciated that adoptive transfer of allogeneic immune cells is a form of allotransplantation, and that adoptive transfer of autologous immune cells is a form of autotransplantation.

The present disclosure provides methods comprising administering cells and compositions according to the present disclosure to a subject.

In some embodiments, the methods comprise modifying an immune cell to comprise/express polypeptide(s) (e.g. recombinant MAB polypeptide(s)) according to the present disclosure.

In some embodiments, the methods comprise modifying an immune cell to express or comprise a MAB polypeptide according to the present disclosure (e.g. as described herein), and administering the modified immune cell to a subject.

In some embodiments, the methods further comprise administering (i) a recombinant Fc domain-IL2 variant (Fc-IL2v) polypeptide complex comprising a variant Fc domain according to the present disclosure and/or (ii) a targeting antibody comprising a variant Fc domain according to the present disclosure to the subject, wherein the recombinant MAB polypeptide comprises an antigen-binding moiety that binds to the variant Fc domain of the recombinant Fc-IL2v polypeptide complex and/or the targeting antibody.

It will be appreciated that the method steps recited in the preceding three paragraphs may be performed in any suitable order.

In some embodiments, the methods comprise administering to a subject an immune cell modified to express or comprise a recombinant MAB polypeptide, wherein the recombinant MAB polypeptide is a chimeric antigen receptor (CAR) according to the present disclosure.

In some embodiments, the methods comprise administering to a subject an immune cell modified to express or comprise a recombinant MAB polypeptide comprising one or more recombinant CD3-TCR complex polypeptides according to the present disclosure.

In some embodiments, in accordance with the preceding two paragraphs, the subject is a subject to which (i) a recombinant Fc domain-IL2 variant (Fc-IL2v) polypeptide complex comprising a variant Fc domain according to the present disclosure and/or (ii) a targeting antibody comprising a variant Fc domain according to the present disclosure has been administered, or is to be administered, wherein the recombinant MAB polypeptide comprises an antigen-binding moiety that binds to the variant Fc domain.

In some embodiments, the methods comprise:
(a) modifying an immune cell to express or comprise a recombinant MAB polypeptide according to the present disclosure (e.g. as described herein); and
(b) administering (i) a recombinant Fc domain-IL2 variant (Fc-IL2v) polypeptide complex comprising a variant Fc domain according to the present disclosure and/or (ii) a targeting antibody comprising a variant Fc domain according to the present disclosure to a subject; and
(c) administering the modified immune cell to the subject:
wherein the recombinant MAB polypeptide of (a) comprises an antigen-binding moiety that binds to the variant Fc domain of (b).

In some embodiments in accordance with the method of the preceding paragraph, step (c) may be performed before step (b).

In some embodiments, the subject from which the immune cells are isolated/obtained is the same subject to which cells are administered (i.e., adoptive transfer may be of autologous/autogeneic cells). In some embodiments, the subject from which the immune cells are isolated/obtained is a different subject to the subject to which cells are administered (i.e., adoptive transfer may be of allogeneic cells).

In some embodiments, the methods may further comprise one or more of:
(i) obtaining a blood sample from a subject:
(ii) isolating immune cells (e.g. PBMCs) from a blood sample which has been obtained from a subject:
(iii) generating/expanding a population of immune cells:
(iv) culturing the immune cells in in vitro or ex vivo cell culture:
(v) culturing immune cells expressing/comprising a recombinant MAB polypeptide according to the present disclosure in in vitro or ex vivo cell culture:
(vi) collecting/isolating immune cells expressing/comprising a recombinant MAB polypeptide according to the present disclosure according to the present disclosure:
(vii) formulating immune cells expressing/comprising a recombinant MAB polypeptide according to the present disclosure to a pharmaceutical composition, e.g. by mixing the cells with a pharmaceutically-acceptable adjuvant, diluent, or carrier.

Administration of the articles of the present disclosure is preferably in a “therapeutically-effective” or “prophylactically-effective” amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (ed. A. Adejare), 23rd Edition (2020), Academic Press.

Administration of the articles of the present disclosure may be parenteral, systemic, intravenous, intra-arterial, intramuscular, intracavitary, intrathecal, intraocular, intravitreal, intraconjunctival, subretinal, suprachoroidal, subcutaneous, intradermal, intrathecal, oral, nasal, topical or transdermal. Administration may be by injection or infusion. Administration of the articles of the present disclosure may be intratumoral. In some cases, the articles of the present disclosure may be formulated for targeted delivery to specific cells, a tissue, an organ and/or a tumor.

Multiple doses of an article of the present disclosure may be provided. Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months.

Administration of a cell or composition according to the present disclosure to a subject in accordance with the therapeutic and prophylactic intervention described herein may be simultaneous or sequential.

Simultaneous administration refers to administration of (i) a cell or composition according to the present disclosure, and (ii) an antigen-binding molecule described herein together, for example as a pharmaceutical composition containing both agents (i.e. a combined preparation), or immediately after one another, and optionally via the same route of administration, e.g. to the same artery, vein or other blood vessel.

Sequential administration refers to administration of one of (i) a cell or composition according to the present disclosure, and (ii) an antigen-binding molecule described herein, followed after a given time interval by separate administration of the other agent. It is not required that the two agents are administered by the same route, although this is the case in some embodiments. The time interval may be any time interval.

The present disclosure also provides methods for depleting or killing cells comprising or expressing a target antigen, comprising contacting cells comprising/expressing a target antigen with:
(i) an antigen-binding molecule comprising: (a) an antigen-binding domain that binds to the target antigen, and (b) a variant Fc domain according to the present disclosure; and
(ii) an immune cell comprising/expressing a recombinant MAB polypeptide according to the present disclosure:
wherein the MAB polypeptide of (ii) comprises an antigen-binding moiety that binds to the variant Fc domain of the antigen-binding molecule of (i).

### Nucleic Acids and Vectors

The present disclosure provides a nucleic acid, or a plurality of nucleic acids, encoding a recombinant MAB polypeptide, recombinant Fc-IL2v polypeptide complex, or targeting antibody according to the present disclosure. In some embodiments, the nucleic acid(s) comprise or consist of DNA and/or RNA.

A MAB polypeptide, recombinant Fc-IL2v polypeptide complex, or targeting antibody according to the present disclosure may be produced within a cell by translation of RNA encoding the recombinant MAB polypeptide, recombinant Fc-IL2v polypeptide complex, or targeting antibody. A recombinant MAB polypeptide, recombinant Fc-IL2v polypeptide complex, or targeting antibody according to the present disclosure may be produced within a cell by transcription from nucleic acid encoding the recombinant MAB polypeptide, recombinant Fc-IL2v polypeptide complex, or targeting antibody, and subsequent translation of the transcribed RNA.

In some embodiments, the nucleic acid(s) may be, or may be comprised/contained in, a vector, or a plurality of vectors. A “vector” as used herein is a nucleic acid molecule used as a vehicle to transfer exogenous nucleic acid into a cell.

Accordingly, the present disclosure also provides a vector, or plurality of vectors, comprising the nucleic acid or plurality of nucleic acids according to the present disclosure. The vector may facilitate delivery of the nucleic acid(s) encoding a recombinant MAB polypeptide, recombinant Fc-IL2v polypeptide complex, or targeting antibody according to the present disclosure to a cell. The vector may be an expression vector comprising elements required for expressing a recombinant MAB polypeptide, recombinant Fc-IL2v polypeptide complex, or targeting antibody according to the present disclosure. The vector may comprise elements facilitating integration of the nucleic acid(s) into the genomic DNA of cell into which the vector is introduced.

Nucleic acids and vectors according to the present disclosure may be provided in purified or isolated form, i.e. from other nucleic acid, or naturally-occurring biological material.

A vector may be a vector for expression of the nucleic acid in the cell (i.e. an expression vector). Such vectors may include a promoter sequence operably linked to a nucleotide sequence encoding a recombinant MAB polypeptide, recombinant Fc-IL2v polypeptide complex, or targeting antibody according to the present disclosure. A vector may also include a termination codon (i.e. 3′ in the nucleotide sequence of the vector to the nucleotide sequence encoding the recombinant MAB polypeptide, recombinant Fc-IL2v polypeptide complex, or targeting antibody) and expression enhancers. Any suitable vectors, promoters, enhancers and termination codons known in the art may be used to express a peptide or polypeptide from a vector according to the present disclosure.

The term “operably linked” may include the situation where nucleic acid encoding a recombinant MAB polypeptide, recombinant Fc-IL2v polypeptide complex, or targeting antibody according to the present disclosure and regulatory nucleic acid sequence(s) (e.g. a promoter and/or enhancers) are covalently linked in such a way as to place the expression of the nucleic acid encoding a recombinant MAB polypeptide, recombinant Fc-IL2v polypeptide complex, or targeting antibody under the influence or control of the regulatory nucleic acid sequence(s) (thereby forming an expression cassette). Thus, a regulatory sequence is operably linked to the selected nucleic acid sequence if the regulatory sequence is capable of effecting transcription of the nucleic acid sequence. The resulting transcript(s) may then be translated into the desired polypeptide(s).

Vectors contemplated in connection with the present disclosure include DNA vectors. RNA vectors, plasmids (e.g. conjugative plasmids (e.g. F plasmids), non-conjugative plasmids, R plasmids, col plasmids, episomes), viral vectors (e.g. retroviral vectors, e.g. gammaretroviral vectors (e.g. murine Leukemia virus (MLV)-derived vectors, e.g. SFG vector), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 and Morgan and Boyerinas, Biomedicines (2016) 4:9, which are both hereby incorporated by reference in their entirety. In some embodiments, a vector according to the present disclosure is a lentiviral vector.

In some embodiments, the vector may be a eukaryotic vector, i.e. a vector comprising the elements necessary for expression of protein from the vector in a eukaryotic cell. In some embodiments, the vector may be a mammalian vector, e.g. comprising a cytomegalovirus (CMV) or SV40) promoter to drive protein expression.

In some embodiments, a nucleic acid/plurality or vector/plurality according to the present disclosure comprises an EF1α promoter.

In some embodiments, a nucleic acid/plurality or vector/plurality according to the present disclosure encodes a CAR comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to one of SEQ ID NO: 146, SEQ ID NO:149, SEQ ID NO: 151, and SEQ ID NO: 154. In some embodiments, a nucleic acid/plurality or vector/plurality according to the present disclosure encodes a CAR comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:152.

In preferred embodiments, a nucleic acid/plurality or vector/plurality according to the present disclosure comprises the nucleotide sequence of SEQ ID NO: 152, or a codon degenerate nucleotide sequence thereof encoding the amino acid sequence encoded by SEQ ID NO: 152.

In some embodiments, a nucleic acid/plurality or vector/plurality according to the present disclosure encodes a CD3-TCR complex polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to one of SEQ ID NOs: 211 to 255. In some embodiments, a nucleic acid/plurality or vector/plurality according to the present disclosure encodes a CD3-TCR complex polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:222.

In preferred embodiments, a nucleic acid/plurality or vector/plurality according to the present disclosure comprises the nucleotide sequence of SEQ ID NO:287, or a codon degenerate nucleotide sequence thereof encoding the amino acid sequence encoded by SEQ ID NO:287.

As used herein, a “codon degenerate nucleotide sequence” of a reference nucleotide sequence refers to a nucleotide sequence having a non-identical nucleotide sequence to the nucleotide of the reference nucleotide sequence, but encoding the same amino acid sequence as the amino acid sequence encoded by the reference nucleotide sequence, as a consequence of degeneracy of the genetic code.

Constituent polypeptides of a polypeptide complex according to the present disclosure may be encoded by different nucleic acids of a plurality of nucleic acids according to the present disclosure, or by different vectors of a plurality of nucleic acids according to the present disclosure.

In aspects and embodiments of the present disclosure, a nucleic acid, or a plurality of nucleic acids, according to the present disclosure encodes two or more (e.g. 2, 3, 4 or more) recombinant CD3-TCR complex polypeptides according to the present disclosure. In aspects and embodiments of the present disclosure, a vector, or a plurality of vectors, according to the present disclosure encodes two or more (e.g. 2, 3, 4 or more) recombinant CD3-TCR complex polypeptides according to the present disclosure.

In some embodiments in which a nucleic acid/plurality or vector/plurality encodes two or more (e.g. 2, 3, 4 or more) recombinant CD3-TCR complex polypeptides, the recombinant CD3-TCR complex polypeptides are non-identical. In some embodiments, the nucleic acid/plurality or vector/plurality encodes recombinant CD3-TCR complex polypeptides that are complementary. That is, in some embodiments, the nucleic acid/plurality or vector/plurality encodes CD3-TCR complex polypeptides that are capable of associating with one another (e.g. via non-covalent, protein:protein interaction) to form a polypeptide complex (e.g. a polypeptide complex as described herein).

In some embodiments, the nucleic acid/plurality or vector/plurality encodes CD3-TCR complex polypeptides that are capable of associating with one another to form an antigen-binding moiety according to the present disclosure. In some embodiments, the nucleic acid/plurality or vector/plurality encodes CD3-TCR complex polypeptides comprising complementary components of an antigen-binding moiety according to the present disclosure (i.e. components of an antigen-binding moiety that are capable of association (e.g. via non-covalent, protein:protein interaction) to form the antigen-binding moiety).

By way of illustration, in one embodiment a nucleic acid/plurality or vector/plurality encodes (i) a recombinant CD3-TCR complex polypeptide comprising the VH of an antigen-binding moiety specific for a variant Fc domain and the ECD. TMD and ICD of TRAC (T47C), and (ii) a recombinant CD3-TCR complex polypeptide comprising the VL region of the antigen-binding moiety specific for a variant Fc domain, and the ECD. TMD and ICD of TRBC1 (S56C). Following expression from the nucleic acid/plurality or vector/plurality, polypeptides (i) and (ii) associate to form a polypeptide complex comprising an Fv specific for a variant Fc domain, formed by the VH from (i) and the VL from (ii).

In some embodiments, a nucleic acid/plurality or vector/plurality according to the present disclosure encodes:
(i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 146;
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:149;
(iii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:151; or
(iv) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:154.

In some embodiments, a nucleic acid/plurality or vector/plurality according to the present disclosure encodes:
(1) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:227; and
   (ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:255;
or
(2) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:231; and
   (ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:255;
or
(3) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:235; and
   (ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:255;
or
(4) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:239; and
   (ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:243;
or
(5) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:239; and
   (ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:247;
or
(6) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:239; and
   (ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:251.

In preferred embodiments, a nucleic acid/plurality or vector/plurality according to the present disclosure encodes:
(i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:235; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:255.

In preferred embodiments, a nucleic acid/plurality or vector/plurality according to the present disclosure encodes:
(i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:239; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:251.

In preferred embodiments, a nucleic acid/plurality or vector/plurality according to the present disclosure comprises:
(i) the nucleotide sequence of SEQ ID NO:288, or a codon degenerate nucleotide sequence thereof encoding the amino acid sequence encoded by SEQ ID NO:288; and
(i) the nucleotide sequence of SEQ ID NO:289, or a codon degenerate nucleotide sequence thereof encoding the amino acid sequence encoded by SEQ ID NO:289.

In preferred embodiments, a nucleic acid/plurality or vector/plurality according to the present disclosure comprises:
(i) the nucleotide sequence of SEQ ID NO:290, or a codon degenerate nucleotide sequence thereof encoding the amino acid sequence encoded by SEQ ID NO:290; and
(i) the nucleotide sequence of SEQ ID NO:291, or a codon degenerate nucleotide sequence thereof encoding the amino acid sequence encoded by SEQ ID NO:291.

In some embodiments, wherein a nucleic acid/plurality or vector/plurality encodes two or more (e.g. 2, 3, 4 or more) recombinant CD3-TCR complex polypeptides according to the present disclosure, transcription of nucleic acid encoding the two or more recombinant CD3-TCR complex polypeptides is under the control of the same promoter.

In some embodiments, the nucleic acid/plurality or vector/plurality comprises nucleic acid encoding an internal ribosome entry site (IRES). In some embodiments, the IRES is provided in between nucleotide sequences encoding recombinant CD3-TCR complex polypeptides. In some embodiments, the nucleic acid/plurality or vector/plurality comprises nucleic acid permitting the two or more recombinant CD3-TCR complex polypeptides to be translated separately from the same RNA transcript.

In some embodiments, the two or more recombinant CD3-TCR complex polypeptides are encoded by nucleotide sequences provided in the same reading frame. In some embodiments, the nucleic acid/plurality or vector/plurality encodes a fusion protein comprising the two or more recombinant CD3-TCR complex polypeptides. In some embodiments, the fusion protein encoded by the nucleic acid/plurality or vector/plurality comprises a cleavage site (e.g. a cleavage site as described herein) between the amino acid sequences of the recombinant CD3-TCR complex polypeptides. In some embodiments, the nucleic acid/plurality or vector/plurality encodes a composite polypeptide according to the present disclosure.

In some embodiments, transcription of nucleic acid encoding the two or more recombinant CD3-TCR complex polypeptides is under the control of different promoters.

In some embodiments, the nucleic acid/plurality or vector/plurality is multicistronic (e.g. bicistronic, tricistronic, etc.). That is, in some embodiments the nucleic acid/plurality or vector/plurality vector comprises multiple polypeptide-encoding nucleotide sequences. In some embodiments, nucleic acid encoding two or more recombinant CD3-TCR complex polypeptides is provided in different cistrons.

### Cells Comprising/Expressing the Recombinant MAB Polypeptide, Recombinant Fc-IL2v Polypeptide Complex and Nucleic Acids/Vectors of the Disclosure

The present disclosure also provides a cell comprising a recombinant MAB polypeptide, recombinant Fc-IL2v polypeptide complex, or targeting antibody according to the present disclosure, or a nucleic acid/plurality or vector/plurality according to the present disclosure.

It will be appreciated that where cells are referred to herein in the singular (i.e. “a/the cell”), pluralities/populations of such cells are also contemplated.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate). In preferred embodiments, the cell is a human cell.

In some embodiments, the cell is an immune cell. An immune cell may be a cell of hematopoietic origin. e.g. a neutrophil, eosinophil, basophil, dendritic cell, lymphocyte, or monocyte. A lymphocyte may be e.g. a T cell, B cell, NK cell, NKT cell or innate lymphoid cell (ILC), or a precursor thereof. The immune cell may express one or more CD3-TCR complex polypeptides, e.g. TCRα, TCRβ, TCRγ, TCRδ, TRAC, TRBC1, TRBC2, TRGC1, TRGC2, TRDC, CD3ε, CD3δ, CD3γ, CD3ζ and/or CD3η. The immune cell may express CD27, CD28, CD4 and/or CD8. In some embodiments, the immune cell is a T cell, e.g. a CD3+ T cell. In some embodiments, the T cell is a CD3+, CD4+ T cell. In some embodiments, the T cell is a CD3+, CD8+ T cell. In some embodiments, the T cell is a T helper cell (TH cell). In some embodiments, the T cell is a cytotoxic T cell (e.g. a cytotoxic T lymphocyte (CTL)).

Aspects and embodiments of the present disclosure relate particularly to T cells comprising/expressing recombinant MAB polypeptide(s) according to the present disclosure.

In some aspects and embodiments, a cell according to the present disclosure expresses/presents a recombinant MAB polypeptide according to the present disclosure at the cell surface. That is, the recombinant MAB polypeptide may be present in or at the cell membrane. Cells can be evaluated for surface expression of MAB polypeptides (e.g. following introduction in the cell of nucleic acid encoding the same), e.g. using antibody-based methods such as flow cytometry (e.g. as described in Example 1 of the present disclosure).

In aspects and embodiments of the present disclosure, a cell according to the present disclosure comprises or expresses a recombinant MAB polypeptide according to the present disclosure. In some aspects and embodiments, a cell according to the present disclosure comprises nucleic acid encoding a recombinant MAB polypeptide according to the present disclosure. In some aspects and embodiments, a cell according to the present disclosure comprises a nucleic acid/plurality or vector/plurality according to the present disclosure.

In aspects and embodiments of the present disclosure, a cell according to the present disclosure comprises or expresses a recombinant MAB polypeptide according to the present disclosure that binds to a variant Fc domain as described herein. It will be appreciated that binding to the variant Fc domain is achieved though the binding moiety of the recombinant MAB polypeptide(s). The cell may express/comprise a recombinant MAB polypeptide according to the present disclosure as a consequence of expression of nucleic acid encoding such recombinant MAB polypeptide. The cell may have been engineered to comprise nucleic acid encoding such a recombinant MAB polypeptide.

In some embodiments, a cell according to the present disclosure may comprise modification to reduce expression of a CD3-TCR complex polypeptide (i.e. as compared to the level of expression of the CD3-TCR complex polypeptide by an equivalent unmodified cell). In some embodiments, the cell comprises modification to reduce expression of an endogenous CD3-TCR complex polypeptide, i.e. a CD3-TCR complex polypeptide encoded by the genome of an equivalent unmodified cell.

In some embodiments, the cell comprises modification to reduce expression of the CD3-TCR complex polypeptide from which the CD3-TCR complex association domain of the recombinant polypeptide CD3-TCR complex polypeptide is derived. By way of illustration, in embodiments wherein the cell comprises or expresses a recombinant CD3-TCR complex polypeptide comprising a CD3-TCR complex association domain derived from CD3ε (or a composite polypeptide or polypeptide complex comprising such a recombinant CD3-TCR complex polypeptide), the cell may comprise modification to reduce expression by the cell of CD3ε (i.e. endogenous CD38). By way of further illustration, in embodiments wherein the cell comprises or expresses recombinant CD3-TCR complex polypeptide(s) comprising a CD3-TCR complex association domain(s) derived from TRAC. TRBC1 and/or TRBC2 (or a composite polypeptide or polypeptide complex comprising such recombinant CD3-TCR complex polypeptide(s)), the cell may comprise modification to reduce expression by the cell of TRAC/TRBC1/TRBC2 (i.e. endogenous TRAC/TRBC1/TRBC2).

In some embodiments, the cell comprises modification to nucleic acid (e.g. endogenous nucleic acid) encoding the CD3-TCR complex polypeptide. In some embodiments, one or more alleles of the gene encoding the CD3-TCR complex polypeptide in the cell are modified. In some embodiments, the modification comprises an insertion, substitution or deletion in the nucleotide sequence of nucleic acid encoding the CD3-TCR complex polypeptide. In some embodiments, the modification reduces or prevents the endogenous expression of the CD3-TCR complex polypeptide from the modified nucleotide sequence. In some embodiments, the modified cell lacks endogenous nucleic acid encoding the CD3-TCR complex polypeptide. In some embodiments, the modification introduces a premature stop codon in the nucleotide sequence of RNA transcribed from endogenous nucleic acid encoding the CD3-TCR complex polypeptide. In some embodiments, the nucleotide sequence of the modified nucleic acid encodes a truncated and/or non-functional version of the CD3-TCR complex polypeptide. In some embodiments, the nucleotide sequence of the modified nucleic acid encodes a version of the CD3-TCR complex polypeptide that is misfolded and/or degraded. In some embodiments, the nucleotide sequence of the modified nucleic acid encodes a version of the CD3-TCR complex polypeptide that is incapable of participating in a functional CD3-TCR polypeptide complex.

In some embodiments, the cell comprises modification to nucleic acid (e.g. endogenous nucleic acid) encoding CD3ε (e.g. the polypeptide having the sequence of SEQ ID NO:181). In some embodiments, one or more alleles of CD3E are modified. In some embodiments, the modification comprises an insertion, substitution or deletion in the nucleotide sequence of CD3E. In some embodiments, the modification reduces or prevents the endogenous expression of CD3ε by the cell. In some embodiments, the modified cell lacks endogenous nucleic acid encoding CD3ε. In some embodiments, the modification introduces a premature stop codon in the nucleotide sequence of RNA transcribed from endogenous nucleic acid encoding CD3ε. In some embodiments, the nucleotide sequence of the modified nucleic acid encodes a truncated and/or non-functional version of CD3ε. In some embodiments, the nucleotide sequence of the modified nucleic acid encodes a version of CD3ε that is misfolded and/or degraded. In some embodiments, the nucleotide sequence of the modified nucleic acid encodes a version of CD3ε that is incapable of participating in a functional CD3-TCR polypeptide complex.

In some embodiments, the cell comprises modification to nucleic acid (e.g. endogenous nucleic acid) encoding TRAC (e.g. the polypeptide having the sequence of SEQ ID NO: 157). TRBC1 (e.g. the polypeptide having the sequence of SEQ ID NO: 161) and/or TRBC2 (e.g. the polypeptide having the sequence of SEQ ID NO:165). In some embodiments, the cell comprises modification to nucleic acid (e.g. endogenous nucleic acid) encoding TRAC and TRBC1. In some embodiments, one or more alleles of TRAC, TRBC1 and/or TRBC2 (e.g. TRAC and TRBC1) are modified. In some embodiments, the modification comprises an insertion, substitution or deletion in the nucleotide sequence of TRAC, TRBC1 and/or TRBC2 (e.g. TRAC and TRBC1). In some embodiments, the modification reduces or prevents the endogenous expression of TRAC, TRBC1 and/or TRBC2 (e.g. TRAC and TRBC1) by the cell. In some embodiments, the modified cell lacks endogenous nucleic acid encoding TRAC, TRBC1 and/or TRBC2 (e.g. TRAC and TRBC1). In some embodiments, the modification introduces a premature stop codon in the nucleotide sequence of RNA transcribed from endogenous nucleic acid encoding TRAC, TRBC1 and/or TRBC2 (e.g. TRAC and TRBC1). In some embodiments, the nucleotide sequence of the modified nucleic acid encodes a truncated and/or non-functional version of TRAC, TRBC1 and/or TRBC2 (e.g. TRAC and TRBC1). In some embodiments, the nucleotide sequence of the modified nucleic acid encodes a version of TRAC, TRBC1 and/or TRBC2 (e.g. TRAC and TRBC1) that is misfolded and/or degraded. In some embodiments, the nucleotide sequence of the modified nucleic acid encodes a version of TRAC, TRBC1 and/or TRBC2 (e.g. TRAC and TRBC1) that is incapable of participating in a functional CD3-TCR polypeptide complex.

An immune cell (e.g. a T cell) according to the present disclosure may be characterized by certain functional properties in response to the antigen for which the recombinant MAB polypeptide comprises an antigen-binding moiety (or in response to a cell comprising or expressing the antigen): CD3-TCR complex-mediated signalling; cell proliferation/population expansion, growth factor (e.g. IL-2) expression, IFNγ expression, CD107a expression, TNFα expression, GM-CSF expression, perforin expression, granzyme expression, granulysin expression, and/or FAS ligand (FASL) expression.

For example, an immune cell (e.g. a T cell) according to the present disclosure may display one or more of the functional properties recited in the preceding paragraph in response to a variant Fc domain according to the present disclosure (i.e. a variant Fc domain that is bound by antigen-binding moiety comprised in a recombinant MAB polypeptide expressed by the cell), or in response to a cell comprising/expressing such a variant Fc domain.

CD3-TCR complex-mediated signalling can be investigated by analysing one or more correlates of CD3-TCR complex-mediated signalling. For example, CD3-TCR complex-mediated signalling can be investigated by evaluating phosphorylation of one or more signal transduction molecules of CD3-TCR complex signalling pathway. The level of CD3-TCR complex-mediated signalling can be analysed by detection and quantification of the level of phosphorylation of CD3ζ, ZAP-70, Lck, LAT and/or SLP-76. The level of CD3-TCR complex-mediated signalling can also be analysed using reporter-based methods. e.g. methods quantifying the activity of a transcription factor whose expression/activity is upregulated in response to signalling through the CD3-TCR complex, e.g. NFAT, NF-κB and/or AP-1, or through methods quantifying expression of a gene whose expression is upregulated by signalling through the CD3-TCR complex, e.g. IL2. For example, CD3-TCR complex-mediated signalling can be investigated using a reporter cell line stably expressing a luciferase reporter driven by CD3-TCR complex-mediated signalling (e.g. GloResponse Jurkat NFAT-RE-luc2P (Promega #CS176501) or T Cell Activation Bioassay TCRαβ-KO CD4+ (Promega #GA1172), as in Example 1 of the present disclosure.

Cell proliferation/population expansion can be investigated by analysing cell division or the number of cells over a period of time. Cell division can be analysed, for example, by in vitro analysis of incorporation of ³H-thymidine or by CFSE dilution assay, e.g. as described in Fulcher and Wong. Immunol Cell Biol (1999) 77 (6): 559-564, hereby incorporated by reference in entirety. Proliferating cells can also be identified by analysis of incorporation of 5-ethynyl-2′-deoxyuridine (EdU), as described e.g. in Buck et al., Biotechniques. 2008 June: 44 (7): 927-9, and Sali and Mitchison, PNAS USA 2008 Feb. 19; 105 (7): 2415-2420, both hereby incorporated by reference in their entirety.

As used herein. “expression” may be gene or protein expression. Gene expression encompasses transcription of DNA to RNA, and can be measured by various means known to those skilled in the art, for example by measuring levels of mRNA by quantitative real-time PCR (qRT-PCR), or using reporter-based methods. Similarly, protein expression can be measured by various methods well known in the art. e.g. antibody-based methods, for example by western blot, immunohistochemistry, immunocytochemistry, flow cytometry, ELISA, ELISPOT, or reporter-based methods.

An immune cell (e.g. a T cell) according to the present disclosure may display cytotoxicity to cells comprising/expressing a variant Fc domain according to the present disclosure. That is, an immune cell (e.g. a T cell) according to the present disclosure may posses the ability to kill cells comprising/expressing a variant Fc domain according to the present disclosure.

A cell comprising a variant Fc domain according to the present disclosure may do so as a consequence of binding of an antigen-binding molecule comprising the variant Fc domain to an antigen expressed by the cell (e.g. at the surface of the cell, i.e. in or at the cell membrane). In some embodiments, a cell comprising a variant Fc domain according to the present disclosure comprises (e.g. at the surface of the cell) a polypeptide complex comprising (i) an antigen-binding molecule comprising the variant Fc domain, and (ii) the target antigen for the antigen-binding molecule.

Cytotoxicity and cell killing can be investigated, for example, using any of the methods reviewed in Zaritskaya et al., Expert Rev Vaccines (2011), 9 (6): 601-616, hereby incorporated by reference in its entirety. Examples of in vitro assays of cytotoxicity/cell killing assays include release assays such as the ⁵¹Cr release assay, the lactate dehydrogenase (LDH) release assay, the 3-(4,5-dimethylthiazol-2-yl)-2,5-diphenyl tetrazolium bromide (MTT) release assay, and the calcein-acetoxymethyl (calcein-AM) release assay. These assays measure cell killing based on the detection of factors released from lysed cells. Cell killing by a given test cell type (e.g. an immune cell (e.g. a T cell) according to the present disclosure) can be analysed e.g. by co-culturing the test cells with the given target cell type (e.g. a cell comprising a variant Fc domain according to the present disclosure), and measuring the number/proportion of viable (i.e. non-lysed)/dead (e.g. lysed) target cells after a suitable period of time. Other suitable assays include the xCELLigence real-time cytolytic in vitro potency assay described in Cerignoli et al., PLOS One. (2018) 13 (3): e0193498 (hereby incorporated by reference in its entirety), and the Incucyte immune cell killing assay, which is employed in the experimental examples of the present disclosure.

An immune cell (e.g. a T cell) according to the present disclosure may possess one or more novel, similar or improved functional properties as compared to a CAR or TCR comprising the same antigen-binding moiety (i.e. the antigen-binding moiety of the recombinant MAB polypeptide expressed by the cell). In some embodiments, an immune cell according to the present disclosure may possess one or more novel, similar or improved functional properties as compared to a CAR-expressing cell described in WO 2018/177966 A1, which is hereby incorporated by reference in its entirety.

In some embodiments, an immune cell (e.g. a T cell) according to the present disclosure may display a level of CD3-TCR complex-mediated signalling in response to the antigen for which CD3-TCR polypeptide complex comprises an antigen-binding moiety, or in response to a cell comprising or expressing the antigen (e.g. in response to a variant Fc domain according to the present disclosure (i.e. a variant Fc domain that is bound by antigen-binding moiety comprised in a recombinant MAB polypeptide expressed by the cell), or in response to a cell comprising/expressing such a variant Fc domain) that is similar to, or greater than the level of CD3-TCR complex-mediated signalling displayed by a CAR comprising the same antigen-binding moiety.

A level of CD3-TCR complex-mediated signalling which is “similar to” a reference level of CD3-TCR complex-mediated signalling may be ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times, ≥0.95 times and ≤1.1 times the reference level of CD3-TCR complex-mediated signalling. In some embodiments, a level of CD3-TCR complex-mediated signalling which is “greater than” a reference level of CD3-TCR complex-mediated signalling may be greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, 23 times, 24 times or ≥5 times the reference level of CD3-TCR complex-mediated signalling.

In some embodiments, a T cell expressing a recombinant MAB polypeptide according to the present disclosure comprising an antigen-binding moiety comprising a VH region according to SEQ ID NO:20 and a VL region according to SEQ ID NO:23 may be evaluated in an assay comprising:
(i) contacting T cells expressing a recombinant MAB polypeptide according to the present disclosure comprising an antigen-binding moiety comprising a VH region according to SEQ ID NO:20 and a VL region according to SEQ ID NO:23 with antigen-presenting cells, which are cells expressing a given target antigen that have been contacted with a targeting antibody according to the present disclosure, and which comprises an Fc domain having a CH2-CH3 region according to SEQ ID NO:7, and subsequently analysing the level of CD3-TCR complex-mediated signalling by the T cells;
(ii) contacting T cells (e.g. equivalent T cells, i.e. derived from the same source as the T cells of (i)) expressing a CAR according to SEQ ID NO: 146, with antigen-presenting cells as defined in (i), and subsequently analysing the level of CD3-TCR complex-mediated signalling by the T cells; and
(iii) comparing the level of CD3-TCR complex-mediated signalling by the T cells of (i) with the T cells of (ii).

In some embodiments, in an assay performed as described in the preceding paragraph, T cells according to (i) display a level of CD3-TCR complex-mediated signalling which is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times, ≥0.95 times and ≤1.1 times the level of CD3-TCR complex-mediated signalling displayed by T cells according to (ii). In some embodiments, in an assay performed as described in the preceding paragraph, T cells according to (i) display a level of CD3-TCR complex-mediated signalling which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times or ≥5 times the level of CD3-TCR complex-mediated signalling displayed by T cells according to (ii).

The present disclosure also provides methods for producing a cell according to the present disclosure, and the cells obtained or obtainable by such methods.

Methods for producing cells comprising/expressing a polypeptide/polypeptide complex of interest are well known to the skilled person, and generally comprise introducing nucleic acid(s)/vector(s) encoding the polypeptide(s) of interest into the cells.

Such methods may comprise nucleic acid transfer for permanent (i.e. stable) or transient expression of the transferred nucleic acid. In some embodiments, following introduction into a cell nucleic acid(s) encoding the polypeptide(s) of interest may be integrated into or form part of the genomic DNA of the cell. In some embodiments, following introduction into a cell nucleic acid(s) encoding the polypeptide(s) of interest may be maintained extrachromosomally.

Any suitable genetic engineering platform may be used, and include gammaretroviral vectors, lentiviral vectors, adenovirus vectors. DNA transfection, transposon-based gene delivery and RNA transfection, for example as described in Maus et al., Annu Rev Immunol (2014) 32:189-225, hereby incorporated by reference in its entirety. Methods also include those described e.g. in Wang and Rivière Mol Ther Oncolytics. (2016) 3:16015, which is hereby incorporated by reference in its entirety. Suitable methods for introducing nucleic acid(s)/vector(s) into cells include transduction, transfection and electroporation.

Methods for generating/expanding populations of cells comprising/expressing polypeptide(s) of interest in vitrolex vivo are well known to the skilled person. Suitable culture conditions (i.e. cell culture media, additives, stimulations, temperature, gaseous atmosphere), cell numbers, culture periods and methods for introducing nucleic acid(s)/vector(s) encoding polypeptide(s) of interest into cells, etc. can be determined by reference e.g. to WO 2018/177966 A1. In some embodiments, a cell/population of cells according to the present disclosure is prepared under GMP (good manufacturing practice; e.g. as described in the guidelines for good manufacturing practice published by the European Commission (Volume 4 of ‘The rules governing medicinal products in the European Union’ contains guidance for the interpretation of the principles and guidelines of good manufacturing practices for medicinal products for human and veterinary use laid down in Commission Directives 91/356/EEC, as amended by Directive 2003/94/EC, and 91/412/EEC respectively) conditions.

Conveniently, cultures of cells according to the present disclosure may be maintained at 37° C., in a humidified atmosphere containing 5% CO₂. The cells of cell cultures can be established and/or maintained at any suitable density, as can readily be determined by the skilled person. Cultures can be performed in any vessel suitable for the volume of the culture, e.g. in wells of a cell culture plate, cell culture flasks, a bioreactor, etc. In some embodiments cells are cultured in a bioreactor, e.g. a bioreactor described in Somerville and Dudley, Oncoimmunology (2012) 1(8):1435-1437, which is hereby incorporated by reference in its entirety. Immune cells (e.g. T cells) may be activated prior to introduction of nucleic acid(s) encoding the polypeptide(s) of interest. For example, T cells within a population of PBMCs may be non-specifically activated by stimulation in vitro with agonist anti-CD3 and agonist anti-CD28 antibodies, in the presence of IL-2.

Introducing nucleic acid(s) into a cell may comprise transduction, e.g. lentiviral transduction. Transduction of immune cells with viral vectors is described e.g. in Simmons and Alberola-Ila, Methods Mol Biol. (2016) 1323:99-108, which is hereby incorporated by reference in its entirety.

Agents may be employed to enhance the efficiency of transduction. Hexadimethrine bromide (polybrene) is a cationic polymer which is commonly used to improve transduction, through neutralising charge repulsion between virions and sialic acid residues expressed on the cell surface. Other agents commonly used to enhance transduction include e.g. the poloxamer-based agents such as LentiBOOST (Sirion Biotech). Retronectin (Takara). Vectofusin (Miltenyi Biotech) and also SureENTRY (Qiagen) and ViraDuctin (Cell Biolabs). In some embodiments the methods comprise centrifuging the cells into which it is desired to introduce nucleic acid encoding the polypeptide(s) of interest in the presence of cell culture medium comprising viral vector comprising the nucleic acid (referred to in the art as “spinfection”).

The methods generally comprise introducing a nucleic acid encoding polypeptide(s) of interest into a cell, and culturing the cell under conditions suitable for expression of the polypeptide(s) of interest by the cell. In some embodiments, the methods comprise culturing immune cells into which nucleic acid encoding polypeptide(s) of interest has been introduced in order to expand their number.

In some embodiments, the methods comprise analysing the cells to confirm successful introduction of the nucleic acid into the cells. In some embodiments, the methods comprise analysing the cells to confirm expression of the polypeptide(s) of interest by the cells (e.g. via evaluation of a detectable entity).

In some embodiments the methods further comprise cells expressing the polypeptide(s) of interest, e.g. from other cells (e.g. cells which do not express the polypeptide(s) of interest). Methods for purifying/isolating immune cells from heterogeneous populations of cells are well known in the art, and may employ e.g. FACS- or MACS-based methods for sorting populations of cells based on the expression of markers of the immune cells. In some embodiments the methods purifying/isolating cells of a particular type, e.g. CD8+ T cells or CTLs expressing the polypeptide(s) of interest.

Methods for producing cells according to the present disclosure may comprise modifying the cells to reduce the expression of a CD3-TCR complex polypeptide. In some embodiments, the methods comprise modifying nucleic acid (e.g. endogenous nucleic acid) encoding the CD3-TCR complex polypeptide.

Modification of a given target nucleic acid can be achieved in a variety of ways known to the skilled person, including modification of the target nucleic acid by homologous recombination, and target nucleic acid editing using site-specific nucleases (SSNs).

Suitable methods may employ targeting by homologous recombination, which is reviewed, for example, in Mortensen Curr Protoc Neurosci. (2007) Chapter 4: Unit 4.29 and Vasquez et al., PNAS 2001, 98 (15): 8403-8410 both of which are hereby incorporated by reference in their entirety. Targeting by homologous recombination involves the exchange of nucleic acid sequence through crossover events guided by homologous sequences. Other suitable techniques include nucleic acid editing using SSNs. Gene editing using SSNs is reviewed e.g. in Eid and Mahfouz, Exp Mol Med. 2016 October: 48 (10): e265, which is hereby incorporated by reference in its entirety. Enzymes capable of creating site-specific double strand breaks (DSBs) can be engineered to introduce DSBs to target nucleic acid sequence(s) of interest. DSBs may be repaired by either error-prone non-homologous end-joining (NHEJ), in which the two ends of the break are rejoined, often with insertion or deletion of nucleotides. Alternatively. DSBs may be repaired by homology-directed repair (HDR), a high-fidelity mechanism in which a DNA template with ends homologous to the break site is supplied and introduced at the site of the DSB.

SSNs capable of being engineered to generate target nucleic acid sequence-specific DSBs include zinc-finger nucleases (ZFNs), transcription activator-like effector nucleases (TALENs) and clustered regularly interspaced palindromic repeats/CRISPR-associated-9 (CRISPR/Cas9) systems. ZFN systems are reviewed e.g. in Umov et al., Nat Rev Genet. (2010) 11 (9): 636-46, which is hereby incorporated by reference in its entirety. ZFNs comprise a programmable Zinc Finger DNA-binding domain and a DNA-cleaving domain (e.g. a FokI endonuclease domain). The DNA-binding domain may be identified by screening a Zinc Finger array capable of binding to the target nucleic acid sequence. TALEN systems are reviewed e.g. in Mahfouz et al., Plant Biotechnol J. (2014) 12 (8): 1006-14, which is hereby incorporated by reference in its entirety. TALENs comprise a programmable DNA-binding TALE domain and a DNA-cleaving domain (e.g. a FokI endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: ‘HD’ binds to C, ‘NI’ binds to A, ‘NG’ binds to T and ‘NN’ or ‘NK’ binds to G (Moscou and Bogdanove, Science (2009) 326 (5959): 1501.). CRISPR/Cas9 and related systems e.g. CRISPR/Cpf1, CRISPR/C2c1, CRISPR/C2c2 and CRISPR/C2c3 are reviewed e.g. in Nakade et al., Bioengineered (2017) 8(3):265-273, which is hereby incorporated by reference in its entirety. These systems comprise an endonuclease (e.g. Cas9, Cpf1 etc.) and the single-guide RNA (sgRNA) molecule. The sgRNA can be engineered to target endonuclease activity to nucleic acid sequences of interest.

In some embodiments, modifying nucleic acid (e.g. endogenous nucleic acid) encoding the CD3-TCR complex polypeptide in accordance with the present disclosure employs a site-specific nuclease (SSN) system targeting nucleic acid encoding the CD3-TCR complex polypeptide. The SSN system may be a ZFN system, a TALEN system, CRISPR/Cas9 system, a CRISPR/Cpf1 system, a CRISPR/C2c1 system, a CRISPR/C2c2 system or a CRISPR/C2c3 system.

In some embodiments, a method for producing a cell according to the present disclosure comprises introducing nucleic acid(s) encoding a CRISPR/Cas9 system targeting CD3E into a cell. In some embodiments, the nucleic acid(s) encode a CRISPR RNA (crRNA) targeting CD3E (e.g. an exon of CD3E, e.g. exon 7 of CD3E) and a trans-activating crRNA (tracrRNA) for processing the crRNA to its mature form.

In some embodiments, a method for producing a cell according to the present disclosure comprises introducing nucleic acid(s) encoding CRISPR/Cas9 system(s) targeting TRAC, TRBC1 and/or TRBC2 (e.g. TRAC and TRBC1) into a cell. In some embodiments, the nucleic acid(s) encode a CRISPR RNA (crRNA) targeting TRAC, TRBC1 and/or TRBC2 (e.g. TRAC and TRBC1; e.g. an exon of TRAC, TRBC1 and/or TRBC2 (e.g. TRAC and TRBC1)) and a trans-activating crRNA (tracrRNA) for processing the crRNA to its mature form.

### Compositions

The present disclosure also provides compositions comprising the recombinant MAB polypeptide, recombinant Fc-IL2v polypeptide complex, nucleic acids, expression vectors and cells described herein.

The recombinant MAB polypeptide, recombinant Fc-IL2v polypeptide complex, nucleic acids, expression vectors and cells described herein (and particularly the nucleic acids, expression vectors and cells described herein) may be formulated as pharmaceutical compositions or medicaments for clinical use and may comprise a pharmaceutically-acceptable carrier, diluent, excipient or adjuvant. In preferred aspects and embodiments, the present disclosure provides a pharmaceutical composition or medicament comprising a cell according to the present disclosure. Thus, the present disclosure also provides a pharmaceutical composition/medicament comprising a polypeptide, polypeptide complex, nucleic acid/plurality, expression vector/plurality or cell described herein. In preferred embodiments, a pharmaceutical composition/medicament according to the present disclosure comprises a nucleic acid/plurality, expression vector/plurality or cell described herein.

The pharmaceutical compositions/medicaments of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term “pharmaceutically-acceptable” as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be “acceptable” in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23rd Edition (2020), Academic Press.

Pharmaceutical compositions and medicaments of the present disclosure may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration. In some embodiments, a pharmaceutical composition/medicament may be formulated for administration by injection or infusion, or administration by ingestion.

Suitable formulations may comprise the cell provided in a sterile or isotonic medium. Medicaments and pharmaceutical compositions may be formulated in fluid, including gel form. Fluid formulations may be formulated for administration by injection or infusion (e.g. via catheter) to a selected region of the human or animal body.

In some embodiments, the pharmaceutical compositions/medicament is formulated for injection or infusion, e.g. into a blood vessel, tissue/organ of interest, or a tumor.

The present disclosure also provides methods for the production of pharmaceutically useful compositions, such methods of production may comprise one or more steps selected from:
(i) producing a cell described herein;
(ii) isolating/purifying a cell described herein; and/or
(iii) mixing a cell described herein with a pharmaceutically-acceptable carrier, adjuvant, excipient or diluent.

For example, a further aspect the present disclosure relates to a method of formulating or producing a medicament or pharmaceutical composition for use in the treatment of a disease/condition (e.g. a disease/condition described herein), the method comprising formulating a pharmaceutical composition or medicament by mixing a cell described herein with a pharmaceutically-acceptable carrier, adjuvant, excipient or diluent.

### Subjects

A subject in accordance with the various aspects of the present disclosure may be any animal or human. Therapeutic and prophylactic applications may be in human or animals (veterinary use).

The subject to be administered with an article of the present disclosure (e.g. in accordance with therapeutic or prophylactic intervention) may be a subject in need of such intervention. The subject is preferably mammalian, more preferably human. The subject may be a non-human mammal, but is more preferably human. The subject may be male or female. The subject may be a patient.

A subject may have (e.g. may have been diagnosed with) a disease or condition described herein, may be suspected of having such a disease/condition, or may be at risk of developing/contracting such a disease/condition. In embodiments according to the present disclosure, a subject may be selected for treatment according to the methods based on characterisation for one or more markers of such a disease/condition.

In some embodiments, a subject may be selected for therapeutic or prophylactic intervention as described herein based on the detection of cells/tissue expressing a target antigen (i.e. the target antigen of an antigen-binding molecule to be employed in conjunction with a cell or composition according to the present disclosure), or of cells/tissue overexpressing the target antigen, e.g. in a sample obtained from the subject.

A subject may be an allogeneic or non-autologous subject with respect to an intervention in accordance with the present disclosure. As used herein, where a subject is referred to herein as being “allogeneic” or “non-autologous” with respect to an intervention, the subject is a subject other than the subject from which the cell of the intervention (i.e. the cell to be administered, or the cell of the pharmaceutical composition/medicament to be administered) is derived. A subject to be treated/prevented in accordance with the present disclosure may be genetically non-identical to the subject from which the cell (e.g. the cell of the pharmaceutical composition/medicament) to be administered to the subject is derived. A subject to be treated/prevented in accordance with the present disclosure may comprise MHC/HLA genes encoding MHC/HLA molecules (e.g. MHC class I a and/or MHC class II molecules) that are non-identical to the MHC/HLA molecules (e.g. MHC class I a and/or MHC class II molecules) encoded by the cell (e.g. the cell of the pharmaceutical composition/medicament) to be administered to the subject. A subject to be treated/prevented in accordance with the present disclosure may be HLA-mismatched with respect to the subject from which the cell (e.g. the cell of the pharmaceutical composition/medicament) to be administered to the subject is derived.

The subject to which cells are administered in accordance with the present disclosure may be allogeneic/non-autologous with respect to the source from which the cell (e.g. the cell of the pharmaceutical composition/medicament) to be administered to the subject is derived. The subject to which cells are administered may be a different subject to the subject from which cells are/were obtained for the production of the cell (e.g. the cell of the pharmaceutical composition/medicament) to be administered to the subject. The subject to which the cell is administered may be genetically non-identical to the subject from which the cell (e.g. the cell of the pharmaceutical composition/medicament) to be administered to the subject cells are/were obtained for the production of the cells.

A subject may be an autogeneic/autologous subject with respect to an intervention in accordance with the present disclosure. As used herein, where a subject is referred to herein as being “autogeneic” or “autologous” with respect to an intervention, the subject is the same subject from which the cell of the intervention (i.e. the cell to be administered, or the cell of the pharmaceutical composition/medicament to be administered) is derived. A subject to be treated/prevented in accordance with the present disclosure may be genetically identical to the subject from which the cell (e.g. the cell of the pharmaceutical composition/medicament) to be administered to the subject is derived. A subject to be treated/prevented in accordance with the present disclosure may comprise MHC/HLA genes encoding MHC/HLA molecules (e.g. MHC class I a and/or MHC class II molecules) that are identical to the MHC/HLA molecules (e.g. MHC class I a and/or MHC class II molecules) encoded by the cell (e.g. the cell of the pharmaceutical composition/medicament) to be administered to the subject. A subject to be treated/prevented in accordance with the present disclosure may be HLA-matched with respect to the subject from which the cell (e.g. the cell of the pharmaceutical composition/medicament) to be administered to the subject is derived.

The subject to which cells are administered in accordance with the present disclosure may be autogeneic/autologous with respect to the source from which the cell (e.g. the cell of the pharmaceutical composition/medicament) to be administered to the subject is derived. The subject to which cells are administered may be the same subject as the subject from which cells are/were obtained for the production of the cell (e.g. the cell of the pharmaceutical composition/medicament) to be administered to the subject. The subject to which the cell is administered may be genetically identical to the subject from which the cell (e.g. the cell of the pharmaceutical composition/medicament) to be administered to the subject cells are/were obtained for the production of the cells.

### Kits

The present disclosure also provides kits of parts.

In some aspects and embodiments, a kit of parts according to the present disclosure comprises (i) a cell according to the present disclosure, (ii) a recombinant Fc-IL2v polypeptide complex comprising a variant Fc domain according to the present disclosure, and (iii) a targeting antibody comprising a variant Fc domain according to the present disclosure. It will be appreciated that in accordance with such aspects and embodiments, the cell of (i) comprises/expresses a recombinant MAB polypeptide according to the present disclosure comprising an antigen-binding moiety that binds to the variant Fc domain of (ii) and (iii).

In some aspects and embodiments, a kit of parts according to the present disclosure comprises (i) a composition according to the present disclosure, and (ii) a recombinant Fc-IL2v polypeptide complex comprising a variant Fc domain according to the present disclosure, and (iii) a targeting antibody comprising a variant Fc domain according to the present disclosure. It will be appreciated that in accordance with such aspects and embodiments, the composition of (i) comprises a cell comprising/expressing a recombinant MAB polypeptide according to the present disclosure comprising an antigen-binding moiety that binds to the variant Fc domain (ii) and (iii).

In some aspects and embodiments, a kit of parts according to the present disclosure comprises (i) a nucleic acid/plurality or an expression vector/plurality according to the present disclosure, and (ii) a recombinant Fc-IL2v polypeptide complex comprising a variant Fc domain according to the present disclosure, and (iii) a targeting antibody comprising a variant Fc domain according to the present disclosure. It will be appreciated that in accordance with such aspects and embodiments, the nucleic acid/plurality or expression vector/plurality of (i) encode polypeptide(s) for engineering a cell to comprise/express a MAB polypeptide according to the present disclosure comprising an antigen-binding moiety that binds to the variant Fc domain of (ii) and (iii).

Kits of parts according to the present disclosure may comprise a predetermined quantity of articles according to (i), (ii), and/or (iii), as described in the preceding three paragraphs. In some embodiments, articles according to (i), (ii), and/or (iii) are provided in containers (e.g. in vials or bottles). The kit may provide articles according to (i), (ii), and/or (iii) together with instructions (e.g. a protocol) as to how to employ them in accordance with a therapeutic or prophylactic intervention as described herein.

In some embodiments, a kit of parts comprises materials for producing a polypeptide according to the present disclosure, e.g. a recombinant MAB polypeptide according to the present disclosure. In some embodiments, a kit of parts comprises materials for producing a polypeptide complex according to the present disclosure, e.g. a recombinant MAB polypeptide comprising a recombinant CD3-TCR complex polypeptide according to the present disclosure. In some embodiments, a kit of parts comprises materials for producing a cell according to the present disclosure, e.g. recombinant MAB polypeptide comprising a chimeric antigen receptor according to the present disclosure. In some embodiments, a kit of parts comprises materials for producing a composition according to the present disclosure, e.g. a pharmaceutical composition comprising a cell according to the present disclosure, e.g. a cell comprising/expressing a recombinant MAB polypeptide according to the present disclosure.

In some embodiments, the kit of parts may comprise a nucleic acid/plurality or an expression vector/plurality according to the present disclosure, and optionally materials for introducing the nucleic acid/plurality or an expression vector/plurality into a cell. In some embodiments, the kit of parts may comprise a system for producing a cell according to the present disclosure in accordance with GMP conditions. In some embodiments, the kit of parts may comprise a (closed) bag cell incubation system in which a nucleic acid/plurality or an expression vector/plurality according to the present disclosure can be introduced into a cell and subsequently cultured under GMP conditions.

In some embodiments, the kit of parts may comprise materials for formulating a cell according to the present disclosure to a pharmaceutical composition, e.g. a pharmaceutically-acceptable carrier, diluent, excipient or adjuvant.

The manufacture of kits of parts according to the present disclosure preferably follows standard procedures which are known to the person skilled in the art.

### Sequence Identity

As used herein, “sequence identity” refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780) software. When using such software, the default parameters, e.g. for gap penalty and extension penalty, are preferably used.

### Exemplary Sequences

TABLE 3
CDR definition according to Kabat

| | | |
|---|---|---|
| Human IgG1 | APELLGGPSVFLFPPKPKDTLMISRTPEVTCVVVDVSHEDPEVKFNWYVDGVEVHNAKTKPREE | 1 |
| CH2—CH3 | QYNSTYRVVSVLTVLHQDWLNGKEYKCKVSNKALPAPIEKTISKAKGQPREPQVYTLPPSRDELT | |
| region | KNQVSLTCLVKGFYPSDIAVEWESNGQPENNYKTTPPVLDSDGSFFLYSKLTVDKSRWQQGNVF | |
| (G1m1) | SCSVMHEALHNHYTQKSLSLSPGK | |
| Human IgG1 | APELLGGPSVFLFPPKPKDTLMISRTPEVTCVVVDVSHEDPEVKFNWYVDGVEVHNAKTKPREE | 2 |
| CH2 CH3 | QYNSTYRVVSVLTVLHQDWLNGKEYKCKVSNKALPAPIEKTISKAKGQPREPQVYTLPPSREEM | |
| region | TKNQVSLTCLVKGFYPSDIAVEWESNGQPENNYKTTPPVLDSDGSFFLYSKLTVDKSRWQQGNV | |
| (G1m3) | FSCSVMHEALHNHYTQKSLSLSPGK | |
| Human IgG2 | APPVAGPSVFLFPPKPKDTLMISRTPEVTCVVVDVSHEDPEVQFNWYVDGVEVHNAKTKPREEQ | 3 |
| CH2 CH3 | FNSTFRVVSVLTVVHQDWLNGKEYKCKVSNKGLPAPIEKTISKTKGQPREPQVYTLPPSREEMTK | |
| region | NQVSLTCLVKGFYPSDISVEWESNGQPENNYKTTPPMLDSDGSFFLYSKLTVDKSRWQQGNVFS | |
| | CSVMHEALHNHYTQKSLSLSPGK | |
| Human IgG3 | APELLGGPSVFLFPPKPKDTLMISRTPEVTCVVVDVSHEDPEVQFKWYVDGVEVHNAKTKPREE | 4 |
| CH2 CH3 | QYNSTFRVVSVLTVLHQDWLNGKEYKCKVSNKALPAPIEKTISKTKGQPREPQVYTLPPSREEMT | |
| region | KNQVSLTCLVKGFYPSDIAVEWESSGQPENNYNTTPPMLDSDGSFFLYSKLTVDKSRWQQGNIFS | |
| | CSVMHEALHNRFTQKSLSLSPGK | |
| Human IgG4 | APEFLGGPSVFLFPPKPKDTLMISRTPEVTCVVVDVSQEDPEVQFNWYVDGVEVHNAKTKPREEQ | 5 |
| CH2 CH3 | FNSTYRVVSVLTVLHQDWLNGKEYKCKVSNKGLPSSIEKTISKAKGQPREPQVYTLPPSQEEMTK | |
| region | NQVSLTCLVKGFYPSDIAVEWESNGQPENNYKTTPPVLDSDGSFFLYSRLTVDKSRWQEGNVFSC | |
| | SVMHEALHNHYTQKSLSLSLGK | |
| Human IgG1 | APELLGGPSVFLFPPKPKDTLMISRTPEVTCVVVDVSHEDPEVKFNWYVDGVEVHNAKTKPREE | 6 |
| CH2 CH3 | QYNSTYRVVSVLTVLHQDWLNGKEYKCKVSNKALGAPIEKTISKAKGQPREPQVYTLPPSRDEL | |
| (G1m1) | TKNQVSLTCLVKGFYPSDIAVEWESNGQPENNYKTTPPVLDSDGSFFLYSKLTVDKSRWQQGNV | |
| region P329G | FSCSVMHEALHNHYTQKSLSLSPGK | |
| Human IgG1 | APEAAGGPSVFLFPPKPKDTLMISRTPEVTCVVVDVSHEDPEVKFNWYVDGVEVHNAKTKPREE | 7 |
| CH2 CH3 | QYNSTYRVVSVLTVLHQDWLNGKEYKCKVSNKALGAPIEKTISKAKGQPREPQVYTLPPSRDEL | |
| (G1m1) | TKNQVSLTCLVKGFYPSDIAVEWESNGQPENNYKTTPPVLDSDGSFFLYSKLTVDKSRWQQGNV | |
| region | FSCSVMHEALHNHYTQKSLSLSPGK | |
| L234A, | | |
| L235A, | | |
| P329G | | |
| Human IgG1 | APELLGGPSVFLFPPKPKDTLMISRTPEVTCVVVDVSHEDPEVKFNWYVDGVEVHNAKTKPREE | 8 |
| CH2 CH3 | QYNSTYRVVSVLTVLHQDWLNGKEYKCKVSNKALGAPIEKTISKAKGQPREPQVYTLPPSREEM | |
| (G1m3) | TKNQVSLTCLVKGFYPSDIAVEWESNGQPENNYKTTPPVLDSDGSFFLYSKLTVDKSRWQQGNV | |
| region P329G | FSCSVMHEALHNHYTQKSLSLSPGK | |
| Human IgG1 | APEAAGGPSVFLFPPKPKDTLMISRTPEVTCVVVDVSHEDPEVKFNWYVDGVEVHNAKTKPREE | 9 |
| CH2 CH3 | QYNSTYRVVSVLTVLHQDWLNGKEYKCKVSNKALGAPIEKTISKAKGQPREPQVYTLPPSREEM | |
| (G1m3) | TKNQVSLTCLVKGFYPSDIAVEWESNGQPENNYKTTPPVLDSDGSFFLYSKLTVDKSRWQQGNV | |
| region | FSCSVMHEALHNHYTQKSLSLSPGK | |
| L234A, | | |
| L235A, | | |
| P329G | | |
| αP329G_VH1 | EVQLVESGGGLVQPGGSLRLSCAASGFDFSRYWMNWVRQAPGKGLEWVGEITPDSSTINYTPSL | 10 |
| | KGRFTISRDNAKNSLYLQMNSLRAEDTAVYYCVRPYDYGAWFASWGQGTLVTVSS | |
| αP329G_VH1, | RYWMN | 11 |
| VH2, VH3 | | |
| HC-CDR1 | | |
| αP329G_VH1 | EITPDSSTINYTPSLKG | 12 |
| HC-CDR2 | | |
| αP329G_VH1, | PYDYGAWFAS | 13 |
| VH2, VH3 | | |
| HC-CDR3 | | |
| αP329G_VH1, | EVQLVESGGGLVQPGGSLRLSCAASGFDFS | 14 |
| VH2 HC-FR1 | | |
| αP329G | WVRQAPGKGLEWVG | 15 |
| VH1, VH2, | | |
| VH3 HC-FR2 | | |
| αP329G_VH1, | RFTISRDNAKNSLYLQMNSLRAEDTAVYYCVR | 16 |
| VH2 HC-FR3 | | |
| αP329G_VH | WGQGTLVTVSS | 17 |
| 1, VH2, VH3 | | |
| HC-FR4 | | |
| αP329G_VH2 | EVQLVESGGGLVQPGGSLRLSCAASGFDFSRYWMNWVRQAPGKGLEWVGEITPDSSTINYAPSL | 18 |
| | KGRFTISRDNAKNSLYLQMNSLRAEDTAVYYCVRPYDYGAWFASWGQGTLVTVSS | |
| αP329G_VH2, | EITPDSSTINYAPSLKG | 19 |
| VH3 HC- | | |
| CDR2 | | |
| αP329G_VH3 | EVQLVESGGGLVQPGGSLRLSCAASGFTFSRYWMNWVRQAPGKGLEWVGEITPDSSTINYAPSL | 20 |
| | KGRFTISRDNAKNSLYLQMNSLRAEDTAVYYCARPYDYGAWFASWGQGTLVTVSS | |
| αP329G_VH3 | EVQLVESGGGLVQPGGSLRLSCAASGFTFS | 21 |
| HC-FR1 | | |
| αP329G_VH3 | RFTISRDNAKNSLYLQMNSLRAEDTAVYYCAR | 22 |
| HC-FR3 | | |
| αP329G_VL1 | QAVVTQEPSLTVSPGGTVTLTCRSSTGAVTTSNYANWVQEKPDHLFTGLIGGTNKRAPGTPARFS | 23 |
| | GSLLGGKAALTLSGAQPEDEAEYYCALWYSNHWVFGGGTKLTVL | |
| αP329G_VL1 | RSSTGAVTTSNYAN | 24 |
| LC-CDR1 | | |
| αP329G_VL1 | GTNKRAP | 25 |
| LC-CDR2 | | |
| αP329G_VL1 | ALWYSNHWV | 26 |
| LC-CDR3 | | |
| αP329G_VL1 | QAVVTQEPSLTVSPGGTVTLTC | 27 |
| LC-FR1 | | |
| αP329G_VL1 | WVQEKPDHLFTGLIG | 28 |
| LC-FR2 | | |
| αP329G_VL1 | GTPARFSGSLLGGKAALTLSGAQPEDEAEYYC | 29 |
| LC-FR3 | | |
| αP329G_VL1 | FGGGTKLTVL | 30 |
| LC-FR4 | | |
| G₄S | GGGGS | 31 |
| (G₄S)3 linker | GGGGSGGGGSGGGGS | 32 |
| αP329G | EVQLVESGGGLVQPGGSLRLSCAASGFDFSRYWMNWVRQAPGKGLEWVGEITPDSSTINYTPSL | 33 |
| VH1VL1 | KGRFTISRDNAKNSLYLQMNSLRAEDTAVYYCVRPYDYGAWFASWGQGTLVTVSSGGGGSGG | |
| scFv | GGSGGGGSQAVVTQEPSLTVSPGGTVTLTCRSSTGAVTTSNYANWVQEKPDHLFTGLIGGTNKR | |
| | APGTPARFSGSLLGGKAALTLSGAQPEDEAEYYCALWYSNHWVFGGGTKLTVL | |
| αP329G | EVQLVESGGGLVQPGGSLRLSCAASGFDFSRYWMNWVRQAPGKGLEWVGEITPDSSTINYAPSL | 34 |
| VH2VL1 | KGRFTISRDNAKNSLYLQMNSLRAEDTAVYYCVRPYDYGAWFASWGQGTLVTVSSGGGGSGG | |
| scFv | GGSGGGGSQAVVTQEPSLTVSPGGTVTLTCRSSTGAVTTSNYANWVQEKPDHLFTGLIGGTNKR | |
| | APGTPARFSGSLLGGKAALTLSGAQPEDEAEYYCALWYSNHWVFGGGTKLTVL | |
| αP329G | EVQLVESGGGLVQPGGSLRLSCAASGFTFSRYWMNWVRQAPGKGLEWVGEITPDSSTINYAPSL | 35 |
| VH3VL1 | KGRFTISRDNAKNSLYLQMNSLRAEDTAVYYCARPYDYGAWFASWGQGTLVTVSSGGGGSGG | |
| scFv | GGSGGGGSQAVVTQEPSLTVSPGGTVTLTCRSSTGAVTTSNYANWVQEKPDHLFTGLIGGTNKR | |
| | APGTPARFSGSLLGGKAALTLSGAQPEDEAEYYCALWYSNHWVFGGGTKLTVL | |
| αPD-1 VH | EVQLLESGGGLVQPGGSLRLSCAASGFSFSSYTMSWVRQAPGKGLEWVATISGGGRDIYYPDSVK | 36 |
| | GRFTISRDNSKNTLYLQMNSLRAEDTAVYYCVLLTGRVYFALDSWGQGTLVTVSS | |
| αPD-1 VL | DIVMTQSPDSLAVSLGERATINCKASESVDTSDNSFIHWYQQKPGQSPKLLIYRSSTLESGVPDRFS | 37 |
| | GSGSGTDFTLTISSLQAEDVAVYYCQQNYDVPWTFGQGTKVEIK | |
| quadruple | APASSSTKKTQLQLEHLLLDLQMILNGINNYKNPKLTRMLTAKFAMPKKATELKHLQCLEEELKP | 38 |
| mutant human | LEEVLNGAQSKNFHLRPRDLISNINVIVLELKGSETTFMCEYADETATIVEFLNRWITFAQSIISTLT | |
| IL2 (IL-2qm | | |
| or IL2v) | | |
| human IL2v | APASSSTKKTQLQLEHLLLDLQMILNGINNYKNPKLTRMLTAKFAMPKKATELKHLQCLEEELKP | 39 |
| Q126T | LEEVLNGAQSKNFHLRPRDLISNINVIVLELKGSETTFMCEYADETATIVEFLNRWITFATSIISTLT | |
| human wt IL- | APTSSSTKKTQLQLEHLLLDLQMILNGINNYKNPKLTRMLTFKFYMPKKATELKHLQCLEEELKP | 40 |
| 2 | LEEVLNLAQSKNFHLRPRDLISNINVIVLELKGSETTFMCEYADETATIVEFLNRWITFAQSIISTLT | |
| Fc P329G | DKTHTCPPCPAPEAAGGPSVFLFPPKPKDTLMISRTPEVTCVVVDVSHEDPEVKFNWYVDGVEVH | 41 |
| LALA knob | NAKTKPREEQYNSTYRVVSVLTVLHQDWLNGKEYKCKVSNKALGAPIEKTISKAKGQPREPQVY | |
| chain | TLPPCRDELTKNQVSLWCLVKGFYPSDIAVEWESNGQPENNYKTTPPVLDSDGSFFLYSKLTVDK | |
| | SRWQQGNVFSCSVMHEALHNHYTQKSLSLSPG | |
| Fc P329G | DKTHTCPPCPAPEAAGGPSVFLFPPKPKDTLMISRTPEVTCVVVDVSHEDPEVKFNWYVDGVEVH | 42 |
| LALA hole | NAKTKPREEQYNSTYRVVSVLTVLHQDWLNGKEYKCKVSNKALGAPIEKTISKAKGQPREPQVC | |
| chain | TLPPSRDELTKNQVSLSCAVKGFYPSDIAVEWESNGQPENNYKTTPPVLDSDGSFFLVSKLTVDKS | |
| | RWQQGNVFSCSVMHEALHNHYTQKSLSLSPGK | |
| Fc P329G | DKTHTCPPCPAPEAAGGPSVFLFPPKPKDTLMISRTPEVTCVVVDVSHEDPEVKFNWYVDGVEVH | 43 |
| LALA knob | NAKTKPREEQYNSTYRVVSVLTVLHQDWLNGKEYKCKVSNKALGAPIEKTISKAKGQPREPQVY | |
| chain (G4S)3 | TLPPCRDELTKNQVSLWCLVKGFYPSDIAVEWESNGQPENNYKTTPPVLDSDGSFFLYSKLTVDK | |
| IL2v | SRWQQGNVFSCSVMHEALHNHYTQKSLSLSPGGGGGSGGGGSGGGGSAPASSSTKKTQLQLEH | |
| | LLLDLQMILNGINNYKNPKLTRMLTAKFAMPKKATELKHLQCLEEELKPLEEVLNGAQSKNFHL | |
| | RPRDLISNINVIVLELKGSETTFMCEYADETATIVEFLNRWITFAQSIISTLT | |
| Fc P329G | DKTHTCPPCPAPEAAGGPSVFLFPPKPKDTLMISRTPEVTCVVVDVSHEDPEVKFNWYVDGVEVH | 44 |
| LALA knob | NAKTKPREEQYNSTYRVVSVLTVLHQDWLNGKEYKCKVSNKALGAPIEKTISKAKGQPREPQVY | |
| chain (G4S)3 | TLPPCRDELTKNQVSLWCLVKGFYPSDIAVEWESNGQPENNYKTTPPVLDSDGSFFLYSKLTVDK | |
| IL2v Q126T | SRWQQGNVFSCSVMHEALHNHYTQKSLSLSPGGGGGSGGGGSGGGGSAPASSSTKKTQLQLEH | |
| | LLLDLQMILNGINNYKNPKLTRMLTAKFAMPKKATELKHLQCLEEELKPLEEVLNGAQSKNFHL | |
| | RPRDLISNINVIVLELKGSETTFMCEYADETATIVEFLNRWITFATSIISTLT | |
| Fc LALA | DKTHTCPPCPAPEAAGGPSVFLFPPKPKDTLMISRTPEVTCVVVDVSHEDPEVKFNWYVDGVEVH | 45 |
| knob chain | NAKTKPREEQYNSTYRVVSVLTVLHQDWLNGKEYKCKVSNKALPAPIEKTISKAKGQPREPQVY | |
| (G4S)3 IL2v | TLPPCRDELTKNQVSLWCLVKGFYPSDIAVEWESNGQPENNYKTTPPVLDSDGSFFLYSKLTVDK | |
| | SRWQQGNVFSCSVMHEALHNHYTQKSLSLSPGGGGGSGGGGSGGGGSAPASSSTKKTQLQLEH | |
| | LLLDLQMILNGINNYKNPKLTRMLTAKFAMPKKATELKHLQCLEEELKPLEEVLNGAQSKNFHL | |
| | RPRDLISNINVIVLELKGSETTFMCEYADETATIVEFLNRWITFAQSIISTLT | |
| Fc WT knob | DKTHTCPPCPAPELLGGPSVFLFPPKPKDTLMISRTPEVTCVVVDVSHEDPEVKFNWYVDGVEVH | 46 |
| chain (G4S)3 | NAKTKPREEQYNSTYRVVSVLTVLHQDWLNGKEYKCKVSNKALPAPIEKTISKAKGQPREPQVY | |
| IL2v Q126T | TLPPCRDELTKNQVSLWCLVKGFYPSDIAVEWESNGQPENNYKTTPPVLDSDGSFFLYSKLTVDK | |
| | SRWQQGNVFSCSVMHEALHNHYTQKSLSLSPGGGGGSGGGGSGGGGSAPASSSTKKTQLQLEH | |
| | LLLDLQMILNGINNYKNPKLTRMLTAKFAMPKKATELKHLQCLEEELKPLEEVLNGAQSKNFHL | |
| | RPRDLISNINVIVLELKGSETTFMCEYADETATIVEFLNRWITFATSIISTLT | |
| IL2v_(G4S)3 | APASSSTKKTQLQLEHLLLDLQMILNGINNYKNPKLTRMLTAKFAMPKKATELKHLQCLEEELKP | 47 |
| Fc P329G | LEEVLNGAQSKNFHLRPRDLISNINVIVLELKGSETTFMCEYADETATIVEFLNRWITFAQSIISTLT | |
| LALAknob | GGGGSGGGGSGGGGSDKTHTCPPCPAPEAAGGPSVFLFPPKPKDTLMISRTPEVTCVVVDVSHED | |
| chain | PEVKFNWYVDGVEVHNAKTKPREEQYNSTYRVVSVLTVLHQDWLNGKEYKCKVSNKALGAPI | |
| | EKTISKAKGQPREPQVYTLPPCRDELTKNQVSLWCLVKGFYPSDIAVEWESNGQPENNYKTTPPV | |
| | LDSDGSFFLYSKLTVDKSRWQQGNVFSCSVMHEALHNHYTQKSLSLSPGK | |
| IL2v Q126T | APASSSTKKTQLQLEHLLLDLQMILNGINNYKNPKLTRMLTAKFAMPKKATELKHLQCLEEELKP | 48 |
| (G4S)3 Fc | LEEVLNGAQSKNFHLRPRDLISNINVIVLELKGSETTFMCEYADETATIVEFLNRWITFATSIISTLT | |
| P329G | GGGGSGGGGSGGGGSDKTHTCPPCPAPEAAGGPSVFLFPPKPKDTLMISRTPEVTCVVVDVSHED | |
| LALA knob | PEVKFNWYVDGVEVHNAKTKPREEQYNSTYRVVSVLTVLHQDWLNGKEYKCKVSNKALGAPI | |
| chain | EKTISKAKGQPREPQVYTLPPCRDELTKNQVSLWCLVKGFYPSDIAVEWESNGQPENNYKTTPPV | |
| | LDSDGSFFLYSKLTVDKSRWQQGNVFSCSVMHEALHNHYTQKSLSLSPGK | |
| IL2v_(G4S)3 | APASSSTKKTQLQLEHLLLDLQMILNGINNYKNPKLTRMLTAKFAMPKKATELKHLQCLEEELKP | 49 |
| Fc LALA | LEEVLNGAQSKNFHLRPRDLISNINVIVLELKGSETTFMCEYADETATIVEFLNRWITFAQSIISTLT | |
| knob chain | GGGGSGGGGSGGGGSDKTHTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWLNGKEYKCKVSNKALPAPIE | |
| | KTISKAKGQPREPQVYTLPPCRDELTKNQVSLWCLVKGFYPSDIAVEWESNGQPENNYKTTPPVL | |
| | DSDGSFFLYSKLTVDKSRWQQGNVFSCSVMHEALHNHYTQKSLSLSPGK | |
| Fc P329G | DKTHTCPPCPAPEAAGGPSVFLFPPKPKDTLMISRTPEVTCVVVDVSHEDPEVKFNWYVDGVEVH | 50 |
| LALA hole | NAKTKPREEQYNSTYRVVSVLTVLHQDWLNGKEYKCKVSNKALGAPIEKTISKAKGQPREPQVC | |
| chain (G4S)3 | TLPPSRDELTKNQVSLSCAVKGFYPSDIAVEWESNGQPENNYKTTPPVLDSDGSFFLVSKLTVDKS | |
| IL2v | RWQQGNVFSCSVMHEALHNHYTQKSLSLSPGKGGGGSGGGGSGGGGSAPASSSTKKTQLQLEH | |
| | LLLDLQMILNGINNYKNPKLTRMLTAKFAMPKKATELKHLQCLEEELKPLEEVLNGAQSKNFHL | |
| | RPRDLISNINVIVLELKGSETTFMCEYADETATIVEFLNRWITFAQSIISTLT | |
| Fc P329G | DKTHTCPPCPAPEAAGGPSVFLFPPKPKDTLMISRTPEVTCVVVDVSHEDPEVKFNWYVDGVEVH | 51 |
| LALA hole | NAKTKPREEQYNSTYRVVSVLTVLHQDWLNGKEYKCKVSNKALGAPIEKTISKAKGQPREPQVC | |
| chain (G4S)3 | TLPPSRDELTKNQVSLSCAVKGFYPSDIAVEWESNGQPENNYKTTPPVLDSDGSFFLVSKLTVDKS | |
| IL2v Q126T | RWQQGNVFSCSVMHEALHNHYTQKSLSLSPGKGGGGSGGGGSGGGGSAPASSSTKKTQLQLEH | |
| | LLLDLQMILNGINNYKNPKLTRMLTAKFAMPKKATELKHLQCLEEELKPLEEVLNGAQSKNFHL | |
| | RPRDLISNINVIVLELKGSETTFMCEYADETATIVEFLNRWITFATSIISTLT | |
| IL2v_(G4S)3 | APASSSTKKTQLQLEHLLLDLQMILNGINNYKNPKLTRMLTAKFAMPKKATELKHLQCLEEELKP | 52 |
| Fc P329G | LEEVLNGAQSKNFHLRPRDLISNINVIVLELKGSETTFMCEYADETATIVEFLNRWITFAQSIISTLT | |
| LALA hole | GGGGSGGGGSGGGGSDKTHTCPPCPAPEAAGGPSVFLFPPKPKDTLMISRTPEVTCVVVDVSHED | |
| chain | PEVKFNWYVDGVEVHNAKTKPREEQYNSTYRVVSVLTVLHQDWLNGKEYKCKVSNKALGAPI | |
| | EKTISKAKGQPREPQVCTLPPSRDELTKNQVSLSCAVKGFYPSDIAVEWESNGQPENNYKTTPPVL | |
| | DSDGSFFLVSKLTVDKSRWQQGNVFSCSVMHEALHNHYTQKSLSLSPGK | |
| IL2v Q126T | APASSSTKKTQLQLEHLLLDLQMILNGINNYKNPKLTRMLTAKFAMPKKATELKHLQCLEEELKP | 53 |
| (G4S)3 Fc | LEEVLNGAQSKNFHLRPRDLISNINVIVLELKGSETTFMCEYADETATIVEFLNRWITFATSIISTLT | |
| P329G | GGGGSGGGGSGGGGSDKTHTCPPCPAPEAAGGPSVFLFPPKPKDTLMISRTPEVTCVVVDVSHED | |
| LALA hole | PEVKFNWYVDGVEVHNAKTKPREEQYNSTYRVVSVLTVLHQDWLNGKEYKCKVSNKALGAPI | |
| chain | EKTISKAKGQPREPQVCTLPPSRDELTKNQVSLSCAVKGFYPSDIAVEWESNGQPENNYKTTPPVL | |
| | DSDGSFFLVSKLTVDKSRWQQGNVFSCSVMHEALHNHYTQKSLSLSPGK | |
| αPD-1 IL2v- | EVQLLESGGGLVQPGGSLRLSCAASGFSFSSYTMSWVRQAPGKGLEWVATISGGGRDIYYPDSVK | 54 |
| HC with IL2v | GRFTISRDNSKNTLYLQMNSLRAEDTAVYYCVLLTGRVYFALDSWGQGTLVTVSSASTKGPSVFP | |
| Fc knob | LAPSSKSTSGGTAALGCLVKDYFPEPVTVSWNSGALTSGVHTFPAVLQSSGLYSLSSVVTVPSSSL | |
| L234A | GTQTYICNVNHKPSNTKVDKKVEPKSCDKTHTCPPCPAPEAAGGPSVFLFPPKPKDTLMISRTPEV | |
| L235A | TCVVVDVSHEDPEVKFNWYVDGVEVHNAKTKPREEQYNSTYRVVSVLTVLHQDWLNGKEYKC | |
| P329G | KVSNKALGAPIEKTISKAKGQPREPQVYTLPPCRDELTKNQVSLWCLVKGFYPSDIAVEWESNGQ | |
| | PENNYKTTPPVLDSDGSFFLYSKLTVDKSRWQQGNVFSCSVMHEALHNHYTQKSLSLSPGGGGG | |
| | SGGGGSGGGGSAPASSSTKKTQLQLEHLLLDLQMILNGINNYKNPKLTRMLTAKFAMPKKATEL | |
| | KHLQCLEEELKPLEEVLNGAQSKNFHLRPRDLISNINVIVLELKGSETTFMCEYADETATIVEFLNR | |
| | WITFAQSIISTLT | |
| αPD-1 | EVQLLESGGGLVQPGGSLRLSCAASGFSFSSYTMSWVRQAPGKGLEWVATISGGGRDIYYPDSVK | 55 |
| HC | GRFTISRDNSKNTLYLQMNSLRAEDTAVYYCVLLTGRVYFALDSWGQGTLVTVSSASTKGPSVFP | |
| Fc hole | LAPSSKSTSGGTAALGCLVKDYFPEPVTVSWNSGALTSGVHTFPAVLQSSGLYSLSSVVTVPSSSL | |
| L234A | GTQTYICNVNHKPSNTKVDKKVEPKSCDKTHTCPPCPAPEAAGGPSVFLFPPKPKDTLMISRTPEV | |
| L235A | TCVVVDVSHEDPEVKFNWYVDGVEVHNAKTKPREEQYNSTYRVVSVLTVLHQDWLNGKEYKC | |
| P329G | KVSNKALGAPIEKTISKAKGQPREPQVCTLPPSRDELTKNQVSLSCAVKGFYPSDIAVEWESNGQP | |
| | ENNYKTTPPVLDSDGSFFLVSKLTVDKSRWQQGNVFSCSVMHEALHNHYTQKSLSLSP | |
| αPD-1 IL2v- | DIVMTQSPDSLAVSLGERATINCKASESVDTSDNSFIHWYQQKPGQSPKLLIYRSSTLESGVPDRFS | 56 |
| LC | GSGSGTDFTLTISSLQAEDVAVYYCQQNYDVPWTFGQGTKVEIKRTVAAPSVFIFPPSDEQLKSGT | |
| | ASVVCLLNNFYPREAKVQWKVDNALQSGNSQESVTEQDSKDSTYSLSSTLTLSKADYEKHKVYA | |
| | CEVTHQGLSSPVTKSFNRGEC | |
| αPD-1 IL2v- | EVQLLESGGGLVQPGGSLRLSCAASGFSFSSYTMSWVRQAPGKGLEWVATISGGGRDIYYPDSV | 57 |
| HC with IL2v | KGRFTISRDNSKNTLYLQMNSLRAEDTAVYYCVLLTGRVYFALDSWGQGTLVTVSSASTKGPSV | |
| Q126T | FPLAPSSKSTSGGTAALGCLVKDYFPEPVTVSWNSGALTSGVHTFPAVLQSSGLYSLSSVVTVPSS | |
| Fc knob | SLGTQTYICNVNHKPSNTKVDKKVEPKSCDKTHTCPPCPAPEAAGGPSVFLFPPKPKDTLMISRTP | |
| L234A | EVTCVVVDVSHEDPEVKFNWYVDGVEVHNAKTKPREEQYNSTYRVVSVLTVLHQDWLNGKEY | |
| L235A | KCKVSNKALGAPIEKTISKAKGQPREPQVYTLPPCRDELTKNQVSLWCLVKGFYPSDIAVEWESN | |
| P329G | GQPENNYKTTPPVLDSDGSFFLYSKLTVDKSRWQQGNVFSCSVMHEALHNHYTQKSLSLSPGGG | |
| | GGSGGGGSGGGGSAPASSSTKKTQLQLEHLLLDLQMILNGINNYKNPKLTRMLTAKFAMPKKAT | |
| | ELKHLQCLEEELKPLEEVLNGAQSKNFHLRPRDLISNINVIVLELKGSETTFMCEYADETATIVEFL | |
| | NRWITFATSIISTLT | |
| Murine | EVQLQESGPGLVKPSQSLSLTCSVTGYSITSSYRWNWIRKFPGNRLEWMGYINSAGISNYNPSLKR | 58 |
| surrogate | RISITRDTSKNQFFLQVNSVTTEDAATYYCARSDNMGTTPFTYWGQGTLVTVSSAKTTPPSVYPL | |
| αPD-1 IL2v- | APGSAAQTNSMVTLGCLVKGYFPEPVTVTWNSGSLSSGVHTFPAVLQSDLYTLSSSVTVPSSTWP | |
| HC with IL2v | SQTVTCNVAHPASSTKVDKKIVPRDCGCKPCICTVPEVSSVFIFPPKPKDVLTITLTPKVTCVVVAIS | |
| | KDDPEVQFSWFVDDVEVHTAQTKPREEQINSTFRSVSELPIMHQDWLNGKEFKCRVNSAAFGAPI | |
| | EKTISKTKGRPKAPQVYTIPPPKEQMAKDKVSLTCMITNFFPEDITVEWQWNGQPAENYDNTQPI | |
| | MDTDGSYFVYSDLNVQKSNWEAGNTFTCSVLHEGLHNHHTEKSLSHSPGGGGGSGGGGSGGGG | |
| | SAPASSSTSSSTAEAQQQQQQQQQQQQHLEQLLMDLQELLSRMENYRNLKLPRMLTAKFALPKQ | |
| | ATELKDLQCLEDELGPLRHVLDGTQSKSFQLEDAENFISNIRVTVVKLKGSDNTFECQFDDESATV | |
| | VDFLRRWIAFAQSIISTSPQ | |
| Murine | EVQLQESGPGLVKPSQSLSLTCSVTGYSITSSYRWNWIRKFPGNRLEWMGYINSAGISNYNPSLKR | 59 |
| surrogate | RISITRDTSKNQFFLQVNSVTTEDAATYYCARSDNMGTTPFTYWGQGTLVTVSSAKTTPPSVYPL | |
| αPD-1 | APGSAAQTNSMVTLGCLVKGYFPEPVTVTWNSGSLSSGVHTFPAVLQSDLYTLSSSVTVPSSTWP | |
| HC | SQTVTCNVAHPASSTKVDKKIVPRDCGCKPCICTVPEVSSVFIFPPKPKDVLTITLTPKVTCVVVAIS | |
| | KDDPEVQFSWFVDDVEVHTAQTKPREEQINSTFRSVSELPIMHQDWLNGKEFKCRVNSAAFGAPI | |
| | EKTISKTKGRPKAPQVYTIPPPKKQMAKDKVSLTCMITNFFPEDITVEWQWNGQPAENYKNTQPI | |
| | MKTDGSYFVYSKLNVQKSNWEAGNTFTCSVLHEGLHNHHTEKSLSHSPGK | |
| Murine | DIVMTQGTLPNPVPSGESVSITCRSSKSLLYSDGKTYLNWYLQRPGQSPQLLIYWMSTRASGVSDR | 60 |
| surrogate | FSGSGSGTDFTLKISGVEAEDVGIYYCQQGLEFPTFGGGTKLELKRTDAAPTVSIFPPSSEQLTSGG | |
| αPD-1 IL2v- | ASVVCFLNNFYPKDINVKWKIDGSERQNGVLNSWTDQDSKDSTYSMSSTLTLTKDEYERHNSYT | |
| LC | CEATHKTSTSPIVKSFNRNEC | |
| CD8a tag | AKPTTTPAPRPPTPAPTIASQPLSLRPEACRPAAGGAVHTRGLDFACDIYIWAPLAGTCGVLLLSL | 61 |
| [ECD]- | VITLYCNHRNRRRVCKCPRPVVKSGDKPSLSARYV | |
| [stalk]- | | |
| [TMD]-[ICD] | | |
| P329G | MGWSCIILFLVATATGVHSEVQLVESGGGLVQPGGSLRLSCAASGFTFSRYWMNWVRQAPGKG | 62 |
| VH3LV1 | LEWVGEITPDSSTINYAPSLKGRFTISRDNAKNSLYLQMNSLRAEDTAVYYCARPYDYGAWFAS | |
| | WGQGTLVTVSSGGGGSGGGGSGGGGSQAVVTQEPSLTVSPGGTVTLTCRSSTGAVTTSNYANW | |
| CD8a tag | VQEKPDHLFTGLIGGTNKRAPGTPARFSGSLLGGKAALTLSGAQPEDEAEYYCALWYSNHWVFG | |
| (with SP) | GGTKLTVLGGGGSAKPTTTPAPRPPTPAPTIASQPLSLRPEACRPAAGGAVHTRGLDFACDIYIWA | |
| [SP]- | PLAGTCGVLLLSLVITLYCNHRNRRRVCKCPRPVVKSGDKPSLSARYV | |
| [VH3VL1 | | |
| scFv]-[CD8a | | |
| ECD]-[stalk]- | | |
| [CD8a TMD]- | | |
| [CD8a ICD] | | |
| P329G | EVQLVESGGGLVQPGGSLRLSCAASGFTFSRYWMNWVRQAPGKGLEWVGEITPDSSTINYAPSL | 63 |
| VH3LV1 | KGRFTISRDNAKNSLYLQMNSLRAEDTAVYYCARPYDYGAWFASWGQGTLVTVSSGGGGSGG | |
| CD8a tag | GGSGGGGSQAVVTQEPSLTVSPGGTVTLTCRSSTGAVTTSNYANWVQEKPDHLFTGLIGGTNKR | |
| (mature) | APGTPARFSGSLLGGKAALTLSGAQPEDEAEYYCALWYSNHWVFGGGTKLTVLGGGGSAKPTT | |
| [VH3VL1 | TPAPRPPTPAPTIASQPLSLRPEACRPAAGGAVHTRGLDFACDIYIWAPLAGTCGVLLLSLVITLYC | |
| scFv]-[CD8a | NHRNRRRVCKCPRPVVKSGDKPSLSARYV | |
| ECD]-[stalk]- | | |
| [CD8a TMD]- | | |
| [CD8a ICD] | | |
| P329G | MGWSCIILFLVATATGVHSEVQLVESGGGLVQPGGSLRLSCAASGFTFSRYWMNWVRQAPGKG | 64 |
| VH3LV1 | LEWVGEITPDSSTINYAPSLKGRFTISRDNAKNSLYLQMNSLRAEDTAVYYCARPYDYGAWFAS | |
| CD8a tag | WGQGTLVTVSSGGGGSGGGGSGGGGSQAVVTQEPSLTVSPGGTVTLTCRSSTGAVTTSNYANW | |
| (with SP, | VQEKPDHLFTGLIGGTNKRAPGTPARFSGSLLGGKAALTLSGAQPEDEAEYYCALWYSNHWVFG | |
| GFP) | GGTKLTVLGGGGSAKPTTTPAPRPPTPAPTIASQPLSLRPEACRPAAGGAVHTRGLDFACDIYIWA | |
| [SP]- | PLAGTCGVLLLSLVITLYCNHRNRRRVCKCPRPVVKSGDKPSLSARYVGSGQCTNYALLKLAGD | |
| [VH3VL1 | VESNPGPVSKGEELFTGVVPILVELDGDVNGHKFSVSGEGEGDATYGKLTLKFICTTGKLPVPWP | |
| scFv]-[CD8a | TLVTTLTYGVQCFSRYPDHMKQHDFFKSAMPEGYVQERTIFFKDDGNYKTRAEVKFEGDTLVNR | |
| ECD]-[stalk]- | IELKGIDFKEDGNILGHKLEYNYNSHNVYIMADKQKNGIKVNFKIRHNIEDGSVQLADHYQQNTP | |
| [CD8a TMD]- | IGDGPVLLPDNHYLSTQSALSKDPNEKRDHMVLLEFVTAAGITLGMDELYK | |
| [CD8a ICD]- | | |
| [E2A]- | | |
| [GFP] | | |
| IL2Ra tag | EMETSQFPGEEKPQASPEGRPESETSCLVTTTDFQIQTEMAATMETSIFTTEYQVAVAGCVFLLISV | 65 |
| [ECD]- | LLLSGLTWQRRQRKSRRTI | |
| [stalk]- | | |
| [TMD]-[ICD] | | |
| P329G | MGWSCIILFLVATATGVHSEVQLVESGGGLVQPGGSLRLSCAASGFTFSRYWMNWVRQAPGKG | 66 |
| VH3LV1 | LEWVGEITPDSSTINYAPSLKGRFTISRDNAKNSLYLQMNSLRAEDTAVYYCARPYDYGAWFAS | |
| IL2Ra tag | WGQGTLVTVSSGGGGSGGGGSGGGGSQAVVTQEPSLTVSPGGTVTLTCRSSTGAVTTSNYANW | |
| (with SP) | VQEKPDHLFTGLIGGTNKRAPGTPARFSGSLLGGKAALTLSGAQPEDEAEYYCALWYSNHWVFG | |
| [SP]-[VH3 | GGTKLTVLGGGGSEMETSQFPGEEKPQASPEGRPESETSCLVTTTDFQIQTEMAATMETSIFTTEY | |
| VL1 scFv]- | QVAVAGCVFLLISVLLLSGLTWQRRQRKSRRTI | |
| [IL2Ra | | |
| stalk]-[IL2Ra | | |
| TMD]- | | |
| [IL2Ra ICD] | | |
| P329G | EVQLVESGGGLVQPGGSLRLSCAASGFTFSRYWMNWVRQAPGKGLEWVGEITPDSSTINYAPSL | 67 |
| VH3LV1 | KGRFTISRDNAKNSLYLQMNSLRAEDTAVYYCARPYDYGAWFASWGQGTLVTVSSGGGGSGG | |
| IL2Ra tag | GGSGGGGSQAVVTQEPSLTVSPGGTVTLTCRSSTGAVTTSNYANWVQEKPDHLFTGLIGGTNKR | |
| (mature) | APGTPARFSGSLLGGKAALTLSGAQPEDEAEYYCALWYSNHWVFGGGTKLTVLGGGGSEMETS | |
| [SP]-[VH3 | QFPGEEKPQASPEGRPESETSCLVTTTDFQIQTEMAATMETSIFTTEYQVAVAGCVFLLISVLLLSG | |
| VL1 scFv]- | LTWQRRQRKSRRTI | |
| [IL2Ra | | |
| stalk]-[IL2ra | | |
| TMD]- | | |
| [IL2Ra ICD] | | |
| P329G | EVQLVESGGGLVQPGGSLRLSCAASGFTFSRYWMNWVRQAPGKGLEWVGEITPDSSTINYAPSL | 68 |
| VH3LV1 | KGRFTISRDNAKNSLYLQMNSLRAEDTAVYYCARPYDYGAWFASWGQGTLVTVSSGGGGSGG | |
| IL2Ra tag | GGSGGGGSQAVVTQEPSLTVSPGGTVTLTCRSSTGAVTTSNYANWVQEKPDHLFTGLIGGTNKR | |
| (with SP, | APGTPARFSGSLLGGKAALTLSGAQPEDEAEYYCALWYSNHWVFGGGTKLTVLGGGGSEMETS | |
| GFP) | QFPGEEKPQASPEGRPESETSCLVTTTDFQIQTEMAATMETSIFTTEYQVAVAGCVFLLISVLLLSG | |
| [SP]-[VH3 | LTWQRRQRKSRRTIGSGQCTNYALLKLAGDVESNPGPVSKGEELFTGVVPILVELDGDVNGHKFS | |
| VL1 scFv]- | VSGEGEGDATYGKLTLKFICTTGKLPVPWPTLVTTLTYGVQCFSRYPDHMKQHDFFKSAMPEGY | |
| [IL2Ra | VQERTIFFKDDGNYKTRAEVKFEGDTLVNRIELKGIDFKEDGNILGHKLEYNYNSHNVYIMADKQ | |
| stalk]-[IL2Ra | KNGIKVNFKIRHNIEDGSVQLADHYQQNTPIGDGPVLLPDNHYLSTQSALSKDPNEKRDHMVLLE | |
| TMD]- | FVTAAGITLGMDELYK | |
| [IL2Ra ICD]- | | |
| [E2A]- | | |
| [GFP] | | |
| IL15Ra tag | QLMPSKSPSTGTTEISSHESSHGTPSQTTAKNWELTASASHQPPGVYPQGHSDTTVAISTSTVLLC | 69 |
| [ECD]- | GLSAVSLLACYLKSRQTPPLASVEMEAMEALPVTWGTSSRDEDLENCSHHL | |
| [stalk]- | | |
| [TMD]-[ICD] | | |
| P329G | MGWSCIILFLVATATGVHSEVQLVESGGGLVQPGGSLRLSCAASGFTFSRYWMNWVRQAPGKG | 70 |
| VH3LV1 | LEWVGEITPDSSTINYAPSLKGRFTISRDNAKNSLYLQMNSLRAEDTAVYYCARPYDYGAWFAS | |
| IL15Ra tag | WGQGTLVTVSSGGGGSGGGGSGGGGSQAVVTQEPSLTVSPGGTVTLTCRSSTGAVTTSNYANW | |
| (with SP) | VQEKPDHLFTGLIGGTNKRAPGTPARFSGSLLGGKAALTLSGAQPEDEAEYYCALWYSNHWVFG | |
| [SP]-[VH3 | GGTKLTVLGGGGSQLMPSKSPSTGTTEISSHESSHGTPSQTTAKNWELTASASHQPPGVYPQGHS | |
| VL1 scFv]- | DTTVAISTSTVLLCGLSAVSLLACYLKSRQTPPLASVEMEAMEALPVTWGTSSRDEDLENCSHHL | |
| [IL15Ra | | |
| stalk]- | | |
| [IL15Ra | | |
| TMD]- | | |
| [IL15Ra | | |
| ICD] | | |
| P329G | EVQLVESGGGLVQPGGSLRLSCAASGFTFSRYWMNWVRQAPGKGLEWVGEITPDSSTINYAPSL | 71 |
| VH3LV1 | KGRFTISRDNAKNSLYLQMNSLRAEDTAVYYCARPYDYGAWFASWGQGTLVTVSSGGGGSGG | |
| IL15Ra tag | GGSGGGGSQAVVTQEPSLTVSPGGTVTLTCRSSTGAVTTSNYANWVQEKPDHLFTGLIGGTNKR | |
| (mature) | APGTPARFSGSLLGGKAALTLSGAQPEDEAEYYCALWYSNHWVFGGGTKLTVLGGGGSQLMPS | |
| [SP]-[VH3 | KSPSTGTTEISSHESSHGTPSQTTAKNWELTASASHQPPGVYPQGHSDTTVAISTSTVLLCGLSAVS | |
| VL1 scFv]- | LLACYLKSRQTPPLASVEMEAMEALPVTWGTSSRDEDLENCSHHL | |
| [IL15Ra | | |
| stalk]- | | |
| [IL15Ra | | |
| TMD]- | | |
| [IL15Ra | | |
| ICD] | | |
| P329G | EVQLVESGGGLVQPGGSLRLSCAASGFTFSRYWMNWVRQAPGKGLEWVGEITPDSSTINYAPSL | 72 |
| VH3LV1 | KGRFTISRDNAKNSLYLQMNSLRAEDTAVYYCARPYDYGAWFASWGQGTLVTVSSGGGGSGG | |
| IL15Ra tag | GGSGGGGSQAVVTQEPSLTVSPGGTVTLTCRSSTGAVTTSNYANWVQEKPDHLFTGLIGGTNKR | |
| (with SP, | APGTPARFSGSLLGGKAALTLSGAQPEDEAEYYCALWYSNHWVFGGGTKLTVLGGGGSQLMPS | |
| GFP) | KSPSTGTTEISSHESSHGTPSQTTAKNWELTASASHQPPGVYPQGHSDTTVAISTSTVLLCGLSAVS | |
| [SP]-[VH3 | LLACYLKSRQTPPLASVEMEAMEALPVTWGTSSRDEDLENCSHHLGSGQCTNYALLKLAGDVES | |
| VL1 scFv]- | NPGPVSKGEELFTGVVPILVELDGDVNGHKFSVSGEGEGDATYGKLTLKFICTTGKLPVPWPTLV | |
| [IL15Ra | TTLTYGVQCFSRYPDHMKQHDFFKSAMPEGYVQERTIFFKDDGNYKTRAEVKFEGDTLVNRIEL | |
| stalk]- | KGIDFKEDGNILGHKLEYNYNSHNVYIMADKQKNGIKVNFKIRHNIEDGSVQLADHYQQNTPIGD | |
| [IL15Ra | GPVLLPDNHYLSTQSALSKDPNEKRDHMVLLEFVTAAGITLGMDELYK | |
| TMD]- | | |
| [IL15Ra | | |
| ICD]-[E2A]- | | |
| [GFP] | | |
| CD28 TD | FWVLVVVGGVLACYSLLVTVAFIIFWV | 73 |
| CD28 CSD | RSKRSRLLHSDYMNMTPRRPGPTRKHYQPYAPPRDFAAYRS | 74 |
| CD3z SSD | RVKFSRSADAPAYQQGQNQLYNELNLGRREEYDVLDKRRGRDPEMGGKPRRKNPQEGLYNELQ | 75 |
| | KDKMAEAYSEIGMKGERRRGKGHDGLYQGLSTATKDTYDALHMQALPPR | |
| CD137 CSD | KRGRKKLLYIFKQPFMRPVQTTQEEDGCSCRFPEEEEGGCEL | 76 |
| (G4S)4 linker | GGGGSGGGGSGGGGSGGGGS | 77 |
| G4S linker | GGGGS | 78 |
| T2Alinker | GEGRGSLLTCGDVEENPGP | 79 |
| CD8stalk | KPTTTPAPRPPTPAPTIASQPLSLRPEACRPAAGGAVHTRGLDFACD | 80 |
| Human CD27 | ATGGCGCGCCCGCATCCGTGGTGGCTGTGCGTGCTGGGCACCCTGGTGGGCCTGAGCGCGAC | 81 |
| | CCCGGCGCCGAAAAGCTGCCCGGAACGCCATTATTGGGCGCAGGGCAAACTGTGCTGCCAGA | |
| | TGTGCGAACCGGGCACCTTTCTGGTGAAAGATTGCGATCAGCATCGCAAAGCGGCGCAGTGC | |
| | GATCCGTGCATTCCGGGCGTGAGCTTTAGCCCGGATCATCATACCCGCCCGCATTGCGAAAGC | |
| | TGCCGCCATTGCAACAGCGGCCTGCTGGTGCGCAACTGCACCATTACCGCGAACGCGGAATG | |
| | CGCGTGCCGCAACGGCTGGCAGTGCCGCGATAAAGAATGCACCGAATGCGATCCGCTGCCGA | |
| | ACCCGAGCCTGACCGCGCGCAGCAGCCAGGCGCTGAGCCCGCATCCGCAGCCGACCCATCTG | |
| | CCGTATGTGAGCGAAATGCTGGAAGCGCGCACCGCGGGCCATATGCAGACCCTGGCGGATTT | |
| | TCGCCAGCTGCCGGCGCGCACCCTGAGCACCCATTGGCCGCCGCAGCGCAGCCTGTGCAGCA | |
| | GCGATTTTATTCGCATTCTGGTGATTTTTAGCGGCATGTTTCTGGTGTTTACCCTGGCGGGCGC | |
| | GCTGTTTCTGCATCAGCGCCGCAAATATCGCAGCAACAAAGGCGAAAGCCCGGTGGAACCGG | |
| | CGGAACCGTGCCATTATAGCTGCCCGCGCGAAGAAGAAGGCAGCACCATTCCGATTCAGGAA | |
| | GATTATCGCAAACCGGAACCGGCGTGCAGCCCG | |
| Human CD27 | MARPHPWWLCVLGTLVGLSATPAPKSCPERHYWAQGKLCCQMCEPGTFLVKDCDQHRKAAQ | 82 |
| | CDPCIPGVSFSPDHHTRPHCESCRHCNSGLLVRNCTITANAECACRNGWQCRDKECTECDPLPNP | |
| | SLTARSSQALSPHPQPTHLPYVSEMLEARTAGHMQTLADFRQLPARTLSTHWPPQRSLCSSDFIRI | |
| | LVIFSGMFLVFTLAGALFLHQRRKYRSNKGESPVEPAEPCHYSCPREEEGSTIPIQEDYRKPEPAC | |
| | SP | |
| Murine CD27 | ATGGCGTGGCCGCCGCCGTATTGGCTGTGCATGCTGGGCACCCTGGTGGGCCTGAGCGCGA | 83 |
| | CCCTGGCGCCGAACAGCTGCCCGGATAAACATTATTGGACCGGCGGCGGCCTGTGCTGCCG | |
| | CATGTGCGAACCGGGCACCTTTTTTGTGAAAGATTGCGAACAGGATCGCACCGCGGCGCAG | |
| | TGCGATCCGTGCATTCCGGGCACCAGCTTTAGCCCGGATTATCATACCCGCCCGCATTGCGA | |
| | AAGCTGCCGCCATTGCAACAGCGGCTTTCTGATTCGCAACTGCACCGTGACCGCGAACGCG | |
| | GAATGCAGCTGCAGCAAAAACTGGCAGTGCCGCGATCAGGAATGCACCGAATGCGATCCGC | |
| | CGCTGAACCCGGCGCTGACCCGCCAGCCGAGCGAAACCCCGAGCCCGCAGCCGCCGCCGAC | |
| | CCATCTGCCGCATGGCACCGAAAAACCGAGCTGGCCGCTGCATCGCCAGCTGCCGAACAGC | |
| | ACCGTGTATAGCCAGCGCAGCAGCCATCGCCCGCTGTGCAGCAGCGATTGCATTCGCATTTT | |
| | TGTGACCTTTAGCAGCATGTTTCTGATTTTTGTGCTGGGCGCGATTCTGTTTTTTCATCAGCG | |
| | CCGCAACCATGGCCCGAACGAAGATCGCCAGGCGGTGCCGGAAGAACCGTGCCCGTATAGC | |
| | TGCCCGCGCGAAGAAGAAGGCAGCGCGATTCCGATTCAGGAAGATTATCGCAAACCGGAAC | |
| | CGGCGTTTTATCCG | |
| Murine CD27 | MAWPPPYWLCMLGTLVGLSATLAPNSCPDKHYWTGGGLCCRMCEPGTFFVKDCEQDRTAAQC | 84 |
| | DPCIPGTSFSPDYHTRPHCESCRHCNSGFLIRNCTVTANAECSCSKNWQCRDQECTECDPPLNPA | |
| | LTRQPSETPSPQPPPTHLPHGTEKPSWPLHRQLPNSTVYSQRSSHRPLCSSDCIRIFVTFSSMFLIFV | |
| | LGAILFFHQRRNHGPNEDRQAVPEEPCPYSCPREEEGSAIPIQEDYRKPEPAFYP | |
| Human CD28 | ATGCTGCGCCTGCTGCTGGCGCTGAACCTGTTTCCGAGCATTCAGGTGACCGGCAACAAAAT | 85 |
| | TCTGGTGAAACAGAGCCCGATGCTGGTGGCGTATGATAACGCGGTGAACCTGAGCTGCAAA | |
| | TATAGCTATAACCTGTTTAGCCGCGAATTTCGCGCGAGCCTGCATAAAGGCCTGGATAGCGC | |
| | GGTGGAAGTGTGCGTGGTGTATGGCAACTATAGCCAGCAGCTGCAGGTGTATAGCAAAACC | |
| | GGCTTTAACTGCGATGGCAAACTGGGCAACGAAAGCGTGACCTTTTATCTGCAGAACCTGTA | |
| | TGTGAACCAGACCGATATTTATTTTTGCAAAATTGAAGTGATGTATCCGCCGCCGTATCTGG | |
| | ATAACGAAAAAAGCAACGGCACCATTATTCATGTGAAAGGCAAACATCTGTGCCCGAGCCC | |
| | GCTGTTTCCGGGCCCGAGCAAACCGTTTTGGGTGCTGGTGGTGGTGGGCGGCGTGCTGGCGT | |
| | GCTATAGCCTGCTGGTGACCGTGGCGTTTATTATTTTTTGGGTGCGCAGCAAACGCAGCCGC | |
| | CTGCTGCATAGCGATTATATGAACATGACCCCGCGCCGCCCGGGCCCGACCCGCAAACATT | |
| | ATCAGCCGTATGCGCCGCCGCGCGATTTTGCGGCGTATCGCAGC | |
| Human CD28 | MLRLLLALNLFPSIQVTGNKILVKQSPMLVAYDNAVNLSCKYSYNLFSREFRASLHKGLDSAVE | 86 |
| | VCVVYGNYSQQLQVYSKTGFNCDGKLGNESVTFYLQNLYVNQTDIYFCKIEVMYPPPYLDNEK | |
| | SNGTIIHVKGKHLCPSPLFPGPSKPFWVLVVVGGVLACYSLLVTVAFIIFWVRSKRSRLLHSDYM | |
| | NMTPRRPGPTRKHYQPYAPPRDFAAYRS | |
| Murine CD28 | ATGACCCTGCGCCTGCTGTTTCTGGCGCTGAACTTTTTTAGCGTGCAGGTGACCGAAAACAA | 87 |
| | AATTCTGGTGAAACAGAGCCCGCTGCTGGTGGTGGATAGCAACGAAGTGAGCCTGAGCTGC | |
| | CGCTATAGCTATAACCTGCTGGCGAAAGAATTTCGCGCGAGCCTGTATAAAGGCGTGAACA | |
| | GCGATGTGGAAGTGTGCGTGGGCAACGGCAACTTTACCTATCAGCCGCAGTTTCGCAGCAA | |
| | CGCGGAATTTAACTGCGATGGCGATTTTGATAACGAAACCGTGACCTTTCGCCTGTGGAACC | |
| | TGCATGTGAACCATACCGATATTTATTTTTGCAAAATTGAATTTATGTATCCGCCGCCGTATC | |
| | TGGATAACGAACGCAGCAACGGCACCATTATTCATATTAAAGAAAAACATCTGTGCCATAC | |
| | CCAGAGCAGCCCGAAACTGTTTTGGGCGCTGGTGGTGGTGGCGGGCGTGCTGTTTTGCTATG | |
| | GCCTGCTGGTGACCGTGGCGCTGTGCGTGATTTGGACCAACAGCCGCCGCAACCGCCTGCTG | |
| | CAGAGCGATTATATGAACATGACCCCGCGCCGCCCGGGCCTGACCCGCAAACCGTATCAGC | |
| | CGTATGCGCCGGCGCGCGATTTTGCGGCGTATCGCCCG | |
| Murine CD28 | MTLRLLFLALNFFSVQVTENKILVKQSPLLVVDSNEVSLSCRYSYNLLAKEFRASLYKGVNSDVE | 88 |
| | VCVGNGNFTYQPQFRSNAEFNCDGDFDNETVTFRLWNLHVNHTDIYFCKIEFMYPPPYLDNERS | |
| | NGTIIHIKEKHLCHTQSSPKLFWALVVVAGVLFCYGLLVTVALCVIWTNSRRNRLLQSDYMNMT | |
| | PRRPGLTRKPYQPYAPARDFAAYRP | |
| Human | ATGGGAAACAGCTGTTACAACATAGTAGCCACTCTGTTGCTGGTCCTCAACTTTGAGAGGAC | 89 |
| CD137 | AAGATCATTGCAGGATCCTTGTAGTAACTGCCCAGCTGGTACATTCTGTGATAATAACAGGA | |
| | ATCAGATTTGCAGTCCCTGTCCTCCAAATAGTTTCTCCAGCGCAGGTGGACAAAGGACCTGT | |
| | GACATATGCAGGCAGTGTAAAGGTGTTTTCAGGACCAGGAAGGAGTGTTCCTCCACCAGCA | |
| | ATGCAGAGTGTGACTGCACTCCAGGGTTTCACTGCCTGGGGGCAGGATGCAGCATGTGTGA | |
| | ACAGGATTGTAAACAAGGTCAAGAACTGACAAAAAAAGGTTGTAAAGACTGTTGCTTTGGG | |
| | ACATTTAACGATCAGAAACGTGGCATCTGTCGACCCTGGACAAACTGTTCTTTGGATGGAAA | |
| | GTCTGTGCTTGTGAATGGGACGAAGGAGAGGGACGTGGTCTGTGGACCATCTCCAGCCGAC | |
| | CTCTCTCCGGGAGCATCCTCTGTGACCCCGCCTGCCCCTGCGAGAGAGCCAGGACACTCTCC | |
| | GCAGATCATCTCCTTCTTTCTTGCGCTGACGTCGACTGCGTTGCTCTTCCTGCTGTTCTTCCTC | |
| | ACGCTCCGTTTCTCTGTTGTTAAACGGGGCAGAAAGAAACTCCTGTATATATTCAAACAACC | |
| | ATTTATGAGACCAGTACAAACTACTCAAGAGGAAGATGGCTGTAGCTGCCGATTTCCAGAA | |
| | GAAGAAGAAGGAGGATGTGAACTGTGA | |
| Human | MGNSCYNIVATLLLVLNFERTRSLQDPCSNCPAGTFCDNNRNQICSPCPPNSFSSAGGQRTCDICR | 90 |
| CD137 | QCKGVFRTRKECSSTSNAECDCTPGFHCLGAGCSMCEQDCKQGQELTKKGCKDCCFGTFNDQK | |
| | RGICRPWTNCSLDGKSVLVNGTKERDVVCGPSPADLSPGASSVTPPAPAREPGHSPQIISFFLALT | |
| | STALLFLLFFLTLRFSVVKRGRKKLLYIFKQPFMRPVQTTQEEDGCSCRFPEEEEGGCEL | |
| Murine | ATGGGCAACAACTGCTATAACGTGGTGGTGATTGTGCTGCTGCTGGTGGGCTGCGAAAAAG | 91 |
| CD137 | TGGGCGCGGTGCAGAACAGCTGCGATAACTGCCAGCCGGGCACCTTTTGCCGCAAATATAA | |
| | CCCGGTGTGCAAAAGCTGCCCGCCGAGCACCTTTAGCAGCATTGGCGGCCAGCCGAACTGC | |
| | AACATTTGCCGCGTGTGCGCGGGCTATTTTCGCTTTAAAAAATTTTGCAGCAGCACCCATAA | |
| | CGCGGAATGCGAATGCATTGAAGGCTTTCATTGCCTGGGCCCGCAGTGCACCCGCTGCGAA | |
| | AAAGATTGCCGCCCGGGCCAGGAACTGACCAAACAGGGCTGCAAAACCTGCAGCCTGGGCA | |
| | CCTTTAACGATCAGAACGGCACCGGCGTGTGCCGCCCGTGGACCAACTGCAGCCTGGATGG | |
| | CCGCAGCGTGCTGAAAACCGGCACCACCGAAAAAGATGTGGTGTGCGGCCCGCCGGTGGTG | |
| | AGCTTTAGCCCGAGCACCACCATTAGCGTGACCCCGGAAGGCGGCCCGGGCGGCCATAGCC | |
| | TGCAGGTGCTGACCCTGTTTCTGGCGCTGACCAGCGCGCTGCTGCTGGCGCTGATTTTTATTA | |
| | CCCTGCTGTTTAGCGTGCTGAAATGGATTCGCAAAAAATTTCCGCATATTTTTAAACAGCCG | |
| | TTTAAAAAAACCACCGGCGCGGCGCAGGAAGAAGATGCGTGCAGCTGCCGCTGCCCGCAGG | |
| | AAGAAGAAGGCGGCGGCGGCGGCTATGAACTG | |
| Murine | MGNNCYNVVVIVLLLVGCEKVGAVQNSCDNCQPGTFCRKYNPVCKSCPPSTFSSIGGQPNCNIC | 92 |
| CD137 | RVCAGYFRFKKFCSSTHNAECECIEGFHCLGPQCTRCEKDCRPGQELTKQGCKTCSLGTFNDQN | |
| | GTGVCRPWTNCSLDGRSVLKTGTTEKDVVCGPPVVSFSPSTTISVTPEGGPGGHSLQVLTLFLAL | |
| | TSALLLALIFITLLFSVLKWIRKKFPHIFKQPFKKTTGAAQEEDACSCRCPQEEEGGGGGYEL | |
| Human OX40 | ATGTGCGTGGGCGCGCGCCGCCTGGGCCGCGGCCCGTGCGCGGCGCTGCTGCTGCTGGGCC | 93 |
| | TGGGCCTGAGCACCGTGACCGGCCTGCATTGCGTGGGCGATACCTATCCGAGCAACGATCG | |
| | CTGCTGCCATGAATGCCGCCCGGGCAACGGCATGGTGAGCCGCTGCAGCCGCAGCCAGAAC | |
| | ACCGTGTGCCGCCCGTGCGGCCCGGGCTTTTATAACGATGTGGTGAGCAGCAAACCGTGCA | |
| | AACCGTGCACCTGGTGCAACCTGCGCAGCGGCAGCGAACGCAAACAGCTGTGCACCGCGAC | |
| | CCAGGATACCGTGTGCCGCTGCCGCGCGGGCACCCAGCCGCTGGATAGCTATAAACCGGGC | |
| | GTGGATTGCGCGCCGTGCCCGCCGGGCCATTTTAGCCCGGGCGATAACCAGGCGTGCAAAC | |
| | CGTGGACCAACTGCACCCTGGCGGGCAAACATACCCTGCAGCCGGCGAGCAACAGCAGCGA | |
| | TGCGATTTGCGAAGATCGCGATCCGCCGGCGACCCAGCCGCAGGAAACCCAGGGCCCGCCG | |
| | GCGCGCCCGATTACCGTGCAGCCGACCGAAGCGTGGCCGCGCACCAGCCAGGGCCCGAGCA | |
| | CCCGCCCGGTGGAAGTGCCGGGCGGCCGCGCGGTGGCGGCGATTCTGGGCCTGGGCCTGGT | |
| | GCTGGGCCTGCTGGGCCCGCTGGCGATTCTGCTGGCGCTGTATCTGCTGCGCCGCGATCAGC | |
| | GCCTGCCGCCGGATGCGCATAAACCGCCGGGCGGCGGCAGCTTTCGCACCCCGATTCAGGA | |
| | AGAACAGGCGGATGCGCATAGCACCCTGGCGAAAATT | |
| Human OX40 | MCVGARRLGRGPCAALLLLGLGLSTVTGLHCVGDTYPSNDRCCHECRPGNGMVSRCSRSQNTV | 94 |
| | CRPCGPGFYNDVVSSKPCKPCTWCNLRSGSERKQLCTATQDTVCRCRAGTQPLDSYKPGVDCA | |
| | PCPPGHFSPGDNQACKPWTNCTLAGKHTLQPASNSSDAICEDRDPPATQPQETQGPPARPITVQP | |
| | TEAWPRTSQGPSTRPVEVPGGRAVAAILGLGLVLGLLGPLAILLALYLLRRDQRLPPDAHKPPGG | |
| | GSFRTPIQEEQADAHSTLAKI | |
| Murine OX40 | ATGTATGTGTGGGTGCAGCAGCCGACCGCGCTGCTGCTGCTGGCGCTGACCCTGGGCGTGAC | 95 |
| | CGCGCGCCGCCTGAACTGCGTGAAACATACCTATCCGAGCGGCCATAAATGCTGCCGCGAA | |
| | TGCCAGCCGGGCCATGGCATGGTGAGCCGCTGCGATCATACCCGCGATACCCTGTGCCATCC | |
| | GTGCGAAACCGGCTTTTATAACGAAGCGGTGAACTATGATACCTGCAAACAGTGCACCCAG | |
| | TGCAACCATCGCAGCGGCAGCGAACTGAAACAGAACTGCACCCCGACCCAGGATACCGTGT | |
| | GCCGCTGCCGCCCGGGCACCCAGCCGCGCCAGGATAGCGGCTATAAACTGGGCGTGGATTG | |
| | CGTGCCGTGCCCGCCGGGCCATTTTAGCCCGGGCAACAACCAGGCGTGCAAACCGTGGACC | |
| | AACTGCACCCTGAGCGGCAAACAGACCCGCCATCCGGCGAGCGATAGCCTGGATGCGGTGT | |
| | GCGAAGATCGCAGCCTGCTGGCGACCCTGCTGTGGGAAACCCAGCGCCCGACCTTTCGCCC | |
| | GACCACCGTGCAGAGCACCACCGTGTGGCCGCGCACCAGCGAACTGCCGAGCCCGCCGACC | |
| | CTGGTGACCCCGGAAGGCCCGGCGTTTGCGGTGCTGCTGGGCCTGGGCCTGGGCCTGCTGGC | |
| | GCCGCTGACCGTGCTGCTGGCGCTGTATCTGCTGCGCAAAGCGTGGCGCCTGCCGAACACCC | |
| | CGAAACCGTGCTGGGGCAACAGCTTTCGCACCCCGATTCAGGAAGAACATACCGATGCGCA | |
| | TTTTACCCTGGCGAAAATT | |
| Murine OX40 | MYVWVQQPTALLLLALTLGVTARRLNCVKHTYPSGHKCCRECQPGHGMVSRCDHTRDTLCHP | 96 |
| | CETGFYNEAVNYDTCKQCTQCNHRSGSELKQNCTPTQDTVCRCRPGTQPRQDSGYKLGVDCVP | |
| | CPPGHFSPGNNQACKPWTNCTLSGKQTRHPASDSLDAVCEDRSLLATLLWETQRPTFRPTTVQS | |
| | TTVWPRTSELPSPPTLVTPEGPAFAVLLGLGLGLLAPLTVLLALYLLRKAWRLPNTPKPCWGNSF | |
| | RTPIQEEHTDAHFTLAKI | |
| Human ICOS | ATGAAAAGCGGCCTGTGGTATTTTTTTCTGTTTTGCCTGCGCATTAAAGTGCTGACCGGCGA | 97 |
| | AATTAACGGCAGCGCGAACTATGAAATGTTTATTTTTCATAACGGCGGCGTGCAGATTCTGT | |
| | GCAAATATCCGGATATTGTGCAGCAGTTTAAAATGCAGCTGCTGAAAGGCGGCCAGATTCT | |
| | GTGCGATCTGACCAAAACCAAAGGCAGCGGCAACACCGTGAGCATTAAAAGCCTGAAATTT | |
| | TGCCATAGCCAGCTGAGCAACAACAGCGTGAGCTTTTTTCTGTATAACCTGGATCATAGCCA | |
| | TGCGAACTATTATTTTTGCAACCTGAGCATTTTTGATCCGCCGCCGTTTAAAGTGACCCTGAC | |
| | CGGCGGCTATCTGCATATTTATGAAAGCCAGCTGTGCTGCCAGCTGAAATTTTGGCTGCCGA | |
| | TTGGCTGCGCGGCGTTTGTGGTGGTGTGCATTCTGGGCTGCATTCTGATTTGCTGGCTGACCA | |
| | AAAAAAAATATAGCAGCAGCGTGCATGATCCGAACGGCGAATATATGTTTATGCGCGCGGT | |
| | GAACACCGCGAAAAAAAGCCGCCTGACCGATGTGACCCTG | |
| Human ICOS | MKSGLWYFFLFCLRIKVLTGEINGSANYEMFIFHNGGVQILCKYPDIVQQFKMQLLKGGQILCDL | 98 |
| | TKTKGSGNTVSIKSLKFCHSQLSNNSVSFFLYNLDHSHANYYFCNLSIFDPPPFKVTLTGGYLHIY | |
| | ESQLCCQLKFWLPIGCAAFVVVCILGCILICWLTKKKYSSSVHDPNGEYMFMRAVNTAKKSRLT | |
| | DVTL | |
| Murine ICOS | ATGAAACCGTATTTTTGCCGCGTGTTTGTGTTTTGCTTTCTGATTCGCCTGCTGACCGGCGAA | 99 |
| | ATTAACGGCAGCGCGGATCATCGCATGTTTAGCTTTCATAACGGCGGCGTGCAGATTAGCTG | |
| | CAAATATCCGGAAACCGTGCAGCAGCTGAAAATGCGCCTGTTTCGCGAACGCGAAGTGCTG | |
| | TGCGAACTGACCAAAACCAAAGGCAGCGGCAACGCGGTGAGCATTAAAAACCCGATGCTGT | |
| | GCCTGTATCATCTGAGCAACAACAGCGTGAGCTTTTTTCTGAACAACCCGGATAGCAGCCAG | |
| | GGCAGCTATTATTTTTGCAGCCTGAGCATTTTTGATCCGCCGCCGTTTCAGGAACGCAACCT | |
| | GAGCGGCGGCTATCTGCATATTTATGAAAGCCAGCTGTGCTGCCAGCTGAAACTGTGGCTGC | |
| | CGGTGGGCTGCGCGGCGTTTGTGGTGGTGCTGCTGTTTGGCTGCATTCTGATTATTTGGTTTA | |
| | GCAAAAAAAAATATGGCAGCAGCGTGCATGATCCGAACAGCGAATATATGTTTATGGCGGC | |
| | GGTGAACACCAACAAAAAAAGCCGCCTGGCGGGCGTGACCAGC | |
| Murine ICOS | MKPYFCRVFVFCFLIRLLTGEINGSADHRMFSFHNGGVQISCKYPETVQQLKMRLFREREVLCEL | 100 |
| | TKTKGSGNAVSIKNPMLCLYHLSNNSVSFFLNNPDSSQGSYYFCSLSIFDPPPFQERNLSGGYLHI | |
| | YESQLCCQLKLWLPVGCAAFVVVLLFGCILIIWFSKKKYGSSVHDPNSEYMFMAAVNTNKKSRL | |
| | AGVTS | |
| Human | ATGATTCATCTGGGCCATATTCTGTTTCTGCTGCTGCTGCCGGTGGCGGCGGCGCAGACCAC | 101 |
| DAP10 | CCCGGGCGAACGCAGCAGCCTGCCGGCGTTTTATCCGGGCACCAGCGGCAGCTGCAGCGGC | |
| | TGCGGCAGCCTGAGCCTGCCGCTGCTGGCGGGCCTGGTGGCGGCGGATGCGGTGGCGAGCC | |
| | TGCTGATTGTGGGCGCGGTGTTTCTGTGCGCGCGCCCGCGCCGCAGCCCGGCGCAGGAAGA | |
| | TGGCAAAGTGTATATTAACATGCCGGGCCGCGGC | |
| Human | MIHLGHILFLLLLPVAAAQTTPGERSSLPAFYPGTSGSCSGCGSLSLPLLAGLVAADAVASLLIVG | 102 |
| DAP10 | AVFLCARPRRSPAQEDGKVYINMPGRG | |
| Murine | ATGGATCCGCCGGGCTATCTGCTGTTTCTGCTGCTGCTGCCGGTGGCGGCGAGCCAGACCAG | 103 |
| DAP10 | CGCGGGCAGCTGCAGCGGCTGCGGCACCCTGAGCCTGCCGCTGCTGGCGGGCCTGGTGGCG | |
| | GCGGATGCGGTGATGAGCCTGCTGATTGTGGGCGTGGTGTTTGTGTGCATGCGCCCGCATGG | |
| | CCGCCCGGCGCAGGAAGATGGCCGCGTGTATATTAACATGCCGGGCCGCGGC | |
| Murine | MDPPGYLLFLLLLPVAASQTSAGSCSGCGTLSLPLLAGLVAADAVMSLLIVGVVFVCMRPHGRP | 104 |
| DAP10 | AQEDGRVYINMPGRG | |
| Human | ATGGGGGGACTTGAACCCTGCAGCAGGCTCCTGCTCCTGCCTCTCCTGCTGGCTGTAAGTGG | 105 |
| DAP12 | TCTCCGTCCTGTCCAGGCCCAGGCCCAGAGCGATTGCAGTTGCTCTACGGTGAGCCCGGGCG | |
| | TGCTGGCAGGGATCGTGATGGGAGACCTGGTGCTGACAGTGCTCATTGCCCTGGCCGTGTAC | |
| | TTCCTGGGCCGGCTGGTCCCTCGGGGGCGAGGGGCTGCGGAGGCAGCGACCCGGAAACAGC | |
| | GTATCACTGAGACCGAGTCGCCTTATCAGGAGCTCCAGGGTCAGAGGTCGGATGTCTACAG | |
| | CGACCTCAACACACAGAGGCCGTATTACAAATGA | |
| Human | MGGLEPCSRLLLLPLLLAVSGLRPVQAQAQSDCSCSTVSPGVLAGIVMGDLVLTVLIALAVYFL | 106 |
| DAP12 | GRLVPRGRGAAEAATRKQRITETESPYQELQGQRSDVYSDLNTQRPYYK | |
| Murine | ATGGGGGCTCTGGAGCCCTCCTGGTGCCTTCTGTTCCTTCCTGTCCTCCTGACTGTGGGAGGA | 107 |
| DAP12 | TTAAGTCCCGTACAGGCCCAGAGTGACACTTTCCCAAGATGCGACTGTTCTTCCGTGAGCCC | |
| | TGGTGTACTGGCTGGGATTGTTCTGGGTGACTTGGTGTTGACTCTGCTGATTGCCCTGGCTGT | |
| | GTACTCTCTGGGCCGCCTGGTCTCCCGAGGTCAAGGGACAGCGGAAGGGACCCGGAAACAA | |
| | CACATTGCTGAGACTGAGTCGCCTTATCAGGAGCTTCAGGGTCAGAGACCAGAAGTATACA | |
| | GTGACCTCAACACACAGAGGCAATATTACAGATGA | |
| Murine | MGALEPSWCLLFLPVLLTVGGLSPVQAQSDTFPRCDCSSVSPGVLAGIVLGDLVLTLLIALAVYS | 108 |
| DAP12 | LGRLVSRGQGTAEGTRKQHIAETESPYQELQGQRPEVYSDLNTQRQYYR | |
| Human CD3z | MKWKALFTAAILQAQLPITEAQSFGLLDPKLCYLLDGILFIYGVILTALFLRVKFSRSADAPAYQQ | 109 |
| | GQNQLYNELNLGRREEYDVLDKRRGRDPEMGGKPQRRKNPQEGLYNELQKDKMAEAYSEIGM | |
| | KGERRRGKGHDGLYQGLSTATKDTYDALHMQALPPR | |
| Human CD3z | ATGAAGTGGAAGGCGCTTTTCACCGCGGCCATCCTGCAGGCACAGTTGCCGATTACAGAGG | 110 |
| | CACAGAGCTTTGGCCTGCTGGATCCCAAACTCTGCTACCTGCTGGATGGAATCCTCTTCATC | |
| | TATGGTGTCATTCTCACTGCCTTGTTCCTGAGAGTGAAGTTCAGCAGGAGCGCAGAGCCCCC | |
| | CGCGTACCAGCAGGGCCAGAACCAGCTCTATAACGAGCTCAATCTAGGACGAAGAGAGGA | |
| | GTACGATGTTTTGGACAAGAGACGTGGCCGGGACCCTGAGATGGGGGGAAAGCCGAGAAG | |
| | GAAGAACCCTCAGGAAGGCCTGTACAATGAACTGCAGAAAGATAAGATGGCGGAGGCCTA | |
| | CAGTGAGATTGGGATGAAAGGCGAGCGCCGGAGGGGCAAGGGGCACGATGGCCTTTACCA | |
| | GGGTCTCAGTACAGCCACCAAGGACACCTACGACGCCCTTCACATGCAGGCCCTGCCCCCTC | |
| | GCTAA | |
| Murine CD3z | MKWKVSVLACILHVRFPGAEAQSFGLLDPKLCYLLDGILFIYGVIITALYLRAKFSRSAETAANL | 111 |
| | QDPNQLYNELNLGRREEYDVLEKKRARDPEMGGKQQRRRNPQEGVYNALQKDKMAEAYSEIG | |
| | TKGERRRGKGHDGLYQGLSTATKDTYDALHMQTLAPR | |
| Murine CD3z | ATGAAGTGGAAAGTGTCTGTTCTCGCCTGCATCCTCCACGTGCGGTTCCCAGGAGCAGAGGC | 112 |
| | ACAGAGCTTTGGTCTGCTGGATCCCAAACTCTGCTACTTGCTAGATGGAATCCTCTTCATCTA | |
| | CGGAGTCATCATCACAGCCCTGTACCTGAGAGCAAAATTCAGCAGGAGTGCAGAGACTGCT | |
| | GCCAACCTGCAGGACCCCAACCAGCTCTACAATGAGCTCAATCTAGGGCGAAGAGAGGAAT | |
| | ATGACGTCTTGGAGAAGAAGCGGGCTCGGGATCCAGAGATGGGAGGCAAACAGCAGAGGA | |
| | GGAGGAACCCCCAGGAAGGCGTATACAATGCACTGCAGAAAGACAAGATGGCAGAAGCCT | |
| | ACAGTGAGATCGGCACAAAAGGCGAGAGGCGGAGAGGCAAGGGGCACGATGGCCTTTACC | |
| | AGGGTCTCAGCACTGCCACCAAGGACACCTATGATGCCCTGCATATGCAGACCCTGGCCCCT | |
| | CGCTAA | |
| Human | MWQLLLPTALLLLVSAGMRTEDLPKAVVFLEPQWYRVLEKDSVTLKCQGAYSPEDNSTQWFH | 113 |
| FCGR3A | NESLISSQASSYFIDAATVDDSGEYRCQTNLSTLSDPVQLEVHIGWLLLQAPRWVFKEEDPIHLRC | |
| | HSWKNTALHKVTYLQNGKGRKYFHHNSDFYIPKATLKDSGSYFCRGLFGSKNVSSETVNITITQ | |
| | GLAVSTISSFFPPGYQVSFCLVMVLLFAVDTGLYFSVKTNIRSSTRDWKDHKFKWRKDPQDK | |
| Human | ATGTGGCAGCTGCTGCTGCCGACCGCGCTGCTGCTGCTGGTGAGCGCGGGCATGCGCACCG | 114 |
| FCGR3A | AAGATCTGCCGAAAGCGGTGGTGTTTCTGGAACCGCAGTGGTATCGCGTGCTGGAAAAAGA | |
| | TAGCGTGACCCTGAAATGCCAGGGCGCGTATAGCCCGGAAGATAACAGCACCCAGTGGTTT | |
| | CATAACGAAAGCCTGATTAGCAGCCAGGCGAGCAGCTATTTTATTGATGCGGCGACCGTGG | |
| | ATGATAGCGGCGAATATCGCTGCCAGACCAACCTGAGCACCCTGAGCGATCCGGTGCAGCT | |
| | GGAAGTGCATATTGGCTGGCTGCTGCTGCAGGCGCCGCGCTGGGTGTTTAAAGAAGAAGAT | |
| | CCGATTCATCTGCGCTGCCATAGCTGGAAAAACACCGCGCTGCATAAAGTGACCTATCTGCA | |
| | GAACGGCAAAGGCCGCAAATATTTTCATCATAACAGCGATTTTTATATTCCGAAAGCGACCC | |
| | TGAAAGATAGCGGCAGCTATTTTTGCCGCGGCCTGTTTGGCAGCAAAAACGTGAGCAGCGA | |
| | AACCGTGAACATTACCATTACCCAGGGCCTGGCGGTGAGCACCATTAGCAGCTTTTTTCCGC | |
| | CGGGCTATCAGGTGAGCTTTTGCCTGGTGATGGTGCTGCTGTTTGCGGTGGATACCGGCCTG | |
| | TATTTTAGCGTGAAAACCAACATTCGCAGCAGCACCCGCGATTGGAAAGATCATAAATTTA | |
| | AATGGCGCAAAGATCCGCAGGATAAA | |
| Murine | MFQNAHSGSQWLLPPLTILLLFAFADRQSAALPKAVVKLDPPWIQVLKEDMVTLMCEGTHNPG | 115 |
| FCGR3A | NSSTQWFHNGRSIRSQVQASYTFKATVNDSGEYRCQMEQTRLSDPVDLGVISDWLLLQTPQRVF | |
| | LEGETITLRCHSWRNKLLNRISFFHNEKSVRYHHYKSNFSIPKANHSHSGDYYCKGSLGSTQHQS | |
| | KPVTITVQDPATTSSISLVWYHTAFSLVMCLLFAVDTGLYFYVRRNLQTPREYWRKSLSIRKHQ | |
| | APQDK | |
| Murine | ATGTTTCAGAATGCACACTCTGGAAGCCAATGGCTACTTCCACCACTGACAATTCTGCTGCT | 116 |
| FCGR3A | GTTTGCTTTTGCAGACAGGCAGAGTGCAGCTCTTCCGAAGGCTGTGGTGAAACTGGACCCCC | |
| | CATGGATCCAGGTGCTCAAGGAAGACATGGTGACACTGATGTGCGAAGGGACCCACAACCC | |
| | TGGGAACTCTTCTACCCAGTGGTTCCACAACGGGAGGTCCATCCGGAGCCAGGTCCAAGCC | |
| | AGTTACACGTTTAAGGCCACAGTCAATGACAGTGGAGAATATCGGTGTCAAATGGAGCAGA | |
| | CCCGCCTCAGCGACCCTGTAGATCTGGGAGTGATTTCTGACTGGCTGCTGCTCCAGACCCCT | |
| | CAGCGGGTGTTTCTGGAAGGGGAAACCATCACGCTAAGGTGCCATAGCTGGAGGAACAAAC | |
| | TACTGAACAGGATCTCATTCTTCCATAATGAAAAATCCGTGAGGTATCATCACTACAAAAGT | |
| | AATTTCTCTATCCCAAAAGCCAACCACAGTCACAGTGGGGACTACTACTGCAAAGGAAGTC | |
| | TAGGAAGTACACAGCACCAGTCCAAGCCTGTCACCATCACTGTCCAAGATCCAGCAACTAC | |
| | ATCCTCCATCTCTCTAGTCTGGTACCACACTGCTTTCTCCCTAGTGATGTGCCTCCTGTTTGC | |
| | AGTGGACACGGGCCTTTATTTCTACGTACGGAGAAATCTTCAAACCCCGAGGGAGTACTGG | |
| | AGGAAGTCCCTGTCAATCAGAAAGCACCAGGCTCCTCAAGACAAGTGA | |
| Human | MGWIRGRRSRHSWEMSEFHNYNLDLKKSDFSTRWQKQRCPVVKSKCRENASPFFFCCFIAVAM | 117 |
| NKG2D | GIRFIIMVAIWSAVFLNSLFNQEVQIPLTESYCGPCPKNWICYKNNCYQFFDESKNWYESQASCM | |
| | SQNASLLKVYSKEDQDLLKLVKSYHWMGLVHIPTNGSWQWEDGSILSPNLLTIIEMQKGDCAL | |
| | YASSFKGYIENCSTPNTYICMQRTV | |
| Human | ATGGGCTGGATTCGCGGCCGCCGCAGCCGCCATAGCTGGGAAATGAGCGAATTTCATAACT | 118 |
| NKG2D | ATAACCTGGATCTGAAAAAAAGCGATTTTAGCACCCGCTGGCAGAAACAGCGCTGCCCGGT | |
| | GGTGAAAAGCAAATGCCGCGAAAACGCGAGCCCGTTTTTTTTTTGCTGCTTTATTGCGGTGG | |
| | CGATGGGCATTCGCTTTATTATTATGGTGGCGATTTGGAGCGCGGTGTTTCTGAACAGCCTG | |
| | TTTAACCAGGAAGTGCAGATTCCGCTGACCGAAAGCTATTGCGGCCCGTGCCCGAAAAACT | |
| | GGATTTGCTATAAAAACAACTGCTATCAGTTTTTTGATGAAAGCAAAAACTGGTATGAAAGC | |
| | CAGGCGAGCTGCATGAGCCAGAACGCGAGCCTGCTGAAAGTGTATAGCAAAGAAGATCAG | |
| | GATCTGCTGAAACTGGTGAAAAGCTATCATTGGATGGGCCTGGTGCATATTCCGACCAACG | |
| | GCAGCTGGCAGTGGGAAGATGGCAGCATTCTGAGCCCGAACCTGCTGACCATTATTGAAAT | |
| | GCAGAAAGGCGATTGCGCGCTGTATGCGAGCAGCTTTAAAGGCTATATTGAAAACTGCAGC | |
| | ACCCCGAACACCTATATTTGCATGCAGCGCACCGTG | |
| Murine | MALIRDRKSHHSEMSKCHNYDLKPAKWDTSQEQQKQRLALTTSQPGENGIIRGRYPIEKLKISP | 119 |
| NKG2D | MFVVRVLAIALAIRFTLNTLMWLAIFKETFQPVLCNKEVPVSSREGYCGPCPNNWICHRNNCYQ | |
| | FFNEEKTWNQSQASCLSQNSSLLKIYSKEEQDFLKLVKSYHWMGLVQIPANGSWQWEDGSSLS | |
| | YNQLTLVEIPKGSCAVYGSSFKAYTEDCANLNTYICMKRAV | |
| Murine | ATGGCGCTGATTCGCGATCGCAAAAGCCATCATAGCGAAATGAGCAAATGCCATAACTATG | 120 |
| NKG2D | ATCTGAAACCGGCGAAATGGGATACCAGCCAGGAACAGCAGAAACAGCGCCTGGCGCTGA | |
| | CCACCAGCCAGCCGGGCGAAAACGGCATTATTCGCGGCCGCTATCCGATTGAAAAACTGAA | |
| | AATTAGCCCGATGTTTGTGGTGCGCGTGCTGGCGATTGCGCTGGCGATTCGCTTTACCCTGA | |
| | ACACCCTGATGTGGCTGGCGATTTTTAAAGAAACCTTTCAGCCGGTGCTGTGCAACAAAGAA | |
| | GTGCCGGTGAGCAGCCGCGAAGGCTATTGCGGCCCGTGCCCGAACAACTGGATTTGCCATC | |
| | GCAACAACTGCTATCAGTTTTTTAACGAAGAAAAAACCTGGAACCAGAGCCAGGCGAGCTG | |
| | CCTGAGCCAGAACAGCAGCCTGCTGAAAATTTATAGCAAAGAAGAACAGGATTTTCTGAAA | |
| | CTGGTGAAAAGCTATCATTGGATGGGCCTGGTGCAGATTCCGGCGAACGGCAGCTGGCAGT | |
| | GGGAAGATGGCAGCAGCCTGAGCTATAACCAGCTGACCCTGGTGGAAATTCCGAAAGGCAG | |
| | CTGCGCGGTGTATGGCAGCAGCTTTAAAGCGTATACCGAAGATTGCGCGAACCTGAACACC | |
| | TATATTTGCATGAAACGCGCGGTG | |
| CD28 | YMNM | 121 |
| YMNM | | |
| CD28 PYAP | PYAP | 122 |
| CD28 FMNM | FMNM | 123 |
| CD28 AYAA | AYAA | 124 |
| Signal peptide | MGWSCIILFLVATATGVHS | 125 |
| Signal peptide | ATGGGATGGAGCTGTATCATCCTCTTCTTGGTAGCAACAGCTACCGGTGTGCACTCC | 126 |
| DNA | | |
| sequence | | |
| Human CD8 | MRNQAPGRPKGATFPPRRPTGSRAPPLAPELRAKQRPGERVMALPVTALLLPLALLLHAARPSQ | 127 |
| | FRVSPLDRTWNLGETVELKCQVLLSNPTSGCSWLFQPRGAAASPTFLLYLSQNKPKAAEGLDTQ | |
| | RFSGKRLGDTFVLTLSDFRRENEGYYFCSALSNSIMYFSHFVPVFLPAKPTTTPAPRPPTPAPTIAS | |
| | QPLSLRPEACRPAAGGAVHTRGLDFACDIYIWAPLAGTCGVLLLSLVITLYCNHRNRRRVCKCP | |
| | RPVVKSGDKPSLSARYV | |
| Human CD8 | ATGCGCAACCAGGCGCCGGGCCGCCCGAAAGGCGCGACCTTTCCGCCGCGCCGCCCGACCG | 128 |
| | GCAGCCGCGCGCCGCCGCTGGCGCCGGAACTGCGCGCGAAACAGCGCCCGGGCGAACGCGT | |
| | GATGGCGCTGCCGGTGACCGCGCTGCTGCTGCCGCTGGCGCTGCTGCTGCATGCGGCGCGCC | |
| | CGAGCCAGTTTCGCGTGAGCCCGCTGGATCGCACCTGGAACCTGGGCGAAACCGTGGAACT | |
| | GAAATGCCAGGTGCTGCTGAGCAACCCGACCAGCGGCTGCAGCTGGCTGTTTCAGCCGCGC | |
| | GGCGCGGCGGCGAGCCCGACCTTTCTGCTGTATCTGAGCCAGAACAAACCGAAAGCGGCGG | |
| | AAGGCCTGGATACCCAGCGCTTTAGCGGCAAACGCCTGGGCGATACCTTTGTGCTGACCCTG | |
| | AGCGATTTTCGCCGCGAAAACGAAGGCTATTATTTTTGCAGCGCGCTGAGCAACAGCATTAT | |
| | GTATTTTAGCCATTTTGTGCCGGTGTTTCTGCCGGCGAAACCGACCACCACCCCGGCGCCGC | |
| | GCCCGCCGACCCCGGCGCCGACCATTGCGAGCCAGCCGCTGAGCCTGCGCCCGGAAGCGTG | |
| | CCGCCCGGCGGCGGGCGGCGCGGTGCATACCCGCGGCCTGGATTTTGCGTGCGATATTTATA | |
| | TTTGGGCGCCGCTGGCGGGCACCTGCGGCGTGCTGCTGCTGAGCCTGGTGATTACCCTGTAT | |
| | TGCAACCATCGCAACCGCCGCCGCGTGTGCAAATGCCCGCGCCCGGTGGTGAAAAGCGGCG | |
| | ATAAACCGAGCCTGAGCGCGCGCTATGTG | |
| Murine CD8 | MASPLTRFLSLNLLLMGESIILGSGEAKPQAPELRIFPKKMDAELGQKVDLVCEVLGSVSQGCSW | 129 |
| | LFQNSSSKLPQPTFVVYMASSHNKITWDEKLNSSKLFSAVRDTNNKYVLTLNKFSKENEGYYFC | |
| | SVISNSVMYFSSVVPVLQKVNSTTTKPVLRTPSPVHPTGTSQPQRPEDCRPRGSVKGTGLDFACDI | |
| | YIWAPLAGICVAPLLSLIITLICYHRSRKRVCKCPRPLVRQEGKPRPSEKIV | |
| Murine CD8 | ATGGCGAGCCCGCTGACCCGCTTTCTGAGCCTGAACCTGCTGCTGATGGGCGAAAGCATTAT | 130 |
| | TCTGGGCAGCGGCGAAGCGAAACCGCAGGCGCCGGAACTGCGCATTTTTCCGAAAAAAATG | |
| | GATGCGGAACTGGGCCAGAAAGTGGATCTGGTGTGCGAAGTGCTGGGCAGCGTGAGCCAGG | |
| | GCTGCAGCTGGCTGTTTCAGAACAGCAGCAGCAAACTGCCGCAGCCGACCTTTGTGGTGTAT | |
| | ATGGCGAGCAGCCATAACAAAATTACCTGGGATGAAAAACTGAACAGCAGCAAACTGTTTA | |
| | GCGCGGTGCGCGATACCAACAACAAATATGTGCTGACCCTGAACAAATTTAGCAAAGAAAA | |
| | CGAAGGCTATTATTTTTGCAGCGTGATTAGCAACAGCGTGATGTATTTTAGCAGCGTGGTGC | |
| | CGGTGCTGCAGAAAGTGAACAGCACCACCACCAAACCGGTGCTGCGCACCCCGAGCCCGGT | |
| | GCATCCGACCGGCACCAGCCAGCCGCAGCGCCCGGAAGATTGCCGCCCGCGCGGCAGCGTG | |
| | AAAGGCACCGGCCTGGATTTTGCGTGCGATATTTATATTTGGGCGCCGCTGGCGGGCATTTG | |
| | CGTGGCGCCGCTGCTGAGCCTGATTATTACCCTGATTTGCTATCATCGCAGCCGCAAACGCG | |
| | TGTGCAAATGCCCGCGCCCGCTGGTGCGCCAGGAAGGCAAACCGCGCCCGAGCGAAAAAAT | |
| | TGTG | |
| Human CD40 | MVRLPLQCVLWGCLLTAVHPEPPTACREKQYLINSQCCSLCQPGQKLVSDCTEFTETECLPCGES | 131 |
| | EFLDTWNRETHCHQHKYCDPNLGLRVQQKGTSETDTICTCEEGWHCTSEACESCVLHRSCSPGF | |
| | GVKQIATGVSDTICEPCPVGFFSNVSSAFEKCHPWTSCETKDLVVQQAGTNKTDVVCGPQDRLR | |
| | ALVVIPIIFGILFAILLVLVFIKKVAKKPTNKAPHPKQEPQEINFPDDLPGSNTAAPVQETLHGCQPV | |
| | TQEDGKESRISVQERQ | |
| Human CD40 | ATGGTGCGGCTGCCCCTGCAGTGTGTGCTGTGGGGCTGCCTCCTGACCGCCGTGCACCCCGA | 132 |
| | ACCACCCACAGCATGCCGTGAAAAGCAGTATCTGATCAACTCTCAGTGCTGTAGCCTGTGTC | |
| | AACCTGGACAGAAACTTGTGTCCGATTGTACGGAGTTCACCGAAACCGAATGTCTCCCTTGC | |
| | GGAGAGTCCGAGTTCCTGGACACCTGGAATCGCGAAACCCACTGCCATCAGCACAAGTATTG | |
| | TGATCCAAACCTGGGCCTCAGGGTGCAACAGAAGGGAACTAGTGAGACGGACACCATCTGT | |
| | ACCTGTGAAGAGGGGTGGCACTGCACCTCTGAGGCGTGTGAGTCATGCGTGTTGCACAGATC | |
| | CTGTTCTCCGGGCTTCGGTGTCAAGCAAATCGCCACCGGCGTGTCCGACACTATCTGTGAGCC | |
| | ATGTCCAGTGGGCTTCTTTAGCAATGTGTCTAGCGCCTTCGAGAAGTGTCACCCGTGGACCTC | |
| | CTGTGAAACTAAGGACCTGGTCGTTCAACAGGCCGGCACCAATAAAACTGATGTGGTCTGTG | |
| | GCCCTCAGGACCGCCTCCGTGCCCTCGTGGTCATCCCGATCATTTTCGGTATCCTGTTCGCCA | |
| | TTTTGCTGGTACTCGTGTTCATCAAAAAGGTGGCGAAAAAGCCAACCAACAAAGCGCCACAC | |
| | CCCAAACAAGAACCACAAGAGATCAACTTCCCAGATGACCTCCCTGGATCCAACACCGCAGC | |
| | CCCTGTGCAGGAGACCCTCCACGGGTGTCAACCAGTGACTCAGGAAGATGGTAAGGAGTCCC | |
| | GCATTTCCGTGCAGGAAAGGCAG | |
| Murine CD40 | MVSLPRLCALWGCLLTAVHLGQCVTCSDKQYLHDGQCCDLCQPGSRLTSHCTALEKTQCHPCD | 133 |
| | SGEFSAQWNREIRCHQHRHCEPNQGLRVKKEGTAESDTVCTCKEGQHCTSKDCEACAQHTPCIP | |
| | GFGVMEMATETTDTVCHPCPVGFFSNQSSLFEKCYPWTSCEDKNLEVLQKGTSQTNVICGLKSR | |
| | MRALLVIPVVMGILITIFGVFLYIKKVVKKPKDNEILPPAARRQDPQEMEDYPGHNTAAPVQETLH | |
| | GCQPVTQEDGKESRISVQERQVTDSIALRPLV | |
| Murine CD40 | ATGGTGTCCTTGCCCCGGCTGTGCGCATTGTGGGGTTGCCTGTTGACTGCCGTGCACTTGGGT | 134 |
| | CAGTGCGTGACCTGCTCCGATAAGCAGTACCTCCACGACGGCCAATGCTGTGACCTGTGTCA | |
| | GCCCGGTTCCAGGCTGACCAGCCACTGCACCGCCCTGGAGAAGACCCAGTGCCATCCCTGCG | |
| | ACTCCGGCGAGTTCTCCGCTCAGTGGAACCGCGAGATCCGCTGCCATCAGCACCGTCACTGC | |
| | GAGCCTAACCAGGGCCTGAGAGTCAAGAAAGAGGGCACCGCTGAGTCCGATACCGTCTGTA | |
| | CCTGCAAGGAGGGCCAGCATTGCACCTCAAAGGACTGTGAGGCTTGTGCCCAGCACACACCC | |
| | TGCATCCCTGGTTTTGGAGTTATGGAGATGGCCACCGAAACAACCGATACTGTATGTCATCCC | |
| | TGCCCTGTGGGATTTTTCAGCAACCAGTCCTCTCTGTTCGAAAAGTGCTACCCCTGGACCAGC | |
| | TGTGAAGATAAGAACCTGGAGGTTCTGCAGAAGGGTACCTCCCAAACAAACGTGATCTGCGG | |
| | CCTGAAGAGCAGGATGCGTGCTCTTCTGGTGATCCCGGTCGTAATGGGCATCCTGATCACCA | |
| | TCTTTGGGGTGTTCCTGTATATCAAAAAGGTAGTGAAAAAGCCTAAGGACAACGAAATCCTG | |
| | CCACCTGCTGCCCGTAGGCAGGATCCCCAGGAGATGGAGGACTACCCTGGTCATAACACTGC | |
| | TGCACCAGTGCAGGAGACACTTCATGGATGCCAGCCCGTGACACAGGAAGACGGAAAGGAA | |
| | AGCCGCATCTCCGTCCAGGAACGCCAGGTCACAGATTCCATCGCCCTGCGCCCGCTCGTG | |
| Signal | MGWSCIILFLVATATGVHS | 135 |
| Peptide | | |
| Human CD8a | AKPTTTPAPRPPTPAPTIASQPLSLRPEACRPAAGGAVHTRGLDFACD | 136 |
| stalk | | |
| Human CD8a | IYIWAPLAGTCGVLLLSLVIT | 137 |
| TMD | | |
| Human | KRGRKKLLYIFKQPFMRPVQTTQEEDGCSCRFPEEEEGGCEL | 138 |
| CD137 ICD | | |
| Human CD3ζ | RVKFSRSADAPAYQQGQNQLYNELNLGRREEYDVLDKRRGRDPEMGGKPQRRKNPQEGLYNEL | 139 |
| ICD | QKDKMAEAYSEIGMKGERRRGKGHDGLYQGLSTATKDTYDALHMQALPPR | |
| eGFP | VSKGEELFTGVVPILVELDGDVNGHKFSVSGEGEGDATYGKLTLKFICTTGKLPVPWPTLVTTLT | 140 |
| | YGVQCFSRYPDHMKQHDFFKSAMPEGYVQERTIFFKDDGNYKTRAEVKFEGDTLVNRIELKGID | |
| | FKEDGNILGHKLEYNYNSHNVYIMADKQKNGIKVNFKIRHNIEDGSVQLADHYQQNTPIGDGPV | |
| | LLPDNHYLSTQSALSKDPNEKRDHMVLLEFVTAAGITLGMDELYK | |
| T2Alinker | GSGEGRGSLLTCGDVEENPGP | 141 |
| E2Alinker | GSGQCTNYALLKLAGDVESNPGP | 142 |
| P329G-CAR | MGWSCIILFLVATATGVHSEVQLVESGGGLVQPGGSLRLSCAASGFTFSRYWMNWVRQAPGKG | 143 |
| (with SP, | LEWVGEITPDSSTINYAPSLKGRFTISRDNAKNSLYLQMNSLRAEDTAVYYCARPYDYGAWFAS | |
| T2A, eGFP) | WGQGTLVTVSSGGGGSGGGGSGGGGSQAVVTQEPSLTVSPGGTVTLTCRSSTGAVTTSNYANW | |
| [SP]- | VQEKPDHLFTGLIGGTNKRAPGTPARFSGSLLGGKAALTLSGAQPEDEAEYYCALWYSNHWVFG | |
| [VH3VL1 | GGTKLTVLGGGGSAKPTTTPAPRPPTPAPTIASQPLSLRPEACRPAAGGAVHTRGLDFACDIYIWA | |
| scFv]-[CD8a | PLAGTCGVLLLSLVITKRGRKKLLYIFKQPFMRPVQTTQEEDGCSCRFPEEEEGGCELRVKFSRSA | |
| stalk]-[CD8a | DAPAYQQGQNQLYNELNLGRREEYDVLDKRRGRDPEMGGKPQRRKNPQEGLYNELQKDKMAE | |
| TMD]- | AYSEIGMKGERRRGKGHDGLYQGLSTATKDTYDALHMQALPPRGSGEGRGSLLTCGDVEENPG | |
| [CD137 | PVSKGEELFTGVVPILVELDGDVNGHKFSVSGEGEGDATYGKLTLKFICTTGKLPVPWPTLVTTL | |
| ICD]-[CD3ζ | TYGVQCFSRYPDHMKQHDFFKSAMPEGYVQERTIFFKDDGNYKTRAEVKFEGDTLVNRIELKGI | |
| ICD]-[T2A]- | DFKEDGNILGHKLEYNYNSHNVYIMADKQKNGIKVNFKIRHNIEDGSVQLADHYQQNTPIGDGP | |
| [eGFP] | VLLPDNHYLSTQSALSKDPNEKRDHMVLLEFVTAAGITLGMDELYK | |
| P329G-CAR | EVQLVESGGGLVQPGGSLRLSCAASGFTFSRYWMNWVRQAPGKGLEWVGEITPDSSTINYAPSL | 144 |
| (with T2A, | KGRFTISRDNAKNSLYLQMNSLRAEDTAVYYCARPYDYGAWFASWGQGTLVTVSSGGGGSGG | |
| eGFP) | GGSGGGGSQAVVTQEPSLTVSPGGTVTLTCRSSTGAVTTSNYANWVQEKPDHLFTGLIGGTNKR | |
| [VH3VL1 | APGTPARFSGSLLGGKAALTLSGAQPEDEAEYYCALWYSNHWVFGGGTKLTVLGGGGSAKPTT | |
| scFv]-[CD8a | TPAPRPPTPAPTIASQPLSLRPEACRPAAGGAVHTRGLDFACDIYIWAPLAGTCGVLLLSLVITKRG | |
| stalk]-[CD8a | RKKLLYIFKQPFMRPVQTTQEEDGCSCRFPEEEEGGCELRVKFSRSADAPAYQQGQNQLYNELNL | |
| TMD]- | GRREEYDVLDKRRGRDPEMGGKPQRRKNPQEGLYNELQKDKMAEAYSEIGMKGERRRGKGHD | |
| [CD137 | GLYQGLSTATKDTYDALHMQALPPRGSGEGRGSLLTCGDVEENPGPVSKGEELFTGVVPILVELD | |
| ICD]-[CD3ζ | GDVNGHKFSVSGEGEGDATYGKLTLKFICTTGKLPVPWPTLVTTLTYGVQCFSRYPDHMKQHDF | |
| ICD]-[T2A]- | FKSAMPEGYVQERTIFFKDDGNYKTRAEVKFEGDTLVNRIELKGIDFKEDGNILGHKLEYNYNSH | |
| [eGFP] | NVYIMADKQKNGIKVNFKIRHNIEDGSVQLADHYQQNTPIGDGPVLLPDNHYLSTQSALSKDPNE | |
| | KRDHMVLLEFVTAAGITLGMDELYK | |
| P329G-CAR | MGWSCIILFLVATATGVHSEVQLVESGGGLVQPGGSLRLSCAASGFTFSRYWMNWVRQAPGKG | 145 |
| (with SP) | LEWVGEITPDSSTINYAPSLKGRFTISRDNAKNSLYLQMNSLRAEDTAVYYCARPYDYGAWFAS | |
| [SP]- | WGQGTLVTVSSGGGGSGGGGSGGGGSQAVVTQEPSLTVSPGGTVTLTCRSSTGAVTTSNYANW | |
| [VH3VL1 | VQEKPDHLFTGLIGGTNKRAPGTPARFSGSLLGGKAALTLSGAQPEDEAEYYCALWYSNHWVFG | |
| scFv]-[CD8a | GGTKLTVLGGGGSAKPTTTPAPRPPTPAPTIASQPLSLRPEACRPAAGGAVHTRGLDFACDIYIWA | |
| stalk]-[CD8a | PLAGTCGVLLLSLVITKRGRKKLLYIFKQPFMRPVQTTQEEDGCSCRFPEEEEGGCELRVKFSRSA | |
| TMD]- | DAPAYQQGQNQLYNELNLGRREEYDVLDKRRGRDPEMGGKPQRRKNPQEGLYNELQKDKMAE | |
| [CD137 | AYSEIGMKGERRRGKGHDGLYQGLSTATKDTYDALHMQALPPR | |
| ICD]-[CD3ζ | | |
| ICD] | | |
| P329G-CAR | EVQLVESGGGLVQPGGSLRLSCAASGFTFSRYWMNWVRQAPGKGLEWVGEITPDSSTINYAPSL | 146 |
| (mature) | KGRFTISRDNAKNSLYLQMNSLRAEDTAVYYCARPYDYGAWFASWGQGTLVTVSSGGGGSGG | |
| [VH3VL] | GGSGGGGSQAVVTQEPSLTVSPGGTVTLTCRSSTGAVTTSNYANWVQEKPDHLFTGLIGGTNKR | |
| scFv]-[CD8a | APGTPARFSGSLLGGKAALTLSGAQPEDEAEYYCALWYSNHWVFGGGTKLTVLGGGGSAKPTT | |
| stalk]-[CD8a | TPAPRPPTPAPTIASQPLSLRPEACRPAAGGAVHTRGLDFACDIYIWAPLAGTCGVLLLSLVITKRG | |
| TMD]- | RKKLLYIFKQPFMRPVQTTQEEDGCSCRFPEEEEGGCELRVKFSRSADAPAYQQGQNQLYNELNL | |
| [CD137 | GRREEYDVLDKRRGRDPEMGGKPQRRKNPQEGLYNELQKDKMAEAYSEIGMKGERRRGKGHD | |
| ICD]-[CD3ζ | GLYQGLSTATKDTYDALHMQALPPR | |
| ICD] | | |
| P329G-CAR | MGWSCIILFLVATATGVHSEVQLVESGGGLVQPGGSLRLSCAASGFTFSRYWMNWVRQAPGKG | 147 |
| (with E2A, | LEWVGEITPDSSTINYAPSLKGRFTISRDNAKNSLYLQMNSLRAEDTAVYYCARPYDYGAWFAS | |
| eGFP) | WGQGTLVTVSSGGGGSGGGGSGGGGSQAVVTQEPSLTVSPGGTVTLTCRSSTGAVTTSNYANW | |
| [VH3VL1 | VQEKPDHLFTGLIGGTNKRAPGTPARFSGSLLGGKAALTLSGAQPEDEAEYYCALWYSNHWVFG | |
| scFv]-[CD8a | GGTKLTVLGGGGSAKPTTTPAPRPPTPAPTIASQPLSLRPEACRPAAGGAVHTRGLDFACDIYIWA | |
| stalk]-[CD8a | PLAGTCGVLLLSLVITKRGRKKLLYIFKQPFMRPVQTTQEEDGCSCRFPEEEEGGCELRVKFSRSA | |
| TMD]- | DAPAYQQGQNQLYNELNLGRREEYDVLDKRRGRDPEMGGKPQRRKNPQEGLYNELQKDKMAE | |
| [CD137 | AYSEIGMKGERRRGKGHDGLYQGLSTATKDTYDALHMQALPPRGSGQCTNYALLKLAGDVESN | |
| ICD]-[CD3ζ | PGPVSKGEELFTGVVPILVELDGDVNGHKFSVSGEGEGDATYGKLTLKFICTTGKLPVPWPTLVT | |
| ICD]-[E2A]- | TLTYGVQCFSRYPDHMKQHDFFKSAMPEGYVQERTIFFKDDGNYKTRAEVKFEGDTLVNRIELK | |
| [eGFP] | GIDFKEDGNILGHKLEYNYNSHNVYIMADKQKNGIKVNFKIRHNIEDGSVQLADHYQQNTPIGDG | |
| | PVLLPDNHYLSTQSALSKDPNEKRDHMVLLEFVTAAGITLGMDELYK | |
| P329G-CAR | MGWSCIILFLVATATGVHSEVQLVESGGGLVQPGGSLRLSCAASGFTFSRYWMNWVRQAPGKG | 148 |
| (with SP) | LEWVGEITPDSSTINYAPSLKGRFTISRDNAKNSLYLQMNSLRAEDTAVYYCARPYDYGAWFAS | |
| [SP]- | WGQGTLVTVSSGGGGSGGGGSGGGGSQAVVTQEPSLTVSPGGTVTLTCRSSTGAVTTSNYANW | |
| [VH3VL1 | VQEKPDHLFTGLIGGTNKRAPGTPARFSGSLLGGKAALTLSGAQPEDEAEYYCALWYSNHWVFG | |
| scFv]-[CD8a | GGTKLTVLGGGGSAKPTTTPAPRPPTPAPTIASQPLSLRPEACRPAAGGAVHTRGLDFACDFWVL | |
| stalk]-[CD28 | VVVGGVLACYSLLVTVAFIIFWVRSKRSRLLHSDYMNMTPRRPGPTRKHYQPYAPPRDFAAYRS | |
| TMD]-[CD28 | RVKFSRSADAPAYQQGQNQLYNELNLGRREEYDVLDKRRGRDPEMGGKPQRRKNPQEGLYNEL | |
| ICD]-[CD3ζ | QKDKMAEAYSEIGMKGERRRGKGHDGLYQGLSTATKDTYDALHMQALPPR | |
| ICD] | | |
| P329G-CAR | EVQLVESGGGLVQPGGSLRLSCAASGFTFSRYWMNWVRQAPGKGLEWVGEITPDSSTINYAPSL | 149 |
| (mature) | KGRFTISRDNAKNSLYLQMNSLRAEDTAVYYCARPYDYGAWFASWGQGTLVTVSSGGGGSGG | |
| [VH3VL1 | GGSGGGGSQAVVTQEPSLTVSPGGTVTLTCRSSTGAVTTSNYANWVQEKPDHLFTGLIGGTNKR | |
| scFv]-[CD8a | APGTPARFSGSLLGGKAALTLSGAQPEDEAEYYCALWYSNHWVFGGGTKLTVLGGGGSAKPTT | |
| stalk]-[CD28 | TPAPRPPTPAPTIASQPLSLRPEACRPAAGGAVHTRGLDFACDFWVLVVVGGVLACYSLLVTVAF | |
| TMD]-[CD28 | IIFWVRSKRSRLLHSDYMNMTPRRPGPTRKHYQPYAPPRDFAAYRSRVKFSRSADAPAYQQGQN | |
| ICD]-[CD3ζ | QLYNELNLGRREEYDVLDKRRGRDPEMGGKPQRRKNPQEGLYNELQKDKMAEAYSEIGMKGE | |
| ICD] | RRRGKGHDGLYQGLSTATKDTYDALHMQALPPR | |
| P329G-CAR | MGWSCIILFLVATATGVHSEVQLVESGGGLVQPGGSLRLSCAASGFTFSRYWMNWVRQAPGKG | 150 |
| (with SP, | LEWVGEITPDSSTINYAPSLKGRFTISRDNAKNSLYLQMNSLRAEDTAVYYCARPYDYGAWFAS | |
| T2A, GFP) | WGQGTLVTVSSGGGGSGGGGSGGGGSQAVVTQEPSLTVSPGGTVTLTCRSSTGAVTTSNYANW | |
| [SP]- | VQEKPDHLFTGLIGGTNKRAPGTPARFSGSLLGGKAALTLSGAQPEDEAEYYCALWYSNHWVFG | |
| [VH3VL1 | GGTKLTVLGGGGSAKPTTTPAPRPPTPAPTIASQPLSLRPEACRPAAGGAVHTRGLDFACDFWVL | |
| scFv]-[CD8a | VVVGGVLACYSLLVTVAFIIFWVRSKRSRLLHSDYMNMTPRRPGPTRKHYQPYAPPRDFAAYRS | |
| stalk]-[CD28 | RVKFSRSADAPAYQQGQNQLYNELNLGRREEYDVLDKRRGRDPEMGGKPQRRKNPQEGLYNEL | |
| TMD]-[CD28 | QKDKMAEAYSEIGMKGERRRGKGHDGLYQGLSTATKDTYDALHMQALPPRGSGEGRGSLLTCG | |
| SD]-[CD3ζ | DVEENPGPVSKGEELFTGVVPILVELDGDVNGHKFSVSGEGEGDATYGKLTLKFICTTGKLPVPW | |
| SD]-[T2A]- | PTLVTTLTYGVQCFSRYPDHMKQHDFFKSAMPEGYVQERTIFFKDDGNYKTRAEVKFEGDTLVN | |
| [eGFP | RIELKGIDFKEDGNILGHKLEYNYNSHNVYIMADKQKNGIKVNFKIRHNIEDGSVQLADHYQQNT | |
| | PIGDGPVLLPDNHYLSTQSALSKDPNEKRDHMVLLEFVTAAGITLGMDELYK | |
| P329G-CAR | EVQLVESGGGLVQPGGSLRLSCAASGFTFSRYWMNWVRQAPGKGLEWVGEITPDSSTINYAPSL | 151 |
| (mature) | KGRFTISRDNAKNSLYLQMNSLRAEDTAVYYCARPYDYGAWFASWGQGTLVTVSSGGGGSGG | |
| “HuR968B” | GGSGGGGSGGGGSQAVVTQEPSLTVSPGGTVTLTCRSSTGAVTTSNYANWVQEKPDHLFTGLIG | |
| [VH3VL1 | GTNKRAPGTPARFSGSLLGGKAALTLSGAQPEDEAEYYCALWYSNHWVFGGGTKLTVLGGGG | |
| scFv]- | SIYIWAPLAGTCGVLLLSLVITLYCKRGRKKLLYIFKQPFMRPVQTTQEEDGCSCRFPEEEEGGCE | |
| [CD8ATD]- | LRVKFSRSADAPAYQQGQNQLYNELNLGRREEYDVLDKRRGRDPEMGGKPRRKNPQEGLYNE | |
| [CD137CSD]- | LQKDKMAEAYSEIGMKGERRRGKGHDGLYQGLSTATKDTYDALHMQALPPR | |
| [CD3zSSD] | | |
| Nucleotide | ATGGCCCTGCCTGTGACAGCCCTACTACTGCCCCTGGCCCTTCTGCTTCATGCTGCTAGACCT | 152 |
| encoding | GAGGTGCAGCTGGTGGAATCTGGGGGGGGCTTGGTTCAGCCTGGGGGCAGCCTGAGACTGA | |
| HuR968B | GCTGTGCTGCCTCTGGCTTCACCTTCAGCAGATATTGGATGAACTGGGTGAGACAAGCCCCT | |
| | GGCAAGGGCCTGGAGTGGGTGGGGGAGATCACCCCTGACAGCAGCACCATCAACTATGCCC | |
| | CTAGCCTGAAGGGCAGATTCACCATCAGCAGAGACAATGCCAAGAACAGCCTGTACCTGCA | |
| | GATGAACAGCCTGAGAGCTGAGGACACAGCTGTGTACTATTGTGCTAGACCCTATGACTAT | |
| | GGGGCCTGGTTTGCTAGCTGGGGCCAAGGCACCCTAGTAACAGTGTCATCTGGGGGGGGAG | |
| | GATCTGGGGGGGGGGGTTCTGGGGGGGGGGGCTCTGGTGGGGGGGGTTCTCAAGCTGTGGT | |
| | AACACAAGAGCCTAGCCTGACAGTGAGCCCTGGGGGCACAGTGACCCTGACCTGCAGAAGC | |
| | AGCACTGGGGCTGTGACCACAAGCAACTATGCCAACTGGGTGCAAGAGAAGCCTGACCACC | |
| | TGTTCACTGGCCTGATTGGGGGCACCAATAAGAGAGCACCTGGCACTCCTGCTAGATTTTCT | |
| | GGCTCACTGCTGGGGGGCAAGGCTGCCTTGACCCTTTCTGGGGCTCAGCCTGAGGATGAGG | |
| | CTGAGTACTACTGTGCTCTCTGGTACAGCAACCACTGGGTGTTTGGGGGGGGCACCAAGCTG | |
| | ACAGTGCTGGGGGGGGGGGGTAGCATCTACATCTGGGCCCCCCTGGCTGGCACATGTGGGG | |
| | TGCTGCTGCTGAGCCTGGTGATCACCCTGTACTGCAAGAGAGGCAGAAAGAAGCTGCTGTA | |
| | CATCTTCAAGCAGCCCTTCATGAGACCTGTGCAGACCACCCAAGAGGAGGATGGCTGCAGC | |
| | TGCAGATTCCCTGAGGAGGAGGAGGGGGGCTGTGAGCTGAGAGTGAAGTTCAGCAGATCTG | |
| | CTGATGCCCCTGCCTATCAGCAAGGGCAGAATCAGCTGTATAATGAGCTCAACCTGGGCAG | |
| | AAGAGAGGAGTATGATGTGCTGGACAAGAGAAGAGGCAGAGACCCTGAGATGGGGGGCAA | |
| | GCCTAGAAGAAAGAACCCCCAAGAGGGCCTGTACAATGAGCTGCAAAAGGACAAGATGGC | |
| | TGAGGCCTACTCTGAGATTGGCATGAAGGGGGAGAGAAGAAGAGGCAAGGGCCATGATGG | |
| | CCTGTACCAAGGCCTGAGCACAGCCACCAAGGACACTTATGATGCCTTGCACATGCAAGCC | |
| | CTGCCTCCAAGATGA | |
| CD8 TD | IYIWAPLAGTCGVLLLSLVITLYC | 153 |
| P329G-CAR | EVQLVESGGGLVQPGGSLRLSCAASGFTFSRYWMNWVRQAPGKGLEWVGEITPDSSTINYAPSL | 154 |
| (mature) | KGRFTISRDNAKNSLYLQMNSLRAEDTAVYYCARPYDYGAWFASWGQGTLVTVSSGGGGSGG | |
| “HuR9684” | GGSGGGGSGGGGSQAVVTQEPSLTVSPGGTVTLTCRSSTGAVTTSNYANWVQEKPDHLFTGLIG | |
| [VH3VL1 | GTNKRAPGTPARFSGSLLGGKAALTLSGAQPEDEAEYYCALWYSNHWVFGGGTKLTVLESKYG | |
| scFv]- | PPCPPCPFWVLVVVGGVLACYSLLVTVAFIIFWVRSKRSRLLHSDYMNMTPRRPGPTRKHYQPY | |
| [CD8ATD]- | APPRDFAAYRSRVKFSRSADAPAYQQGQNQLYNELNLGRREEYDVLDKRRGRDPEMGGKPRR | |
| [CD28CSD]- | KNPQEGLYNELQKDKMAEAYSEIGMKGERRRGKGHDGLYQGLSTATKDTYDALHMQALPPR | |
| [CD3zSSD] | | |
| Nucleotide | ATGGCCCTGCCTGTGACAGCCCTATTACTGCCCCTGGCCCTTCTGTTACATGCTGCTAGACCT | 155 |
| encoding | GAGGTTCAACTGGTGGAGTCTGGGGGGGGCCTAGTGCAGCCTGGGGGCAGCCTGAGACTGA | |
| HuR968B | GCTGTGCTGCCTCTGGCTTCACCTTCAGCAGATACTGGATGAACTGGGTGAGACAAGCCCCT | |
| | GGCAAGGGCCTGGAGTGGGTGGGGGAGATCACCCCTGACAGCAGCACCATCAACTATGCCC | |
| | CTAGCCTGAAGGGCAGATTCACCATCAGCAGAGACAATGCCAAGAACAGCCTGTACCTGCA | |
| | GATGAACAGCCTGAGAGCTGAGGACACAGCTGTGTATTATTGTGCTAGACCATATGACTAT | |
| | GGGGCCTGGTTTGCCTCTTGGGGCCAAGGCACACTGGTTACAGTGAGTTCTGGGGGGGGGG | |
| | GTTCTGGAGGGGGGGGATCTGGGGGTGGAGGTTCTGGGGGGGGGGGCAGTCAAGCTGTGGT | |
| | GACCCAAGAGCCTAGCCTGACAGTGTCCCCTGGGGGCACAGTCACCCTGACCTGCAGAAGC | |
| | AGCACTGGGGCTGTGACCACAAGCAACTATGCCAACTGGGTGCAAGAGAAGCCTGACCACC | |
| | TGTTCACTGGCCTGATTGGGGGCACCAACAAAAGAGCCCCTGGCACCCCTGCTAGATTCTCT | |
| | GGAAGCCTGTTGGGGGGCAAGGCTGCCCTGACCCTATCTGGGGCACAGCCTGAGGATGAGG | |
| | CTGAGTACTACTGTGCCCTCTGGTACAGCAACCACTGGGTGTTTGGGGGGGGCACCAAACTG | |
| | ACAGTGTTGGAGAGCAAGTATGGCCCCCCCTGTCCTCCCTGTCCCTTTTGGGTGCTGGTGGT | |
| | TGTGGGGGGGGTGCTGGCCTGCTACAGCCTGCTGGTGACAGTGGCCTTCATCATCTTCTGGG | |
| | TGAGAAGCAAGAGAAGCAGACTGCTGCACTCTGACTACATGAACATGACCCCTAGAAGACC | |
| | TGGCCCCACAAGAAAGCACTATCAGCCCTATGCCCCCCCTAGAGACTTTGCTGCCTACAGAA | |
| | GCAGAGTGAAGTTCAGCAGATCTGCTGATGCCCCTGCCTATCAGCAAGGGCAGAATCAGCT | |
| | GTATAATGAGCTCAACCTGGGCAGAAGAGAGGAGTATGATGTGCTGGACAAGAGAAGAGG | |
| | CAGAGACCCTGAGATGGGGGGCAAGCCTAGAAGAAAGAACCCCCAAGAGGGCCTGTACAA | |
| | TGAGCTGCAAAAGGACAAGATGGCTGAGGCCTACTCTGAGATTGGCATGAAGGGGGAGAG | |
| | AAGAAGAGGCAAGGGCCATGATGGCCTGTACCAAGGCCTGAGCACAGCCACCAAGGACAC | |
| | CTATGATGCCCTACATATGCAAGCTCTGCCCCCTAGATGA | |
| IgG4m | ESKYGPPCPPCP | 156 |
| linker | | |
| Human | IQNPDPAVYQLRDSKSSDKSVCLFTDFDSQTNVSQSKDSDVYITDKTVLDMRSMDFKSNSAVAW | 157 |
| TRAC | SNKSDFACANAFNNSIIPEDTFFPSPESSCDVKLVEKSFETDTNLNFQNLSVIGFRILLLKVAGFNLL | |
| (UniProt: | MTLRLWSS | |
| P01848) | | |
| Human | IQNPDPAVYQLRDSKSSDKSVCLFTDFDSQTNVSQSKDSDVYITDKTVLDMRSMDFKSNSAVAW | 158 |
| TRAC ECD | SNKSDFACANAFNNSIIPEDTFFPSPESSCDVKLVEKSFETDTNLNFQNLS | |
| Human | VIGFRILLLKVAGFNLLMTLRLW | 159 |
| TRAC TMD | | |
| Human | SS | 160 |
| TRAC ICD | | |
| Human | DLNKVFPPEVAVFEPSEAEISHTQKATLVCLATGFFPDHVELSWWVNGKEVHSGVSTDPQPLKEQ | 161 |
| TRBC1 | PALNDSRYCLSSRLRVSATFWQNPRNHFRCQVQFYGLSENDEWTQDRAKPVTQIVSAEAWGRA | |
| (UniProt: | DCGFTSVSYQQGVLSATILYEILLGKATLYAVLVSALVLMAMVKRKDF | |
| P01850) | | |
| Human | DLNKVFPPEVAVFEPSEAEISHTQKATLVCLATGFFPDHVELSWWVNGKEVHSGVSTDPQPLKEQ | 162 |
| TRBC1 ECD | PALNDSRYCLSSRLRVSATFWQNPRNHFRCQVQFYGLSENDEWTQDRAKPVTQIVSAEAWGRA | |
| | DCGFTSVSYQQGVLSATILYE | |
| Human | ILLGKATLYAVLVSALVLMAM | 163 |
| TRBC1 TMD | | |
| Human | VKRKDF | 164 |
| TRBC1 ICD | | |
| Human | DLKNVFPPKVAVFEPSEAEISHTQKATLVCLATGFYPDHVELSWWVNGKEVHSGVSTDPQPLKE | 165 |
| TRBC2 | QPALNDSRYCLSSRLRVSATFWQNPRNHFRCQVQFYGLSENDEWTQDRAKPVTQIVSAEAWGR | |
| (UniProt: | ADCGFTSESYQQGVLSATILYEILLGKATLYAVLVSALVLMAMVKRKDSRG | |
| A0A5B9) | | |
| Human | DLKNVFPPKVAVFEPSEAEISHTQKATLVCLATGFYPDHVELSWWVNGKEVHSGVSTDPQPLKE | 166 |
| TRBC2 ECD | QPALNDSRYCLSSRLRVSATFWQNPRNHFRCQVQFYGLSENDEWTQDRAKPVTQIVSAEAWGR | |
| | ADCGFTSESYQQGVLSA | |
| Human | TILYEILLGKATLYAVLVSALVL | 167 |
| TRBC2 TMD | | |
| Human | MAMVKRKDSRG | 168 |
| TRBC2 ICD | | |
| Human | DKQLDADVSPKPTIFLPSIAETKLQKAGTYLCLLEKFFPDVIKIHWQEKKSNTILGSQEGNTMKTN | 169 |
| TCRγ1 | DTYMKFSWLTVPEKSLDKEHRCIVRHENNKNGVDQEIIFPPIKTDVITMDPKDNCSKDANDTLLL | |
| (UniProt: | QLTNTSAYYMYLLLLLKSVVYFAIITCCLLRRTAFCCNGEKS | |
| POCF51) | | |
| Human | DKQLDADVSPKPTIFLPSIAETKLQKAGTYLCLLEKFFPDVIKIHWQEKKSNTILGSQEGNTMKTN | 170 |
| TCRγ1 ECD | DTYMKFSWLTVPEKSLDKEHRCIVRHENNKNGVDQEIIFPPIKTDVITMDPKDNCSKDANDTLLL | |
| | QLTNTSA | |
| Human | YYMYLLLLLKSVVYFAIITCCLL | 171 |
| TCRγ1 TMD | | |
| Human | RRTAFCCNGEKS | 172 |
| TCRγ1 ICD | | |
| Human | DKQLDADVSPKPTIFLPSIAETKLQKAGTYLCLLEKFFPDIIKIHWQEKKSNTILGSQEGNTMKTND | 173 |
| TCRγ2 | TYMKFSWLTVPEESLDKEHRCIVRHENNKNGIDQEIIFPPIKTDVTTVDPKYNYSKDANDVITMDP | |
| (UniProt: | KDNWSKDANDTLLLQLTNTSAYYTYLLLLLKSVVYFAIITCCLLRRTAFCCNGEKS | |
| P03986) | | |
| Human | DKQLDADVSPKPTIFLPSIAETKLQKAGTYLCLLEKFFPDIIKIHWQEKKSNTILGSQEGNTMKTND | 174 |
| TCRγ2 ECD | TYMKFSWLTVPEESLDKEHRCIVRHENNKNGIDQEIIFPPIKTDVTTVDPKYNYSKDANDVITMDP | |
| | KDNWSKDANDTLLLQLTNTSA | |
| Human | YYTYLLLLLKSVVYFAIITCCLL | 175 |
| TCRγ2 TMD | | |
| Human | RRTAFCCNGEKS | 176 |
| TCRγ2 ICD | | |
| Human | SQPHTKPSVFVMKNGTNVACLVKEFYPKDIRINLVSSKKITEFDPAIVISPSGKYNAVKLGKYEDS | 177 |
| TRDC | NSVTCSVQHDNKTVHSTDFEVKTDSTDHVKPKETENTKQPSKSCHKPKAIVHTEKVNMMSLTVL | |
| (UniProt: | GLRMLFAKTVAVNFLLTAKLFFL | |
| B7Z8K6) | | |
| Human | SQPHTKPSVFVMKNGTNVACLVKEFYPKDIRINLVSSKKITEFDPAIVISPSGKYNAVKLGKYEDS | 178 |
| TRDC ECD | NSVTCSVQHDNKTVHSTDFEVKTDSTDHVKPKETENTKQPSKSCHKPKAIVHTEKVNMMSLTV | |
| Human | LGLRMLFAKTVAVNFLLTAKLFF | 179 |
| TRDC TMD | | |
| Human | L | 180 |
| TRDC ICD | | |
| Human CD38 | MQSGTHWRVLGLCLLSVGVWGQDGNEEMGGITQTPYKVSISGTTVILTCPQYPGSEILWQHNDK | 181 |
| (UniProt: | NIGGDEDDKNIGSDEDHLSLKEFSELEQSGYYVCYPRGSKPEDANFYLYLRARVCENCMEMDVM | |
| P07766) | SVATIVIVDICITGGLLLLVYYWSKNRKAKAKPVTRGAGAGGRQRGQNKERPPPVPNPDYEPIRK | |
| | GQRDLYSGLNQRRI | |
| Human CD3ϵ | MQSGTHWRVLGLCLLSVGVWGQ | 182 |
| SP | | |
| Human CD3ϵ | DGNEEMGGITQTPYKVSISGTTVILTCPQYPGSEILWQHNDKNIGGDEDDKNIGSDEDHLSLKEFS | 183 |
| ECD | ELEQSGYYVCYPRGSKPEDANFYLYLRARVCENCMEMD | |
| Human CD3ϵ | VMSVATIVIVDICITGGLLLLVYYWS | 184 |
| TMD | | |
| Human CD3ϵ | KNRKAKAKPVTRGAGAGGRQRGQNKERPPPVPNPDYEPIRKGQRDLYSGLNQRRI | 185 |
| ICD | | |
| Mature | DGNEEMGGITQTPYKVSISGTTVILTCPQYPGSEILWQHNDKNIGGDEDDKNIGSDEDHLSLKEFS | 186 |
| human CD3ϵ | ELEQSGYYVCYPRGSKPEDANFYLYLRARVCENCMEMDVMSVATIVIVDICITGGLLLLVYYWS | |
| | KNRKAKAKPVTRGAGAGGRQRGQNKERPPPVPNPDYEPIRKGQRDLYSGLNQRRI | |
| Human CD3δ | MEHSTFLSGLVLATLLSQVSPFKIPIEELEDRVFVNCNTSITWVEGTVGTLLSDITRLDLGKRILDPR | 187 |
| (UniProt: | GIYRCNGTDIYKDKESTVQVHYRMCQSCVELDPATVAGIIVTDVIATLLLALGVFCFAGHETGRL | |
| P04234) | SGAADTQALLRNDQVYQPLRDRDDAQYSHLGGNWARNK | |
| Human CD3δ | MEHSTFLSGLVLATLLSQVSP | 188 |
| SP | | |
| Human CD3δ | FKIPIEELEDRVFVNCNTSITWVEGTVGTLLSDITRLDLGKRILDPRGIYRCNGTDIYKDKESTVQV | 189 |
| ECD | HYRMCQSCVELDPATVA | |
| Human CD3δ | GIIVTDVIATLLLALGVFCFA | 190 |
| TMD | | |
| Human CD3δ | GHETGRLSGAADTQALLRNDQVYQPLRDRDDAQYSHLGGNWARNK | 191 |
| ICD | | |
| Mature | FKIPIEELEDRVFVNCNTSITWVEGTVGTLLSDITRLDLGKRILDPRGIYRCNGTDIYKDKESTVQV | 192 |
| human CD3δ | HYRMCQSCVELDPATVAGIIVTDVIATLLLALGVFCFAGHETGRLSGAADTQALLRNDQVYQPL | |
| | RDRDDAQYSHLGGNWARNK | |
| Human CD3γ | MEHSTFLSGLVLATLLSQVSPFKIPIEELEDRVFVNCNTSITWVEGTVGTLLSDITRLDLGKRILDPR | 193 |
| (UniProt: | GIYRCNGTDIYKDKESTVQVHYRMCQSCVELDPATVAGIIVTDVIATLLLALGVFCFAGHETGRL | |
| P04234) | SGAADTQALLRNDQVYQPLRDRDDAQYSHLGGNWARNK | |
| Human CD3γ | MEHSTFLSGLVLATLLSQVSP | 194 |
| SP | | |
| Human CD3γ | FKIPIEELEDRVFVNCNTSITWVEGTVGTLLSDITRLDLGKRILDPRGIYRCNGTDIYKDKESTVQV | 195 |
| ECD | HYRMCQSCVELDPATVA | |
| Human CD3γ | GIIVTDVIATLLLALGVFCFA | 196 |
| TMD | | |
| Human CD3γ | GHETGRLSGAADTQALLRNDQVYQPLRDRDDAQYSHLGGNWARNK | 197 |
| ICD | | |
| Mature | FKIPIEELEDRVFVNCNTSITWVEGTVGTLLSDITRLDLGKRILDPRGIYRCNGTDIYKDKESTVQV | 198 |
| human CD3γ | HYRMCQSCVELDPATVAGIIVTDVIATLLLALGVFCFAGHETGRLSGAADTQALLRNDQVYQPL | |
| | RDRDDAQYSHLGGNWARNK | |
| Human CD3ζ | MKWKALFTAAILQAQLPITEAQSFGLLDPKLCYLLDGILFIYGVILTALFLRVKFSRSADAPAYQQ | 199 |
| (UniProt: | GQNQLYNELNLGRREEYDVLDKRRGRDPEMGGKPQRRKNPQEGLYNELQKDKMAEAYSEIGM | |
| P20963-1) | KGERRRGKGHDGLYQGLSTATKDTYDALHMQALPPR | |
| Human | MKWKALFTAAILQAQLPITEA | 200 |
| CDζ CD3η | | |
| SP | | |
| Human | QSFGLLDPK | 201 |
| CD3ζ CD3η | | |
| ECD | | |
| Human | LCYLLDGILFIYGVILTALFL | 202 |
| CD3ζ CD3η | | |
| TMD | | |
| Human CD3ζ | RVKFSRSADAPAYQQGQNQLYNELNLGRREEYDVLDKRRGRDPEMGGKPQRRKNPQEGLYNEL | 203 |
| ICD | QKDKMAEAYSEIGMKGERRRGKGHDGLYQGLSTATKDTYDALHMQALPPR | |
| Mature | QSFGLLDPKLCYLLDGILFIYGVILTALFLRVKFSRSADAPAYQQGQNQLYNELNLGRREEYDVL | 204 |
| human CD3ζ | DKRRGRDPEMGGKPQRRKNPQEGLYNELQKDKMAEAYSEIGMKGERRRGKGHDGLYQGLSTA | |
| | TKDTYDALHMQALPPR | |
| Human CD3η | MKWKALFTAAILQAQLPITEAQSFGLLDPKLCYLLDGILFIYGVILTALFLRVKFSRSADAPAYQQ | 205 |
| (UniProt: | GQNQLYNELNLGRREEYDVLDKRRGRDPEMGGKPRRKNPQEGLYNELQKDKMAEAYSEIGMK | |
| P20963-2) | GERRRGKGHDGLYQGLSTATKDTYDALHMQALPPR | |
| Human CD3η | RVKFSRSADAPAYQQGQNQLYNELNLGRREEYDVLDKRRGRDPEMGGKPRRKNPQEGLYNELQ | 206 |
| ICD | KDKMAEAYSEIGMKGERRRGKGHDGLYQGLSTATKDTYDALHMQALPPR | |
| Mature | QSFGLLDPKLCYLLDGILFIYGVILTALFLRVKFSRSADAPAYQQGQNQLYNELNLGRREEYDVL | 207 |
| human CD3η | DKRRGRDPEMGGKPRRKNPQEGLYNELQKDKMAEAYSEIGMKGERRRGKGHDGLYQGLSTAT | |
| | KDTYDALHMQALPPR | |
| Human | IQNPDPAVYQLRDSKSSDKSVCLFTDFDSQTNVSQSKDSDVYITDKCVLDMRSMDFKSNSAVAW | 208 |
| TRAC_T47C | SNKSDFACANAFNNSIIPEDTFFPSPESSCDVKLVEKSFETDTNLNFQNLSVIGFRILLLKVAGFNLL | |
| | MTLRLWSS | |
| Human | DLNKVFPPEVAVFEPSEAEISHTQKATLVCLATGFFPDHVELSWWVNGKEVHSGVCTDPQPLKE | 209 |
| TRBC1_S56C | QPALNDSRYCLSSRLRVSATFWQNPRNHFRCQVQFYGLSENDEWTQDRAKPVTQIVSAEAWGR | |
| | ADCGFTSVSYQQGVLSATILYEILLGKATLYAVLVSALVLMAMVKRKDF | |
| Human | DLKNVFPPKVAVFEPSEAEISHTQKATLVCLATGFYPDHVELSWWVNGKEVHSGVCTDPQPLKE | 210 |
| TRBC2_S56C | QPALNDSRYCLSSRLRVSATFWQNPRNHFRCQVQFYGLSENDEWTQDRAKPVTQIVSAEAWGR | |
| | ADCGFTSESYQQGVLSATILYEILLGKATLYAVLVSALVLMAMVKRKDSRG | |
| P329G | MGWSCIILFLVATATGVHSEVQLVESGGGLVQPGGSLRLSCAASGFDFSRYWMNWVRQAPGKG | 211 |
| VH1VL1 | LEWVGEITPDSSTINYTPSLKGRFTISRDNAKNSLYLQMNSLRAEDTAVYYCVRPYDYGAWFAS | |
| CD3ϵ(with | WGQGTLVTVSSGGGGSGGGGSGGGGSQAVVTQEPSLTVSPGGTVTLTCRSSTGAVTTSNYANW | |
| SP, T2A, | VQEKPDHLFTGLIGGTNKRAPGTPARFSGSLLGGKAALTLSGAQPEDEAEYYCALWYSNHWVFG | |
| eGFP) | GGTKLTVLGGGGSGGGGSGGGGSDGNEEMGGITQTPYKVSISGTTVILTCPQYPGSEILWQHNDK | |
| [SP]- | NIGGDEDDKNIGSDEDHLSLKEFSELEQSGYYVCYPRGSKPEDANFYLYLRARVCENCMEMDVM | |
| [VH1VL1 | SVATIVIVDICITGGLLLLVYYWSKNRKAKAKPVTRGAGAGGRQRGQNKERPPPVPNPDYEPIRK | |
| scFv]-[CD3ϵ | GQRDLYSGLNQRRIGSGEGRGSLLTCGDVEENPGPVSKGEELFTGVVPILVELDGDVNGHKFSVS | |
| ECD]-[CD3ϵ | GEGEGDATYGKLTLKFICTTGKLPVPWPTLVTTLTYGVQCFSRYPDHMKQHDFFKSAMPEGYVQ | |
| TMD]-[CD3ϵ | ERTIFFKDDGNYKTRAEVKFEGDTLVNRIELKGIDFKEDGNILGHKLEYNYNSHNVYIMADKQKN | |
| ICD]-[T2A]- | GIKVNFKIRHNIEDGSVQLADHYQQNTPIGDGPVLLPDNHYLSTQSALSKDPNEKRDHMVLLEFV | |
| [eGFP] | TAAGITLGMDELYK | |
| P329G | EVQLVESGGGLVQPGGSLRLSCAASGFDFSRYWMNWVRQAPGKGLEWVGEITPDSSTINYTPSL | 212 |
| VH1VL1 | KGRFTISRDNAKNSLYLQMNSLRAEDTAVYYCVRPYDYGAWFASWGQGTLVTVSSGGGGSGG | |
| CD3ϵ(with | GGSGGGGSQAVVTQEPSLTVSPGGTVTLTCRSSTGAVTTSNYANWVQEKPDHLFTGLIGGTNKR | |
| T2A, eGFP) | APGTPARFSGSLLGGKAALTLSGAQPEDEAEYYCALWYSNHWVFGGGTKLTVLGGGGSGGGGS | |
| [VH1VL1 | GGGGSDGNEEMGGITQTPYKVSISGTTVILTCPQYPGSEILWQHNDKNIGGDEDDKNIGSDEDHLS | |
| scFv]-[CD3ϵ | LKEFSELEQSGYYVCYPRGSKPEDANFYLYLRARVCENCMEMDVMSVATIVIVDICITGGLLLLV | |
| ECD]-[CD3ϵ | YYWSKNRKAKAKPVTRGAGAGGRQRGQNKERPPPVPNPDYEPIRKGQRDLYSGLNQRRIGSGE | |
| TMD]-[CD3ϵ | GRGSLLTCGDVEENPGPVSKGEELFTGVVPILVELDGDVNGHKFSVSGEGEGDATYGKLTLKFIC | |
| ICD]-[T2A]- | TTGKLPVPWPTLVTTLTYGVQCFSRYPDHMKQHDFFKSAMPEGYVQERTIFFKDDGNYKTRAEV | |
| [eGFP] | KFEGDTLVNRIELKGIDFKEDGNILGHKLEYNYNSHNVYIMADKQKNGIKVNFKIRHNIEDGSVQ | |
| | LADHYQQNTPIGDGPVLLPDNHYLSTQSALSKDPNEKRDHMVLLEFVTAAGITLGMDELYK | |
| P329G | MGWSCIILFLVATATGVHSEVQLVESGGGLVQPGGSLRLSCAASGFDFSRYWMNWVRQAPGKG | 213 |
| VH1VL1 | LEWVGEITPDSSTINYTPSLKGRFTISRDNAKNSLYLQMNSLRAEDTAVYYCVRPYDYGAWFAS | |
| CD3ϵ(with | WGQGTLVTVSSGGGGSGGGGSGGGGSQAVVTQEPSLTVSPGGTVTLTCRSSTGAVTTSNYANW | |
| SP) | VQEKPDHLFTGLIGGTNKRAPGTPARFSGSLLGGKAALTLSGAQPEDEAEYYCALWYSNHWVFG | |
| [SP]- | GGTKLTVLGGGGSGGGGSGGGGSDGNEEMGGITQTPYKVSISGTTVILTCPQYPGSEILWQHNDK | |
| [VH1VL1 | NIGGDEDDKNIGSDEDHLSLKEFSELEQSGYYVCYPRGSKPEDANFYLYLRARVCENCMEMDVM | |
| scFv]-[CD3ϵ | SVATIVIVDICITGGLLLLVYYWSKNRKAKAKPVTRGAGAGGRQRGQNKERPPPVPNPDYEPIRK | |
| ECD]-[CD3ϵ | GQRDLYSGLNQRRI | |
| TMD]-[CD3ϵ | | |
| ICD] | | |
| P329G | EVQLVESGGGLVQPGGSLRLSCAASGFDFSRYWMNWVRQAPGKGLEWVGEITPDSSTINYTPSL | 214 |
| VH1VL1 | KGRFTISRDNAKNSLYLQMNSLRAEDTAVYYCVRPYDYGAWFASWGQGTLVTVSSGGGGSGG | |
| CD3ϵ | GGSGGGGSQAVVTQEPSLTVSPGGTVTLTCRSSTGAVTTSNYANWVQEKPDHLFTGLIGGTNKR | |
| (mature) | APGTPARFSGSLLGGKAALTLSGAQPEDEAEYYCALWYSNHWVFGGGTKLTVLGGGGSGGGGS | |
| [VH1VL1 | GGGGSDGNEEMGGITQTPYKVSISGTTVILTCPQYPGSEILWQHNDKNIGGDEDDKNIGSDEDHLS | |
| scFv]-[CD3ϵ | LKEFSELEQSGYYVCYPRGSKPEDANFYLYLRARVCENCMEMDVMSVATIVIVDICITGGLLLLV | |
| ECD]-[CD3ϵ | YYWSKNRKAKAKPVTRGAGAGGRQRGQNKERPPPVPNPDYEPIRKGQRDLYSGLNQRRI | |
| TMD]-[CD3ϵ | | |
| ICD] | | |
| P329G | MGWSCIILFLVATATGVHSEVQLVESGGGLVQPGGSLRLSCAASGFDFSRYWMNWVRQAPGKG | 215 |
| VH2VL1 | LEWVGEITPDSSTINYAPSLKGRFTISRDNAKNSLYLQMNSLRAEDTAVYYCVRPYDYGAWFAS | |
| CD3ϵ(with | WGQGTLVTVSSGGGGSGGGGSGGGGSQAVVTQEPSLTVSPGGTVTLTCRSSTGAVTTSNYANW | |
| SP, T2A, | VQEKPDHLFTGLIGGTNKRAPGTPARFSGSLLGGKAALTLSGAQPEDEAEYYCALWYSNHWVFG | |
| eGFP) | GGTKLTVLGGGGSGGGGSGGGGSDGNEEMGGITQTPYKVSISGTTVILTCPQYPGSEILWQHNDK | |
| [SP]- | NIGGDEDDKNIGSDEDHLSLKEFSELEQSGYYVCYPRGSKPEDANFYLYLRARVCENCMEMDVM | |
| [VH2VL1 | SVATIVIVDICITGGLLLLVYYWSKNRKAKAKPVTRGAGAGGRQRGQNKERPPPVPNPDYEPIRK | |
| scFv]-[CD3ϵ | GQRDLYSGLNQRRIGSGEGRGSLLTCGDVEENPGPVSKGEELFTGVVPILVELDGDVNGHKFSVS | |
| ECD]-[CD3ϵ | GEGEGDATYGKLTLKFICTTGKLPVPWPTLVTTLTYGVQCFSRYPDHMKQHDFFKSAMPEGYVQ | |
| TMD]-[CD38 | ERTIFFKDDGNYKTRAEVKFEGDTLVNRIELKGIDFKEDGNILGHKLEYNYNSHNVYIMADKQKN | |
| ICD]-[T2A]- | GIKVNFKIRHNIEDGSVQLADHYQQNTPIGDGPVLLPDNHYLSTQSALSKDPNEKRDHMVLLEFV | |
| [eGFP] | TAAGITLGMDELYK | |
| P329G | EVQLVESGGGLVQPGGSLRLSCAASGFDFSRYWMNWVRQAPGKGLEWVGEITPDSSTINYAPSL | 216 |
| VH2VL1 | KGRFTISRDNAKNSLYLQMNSLRAEDTAVYYCVRPYDYGAWFASWGQGTLVTVSSGGGGSGG | |
| CD3ϵ(with | GGSGGGGSQAVVTQEPSLTVSPGGTVTLTCRSSTGAVTTSNYANWVQEKPDHLFTGLIGGTNKR | |
| T2A, eGFP) | APGTPARFSGSLLGGKAALTLSGAQPEDEAEYYCALWYSNHWVFGGGTKLTVLGGGGSGGGGS | |
| [VH2VL1 | GGGGSDGNEEMGGITQTPYKVSISGTTVILTCPQYPGSEILWQHNDKNIGGDEDDKNIGSDEDHLS | |
| scFv]-[CD3ϵ | LKEFSELEQSGYYVCYPRGSKPEDANFYLYLRARVCENCMEMDVMSVATIVIVDICITGGLLLLV | |
| ECD]-[CD3ϵ | YYWSKNRKAKAKPVTRGAGAGGRQRGQNKERPPPVPNPDYEPIRKGQRDLYSGLNQRRIGSGE | |
| TMD]-[CD3ϵ | GRGSLLTCGDVEENPGPVSKGEELFTGVVPILVELDGDVNGHKFSVSGEGEGDATYGKLTLKFIC | |
| ICD]-[T2A]- | TTGKLPVPWPTLVTTLTYGVQCFSRYPDHMKQHDFFKSAMPEGYVQERTIFFKDDGNYKTRAEV | |
| [eGFP] | KFEGDTLVNRIELKGIDFKEDGNILGHKLEYNYNSHNVYIMADKQKNGIKVNFKIRHNIEDGSVQ | |
| | LADHYQQNTPIGDGPVLLPDNHYLSTQSALSKDPNEKRDHMVLLEFVTAAGITLGMDELYK | |
| P329G | MGWSCIILFLVATATGVHSEVQLVESGGGLVQPGGSLRLSCAASGFDFSRYWMNWVRQAPGKG | 217 |
| VH2VL1 | LEWVGEITPDSSTINYAPSLKGRFTISRDNAKNSLYLQMNSLRAEDTAVYYCVRPYDYGAWFAS | |
| CD3ϵ(with | WGQGTLVTVSSGGGGSGGGGSGGGGSQAVVTQEPSLTVSPGGTVTLTCRSSTGAVTTSNYANW | |
| SP) | VQEKPDHLFTGLIGGTNKRAPGTPARFSGSLLGGKAALTLSGAQPEDEAEYYCALWYSNHWVFG | |
| [SP]- | GGTKLTVLGGGGSGGGGSGGGGSDGNEEMGGITQTPYKVSISGTTVILTCPQYPGSEILWQHNDK | |
| [VH2VL1 | NIGGDEDDKNIGSDEDHLSLKEFSELEQSGYYVCYPRGSKPEDANFYLYLRARVCENCMEMDVM | |
| scFv]-[CD3ϵ | SVATIVIVDICITGGLLLLVYYWSKNRKAKAKPVTRGAGAGGRQRGQNKERPPPVPNPDYEPIRK | |
| ECD]-[CD3ϵ | GQRDLYSGLNQRRI | |
| TMD]-[CD3ϵ | | |
| ICD] | | |
| P329G | EVQLVESGGGLVQPGGSLRLSCAASGFDFSRYWMNWVRQAPGKGLEWVGEITPDSSTINYAPSL | 218 |
| VH2VL1 | KGRFTISRDNAKNSLYLQMNSLRAEDTAVYYCVRPYDYGAWFASWGQGTLVTVSSGGGGSGG | |
| CD3ϵ | GGSGGGGSQAVVTQEPSLTVSPGGTVTLTCRSSTGAVTTSNYANWVQEKPDHLFTGLIGGTNKR | |
| (mature) | APGTPARFSGSLLGGKAALTLSGAQPEDEAEYYCALWYSNHWVFGGGTKLTVLGGGGSGGGGS | |
| [VH2VL1 | GGGGSDGNEEMGGITQTPYKVSISGTTVILTCPQYPGSEILWQHNDKNIGGDEDDKNIGSDEDHLS | |
| scFv]-[CD3ϵ | LKEFSELEQSGYYVCYPRGSKPEDANFYLYLRARVCENCMEMDVMSVATIVIVDICITGGLLLLV | |
| ECD]-[CD3ϵ | YYWSKNRKAKAKPVTRGAGAGGRQRGQNKERPPPVPNPDYEPIRKGQRDLYSGLNQRRI | |
| TMD]-[CD3ϵ | | |
| ICD] | | |
| P329G | MGWSCIILFLVATATGVHSEVQLVESGGGLVQPGGSLRLSCAASGFTFSRYWMNWVRQAPGKG | 219 |
| VH3VL1 | LEWVGEITPDSSTINYAPSLKGRFTISRDNAKNSLYLQMNSLRAEDTAVYYCARPYDYGAWFAS | |
| CD3ϵ (with | WGQGTLVTVSSGGGGSGGGGSGGGGSQAVVTQEPSLTVSPGGTVTLTCRSSTGAVTTSNYANW | |
| SP, T2A, | VQEKPDHLFTGLIGGTNKRAPGTPARFSGSLLGGKAALTLSGAQPEDEAEYYCALWYSNHWVFG | |
| eGFP) | GGTKLTVLGGGGSGGGGSGGGGSDGNEEMGGITQTPYKVSISGTTVILTCPQYPGSEILWQHNDK | |
| [SP]- | NIGGDEDDKNIGSDEDHLSLKEFSELEQSGYYVCYPRGSKPEDANFYLYLRARVCENCMEMDVM | |
| [VH3VL1 | SVATIVIVDICITGGLLLLVYYWSKNRKAKAKPVTRGAGAGGRQRGQNKERPPPVPNPDYEPIRK | |
| scFv]-[CD3ϵ | GQRDLYSGLNQRRIGSGEGRGSLLTCGDVEENPGPVSKGEELFTGVVPILVELDGDVNGHKFSVS | |
| ECD]-[CD3ϵ | GEGEGDATYGKLTLKFICTTGKLPVPWPTLVTTLTYGVQCFSRYPDHMKQHDFFKSAMPEGYVQ | |
| TMD]-[CD3ϵ | ERTIFFKDDGNYKTRAEVKFEGDTLVNRIELKGIDFKEDGNILGHKLEYNYNSHNVYIMADKQKN | |
| ICD]-[T2A]- | GIKVNFKIRHNIEDGSVQLADHYQQNTPIGDGPVLLPDNHYLSTQSALSKDPNEKRDHMVLLEFV | |
| [eGFP] | TAAGITLGMDELYK | |
| P329G | EVQLVESGGGLVQPGGSLRLSCAASGFTFSRYWMNWVRQAPGKGLEWVGEITPDSSTINYAPSL | 220 |
| VH3VL1 | KGRFTISRDNAKNSLYLQMNSLRAEDTAVYYCARPYDYGAWFASWGQGTLVTVSSGGGGSGG | |
| CD3ϵ (with | GGSGGGGSQAVVTQEPSLTVSPGGTVTLTCRSSTGAVTTSNYANWVQEKPDHLFTGLIGGTNKR | |
| T2A, eGFP) | APGTPARFSGSLLGGKAALTLSGAQPEDEAEYYCALWYSNHWVFGGGTKLTVLGGGGSGGGGS | |
| [VH3VL1 | GGGGSDGNEEMGGITQTPYKVSISGTTVILTCPQYPGSEILWQHNDKNIGGDEDDKNIGSDEDHLS | |
| scFv]-[CD3ϵ | LKEFSELEQSGYYVCYPRGSKPEDANFYLYLRARVCENCMEMDVMSVATIVIVDICITGGLLLLV | |
| ECD]-[CD3ϵ | YYWSKNRKAKAKPVTRGAGAGGRQRGQNKERPPPVPNPDYEPIRKGQRDLYSGLNQRRIGSGE | |
| TMD]-[CD3ϵ | GRGSLLTCGDVEENPGPVSKGEELFTGVVPILVELDGDVNGHKFSVSGEGEGDATYGKLTLKFIC | |
| ICD]-[T2A]- | TTGKLPVPWPTLVTTLTYGVQCFSRYPDHMKQHDFFKSAMPEGYVQERTIFFKDDGNYKTRAEV | |
| [eGFP] | KFEGDTLVNRIELKGIDFKEDGNILGHKLEYNYNSHNVYIMADKQKNGIKVNFKIRHNIEDGSVQ | |
| | LADHYQQNTPIGDGPVLLPDNHYLSTQSALSKDPNEKRDHMVLLEFVTAAGITLGMDELYK | |
| P329G | MGWSCIILFLVATATGVHSEVQLVESGGGLVQPGGSLRLSCAASGFTFSRYWMNWVRQAPGKG | 221 |
| VH3VL1 | LEWVGEITPDSSTINYAPSLKGRFTISRDNAKNSLYLQMNSLRAEDTAVYYCARPYDYGAWFAS | |
| CD3ϵ(with | WGQGTLVTVSSGGGGSGGGGSGGGGSQAVVTQEPSLTVSPGGTVTLTCRSSTGAVTTSNYANW | |
| SP) | VQEKPDHLFTGLIGGTNKRAPGTPARFSGSLLGGKAALTLSGAQPEDEAEYYCALWYSNHWVFG | |
| [SP]- | GGTKLTVLGGGGSGGGGSGGGGSDGNEEMGGITQTPYKVSISGTTVILTCPQYPGSEILWQHNDK | |
| [VH3VL1 | NIGGDEDDKNIGSDEDHLSLKEFSELEQSGYYVCYPRGSKPEDANFYLYLRARVCENCMEMDVM | |
| scFv]-[CD3ϵ | SVATIVIVDICITGGLLLLVYYWSKNRKAKAKPVTRGAGAGGRQRGQNKERPPPVPNPDYEPIRK | |
| ECD]-[CD3ϵ | GQRDLYSGLNQRRI | |
| TMD]-[CD3ϵ | | |
| ICD] | | |
| P329G | EVQLVESGGGLVQPGGSLRLSCAASGFTFSRYWMNWVRQAPGKGLEWVGEITPDSSTINYAPSL | 222 |
| VH3VL1 | KGRFTISRDNAKNSLYLQMNSLRAEDTAVYYCARPYDYGAWFASWGQGTLVTVSSGGGGSGG | |
| CD3ϵ | GGSGGGGSQAVVTQEPSLTVSPGGTVTLTCRSSTGAVTTSNYANWVQEKPDHLFTGLIGGTNKR | |
| (mature) | APGTPARFSGSLLGGKAALTLSGAQPEDEAEYYCALWYSNHWVFGGGTKLTVLGGGGSGGGGS | |
| [VH3VL1 | GGGGSDGNEEMGGITQTPYKVSISGTTVILTCPQYPGSEILWQHNDKNIGGDEDDKNIGSDEDHLS | |
| scFv]-[CD3ϵ | LKEFSELEQSGYYVCYPRGSKPEDANFYLYLRARVCENCMEMDVMSVATIVIVDICITGGLLLLV | |
| ECD]-[CD3ϵ | YYWSKNRKAKAKPVTRGAGAGGRQRGQNKERPPPVPNPDYEPIRKGQRDLYSGLNQRRI | |
| TMD]-[CD3ϵ | | |
| ICD] | | |
| SP-VH3VL1- | MGWSCIILFLVATATGVHSEVQLVESGGGLVQPGGSLRLSCAASGFTFSRYWMNWVRQAPGKG | 223 |
| CD3ϵ ECD- | LEWVGEITPDSSTINYAPSLKGRFTISRDNAKNSLYLQMNSLRAEDTAVYYCARPYDYGAWFAS | |
| CD3ϵ TMD- | WGQGTLVTVSSGGGGSGGGGSGGGGSQAVVTQEPSLTVSPGGTVTLTCRSSTGAVTTSNYANW | |
| CD3ϵ ICD- | VQEKPDHLFTGLIGGTNKRAPGTPARFSGSLLGGKAALTLSGAQPEDEAEYYCALWYSNHWVFG | |
| E2A-eGFP | GGTKLTVLGGGGSGGGGSGGGGSDGNEEMGGITQTPYKVSISGTTVILTCPQYPGSEILWQHNDK | |
| | NIGGDEDDKNIGSDEDHLSLKEFSELEQSGYYVCYPRGSKPEDANFYLYLRARVCENCMEMDVM | |
| | SVATIVIVDICITGGLLLLVYYWSKNRKAKAKPVTRGAGAGGRQRGQNKERPPPVPNPDYEPIRK | |
| | GQRDLYSGLNQRRIGSGQCTNYALLKLAGDVESNPGPVSKGEELFTGVVPILVELDGDVNGHKFS | |
| | VSGEGEGDATYGKLTLKFICTTGKLPVPWPTLVTTLTYGVQCFSRYPDHMKQHDFFKSAMPEGY | |
| | VQERTIFFKDDGNYKTRAEVKFEGDTLVNRIELKGIDFKEDGNILGHKLEYNYNSHNVYIMADKQ | |
| | KNGIKVNFKIRHNIEDGSVQLADHYQQNTPIGDGPVLLPDNHYLSTQSALSKDPNEKRDHMVLLE | |
| | FVTAAGITLGMDELYK | |
| P329G | MGWSCIILFLVATATGVHSEVQLVESGGGLVQPGGSLRLSCAASGFDFSRYWMNWVRQAPGKG | 224 |
| TCRαß- | LEWVGEITPDSSTINYTPSLKGRFTISRDNAKNSLYLQMNSLRAEDTAVYYCVRPYDYGAWFAS | |
| VH1TRAC | WGQGTLVTVSSIQNPDPAVYQLRDSKSSDKSVCLFTDFDSQTNVSQSKDSDVYITDKCVLDMRS | |
| (with SP, | MDFKSNSAVAWSNKSDFACANAFNNSIIPEDTFFPSPESSCDVKLVEKSFETDTNLNFQNLSVIGF | |
| T2A, eGFP) | RILLLKVAGFNLLMTLRLWSSGSGEGRGSLLTCGDVEENPGPVSKGEELFTGVVPILVELDGDVN | |
| [SP]-[VH1]- | GHKFSVSGEGEGDATYGKLTLKFICTTGKLPVPWPTLVTTLTYGVQCFSRYPDHMKQHDFFKSA | |
| [TRAC_T47C]- | MPEGYVQERTIFFKDDGNYKTRAEVKFEGDTLVNRIELKGIDFKEDGNILGHKLEYNYNSHNVYI | |
| ][T2A]- | MADKQKNGIKVNFKIRHNIEDGSVQLADHYQQNTPIGDGPVLLPDNHYLSTQSALSKDPNEKRD | |
| [eGFP] | HMVLLEFVTAAGITLGMDELYK | |
| P329G | EVQLVESGGGLVQPGGSLRLSCAASGFDFSRYWMNWVRQAPGKGLEWVGEITPDSSTINYTPSL | 225 |
| TCRαß- | KGRFTISRDNAKNSLYLQMNSLRAEDTAVYYCVRPYDYGAWFASWGQGTLVTVSSIQNPDPAV | |
| VH1TRAC | YQLRDSKSSDKSVCLFTDFDSQTNVSQSKDSDVYITDKCVLDMRSMDFKSNSAVAWSNKSDFAC | |
| (with T2A, | ANAFNNSIIPEDTFFPSPESSCDVKLVEKSFETDTNLNFQNLSVIGFRILLLKVAGFNLLMTLRLWS | |
| eGFP) | SGSGEGRGSLLTCGDVEENPGPVSKGEELFTGVVPILVELDGDVNGHKFSVSGEGEGDATYGKLT | |
| [VH1]- | LKFICTTGKLPVPWPTLVTTLTYGVQCFSRYPDHMKQHDFFKSAMPEGYVQERTIFFKDDGNYK | |
| [TRAC_T47C]- | TRAEVKFEGDTLVNRIELKGIDFKEDGNILGHKLEYNYNSHNVYIMADKQKNGIKVNFKIRHNIE | |
| ][T2A]- | DGSVQLADHYQQNTPIGDGPVLLPDNHYLSTQSALSKDPNEKRDHMVLLEFVTAAGITLGMDEL | |
| [eGFP] | YK | |
| P329G | MGWSCIILFLVATATGVHSEVQLVESGGGLVQPGGSLRLSCAASGFDFSRYWMNWVRQAPGKG | 226 |
| TCRαß- | LEWVGEITPDSSTINYTPSLKGRFTISRDNAKNSLYLQMNSLRAEDTAVYYCVRPYDYGAWFAS | |
| VH1TRAC | WGQGTLVTVSSIQNPDPAVYQLRDSKSSDKSVCLFTDFDSQTNVSQSKDSDVYITDKCVLDMRS | |
| (with SP) | MDFKSNSAVAWSNKSDFACANAFNNSIIPEDTFFPSPESSCDVKLVEKSFETDTNLNFQNLSVIGF | |
| [SP]-[VH1]- | RILLLKVAGFNLLMTLRLWSS | |
| [TRAC_T47C] | | |
| P329G | EVQLVESGGGLVQPGGSLRLSCAASGFDFSRYWMNWVRQAPGKGLEWVGEITPDSSTINYTPSL | 227 |
| TCRαß- | KGRFTISRDNAKNSLYLQMNSLRAEDTAVYYCVRPYDYGAWFASWGQGTLVTVSSIQNPDPAV | |
| VH1TRAC | YQLRDSKSSDKSVCLFTDFDSQTNVSQSKDSDVYITDKCVLDMRSMDFKSNSAVAWSNKSDFAC | |
| (mature) | ANAFNNSIIPEDTFFPSPESSCDVKLVEKSFETDTNLNFQNLSVIGFRILLLKVAGFNLLMTLRLWS | |
| [VH1]- | S | |
| [TRAC_T47C] | | |
| P329G | MGWSCIILFLVATATGVHSEVQLVESGGGLVQPGGSLRLSCAASGFDFSRYWMNWVRQAPGKG | 228 |
| TCRαß- | LEWVGEITPDSSTINYAPSLKGRFTISRDNAKNSLYLQMNSLRAEDTAVYYCVRPYDYGAWFAS | |
| VH2TRAC | WGQGTLVTVSSIQNPDPAVYQLRDSKSSDKSVCLFTDFDSQTNVSQSKDSDVYITDKCVLDMRS | |
| (with SP, | MDFKSNSAVAWSNKSDFACANAFNNSIIPEDTFFPSPESSCDVKLVEKSFETDTNLNFQNLSVIGF | |
| T2A, eGFP) | RILLLKVAGFNLLMTLRLWSSGSGEGRGSLLTCGDVEENPGPVSKGEELFTGVVPILVELDGDVN | |
| [SP]-[VH2]- | GHKFSVSGEGEGDATYGKLTLKFICTTGKLPVPWPTLVTTLTYGVQCFSRYPDHMKQHDFFKSA | |
| [TRAC_T47C]- | MPEGYVQERTIFFKDDGNYKTRAEVKFEGDTLVNRIELKGIDFKEDGNILGHKLEYNYNSHNVYI | |
| ][T2A]- | MADKQKNGIKVNFKIRHNIEDGSVQLADHYQQNTPIGDGPVLLPDNHYLSTQSALSKDPNEKRD | |
| [eGFP] | HMVLLEFVTAAGITLGMDELYK | |
| P329G | EVQLVESGGGLVQPGGSLRLSCAASGFDFSRYWMNWVRQAPGKGLEWVGEITPDSSTINYAPSL | 229 |
| TCRαß- | KGRFTISRDNAKNSLYLQMNSLRAEDTAVYYCVRPYDYGAWFASWGQGTLVTVSSIQNPDPAV | |
| VH2TRAC | YQLRDSKSSDKSVCLFTDFDSQTNVSQSKDSDVYITDKCVLDMRSMDFKSNSAVAWSNKSDFAC | |
| (with T2A, | ANAFNNSIIPEDTFFPSPESSCDVKLVEKSFETDTNLNFQNLSVIGFRILLLKVAGFNLLMTLRLWS | |
| eGFP) | SGSGEGRGSLLTCGDVEENPGPVSKGEELFTGVVPILVELDGDVNGHKFSVSGEGEGDATYGKLT | |
| [VH2]- | LKFICTTGKLPVPWPTLVTTLTYGVQCFSRYPDHMKQHDFFKSAMPEGYVQERTIFFKDDGNYK | |
| [TRAC T47C]- | TRAEVKFEGDTLVNRIELKGIDFKEDGNILGHKLEYNYNSHNVYIMADKQKNGIKVNFKIRHNIE | |
| ][T2A]- | DGSVQLADHYQQNTPIGDGPVLLPDNHYLSTQSALSKDPNEKRDHMVLLEFVTAAGITLGMDEL | |
| [eGFP] | YK | |
| P329G | MGWSCIILFLVATATGVHSEVQLVESGGGLVQPGGSLRLSCAASGFDFSRYWMNWVRQAPGKG | 230 |
| TCRαß- | LEWVGEITPDSSTINYAPSLKGRFTISRDNAKNSLYLQMNSLRAEDTAVYYCVRPYDYGAWFAS | |
| VH2TRAC | WGQGTLVTVSSIQNPDPAVYQLRDSKSSDKSVCLFTDFDSQTNVSQSKDSDVYITDKCVLDMRS | |
| (with SP) | MDFKSNSAVAWSNKSDFACANAFNNSIIPEDTFFPSPESSCDVKLVEKSFETDTNLNFQNLSVIGF | |
| [SP]-[VH2]- | RILLLKVAGFNLLMTLRLWSS | |
| [TRAC_T47C] | | |
| P329G | EVQLVESGGGLVQPGGSLRLSCAASGFDFSRYWMNWVRQAPGKGLEWVGEITPDSSTINYAPSL | 231 |
| TCRαß- | KGRFTISRDNAKNSLYLQMNSLRAEDTAVYYCVRPYDYGAWFASWGQGTLVTVSSIQNPDPAV | |
| VH2TRAC | YQLRDSKSSDKSVCLFTDFDSQTNVSQSKDSDVYITDKCVLDMRSMDFKSNSAVAWSNKSDFAC | |
| (mature) | ANAFNNSIIPEDTFFPSPESSCDVKLVEKSFETDTNLNFQNLSVIGFRILLLKVAGFNLLMTLRLWS | |
| [VH2]- | S | |
| [TRAC T47 | | |
| C] | | |
| P329G | MGWSCIILFLVATATGVHSEVQLVESGGGLVQPGGSLRLSCAASGFTFSRYWMNWVRQAPGKG | 232 |
| TCRαß- | LEWVGEITPDSSTINYAPSLKGRFTISRDNAKNSLYLQMNSLRAEDTAVYYCARPYDYGAWFAS | |
| VH3TRAC | WGQGTLVTVSSIQNPDPAVYQLRDSKSSDKSVCLFTDFDSQTNVSQSKDSDVYITDKCVLDMRS | |
| (with SP, | MDFKSNSAVAWSNKSDFACANAFNNSIIPEDTFFPSPESSCDVKLVEKSFETDTNLNFQNLSVIGF | |
| T2A, eGFP) | RILLLKVAGFNLLMTLRLWSSGSGEGRGSLLTCGDVEENPGPVSKGEELFTGVVPILVELDGDVN | |
| [SP]-[VH3]- | GHKFSVSGEGEGDATYGKLTLKFICTTGKLPVPWPTLVTTLTYGVQCFSRYPDHMKQHDFFKSA | |
| [TRAC_T47 | MPEGYVQERTIFFKDDGNYKTRAEVKFEGDTLVNRIELKGIDFKEDGNILGHKLEYNYNSHNVYI | |
| C]-[T2A]- | MADKQKNGIKVNFKIRHNIEDGSVQLADHYQQNTPIGDGPVLLPDNHYLSTQSALSKDPNEKRD | |
| [eGFP] | HMVLLEFVTAAGITLGMDELYK | |
| P329G | EVQLVESGGGLVQPGGSLRLSCAASGFTFSRYWMNWVRQAPGKGLEWVGEITPDSSTINYAPSL | 233 |
| TCRαß | KGRFTISRDNAKNSLYLQMNSLRAEDTAVYYCARPYDYGAWFASWGQGTLVTVSSIQNPDPAV | |
| VH3TRAC | YQLRDSKSSDKSVCLFTDFDSQTNVSQSKDSDVYITDKCVLDMRSMDFKSNSAVAWSNKSDFAC | |
| (with T2A, | ANAFNNSIIPEDTFFPSPESSCDVKLVEKSFETDTNLNFQNLSVIGFRILLLKVAGFNLLMTLRLWS | |
| eGFP) | SGSGEGRGSLLTCGDVEENPGPVSKGEELFTGVVPILVELDGDVNGHKFSVSGEGEGDATYGKLT | |
| [VH3]- | LKFICTTGKLPVPWPTLVTTLTYGVQCFSRYPDHMKQHDFFKSAMPEGYVQERTIFFKDDGNYK | |
| [TRAC_T47 | TRAEVKFEGDTLVNRIELKGIDFKEDGNILGHKLEYNYNSHNVYIMADKQKNGIKVNFKIRHNIE | |
| C]-[T2A]- | DGSVQLADHYQQNTPIGDGPVLLPDNHYLSTQSALSKDPNEKRDHMVLLEFVTAAGITLGMDEL | |
| [eGFP] | YK | |
| P329G | MGWSCIILFLVATATGVHSEVQLVESGGGLVQPGGSLRLSCAASGFTFSRYWMNWVRQAPGKG | 234 |
| TCRαß- | LEWVGEITPDSSTINYAPSLKGRFTISRDNAKNSLYLQMNSLRAEDTAVYYCARPYDYGAWFAS | |
| VH3TRAC | WGQGTLVTVSSIQNPDPAVYQLRDSKSSDKSVCLFTDFDSQTNVSQSKDSDVYITDKCVLDMRS | |
| (with SP) | MDFKSNSAVAWSNKSDFACANAFNNSIIPEDTFFPSPESSCDVKLVEKSFETDTNLNFQNLSVIGF | |
| [SP]-[VH3]- | RILLLKVAGFNLLMTLRLWSS | |
| [TRAC_T47C] | | |
| P329G | EVQLVESGGGLVQPGGSLRLSCAASGFTFSRYWMNWVRQAPGKGLEWVGEITPDSSTINYAPSL | 235 |
| TCRαß- | KGRFTISRDNAKNSLYLQMNSLRAEDTAVYYCARPYDYGAWFASWGQGTLVTVSSIQNPDPAV | |
| VH3TRAC | YQLRDSKSSDKSVCLFTDFDSQTNVSQSKDSDVYITDKCVLDMRSMDFKSNSAVAWSNKSDFAC | |
| (mature) | ANAFNNSIIPEDTFFPSPESSCDVKLVEKSFETDTNLNFQNLSVIGFRILLLKVAGFNLLMTLRLWS | |
| [VH3]- | S | |
| [TRAC_T47C] | | |
| P329G | MGWSCIILFLVATATGVHSQAVVTQEPSLTVSPGGTVTLTCRSSTGAVTTSNYANWVQEKPDHLF | 236 |
| TCRαß- | TGLIGGTNKRAPGTPARFSGSLLGGKAALTLSGAQPEDEAEYYCALWYSNHWVFGGGTKLTVLI | |
| VL1TRAC | QNPDPAVYQLRDSKSSDKSVCLFTDFDSQTNVSQSKDSDVYITDKCVLDMRSMDFKSNSAVAWS | |
| (with SP, | NKSDFACANAFNNSIIPEDTFFPSPESSCDVKLVEKSFETDTNLNFQNLSVIGFRILLLKVAGENLL | |
| T2A, eGFP) | MTLRLWSSGSGEGRGSLLTCGDVEENPGPVSKGEELFTGVVPILVELDGDVNGHKFSVSGEGEG | |
| [SP]-[VL1]- | DATYGKLTLKFICTTGKLPVPWPTLVTTLTYGVQCFSRYPDHMKQHDFFKSAMPEGYVQERTIFF | |
| [TRAC T47C]- | KDDGNYKTRAEVKFEGDTLVNRIELKGIDFKEDGNILGHKLEYNYNSHNVYIMADKQKNGIKVN | |
| ][T2A]- | FKIRHNIEDGSVQLADHYQQNTPIGDGPVLLPDNHYLSTQSALSKDPNEKRDHMVLLEFVTAAGI | |
| [eGFP] | TLGMDELYK | |
| P329G | QAVVTQEPSLTVSPGGTVTLTCRSSTGAVTTSNYANWVQEKPDHLFTGLIGGTNKRAPGTPARFS | 237 |
| TCRαß- | GSLLGGKAALTLSGAQPEDEAEYYCALWYSNHWVFGGGTKLTVLIQNPDPAVYQLRDSKSSDK | |
| VL1TRAC | SVCLFTDFDSQTNVSQSKDSDVYITDKCVLDMRSMDFKSNSAVAWSNKSDFACANAFNNSIIPED | |
| (with T2A, | TFFPSPESSCDVKLVEKSFETDTNLNFQNLSVIGFRILLLKVAGFNLLMTLRLWSSGSGEGRGSLLT | |
| eGFP) | CGDVEENPGPVSKGEELFTGVVPILVELDGDVNGHKFSVSGEGEGDATYGKLTLKFICTTGKLPV | |
| [VL1]- | PWPTLVTTLTYGVQCFSRYPDHMKQHDFFKSAMPEGYVQERTIFFKDDGNYKTRAEVKFEGDTL | |
| [TRAC_T47C]- | VNRIELKGIDFKEDGNILGHKLEYNYNSHNVYIMADKQKNGIKVNFKIRHNIEDGSVQLADHYQQ | |
| ][T2A]- | NTPIGDGPVLLPDNHYLSTQSALSKDPNEKRDHMVLLEFVTAAGITLGMDELYK | |
| [eGFP] | | |
| P329G | MGWSCIILFLVATATGVHSQAVVTQEPSLTVSPGGTVTLTCRSSTGAVTTSNYANWVQEKPDHLF | 238 |
| TCRαß- | TGLIGGTNKRAPGTPARFSGSLLGGKAALTLSGAQPEDEAEYYCALWYSNHWVFGGGTKLTVLI | |
| VL1TRAC | QNPDPAVYQLRDSKSSDKSVCLFTDFDSQTNVSQSKDSDVYITDKCVLDMRSMDFKSNSAVAWS | |
| (with SP) | NKSDFACANAFNNSIIPEDTFFPSPESSCDVKLVEKSFETDTNLNFQNLSVIGFRILLLKVAGFNLL | |
| [SP]-[VL1]- | MTLRLWSS | |
| [TRAC_T47C] | | |
| P329G | QAVVTQEPSLTVSPGGTVTLTCRSSTGAVTTSNYANWVQEKPDHLFTGLIGGTNKRAPGTPARFS | 239 |
| TCRαß- | GSLLGGKAALTLSGAQPEDEAEYYCALWYSNHWVFGGGTKLTVLIQNPDPAVYQLRDSKSSDK | |
| VLITRAC | SVCLFTDFDSQTNVSQSKDSDVYITDKCVLDMRSMDFKSNSAVAWSNKSDFACANAFNNSIIPED | |
| (mature) | TFFPSPESSCDVKLVEKSFETDTNLNFQNLSVIGFRILLLKVAGFNLLMTLRLWSS | |
| [VL1]- | | |
| [TRAC_T47C] | | |
| P329G | MGWSCIILFLVATATGVHSEVQLVESGGGLVQPGGSLRLSCAASGFDFSRYWMNWVRQAPGKG | 240 |
| TCRαß- | LEWVGEITPDSSTINYTPSLKGRFTISRDNAKNSLYLQMNSLRAEDTAVYYCVRPYDYGAWFAS | |
| VH1TRBC1 | WGQGTLVTVSSDLNKVFPPEVAVFEPSEAEISHTQKATLVCLATGFFPDHVELSWWVNGKEVHS | |
| (with SP, | GVCTDPQPLKEQPALNDSRYCLSSRLRVSATFWQNPRNHFRCQVQFYGLSENDEWTQDRAKPVT | |
| T2A, eGFP) | QIVSAEAWGRADCGFTSVSYQQGVLSATILYEILLGKATLYAVLVSALVLMAMVKRKDFGSGEG | |
| [SP]-[VH1]- | RGSLLTCGDVEENPGPVSKGEELFTGVVPILVELDGDVNGHKFSVSGEGEGDATYGKLTLKFICT | |
| [TRBC1_S56C]- | TGKLPVPWPTLVTTLTYGVQCFSRYPDHMKQHDFFKSAMPEGYVQERTIFFKDDGNYKTRAEVK | |
| ][T2A]- | FEGDTLVNRIELKGIDFKEDGNILGHKLEYNYNSHNVYIMADKQKNGIKVNFKIRHNIEDGSVQL | |
| [eGFP] | ADHYQQNTPIGDGPVLLPDNHYLSTQSALSKDPNEKRDHMVLLEFVTAAGITLGMDELYK | |
| P329G | EVQLVESGGGLVQPGGSLRLSCAASGFDFSRYWMNWVRQAPGKGLEWVGEITPDSSTINYTPSL | 241 |
| TCRαß- | KGRFTISRDNAKNSLYLQMNSLRAEDTAVYYCVRPYDYGAWFASWGQGTLVTVSSDLNKVFPP | |
| VH1TRBC1 | EVAVFEPSEAEISHTQKATLVCLATGFFPDHVELSWWVNGKEVHSGVCTDPQPLKEQPALNDSR | |
| (with T2A, | YCLSSRLRVSATFWQNPRNHFRCQVQFYGLSENDEWTQDRAKPVTQIVSAEAWGRADCGFTSVS | |
| eGFP) | YQQGVLSATILYEILLGKATLYAVLVSALVLMAMVKRKDFGSGEGRGSLLTCGDVEENPGPVSK | |
| [VH1]- | GEELFTGVVPILVELDGDVNGHKFSVSGEGEGDATYGKLTLKFICTTGKLPVPWPTLVTTLTYGV | |
| [TRBC1_S56C]- | QCFSRYPDHMKQHDFFKSAMPEGYVQERTIFFKDDGNYKTRAEVKFEGDTLVNRIELKGIDFKE | |
| ][T2A]- | DGNILGHKLEYNYNSHNVYIMADKQKNGIKVNFKIRHNIEDGSVQLADHYQQNTPIGDGPVLLP | |
| [eGFP] | DNHYLSTQSALSKDPNEKRDHMVLLEFVTAAGITLGMDELYK | |
| P329G | MGWSCIILFLVATATGVHSEVQLVESGGGLVQPGGSLRLSCAASGFDFSRYWMNWVRQAPGKG | 242 |
| TCRαß- | LEWVGEITPDSSTINYTPSLKGRFTISRDNAKNSLYLQMNSLRAEDTAVYYCVRPYDYGAWFAS | |
| VH1TRBC1 | WGQGTLVTVSSDLNKVFPPEVAVFEPSEAEISHTQKATLVCLATGFFPDHVELSWWVNGKEVHS | |
| (with SP) | GVCTDPQPLKEQPALNDSRYCLSSRLRVSATFWQNPRNHFRCQVQFYGLSENDEWTQDRAKPVT | |
| [SP]-[VH1]- | QIVSAEAWGRADCGFTSVSYQQGVLSATILYEILLGKATLYAVLVSALVLMAMVKRKDF | |
| [TRBC1_S56C] | | |
| P329G | EVQLVESGGGLVQPGGSLRLSCAASGFDFSRYWMNWVRQAPGKGLEWVGEITPDSSTINYTPSL | 243 |
| TCRαß- | KGRFTISRDNAKNSLYLQMNSLRAEDTAVYYCVRPYDYGAWFASWGQGTLVTVSSDLNKVFPP | |
| VH1TRBC1 | EVAVFEPSEAEISHTQKATLVCLATGFFPDHVELSWWVNGKEVHSGVCTDPQPLKEQPALNDSR | |
| (mature) | YCLSSRLRVSATFWQNPRNHFRCQVQFYGLSENDEWTQDRAKPVTQIVSAEAWGRADCGFTSVS | |
| [VH1]- | YQQGVLSATILYEILLGKATLYAVLVSALVLMAMVKRKDF | |
| [TRBC1_S56C] | | |

]

| | | |
|---|---|---|
| P329G | MGWSCIILFLVATATGVHSEVQLVESGGGLVQPGGSLRLSCAASGFDFSRYWMNWVRQAPGKG | 244 |
| TCRαß- | LEWVGEITPDSSTINYAPSLKGRFTISRDNAKNSLYLQMNSLRAEDTAVYYCVRPYDYGAWFAS | |
| VH2TRBC1 | WGQGTLVTVSSDLNKVFPPEVAVFEPSEAEISHTQKATLVCLATGFFPDHVELSWWVNGKEVHS | |
| (with SP, | GVCTDPQPLKEQPALNDSRYCLSSRLRVSATFWQNPRNHFRCQVQFYGLSENDEWTQDRAKPVT | |
| T2A, eGFP) | QIVSAEAWGRADCGFTSVSYQQGVLSATILYEILLGKATLYAVLVSALVLMAMVKRKDFGSGEG | |
| [SP]-[VH2]- | RGSLLTCGDVEENPGPVSKGEELFTGVVPILVELDGDVNGHKFSVSGEGEGDATYGKLTLKFICT | |
| [TRBC1_S56C]- | TGKLPVPWPTLVTTLTYGVQCFSRYPDHMKQHDFFKSAMPEGYVQERTIFFKDDGNYKTRAEVK | |
| ][T2A]- | FEGDTLVNRIELKGIDFKEDGNILGHKLEYNYNSHNVYIMADKQKNGIKVNFKIRHNIEDGSVQL | |
| [eGFP] | ADHYQQNTPIGDGPVLLPDNHYLSTQSALSKDPNEKRDHMVLLEFVTAAGITLGMDELYK | |
| P329G | EVQLVESGGGLVQPGGSLRLSCAASGFDFSRYWMNWVRQAPGKGLEWVGEITPDSSTINYAPSL | 245 |
| TCRαß- | KGRFTISRDNAKNSLYLQMNSLRAEDTAVYYCVRPYDYGAWFASWGQGTLVTVSSDLNKVFPP | |
| VH2TRBC1 | EVAVFEPSEAEISHTQKATLVCLATGFFPDHVELSWWVNGKEVHSGVCTDPQPLKEQPALNDSR | |
| (with T2A, | YCLSSRLRVSATFWQNPRNHFRCQVQFYGLSENDEWTQDRAKPVTQIVSAEAWGRADCGFTSVS | |
| eGFP) | YQQGVLSATILYEILLGKATLYAVLVSALVLMAMVKRKDFGSGEGRGSLLTCGDVEENPGPVSK | |
| [VH2]- | GEELFTGVVPILVELDGDVNGHKFSVSGEGEGDATYGKLTLKFICTTGKLPVPWPTLVTTLTYGV | |
| [TRBC1_S56C]- | QCFSRYPDHMKQHDFFKSAMPEGYVQERTIFFKDDGNYKTRAEVKFEGDTLVNRIELKGIDFKE | |
| ][T2A]- | DGNILGHKLEYNYNSHNVYIMADKQKNGIKVNFKIRHNIEDGSVQLADHYQQNTPIGDGPVLLP | |
| [eGFP] | DNHYLSTQSALSKDPNEKRDHMVLLEFVTAAGITLGMDELYK | |
| P329G | MGWSCIILFLVATATGVHSEVQLVESGGGLVQPGGSLRLSCAASGFDFSRYWMNWVRQAPGKG | 246 |
| TCRαß- | LEWVGEITPDSSTINYAPSLKGRFTISRDNAKNSLYLQMNSLRAEDTAVYYCVRPYDYGAWFAS | |
| VH2TRBC1 | WGQGTLVTVSSDLNKVFPPEVAVFEPSEAEISHTQKATLVCLATGFFPDHVELSWWVNGKEVHS | |
| (with SP) | GVCTDPQPLKEQPALNDSRYCLSSRLRVSATFWQNPRNHFRCQVQFYGLSENDEWTQDRAKPVT | |
| [SP]-[VH2]- | QIVSAEAWGRADCGFTSVSYQQGVLSATILYEILLGKATLYAVLVSALVLMAMVKRKDF | |
| [TRBC1_S56C] | | |

]

| | | |
|---|---|---|
| P329G | EVQLVESGGGLVQPGGSLRLSCAASGFDFSRYWMNWVRQAPGKGLEWVGEITPDSSTINYAPSL | 247 |
| TCRαß- | KGRFTISRDNAKNSLYLQMNSLRAEDTAVYYCVRPYDYGAWFASWGQGTLVTVSSDLNKVFPP | |
| VH2TRBC1 | EVAVFEPSEAEISHTQKATLVCLATGFFPDHVELSWWVNGKEVHSGVCTDPQPLKEQPALNDSR | |
| (mature) | YCLSSRLRVSATFWQNPRNHFRCQVQFYGLSENDEWTQDRAKPVTQIVSAEAWGRADCGFTSVS | |
| [VH2]- | YQQGVLSATILYEILLGKATLYAVLVSALVLMAMVKRKDF | |
| [TRBC1_S56C] | | |
| P329G | MGWSCIILFLVATATGVHSEVQLVESGGGLVQPGGSLRLSCAASGFTFSRYWMNWVRQAPGKG | 248 |
| TCRαß- | LEWVGEITPDSSTINYAPSLKGRFTISRDNAKNSLYLQMNSLRAEDTAVYYCARPYDYGAWFAS | |
| VH3TRBC1 | WGQGTLVTVSSDLNKVFPPEVAVFEPSEAEISHTQKATLVCLATGFFPDHVELSWWVNGKEVHS | |
| (with SP, | GVCTDPQPLKEQPALNDSRYCLSSRLRVSATFWQNPRNHFRCQVQFYGLSENDEWTQDRAKPVT | |
| T2A, eGFP) | QIVSAEAWGRADCGFTSVSYQQGVLSATILYEILLGKATLYAVLVSALVLMAMVKRKDFGSGEG | |
| [SP]-[VH3]- | RGSLLTCGDVEENPGPVSKGEELFTGVVPILVELDGDVNGHKFSVSGEGEGDATYGKLTLKFICT | |
| [TRBC1_S56C]- | TGKLPVPWPTLVTTLTYGVQCFSRYPDHMKQHDFFKSAMPEGYVQERTIFFKDDGNYKTRAEVK | |
| ][T2A]- | FEGDTLVNRIELKGIDFKEDGNILGHKLEYNYNSHNVYIMADKQKNGIKVNFKIRHNIEDGSVQL | |
| [eGFP] | ADHYQQNTPIGDGPVLLPDNHYLSTQSALSKDPNEKRDHMVLLEFVTAAGITLGMDELYK | |
| P329G | EVQLVESGGGLVQPGGSLRLSCAASGFTFSRYWMNWVRQAPGKGLEWVGEITPDSSTINYAPSL | 249 |
| TCRαß- | KGRFTISRDNAKNSLYLQMNSLRAEDTAVYYCARPYDYGAWFASWGQGTLVTVSSDLNKVFPP | |
| VH3TRBC1 | EVAVFEPSEAEISHTQKATLVCLATGFFPDHVELSWWVNGKEVHSGVCTDPQPLKEQPALNDSR | |
| (with T2A, | YCLSSRLRVSATFWQNPRNHFRCQVQFYGLSENDEWTQDRAKPVTQIVSAEAWGRADCGFTSVS | |
| eGFP) | YQQGVLSATILYEILLGKATLYAVLVSALVLMAMVKRKDFGSGEGRGSLLTCGDVEENPGPVSK | |
| [VH3]- | GEELFTGVVPILVELDGDVNGHKFSVSGEGEGDATYGKLTLKFICTTGKLPVPWPTLVTTLTYGV | |
| [TRBC1_S56C]- | QCFSRYPDHMKQHDFFKSAMPEGYVQERTIFFKDDGNYKTRAEVKFEGDTLVNRIELKGIDFKE | |
| ][T2A]- | DGNILGHKLEYNYNSHNVYIMADKQKNGIKVNFKIRHNIEDGSVQLADHYQQNTPIGDGPVLLP | |
| [eGFP] | DNHYLSTQSALSKDPNEKRDHMVLLEFVTAAGITLGMDELYK | |
| P329G | MGWSCIILFLVATATGVHSEVQLVESGGGLVQPGGSLRLSCAASGFTFSRYWMNWVRQAPGKG | 250 |
| TCRαß- | LEWVGEITPDSSTINYAPSLKGRFTISRDNAKNSLYLQMNSLRAEDTAVYYCARPYDYGAWFAS | |
| VH3TRBC1 | WGQGTLVTVSSDLNKVFPPEVAVFEPSEAEISHTQKATLVCLATGFFPDHVELSWWVNGKEVHS | |
| (with SP) | GVCTDPQPLKEQPALNDSRYCLSSRLRVSATFWQNPRNHFRCQVQFYGLSENDEWTQDRAKPVT | |
| [SP]-[VH3] | QIVSAEAWGRADCGFTSVSYQQGVLSATILYEILLGKATLYAVLVSALVLMAMVKRKDF | |
| [TRBC1_S56C] | | |

]

| | | |
|---|---|---|
| P329G | EVQLVESGGGLVQPGGSLRLSCAASGFTFSRYWMNWVRQAPGKGLEWVGEITPDSSTINYAPSL | 251 |
| TCRαß- | KGRFTISRDNAKNSLYLQMNSLRAEDTAVYYCARPYDYGAWFASWGQGTLVTVSSDLNKVFPP | |
| VH3TRBC1 | EVAVFEPSEAEISHTQKATLVCLATGFFPDHVELSWWVNGKEVHSGVCTDPQPLKEQPALNDSR | |
| (mature) | YCLSSRLRVSATFWQNPRNHFRCQVQFYGLSENDEWTQDRAKPVTQIVSAEAWGRADCGFTSVS | |
| [VH3]- | YQQGVLSATILYEILLGKATLYAVLVSALVLMAMVKRKDF | |
| [TRBC1_S56C] | | |

]

| | | |
|---|---|---|
| P329G | MGWSCIILFLVATATGVHSQAVVTQEPSLTVSPGGTVTLTCRSSTGAVTTSNYANWVQEKPDHLF | 252 |
| TCRαß- | TGLIGGTNKRAPGTPARFSGSLLGGKAALTLSGAQPEDEAEYYCALWYSNHWVFGGGTKLTVLD | |
| VL1TRBC1 | LNKVFPPEVAVFEPSEAEISHTQKATLVCLATGFFPDHVELSWWVNGKEVHSGVCTDPQPLKEQP | |
| (with SP, | ALNDSRYCLSSRLRVSATFWQNPRNHFRCQVQFYGLSENDEWTQDRAKPVTQIVSAEAWGRAD | |
| T2A, eGFP) | CGFTSVSYQQGVLSATILYEILLGKATLYAVLVSALVLMAMVKRKDFGSGEGRGSLLTCGDVEE | |
| [SP]-[VL1]- | NPGPVSKGEELFTGVVPILVELDGDVNGHKFSVSGEGEGDATYGKLTLKFICTTGKLPVPWPTLV | |
| [TRBC1_S56C]- | TTLTYGVQCFSRYPDHMKQHDFFKSAMPEGYVQERTIFFKDDGNYKTRAEVKFEGDTLVNRIEL | |
| ][T2A]- | KGIDFKEDGNILGHKLEYNYNSHNVYIMADKQKNGIKVNFKIRHNIEDGSVQLADHYQQNTPIGD | |
| [eGFP] | GPVLLPDNHYLSTQSALSKDPNEKRDHMVLLEFVTAAGITLGMDELYK | |
| P329G | QAVVTQEPSLTVSPGGTVTLTCRSSTGAVTTSNYANWVQEKPDHLFTGLIGGTNKRAPGTPARFS | 253 |
| TCRαß- | GSLLGGKAALTLSGAQPEDEAEYYCALWYSNHWVFGGGTKLTVLDLNKVFPPEVAVFEPSEAEI | |
| VL1TRBC1 | SHTQKATLVCLATGFFPDHVELSWWVNGKEVHSGVCTDPQPLKEQPALNDSRYCLSSRLRVSAT | |
| (with T2A, | FWQNPRNHFRCQVQFYGLSENDEWTQDRAKPVTQIVSAEAWGRADCGFTSVSYQQGVLSATIL | |
| eGFP) | YEILLGKATLYAVLVSALVLMAMVKRKDFGSGEGRGSLLTCGDVEENPGPVSKGEELFTGVVPIL | |
| [VL1]- | VELDGDVNGHKFSVSGEGEGDATYGKLTLKFICTTGKLPVPWPTLVTTLTYGVQCFSRYPDHMK | |
| [TRBC1_S56C]- | QHDFFKSAMPEGYVQERTIFFKDDGNYKTRAEVKFEGDTLVNRIELKGIDFKEDGNILGHKLEYN | |
| ][T2A]- | YNSHNVYIMADKQKNGIKVNFKIRHNIEDGSVQLADHYQQNTPIGDGPVLLPDNHYLSTQSALSK | |
| [eGFP] | DPNEKRDHMVLLEFVTAAGITLGMDELYK | |
| P329G | MGWSCIILFLVATATGVHSQAVVTQEPSLTVSPGGTVTLTCRSSTGAVTTSNYANWVQEKPDHLF | 254 |
| TCRαß- | TGLIGGTNKRAPGTPARFSGSLLGGKAALTLSGAQPEDEAEYYCALWYSNHWVFGGGTKLTVLD | |
| VL1TRBC1 | LNKVFPPEVAVFEPSEAEISHTQKATLVCLATGFFPDHVELSWWVNGKEVHSGVCTDPQPLKEQP | |
| (with SP) | ALNDSRYCLSSRLRVSATFWQNPRNHFRCQVQFYGLSENDEWTQDRAKPVTQIVSAEAWGRAD | |
| [SP]-[VL1]- | CGFTSVSYQQGVLSATILYEILLGKATLYAVLVSALVLMAMVKRKDF | |
| [TRBC1_S56C] | | |

]

| | | |
|---|---|---|
| P329G | QAVVTQEPSLTVSPGGTVTLTCRSSTGAVTTSNYANWVQEKPDHLFTGLIGGTNKRAPGTPARFS | 255 |
| TCRαß- | GSLLGGKAALTLSGAQPEDEAEYYCALWYSNHWVFGGGTKLTVLDLNKVFPPEVAVFEPSEAEI | |
| VL1TRBC1 | SHTQKATLVCLATGFFPDHVELSWWVNGKEVHSGVCTDPQPLKEQPALNDSRYCLSSRLRVSAT | |
| (mature) | FWQNPRNHFRCQVQFYGLSENDEWTQDRAKPVTQIVSAEAWGRADCGFTSVSYQQGVLSATIL | |
| [VL1]- | YEILLGKATLYAVLVSALVLMAMVKRKDF | |
| [TRBC1_S56C] | | |

]

| | | |
|---|---|---|
| P329G | MGWSCIILFLVATATGVHSEVQLVESGGGLVQPGGSLRLSCAASGFDFSRYWMNWVRQAPGKG | 256 |
| VH1VL1 | LEWVGEITPDSSTINYTPSLKGRFTISRDNAKNSLYLQMNSLRAEDTAVYYCVRPYDYGAWFAS | |
| TCRαß (with | WGQGTLVTVSSIQNPDPAVYQLRDSKSSDKSVCLFTDFDSQTNVSQSKDSDVYITDKCVLDMRS | |
| SP, T2A, | MDFKSNSAVAWSNKSDFACANAFNNSIIPEDTFFPSPESSCDVKLVEKSFETDTNLNFQNLSVIGF | |
| eGFP) | RILLLKVAGFNLLMTLRLWSSGSGEGRGSLLTCGDVEENPGPMGWSCIILFLVATATGVHSQAVV | |
| [SP]-[VH1]- | TQEPSLTVSPGGTVTLTCRSSTGAVTTSNYANWVQEKPDHLFTGLIGGTNKRAPGTPARFSGSLL | |
| [TRAC_T47C]- | GGKAALTLSGAQPEDEAEYYCALWYSNHWVFGGGTKLTVLDLNKVFPPEVAVFEPSEAEISHTQ | |
| ][T2A]- | KATLVCLATGFFPDHVELSWWVNGKEVHSGVCTDPQPLKEQPALNDSRYCLSSRLRVSATFWQ | |
| [SP]-[VL1]- | NPRNHFRCQVQFYGLSENDEWTQDRAKPVTQIVSAEAWGRADCGFTSVSYQQGVLSATILYEIL | |
| [TRBC1_S56C]- | LGKATLYAVLVSALVLMAMVKRKDFGSGEGRGSLLTCGDVEENPGPVSKGEELFTGVVPILVEL | |
| ][T2A]- | DGDVNGHKFSVSGEGEGDATYGKLTLKFICTTGKLPVPWPTLVTTLTYGVQCFSRYPDHMKQHD | |
| [eGFP] | FFKSAMPEGYVQERTIFFKDDGNYKTRAEVKFEGDTLVNRIELKGIDFKEDGNILGHKLEYNYNS | |
| | HNVYIMADKQKNGIKVNFKIRHNIEDGSVQLADHYQQNTPIGDGPVLLPDNHYLSTQSALSKDPN | |
| | EKRDHMVLLEFVTAAGITLGMDELYK | |
| P329G | MGWSCIILFLVATATGVHSEVQLVESGGGLVQPGGSLRLSCAASGFDFSRYWMNWVRQAPGKG | 257 |
| VH1VL1 | LEWVGEITPDSSTINYTPSLKGRFTISRDNAKNSLYLQMNSLRAEDTAVYYCVRPYDYGAWFAS | |
| TCRαß (with | WGQGTLVTVSSIQNPDPAVYQLRDSKSSDKSVCLFTDFDSQTNVSQSKDSDVYITDKCVLDMRS | |
| SP) | MDFKSNSAVAWSNKSDFACANAFNNSIIPEDTFFPSPESSCDVKLVEKSFETDTNLNFQNLSVIGF | |
| [SP]-[VH1]- | RILLLKVAGFNLLMTLRLWSSGSGEGRGSLLTCGDVEENPGPMGWSCIILFLVATATGVHSQAVV | |
| [TRAC_T47C]- | TQEPSLTVSPGGTVTLTCRSSTGAVTTSNYANWVQEKPDHLFTGLIGGTNKRAPGTPARFSGSLL | |
| ][T2A]- | GGKAALTLSGAQPEDEAEYYCALWYSNHWVFGGGTKLTVLDLNKVFPPEVAVFEPSEAEISHTQ | |
| [SP]-[VL1]- | KATLVCLATGFFPDHVELSWWVNGKEVHSGVCTDPQPLKEQPALNDSRYCLSSRLRVSATFWQ | |
| [TRBC1_S56C] | NPRNHFRCQVQFYGLSENDEWTQDRAKPVTQIVSAEAWGRADCGFTSVSYQQGVLSATILYEIL | |
| ] | LGKATLYAVLVSALVLMAMVKRKDF | |
| P329G | MGWSCIILFLVATATGVHSEVQLVESGGGLVQPGGSLRLSCAASGFDFSRYWMNWVRQAPGKG | 258 |
| VH2VL1 | LEWVGEITPDSSTINYAPSLKGRFTISRDNAKNSLYLQMNSLRAEDTAVYYCVRPYDYGAWFAS | |
| TCRαß (with | WGQGTLVTVSSIQNPDPAVYQLRDSKSSDKSVCLFTDFDSQTNVSQSKDSDVYITDKCVLDMRS | |
| SP, T2A, | MDFKSNSAVAWSNKSDFACANAFNNSIIPEDTFFPSPESSCDVKLVEKSFETDTNLNFQNLSVIGF | |
| eGFP) | RILLLKVAGFNLLMTLRLWSSGSGEGRGSLLTCGDVEENPGPMGWSCIILFLVATATGVHSQAVV | |
| [SP]-[VH2]- | TQEPSLTVSPGGTVTLTCRSSTGAVTTSNYANWVQEKPDHLFTGLIGGTNKRAPGTPARFSGSLL | |
| [TRAC_T47C]- | GGKAALTLSGAQPEDEAEYYCALWYSNHWVFGGGTKLTVLDLNKVFPPEVAVFEPSEAEISHTQ | |
| ][T2A]- | KATLVCLATGFFPDHVELSWWVNGKEVHSGVCTDPQPLKEQPALNDSRYCLSSRLRVSATFWQ | |
| [SP]-[VL1]- | NPRNHFRCQVQFYGLSENDEWTQDRAKPVTQIVSAEAWGRADCGFTSVSYQQGVLSATILYEIL | |
| [TRBC1_S56C]- | LGKATLYAVLVSALVLMAMVKRKDFGSGEGRGSLLTCGDVEENPGPVSKGEELFTGVVPILVEL | |
| ][T2A]- | DGDVNGHKFSVSGEGEGDATYGKLTLKFICTTGKLPVPWPTLVTTLTYGVQCFSRYPDHMKQHD | |
| [eGFP] | FFKSAMPEGYVQERTIFFKDDGNYKTRAEVKFEGDTLVNRIELKGIDFKEDGNILGHKLEYNYNS | |
| | HNVYIMADKQKNGIKVNFKIRHNIEDGSVQLADHYQQNTPIGDGPVLLPDNHYLSTQSALSKDPN | |
| | EKRDHMVLLEFVTAAGITLGMDELYK | |
| P329G | MGWSCIILFLVATATGVHSEVQLVESGGGLVQPGGSLRLSCAASGFDFSRYWMNWVRQAPGKG | 259 |
| VH2VL1 | LEWVGEITPDSSTINYAPSLKGRFTISRDNAKNSLYLQMNSLRAEDTAVYYCVRPYDYGAWFAS | |
| TCRαß (with | WGQGTLVTVSSIQNPDPAVYQLRDSKSSDKSVCLFTDFDSQTNVSQSKDSDVYITDKCVLDMRS | |
| SP) | MDFKSNSAVAWSNKSDFACANAFNNSIIPEDTFFPSPESSCDVKLVEKSFETDTNLNFQNLSVIGF | |
| [SP]-[VH2]- | RILLLKVAGFNLLMTLRLWSSGSGEGRGSLLTCGDVEENPGPMGWSCIILFLVATATGVHSQAVV | |
| [TRAC_T47C]- | TQEPSLTVSPGGTVTLTCRSSTGAVTTSNYANWVQEKPDHLFTGLIGGTNKRAPGTPARFSGSLL | |
| ][T2A]- | GGKAALTLSGAQPEDEAEYYCALWYSNHWVFGGGTKLTVLDLNKVFPPEVAVFEPSEAEISHTQ | |
| [SP]-[VL1]- | KATLVCLATGFFPDHVELSWWVNGKEVHSGVCTDPQPLKEQPALNDSRYCLSSRLRVSATFWQ | |
| [TRBC1_S56C] | NPRNHFRCQVQFYGLSENDEWTQDRAKPVTQIVSAEAWGRADCGFTSVSYQQGVLSATILYEIL | |
| ] | LGKATLYAVLVSALVLMAMVKRKDF | |
| P329G | MGWSCIILFLVATATGVHSEVQLVESGGGLVQPGGSLRLSCAASGFTFSRYWMNWVRQAPGKG | 260 |
| VH3VL1 | LEWVGEITPDSSTINYAPSLKGRFTISRDNAKNSLYLQMNSLRAEDTAVYYCARPYDYGAWFAS | |
| TCRαß (with | WGQGTLVTVSSIQNPDPAVYQLRDSKSSDKSVCLFTDFDSQTNVSQSKDSDVYITDKCVLDMRS | |
| SP, T2A, | MDFKSNSAVAWSNKSDFACANAFNNSIIPEDTFFPSPESSCDVKLVEKSFETDTNLNFQNLSVIGF | |
| eGFP) | RILLLKVAGFNLLMTLRLWSSGSGEGRGSLLTCGDVEENPGPMGWSCIILFLVATATGVHSQAVV | |
| [SP]-[VH3]- | TQEPSLTVSPGGTVTLTCRSSTGAVTTSNYANWVQEKPDHLFTGLIGGTNKRAPGTPARFSGSLL | |
| [TRAC_T47C]- | GGKAALTLSGAQPEDEAEYYCALWYSNHWVFGGGTKLTVLDLNKVFPPEVAVFEPSEAEISHTQ | |
| ][T2A]- | KATLVCLATGFFPDHVELSWWVNGKEVHSGVCTDPQPLKEQPALNDSRYCLSSRLRVSATFWQ | |
| [SP]-[VL1]- | NPRNHFRCQVQFYGLSENDEWTQDRAKPVTQIVSAEAWGRADCGFTSVSYQQGVLSATILYEIL | |
| [TRBC1_S56C]- | LGKATLYAVLVSALVLMAMVKRKDFGSGEGRGSLLTCGDVEENPGPVSKGEELFTGVVPILVEL | |
| ][T2A]- | DGDVNGHKFSVSGEGEGDATYGKLTLKFICTTGKLPVPWPTLVTTLTYGVQCFSRYPDHMKQHD | |
| [eGFP] | FFKSAMPEGYVQERTIFFKDDGNYKTRAEVKFEGDTLVNRIELKGIDFKEDGNILGHKLEYNYNS | |
| | HNVYIMADKQKNGIKVNFKIRHNIEDGSVQLADHYQQNTPIGDGPVLLPDNHYLSTQSALSKDPN | |
| | EKRDHMVLLEFVTAAGITLGMDELYK | |
| P329G | MGWSCIILFLVATATGVHSEVQLVESGGGLVQPGGSLRLSCAASGFTFSRYWMNWVRQAPGKG | 261 |
| VH3VL1 | LEWVGEITPDSSTINYAPSLKGRFTISRDNAKNSLYLQMNSLRAEDTAVYYCARPYDYGAWFAS | |
| TCRαß (with | WGQGTLVTVSSIQNPDPAVYQLRDSKSSDKSVCLFTDFDSQTNVSQSKDSDVYITDKCVLDMRS | |
| SP) | MDFKSNSAVAWSNKSDFACANAFNNSIIPEDTFFPSPESSCDVKLVEKSFETDTNLNFQNLSVIGF | |
| [SP]-[VH3]- | RILLLKVAGFNLLMTLRLWSSGSGEGRGSLLTCGDVEENPGPMGWSCIILFLVATATGVHSQAVV | |
| [TRAC_T47C]- | TQEPSLTVSPGGTVTLTCRSSTGAVTTSNYANWVQEKPDHLFTGLIGGTNKRAPGTPARFSGSLL | |
| ][T2A]- | GGKAALTLSGAQPEDEAEYYCALWYSNHWVFGGGTKLTVLDLNKVFPPEVAVFEPSEAEISHTQ | |
| [SP]-[VL1]- | KATLVCLATGFFPDHVELSWWVNGKEVHSGVCTDPQPLKEQPALNDSRYCLSSRLRVSATFWQ | |
| [TRBC1_S56C] | NPRNHFRCQVQFYGLSENDEWTQDRAKPVTQIVSAEAWGRADCGFTSVSYQQGVLSATILYEIL | |
| ] | LGKATLYAVLVSALVLMAMVKRKDF | |
| P329G | MGWSCIILFLVATATGVHSEVQLVESGGGLVQPGGSLRLSCAASGFDFSRYWMNWVRQAPGKG | 262 |
| VH1VL1 | LEWVGEITPDSSTINYTPSLKGRFTISRDNAKNSLYLQMNSLRAEDTAVYYCVRPYDYGAWFAS | |
| TCRαß (with | WGQGTLVTVSSDLNKVFPPEVAVFEPSEAEISHTQKATLVCLATGFFPDHVELSWWVNGKEVHS | |
| SP, T2A, | GVCTDPQPLKEQPALNDSRYCLSSRLRVSATFWQNPRNHFRCQVQFYGLSENDEWTQDRAKPVT | |
| eGFP) | QIVSAEAWGRADCGFTSVSYQQGVLSATILYEILLGKATLYAVLVSALVLMAMVKRKDFGSGEG | |
| [SP]-[VH1]- | RGSLLTCGDVEENPGPMGWSCIILFLVATATGVHSQAVVTQEPSLTVSPGGTVTLTCRSSTGAVT | |
| [TRBC1_S56C]- | TSNYANWVQEKPDHLFTGLIGGTNKRAPGTPARFSGSLLGGKAALTLSGAQPEDEAEYYCALWY | |
| ][T2A]- | SNHWVFGGGTKLTVLIQNPDPAVYQLRDSKSSDKSVCLFTDFDSQTNVSQSKDSDVYITDKCVL | |
| [SP]-[VL1]- | DMRSMDFKSNSAVAWSNKSDFACANAFNNSIIPEDTFFPSPESSCDVKLVEKSFETDTNLNFQNLS | |
| [TRAC_T47C]- | VIGFRILLLKVAGFNLLMTLRLWSSGSGEGRGSLLTCGDVEENPGPVSKGEELFTGVVPILVELDG | |
| ][T2A]- | DVNGHKFSVSGEGEGDATYGKLTLKFICTTGKLPVPWPTLVTTLTYGVQCFSRYPDHMKQHDFF | |
| [eGFP] | KSAMPEGYVQERTIFFKDDGNYKTRAEVKFEGDTLVNRIELKGIDFKEDGNILGHKLEYNYNSHN | |
| | VYIMADKQKNGIKVNFKIRHNIEDGSVQLADHYQQNTPIGDGPVLLPDNHYLSTQSALSKDPNEK | |
| | RDHMVLLEFVTAAGITLGMDELYK | |
| P329G | MGWSCIILFLVATATGVHSEVQLVESGGGLVQPGGSLRLSCAASGFDFSRYWMNWVRQAPGKG | 263 |
| VH1VL1 | LEWVGEITPDSSTINYTPSLKGRFTISRDNAKNSLYLQMNSLRAEDTAVYYCVRPYDYGAWFAS | |
| TCRαß (with | WGQGTLVTVSSDLNKVFPPEVAVFEPSEAEISHTQKATLVCLATGFFPDHVELSWWVNGKEVHS | |
| SP) | GVCTDPQPLKEQPALNDSRYCLSSRLRVSATFWQNPRNHFRCQVQFYGLSENDEWTQDRAKPVT | |
| [SP]-[VH1]- | QIVSAEAWGRADCGFTSVSYQQGVLSATILYEILLGKATLYAVLVSALVLMAMVKRKDFGSGEG | |
| [TRBC1_S56C]- | RGSLLTCGDVEENPGPMGWSCIILFLVATATGVHSQAVVTQEPSLTVSPGGTVTLTCRSSTGAVT | |
| ][T2A]- | TSNYANWVQEKPDHLFTGLIGGTNKRAPGTPARFSGSLLGGKAALTLSGAQPEDEAEYYCALWY | |
| [SP]-[VL1]- | SNHWVFGGGTKLTVLIQNPDPAVYQLRDSKSSDKSVCLFTDFDSQTNVSQSKDSDVYITDKCVL | |
| [TRAC_T47C] | DMRSMDFKSNSAVAWSNKSDFACANAFNNSIIPEDTFFPSPESSCDVKLVEKSFETDTNLNFQNLS | |
| ] | VIGFRILLLKVAGFNLLMTLRLWSS | |
| P329G | MGWSCIILFLVATATGVHSEVQLVESGGGLVQPGGSLRLSCAASGFDFSRYWMNWVRQAPGKG | 264 |
| VH2VL1 | LEWVGEITPDSSTINYAPSLKGRFTISRDNAKNSLYLQMNSLRAEDTAVYYCVRPYDYGAWFAS | |
| TCRαß (with | WGQGTLVTVSSDLNKVFPPEVAVFEPSEAEISHTQKATLVCLATGFFPDHVELSWWVNGKEVHS | |
| SP, T2A, | GVCTDPQPLKEQPALNDSRYCLSSRLRVSATFWQNPRNHFRCQVQFYGLSENDEWTQDRAKPVT | |
| eGFP) | QIVSAEAWGRADCGFTSVSYQQGVLSATILYEILLGKATLYAVLVSALVLMAMVKRKDFGSGEG | |
| [SP]-[VH2]- | RGSLLTCGDVEENPGPMGWSCIILFLVATATGVHSQAVVTQEPSLTVSPGGTVTLTCRSSTGAVT | |
| [TRBC1_S56C]- | TSNYANWVQEKPDHLFTGLIGGTNKRAPGTPARFSGSLLGGKAALTLSGAQPEDEAEYYCALWY | |
| ][T2A]- | SNHWVFGGGTKLTVLIQNPDPAVYQLRDSKSSDKSVCLFTDFDSQTNVSQSKDSDVYITDKCVL | |
| [SP]-[VL1]- | DMRSMDFKSNSAVAWSNKSDFACANAFNNSIIPEDTFFPSPESSCDVKLVEKSFETDTNLNFQNLS | |
| [TRAC_T47C]- | VIGFRILLLKVAGFNLLMTLRLWSSGSGEGRGSLLTCGDVEENPGPVSKGEELFTGVVPILVELDG | |
| ][T2A]- | DVNGHKFSVSGEGEGDATYGKLTLKFICTTGKLPVPWPTLVTTLTYGVQCFSRYPDHMKQHDFF | |
| [eGFP] | KSAMPEGYVQERTIFFKDDGNYKTRAEVKFEGDTLVNRIELKGIDFKEDGNILGHKLEYNYNSHN | |
| | VYIMADKQKNGIKVNFKIRHNIEDGSVQLADHYQQNTPIGDGPVLLPDNHYLSTQSALSKDPNEK | |
| | RDHMVLLEFVTAAGITLGMDELYK | |
| P329G | MGWSCIILFLVATATGVHSEVQLVESGGGLVQPGGSLRLSCAASGFDFSRYWMNWVRQAPGKG | 265 |
| VH2VL1 | LEWVGEITPDSSTINYAPSLKGRFTISRDNAKNSLYLQMNSLRAEDTAVYYCVRPYDYGAWFAS | |
| TCRαß (with | WGQGTLVTVSSDLNKVFPPEVAVFEPSEAEISHTQKATLVCLATGFFPDHVELSWWVNGKEVHS | |
| SP) | GVCTDPQPLKEQPALNDSRYCLSSRLRVSATFWQNPRNHFRCQVQFYGLSENDEWTQDRAKPVT | |
| [SP]-[VH2]- | QIVSAEAWGRADCGFTSVSYQQGVLSATILYEILLGKATLYAVLVSALVLMAMVKRKDFGSGEG | |
| [TRBC1_S56C]- | RGSLLTCGDVEENPGPMGWSCIILFLVATATGVHSQAVVTQEPSLTVSPGGTVTLTCRSSTGAVT | |
| ][T2A]- | TSNYANWVQEKPDHLFTGLIGGTNKRAPGTPARFSGSLLGGKAALTLSGAQPEDEAEYYCALWY | |
| [SP]-[VL1]- | SNHWVFGGGTKLTVLIQNPDPAVYQLRDSKSSDKSVCLFTDFDSQTNVSQSKDSDVYITDKCVL | |
| [TRAC_T47C] | DMRSMDFKSNSAVAWSNKSDFACANAFNNSIIPEDTFFPSPESSCDVKLVEKSFETDTNLNFQNLS | |
| ] | VIGFRILLLKVAGFNLLMTLRLWSS | |
| P329G | MGWSCIILFLVATATGVHSEVQLVESGGGLVQPGGSLRLSCAASGFTFSRYWMNWVRQAPGKG | 266 |
| VH3VL1 | LEWVGEITPDSSTINYAPSLKGRFTISRDNAKNSLYLQMNSLRAEDTAVYYCARPYDYGAWFAS | |
| TCRαß (with | WGQGTLVTVSSDLNKVFPPEVAVFEPSEAEISHTQKATLVCLATGFFPDHVELSWWVNGKEVHS | |
| SP, T2A, | GVCTDPQPLKEQPALNDSRYCLSSRLRVSATFWQNPRNHFRCQVQFYGLSENDEWTQDRAKPVT | |
| eGFP) | QIVSAEAWGRADCGFTSVSYQQGVLSATILYEILLGKATLYAVLVSALVLMAMVKRKDFGSGEG | |
| [SP]-[VH3]- | RGSLLTCGDVEENPGPMGWSCIILFLVATATGVHSQAVVTQEPSLTVSPGGTVTLTCRSSTGAVT | |
| [TRBC1_S56C]- | TSNYANWVQEKPDHLFTGLIGGTNKRAPGTPARFSGSLLGGKAALTLSGAQPEDEAEYYCALWY | |
| ][T2A]- | SNHWVFGGGTKLTVLIQNPDPAVYQLRDSKSSDKSVCLFTDFDSQTNVSQSKDSDVYITDKCVL | |
| [SP]-[VL1]- | DMRSMDFKSNSAVAWSNKSDFACANAFNNSIIPEDTFFPSPESSCDVKLVEKSFETDTNLNFQNLS | |
| [TRAC_T47C]- | VIGFRILLLKVAGFNLLMTLRLWSSGSGEGRGSLLTCGDVEENPGPVSKGEELFTGVVPILVELDG | |
| ][T2A]- | DVNGHKFSVSGEGEGDATYGKLTLKFICTTGKLPVPWPTLVTTLTYGVQCFSRYPDHMKQHDFF | |
| [eGFP] | KSAMPEGYVQERTIFFKDDGNYKTRAEVKFEGDTLVNRIELKGIDFKEDGNILGHKLEYNYNSHN | |
| | VYIMADKQKNGIKVNFKIRHNIEDGSVQLADHYQQNTPIGDGPVLLPDNHYLSTQSALSKDPNEK | |
| | RDHMVLLEFVTAAGITLGMDELYK | |
| P329G | MGWSCIILFLVATATGVHSEVQLVESGGGLVQPGGSLRLSCAASGFTFSRYWMNWVRQAPGKG | 267 |
| VH3VL1 | LEWVGEITPDSSTINYAPSLKGRFTISRDNAKNSLYLQMNSLRAEDTAVYYCARPYDYGAWFAS | |
| TCRαß (with | WGQGTLVTVSSDLNKVFPPEVAVFEPSEAEISHTQKATLVCLATGFFPDHVELSWWVNGKEVHS | |
| SP) | GVCTDPQPLKEQPALNDSRYCLSSRLRVSATFWQNPRNHFRCQVQFYGLSENDEWTQDRAKPVT | |
| [SP]-[VH3]- | QIVSAEAWGRADCGFTSVSYQQGVLSATILYEILLGKATLYAVLVSALVLMAMVKRKDFGSGEG | |
| [TRBC1_S56C]- | RGSLLTCGDVEENPGPMGWSCIILFLVATATGVHSQAVVTQEPSLTVSPGGTVTLTCRSSTGAVT | |
| ][T2A]- | TSNYANWVQEKPDHLFTGLIGGTNKRAPGTPARFSGSLLGGKAALTLSGAQPEDEAEYYCALWY | |
| [SP]-[VL1]- | SNHWVFGGGTKLTVLIQNPDPAVYQLRDSKSSDKSVCLFTDFDSQTNVSQSKDSDVYITDKCVL | |
| [TRAC_T47C] | DMRSMDFKSNSAVAWSNKSDFACANAFNNSIIPEDTFFPSPESSCDVKLVEKSFETDTNLNFQNLS | |
| ] | VIGFRILLLKVAGFNLLMTLRLWSS | |
| P329G | MGWSCIILFLVATATGVHSQAVVTQEPSLTVSPGGTVTLTCRSSTGAVTTSNYANWVQEKPDHLF | 268 |
| VH1VL1 | TGLIGGTNKRAPGTPARFSGSLLGGKAALTLSGAQPEDEAEYYCALWYSNHWVFGGGTKLTVLI | |
| TCRαß (with | QNPDPAVYQLRDSKSSDKSVCLFTDFDSQTNVSQSKDSDVYITDKCVLDMRSMDFKSNSAVAWS | |
| SP, T2A, | NKSDFACANAFNNSIIPEDTFFPSPESSCDVKLVEKSFETDTNLNFQNLSVIGFRILLLKVAGFNLL | |
| eGFP) | MTLRLWSSGSGEGRGSLLTCGDVEENPGPMGWSCIILFLVATATGVHSDVLMTQTPLSLPVSLGD | |
| [SP]-[VL1]- | QASISCRSSQTIVHSEVQLVESGGGLVQPGGSLRLSCAASGFDFSRYWMNWVRQAPGKGLEWVG | |
| [TRAC_T47C]- | EITPDSSTINYTPSLKGRFTISRDNAKNSLYLQMNSLRAEDTAVYYCVRPYDYGAWFASWGQGTL | |
| C]-[T2A]- | VTVSSDLNKVFPPEVAVFEPSEAEISHTQKATLVCLATGFFPDHVELSWWVNGKEVHSGVCTDPQ | |
| [SP]-[VH1]- | PLKEQPALNDSRYCLSSRLRVSATFWQNPRNHFRCQVQFYGLSENDEWTQDRAKPVTQIVSAEA | |
| [TRBC1_S56C]- | WGRADCGFTSVSYQQGVLSATILYEILLGKATLYAVLVSALVLMAMVKRKDFGSGEGRGSLLTC | |
| ][T2A]- | GDVEENPGPVSKGEELFTGVVPILVELDGDVNGHKFSVSGEGEGDATYGKLTLKFICTTGKLPVP | |
| [eGFP] | WPTLVTTLTYGVQCFSRYPDHMKQHDFFKSAMPEGYVQERTIFFKDDGNYKTRAEVKFEGDTL | |
| | VNRIELKGIDFKEDGNILGHKLEYNYNSHNVYIMADKQKNGIKVNFKIRHNIEDGSVQLADHYQQ | |
| | NTPIGDGPVLLPDNHYLSTQSALSKDPNEKRDHMVLLEFVTAAGITLGMDELYK | |
| P329G | MGWSCIILFLVATATGVHSQAVVTQEPSLTVSPGGTVTLTCRSSTGAVTTSNYANWVQEKPDHLF | 269 |
| VH1VL1 | TGLIGGTNKRAPGTPARFSGSLLGGKAALTLSGAQPEDEAEYYCALWYSNHWVFGGGTKLTVLI | |
| TCRαß (with | QNPDPAVYQLRDSKSSDKSVCLFTDFDSQTNVSQSKDSDVYITDKCVLDMRSMDFKSNSAVAWS | |
| SP) | NKSDFACANAFNNSIIPEDTFFPSPESSCDVKLVEKSFETDTNLNFQNLSVIGFRILLLKVAGFNLL | |
| [SP]-[VL1]- | MTLRLWSSGSGEGRGSLLTCGDVEENPGPMGWSCIILFLVATATGVHSDVLMTQTPLSLPVSLGD | |
| [TRAC T47 | QASISCRSSQTIVHSEVQLVESGGGLVQPGGSLRLSCAASGFDFSRYWMNWVRQAPGKGLEWVG | |
| C]-[T2A]- | EITPDSSTINYTPSLKGRFTISRDNAKNSLYLQMNSLRAEDTAVYYCVRPYDYGAWFASWGQGTL | |
| [SP]-[VH1]- | VTVSSDLNKVFPPEVAVFEPSEAEISHTQKATLVCLATGFFPDHVELSWWVNGKEVHSGVCTDPQ | |
| [TRBC1 S56 | PLKEQPALNDSRYCLSSRLRVSATFWQNPRNHFRCQVQFYGLSENDEWTQDRAKPVTQIVSAEA | |
| C] | WGRADCGFTSVSYQQGVLSATILYEILLGKATLYAVLVSALVLMAMVKRKDF | |
| P329G | MGWSCIILFLVATATGVHSQAVVTQEPSLTVSPGGTVTLTCRSSTGAVTTSNYANWVQEKPDHLF | 270 |
| VH2VL1 | TGLIGGTNKRAPGTPARFSGSLLGGKAALTLSGAQPEDEAEYYCALWYSNHWVFGGGTKLTVLI | |
| TCRαß (with | QNPDPAVYQLRDSKSSDKSVCLFTDFDSQTNVSQSKDSDVYITDKCVLDMRSMDFKSNSAVAWS | |
| SP, T2A, | NKSDFACANAFNNSIIPEDTFFPSPESSCDVKLVEKSFETDTNLNFQNLSVIGFRILLLKVAGENLL | |
| eGFP) | MTLRLWSSGSGEGRGSLLTCGDVEENPGPMGWSCIILFLVATATGVHSDVLMTQTPLSLPVSLGD | |
| [SP]-[VL1]- | QASISCRSSQTIVHSEVQLVESGGGLVQPGGSLRLSCAASGFDFSRYWMNWVRQAPGKGLEWVG | |
| [TRAC_T47C]- | EITPDSSTINYAPSLKGRFTISRDNAKNSLYLQMNSLRAEDTAVYYCVRPYDYGAWFASWGQGT | |
| ][T2A]- | LVTVSSDLNKVFPPEVAVFEPSEAEISHTQKATLVCLATGFFPDHVELSWWVNGKEVHSGVCTDP | |
| [SP]-[VH2]- | QPLKEQPALNDSRYCLSSRLRVSATFWQNPRNHFRCQVQFYGLSENDEWTQDRAKPVTQIVSAE | |
| [TRBC1_S56C]- | AWGRADCGFTSVSYQQGVLSATILYEILLGKATLYAVLVSALVLMAMVKRKDFGSGEGRGSLLT | |
| ][T2A]- | CGDVEENPGPVSKGEELFTGVVPILVELDGDVNGHKFSVSGEGEGDATYGKLTLKFICTTGKLPV | |
| [eGFP] | PWPTLVTTLTYGVQCFSRYPDHMKQHDFFKSAMPEGYVQERTIFFKDDGNYKTRAEVKFEGDTL | |
| | VNRIELKGIDFKEDGNILGHKLEYNYNSHNVYIMADKQKNGIKVNFKIRHNIEDGSVQLADHYQQ | |
| | NTPIGDGPVLLPDNHYLSTQSALSKDPNEKRDHMVLLEFVTAAGITLGMDELYK | |
| P329G | MGWSCIILFLVATATGVHSQAVVTQEPSLTVSPGGTVTLTCRSSTGAVTTSNYANWVQEKPDHLF | 271 |
| VH2VL1 | TGLIGGTNKRAPGTPARFSGSLLGGKAALTLSGAQPEDEAEYYCALWYSNHWVFGGGTKLTVLI | |
| TCRαß (with | QNPDPAVYQLRDSKSSDKSVCLFTDFDSQTNVSQSKDSDVYITDKCVLDMRSMDFKSNSAVAWS | |
| SP) | NKSDFACANAFNNSIIPEDTFFPSPESSCDVKLVEKSFETDTNLNFQNLSVIGFRILLLKVAGFNLL | |
| [SP]-[VL1]- | MTLRLWSSGSGEGRGSLLTCGDVEENPGPMGWSCIILFLVATATGVHSDVLMTQTPLSLPVSLGD | |
| [TRAC_T47C]- | QASISCRSSQTIVHSEVQLVESGGGLVQPGGSLRLSCAASGFDFSRYWMNWVRQAPGKGLEWVG | |
| ][T2A]- | EITPDSSTINYAPSLKGRFTISRDNAKNSLYLQMNSLRAEDTAVYYCVRPYDYGAWFASWGQGT | |
| [SP]-[VH2]- | LVTVSSDLNKVFPPEVAVFEPSEAEISHTQKATLVCLATGFFPDHVELSWWVNGKEVHSGVCTDP | |
| [TRBC1_S56C] | QPLKEQPALNDSRYCLSSRLRVSATFWQNPRNHFRCQVQFYGLSENDEWTQDRAKPVTQIVSAE | |
| ] | AWGRADCGFTSVSYQQGVLSATILYEILLGKATLYAVLVSALVLMAMVKRKDF | |
| P329G | MGWSCIILFLVATATGVHSQAVVTQEPSLTVSPGGTVTLTCRSSTGAVTTSNYANWVQEKPDHLF | 272 |
| VH3VL1 | TGLIGGTNKRAPGTPARFSGSLLGGKAALTLSGAQPEDEAEYYCALWYSNHWVFGGGTKLTVLI | |
| TCRαß (with | QNPDPAVYQLRDSKSSDKSVCLFTDFDSQTNVSQSKDSDVYITDKCVLDMRSMDFKSNSAVAWS | |
| SP, T2A, | NKSDFACANAFNNSIIPEDTFFPSPESSCDVKLVEKSFETDTNLNFQNLSVIGFRILLLKVAGFNLL | |
| eGFP) | MTLRLWSSGSGEGRGSLLTCGDVEENPGPMGWSCIILFLVATATGVHSDVLMTQTPLSLPVSLGD | |
| [SP]-[VL1]- | QASISCRSSQTIVHSEVQLVESGGGLVQPGGSLRLSCAASGFTFSRYWMNWVRQAPGKGLEWVG | |
| [TRAC_T47C]- | EITPDSSTINYAPSLKGRFTISRDNAKNSLYLQMNSLRAEDTAVYYCARPYDYGAWFASWGQGT | |
| ][T2A]- | LVTVSSDLNKVFPPEVAVFEPSEAEISHTQKATLVCLATGFFPDHVELSWWVNGKEVHSGVCTDP | |
| [SP]-[VH3]- | QPLKEQPALNDSRYCLSSRLRVSATFWQNPRNHFRCQVQFYGLSENDEWTQDRAKPVTQIVSAE | |
| [TRBC1_S56C]- | AWGRADCGFTSVSYQQGVLSATILYEILLGKATLYAVLVSALVLMAMVKRKDFGSGEGRGSLLT | |
| ][T2A]- | CGDVEENPGPVSKGEELFTGVVPILVELDGDVNGHKFSVSGEGEGDATYGKLTLKFICTTGKLPV | |
| [eGFP] | PWPTLVTTLTYGVQCFSRYPDHMKQHDFFKSAMPEGYVQERTIFFKDDGNYKTRAEVKFEGDTL | |
| | VNRIELKGIDFKEDGNILGHKLEYNYNSHNVYIMADKQKNGIKVNFKIRHNIEDGSVQLADHYQQ | |
| | NTPIGDGPVLLPDNHYLSTQSALSKDPNEKRDHMVLLEFVTAAGITLGMDELYK | |
| P329G | MGWSCIILFLVATATGVHSQAVVTQEPSLTVSPGGTVTLTCRSSTGAVTTSNYANWVQEKPDHLF | 273 |
| VH3VL1 | TGLIGGTNKRAPGTPARFSGSLLGGKAALTLSGAQPEDEAEYYCALWYSNHWVFGGGTKLTVLI | |
| TCRαß (with | QNPDPAVYQLRDSKSSDKSVCLFTDFDSQTNVSQSKDSDVYITDKCVLDMRSMDFKSNSAVAWS | |
| SP) | NKSDFACANAFNNSIIPEDTFFPSPESSCDVKLVEKSFETDTNLNFQNLSVIGFRILLLKVAGFNLL | |
| [SP]-[VL1]- | MTLRLWSSGSGEGRGSLLTCGDVEENPGPMGWSCIILFLVATATGVHSDVLMTQTPLSLPVSLGD | |
| [TRAC_T47C]- | QASISCRSSQTIVHSEVQLVESGGGLVQPGGSLRLSCAASGFTFSRYWMNWVRQAPGKGLEWVG | |
| ][T2A]- | EITPDSSTINYAPSLKGRFTISRDNAKNSLYLQMNSLRAEDTAVYYCARPYDYGAWFASWGQGT | |
| [SP]-[VH3]- | LVTVSSDLNKVFPPEVAVFEPSEAEISHTQKATLVCLATGFFPDHVELSWWVNGKEVHSGVCTDP | |
| [TRBC1_S56C] | QPLKEQPALNDSRYCLSSRLRVSATFWQNPRNHFRCQVQFYGLSENDEWTQDRAKPVTQIVSAE | |
| | AWGRADCGFTSVSYQQGVLSATILYEILLGKATLYAVLVSALVLMAMVKRKDF | |
| P329G | MGWSCIILFLVATATGVHSQAVVTQEPSLTVSPGGTVTLTCRSSTGAVTTSNYANWVQEKPDHLF | 274 |
| VH1VL1 | TGLIGGTNKRAPGTPARFSGSLLGGKAALTLSGAQPEDEAEYYCALWYSNHWVFGGGTKLTVLD | |
| TCRαß (with | LNKVFPPEVAVFEPSEAEISHTQKATLVCLATGFFPDHVELSWWVNGKEVHSGVCTDPQPLKEQP | |
| SP, T2A, | ALNDSRYCLSSRLRVSATFWQNPRNHFRCQVQFYGLSENDEWTQDRAKPVTQIVSAEAWGRAD | |
| eGFP) | CGFTSVSYQQGVLSATILYEILLGKATLYAVLVSALVLMAMVKRKDFGSGEGRGSLLTCGDVEE | |
| [SP]-[VL1]- | NPGPMGWSCIILFLVATATGVHSDVLMTQTPLSLPVSLGDQASISCRSSQTIVHSEVQLVESGGGL | |
| [TRBC1_S56C]- | VQPGGSLRLSCAASGFDFSRYWMNWVRQAPGKGLEWVGEITPDSSTINYTPSLKGRFTISRDNAK | |
| ][T2A]- | NSLYLQMNSLRAEDTAVYYCVRPYDYGAWFASWGQGTLVTVSSIQNPDPAVYQLRDSKSSDKS | |
| [SP]-[VH1]- | VCLFTDFDSQTNVSQSKDSDVYITDKCVLDMRSMDFKSNSAVAWSNKSDFACANAFNNSIIPEDT | |
| [TRAC_T47C]- | FFPSPESSCDVKLVEKSFETDTNLNFQNLSVIGFRILLLKVAGFNLLMTLRLWSSGSGEGRGSLLTC | |
| ][T2A]- | GDVEENPGPVSKGEELFTGVVPILVELDGDVNGHKFSVSGEGEGDATYGKLTLKFICTTGKLPVP | |
| [eGFP] | WPTLVTTLTYGVQCFSRYPDHMKQHDFFKSAMPEGYVQERTIFFKDDGNYKTRAEVKFEGDTL | |
| | VNRIELKGIDFKEDGNILGHKLEYNYNSHNVYIMADKQKNGIKVNFKIRHNIEDGSVQLADHYQQ | |
| | NTPIGDGPVLLPDNHYLSTQSALSKDPNEKRDHMVLLEFVTAAGITLGMDELYK | |
| P329G | MGWSCIILFLVATATGVHSQAVVTQEPSLTVSPGGTVTLTCRSSTGAVTTSNYANWVQEKPDHLF | 275 |
| VH1VL1 | TGLIGGTNKRAPGTPARFSGSLLGGKAALTLSGAQPEDEAEYYCALWYSNHWVFGGGTKLTVLD | |
| TCRαß (with | LNKVFPPEVAVFEPSEAEISHTQKATLVCLATGFFPDHVELSWWVNGKEVHSGVCTDPQPLKEQP | |
| SP) | ALNDSRYCLSSRLRVSATFWQNPRNHFRCQVQFYGLSENDEWTQDRAKPVTQIVSAEAWGRAD | |
| [SP]-[VL1]- | CGFTSVSYQQGVLSATILYEILLGKATLYAVLVSALVLMAMVKRKDFGSGEGRGSLLTCGDVEE | |
| [TRBC1_S56C]- | NPGPMGWSCIILFLVATATGVHSDVLMTQTPLSLPVSLGDQASISCRSSQTIVHSEVQLVESGGGL | |
| ][T2A]- | VQPGGSLRLSCAASGFDFSRYWMNWVRQAPGKGLEWVGEITPDSSTINYTPSLKGRFTISRDNAK | |
| [SP]-[VH1]- | NSLYLQMNSLRAEDTAVYYCVRPYDYGAWFASWGQGTLVTVSSIQNPDPAVYQLRDSKSSDKS | |
| [TRAC_T47C] | VCLFTDFDSQTNVSQSKDSDVYITDKCVLDMRSMDFKSNSAVAWSNKSDFACANAFNNSIIPEDT | |
| ] | FFPSPESSCDVKLVEKSFETDTNLNFQNLSVIGFRILLLKVAGFNLLMTLRLWSS | |
| P329G | MGWSCIILFLVATATGVHSQAVVTQEPSLTVSPGGTVTLTCRSSTGAVTTSNYANWVQEKPDHLF | 276 |
| VH2VL1 | TGLIGGTNKRAPGTPARFSGSLLGGKAALTLSGAQPEDEAEYYCALWYSNHWVFGGGTKLTVLD | |
| TCRαß (with | LNKVFPPEVAVFEPSEAEISHTQKATLVCLATGFFPDHVELSWWVNGKEVHSGVCTDPQPLKEQP | |
| SP, T2A, | ALNDSRYCLSSRLRVSATFWQNPRNHFRCQVQFYGLSENDEWTQDRAKPVTQIVSAEAWGRAD | |
| eGFP) | CGFTSVSYQQGVLSATILYEILLGKATLYAVLVSALVLMAMVKRKDFGSGEGRGSLLTCGDVEE | |
| [SP]-[VL1]- | NPGPMGWSCIILFLVATATGVHSDVLMTQTPLSLPVSLGDQASISCRSSQTIVHSEVQLVESGGGL | |
| [TRBC1_S56C]- | VQPGGSLRLSCAASGFDFSRYWMNWVRQAPGKGLEWVGEITPDSSTINYAPSLKGRFTISRDNA | |
| ][T2A]- | KNSLYLQMNSLRAEDTAVYYCVRPYDYGAWFASWGQGTLVTVSSIQNPDPAVYQLRDSKSSDK | |
| [SP]-[VH2]- | SVCLFTDFDSQTNVSQSKDSDVYITDKCVLDMRSMDFKSNSAVAWSNKSDFACANAFNNSIIPED | |
| [TRAC_T47C]- | TFFPSPESSCDVKLVEKSFETDTNLNFQNLSVIGFRILLLKVAGFNLLMTLRLWSSGSGEGRGSLLT | |
| ][T2A]- | CGDVEENPGPVSKGEELFTGVVPILVELDGDVNGHKFSVSGEGEGDATYGKLTLKFICTTGKLPV | |
| [eGFP] | PWPTLVTTLTYGVQCFSRYPDHMKQHDFFKSAMPEGYVQERTIFFKDDGNYKTRAEVKFEGDTL | |
| | VNRIELKGIDFKEDGNILGHKLEYNYNSHNVYIMADKQKNGIKVNFKIRHNIEDGSVQLADHYQQ | |
| | NTPIGDGPVLLPDNHYLSTQSALSKDPNEKRDHMVLLEFVTAAGITLGMDELYK | |
| P329G | MGWSCIILFLVATATGVHSQAVVTQEPSLTVSPGGTVTLTCRSSTGAVTTSNYANWVQEKPDHLF | 277 |
| VH2VL1 | TGLIGGTNKRAPGTPARFSGSLLGGKAALTLSGAQPEDEAEYYCALWYSNHWVFGGGTKLTVLD | |
| TCRαß (with | LNKVFPPEVAVFEPSEAEISHTQKATLVCLATGFFPDHVELSWWVNGKEVHSGVCTDPQPLKEQP | |
| SP) | ALNDSRYCLSSRLRVSATFWQNPRNHFRCQVQFYGLSENDEWTQDRAKPVTQIVSAEAWGRAD | |
| [SP]-[VL1]- | CGFTSVSYQQGVLSATILYEILLGKATLYAVLVSALVLMAMVKRKDFGSGEGRGSLLTCGDVEE | |
| [TRBC1_S56C]- | NPGPMGWSCIILFLVATATGVHSDVLMTQTPLSLPVSLGDQASISCRSSQTIVHSEVQLVESGGGL | |
| ][T2A]- | VQPGGSLRLSCAASGFDFSRYWMNWVRQAPGKGLEWVGEITPDSSTINYAPSLKGRFTISRDNA | |
| [SP]-[VH2]- | KNSLYLQMNSLRAEDTAVYYCVRPYDYGAWFASWGQGTLVTVSSIQNPDPAVYQLRDSKSSDK | |
| [TRAC_T47C] | SVCLFTDFDSQTNVSQSKDSDVYITDKCVLDMRSMDFKSNSAVAWSNKSDFACANAFNNSIIPED | |
| ] | TFFPSPESSCDVKLVEKSFETDTNLNFQNLSVIGFRILLLKVAGFNLLMTLRLWSS | |
| P329G | MGWSCIILFLVATATGVHSQAVVTQEPSLTVSPGGTVTLTCRSSTGAVTTSNYANWVQEKPDHLF | 278 |
| VH3VL1 | TGLIGGTNKRAPGTPARFSGSLLGGKAALTLSGAQPEDEAEYYCALWYSNHWVFGGGTKLTVLD | |
| TCRαß (with | LNKVFPPEVAVFEPSEAEISHTQKATLVCLATGFFPDHVELSWWVNGKEVHSGVCTDPQPLKEQP | |
| SP, T2A, | ALNDSRYCLSSRLRVSATFWQNPRNHFRCQVQFYGLSENDEWTQDRAKPVTQIVSAEAWGRAD | |
| eGFP) | CGFTSVSYQQGVLSATILYEILLGKATLYAVLVSALVLMAMVKRKDFGSGEGRGSLLTCGDVEE | |
| [SP]-[VL1]- | NPGPMGWSCIILFLVATATGVHSDVLMTQTPLSLPVSLGDQASISCRSSQTIVHSEVQLVESGGGL | |
| [TRBC1_S56C]- | VQPGGSLRLSCAASGFTFSRYWMNWVRQAPGKGLEWVGEITPDSSTINYAPSLKGRFTISRDNAK | |
| ][T2A]- | NSLYLQMNSLRAEDTAVYYCARPYDYGAWFASWGQGTLVTVSSIQNPDPAVYQLRDSKSSDKS | |
| [SP]-[VH3]- | VCLFTDFDSQTNVSQSKDSDVYITDKCVLDMRSMDFKSNSAVAWSNKSDFACANAFNNSIIPEDT | |
| [TRAC_T47C]- | FFPSPESSCDVKLVEKSFETDTNLNFQNLSVIGFRILLLKVAGFNLLMTLRLWSSGSGEGRGSLLTC | |
| ][T2A]- | GDVEENPGPVSKGEELFTGVVPILVELDGDVNGHKFSVSGEGEGDATYGKLTLKFICTTGKLPVP | |
| [eGFP] | WPTLVTTLTYGVQCFSRYPDHMKQHDFFKSAMPEGYVQERTIFFKDDGNYKTRAEVKFEGDTL | |
| | VNRIELKGIDFKEDGNILGHKLEYNYNSHNVYIMADKQKNGIKVNFKIRHNIEDGSVQLADHYQQ | |
| | NTPIGDGPVLLPDNHYLSTQSALSKDPNEKRDHMVLLEFVTAAGITLGMDELYK | |
| P329G | MGWSCIILFLVATATGVHSQAVVTQEPSLTVSPGGTVTLTCRSSTGAVTTSNYANWVQEKPDHLF | 279 |
| VH3VL1 | TGLIGGTNKRAPGTPARFSGSLLGGKAALTLSGAQPEDEAEYYCALWYSNHWVFGGGTKLTVLD | |
| TCRαß (with | LNKVFPPEVAVFEPSEAEISHTQKATLVCLATGFFPDHVELSWWVNGKEVHSGVCTDPQPLKEQP | |
| SP) | ALNDSRYCLSSRLRVSATFWQNPRNHFRCQVQFYGLSENDEWTQDRAKPVTQIVSAEAWGRAD | |
| [SP]-[VL1]- | CGFTSVSYQQGVLSATILYEILLGKATLYAVLVSALVLMAMVKRKDFGSGEGRGSLLTCGDVEE | |
| [TRBC1_S56C]- | NPGPMGWSCIILFLVATATGVHSDVLMTQTPLSLPVSLGDQASISCRSSQTIVHSEVQLVESGGGL | |
| ][T2A]- | VQPGGSLRLSCAASGFTFSRYWMNWVRQAPGKGLEWVGEITPDSSTINYAPSLKGRFTISRDNAK | |
| [SP]-[VH3]- | NSLYLQMNSLRAEDTAVYYCARPYDYGAWFASWGQGTLVTVSSIQNPDPAVYQLRDSKSSDKS | |
| [TRAC_T47C] | VCLFTDFDSQTNVSQSKDSDVYITDKCVLDMRSMDFKSNSAVAWSNKSDFACANAFNNSIIPEDT | |
| ] | FFPSPESSCDVKLVEKSFETDTNLNFQNLSVIGFRILLLKVAGFNLLMTLRLWSS | |
| sgRNA | GGUGGCCACAAUUGUCAUAGGUUUUAGAGCUAGAAAUAGCAAGUUAAAAUAAGGCUAGU | 280 |
| targeting | CCGUUAUCAACUUGAAAAAGUGGCACCGAGUCGGUGCUUUU | |
| Exon 7 of | | |
| CD3E | | |
| sgRNA | GGAGAAUGACGAGUGGACCCGUUUUAGAGCUAGAAAUAGCAAGUUAAAAUAAGGCUAGU | 281 |
| targeting | CCGUUAUCAACUUGAAAAAGUGGCACCGAGUCGGUGCUUUU | |
| TRBC1 | | |
| sgRNA | AGAGUCUCUCAGCUGGUACAGUUUUAGAGCUAGAAAUAGCAAGUUAAAAUAAGGCUAGU | 282 |
| targeting | CCGUUAUCAACUUGAAAAAGUGGCACCGAGUCGGUGCUUUU | |
| TRAC | | |
| P2Acleavage | GSGATNFSLLKQAGDVEENPGP | 283 |
| site | | |
| F2Acleavage | GSGVKQTLNFDLLKLAGDVESNPGP | 284 |
| site | | |
| ITAM | YXXL/I | 285 |
| consensus | wherein X = any amino acid | |
| Larger ITAM | YXXL/I(X)₆₋₈YXXL/I | 286 |
| consensus | wherein X = any amino acid | |
| Nucleotide | GAAGTGCAGCTGGTTGAGTCTGGCGGAGGACTGGTTCAACCTGGCGGAAGCCTGAGACTGTC | 287 |
| encoding | TTGTGCCGCCAGCGGCTTCACCTTCAGCAGATACTGGATGAACTGGGTGAGACAGGCCCCTG | |
| P329G | GCAAAGGACTGGAGTGGGTGGGAGAGATCACCCCTGACAGCAGCACCATCAATTACGCCCC | |
| VH3VL1 | TAGCCTGAAGGGCAGATTCACCATCTCCAGAGACAACGCCAAGAACAGCCTGTACCTGCAGA | |
| CD38 | TGAACTCCCTGAGAGCCGAGGACACCGCCGTGTACTACTGTGCCAGACCTTACGATTACGGC | |
| (mature) | GCCTGGTTTGCTTCTTGGGGCCAGGGAACACTGGTGACAGTTAGCTCTGGAGGAGGAGGATC | |
| [VH3VL1 | AGGCGGAGGCGGAAGTGGTGGTGGAGGATCTCAGGCTGTGGTGACACAAGAGCCAAGCCTG | |
| scFv]-[CD38 | ACAGTGTCTCCAGGCGGCACAGTGACCCTGACCTGTAGATCTTCTACCGGAGCCGTGACCAC | |
| ECD]-[CD3ϵ | CAGCAACTACGCCAATTGGGTGCAAGAGAAGCCCGACCACCTGTTTACAGGCCTGATCGGCG | |
| TMD]-[CD3 | GCACCAATAAGAGAGCACCTGGCACACCAGCCAGATTCTCTGGATCTCTGCTCGGAGGAAAG | |
| ICD] | GCCGCTCTGACACTGTCTGGAGCCCAGCCAGAGGATGAGGCCGAGTATTATTGTGCCCTGTG | |
| | GTACAGCAACCACTGGGTGTTTGGCGGCGGAACAAAGCTGACAGTTCTTGGAGGTGGTGGAA | |
| | GCGGTGGAGGTGGATCTGGAGGTGGCGGATCCGATGGAAATGAGGAGATGGGCGGCATCAC | |
| | CCAGACACCTTACAAGGTGAGCATCAGCGGCACCACCGTGATCCTGACATGTCCTCAGTACC | |
| | CTGGCAGCGAGATCCTGTGGCAGCACAACGATAAGAACATCGGCGGCGACGAGGACGACAA | |
| | GAATATCGGCTCTGACGAGGATCACCTGTCTCTGAAGGAGTTCAGCGAGCTGGAGCAGAGCG | |
| | GCTACTACGTGTGTTACCCTAGAGGCAGCAAGCCCGAGGACGCCAACTTCTACCTGTATCTG | |
| | AGAGCCAGAGTGTGCGAGAACTGCATGGAGATGGATGTGATGAGTGTCGCTACCATCGTGAT | |
| | CGTCGACATCTGTATCACAGGCGGCCTGCTGCTGCTGGTGTACTATTGGAGCAAGAACCGGA | |
| | AGGCCAAAGCCAAGCCAGTGACAAGAGGAGCAGGAGCCGGAGGTAGACAGAGAGGCCAGA | |
| | ACAAAGAAAGGCCTCCACCGGTGCCTAATCCTGACTACGAGCCCATCAGAAAGGGCCAGAG | |
| | AGATCTGTACAGCGGCCTGAACCAGAGAAGAATC | |
| Nucleotide | GAGGTGCAGCTGGTGGAGAGCGGCGGCGGCCTGGTGCAGCCCGGCGGCAGCCTGAGGCTGA | 288 |
| encoding | GCTGCGCCGCCAGCGGCTTCACCTTCAGCAGGTACTGGATGAACTGGGTGAGGCAGGCCCCC | |
| P329G | GGCAAGGGCCTGGAGTGGGTGGGCGAGATCACCCCCGACAGCAGCACCATCAACTACGCCC | |
| TCRαß- | CCAGCCTGAAGGGCAGGTTCACCATCAGCAGGGACAACGCCAAGAACAGCCTGTACCTGCA | |
| VH3TRAC | GATGAACAGCCTGAGGGCCGAGGACACCGCCGTGTACTACTGCGCCAGGCCCTACGACTACG | |
| (mature) | GCGCCTGGTTCGCCAGCTGGGGCCAGGGCACCCTGGTGACCGTGAGCAGCATCCAGAACCCC | |
| [VH3]- | GACCCCGCAGTCTATCAATTGCGTGATAGTAAGAGTAGCGACAAGTCTGTGTGCCTGTTCAC | |
| [TRAC_T47C] | CGATTTCGACAGTCAGACAAATGTTTCTCAGAGTAAGGACAGTGATGTGTACATCACCGACA | |
| ] | AATGCGTCCTGGACATGCGCTCCATGGACTTCAAGAGTAATAGCGCCGTAGCCTGGAGCAAC | |
| | AAAAGCGACTTCGCATGTGCCAACGCGTTCAATAACTCCATTATCCCTGAAGATACTTTTTTC | |
| | CCCTCCCCCGAAAGTAGCTGCGATGTTAAACTCGTTGAGAAGTCCTTCGAAACTGACACCAA | |
| | CCTGAACTTCCAGAACCTGAGTGTGATTGGCTTCCGCATCCTGCTCCTGAAAGTGGCCGGCTT | |
| | TAACCTGCTCATGACACTGCGTCTGTGGTCAAGC | |
| Nucleotide | CAGGCCGTGGTGACCCAGGAGCCCAGCCTGACCGTGAGCCCCGGCGGCACCGTGACCCTGAC | 289 |
| encoding | CTGCAGGAGCAGCACCGGCGCCGTGACCACCAGCAACTACGCCAACTGGGTGCAGGAGAAG | |
| P329G | CCCGACCACCTGTTCACCGGCCTGATCGGCGGCACCAACAAGAGGGCCCCCGGCACCCCCGC | |
| TCRαß | CAGGTTCAGCGGCAGCCTGCTGGGCGGCAAGGCCGCCCTGACCCTGAGCGGCGCCCAGCCCG | |
| VLITRBC1 | AGGACGAGGCCGAGTACTACTGCGCCCTGTGGTACAGCAACCACTGGGTGTTCGGCGGCGGC | |
| (mature) | ACCAAGCTGACCGTCCTAGACCTGAATAAAGTGTTTCCCCCAGAGGTCGCCGTGTTTGAACC | |
| [VL1]- | GAGCGAGGCTGAGATCTCCCACACCCAGAAGGCTACACTGGTCTGTCTTGCCACCGGTTTCTT | |
| [TRBC1_S56C] | TCCTGACCACGTGGAACTGTCTTGGTGGGTTAACGGTAAGGAGGTGCACTCCGGTGTGTGCA | |
| ] | CTGATCCCCAGCCGTTGAAAGAACAGCCGGCTTTGAACGACTCCCGGTACTGCCTTAGCTCC | |
| | CGCCTGCGCGTGTCTGCTACATTCTGGCAGAACCCCCGCAACCACTTTCGGTGCCAGGTGCA | |
| | GTTTTACGGCCTGTCCGAGAACGATGAATGGACACAAGACCGGGCTAAGCCCGTGACTCAGA | |
| | TCGTGAGTGCTGAGGCTTGGGGGCGCGCCGATTGCGGCTTCACCTCAGTCAGCTACCAACAG | |
| | GGCGTTCTGAGTGCCACAATCCTGTATGAGATCCTCCTGGGAAAGGCAACTCTGTACGCTGTT | |
| | CTCGTCTCCGCGCTTGTTCTGATGGCTATGGTGAAGAGAAAGGATTTC | |
| Nucleotide | CAGGCCGTGGTGACCCAGGAGCCCAGCCTGACCGTGAGCCCCGGCGGCACCGTGACCCTGAC | 290 |
| encoding | CTGCAGGAGCAGCACCGGCGCCGTGACCACCAGCAACTACGCCAACTGGGTGCAGGAGAAG | |
| P329G | CCCGACCACCTGTTCACCGGCCTGATCGGCGGCACCAACAAGAGGGCCCCCGGCACCCCCGC | |
| TCRαß | CAGGTTCAGCGGCAGCCTGCTGGGCGGCAAGGCCGCCCTGACCCTGAGCGGCGCCCAGCCCG | |
| VLITRAC | AGGACGAGGCCGAGTACTACTGCGCCCTGTGGTACAGCAACCACTGGGTGTTCGGCGGCGGC | |
| (mature) | ACCAAGCTGACCGTCCTAATCCAGAACCCCGACCCCGCAGTCTATCAATTGCGTGATAGTAA | |
| [VL1]- | GAGTAGCGACAAGTCTGTGTGCCTGTTCACCGATTTCGACAGTCAGACAAATGTTTCTCAGA | |
| [TRAC_T47C] | GTAAGGACAGTGATGTGTACATCACCGACAAATGCGTCCTGGACATGCGCTCCATGGACTTC | |
| ] | AAGAGTAATAGCGCCGTAGCCTGGAGCAACAAAAGCGACTTCGCATGTGCCAACGCGTTCA | |
| | ATAACTCCATTATCCCTGAAGATACTTTTTTCCCCTCCCCCGAAAGTAGCTGCGATGTTAAAC | |
| | TCGTTGAGAAGTCCTTCGAAACTGACACCAACCTGAACTTCCAGAACCTGAGTGTGATTGGC | |
| | TTCCGCATCCTGCTCCTGAAAGTGGCCGGCTTTAACCTGCTCATGACACTGCGTCTGTGGTCA | |
| | AGC | |
| Nucleotide | GAGGTGCAGCTGGTGGAGAGCGGCGGCGGCCTGGTGCAGCCCGGCGGCAGCCTGAGGCTGA | 291 |
| encoding | GCTGCGCCGCCAGCGGCTTCACCTTCAGCAGGTACTGGATGAACTGGGTGAGGCAGGCCCCC | |
| P329G | GGCAAGGGCCTGGAGTGGGTGGGCGAGATCACCCCCGACAGCAGCACCATCAACTACGCCC | |
| TCRαß- | CCAGCCTGAAGGGCAGGTTCACCATCAGCAGGGACAACGCCAAGAACAGCCTGTACCTGCA | |
| VH3TRBC1 | GATGAACAGCCTGAGGGCCGAGGACACCGCCGTGTACTACTGCGCCAGGCCCTACGACTACG | |
| (mature) | GCGCCTGGTTCGCCAGCTGGGGCCAGGGCACCCTGGTGACCGTGAGCAGCGACCTGAATAAA | |
| [VH3]- | GTGTTTCCCCCAGAGGTCGCCGTGTTTGAACCGAGCGAGGCTGAGATCTCCCACACCCAGAA | |
| [TRBC1_S56C] | GGCTACACTGGTCTGTCTTGCCACCGGTTTCTTTCCTGACCACGTGGAACTGTCTTGGTGGGT | |
| ] | TAACGGTAAGGAGGTGCACTCCGGTGTGTGCACTGATCCCCAGCCGTTGAAAGAACAGCCGG | |
| | CTTTGAACGACTCCCGGTACTGCCTTAGCTCCCGCCTGCGCGTGTCTGCTACATTCTGGCAGA | |
| | ACCCCCGCAACCACTTTCGGTGCCAGGTGCAGTTTTACGGCCTGTCCGAGAACGATGAATGG | |
| | ACACAAGACCGGGCTAAGCCCGTGACTCAGATCGTGAGTGCTGAGGCTTGGGGGCGCGCCG | |
| | ATTGCGGCTTCACCTCAGTCAGCTACCAACAGGGCGTTCTGAGTGCCACAATCCTGTATGAG | |
| | ATCCTCCTGGGAAAGGCAACTCTGTACGCTGTTCTCGTCTCCGCGCTTGTTCTGATGGCTATG | |
| | GTGAAGAGAAAGGATTTC | |
| αFolR1 | NAWMS | 292 |
| HCDR1 | | |
| αFolR1 | RIKSKTDGGTTDYAAPVKG | 293 |
| HCDR2 | | |
| αFolR1 | PWEWSWYDY | 294 |
| HCDR3 | | |
| αFolR1 | GSSTGAVTTSNYAN | 295 |
| LCDR1 | | |
| αFolR1 | GTNKRAP | 296 |
| LCDR2 | | |
| αFolR1 | ALWYSNLWV | 297 |
| LCDR3 | | |
| αFolR1 | EVQLVESGGGLVKPGGSLRLSCAASGFTFSNAWMSWVRQAPGKGLEWVGRIKSKTDGGTTDYA | 298 |
| VH | APVKGRFTISRDDSKNTLYLQMNSLKTEDTAVYYCTTPWEWSWYDYWGQGTLVTVSS | |
| αFolR1 | QAVVTQEPSLTVSPGGTVTLTCGSSTGAVTTSNYANWVQEKPGQAFRGLIGGTNKRAPGTPARFS | 299 |
| VL | GSLLGGKAALTLSGAQPEDEAEYYCALWYSNLWVFGGGTKLTVL | |
| αFolR1 | EVQLVESGGGLVKPGGSLRLSCAASGFTFSNAWMSWVRQAPGKGLEWVGRIKSKTDGGTTDYA | 300 |
| HC | APVKGRFTISRDDSKNTLYLQMNSLKTEDTAVYYCTTPWEWSWYDYWGQGTLVTVSSASTKGP | |
| L234A | SVFPLAPSSKSTSGGTAALGCLVKDYFPEPVTVSWNSGALTSGVHTFPAVLQSSGLYSLSSVVTVP | |
| L235A | SSSLGTQTYICNVNHKPSNTKVDKKVEPKSCDKTHTCPPCPAPEAAGGPSVFLFPPKPKDTLMISR | |
| P329G | TPEVTCVVVDVSHEDPEVKFNWYVDGVEVHNAKTKPREEQYNSTYRVVSVLTVLHQDWLNGK | |
| | EYKCKVSNKALGAPIEKTISKAKGQPREPQVYTLPPSRDELTKNQVSLTCLVKGFYPSDIAVEWES | |
| | NGQPENNYKTTPPVLDSDGSFFLYSKLTVDKSRWQQGNVFSCSVMHEALHNHYTQKSLSLSPGK | |
| αFolR1 | QAVVTQEPSLTVSPGGTVTLTCGSSTGAVTTSNYANWVQEKPGQAFRGLIGGTNKRAPGTPARFS | 301 |
| LC | GSLLGGKAALTLSGAQPEDEAEYYCALWYSNLWVFGGGTKLTVLGQPKAAPSVTLFPPSSEELQ | |
| | ANKATLVCLISDFYPGAVTVAWKADSSPVKAGVETTTPSKQSNNKYAASSYLSLTPEQWKSHRS | |
| | YSCQVTHEGSTVEKTVAPTECS | |
| αCEACAM5 | DTYMH | 302 |
| HCDR1 | | |
| αCEACAM5 | RIDPANGNSKYVPKFQG | 303 |
| HCDR2 | | |
| αCEACAM5 | FGYYVSDYAMAY | 304 |
| HCDR3 | | |
| αCEACAM5 | RAGESVDIFGVGFLH | 305 |
| LCDR1 | | |
| αCEACAM5 | RASNLES | 306 |
| LCDR2 | | |
| αCEACAM5 | QQTNEDPYT | 307 |
| LCDR3 | | |
| αCEACAM5 | EVQLQQSGAELVEPGASVKLSCTASGFNIKDTYMHWVKQRPEQGLEWIGRIDPANGNSKYVPKF | 308 |
| VH | QGKATITADTSSNTAYLQLTSLTSEDTAVYYCAPFGYYVSDYAMAYWGQGTSVTVSS | |
| αCEACAM5 | DIVLTQSPASLAVSLGQRATMSCRAGESVDIFGVGFLHWYQQKPGQPPKLLIYRASNLESGIPVRF | 309 |
| VL | SGTGSRTDFTLIIDPVEADDVATYYCQQTNEDPYTFGGGTKLEIK | |
| αCEACAM5 | EVQLQQSGAELVEPGASVKLSCTASGFNIKDTYMHWVKQRPEQGLEWIGRIDPANGNSKYVPKF | 310 |
| HC | QGKATITADTSSNTAYLQLTSLTSEDTAVYYCAPFGYYVSDYAMAYWGQGTSVTVSSASTKGPS | |
| | VFPLAPSSKSTSGGTAALGCLVKDYFPEPVTVSWNSGALTSGVHTFPAVLQSSGLYSLSSVVTVPS | |
| | SSLGTQTYICNVNHKPSNTKVDKKVEPKSCDKTHTCPPCPAPEAAGGPSVFLFPPKPKDTLMISRT | |
| | PEVTCVVVDVSHEDPEVKFNWYVDGVEVHNAKTKPREEQYNSTYRVVSVLTVLHQDWLNGKE | |
| | YKCKVSNKALGAPIEKTISKAKGQPREPQVYTLPPSRDELTKNQVSLTCLVKGFYPSDIAVEWES | |
| | NGQPENNYKTTPPVLDSDGSFFLYSKLTVDKSRWQQGNVFSCSVMHEALHNHYTQKSLSLSPG | |
| αCEACAM5 | DIVLTQSPASLAVSLGQRATMSCRAGESVDIFGVGFLHWYQQKPGQPPKLLIYRASNLESGIPVRF | 311 |
| LC | SGTGSRTDFTLIIDPVEADDVATYYCQQTNEDPYTFGGGTKLEIKRTVAAPSVFIFPPSDEQLKSGT | |
| L234A | ASVVCLLNNFYPREAKVQWKVDNALQSGNSQESVTEQDSKDSTYSLSSTLTLSKADYEKHKVY | |
| L235A | ACEVTHQGLSSPVTKSFNRGEC | |
| P329G | | |
| αDP47 | SYAMS | 312 |
| HCDR1 | | |
| αDP47 | AISGSGGSTYYADSVKG | 313 |
| HCDR2 | | |
| αDP47 | GSGFDY | 314 |
| HCDR3 | | |
| αDP47 | RASQSVSSSYLA | 315 |
| LCDR1 | | |
| αDP47 | GASSRAT | 316 |
| LCDR2 | | |
| αDP47 | QQYGSSPLT | 317 |
| LCDR3 | | |
| αDP47 | EVQLLESGGGLVQPGGSLRLSCAASGFTFSSYAMSWVRQAPGKGLEWVSAISGSGGSTYYADSV | 318 |
| VH | KGRFTISRDNSKNTLYLQMNSLRAEDTAVYYCAKGSGFDYWGQGTLVTVSS | |
| αDP47 | EIVLTQSPGTLSLSPGERATLSCRASQSVSSSYLAWYQQKPGQAPRLLIYGASSRATGIPDRFSGSG | 319 |
| VL | SGTDFTLTISRLEPEDFAVYYCQQYGSSPLTFGQGTKVEIK | |
| αDP47 | EVQLLESGGGLVQPGGSLRLSCAASGFTFSSYAMSWVRQAPGKGLEWVSAISGSGGSTYYADSV | 320 |
| HC | KGRFTISRDNSKNTLYLQMNSLRAEDTAVYYCAKGSGFDYWGQGTLVTVSSASTKGPSVFPLAP | |
| L234A | SSKSTSGGTAALGCLVKDYFPEPVTVSWNSGALTSGVHTFPAVLQSSGLYSLSSVVTVPSSSLGTQ | |
| L235A | TYICNVNHKPSNTKVDKKVEPKSCDKTHTCPPCPAPEAAGGPSVFLFPPKPKDTLMISRTPEVTCV | |
| P329G | VVDVSHEDPEVKFNWYVDGVEVHNAKTKPREEQYNSTYRVVSVLTVLHQDWLNGKEYKCKVS | |
| | NKALGAPIEKTISKAKGQPREPQVYTLPPSRDELTKNQVSLTCLVKGFYPSDIAVEWESNGQPENN | |
| | YKTTPPVLDSDGSFFLYSKLTVDKSRWQQGNVFSCSVMHEALHNHYTQKSLSLSPGK | |
| αDP47 | EIVLTQSPGTLSLSPGERATLSCRASQSVSSSYLAWYQQKPGQAPRLLIYGASSRATGIPDRFSGSG | 321 |
| LC | SGTDFTLTISRLEPEDFAVYYCQQYGSSPLTFGQGTKVEIKRTVAAPSVFIFPPSDEQLKSGTASVV | |
| | CLLNNFYPREAKVQWKVDNALQSGNSQESVTEQDSKDSTYSLSSTLTLSKADYEKHKVYACEVT | |
| | HQGLSSPVTKSFNRGEC | |
| PD1-reg-IL2v | APASSSTKKTQLQLEHLLLDLQMILNGINNYKNPKLTRMLTAKFAMPKKATELKHLQCLEEELKP | 322 |
| IL2-(linker)- | LEEVLNGAQSKNFHLRPRDLISNINVIVLELKGSETTFMCEYADETATIVEFLNRWITFAQSIISTLT | |
| heavy chain | GGSGGGGSGGGGSGGGGSGGQVQLVQSGAEVKKPGASVKVSCKASGDTFTRYYVHWVRQAPG | |
| | QGLEWMGIINPSGGYASYAQKFQGRVTMTRDTSTSTVYMELSSLRSEDTAVYYCAAGLFIWGQG | |
| | TLVTVSSASVAAPSVFIFPPSDEQLKSGTASVVCLLNNFYPREAKVQWKVDNALQSGNSQESVTE | |
| | QDSKDSTYSLSSTLTLSKADYEKHKVYACEVTHQGLSSPVTKSFNRGECDKTHTCPPCPAPEAAG | |
| | GPSVFLFPPKPKDTLMISRTPEVTCVVVDVSHEDPEVKFNWYVDGVEVHNAKTKPREEQYNSTY | |
| | RVVSVLTVLHQDWLNGKEYKCKVSNKALGAPIEKTISKAKGQPREPQVYTLPPCRDELTKNQVS | |
| | LWCLVKGFYPSDIAVEWESNGQPENNYKTTPPVLDSDGSFFLYSKLTVDKSRWQQGNVFSCSVM | |
| | HEALHNHYTQKSLSLSPG | |
| PD1-reg-IL2v | DIQMTQSPSSLSASVGDRVTITCRASQSIGRYLAWYQQKPGKAPKLLIYSASNLETGVPSRFSGSG | 323 |
| Light chain | SGTDFTLTISSLQPEDFATYYCQQYNSFPVTFGPGTKVDIKSSASTKGPSVFPLAPSSKSTSGGTAA | |
| | LGCLVKDYFPEPVTVSWNSGALTSGVHTFPAVLQSSGLYSLSSVVTVPSSSLGTQTYICNVNHKPS | |
| | NTKVDKKVEPKSCD | |
| PD1-reg-IL2c | EVQLLESGGGLVQPGGSLRLSCAASGFSFSSYTMSWVRQAPGKGLEWVSAISGGGRDIYYPDSV | 324 |
| Anti-PD1 | KGRFTISRDNSKNTLYLQMNSLRAEDTAVYYCVLLTGRVYFALDSWGQGTLVTVSSASTKGPSV | |
| heavy chain | FPLAPSSKSTSGGTAALGCLVKDYFPEPVTVSWNSGALTSGVHTFPAVLQSSGLYSLSSVVTVPSS | |
| | SLGTQTYICNVNHKPSNTKVDKKVEPKSCDKTHTCPPCPAPEAAGGPSVFLFPPKPKDTLMISRTP | |
| | EVTCVVVDVSHEDPEVKFNWYVDGVEVHNAKTKPREEQYNSTYRVVSVLTVLHQDWLNGKEY | |
| | KCKVSNKALGAPIEKTISKAKGQPREPQVCTLPPSRDELTKNQVSLSCAVKGFYPSDIAVEWESN | |
| | GQPENNYKTTPPVLDSDGSFFLVSKLTVDKSRWQQGNVFSCSVMHEALHNHYTQKSLSLSPG | |
| PD1-reg-IL2c | DIVMTQSPDSLAVSLGERATINCKASESVDTSDNSFIHWYQQKPGQSPKLLIYRASTLESGVPDRF | 325 |
| Anti-PD1 | SGSGSGTDFTLTISSLQAEDVAVYYCQQNYDVPWTFGQGTKVEIKRTVAAPSVFIFPPSDEQLKSG | |
| light chain | TASVVCLLNNFYPREAKVQWKVDNALQSGNSQESVTEQDSKDSTYSLSSTLTLSKADYEKHKVY | |
| OA-PD1-reg- | ACEVTHQGLSSPVTKSFNRGEC | |
| IL2v | DKTHTCPPCPAPEAAGGPSVFLFPPKPKDTLMISRTPEVTCVVVDVSHEDPEVKFNWYVDGVEVH | 326 |
| Heavy chain | NAKTKPREEQYNSTYRVVSVLTVLHQDWLNGKEYKCKVSNKALGAPIEKTISKAKGQPREPQVC | |
| | TLPPSRDELTKNQVSLSCAVKGFYPSDIAVEWESNGQPENNYKTTPPVLDSDGSFFLVSKLTVDKS | |
| | RWQQGNVFSCSVMHEALHNHYTQKSLSLSPG | |
| OA-PD1-reg- | DIQMTQSPSSLSASVGDRVTITCRASQSIGRYLAWYQQKPGKAPKLLIYSASNLETGVPSRFSGSG | 327 |
| IL2v | SGTDFTLTISSLQPEDFATYYCQQYNSFPVTFGPGTKVDIKRTVAAPSVFIFPPSDEQLKSGTASVV | |
| Light chain | CLLNNFYPREAKVQWKVDNALQSGNSQESVTEQDSKDSTYSLSSTLTLSKADYEKHKVYACEVT | |
| | HQGLSSPVTKSFNRGEC | |
| OA-PD1-reg- | APASSSTKKTQLQLEHLLLDLQMILNGINNYKNPKLTRMLTAKFAMPKKATELKHLQCLEEELKP | 328 |
| IL2v | LEEVLNGAQSKNFHLRPRDLISNINVIVLELKGSETTFMCEYADETATIVEFLNRWITFAQSIISTLT | |
| IL2-(linker)- | GGSGGGGSGGGGSGGGGSGGQVQLVQSGAEVKKPGASVKVSCKASGDTFTRYYVHWVRQAPG | |
| heavy chain | QGLEWMGIINPSGGYASYAQKFQGRVTMTRDTSTSTVYMELSSLRSEDTAVYYCAAGLFIWGQG | |
| | TLVTVSSASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVL | |
| | TVLHQDWLNGKEYKCKVSNKALGAPIEKTISKAKGQPREPQVYTLPPCRDELTKNQVSLWCLVK | |
| | GFYPSDIAVEWESNGQPENNYKTTPPVLDSDGSFFLYSKLTVDKSRWQQGNVFSCSVMHEALHN | |
| | HYTQKSLSLSPG | |

TABLE A

| | Column A | | | Column B | | |
|---|---|---|---|---|---|---|
| | VH | | | VL | | |
| Antibody | HC-CDR1 | HC-CDR2 | HC-CDR3 | LC-CDR1 | LC-CDR2 | LC-CDR3 |
| αP329G_VH1/VL1 | SEQ ID NO: 11 | SEQ ID NO: 12 | SEQ ID NO: 13 | SEQ ID NO: 24 | SEQ ID NO: 25 | SEQ ID NO: 26 |
| αP329G_VH2/VL1 | SEQ ID NO: 11 | SEQ ID NO: 19 | SEQ ID NO: 13 | SEQ ID NO: 24 | SEQ ID NO: 25 | SEQ ID NO: 26 |
| αP329G_VH3/VL1 | SEQ ID NO: 11 | SEQ ID NO: 19 | SEQ ID NO: 13 | SEQ ID NO: 24 | SEQ ID NO: 25 | SEQ ID NO: 26 |

TABLE B

| | Column A | | | | Column B | | | |
|---|---|---|---|---|---|---|---|---|
| | VH | | | | VL | | | |
| Antibody | HC-FR1 | HC-FR2 | HC-FR3 | HC-FR4 | LC-FR1 | LC-FR2 | LC-FR3 | LC-FR4 |
| αP329G_VH1/VL1 | SEQ ID | SEQ ID | SEQ ID | SEQ ID | SEQ ID | SEQ ID | SEQ ID | SEQ ID |
| | NO: 14 | NO: 15 | NO: 16 | NO: 17 | NO: 27 | NO: 28 | NO: 29 | NO: 30 |
| αP329G_VH2/VL1 | SEQ ID | SEQ ID | SEQ ID | SEQ ID | SEQ ID | SEQ ID | SEQ ID | SEQ ID |
| | NO: 14 | NO: 15 | NO: 16 | NO: 17 | NO: 27 | NO: 28 | NO: 29 | NO: 30 |
| αP329G_VH3/VL1 | SEQ ID | SEQ ID | SEQ ID | SEQ ID | SEQ ID | SEQ ID | SEQ ID | SEQ ID |
| | NO: 21 | NO: 15 | NO: 22 | NO: 17 | NO: 27 | NO: 28 | NO: 29 | NO: 30 |

TABLE C

| | Column A | Column B |
|---|---|---|
| Antibody | VH | VL |
| αP329G_VH1/VL1 | SEQ ID NO: 10 | SEQ ID NO: 23 |
| αP329G_VH2/VL1 | SEQ ID NO: 18 | SEQ ID NO: 23 |
| αP329G_VH3/VL1 | SEQ ID NO: 20 | SEQ ID NO: 23 |

The present disclosure includes the combination of the aspects and preferred features described except where such a combination is clearly impermissible or expressly avoided.

The section headings used herein are for organisational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, a “peptide” refers to a chain of two or more amino acid monomers linked by peptide bonds. A peptide typically has a length in the region of about 2 to 50 amino acids. A “polypeptide” is a polymer chain of two or more peptides. Polypeptides typically have a length greater than about 50 amino acids.

As used herein, an amino acid sequence, or a region of a polypeptide which “corresponds” to a specified reference amino acid sequence or region of a polypeptide has at least 60%, e.g. one of at least ≥65%, ≥70%, ≥75%, ≥80%, ≥85%, ≥90%, ≥91%, ≥92%, ≥93%, ≥94%, ≥95%, ≥96%, ≥97%, ≥98%, ≥99% or 100% sequence identity to the amino acid sequence of the amino acid sequence/polypeptide/region. An amino acid sequence/region/position of a polypeptide/amino acid sequence which “corresponds” to a specified reference amino acid sequence/region/position of a polypeptide/amino acid sequence can be identified by sequence alignment of the subject sequence to the reference sequence, e.g. using sequence alignment software such as ClustalOmega (Söding. J. 2005. Bioinformatics 21, 951-960).

It must be noted that, as used in the specification and the appended claims, the singular forms “a”, “an”, and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about”, it will be understood that the particular value forms another embodiment.

Where a nucleic acid sequence is disclosed herein, the reverse complement thereof is also expressly contemplated.

Methods described herein may preferably be performed in vitro. The term “in vitro” is intended to encompass procedures performed with cells in culture whereas the term “in vivo” is intended to encompass procedures with/on intact multi-cellular organisms.

As referred to herein, a “recombinant” polypeptide refers to a non-naturally-occurring polypeptide. A recombinant polypeptide may also be referred to as a “synthetic” polypeptide. A recombinant polypeptide may comprise or consist of an amino acid sequence that is not encoded by the genome of a naturally-occurring organism (e.g. a wildtype organism). That is, a recombinant polypeptide may comprise or consist of an amino acid sequence which is not comprised in the amino acid sequence of a polypeptide produced by a naturally-occurring organism. A recombinant polypeptide may be encoded by nucleic acid produced using recombinant nucleic acid techniques. A recombinant polypeptide may be produced by expression (e.g. by transcription, translation and any subsequent post-translational processing) from recombinant nucleic acid encoding the polypeptide. Recombinant nucleic acid techniques include techniques for constructing and manipulating the nucleotide sequences of nucleic acids, and include molecular cloning.

As used herein, the term “antigen binding molecule” refers in its broadest sense to a molecule that specifically binds an antigenic determinant. Examples of antigen binding molecules are antibodies, antibody fragments and scaffold antigen binding proteins.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “antigen binding moiety”, “antigen binding domain” or “antigen-binding portion of an antibody” when used herein refers to the part of an antibody that comprises the area which specifically binds to and is complementary to part or all of an antigen. The term thus refers to the amino acid residues of an antibody which are responsible for antigen-binding. An antigen binding domain may be provided by, for example, one or more antibody variable domains (also called antibody variable regions). Particularly, an antigen binding domain comprises an antibody light chain variable region (VL) and an antibody heavy chain variable region (VH). The antigen-binding portion of an antibody comprises amino acid residues from the “complementary determining regions” or “CDRs”, “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially. CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service. National Institutes of Health, Bethesda, MD (1991) and/or those residues from a “hypervariable loop”. The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “epitope” denotes a protein determinant of an antigen, such as a CEA or FolR1, capable of specifically binding to an antibody. Epitopes usually consist of chemically active surface groupings of molecules such as amino acids or sugar side chains and usually epitopes have specific three dimensional structural characteristics, as well as specific charge characteristics. Conformational and nonconformational epitopes are distinguished in that the binding to the former but not the latter is lost in the presence of denaturing solvents.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Amino acid sequences of heavy chains including Fc domains (or a subunit of an Fc domain as defined herein) are denoted herein without C-terminal glycine-lysine dipeptide if not indicated otherwise. In one embodiment of the invention, a heavy chain including a subunit of an Fc domain as specified herein, comprises an additional C-terminal glycine-lysine dipeptide (G446 and K447, numbering according to EU index of Kabat). In one embodiment of the invention, a heavy chain including a subunit of an Fc domain as specified herein, comprises an additional C-terminal glycine residue (G446, numbering according to EU index of Kabat). Compositions of the invention, such as the pharmaceutical compositions described herein, comprise a population of antigen binding molecules of the invention. The population of antigen binding molecule may comprise molecules having a full-length heavy chain and molecules having a cleaved variant heavy chain. The population of antigen binding molecules may consist of a mixture of molecules having a full-length heavy chain and molecules having a cleaved variant heavy chain, wherein at least 50%, at least 60%, at least 70%, at least 80% or at least 90% of the antigen binding molecules have a cleaved variant heavy chain. In one embodiment of the invention a composition comprising a population of antigen binding molecules of the invention comprises an antigen binding molecule comprising a heavy chain including a subunit of an Fc domain as specified herein with an additional C-terminal glycine-lysine dipeptide (G446 and K447, numbering according to EU index of Kabat). In one embodiment of the invention a composition comprising a population of antigen binding molecules of the invention comprises an immune activating Fc domain binding molecule comprising a heavy chain including a subunit of an Fc domain as specified herein with an additional C-terminal glycine residue (G446, numbering according to EU index of Kabat). In one embodiment of the invention such a composition comprises a population of antigen binding molecules comprised of molecules comprising a heavy chain including a subunit of an Fc domain as specified herein; molecules comprising a heavy chain including a subunit of a Fc domain as specified herein with an additional C-terminal glycine residue (G446, numbering according to EU index of Kabat); and molecules comprising a heavy chain including a subunit of an Fc domain as specified herein with an additional C-terminal glycine-lysine dipeptide (G446 and K447, numbering according to EU index of Kabat). Unless otherwise specified herein, numbering of amino acid residues in the Fc region or constant region is according to the EU numbering system, also called the EU index, as described in Kabat et al., Sequences of Proteins of Immunological Interest. 5th Ed. Public Health Service. National Institutes of Health, Bethesda, MD. 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

As used herein, the term “Fc domain-IL2 variant polypeptide” or “Fc-IL2v polypeptide” refers to a polypeptide molecule that includes at least one variant CH2-CH3 region and at least one IL-2 variant polypeptide. The variant CH2-CH3 region can be joined to the IL-2 polypeptide by a variety of interactions and in a variety of configurations as described herein. In particular embodiments, the variant CH2-CH3 region is fused to the IL-2 polypeptide via a peptide linker. Particular Fc-IL2v polypeptides according to the invention essentially consist of one variant CH2-CH3 region and one IL-2 variant polypeptide joined by one or more linker sequences.

By “fused” is meant that the components (e.g. variant CH2-CH3 region and an IL-2 variant polypeptide) are linked by peptide bonds, either directly or via one or more peptide linkers.

As used herein, the terms “first” and “second”, e.g. with respect to first and second polypeptide of an Fc-IL2v polypeptide complex etc., are used for convenience of distinguishing when there is more than one of each type of polypeptide. Use of these terms is not intended to confer a specific order or orientation unless explicitly so stated.

In one embodiment a recombinant Fc-IL2v polypeptide complex or an antibody described herein comprises an Fc domain derived from human origin and preferably all other parts of the human constant regions. As used herein the term “Fc domain derived from human origin” denotes a Fc domain which is either a Fc domain of a human antibody of the subclass IgG₁, IgG₂, IgG₃ or IgG₄, preferably a Fc domain from human IgG₁ subclass, a mutated Fc domain from human IgG1 subclass (in one embodiment with a mutation on L234A+L235A), a Fc domain from human IgG₄ subclass or a mutated Fc domain from human IgG₄ subclass (in one embodiment with a mutation on S228P). In one embodiment said antibodies have reduced or minimal effector function. In one embodiment the minimal effector function results from an effectorless Fc mutation. In one embodiment the effectorless Fc mutation is L234A/L235A or L234A/L235A/P329G or N297A or D265A/N297A. In one embodiment the effectorless Fc mutation is selected for each of the antibodies independently of each other from the group comprising (consisting of) L234A/L235A, L234A/L235A/P329G, N297A and D265A/N297A (EU numbering).

In one embodiment the recombinant Fc-IL2v polypeptide complex or antibodies described herein are of human IgG class (i.e. of IgG₁, IgG₂, IgG₃ or IgG₄ subclass).

In a preferred embodiment the recombinant Fc-IL2v polypeptide complex or antibodies described herein are of human IgG₁ subclass or of human IgG₄ subclass. In one embodiment the recombinant Fc-IL2v polypeptide complex or antibodies described herein are of human IgG, subclass. In one embodiment the recombinant Fc-IL2v polypeptide complex or antibodies described herein are of human IgG₄ subclass.

In one embodiment a recombinant Fc-IL2v polypeptide complex or an antibody described herein is characterized in that the constant chains are of human origin. Such constant chains are well known in the state of the art and e.g. described by Kabat, E. A., (see e.g. Johnson, G, and Wu, T. T., Nucleic Acids Res. 28 (2000) 214-218).

The terms “nucleic acid” or “nucleic acid molecule”, as used herein, are intended to include DNA molecules and RNA molecules. A nucleic acid molecule may be single-stranded or double-stranded, but preferably is double-stranded DNA.

The term “amino acid” as used within this application denotes the group of naturally occurring carboxy alpha-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program. By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

A method of producing a recombinant Fc-IL2v polypeptide complex or antibody described herein is provided, wherein the method comprises culturing a host cell comprising a polynucleotide encoding the recombinant Fc-IL2v polypeptide complex or antibody, as provided herein, under conditions suitable for expression of the recombinant Fc-IL2v polypeptide complex or antibody, and recovering the immunoconjugate or bispecific antibody from the host cell (or host cell culture medium).

The components of the recombinant Fc-IL2v polypeptide complex or antibody are genetically fused to each other. A recombinant Fc-IL2v polypeptide complexes or antibody can be designed such that its components are fused directly to each other or indirectly through a linker sequence. The composition and length of the linker may be determined in accordance with methods well known in the art and may be tested for efficacy. Additional sequences may also be included to incorporate a cleavage site to separate the individual components of the fusion if desired, for example an endopeptidase recognition sequence.

Antigen binding moieties comprise at least an antibody variable region capable of binding an antigenic determinant. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory. 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty). Antigen binding moieties and methods for producing the same are also described in detail in PCT publication WO 2011/020783, the entire content of which is incorporated herein by reference.

Any animal species of antibody, antibody fragment, antigen binding domain or variable region can be used in the recombinant Fc-IL2v polypeptide complexes or antibody described herein. Non-limiting antibodies, antibody fragments, antigen binding domains or variable regions useful in the present invention can be of murine, primate, or human origin. Where the recombinant Fc-IL2v polypeptide complexes or antibody is intended for human use, a chimeric form may be used wherein the constant regions of the antibody are from a human. A humanized or fully human form of the antibody can also be prepared in accordance with methods well known in the art (see e.g. U.S. Pat. No. 5,565,332). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs: the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988): Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeven et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260) (2000) (describing the “guided selection” approach to FR shuffling). Human antibodies and human variable regions can be produced using various techniques known in the art. Human antibodies are described generally in van Dijk and van de Winkel. Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al, in Methods in Molecular Biology 178, 1-37 (O'Brien et al., ed., Human Press, Totowa, NJ. 2001); and McCafferty et al., Nature 348, 552-554; Clackson et al., Nature 352, 624-628 (1991)). Phage typically display antibody fragments, either as single-chain Fv (scFv) fragments or as Fab fragments. A detailed description of the preparation of antigen binding moieties by phage display can be found in the Examples appended to PCT publication WO 2011/020783.

In certain embodiments, antibodies are engineered to have enhanced binding affinity according to, for example, the methods disclosed in PCT publication WO 2011/020783 (see Examples relating to affinity maturation) or U.S. Pat. Appl. Publ. No. 2004/0132066, the entire contents of which are hereby incorporated by reference. The ability of an antigen binding moiety to bind to a specific antigenic determinant can be measured either through an enzyme-linked immunosorbent assay (ELISA) or other techniques familiar to one of skill in the art, e.g. surface plasmon resonance technique (analyzed on a BIACORE T100 system) (Liljeblad, et al., Glyco J 17, 323-329 (2000)), and traditional binding assays (Heeley. Endocr Res 28, 217-229 (2002)). Competition assays may be used to identify an antibody, antibody fragment, antigen binding domain or variable domain that competes with a reference antibody for binding to a particular antigen, e.g. an antibody that competes with the CH1A1A 98/99 2F1 antibody for binding to CEA. In certain embodiments, such a competing antibody binds to the same epitope (e.g. a linear or a conformational epitope) that is bound by the reference antibody. Detailed exemplary methods for mapping an epitope to which an antibody binds are provided in Morris (1996) “Epitope Mapping Protocols.” in Methods in Molecular Biology vol. 66 (Humana Press, Totowa, NJ). In an exemplary competition assay, immobilized antigen (e.g. CEA) is incubated in a solution comprising a first labeled antibody that binds to the antigen (e.g. CH1A1A 98/99 2F1 antibody) and a second unlabeled antibody that is being tested for its ability to compete with the first antibody for binding to the antigen. The second antibody may be present in a hybridoma supernatant. As a control, immobilized antigen is incubated in a solution comprising the first labeled antibody but not the second unlabeled antibody. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to the antigen. See Harlow and Lane (1988) Antibodies: A Laboratory Manual ch. 14 (Cold Spring Harbor Laboratory, Cold Spring Harbor, NY).

Antibodies described herein are preferably produced by recombinant means. Such methods are widely known in the state of the art and comprise protein expression in prokaryotic and eukaryotic cells with subsequent isolation of the antibody polypeptide and usually purification to a pharmaceutically acceptable purity. For the protein expression nucleic acids encoding light and heavy chains or fragments thereof are inserted into expression vectors by standard methods. Expression is performed in appropriate prokaryotic or eukaryotic host cells, such as CHO cells, NS0 cells, SP2/0 cells, HEK293 cells, COS cells, yeast, or E. coli cells, and the antibody is recovered from the cells (from the supernatant or after cells lysis).

Recombinant production of antibodies is well-known in the state of the art and described, for example, in the review articles of Makrides, S. C., Protein Expr. Purif. 17 (1999) 183-202; Geisse. S., et al., Protein Expr. Purif. 8 (1996) 271-282; Kaufman, R. J., Mol. Biotechnol. 16 (2000) 151-161: Werner, R. G., Drug Res. 48 (1998) 870-880.

The antibodies may be present in whole cells, in a cell lysate, or in a partially purified, or substantially pure form. Purification is performed in order to eliminate other cellular components or other contaminants, e.g. other cellular nucleic acids or proteins, by standard techniques, including alkaline/SDS treatment. CsCl banding, column chromatography, agarose gel electrophoresis, and others well known in the art. See Ausubel, F., et al., ed. Current Protocols in Molecular Biology, Greene Publishing and Wiley Interscience, New York (1987).

Expression in NS0 cells is described by, e.g., Barnes, L. M., et al., Cytotechnology 32 (2000) 109-123; Barnes, L. M., et al., Biotech. Bioeng. 73 (2001) 261-270. Transient expression is described by, e.g., Durocher, Y., et al., Nucl. Acids. Res. 30 (2002) E9. Cloning of variable domains is described by Orlandi, R., et al., Proc. Natl. Acad. Sci. USA 86 (1989) 3833-3837; Carter. P., et al., Proc. Natl. Acad. Sci. USA 89 (1992) 4285-4289; Norderhaug, L., et al., J. Immunol. Methods 204 (1997) 77-87. A preferred transient expression system (HEK 293) is described by Schlaeger. E.-J, and Christensen. K., in Cytotechnology 30 (1999) 71-83, and by Schlaeger. E.-J., in J. Immunol. Methods 194 (1996) 191-199.

The heavy and light chain variable domains according to the invention are combined with sequences of promoter, translation initiation, constant region. 3′ untranslated region, polyadenylation, and transcription termination to form expression vector constructs. The heavy and light chain expression constructs can be combined into a single vector, co-transfected, serially transfected, or separately transfected into host cells which are then fused to form a single host cell expressing both chains.

The control sequences that are suitable for prokaryotes, for example, include a promoter, optionally an operator sequence, and a ribosome binding site. Eukaryotic cells are known to utilize promoters, enhancers and polyadenylation signals.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The monoclonal antibodies are suitably separated from the culture medium by conventional immunoglobulin purification procedures such as, for example, protein A-Sepharose, hydroxylapatite chromatography, gel electrophoresis, dialysis, or affinity chromatography. DNA and RNA encoding the monoclonal antibodies are readily isolated and sequenced using conventional procedures. The hybridoma cells can serve as a source of such DNA and RNA. Once isolated, the DNA may be inserted into expression vectors, which are then transfected into host cells such as HEK 293 cells, CHO cells, or myeloma cells that do not otherwise produce immunoglobulin protein, to obtain the synthesis of recombinant monoclonal antibodies in the host cells.

As used herein, the expressions “cell”, “cell line”, and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included.

In the following statements, particular embodiments of the invention are described:
1. A recombinant Fc domain-IL2 variant (Fc-IL2v) polypeptide complex, comprising:
   (i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
   (ii) a second polypeptide comprising a variant CH2-CH3 region comprising G329 according to EU numbering.
2. The recombinant Fc-IL2v polypeptide complex according to embodiment 1 in combination with a recombinant membrane-anchored antigen binding (MAB) polypeptide or MAB polypeptide complex, for use in the treatment of cancer, for use in the prevention or treatment of metastasis, or for use in stimulating an immune response or function, such as T cell activity, wherein the MAB polypeptide or MAB polypeptide complex comprises an antigen-binding moiety, or a component thereof, and a transmembrane domain, wherein the antigen-binding moiety binds to a variant CH2-CH3 region comprising the amino acid substitution P329G according to EU numbering relative to the amino acid sequence of a reference CH2-CH3 region comprising P329 according to EU numbering, to which the antigen-binding moiety does not bind.
3. A method for treatment or prevention of cancer or for stimulating and immune response or function, such as T cell activity in an individual, wherein said method comprises
   (a) administration of a recombinant Fc domain-IL2 variant (Fc-IL2v) polypeptide complex to the individual, wherein the recombinant Fc-IL2v polypeptide complex comprises:
   (i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
   (ii) a second polypeptide comprising a variant CH2-CH3 region comprising G329 according to EU numbering; and
   (b) administration of a recombinant membrane-anchored antigen binding (MAB) polypeptide or MAB polypeptide complex, wherein the MAB polypeptide or MAB polypeptide complex comprises an antigen-binding moiety, or a component thereof, and a transmembrane domain, wherein the antigen-binding moiety binds to a variant CH2-CH3 region comprising the amino acid substitution P329G according to EU numbering, relative to the amino acid sequence of a reference CH2-CH3 region comprising P329 according to EU numbering, to which the antigen-binding moiety does not bind.
4. Use of a recombinant Fc domain-IL2 variant (Fc-IL2v) polypeptide complex in the manufacture of a medicament for treatment or prevention of cancer or for stimulating and immune response or function, such as T cell activity in an individual, wherein the recombinant Fc-IL2v polypeptide complex comprises:
   (i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
   (ii) a second polypeptide comprising a variant CH2-CH3 region comprising G329 according to EU numbering.
5. Use of a recombinant Fc domain-IL2 variant (Fc-IL2v) polypeptide complex in the manufacture of a medicament for the treatment or prevention of cancer or for stimulating and immune response or function, such as T cell activity in an individual, wherein the treatment comprises:
   (a) administration of a recombinant Fc domain-IL2 variant (Fc-IL2v) polypeptide complex to the individual, wherein the recombinant Fc-IL2v polypeptide complex comprises:
   (i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
   (ii) a second polypeptide comprising a variant CH2-CH3 region comprising G329 according to EU numbering; and
   (b) administration of a recombinant membrane-anchored antigen binding (MAB) polypeptide or MAB polypeptide complex, wherein the MAB polypeptide or MAB polypeptide complex comprises an antigen-binding moiety, or a component thereof, and a transmembrane domain, wherein the antigen-binding moiety binds to a variant CH2-CH3 region comprising the amino acid substitution P329G according to EU numbering, relative to the amino acid sequence of a reference CH2-CH3 region comprising P329 according to EU numbering, to which the antigen-binding moiety does not bind.
6. The recombinant Fc-IL2v polypeptide complex, method, or use of any one of the preceding embodiments, wherein the recombinant MAB is an antigen binding receptor or a component thereof, in particular wherein the recombinant MAB polypeptide is a chimeric antigen receptor (CAR) or a recombinant CD3-TCR complex.
7. The recombinant Fc-IL2v polypeptide complex, method, or use of any one of the preceding embodiments, wherein the antigen-binding moiety that binds to the Fc-IL2v comprises the heavy chain variable (VH) region and light chain variable (VL) region of an antibody that binds to the variant CH2-CH3 region comprising the amino acid substitution P329G according to EU numbering.
8. The recombinant Fc-IL2v polypeptide complex, method, or use of any one of the preceding embodiments, wherein the antigen-binding moiety is or comprises an Fv, scFv, Fab, Fab′, Fab-SH, F(ab′)₂, crossFab, scFab or dAb moiety.
9. The recombinant Fc-IL2v polypeptide complex, method, or use of any one of the preceding embodiments, wherein the antigen-binding moiety is or comprises an scFv.
10. The recombinant Fc-IL2v polypeptide complex, method, or use of any one of the preceding embodiments, wherein the component of the antigen-binding moiety is or comprises the heavy chain variable (VH) region or the light chain variable (VL) region of an antibody that binds to the variant CH2-CH3 region comprising the amino acid substitution P329G according to EU numbering.
11. The recombinant Fc-IL2v polypeptide complex, method, or use of any one of the preceding embodiments, wherein the antigen-binding moiety comprises a VL region incorporating the following CDRs:
   LC-CDR1 having the amino acid sequence of SEQ ID NO:24;
   LC-CDR2 having the amino acid sequence of SEQ ID NO:25; and
   LC-CDR3 having the amino acid sequence of SEQ ID NO:26.
12. The recombinant Fc-IL2v polypeptide complex, method, or use of any one of the preceding embodiments, wherein the antigen-binding moiety comprises a VH incorporating:
   (i) the following CDRs:
      HC-CDR1 having the amino acid sequence of SEQ ID NO:11;
      HC-CDR2 having the amino acid sequence of SEQ ID NO: 19; and
      HC-CDR3 having the amino acid sequence of SEQ ID NO: 13:
   or
   (ii) the following CDRs:
      HC-CDR1 having the amino acid sequence of SEQ ID NO:11;
      HC-CDR2 having the amino acid sequence of SEQ ID NO: 12; and
      HC-CDR3 having the amino acid sequence of SEQ ID NO: 13.
13. The recombinant Fc-IL2v polypeptide complex, method, or use of any one of the preceding embodiments, wherein the antigen-binding moiety comprises:
(a) (i) a VH region incorporating the following CDRs:
   HC-CDR1 having the amino acid sequence of SEQ ID NO: 11;
   HC-CDR2 having the amino acid sequence of SEQ ID NO: 19; and
   HC-CDR3 having the amino acid sequence of SEQ ID NO: 13:
   and
   (ii) a VL region incorporating the following CDRs:
   LC-CDR1 having the amino acid sequence of SEQ ID NO:24;
   LC-CDR2 having the amino acid sequence of SEQ ID NO:25; and
   LC-CDR3 having the amino acid sequence of SEQ ID NO:26;
or
(b) (i) a VH region incorporating the following CDRs:
   HC-CDR1 having the amino acid sequence of SEQ ID NO:11;
   HC-CDR2 having the amino acid sequence of SEQ ID NO: 12; and
   HC-CDR3 having the amino acid sequence of SEQ ID NO: 13:
   and
   (ii) a VL region incorporating the following CDRs:
   LC-CDR1 having the amino acid sequence of SEQ ID NO:24;
   LC-CDR2 having the amino acid sequence of SEQ ID NO:25; and
   LC-CDR3 having the amino acid sequence of SEQ ID NO:26.
14. The recombinant Fc-IL2v polypeptide complex, method, or use of any one of the preceding embodiments, wherein the antigen-binding moiety comprises a VL having an amino acid sequence having at least 70% amino acid sequence identity to SEQ ID NO:23.
15. The recombinant Fc-IL2v polypeptide complex, method, or use of any one of the preceding embodiments, wherein the antigen-binding moiety comprises a VH having an amino acid sequence having at least 70% amino acid sequence identity to SEQ ID NO:20, 18 or 10.
16. The recombinant Fc-IL2v polypeptide complex, method, or use of any one of the preceding embodiments, wherein the antigen-binding moiety comprises:
   (a) (i) a VH having an amino acid sequence having at least 70% amino acid sequence identity to SEQ ID NO:20; and
   (ii) a VL having an amino acid sequence having at least 70% amino acid sequence identity to SEQ ID NO:23;
or
   (b) (i) a VH having an amino acid sequence having at least 70% amino acid sequence identity to SEQ ID NO: 18; and
   (ii) a VL having an amino acid sequence having at least 70% amino acid sequence identity to SEQ ID NO:23;
or
   (c) (i) a VH having an amino acid sequence having at least 70% amino acid sequence identity to SEQ ID NO: 10; and
   (ii) a VL having an amino acid sequence having at least 70% amino acid sequence identity to SEQ ID NO:23.
17. The recombinant Fc-IL2v polypeptide complex, method, or use of any one of the preceding embodiments, wherein the recombinant MAB polypeptide comprises an amino acid sequence derived from IL2Ra, IL15Ra or CD8a.
18. The recombinant Fc-IL2v polypeptide complex, method, or use of any one of the preceding embodiments, wherein the recombinant MAB polypeptide comprises a transmembrane domain selected from the group consisting of the IL2Ra, IL15Ra and CD8a transmembrane domain or a fragment thereof capable of integrating into a plasma membrane.
19. The recombinant Fc-IL2v polypeptide complex, method, or use of any one of the preceding embodiments, wherein the recombinant MAB polypeptide comprises
   (i) an amino acid sequence derived from an amino acid sequence having at least 70% amino acid sequence identity to SEQ ID NOs: 61, 65, or 69;
   (ii) an amino acid sequence having at least 70% amino acid sequence identity to one of SEQ ID NOs: 63, 66, or 70; or
   (iii) an the amino acid sequence selected from the group consisting of SEQ ID NO:63, SEQ ID NO:67, and SEQ ID NO:71, or consist of a sequence thereof.
20. The recombinant Fc-IL2v polypeptide complex, method, or use of any one of the preceding embodiments, wherein the recombinant MAB polypeptide comprises a transmembrane domain selected from the group consisting of the CD8, the CD4, the CD3z, the FCGR3A, the NKG2D, the CD27, the CD28, the CD137, the OX40, the ICOS, the DAP10, the DAP12, or the CD40 transmembrane domain, or a fragment thereof capable of integrating into a plasma membrane.
21. The recombinant Fc-IL2v polypeptide complex, method, or use of any one of the embodiments 1-16, wherein the transmembrane domain is the CD8 transmembrane domain, in particular wherein the transmembrane domain comprises the amino acid sequence of SEQ ID NO:73 or a fragment thereof capable of integrating into a plasma membrane.
22. The recombinant Fc-IL2v polypeptide complex, method, or use of any one of the preceding embodiments, wherein the recombinant MAP polypeptide is a chimeric antigen receptor (CAR).
23. The recombinant Fc-IL2v polypeptide complex, method, or use of any one of the preceding embodiments, wherein the recombinant MAP polypeptide further comprises at least one stimulatory signaling domain and/or at least one co-stimulatory signaling domain.
24. The recombinant Fc-IL2v polypeptide complex, method, or use of embodiments 23, wherein the at least one stimulatory signaling domain is individually selected from the group consisting of the intracellular domain of CD3z, of FCGR3A and of NKG2D or fragments thereof that retain stimulatory signaling activity.
25. The recombinant Fc-IL2v polypeptide complex, method, or use of embodiments 23 or 22, wherein the at least one stimulatory signaling domain is the intracellular domain of CD3z or a fragment thereof that retains stimulatory signaling activity, in particular wherein the at least one stimulatory signaling domain comprises the amino acid sequence of SEQ ID NO: 75 or a fragment thereof that retain stimulatory signaling activity.
26. The recombinant Fc-IL2v polypeptide complex, method, or use of any one of embodiments 23-25, wherein the at least one co-stimulatory signaling domain is individually selected from the group consisting of the intracellular domain of CD27, of CD28, of CD137, of OX40, of ICOS, of DAP10, of DAP12, and of CD40 or fragments thereof that retain co-stimulatory signaling activity.
27. The recombinant Fc-IL2v polypeptide complex, method, or use of any one of embodiments 23-26, wherein the at least one co-stimulatory signaling domain is the CD137 intracellular domain or a fragment thereof that retains CD137 co-stimulatory activity, in particular wherein the co-stimulatory signaling domain comprises the amino acid sequence of SEQ ID NO:76 or a fragment thereof that retain co-stimulatory signaling activity.
28. The recombinant Fc-IL2v polypeptide complex, method, or use of any one of embodiments 23-27, wherein the at least one co-stimulatory signaling domain is the CD28 intracellular domain or a fragment thereof that retains CD28 co-stimulatory activity, in particular wherein the co-stimulatory signaling domain comprises the amino acid sequence of SEQ ID NO:74 or a fragment thereof that retain co-stimulatory signaling activity.
29. The recombinant Fc-IL2v polypeptide complex, method, or use of any one of embodiments 23-28, wherein the CAR comprises a stimulatory signaling domain comprising the intracellular domain of CD3z or a fragment thereof that retains CD3z stimulatory signaling activity, and wherein the CAR comprises a co-stimulatory signaling domain comprising the intracellular domain of CD28 or a fragment thereof that retains CD28 co-stimulatory signaling activity.
30. The recombinant Fc-IL2v polypeptide complex, method, or use of any one of embodiments 23-28, wherein the CAR comprises a stimulatory signaling domain comprising the intracellular domain of CD3z or a fragment thereof that retains CD3z stimulatory signaling activity, and wherein the CAR comprises a co-stimulatory signaling domain comprising the intracellular domain of CD137 or a fragment thereof that retains CD137 co-stimulatory signaling activity.
31. The recombinant Fc-IL2v polypeptide complex, method, or use of any one of embodiments 23-30, wherein (i) the stimulatory signaling domain comprises the amino acid sequence of SEQ ID NO: 75 or a fragment thereof that retains stimulatory signaling activity, and wherein the co-stimulatory signaling domain comprises the amino acid sequence of SEQ ID NO:74 or a fragment thereof that retains co-stimulatory signaling activity, or (ii) the stimulatory signaling domain comprises the amino acid sequence of SEQ ID NO:75 or a fragment thereof that retains stimulatory signaling activity, and wherein the co-stimulatory signaling domain comprises the amino acid sequence of SEQ ID NO:76 or a fragment thereof that retains co-stimulatory signaling activity
32. The recombinant Fc-IL2v polypeptide complex, method, or use of any one of the preceding embodiments, wherein the antigen binding moiety is connected to the transmembrane domain, optionally through a peptide linker.
33. The recombinant Fc-IL2v polypeptide complex, method, or use of any one of the preceding embodiments, wherein the peptide linker comprises the amino acid sequence of SEQ ID NO:80.
34. The recombinant Fc-IL2v polypeptide complex, method, or use of any one of embodiments 23-31, wherein the transmembrane domain is connected to the co-signaling domain or to the stimulatory signaling domain, optionally through a peptide linker.
35. The recombinant Fc-IL2v polypeptide complex, method, or use of any one of embodiments 23-32, wherein the signaling and/or co-signaling domains are connected, optionally through at least one peptide linker.
36. The recombinant Fc-IL2v polypeptide complex, method, or use of any one of the preceding embodiments, wherein the antigen binding moiety is connected at the C-terminus to the N-terminus of the transmembrane domain, optionally through a peptide linker.
37. The recombinant Fc-IL2v polypeptide complex, method, or use of any one of the preceding embodiments, wherein the light chain variable domain (VL) is connected at the C-terminus to the N-terminus of the transmembrane, optionally through a peptide linker.
38. The recombinant Fc-IL2v polypeptide complex, method, or use of any one of the preceding embodiments, wherein the heavy chain variable domain (VH) is connected at the C-terminus to the N-terminus of the light chain variable domain (VL), optionally through a peptide linker.
39. The recombinant Fc-IL2v polypeptide complex, method, or use of any one of embodiments 22-38, wherein the CAR comprises one co-signaling domain, wherein the co-signaling domain is connected at the N-terminus to the C-terminus of the transmembrane domain.
40. The recombinant Fc-IL2v polypeptide complex, method, or use of any one of embodiments 22-39, wherein the CAR comprises one stimulatory signaling domain, wherein the stimulatory signaling domain is connected at the N-terminus to the C-terminus of the co-stimulatory signaling domain.
41. The recombinant Fc-IL2v polypeptide complex, method, or use of any one of the embodiments 22-40, wherein the CAR comprises an amino acid sequence that is at least about 95%, 96%, 97%, 98%, 99% or 100% identical to the amino acid sequence of SEQ ID NO: 146, 149, 151 or 154.
42. The recombinant Fc-IL2v polypeptide complex, method, or use of any one of the preceding embodiments, wherein the recombinant MAP polypeptide comprises at least one recombinant CD3-TCR complex polypeptide.
43. The recombinant Fc-IL2v polypeptide complex, method, or use of any one of embodiment 42, wherein the recombinant CD3-TCR complex polypeptide comprises:
   (i) an antigen-binding moiety, or a component thereof, wherein the antigen-binding moiety binds to the variant CH2-CH3 region comprising the amino acid substitution P329G according to EU numbering, relative to the amino acid sequence of a reference CH2-CH3 region comprising P329 according to EU numbering, to which the antigen-binding moiety does not bind; and
   (ii) a CD3-TCR complex association domain having an amino acid sequence derived from a CD3-TCR complex polypeptide.
44. The recombinant Fc-IL2v polypeptide complex, method, or use of embodiments 43, wherein the recombinant CD3-TCR complex polypeptide is capable of associating through its CD3-TCR complex association domain with one or more CD3-TCR complex polypeptides to form a CD3-TCR complex.
45. The recombinant Fc-IL2v polypeptide complex, method, or use of embodiment 43 or 44, wherein the amino acid sequence derived from a CD3-TCR complex polypeptide is derived from CD3ε, TCRα or TCRβ.
46. The recombinant Fc-IL2v polypeptide complex, method, or use of any one of embodiments 43-45, wherein the CD3-TCR complex association domain comprises, or consists of, an amino acid sequence having at least 70% amino acid sequence identity to one of SEQ ID NOs: 157, 161, 165, 186, 208, 209, or 210.
47. The recombinant Fc-IL2v polypeptide complex, method, or use of any one of embodiments 43-46, wherein the amino acid sequence derived from a CD3-TCR complex polypeptide is derived from CD38.
48. The recombinant Fc-IL2v polypeptide complex, method, or use of any one of embodiments 43-47, wherein the CD3-TCR complex association domain comprises, or consists of, an amino acid sequence having at least 70% amino acid sequence identity to SEQ ID NO: 186.
49. The recombinant Fc-IL2v polypeptide complex, method, or use of any one of embodiments 43-48, wherein the amino acid sequence derived from a CD3-TCR complex polypeptide is derived from TCRα or TCRβ.
50. The recombinant Fc-IL2v polypeptide complex, method, or use of any one of embodiments 43-49, wherein the CD3-TCR complex association domain comprises, or consists of, an amino acid sequence having at least 70% amino acid sequence identity to one of SEQ ID NOs: 157, 161, 165, 208, 209, or 210.
51. The recombinant Fc-IL2v polypeptide complex, method, or use of any one of embodiments 43-50, wherein the antigen-binding moiety or component thereof is connected at its C-terminus to the N-terminus of the CD3-TCR complex association domain, optionally through a linker sequence.
52. The recombinant Fc-IL2v polypeptide complex, method, or use of any one of embodiments 42-51, wherein the recombinant MAB polypeptide comprises:
   (a) a first recombinant CD3-TCR complex polypeptide comprising:
      (i) a first component of an antigen-binding moiety, wherein the antigen-binding moiety binds to a variant Fc domain having an amino acid sequence comprising at least one amino acid difference relative to a reference Fc domain to which the antigen-binding moiety does not bind; and
      (ii) a CD3-TCR complex association domain having an amino acid sequence derived from a CD3-TCR complex polypeptide; and
   (b) a second recombinant CD3-TCR complex polypeptide comprising:
      (i) a second component of the antigen-binding moiety of (a) (i); and
      (ii) a CD3-TCR complex association domain having an amino acid sequence derived from a CD3-TCR complex polypeptide:
   wherein the first and second recombinant CD3-TCR complex polypeptides are capable of associating through their CD3-TCR complex association domains to form the antigen-binding moiety.
53. The recombinant Fc-IL2v polypeptide complex, method, or use of embodiments 52, wherein the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain.
54. The recombinant Fc-IL2v polypeptide complex, method, or use of embodiment 52 or embodiment 53, wherein:
   (i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
   (ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.
55. The recombinant Fc-IL2v polypeptide complex, method, or use of embodiment 54, wherein the CD3-TCR complex association domain derived from TCRα comprises, or consists of, an amino acid sequence having at least 70% amino acid sequence identity to SEQ ID NO: 157 or 208.
56. The recombinant Fc-IL2v polypeptide complex, method, or use of embodiment 54 or embodiment 55, wherein the CD3-TCR complex association domain derived from TCRβ comprises, or consists of, an amino acid sequence having at least 70% amino acid sequence identity to one of SEQ ID NOs: 161, 165, 209, or 210.
57. The recombinant Fc-IL2v polypeptide complex, method, or use of any one of embodiments 42-56, wherein the recombinant MAB polypeptide comprises:
   (a) a first recombinant CD3-TCR complex polypeptide comprising:
      (i) a first component of an antigen-binding moiety, wherein the antigen-binding moiety binds to a variant Fc domain having an amino acid sequence comprising at least one amino acid difference relative to a reference Fc domain to which the antigen-binding moiety does not bind, wherein the variant Fc domain comprises a CH2-CH3 region comprising G329 according to EU numbering; and
      (ii) a CD3-TCR complex association domain having an amino acid sequence derived from a CD3-TCR complex polypeptide; and
   (b) a second recombinant CD3-TCR complex polypeptide comprising:
      (i) a second component of the antigen-binding moiety of (a) (i); and
      (ii) a CD3-TCR complex association domain having an amino acid sequence derived from a CD3-TCR complex polypeptide;
   wherein the first and second recombinant CD3-TCR complex polypeptides are capable of associating through their CD3-TCR complex association domains to form the antigen-binding moiety:
   wherein the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain:
   and wherein:
   (i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
   (ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.
58. The recombinant Fc-IL2v polypeptide complex, method, or use of any one of the preceding embodiments, wherein the first and the second polypeptide comprising a CH2-CH3 region comprising G329 according to EU numbering comprise a modification in the CH3 domain of the CH2-CH3 region promoting the association of the first and the second polypeptide comprising a CH2-CH3 region comprising G329 according to EU numbering.
59. The recombinant Fc-IL2v polypeptide complex, method, or use of any one of embodiment 58, wherein in the CH3 domain of the first polypeptide an amino acid residue is replaced with an amino acid residue having a larger side chain volume, thereby generating a protuberance within the CH3 domain of the first polypeptide which is positionable in a cavity within the CH3 domain of the second polypeptide, and in the CH3 domain of the second polypeptide an amino acid residue is replaced with an amino acid residue having a smaller side chain volume, thereby generating a cavity within the CH3 domain of the second polypeptide within which the protuberance within the CH3 domain of the first polypeptide is positionable.
60. The recombinant Fc-IL2v polypeptide complex, method, or use of any one of the preceding embodiment, wherein in the first polypeptide the threonine residue at position 366 is replaced with a tryptophan residue (T366W), and in the second polypeptide the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to EU numbering).
61. The recombinant Fc-IL2v polypeptide complex, method, or use of any one of the preceding embodiment, wherein in the first polypeptide additionally the serine residue at position 354 is replaced with a cysteine residue (S354C) or the glutamic acid residue at position 356 is replaced with a cysteine residue (E356C), and in the second polypeptide additionally the tyrosine residue at position 349 is replaced by a cysteine residue (Y349C) (numberings according to EU numbering).
62. The recombinant Fc-IL2v polypeptide complex, method, or use of any one of the preceding embodiment, wherein the IL2v polypeptide is fused at its amino-terminal amino acid to the carboxy-terminal amino acid of the first polypeptide, optionally through a linker peptide.
63. The recombinant Fc-IL2v polypeptide complex, method, or use of any one of the preceding embodiment, wherein the linker peptide has the amino acid sequence of SEQ ID NO:32.
64. The recombinant Fc-IL2v polypeptide complex, method, or use of any one of the preceding embodiment, wherein the first and/or second polypeptide comprising a variant CH2-CH3 region comprising G329 according to EU numbering comprises one or more further amino acid substitution that reduce binding to an Fc receptor, particularly an Fcγ receptor, and/or effector function, particularly antibody-dependent cell-mediated cytotoxicity (ADCC).
65. The recombinant Fc-IL2v polypeptide complex, method, or use of embodiment 64, wherein said one or more amino acid substitution is L234A and/or L235A according to EU numbering.
66. The recombinant Fc-IL2v polypeptide complex, method, or use of any one of the preceding embodiments, wherein the mutant IL-2 polypeptide:
   (i) has reduced binding affinity to the α-subunit of the IL-2 receptor (as compared to wild-type IL-2, in particular human IL-2 shown as SEQ ID NO: 40),
   (ii) comprises one, two or three amino acid substitution(s) at one, two or three position(s) selected from the positions corresponding to residues 42, 45 and 72 of human IL-2 shown as SEQ ID NO: 40, in particular the three specific amino acid substitutions F42A, Y45A and L72G,
   (iii) comprises the features as set out in (ii) plus an amino acid substitution at a position corresponding to residue 3 of human IL-2 shown as SEQ ID NO: 40, in particular the specific amino acid substitution T3A,
   (iv) four amino acid substitutions at positions corresponding to residues 3, 42, 45 and 72 of human IL-2 shown as SEQ ID NO: 40, in particular the specific amino acid substitutions T3A, F42A, Y45A and L72G, and/or
   (v) comprises the amino acid substitution T3A and/or the amino acid substitution C125A.
67. The recombinant Fc-IL2v polypeptide complex, method, or use of any one of the preceding embodiments, wherein the mutant IL-2 polypeptide further comprises the amino acid substitution Q126T.
68. The recombinant Fc-IL2v polypeptide complex, method, or use of any one of the preceding embodiments, wherein the mutant IL-2 polypeptide comprises the sequence of SEQ ID NO: 38 or 39.
69. The recombinant Fc-IL2v polypeptide complex, method, or use of any one of the preceding embodiments, wherein the Fc-IL2v comprises not more than one mutant IL-2 polypeptide.
70. The recombinant Fc-IL2v polypeptide complex, method, or use of any one of the preceding embodiments, wherein the Fc-IL2v comprises an Fc domain composed of a first and a second subunit.
71. The recombinant Fc-IL2v polypeptide complex, method, or use of embodiment 70, wherein the Fc domain is an IgG class, particularly an IgG₁ subclass. Fc domain.
72. The recombinant Fc-IL2v polypeptide complex, method, or use of embodiment 70 or 71, wherein the Fc domain is a human Fc domain.
73. The recombinant Fc-IL2v polypeptide complex, method, or use any one of the preceding embodiments, wherein the recombinant Fc-IL2v polypeptide complex does not comprise an antigen binding moiety, in particular wherein the recombinant Fc-IL2v polypeptide complex does not comprise a scFv, Fab or crossFab.
74. The recombinant Fc-IL2v polypeptide complex, method, or use any one of the preceding embodiments, wherein the recombinant Fc-IL2v polypeptide complex comprises:
   (i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
   (ii) a second polypeptide comprising a variant CH2-CH3 region comprising G329 according to EU numbering.
wherein the recombinant Fc-IL2v polypeptide complex does not comprise an antigen binding moiety.

In some embodiments, the recombinant Fc-IL2v polypeptide complex according to the present disclosure comprises:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40, wherein the first polypeptide comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to an amino acid sequence selected from the group consisting of SEQ ID NO:43, SEQ ID NO:44, SEQ ID NO:47, and SEQ ID NO:48; and
(ii) a second polypeptide comprising a variant CH2-CH3 region comprising G329 according to EU numbering, wherein the second polypeptide comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:42.
   wherein the recombinant Fc-IL2v polypeptide complex does not comprise an antigen binding moiety, wherein the first and second polypeptide are capable of stable association.
75. The recombinant Fc-IL2v polypeptide complex, method, or use any one of the preceding embodiments, wherein the recombinant Fc-IL2v polypeptide complex comprises:
   (i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40, wherein the first polypeptide comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to an amino acid sequence selected from the group consisting of SEQ ID NO:50, SEQ ID NO:51, SEQ ID NO:52 and SEQ ID NO:53 and
   (ii) a second polypeptide comprising a variant CH2-CH3 region comprising G329 according to EU numbering, wherein the second polypeptide comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:41.
wherein the recombinant Fc-IL2v polypeptide complex does not comprise an antigen binding moiety, wherein the first and second polypeptide are capable of stable association.
76. The recombinant Fc-IL2v polypeptide complex, method, or use any one of the preceding embodiments, wherein the recombinant Fc-IL2v polypeptide complex comprises:
   (i) a first polypeptide comprising an amino acid sequence selected from the group consisting of SEQ ID NO:43, SEQ ID NO:44, SEQ ID NO:47, and SEQ ID NO: 48; and
   (ii) a second polypeptide comprising the amino acid sequence of SEQ ID NO:42, wherein the recombinant Fc-IL2v polypeptide complex does not comprise an antigen binding moiety.
77. The recombinant Fc-IL2v polypeptide complex, method, or use any one of the preceding embodiments, wherein the recombinant Fc-IL2v polypeptide complex according to the present disclosure comprises:
   (i) a first polypeptide comprising an amino acid sequence selected from the group consisting of SEQ ID NO:50, SEQ ID NO:51, SEQ ID NO:52 and SEQ ID NO:53; and
   (ii) a second polypeptide comprising the amino acid sequence of SEQ ID NO:41, wherein the recombinant Fc-IL2v polypeptide complex does not comprise an antigen binding moiety.
78. The recombinant Fc-IL2v polypeptide complex, method, or use any one of the preceding embodiments, wherein the recombinant Fc-IL2v polypeptide complex comprises:
   (i) a first polypeptide consisting of an amino acid sequence selected from the group consisting of SEQ ID NO:43, SEQ ID NO:44, SEQ ID NO:47, and SEQ ID NO:48; and
   (ii) a second polypeptide consisting of the amino acid sequence of SEQ ID NO:42.
79. The recombinant Fc-IL2v polypeptide complex, method, or use any one of the preceding embodiments, wherein the recombinant Fc-IL2v polypeptide complex comprises:
   (i) a first polypeptide consisting of an amino acid sequence selected from the group consisting of SEQ ID NO:50, SEQ ID NO:51, SEQ ID NO:52 and SEQ ID NO:53; and
   (ii) a second polypeptide consisting of the amino acid sequence of SEQ ID NO:41, wherein the recombinant Fc-IL2v polypeptide complex does not comprise an antigen binding moiety.
80. The recombinant Fc-IL2v polypeptide complex, method, or use any one of the preceding embodiments, wherein the recombinant Fc-IL2v polypeptide complex consists of:
   (i) a first polypeptide consisting of an amino acid sequence selected from the group consisting of SEQ ID NO:43, SEQ ID NO:44, SEQ ID NO:47, and SEQ ID NO:48; and
   (ii) a second polypeptide consisting of the amino acid sequence of SEQ ID NO:42.
81. The recombinant Fc-IL2v polypeptide complex, method, or use any one of the preceding embodiments, wherein the recombinant Fc-IL2v polypeptide complex consists of:
   (i) a first polypeptide consisting of an amino acid sequence selected from the group consisting of SEQ ID NO:50, SEQ ID NO:51, SEQ ID NO:52 and SEQ ID NO:53; and
   (ii) a second polypeptide consisting of the amino acid sequence of SEQ ID NO:41, wherein the recombinant Fc-IL2v polypeptide complex does not comprise an antigen binding moiety.
82. The recombinant Fc-IL2v polypeptide complex, method, or use any one of the preceding embodiments, wherein the recombinant Fc-IL2v polypeptide complex according to the present disclosure consists of:
   (i) a first polypeptide consisting of the amino acid sequence of SEQ ID NO:43 or SEQ ID NO: 44; and
   (ii) a second polypeptide consisting of the amino acid sequence of SEQ ID NO:42.
83. The recombinant Fc-IL2v polypeptide complex, method, or use of any one of embodiments 1-72, wherein the recombinant Fc-IL2v polypeptide complex further comprises an antibody that binds to PD-1.
84. The recombinant Fc-IL2v polypeptide complex, method, or use of embodiment 83, wherein the antibody comprises three heavy chain complementary determining regions (CDRs) (CDR-H1, CDR-H2 and, CDR-H3) contained within a heavy chain variable region (VH) comprising the amino acid sequence of SEG ID NO: 36, and three light chain complementary determining regions (CDRs) (CDR-L1, CDR-L2 and, CDR-L3) contained within a light chain variable region (VL) comprising the amino acid sequence of SEG ID NO: 37.
85. The recombinant Fc-IL2v polypeptide complex, method, or use of embodiments 83 or 84, wherein the antibody comprises comprises (a) a heavy chain variable region (VH) comprising an amino acid sequence that is at least about 95%, 96%, 97%, 98%, 99% or 100% identical to the amino acid sequence of SEQ ID NO: 36, and (b) a light chain variable region (VL) comprising an amino acid sequence that is at least about 95%, 96%, 97%, 98%, 99% or 100% identical to the amino acid sequence of SEQ ID NO:37.
86. The recombinant Fc-IL2v polypeptide complex, method, or use of any one of embodiments 83-85, wherein the antibody comprises wherein the antibody comprises (a) a heavy chain variable region (VH) comprising the amino acid sequence of SEQ ID NO:36, and (b) a light chain variable region (VL) comprising the amino acid sequence of SEQ ID NO:37.
87. The recombinant Fc-IL2v polypeptide complex, method, or use of any one of embodiments 1-72, wherein the recombinant Fc-IL2v polypeptide complex comprises or consist of a polypeptide comprising an amino acid sequence that is at least about 80%, 85%, 90%, 95%, 96%, 97%, 98%, 99% or 100% identical to the sequence of SEQ ID NO:322, a polypeptide comprising an amino acid sequence that is at least about 80%, 85%, 90%, 95%, 96%, 97%, 98%, 99% or 100% identical to the sequence of SEQ ID NO:323, a polypeptide comprising an amino acid sequence that is at least about 80%, 85%, 90%, 95%, 96%, 97%, 98%, 99% or 100% identical to the sequence of SEQ ID NO:324 and a polypeptide comprising an amino acid sequence that is at least about 80%, 85%, 90%, 95%, 96%, 97%, 98%, 99% or 100% identical to the sequence of SEQ ID NO:325.
88. The recombinant Fc-IL2v polypeptide complex, method, or use of any one of embodiments 1-72, wherein the recombinant Fc-IL2v polypeptide complex comprises or consist of a polypeptide comprising an amino acid sequence that is at least about 80%, 85%, 90%, 95%, 96%, 97%, 98%, 99% or 100% identical to the sequence of SEQ ID NO:326, a polypeptide comprising an amino acid sequence that is at least about 80%, 85%, 90%, 95%, 96%, 97%, 98%, 99% or 100% identical to the sequence of SEQ ID NO: 327, and a polypeptide comprising an amino acid sequence that is at least about 80%, 85%, 90%, 95%, 96%, 97%, 98%, 99% or 100% identical to the sequence of SEQ ID NO:328.89. A nucleic acid, or a plurality of nucleic acids, encoding a recombinant Fc domain-IL2 variant (Fc-IL2v) polypeptide complex according to any one of embodiments 1 to 86, or recombinant MAB polypeptide or MAB polypeptide complex according to any one of embodiments 2 to 86.
90. An expression vector, or a plurality of expression vectors, comprising a nucleic acid according to embodiment 89.
91. A cell comprising a recombinant MAB polypeptide or MAB polypeptide complex according to any one of embodiments 2 to 87, a nucleic acid or a plurality of nucleic acids according to embodiment 89, or an expression vector or a plurality of expression vectors according to embodiment 88.
92. The recombinant Fc-IL2v polypeptide complex, method, or use of any one of embodiments 2-87, or the cell of embodiment 91, wherein cells expressing the recombinant MAB polypeptide and/or the recombinant MAB polypeptide complex cells are specifically expanded, in particular wherein the cells are specifically expanded by contacting the cells with the recombinant Fc-IL2v polypeptide complex of any one of embodiments 1-87.
93. The recombinant Fc-IL2v polypeptide complex, method, or use of any one of embodiments 2-87 or 92, or the cell of embodiment 91, wherein cells expressing the recombinant MAB polypeptide and/or the recombinant MAB polypeptide complex cells are enriched, in particular wherein the cells are enriched by contacting the cells with the recombinant Fc-IL2v polypeptide complex of any one of embodiments 1-87.
94. The recombinant Fc-IL2v polypeptide complex, method, or use of any one of embodiments 2-87 or 92-93, or the cell of embodiment 89, wherein cells expressing the recombinant MAB polypeptide and/or the recombinant MAB polypeptide complex cells are enriched to >90% of a total cell pool.
95. A method of producing an enriched pool of cells comprising contacting a starting pool of cells comprising at least one cell according to embodiment 91 with the recombinant Fc-IL2v polypeptide complex of any one of embodiments 1-86 and incubating the cells until the fraction of cells comprising the recombinant MAB polypeptide or MAB polypeptide complex reaches a desired fraction of the total pool of cells to produce the enriched pool of cells.
96. A pharmaceutical composition comprising a cell according to any one of embodiments 91-93 or an enriched pool of cells produced according to embodiment 95.
97. A pharmaceutical composition comprising a recombinant Fc domain-IL2 variant (Fc-IL2v) polypeptide complex according to any one of embodiments 1 to 81.
98. The invention as hereinbefore described with reference to the Figures and Examples.

### EXAMPLES

The following are examples of methods and compositions of the invention. It is understood that various other embodiments may be practiced, given the general description provided above.

### Example 1

### 1.1 Recombinant DNA Techniques

Standard methods were used to manipulate DNA as described in Sambrook et al., Molecular cloning: A laboratory manual: Cold Spring Harbor Laboratory Press, Cold Spring Harbor, New York, 1989. The molecular biological reagents were used according to the manufacturers' instructions. General information regarding the nucleotide sequences of human immunoglobulins light and heavy chains is given in: Kabat, E. A, et al., (1991) Sequences of Proteins of Immunological Interest, 5^{th} ed., NIH Publication No. 91-3242.

### 1.2 DNA Sequencing

DNA sequences were determined by double strand Sanger sequencing.

### 1.3 Gene Synthesis

Desired gene segments where required were either generated by PCR using appropriate templates or were synthesized by Genscript Biotech (New Jersey, US) or GeneArt (Thermo Fisher Scientific, Regensburg, Germany) from synthetic oligonucleotides and PCR products by automated gene synthesis. The gene segments flanked by single restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

### 1.4 Production of IgG-Like Proteins in Expi293F Cells

Antibodies and antibody-cytokine fusion proteins were generated by transient transfection of Expi293F cells. Cells were seeded in Expi293 media (Gibco, #1435101) at a density of 2.5×10⁶/ml. Expression vectors and ExpiFectamine (Gibco, ExpiFectamine transfection kit, #13385544) were separately mixed in OptiMEM (Gibco, #11520386). After 5 min, both solutions were combined, mixed by pipetting and incubated for 25 minutes at room temperature. Cells were added to the vector/ExpiFectamine solution and incubated for 24 hours at 37° C., in a shaking incubator with a 5% CO2 atmosphere. One day post transfection, supplements (Enhancer 1+2, ExpiFectamine transfection kit) were added. Cell supernatants were harvested after 4-5 days by centrifugation and subsequent filtration (0.2 μm filter), and proteins were purified from the harvested supernatant by standard methods as indicated below.

### 1.5 Purification of IgG-Like Proteins

Proteins were purified from filtered cell culture supernatants referring to standard protocols. In brief. Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate PH neutralization of the sample. The protein was concentrated by centrifugation (Millipore Amicon® ULTRA-15, #UFC903096), and aggregated protein was separated from monomeric protein by size exclusion chromatography in 20 mM histidine. 140 mM sodium chloride, pH 6.0.

### 1.6 Production of IgG-Like Proteins in CHO K1 Cells

Alternatively, the antibodies and antibody-cytokine fusion proteins described herein were prepared by Evitria using their proprietary vector system with conventional (non-PCR based) cloning techniques and using suspension-adapted CHO K1 cells (originally received from ATCC and adapted to serum-free growth in suspension culture at Evitria). For the production. Evitria used its proprietary, animal-component free and serum-free media (eviGrow and eviMake2) and its proprietary transfection reagent (eviFect). Supernatant was harvested by centrifugation and subsequent filtration (0.2 μm filter) and afterwards purified from the harvested supernatant by standard methods.

### 1.7 Analytics of IgG-Like Proteins

The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer). Determination of the aggregate content was performed by HPLC chromatography at 25° C., using analytical size-exclusion column (TSKgel G3000 SW XL or UP-SW3000) equilibrated in running buffer (200 mM KH2PO4, 250 mM KCl pH 6.2, 0.02% NaN3).

### 1.8 Preparation of Virus Like Particles (VLPs)

Lipofectamine LTX™-based transfection was performed using ˜70% confluent Lenti-X™ 293T cells (Takara, #632180) and the construct encoding transfer vectors as well as packaging vectors pCAG-VSVG and psPAX2 at a 2:1:2 molar ratio (Giry-Laterriere M, et al Methods Mol Biol. 2011:737:183-209, Myburgh R, et al Mol Ther Nucleic Acids. 2014). As control for every experiment, mock virus-like particles (VLPs) using only the packaging vectors, but no transfer vector, were produced. After 48 hours, the supernatant was collected and centrifuged for 5 minutes at 350×g to remove remaining cells and purify the virus particles. VLPs were used directly or concentrated 10-fold (Lenti-x-Concentrator, Takara, #631231). For storage the VLPs were aliquoted in Eppendorf tubes and snap frozen in liquid nitrogen, before being stored at −80° C.

### 1.9 Transduction CTLL-2

CTLL-2 cells were seeded in a 24-well plate (0.75 mio/well in 1 ml) in RPMI (Gibco, 42401-018), 10% FBS (Sigma, #F4135-500ML), 1× Glutamax (Gibco, #35050-038) and 10% T-STIM with CON A (Corning, #354115). VLPs were used fresh or thawed at 37° C., and 300 μl were added together with 8 μg/ml Polybrene (Sigma Aldrich) and Lentiboost P (1:100) (Sirion Biotech, #SB-P-LV-101-12) to the cells in a 24-well plate for spinfection at 1100× g for 99 min and 31° C. The cells were incubated for at least 72 hours at 37° C. 5% CO2, before the transduction was checked by flow cytometry.

1.10 Isolation of Primary T Cells from Buffy Coats

Buffy coats were ordered from Blutspende Zürich (Rütistrasse 19, 8952 Schlieren). A Leucosep tube with 15 mL of room temperature Histopaque density gradient medium (Sigma-Aldrich, #10771) was prepared and centrifuged at 400×g for 5 minutes, until the Histopaque passed the filter. The blood was transferred to a T75 flask and an equal volume of DPBS was added. 30 ml of the blood/buffer mixture was added to the Leucosep Tubes and they were centrifuged 1200×g for 20 minutes with the breaks off. The band containing the peripheral blood mononuclear cells (PBMCs) was carefully pipetted into a fresh 50 ml falcon tube and topped up to 50 ml with DPBS. The tubes were centrifuged at 300×g for 10 minutes, then the supernatant was discarded. This step was repeated two more times, before the cells were resuspended in DPBS and counted. Pan T cell isolation was performed by negative selection according to the manufacturer's instructions using the Pan T cell isolation kit (Miltenyi, #130-096-535). The cells were either frozen or used directly after isolation. Cells were cultured in advanced RPMI (Gibco, #11530446), 10% FBS (Sigma, #F4135-500ML), 1% Glutamax (Gibco, #35050-038), 50 IU/Proleukin (Novartis), 25 ng/ml IL-7 (Miltenyi, #130-095-364) and 50 ng/ml IL-15 (Miltenyi, #130-095-766) (T cell medium).

### 1.11 Transduction of Primary T Cells

T cells were activated for 16-24 hours using ImmunoCult™ Human CD3/CD28/CD2 T Cell Activator (StemCell, #10990) according to the manufacturer's manual. Then the activated cells were resuspended and counted and 1.5 million cells/well were seeded in a 24-well plate. VLPs were used fresh or thawed at 37° C., and 150-300 μl were added together with 8 μg/ml Polybrene (Sigma Aldrich) and Lentiboost P (1:100) (Sirion Biotech. #SB-P-LV-101-12) to the cells in a 24-well plate for spinfection at 1100×g for 99 min and 31° C. If necessary a gene knockout was performed 24 hours after the transduction. The cells were incubated for at least 72 hours at 37° C., 5% CO2, before the transduction was checked by flow cytometry.

### 1.12 CRISPR/Cas9 Mediated Knockout in Transduced Primary T Cells

For single CRISPR KOs, ribonucleoprotein complexes (RNPs) were prepared by carefully mixing 2 μl Cas9 (TrueCut Cas9, Invitrogen, #A36499) with 3 μl single sgRNA (100 uM, Integrated DNA Technologies (IDT)). For double knockouts 1 μl Cas9 was mixed with 1.5 μl of single sgRNA_1 and 1 μl Cas9 was mixed with 1.5 μl of single sgRNA_2. The mixtures were incubated for 10 minutes at room temperature and in case of a double KO, the two separately formed RNPs were mixed together after the incubation. 24 hours after transduction, one million primary T cells were spun down (350×g, 3 min) and washed once with DPBS. The cell pellet was resuspended in 20 μl P3 Primary Cell Nucleofector™ Solution (Lonza, #V4XP-3024), which was prior adapted to room temperature. The RNPs were added to the cell suspension, mixed and transferred to a well of an electroporation stripe. Electroporation with the 4D-Nucleofector unit (Lonza) was performed using pulse code EH-115. Then the cells were resuspended in prewarmed T cell medium and incubated at 37° C., 5% CO2 until the next steps (at least 3 days).

### 1.13 CellTiter-Glo Viability Assay

The required amount of CTLL-2 cells was washed three times with DPBS to remove residual IL-2 (5 min, 280×g). The cells were resuspended in assay medium (RPMI, 10% FBS, 1% Glutamax) and incubated for 3 hours in the incubator (37° C., 5% CO2) for starvation. The cell number was adjusted to 0.2 mio/ml and 50 μl were seeded in a 96-well U-bottom plate (Greiner, #650185), resulting in 10,000 cells/well. 50 μl of the diluted compounds were added to the cells and the plate was incubated for 72 hours at 37° C. and 5% CO2. For the readout the assay plate and the CellTiter-Glo reagent (Promega, #G7571/2/3) were equilibrated to room temperature for around 30 to 60 minutes. Then 100 μl of the reagent were added to each well of the assay plate and the plate was incubated for 10 minutes on the cell shaker protected from light. 150 μl of the mixture were transferred to a white flat bottom 96-well plate (Greiner, #655083), air bubbles were removed and the luminescence was read out on the Tecan Spark 10 (1000 ms attenuation).

### 1.14 CellTrace Violet Proliferation Assay

Transduced T cells (cell pool consisting of transduced GFP+ and non-transduced GFP− cells) were spun down, washed with DPBS and seeded for starvation (4 hours to overnight) in medium without cytokines (advanced RPMI, 10% FBS, 1× Glutamax (assay medium)). Shortly before the staining, the CellTrace Violet dye (ThermoFisher, #C34557) was reconstituted in DMSO according to the manufacturer's instructions (5 mM) and diluted to 5 μM in pre-warmed DPBS. The cells were washed twice with DPBS, counted and the needed amount was spun down and resuspended with diluted CellTrace dye (1 ml for 1 mio cells). The cells were incubated for 20 minutes at 37° C., protected from light, before the reaction was stopped by adding five times the original staining volume of the assay medium to the cells. After an additional 5 minutes of incubation the cells were spun down and the pellet was resuspended in fresh, pre-warmed assay medium. 200.000 cells were seeded in 100 μl in each well of a flat bottom 96-well plate. 100 μl of the wanted compound dilution was added to the wells in duplicates and the plate was incubated for 5-6 days.

For the readout, the cells were resuspended and transferred to a U-bottom 96-well plate for staining. The wells were washed twice with DPBS and then resuspended in staining mastermix (Live/Dead NIR (1:1000, Invitrogen, #L34976), PE anti-CD3ε (1:100, Biolegend, #300408) and Fc_P329G_LALA-AF647 (50 nM). The cells were incubated for 30 minutes at 4° C. and then washed twice with FACS buffer. Then the cells were fixed by adding 100 μl fixation buffer (BD, #554655), incubated for 20 minutes at 4° C., spun down and the supernatant discarded. After resuspending the samples in 150 μl FACS buffer, they were recorded on the BD FACS Fortessa. For the analysis the proliferation in the GFP+ cells was compared with the proliferation of the GFP− cells.

### 1.15 STAT5 Phosphorylation Assay

Transduced T cells (cell pool consisting of transduced GFP+ and non-transduced GFP− cells) were spun down, washed with DPBS and seeded for starvation (4 hours to overnight) in medium without cytokines (advanced RPMI, 10% FBS, 1× Glutamax (assay medium)). The fix buffer I (BD, #557870) was placed at 37° C. and the Perm Buffer III (BD, #558050) was placed at −20° C. The starved T cells were spun down and resuspended in assay medium. 150.000-300.000 cells were seeded in 25 μl in a 96-well V-bottom plate. Cytokines were diluted in assay medium and 50 μl of the dilutions were added to the T cells in duplicates. The plate was incubated for 15 minutes at 37° C., before 75 μl Fix Buffer I were added to the wells. After incubating the plate an additional 30 minutes at 37° C., the plate was spun down (400×g, 3 min) and the supernatant discarded. The cells were resuspended in 100 μl ice cold Perm Buffer III and incubated on ice for 30 minutes. At this stage, the cells were either stained right away or stored at −20° C. for up to two weeks before the pSTAT5 staining.

For the staining, the plate was washed twice with DPBS (400×g, 3 min) before the cells were resuspended in 50 μl diluted AF647 mouse anti-Stat5 (pY694) staining antibody (1:20 in FACS buffer, BD, #562076) and incubated at 4° C. for 1 hour. Then the cells were washed twice with FACS buffer, before the samples were acquired on the BD FACS Fortessa. In order to compare the effect of the cytokine fusion molecules on the non-transduced GFP− cells with the effect on the transduced GFP+ cells, the median fluorescence intensity of the two populations was compared.

### 1.16 Incucyte® Immune Cell Killing Assay

Cancer cell lines (HeLa and MKN45) were in house transduced with the Incucyte® NucLight Red Lentivirus (EF1α, Puro, #4476) and stable cell lines were created under puromycin selection (HeLa NLR, MKN45 NLR). Human pan T cells were isolated, transduced with the desired constructs and in case of the P329G-CD3ε or P329G-Cαβ additionally the endogenous CD3ε or TCRα+B was knocked out. For the assay, HeLa NLR or MKN45 NLR cells were resuspended in RPMI-1640 (Gibco, #42401-018)+2% FCS (Sigma, #F4135-500ML)+1% Glutamax (Gibco, #35050-038) (killing medium) and 10,000 cells seeded in 100 μl in each well of a flat bottom 96-well plate. The plate was incubated for 2-4 hours at 37° C., 5% CO2, until the cells were slightly attached. The primary T cells were counted and adjusted to 10,000 eGFP+ cells/50 μl in the killing medium and added to the attached cancer cells. Adaptor and control antibodies were diluted in killing medium to the desired concentrations and 50 μl were added to the wells. Every condition was pipetted in duplicates. Bubbles were removed from the wells surface and the plates were placed in the Incucyte® S3 machine. Five images of each well were captured every 4 hours over a course of 5 days. The reduction in cancer cell numbers was quantified using an analysis mask counting the amount of red cells.

### Example 2: Preparation of P329G-CAR, P329G-CD3ε and P329G-Cαβ Constructs

DNA sequences encoding the heavy (VH) and light (VL) variable domains of the anti-P329G antibody (VH3VL1), which is specific to the human Fc portion featuring the P329G mutation, were used as single chain variable fragment (scFv) with a (G4S) 4 linker between the variable domains. The amino acid sequence of anti-P329G VH3VL1 scFv is shown in SEQ ID NO:35.

In the P329G chimeric antigen receptor (P329-CAR), the scFv was used in a 4-1BB-CD3ζ CAR format. The scFv was fused to an extracellular stalk (Uniprot P01732 [135-182]) and transmembrane domain of CD8α (TMD) (Uniprot P01732 [183-203]) via a G4S linker, followed by the intracellular co-stimulatory signaling domain of 4-1BB (CD137) (Uniprot Q07011 [214-255]) and the intracellular signaling domain of CD3 ζ (Uniprot P20963 [52-164]) (FIG. 4A). The mature amino acid sequence of the P329G-CAR (VH3VL1) is shown in SEQ ID NO:146.

In an alternative P329G chimeric antigen receptor (P329-CAR), the scFv was used in a CD28-CD3ζ CAR format. The scFv was fused to an extracellular stalk (Uniprot P01732 [135-182]) and transmembrane domain of CD8α (TMD) (Uniprot P01732 [183-203]) via a G4S linker, followed by the intracellular co-stimulatory signaling domain of CD28 (Uniprot P10747 [180-220]) and the intracellular signaling domain of CD3 ζ (Uniprot P20963 [52-164]). The mature amino acid sequence of the P329G-CAR (VH3VL1) is shown in SEQ ID NO:149.

For the P329G-CD3& construct, the scFv was fused to the CD3ε chain (Uniprot P07766 [23-207]) of the TCR complex via a (G₄S)₃ linker (FIG. 4B). The mature amino acid sequence of P329G-(VH3VL1)-CD3& is shown in SEQ ID NO:222.

In the P329G-Cαβ construct, the VH and VL of the anti-P329G domains were directly fused to the constant regions of the TCRα and TCRβ chains (Uniprot P01848 [1-140] and Uniprot P01850 [1-176]), respectively. VH and VL thereby replace the Vα and Vβ domains of the natural TCRαβ chains as indicated in (FIG. 4C). In both chimeric TCR formats, the chains are thought to naturally integrate into the TCR complex (FIG. 4B-C). The mature amino acid sequences of the polypeptides forming the P329G-(VH3VL1)-Cαβ constructs are shown in SEQ ID NO:235 and SEQ ID NO:255.

A graphical representation of an exemplary expression construct including the enhanced green fluorescent protein (eGFP) expression marker is shown in FIG. 1B for the P329G-CAR and in FIG. 1C for the P329G-CD3ε and P329G-Cαβ respectively. The individual protein coding genes are separated by T2A or E2A self-cleaving peptide sequences.

### Example 3: Gene Knockout of Endogenous CD3ε in Jurkat NFAT Cells

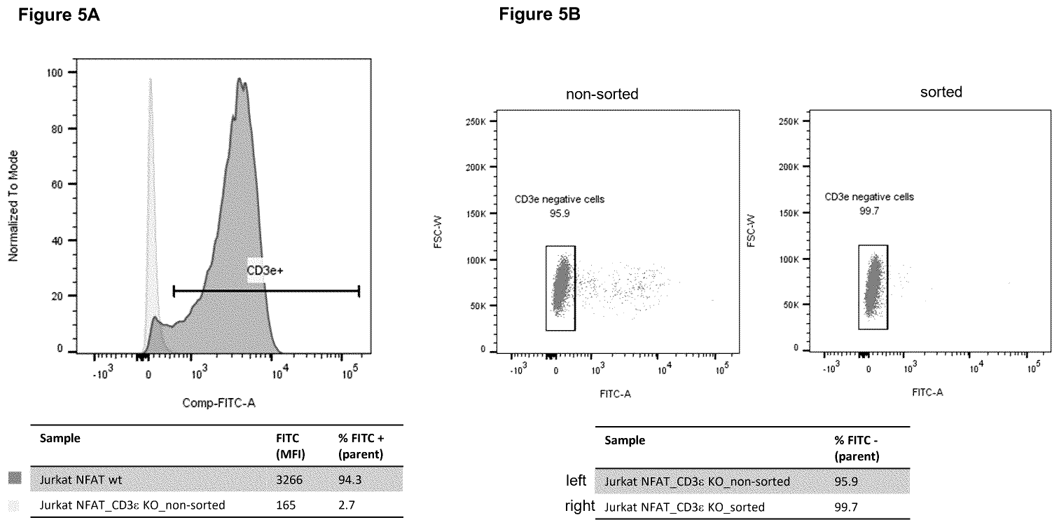

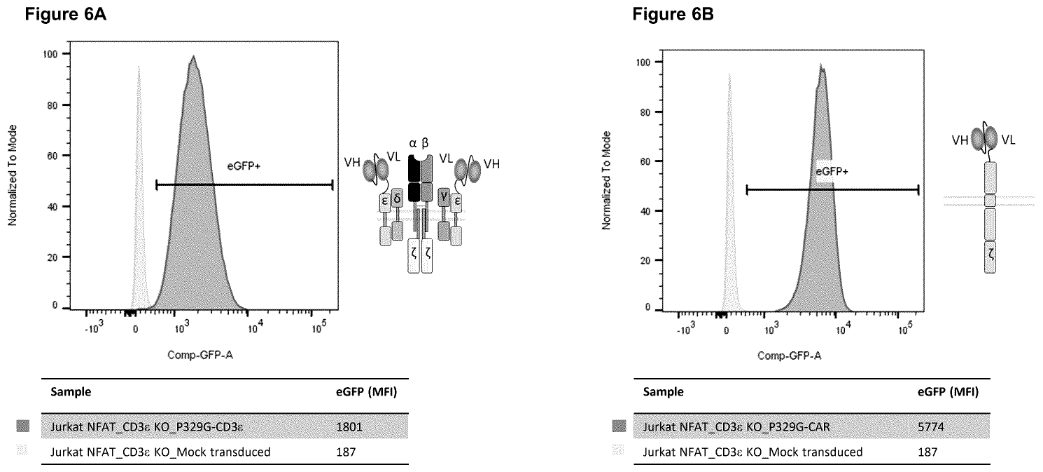

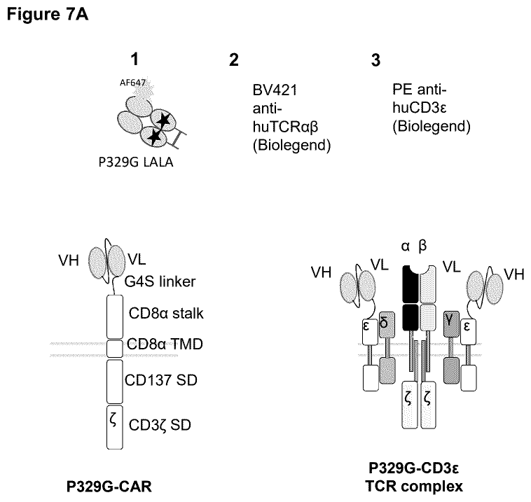

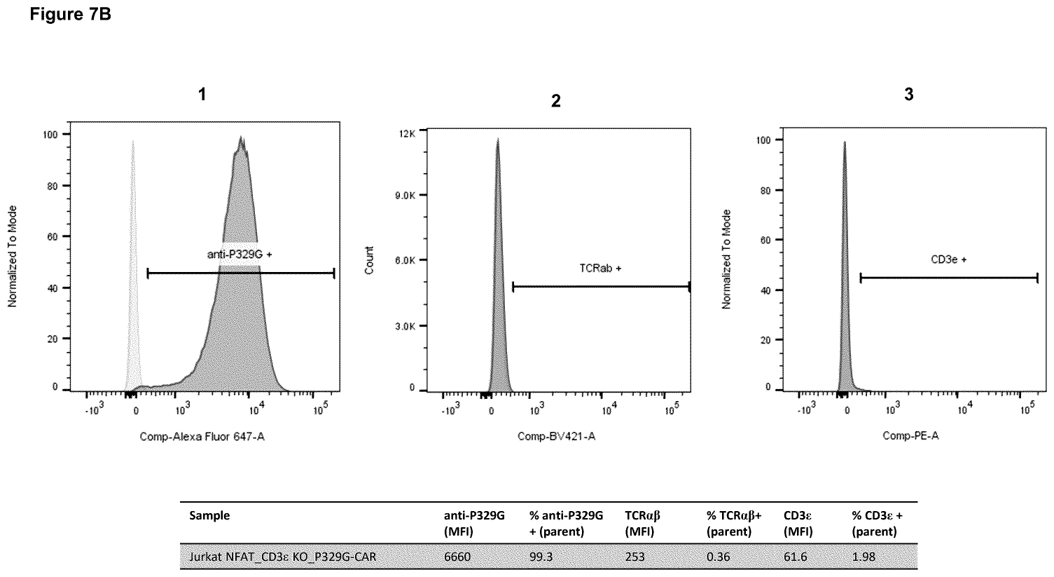

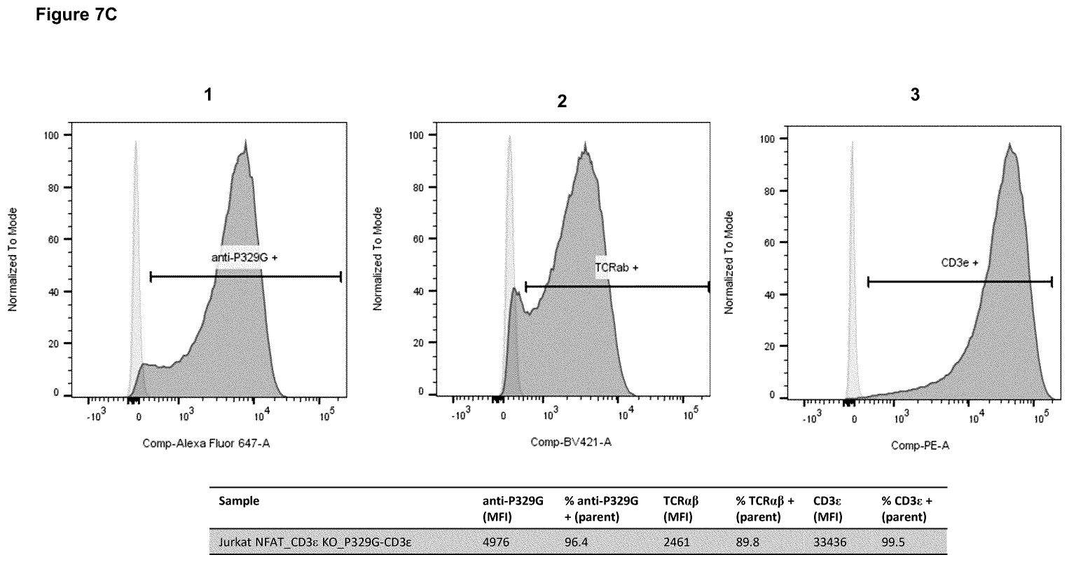

In order to characterize the P329G-CD3ε TCR complex, CD3ε-negative Jurkat NFAT reporter cells were generated by CRISPR/Cas9-mediated gene knockout of the endogenous CD3E gene. The knockout prevents the formation of mixed TCR complexes containing the wild type CD3ε as well as the modified P329G-CD3& chains. RNPs using the sgRNA targeting Exon 7 of CD3E (SEQ ID NO:280) were generated and the knockout was performed as described above. The cells were afterwards resuspended in 1 ml of RPMI-1640, 10% FBS, 1% Glutamax (no antibiotics) and incubated for 3 days (37° C., 5% CO₂, humidified) before flow cytometry analysis to verify the CD3E gene knockout (FIG. 5A). Cells were purified by sorting using the FACSAria™ III gated on CD3ε negative, living cells (as described earlier) and then reanalyzed by flow cytometry (see FIG. 5B). The cells were 99.7% CD38-negative after this procedure and ready to be used for further experiments.

### Example 4: Expression of P329G-CAR or P329G CD3ε or P329G-Cαβ in Jurkat NFAT CD3ε KO or Jurkat TCRαβ KO CD4+ Cells

The P329G-CAR, P329G-CD3ε or P329G-Cαβ receptors were transduced with virus-like particles (VLPs) into Jurkat NFAT CD3ε KO or Jurkat TCRαβ KO CD4+ cells, as described above. Cells were pool sorted for eGFP expression or eGFP and anti-P329G co-expression. Expression of chimeric receptors was assessed and compared by flow cytometry. Transduced Jurkat cells were harvested, washed with DPBS and seeded at 100.000 cells per well in a 96 well U bottom plate. The cells were stained with LIVE/DEAD™ Fixable Near-IR Dead (Invitrogen, #L34976) dye (1:1000 in DPBS) for 20 minutes at 4° C., and washed twice with FACS-buffer (1×DPBS, 2% FBS, 5 mM EDTA pH 8.0, 0.05% NaN3). Then the cells were resuspended in 50 μl FACS buffer with 100 nM fluorescently labeled (Alexa Fluor 647) Fc fragments featuring the previously described P329G LALA mutations (Fc-P329G LALA-AF647). To assess the integration in the endogenous TCR complex, the cells were also stained with anti-CD36 (1:50, −PE, Biolegend, #300408 or 1:50, −APC, Biolegend, #300412) and anti-TCRαβ-BV421 (1:50, Biolegend, #306721) and incubated for 20 minutes at 4° C. After two washing steps the cells were fixed (BD CytoFix, #554655) and analyzed on the FACS.

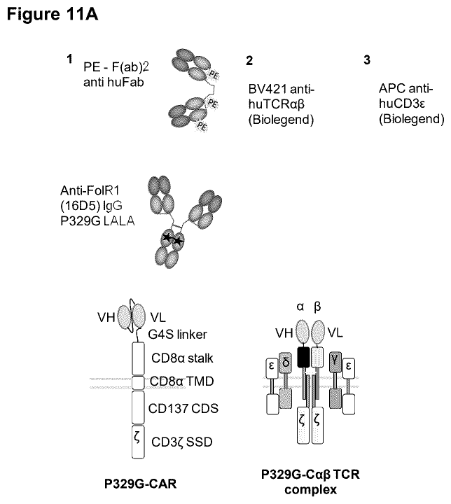

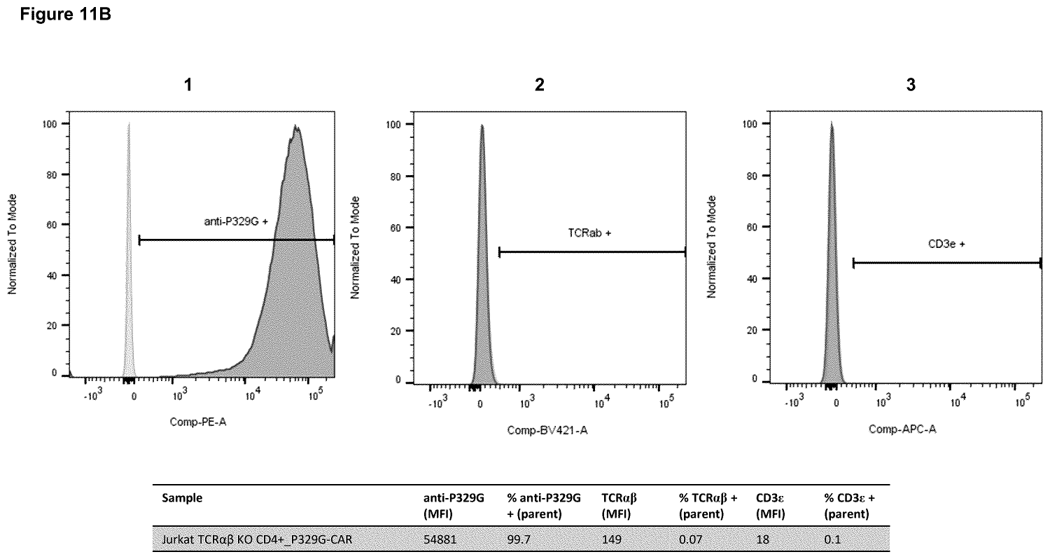

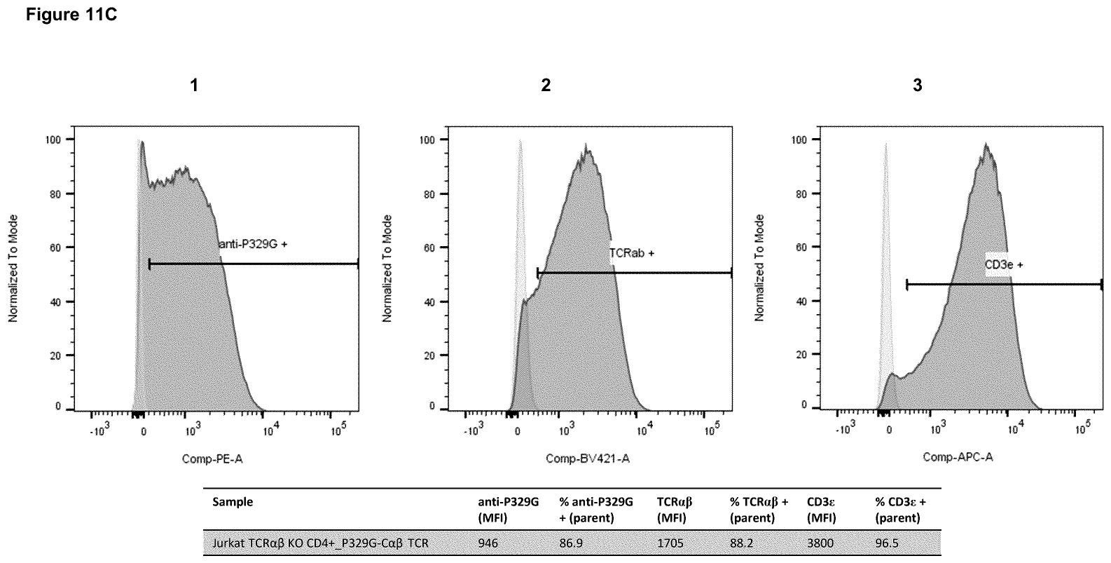

Expression of the transgenic Jurkat TCRαβ KO CD4+ cells expressing the P329G-CAR or P329G-Cαβ constructs. Notably, the eGFP levels of the Jurkat cells transduced with the P329G-Cαβ construct were much lower compared to the eGFP expression of the P329-CAR Jurkat cells. This might be due to the larger size of the construct and also the position of the eGFP gene in the construct (third gene vs, second gene). FIGS. 11B (1) and 12C (1) show the surface expression of the receptors (87-99% positive), while FIGS. 11B (2+3) and 12C (2+3) show the integration of the P329G-Cαβ construct into the endogenous TCR complex, as the CD3ε chains are only detectable after transduction with the chimeric Cαβ construct. In summary, the P329G-CAR, P329G-CD3E and P329G-Cαβ constructs were shown to be expressed on the surface of the Jurkat cells and the Cαβ or CD3& fusion constructs were shown to integrate into the natural TCR complex of the cells.

The functionality of the different anti-P329G receptors was assessed in subsequent Jurkat activation assays.

### Example 5: Specific T Cell Activation in the Presence of Adaptor Antibody Comprising the P329G Mutation

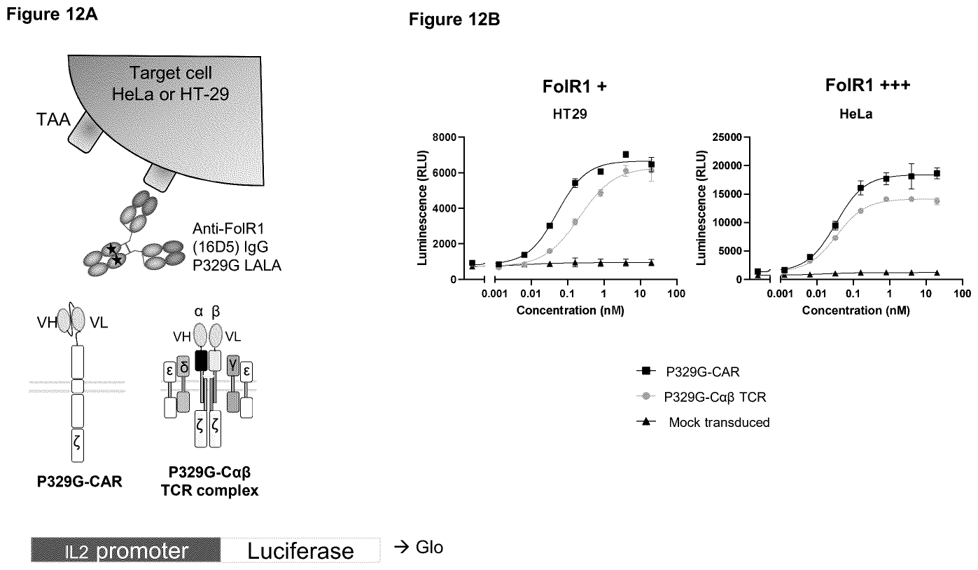

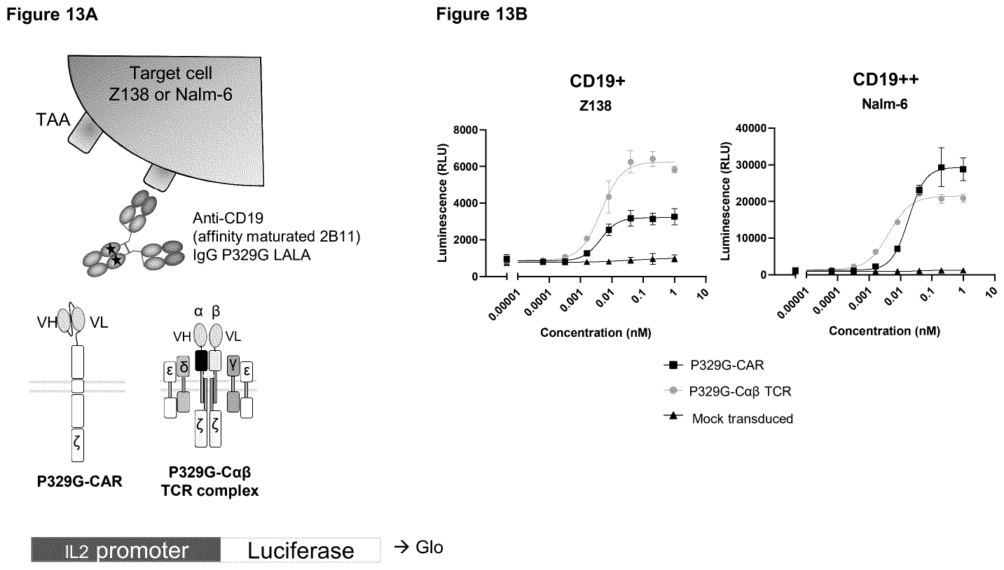

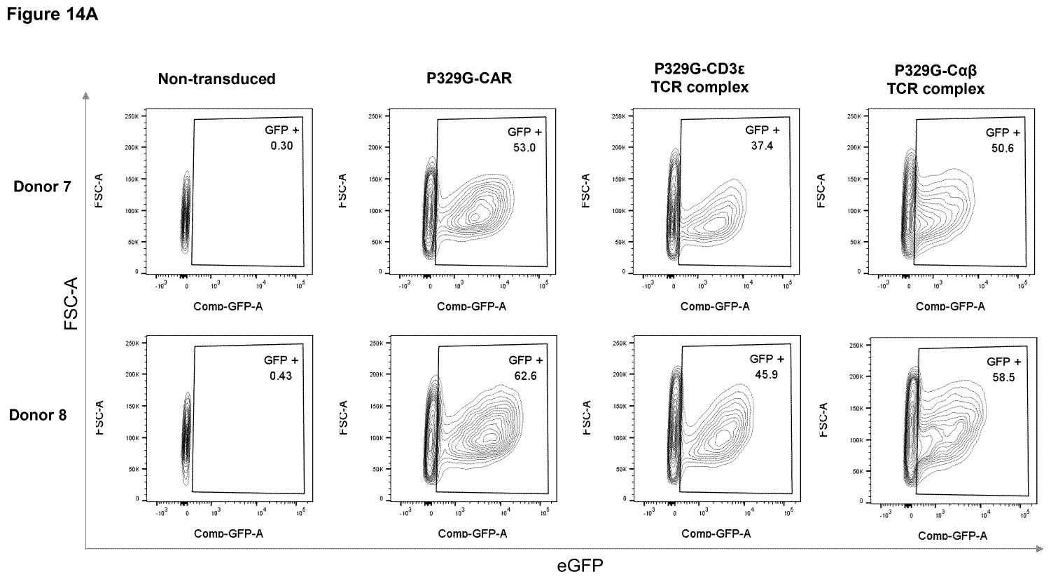

To assess and compare specific T cell activation for T cells expressing P329G receptors having the formats shown in FIG. 4. P329G-CAR, P329G CD3ε or P329-Cαβ transduced Jurkat cells were evaluated for their activation in the presence of FolR1-positive target cells, and anti-FolR1 IgG P329G LALA as targeting adaptor (FIGS. 8A and 12A). The same transgenic Jurkat cell pools were also analysed for their activation in the presence of CD19-positive target cells, and anti-CD19 IgG P329G LALA (FIGS. 9A and 13A). More specifically, transgenic P329G-receptor-positive Jurkat cells were tested with cell lines expressing FolR1 at high (HeLa) or low (HT29) levels. Similarly, cells expressing CD19 at high (Nalm-6) or low (Z138) levels were evaluated. Mock-transduced Jurkat cells (transduced with VLPs lacking a transgene vector) served as a negative control. The Jurkat activation assay was performed as described in detail above.

Dose-dependent and also antigen level-dependent activation of the transgenic Jurkat cells was observed with all tested P329G-specific constructs. In all target antigen-expressing cell lines investigated, the P329G-CD3ε Jurkat cells displayed higher activation compared to cells expressing P329G-CAR. The difference was especially pronounced with cell lines expressing the relevant target antigen at low levels. The mock-transduced control cells displayed were not activated in the presence of anti-FolR1 or anti-CD19 IgG P329G LALA adaptor molecules and any of the target antigen-expressing cell lines investigated (FIGS. 8B, 8B).

The level of activation of T cells expressing the P329G-Cαβ was similar to the level of activation observed for T cells expressing P329G-CAR (FIGS. 12A, 12B and 13B), although the level of surface expression of P329G-Cαβ was much lower than the level of surface expression of P329G-CAR (FIGS. 11B (1) and 11C (1)). In the Z138 model (in which the cells express low levels of CD19), T cells expressing P329G-Cαβ showed higher activation than T cells expressing P329G-CAR (FIG. 13B).

In summary, P329G-CAR, P329-CD3E and P329G-Cαβ constructs were shown to be functional and selective activation by adaptor IgGs comprising the P329G mutation was observed. The P329G-CD38 construct showed superior activation compared to the P329G-CAR in all of the models tested, while the P329-CD3ε construct showed similar activation compared to the P329G-CAR, despite having lower overall expression at the cell surface. The next step was to test and compare the expression and activity of the construct in primary T cells.

### Example 6: Expression of P329G-CAR, P329-CD3ε or P329G-Cαβ in Primary T Cells

Constructs encoding the P329G-CAR, P329G-CD3ε or P329G-Cαβ receptors were transduced with virus-like particles (VLPs) into human Pan T cells of two donors as described above. For cells engineered to express P329G-CD3ε or P329G-Cαβ, the endogenous nucleic acid encoding CD3ε or TCRαβ (respectively) were knocked-out using CRISPR-Cas9, 24 hours after transduction (see above). The sgRNAs were designed in such a way to cut the endogenous CD3E or TRBC1/TRAC loci, but not the chimeric constructs, by removing the Protospacer Adjacent Motif (PAM) and adding several mismatches to the binding site. The CD3ε KO was performed using a sgRNA targeting Exon 7 of huCD3E (SEQ ID NO: 280). The TCRαβ KOs were performed using sgRNA targeting human TRBC1 (SEQ ID NO:281), and sgRNA targeting human TRAC (SEQ ID NO:282).

The expression of the chimeric receptors and gene knockout was assessed and compared by flow cytometry on day 5 after transduction, as follows. Transduced T cells were harvested, washed with DPBS and seeded at 100,000 cells per well in a 96-well U bottom plate. The cells were stained with LIVE/DEAD™ Fixable Near-IR Dead (Invitrogen, #L34976) dye (1:1000 in DPBS) for 20 minutes at 4° C. and then washed twice with FACS-buffer (1×DPBS, 2% FBS, 5 mM EDTA pH 8.0, 0.05% NaN3). Then the cells were resuspended in 50 μl FACS buffer with 100 nM Fc-P329G LALA-AF647 and anti-CD3ε-PE (1:50, Biolegend, #300408) and incubated for 20 minutes at 4° C. After another two washing steps the cells were fixed (BD CytoFix, #554655) and analyzed on the FACS.

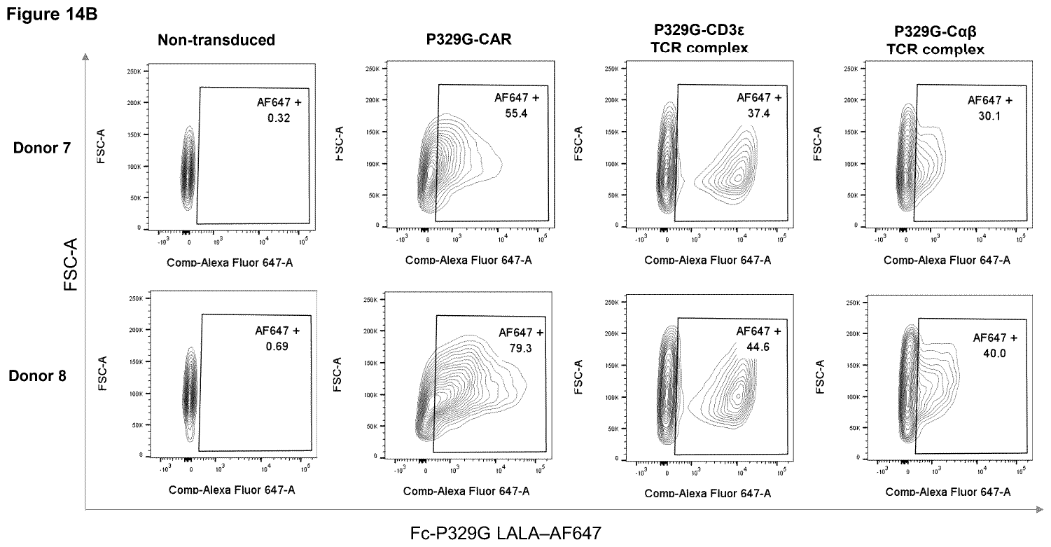

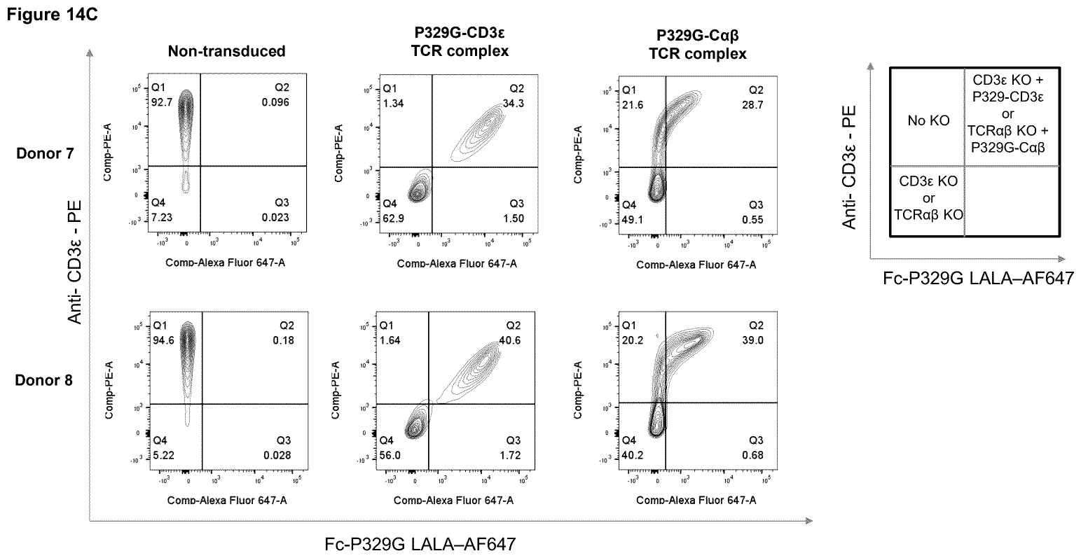

The intracellular eGFP expression is shown in FIG. 14A. For donor 7, 37-53% of the cells were eGFP positive, depending on the construct Donor 8 showed slightly higher eGFP levels following transduction (46-63%). FIG. 14B shows the receptors' surface expression and their ability to bind to Fc-P329G LALA Plotting the CD3 expression and Fc-P329G LALA-AF647 expression (FIG. 14C) revealed that for the P329G-CD3ε, the CD3ε KO was almost complete (only 1.34 (Donor 7) or 1.64 (Donor 8) CD3ε+/Fc-P329G LALA-AF647− cells) and 34-40% of the T cells express the P329G-CD3a TCR complex. For the P329G-Cαβ+TCRαβ−/Fc-P329G LALA-AF647+ population of 29-39% was achieved.

The P329G-CAR and the P329G-CD3ε or P329G-Cαβ constructs were shown to be transduced and expressed on the surface of the primary T cells. To test the functionality of the constructs, they were assessed using an Incucyte® immune cell killing assay.

### Example 7: Expression of P329G-CAR (4-1BB) in CTLL-2 Cells

The P329G-CAR (4-1BB) receptor (SEQ ID NO: 143) was transduced with virus-like particles (VLPs) into CTLL-2 cells as described above. Expression of the CAR was assessed by flow cytometry as follows. Transduced CTLL-2 cells were harvested, washed with DPBS and seeded at 100.000 cells per well in a 96-well U-bottom plate. The cells were stained with LIVE/DEAD™ Fixable Near-IR Dead (Invitrogen, #L34976) dye (1:1000 in DPBS) for 20 minutes at 4° C., and washed twice with FACS buffer (1×DPBS, 2% FBS, 5 mM EDTA pH 8.0, 0.05% NaN3). Then the cells were resuspended in 50 μl FACS buffer with 100 nM fluorescently labeled (Alexa Fluor 647) Fc fragments featuring the previously described P329G LALA mutations (Fc_P329G_LALA-AF647) and incubated for 20 minutes at 4° C. After two additional washing steps, the cells were fixed (BD CytoFix, #554655) and analyzed on the BD FACS Fortessa.

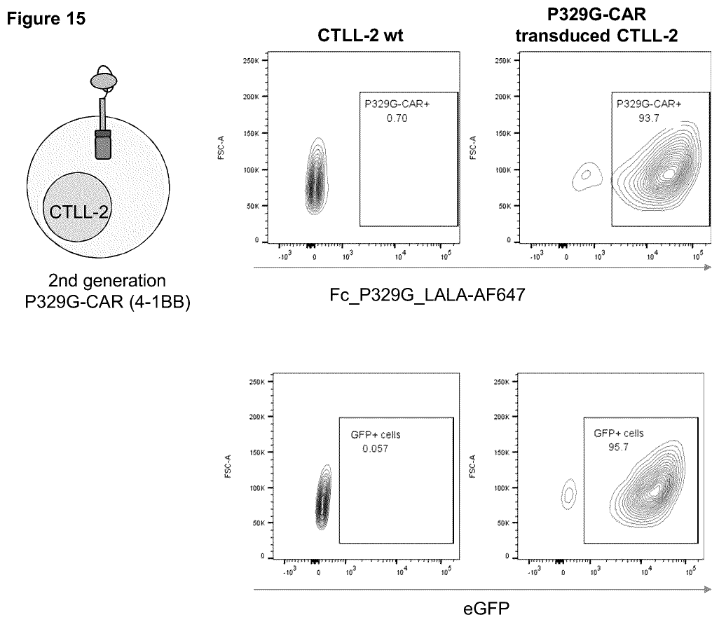

Intracellular eGFP expression of the transduced CTLL-2 cells expressing the P329G-CAR (FIG. 15) show that around 95% of the cells have integrated the construct of interest in the genome. FIG. 15 also shows the surface expression of the receptors (˜94% positive). The IL-2 dependent, transduced CTLL-2 cells were used for a CellTiter Glo viability assay to assess the proliferation upon cytokine stimulation.

### Example 8: CellTiter Glo Viability Assay to Assess Proliferation of P329G-CAR (4-1BB) CTLL-2 Cells

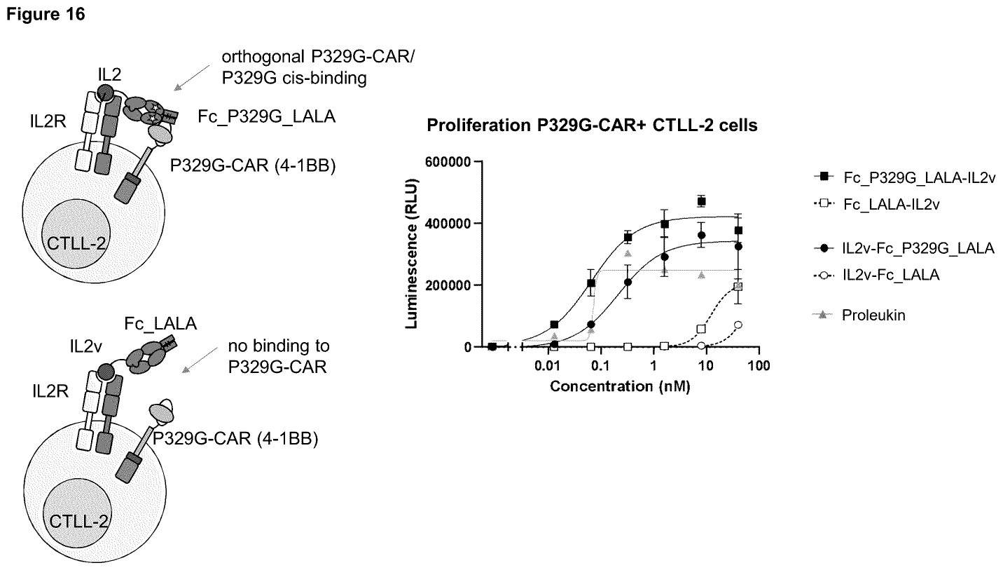

The CellTiter Glo assay was performed with the P329G-CAR (4-1BB) CTLL-2 cells as described in Example 7. A dilution series of Fc_P329G_LALA-IL2v (SEQ ID NOs: 42, 43). Fc_LALA-IL2v (SEQ ID NOs: 42, 45), IL2v-Fc_P329G_LALA (SEQ ID NOs: 42, 47), IL2v-Fc_LALA (SEQ ID NOs: 42, 49) and Proleukin (40 nM, 1:5) was used to check the proliferation of the IL-2 dependent cell line. As shown in FIG. 16, the cells incubated with Fc_P329G_LALA-IL2v or IL2v-Fc_P329G_LALA showed dose dependent proliferation, similar to the Proleukin control. The control molecules without the P329G mutation showed only proliferation at the highest concentrations. The C-terminal IL2v fusion showed higher overall activation compared to the N-terminal IL2v fusion.

### Example 9: Expression of P329G-CAR, P329G-Tag, P329G-CD3ε or P329G-Cαβ in Primary T Cells

The P329G-CAR (SEQ ID NOs: 150), P329G-tags (SEQ ID NOs: 64, 68, 72), P329G-CD3ε (SEQ ID NO: 223) or P329G-Cαβ (SEQ ID NOs: 236, 256) receptors were transduced with virus-like particles (VLPs) into human Pan T cells as described above. For the P329G-CD3ε or P329G-Cαβ constructs the endogenous CD3ε or TCRαβ were knocked out using CRISPR-Cas9 24 hours after transduction (see above). In the transgenes encoding the chimeric receptors, the Protospacer Adjacent Motif (PAM) was removed and several mismatches to the sgRNAs were inserted to avoid being cut by the sgRNAs. The CD3ε KO was performed using a sgRNA targeting Exon 7 of huCD3E (SEQ ID NO:280). The TCRαβ KOs were performed using the following sgRNAs: huTRBC1: SEQ ID NO:281; huTRAC: SEQ ID NO: 282.

The expression of the chimeric receptors and gene knockout was assessed and compared by flow cytometry on day 3-6 after transduction as follows. Transduced T cells were harvested, washed with DPBS and seeded at 100.000 cells per well in a 96-well U-bottom plate. The cells were stained with LIVE/DEAD™ Fixable Near-IR Dead (Invitrogen, #L34976) dye (1:1000 in DPBS) for 20 minutes at 4° C. and then washed twice with FACS buffer (1×DPBS, 2% FBS, 5 mM EDTA pH 8.0, 0.05% NaN3). Then the cells were resuspended in 50 μl FACS buffer with 100 nM Fc_P329G_LALA-AF647 and anti-CD3ε-PE (1:50, Biolegend, #300408) and incubated for 20 minutes at 4° C. After another two washing steps the cells were fixed (BD CytoFix, #554655) and analyzed on the FACS.

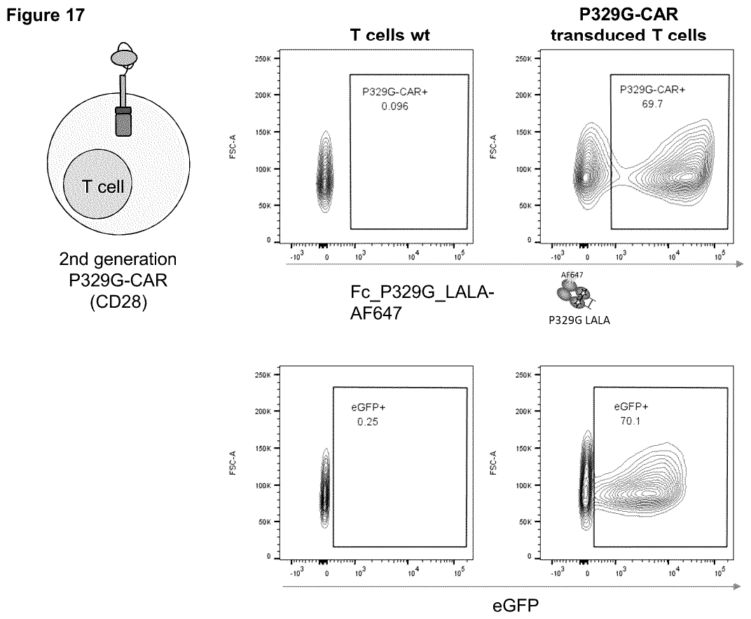

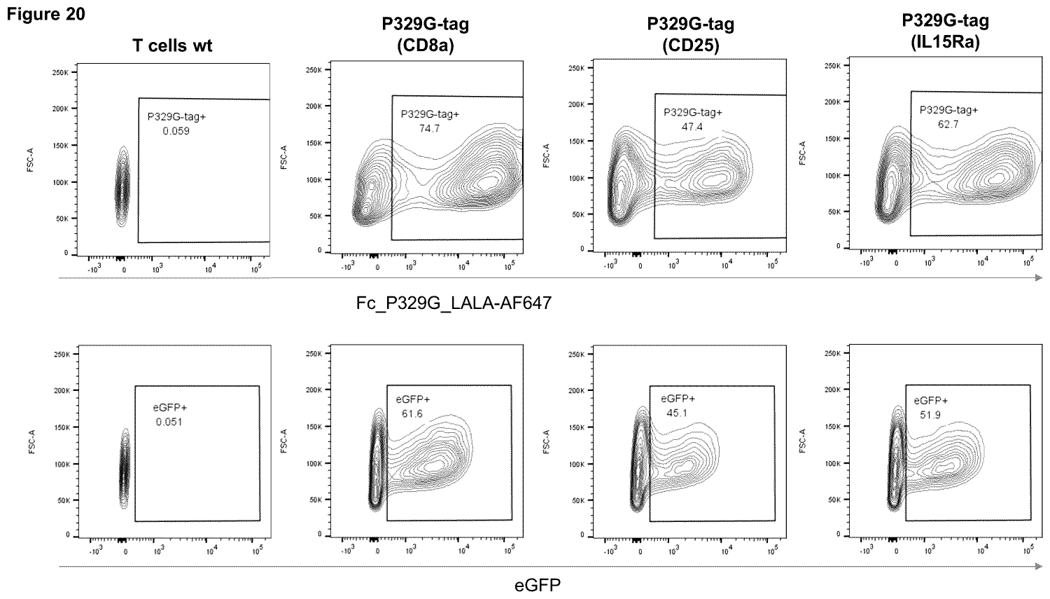

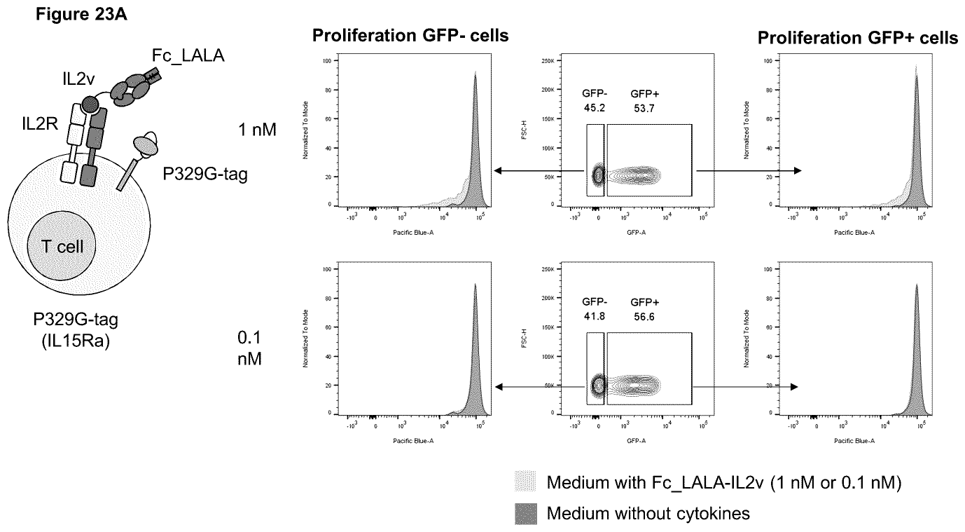

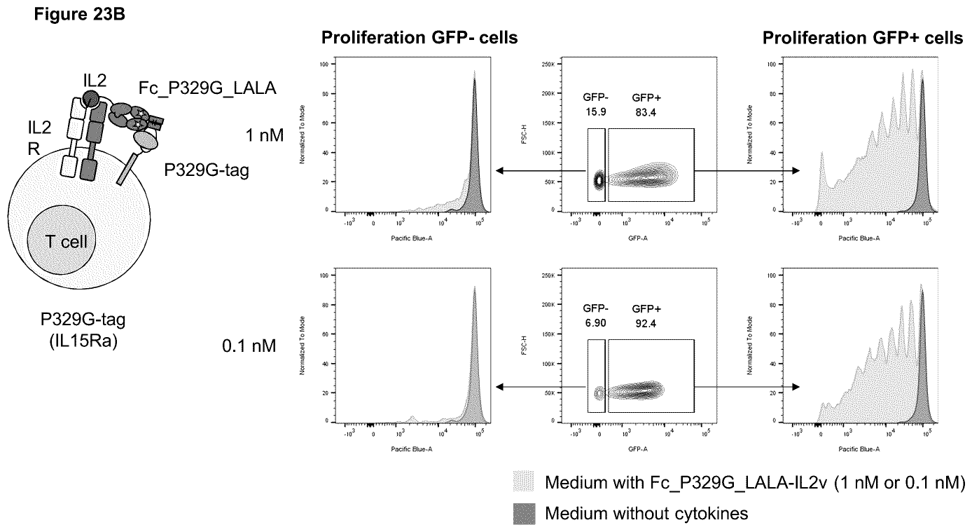

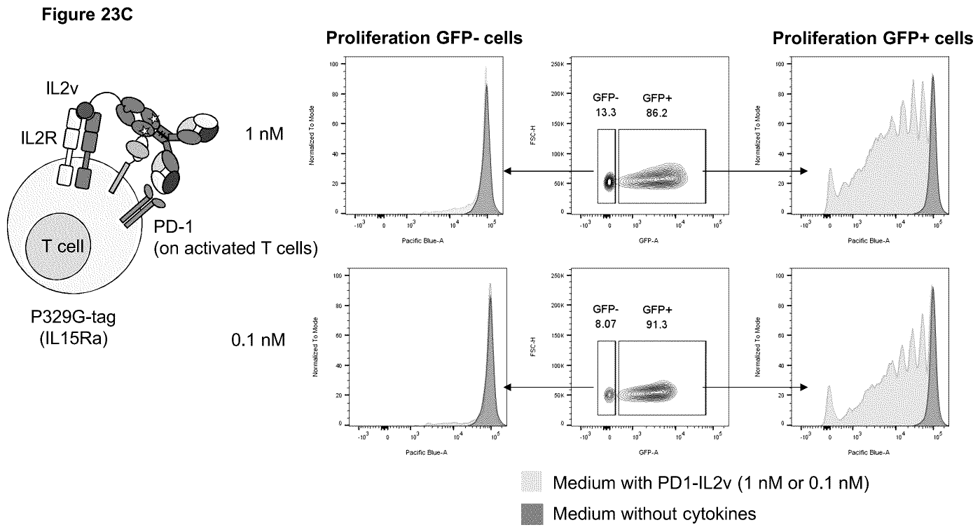

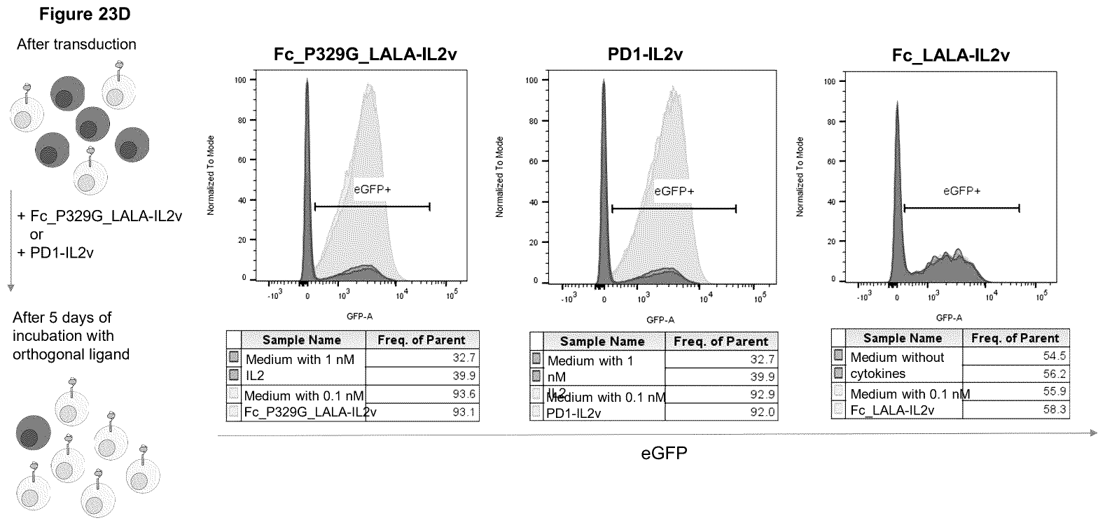

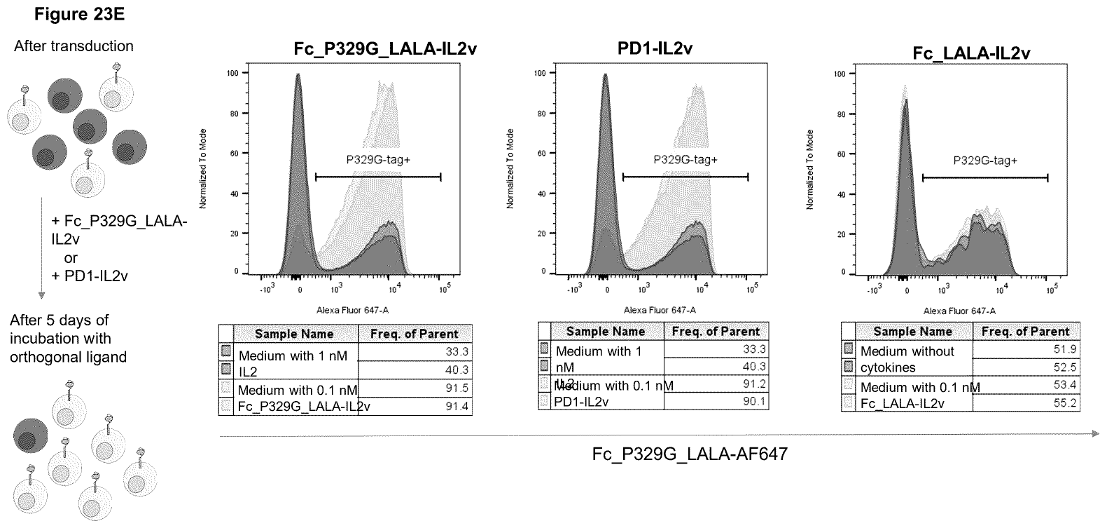

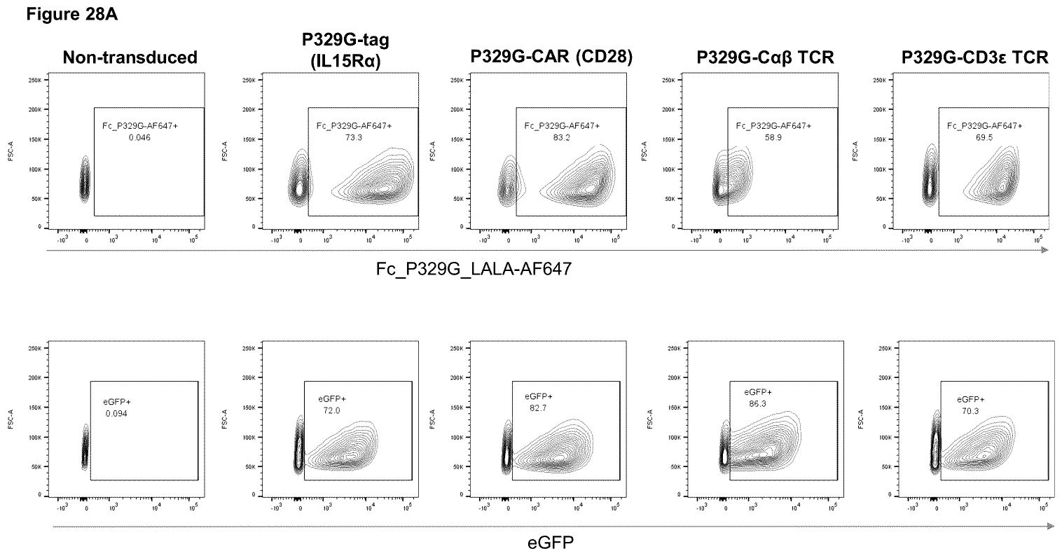

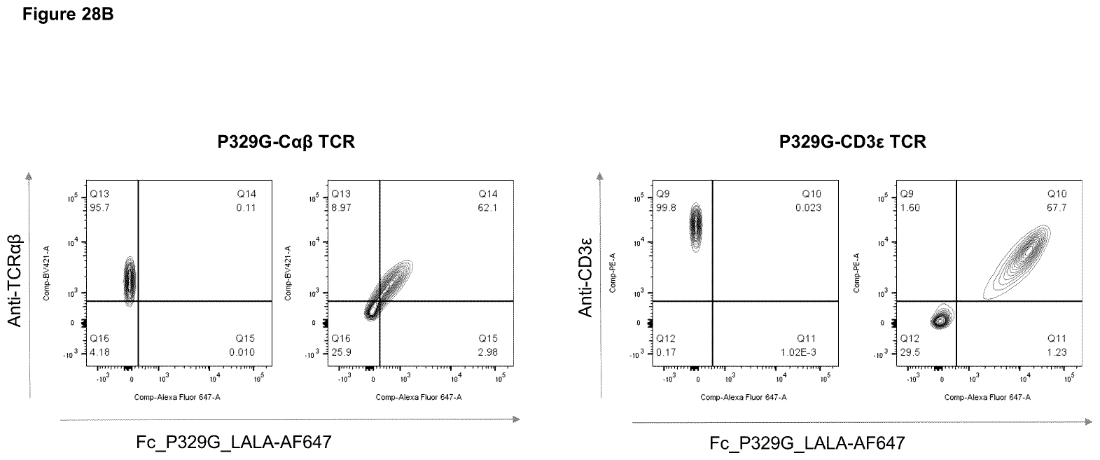

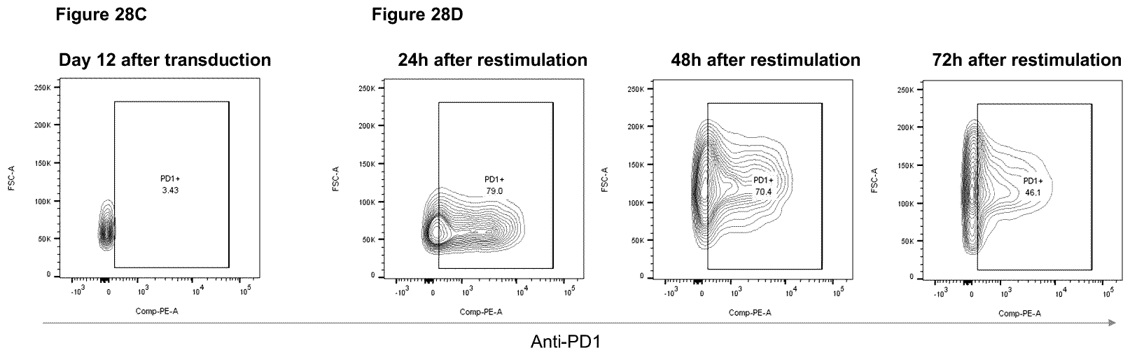

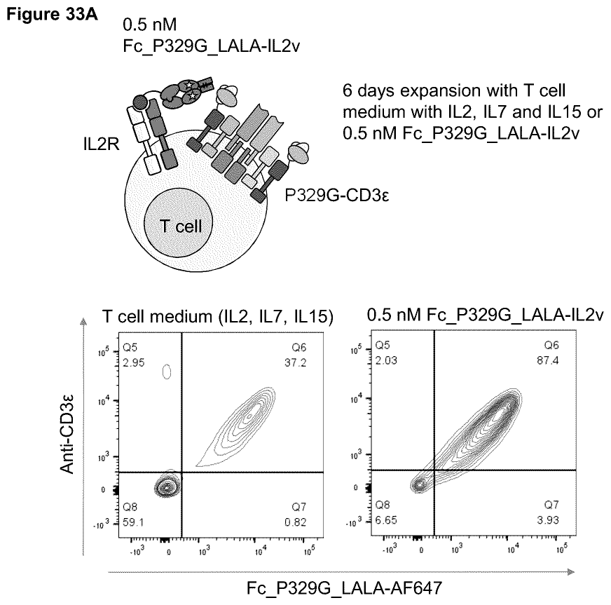

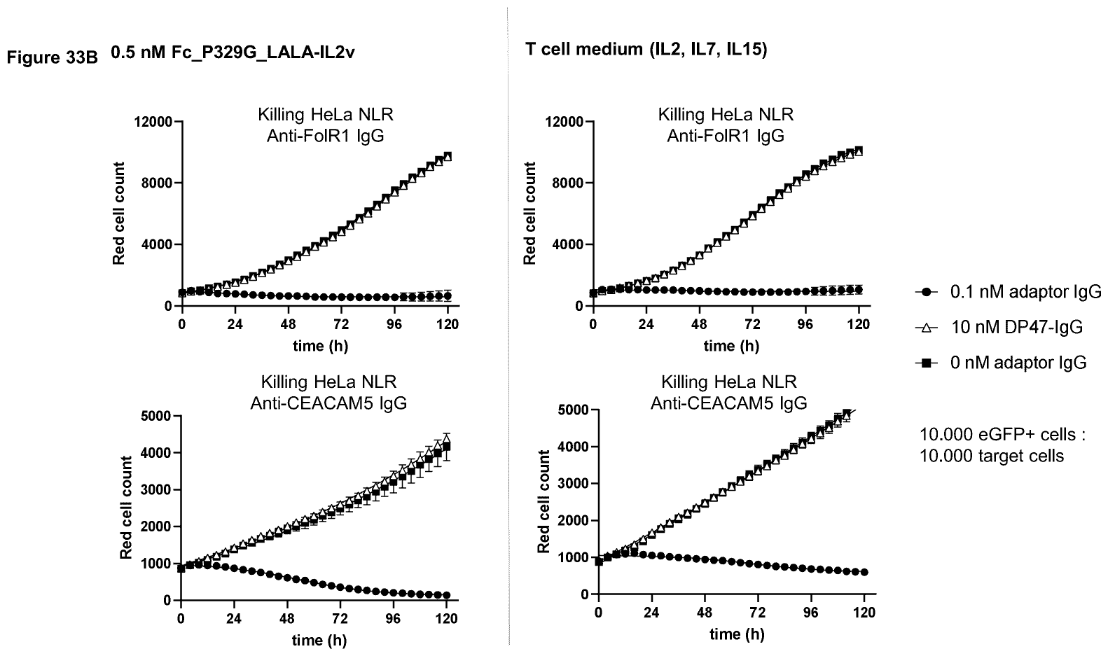

The expression of the constructs for the respective assays is shown in FIG. 17 (P329G-CAR (CD28)), FIG. 20 (P329G-tags), FIG. 23 (P329G-CD3ε), FIG. 28 (comparison of all constructs) and FIG. 33A (P329G-CD3ε for Incucyte killing).

### Example 10: STAT5 Phosphorylation Assay with P329G-CAR (CD28) T Cells

The P329G-CAR comprising the CD28 co-stimulatory domain (SEQ ID NO: 150) was transduced into primary T cells as described above. The transduction efficiency was checked and surface expression of the construct was assessed. ˜70% of the cells were successfully transduced (FIG. 17).

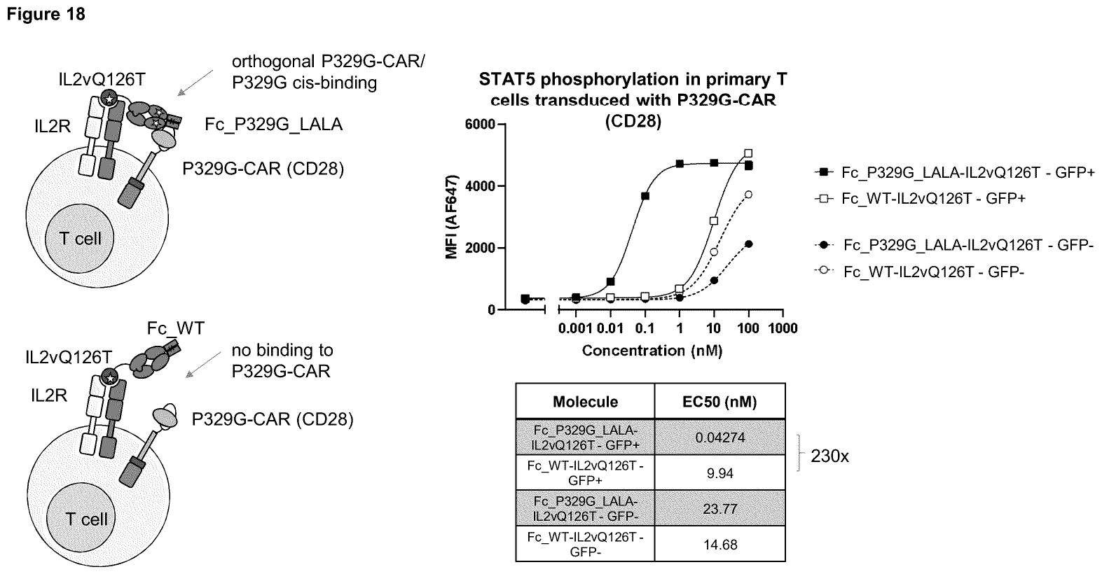

The STAT5 phosphorylation assay was performed on day 7 after transduction with the molecules Fc_P329G_LALA-IL2vQ126T, Fc_WT-IL2vQ126T, PD1-IL2v and PD1-IL2vQ126T. FIG. 18 shows the results of Fc_P329G_LALA-IL2vQ126T vs. Fc_WT-IL2vQ126T on the transduced GFP+ and the non-transduced GFP− cell population. Shown is the median fluorescence intensity (MFI) of the AF647 anti-Stat5 (pY694) antibody. The EC50 of Fc_P329G_LALA-IL2vQ126T on GFP+ (therefore P329G-CAR+) cells in this assay was 0.043 nM. The same molecule on the GFP-population and the control molecule Fc_WT-IL2vQ126T on GFP+/GFP− cells had a much higher EC50 (˜10-24 nM), highlighting the cis-targeting of the Fc_P329G_LALA-IL2vQ126T to the P329G-CAR on primary T cells.

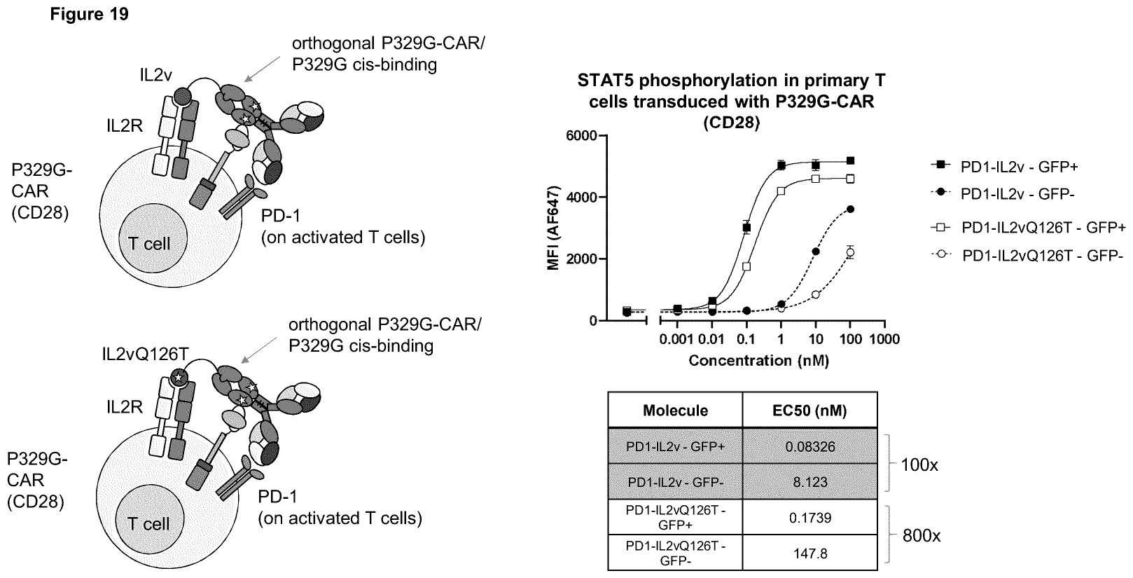

FIG. 19 shows the data obtained with PD1-IL2v vs PD1-IL2vQ126T on the transduced GFP+ and the non-transduced GFP-cell population. Also here, a preferential targeting to the GFP+P329G-CAR T cells was observed compared to the GFP-cells in the same cell pool. This effect can be regarded as independent of the PD1 expression, as PD1 is expected to be equally expressed on GFP+ and GFP− cells. The attenuated IL2vQ126T variant increases the cis-targeting window from ˜100-fold to ˜800-fold in this assay, by decreasing the P329G independent binding to the IL2 receptor.

### Example 11: STAT5 Phosphorylation and Selective Proliferation with P329G-Tag T Cells

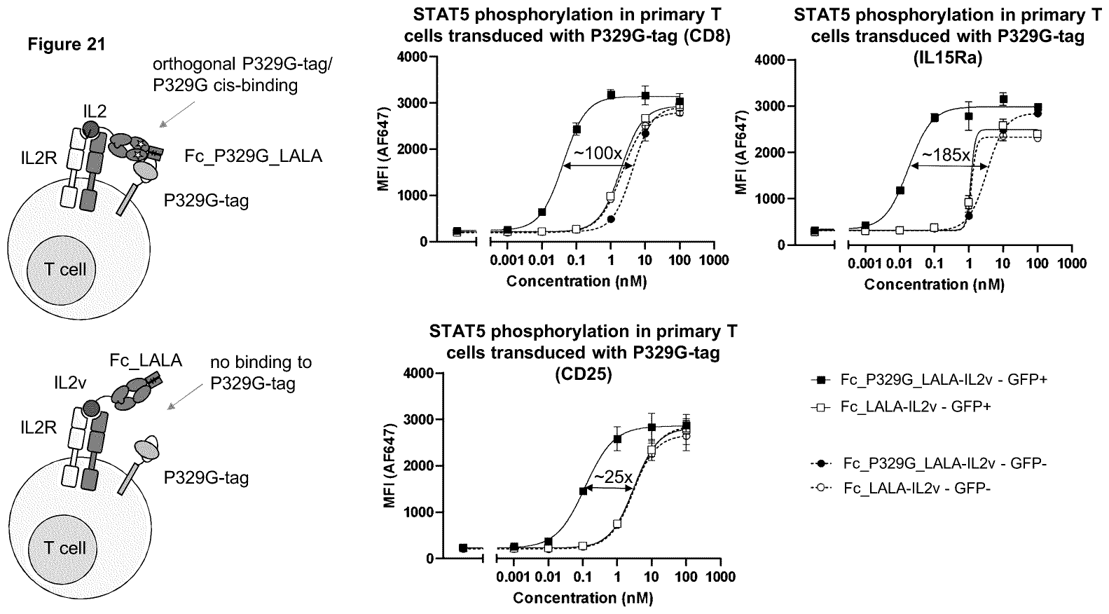

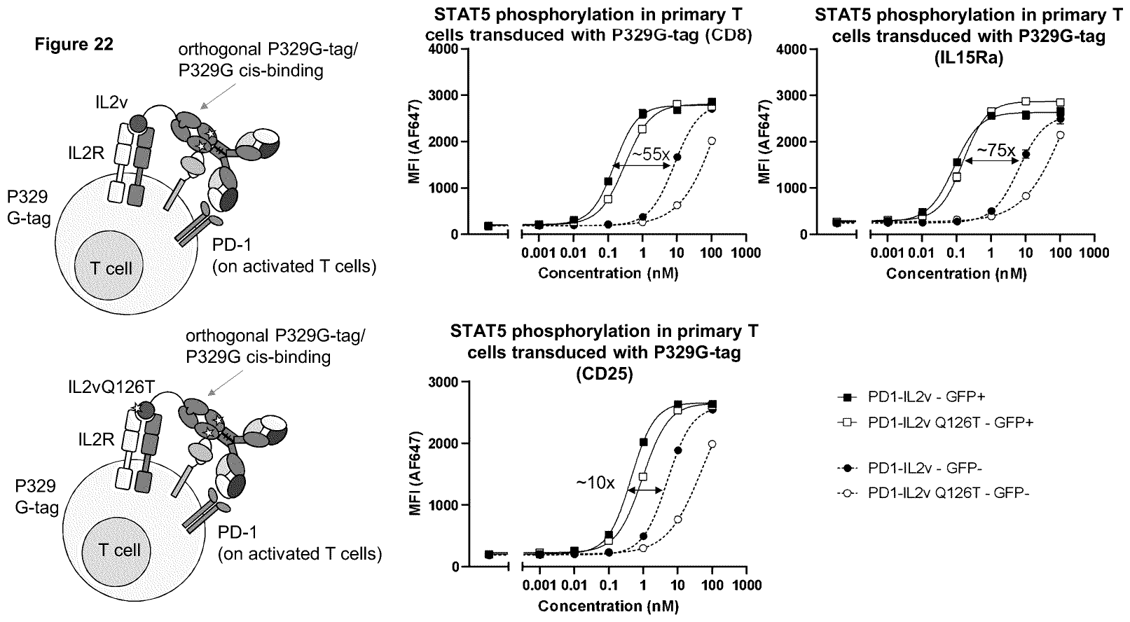

Three different non-signaling P329G-tags designed with CD8α, IL2Rα or IL15Rα membrane-anchors (SEQ ID NOs: 64, 68, and 72) were transduced into primary T cells as described above. The transduction efficiency was checked on day 4 after transduction and surface expression of the construct was assessed. Measured on the GFP expression, ˜45-62% of the cells were successfully transduced (FIG. 20). The selective cytokine targeting to those P329G-tags was assessed on day 15 after transduction by a STAT5 phosphorylation assay with Fc_P329G_LALA-IL2v, Fc_LALA-IL2v, PD1-IL2v and PD1-IL2vQ126T (100 nM, 1:10). FIG. 21 shows the results of Fc_P329G_LALA-IL2v vs. Fc_LALA-IL2v on the transduced GFP+ and the non-transduced GFP− cell population. Shown is the median fluorescence intensity (MFI) of the AF647 anti-Stat5 (pY694) antibody. The best cis-targeting effect (185-fold difference of GFP− vs. GFP+ cells) was observed with the IL15Rα based P329G-tag. FIG. 22 depicts the results with PD1-IL2v vs PD1-IL2vQ126T and also here the best cis-targeting was observed with the IL15Rα P329G-tag (75-fold with PD1-IL2v). Similar to the previous experiment, also here an increase in the cis-targeting window with the IL2vQ126T variant was observed.

The cis-targeting to the P329G-tag (IL15Rα) T cells was also assessed in a CellTrace Violet proliferation assay. The cells were used on day 12 after transduction and the assay was performed as described above. The results of the staining after the 5 day proliferation are shown in FIG. 23. FIG. 23A depicts the proliferation of the GFP+ or GFP-population observed with 1 nM or 0.1 nM of Fc_LALA-IL2v. With 1 nM Fc_LALA-IL2v slight proliferation of the GFP+ and GFP− cell population was observed. With 0.1 nM Fc_LALA-IL2v the proliferation profile resembles the control cells (dark gray), which were incubated without any cytokines. By adding 1 nM or 0.1 nM of Fc_P329G_LALA-IL2v to the wells (FIG. 23B), strong proliferation of the GFP+ cells (cells expressing the P329G-tag) was observed, but only slight proliferation of the GFP-population (non transduced cells) in the same well. This was also seen in the increase of overall GFP+ cells from ˜54-57% in the population incubated with Fc_LALA-IL2v (FIG. 23A) to 83-92% in the population incubated with Fc_P329G_LALA-IL2v (FIG. 23B). The same effect was observed in the cells incubated with PD1-IL2v (1 nM or 0.1 nM) (FIG. 23C). The shift of the transduced population towards GFP+/P329G-tag+ cells after incubation with Fc_P329G_LALA-IL2v is also highlighted again in FIGS. 23D (eGFP expression) and 12E (Fc_P329G_LALA-AF647 staining). Compared to culturing the cells with 1 nM IL2 or 0.1 nM Fc_LALA-IL2v, expansion of the cell pool with 0.1 nM Fc_P329G_LALA-IL2v or PD1-IL2v leads to a >90% transduced population within 5 days.

### Example 12: STAT5 Phosphorylation and Selective Proliferation with P329G-CD3& T Cells

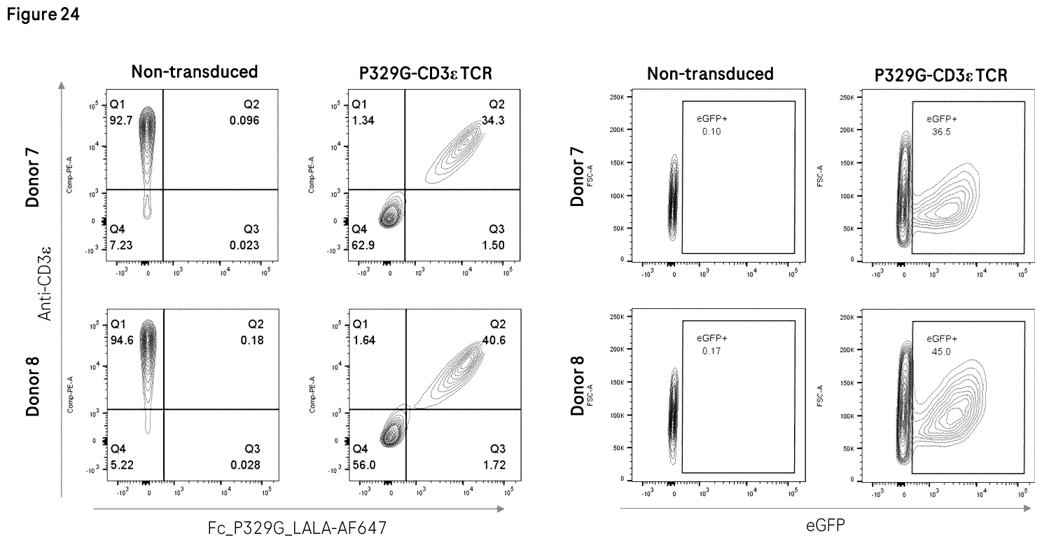

The P329G-CD3ε (SEQ ID NO:223) was transduced into primary T cells and the endogenous CD3ε was knocked out as described above. The transduction/knockout efficiency and surface expression of the construct was assessed by staining with PE anti-CD3ε (1:100, Biolegend, #300408) and Fc_P329G_LALA-AF647 (FIG. 24). 34% (donor 7) or 40% (donor 8) of the cells were expressing the construct of interest on the surface and had a knockout of the endogenous CD3ε (Q2 of the contour plot). Only ˜1-2% were still expressing the endogenous CD3ε (Q1 of the contour plot).

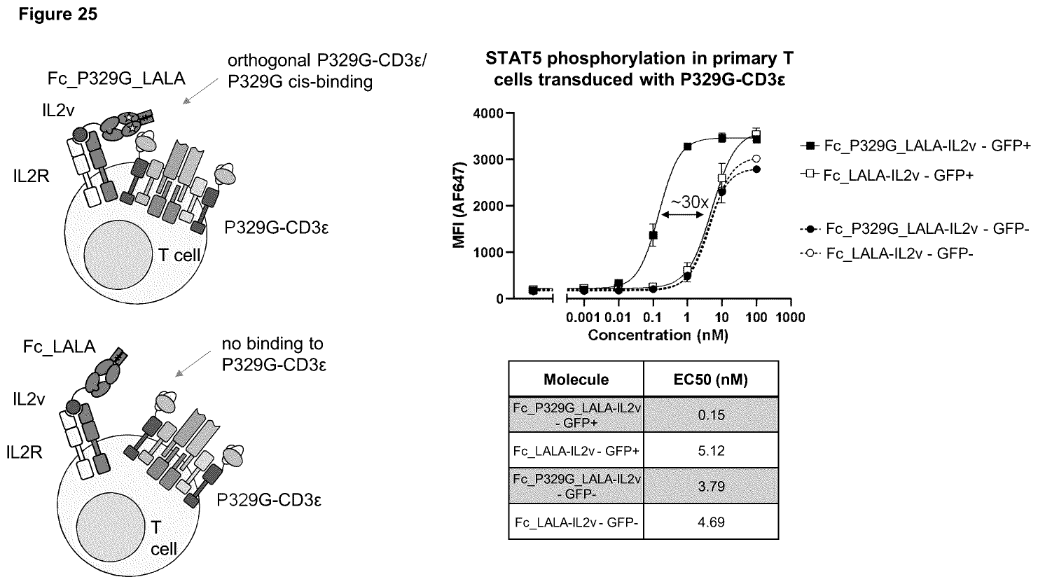

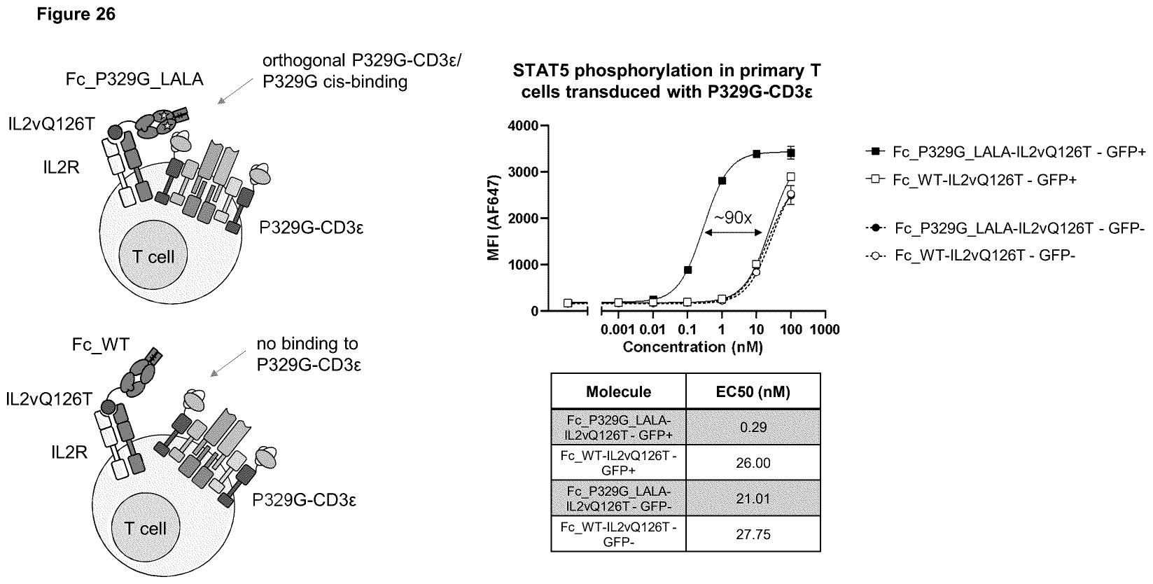

The selective cytokine targeting to P329G-CD3& T cells (donor 8) was assessed on day 13 after transduction by STAT5 phosphorylation assay with the molecules Fc_P329G_LALA-IL2v, Fc_LALA-IL2v, Fc_P329G_LALA-IL2vQ126T and Fc_WT-IL2vQ126T. FIG. 25 shows the results observed with Fc_P329G_LALA-IL2v vs. Fc_LALA-IL2v on the transduced GFP+ and the non-transduced GFP− cell population. Shown is the median fluorescence intensity (MFI) of the AF647 anti-Stat5 (pY694) antibody. An approximately 30-fold cis-targeting effect of GFP+vs. GFP− cells was observed in this assay. With Fc_P329G_LALA-IL2vQ126T this window was increased to ˜90-fold (FIG. 26).

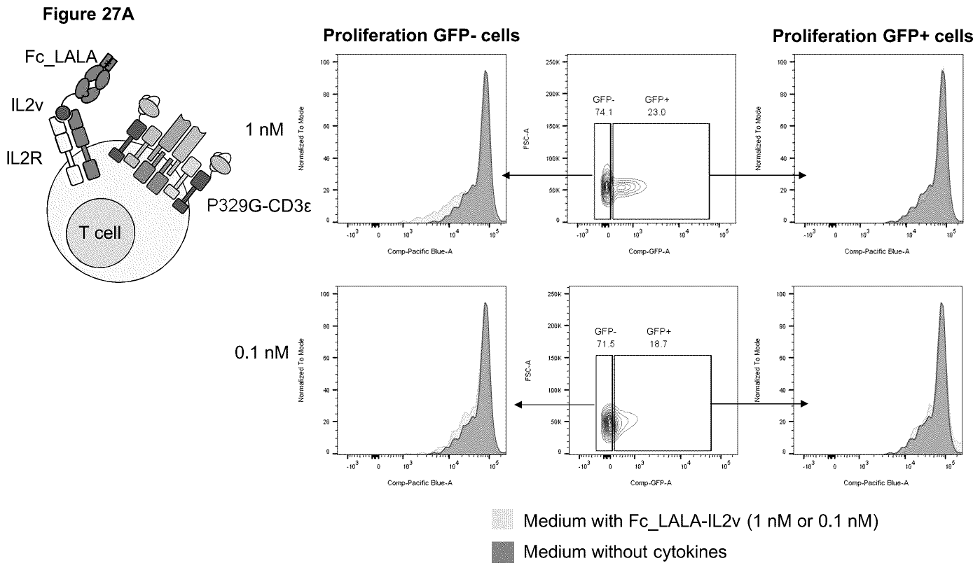

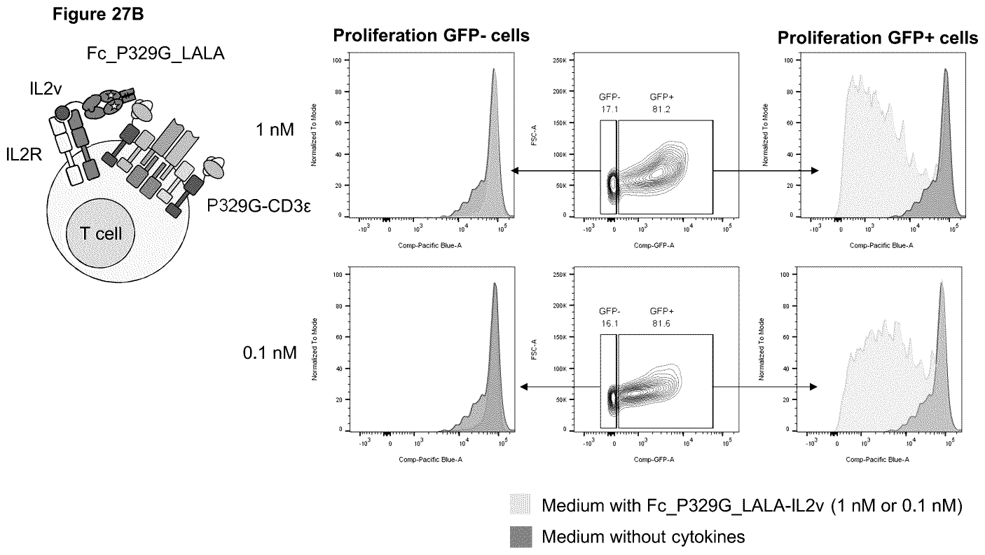

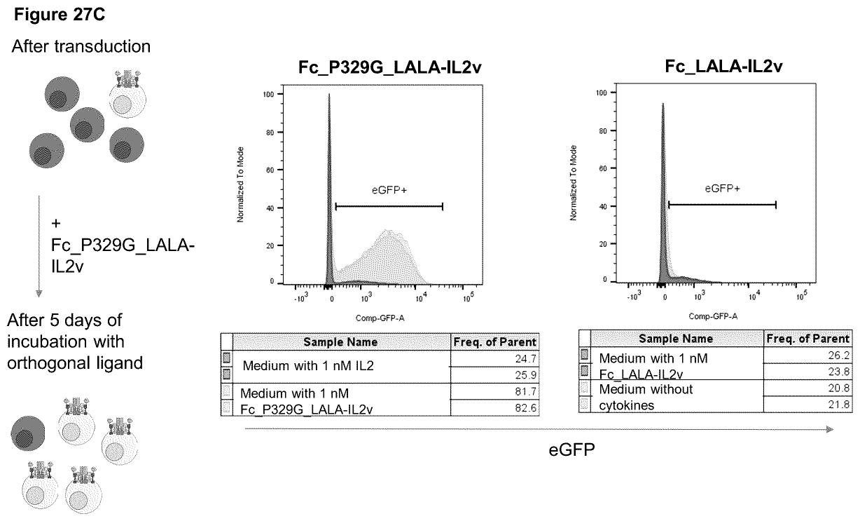

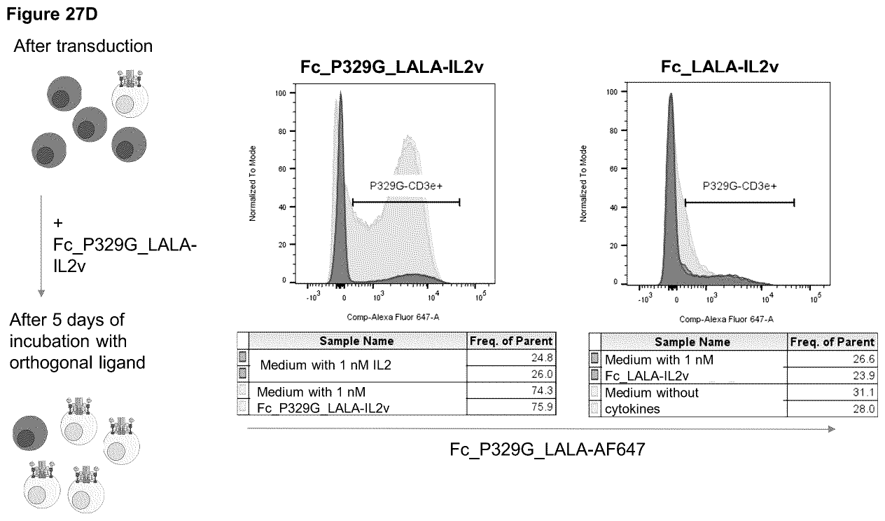

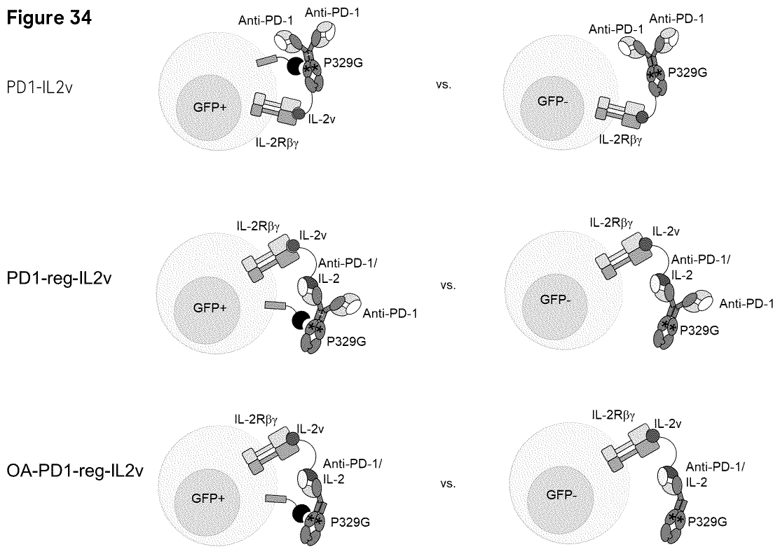

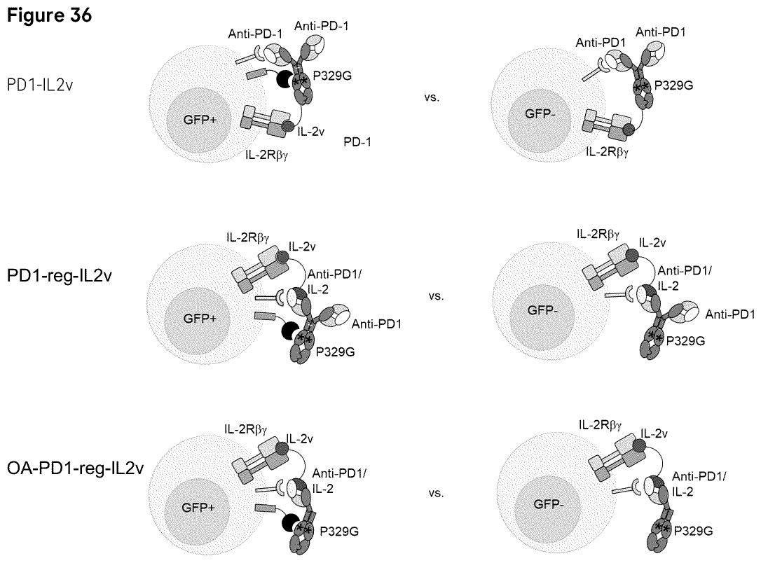

The cis-targeting to the P329G-CD3ε T cells (donor 7) was also assessed in a CellTrace Violet proliferation assay. The assay was started on day 6 after transduction and performed as described above. After 5 days of incubation the cells were stained and the results are shown in FIG. 27. FIG. 27A depicts the proliferation of the GFP+ or GFP− population observed with 1 nM or 0.1 nM or Fc_LALA-IL2v. The proliferation induced by Fc_LALA-IL2v resembles the control cells (dark gray), which were incubated without any cytokines. Incubating the cells with 1 nM or 0.1 nM Fc_P329G_LALA-IL2v (FIG. 27B) resulted in strong proliferation of the GFP+ cells (cells expressing the P329G-CD3ε), but only slight proliferation of the GFP-population (non-transduced cells). This was also seen in the increase of overall GFP+ cells from ˜19-23% in the population incubated with Fc_LALA-IL2v (FIG. 27A) to 81% in the population incubated with Fc_P329G_LALA-IL2v (FIG. 27B). The shift of the transduced population towards GFP+/P329G-CD3ε+ cells after incubation with Fc_P329G_LALA-IL2v is also highlighted again in FIGS. 27C (eGFP expression) and 16D (Fc_P329G_LALA-AF647 staining). Compared to culturing the cells with 1 nM IL2 or 1 nM Fc_LALA-IL2v, expansion of the cell pool with 1 nM Fc_P329G_LALA-IL2v leads to a ˜82% eGFP+ population within 5 days.

### Example 13: STAT5 Phosphorylation with P329G-CAR (CD28), P329G-Tag (IL15Rα), P329G-CD3ε or P329G-Cαβ T Cells

In order to compare the different approaches with respect to P329G-mediated cis-targeting, cells of one donor were transduced to express a P329G-CAR (SEQ ID NO: 150), a P329G-tag (SEQ ID NO:72), P329G-CD3ε (SEQ ID NOs: 223) or P329G-Cαβ (SEQ ID NOs: 236, 256). The transduction and knockout efficiency was checked and surface expression of the construct was assessed. 70-86% of the cells were eGFP positive and shown to express the constructs on the cell surface (Fc_P329G_LALA-AF647 staining) (FIG. 28A). Staining with anti-TCRαβ (1:100, Biolegend, #306721) or anti-CD38 (1:100, Biolegend, #300408) and Fc_P329G_LALA-AF647 revealed that 62% or 68% of the cells were expressing the P329G-Cαβ or P329G-CD3ε construct (FIG. 28B). FIG. 28C shows the PD-1 expression of the P329G-tag (IL15Rα) T cells after transduction and 12 days expansion. The pSTAT5 assays described in the following were performed with those PD-1 low cells. FIG. 28D shows the upregulation of PD-1 expression of the P329G-tag (IL15Rα) T cell pool one to three days after restimulation with ImmunoCult™ Human CD3/CD28/CD2 T Cell Activator (StemCell, #10990), confirming that PD1 expression is only transiently expressed upon T cell activation.

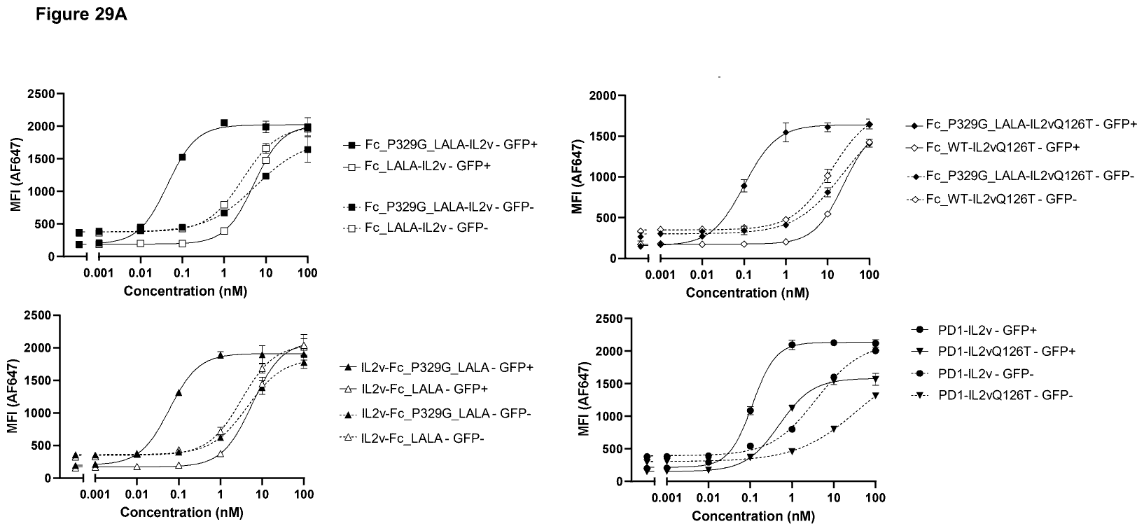

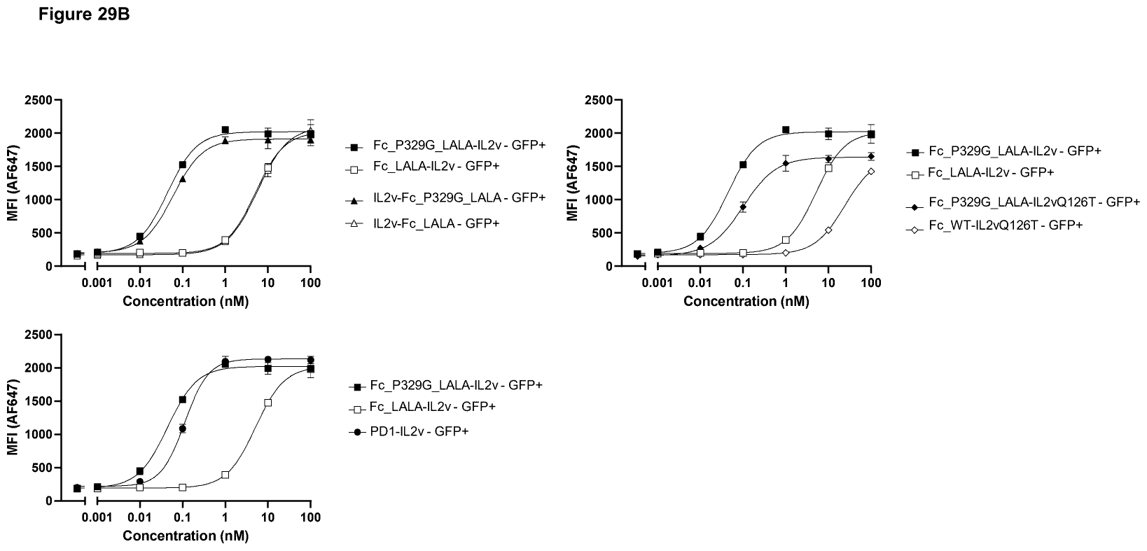

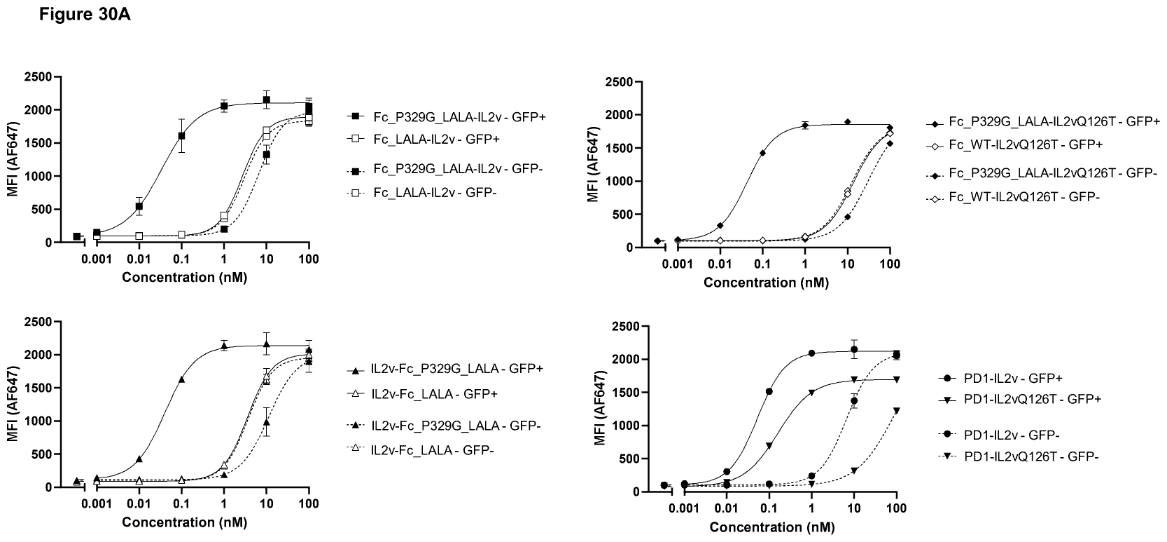

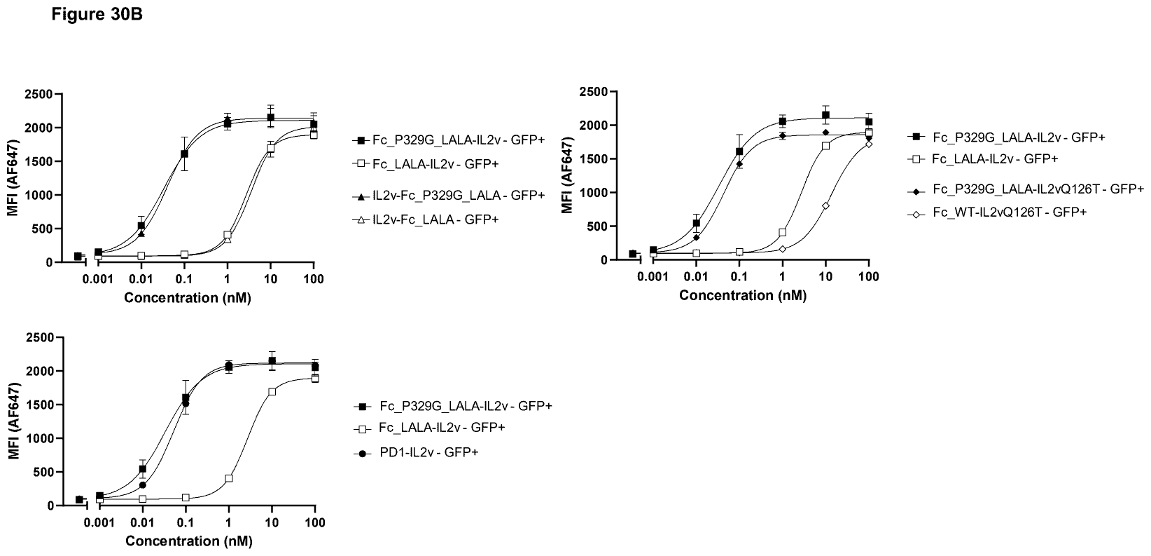

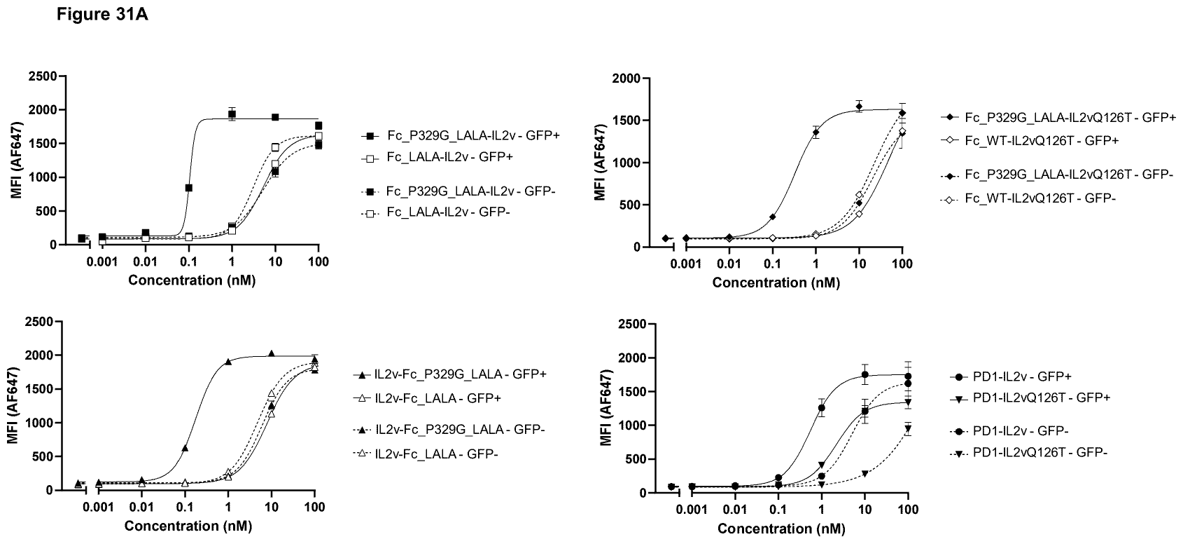

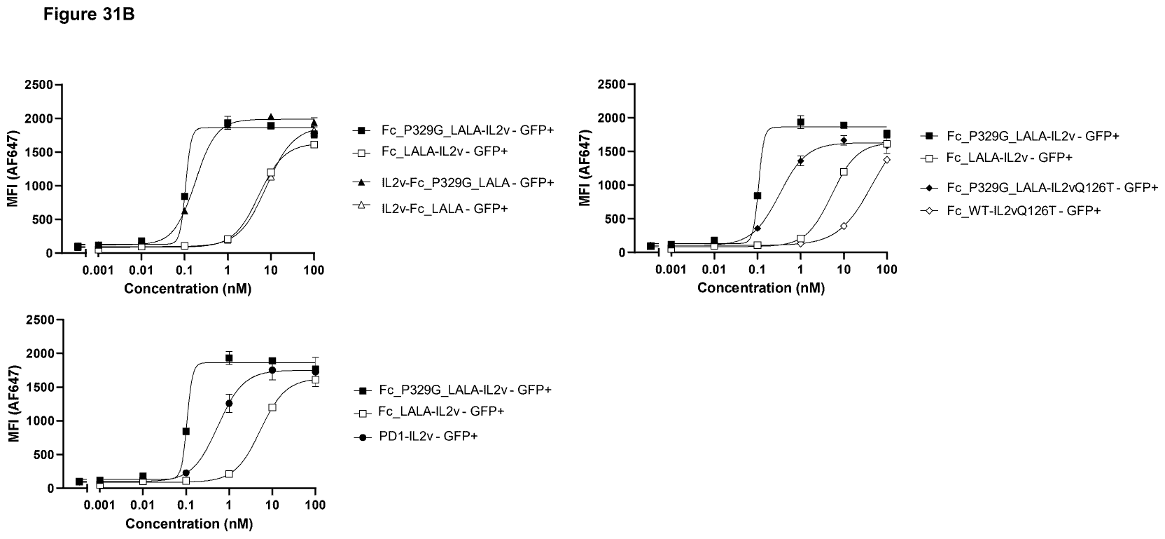

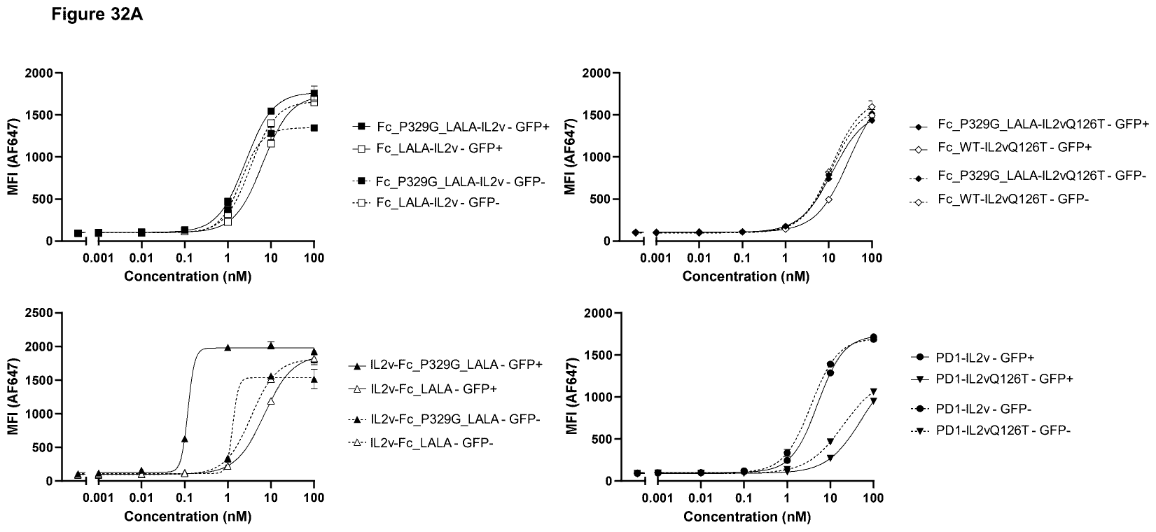

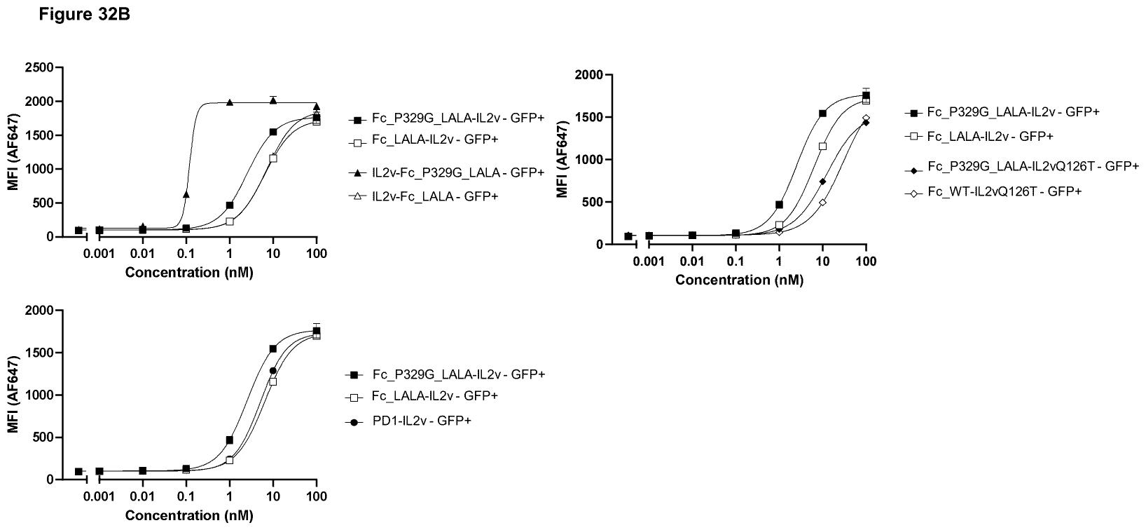

In order to better compare the different engineered T cells, the transduction rate of all transduced cell pools was adjusted to 50% by adding wildtype cells from the same donor, which were activated and cultured in parallel to the transduced cells. The STAT5 phosphorylation assay was performed with the molecules Fc_P329G_LALA-IL2v (SEQ ID NO:42, 43), Fc_LALA-IL2v (SEQ ID NO:42, 45), IL2v-Fc_P329G_LALA (SEQ ID NO:42, 47), IL2v-Fc_LALA (SEQ ID NO:42, 49), Fc_P329G_LALA-IL2vQ126T (SEQ ID NO:42, 44), Fc_WT-IL2vQ126T (SEQ ID NO:42, 46), PD1-IL2v (SEQ ID NOs: 54, 55, 56) and PD1-IL2vQ126T (SEQ ID NOs: 55, 56, 57) at 100 nM, 1:10. The results are shown in FIG. 29 (P329G-CAR), FIG. 30 (P329G-tag, FIG. 31 (P329G-CD38) and FIG. 32 (P329G-Cαβ), FIGS. 29A-32A show the pSTAT5 median fluorescence intensity after gating on eGFP+ or eGFP− cells. The graphs were rearranged to allow a direct comparison of N-terminal vs. C-terminal Fc-IL2v fusion, PD1-IL2v vs. Fc_P329G_LALA-IL2v and Fc-fused IL2v vs. IL2vQ126T (30B-33B).

In all P329G-CAR/TCR formats except for P329G-Cαp, the N-terminal and C-terminal IL2v-Fc fusion showed similar STAT5 phosphorylation. In case of the P329G-Cαβ T cells the N-terminal IL2v fusion showed clearly higher pSTAT5 signaling compared to the C-terminal fusion. IL2v-Fc_P329G_LALA was the only molecule with which preferential cis-targeting to the P329G-Cαβ cells was achieved (FIG. 32A and B). P329G-CAR (CD28) and P329G-tag (IL 15Ra) T cells (PD1 low (FIG. 28C)) incubated with PD1-IL2v showed very similar STAT5 phosphorylation to Fc_P329G_LALA-IL2v, highlighting the targeting of the molecule to the engineered cells via the P329G-mutation irrespective of the PD1 expression. In case of the P329G-TCR cells (FIG. 31B+33B) the smaller Fc-IL2v fusions seem to have a benefit and lead to higher pSTAT5 signaling compared to PD1-IL2v. The further attenuation of IL2v, by inserting the Q126T mutation, lead to an increased cis-targeting window, observed in both molecules (PD1-IL2vQ126T and Fc_P329G_LALA-IL2vQ126T) (e.g. FIGS. 30A and 30B).

These results further highlight the targeting of cytokines, here IL2v or IL2VQ126T, via P329G to the engineered T cells, independent of their PD-1 expression, making a selective expansion of P329G-CAR. P329G-tag, P329G-CD3ε and P329G-Cαβ T cells possible.

### Example 14: Incucyte® Immune Cell Killing Assay with P329G-CD3ε T Cells Expanded with Fc_P329G_LALA-IL2v

T cells were transduced with the P329G-CD3ε (SEQ ID NO:223) to assess if the P329G-CD3ε T cells can be selectively expanded by P329G cis-targeting, before targeting the P329G-TCR with an different molecule (adaptor IgG) for killing. After transduction and knockout the cells were either expanded with advanced RPMI, 10% FBS, 1% Glutamax, 50 IU/Proleukin, 25 ng/ml IL-7 and 50 ng/ml IL-15 (T cell medium) or advanced RPMI, 10% FBS, 1% Glutamax, 0.5 nM Fc_P329G_LALA-IL2v for 6 days. An increase in the desired population from 37% to 87% was achieved (FIG. 33A).

To compare the killing of the differently expanded cell populations, an Incucyte® killing assay was performed (FIG. 33B). In order to compensate for the different P329G-CD3ε expression of the two cell products, the amount of T cells per well was normalized to the percentage of eGFP+ cells (37% versus 87%), and 10,000 eGFP+ T cells/well were seeded to 10,000 target cells (E:T 1:1). The killing assay was performed as described above. Anti-FolR1 IgG P329G LALA (0.1 nM) served as an adaptor IgG for HeLa NLR, while anti-CEACAM5 IgG P329G LALA was used for MKN45 NLR. As negative control for non-targeted killing, the non-specific DP47 IgG P329G LALA (10 nM) or no adaptor IgG were added. The red cell count was analyzed over the course of 5 days. The DP47 adaptor IgG showed no decrease in target cells as expected. In both target cell models the killing observed with the P329G-CD38 T cells expanded with either IL2, IL7, IL15 or Fc_P329G_LALA-IL2v and 0.1 nM adaptor IgG was very similar. Since the same number of effector cells was used, the data presented in FIG. 33B indicates that the P329G-CD3ε receptor is free to bind the adaptor IgG and is not blocked or inhibited by Fc_P329G_LALA-IL2v. It was shown in vitro that a selective expansion of P329G-targeting T cells results in a fully functional T cell product.

### Example 15: STATS Phosphorylation Assay with P329G-Tag (IL15Rα) PD1^{low} T Cells

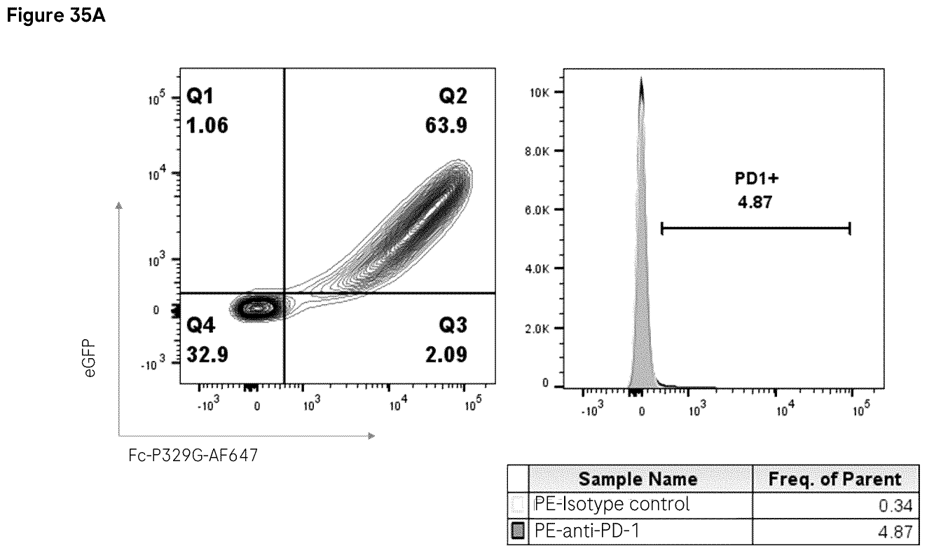

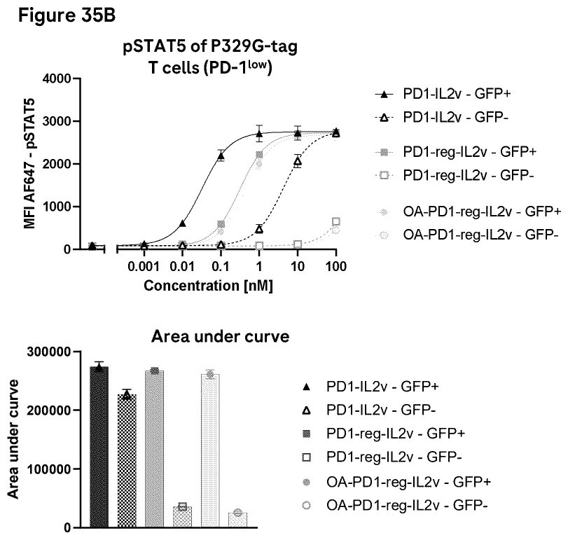

The P329G-tag was transduced into primary T cells as described above. The transduction efficiency was checked and surface expression of the construct was assessed. ˜64% of the cells were successfully transduced (FIG. 35A). PD1 expression was checked briefly before the pSTAT5 assay by staining with PE anti-PD-1 (Biolegend, #329906, 1:100) or PE mouse IgG1, k isotype control (Biolegend, #400113, 1:100). The cells were low in PD-1 expression (˜5%) (FIG. 35A).

The STAT5 phosphorylation assay was performed with the molecules PD1-IL2v (SEQ ID NOs: 54, 55, 56), PD1-reg-IL2v (SEQ ID NOs: 322, 323, 324, 325) and OA-PD1-reg-IL2v (SEQ ID NOs: 326, 327, 328). FIG. 35B shows the results of the three molecules on the transduced GFP+ and the non-transduced GFP-cell population. Shown is the median fluorescence intensity (MFI) of the AF647 anti-Stat5 (pY694) antibody. PD1-IL2v shows an approximately 100-fold cis-targeting window on GFP+ cells vs. GFP− cells. PD1-reg-IL2v has a shift in EC50 compared to PD1-IL2v, but shows almost no activity on GFP-cells. OA-PD1-reg-IL2v shows the same activity as PD1-reg-IL2v. All three molecules can be cis-targeted to P329G-tag+PD-1^{low} T cells via the P329G mutation, independent of the PD-1 expression.

### Example 16: STAT5 Phosphorylation Assay with P329G-Tag (IL15Rα) T Cells-Reactivated (PD-1^{high}) vs not Reactivated (PD-1^{low})

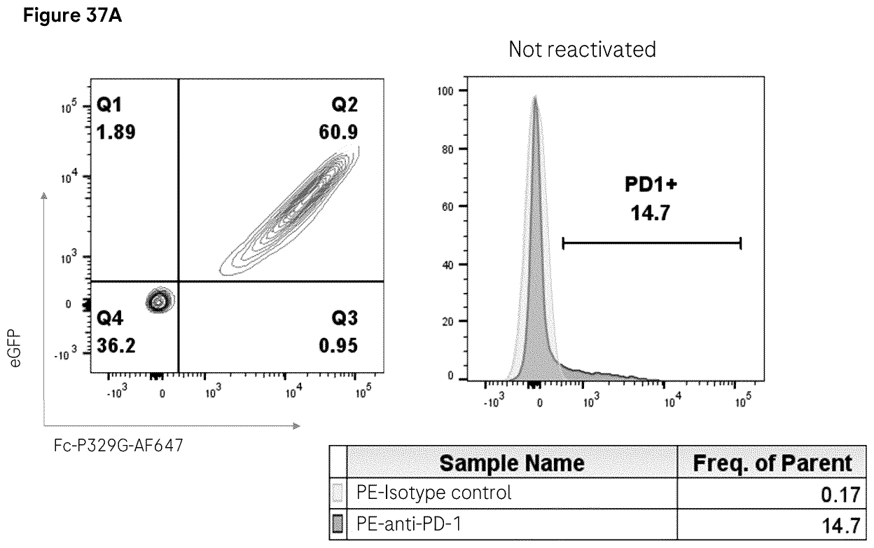

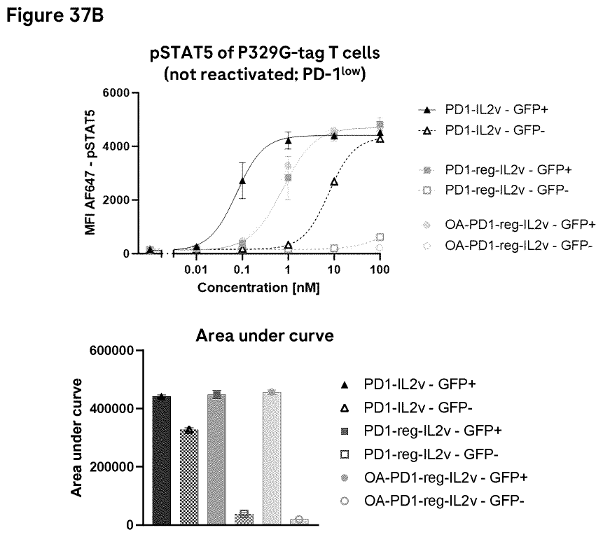

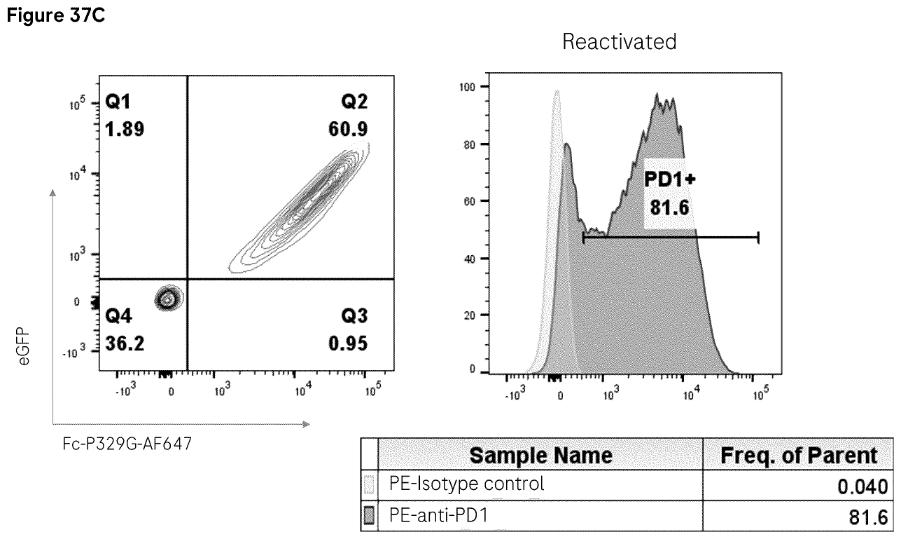

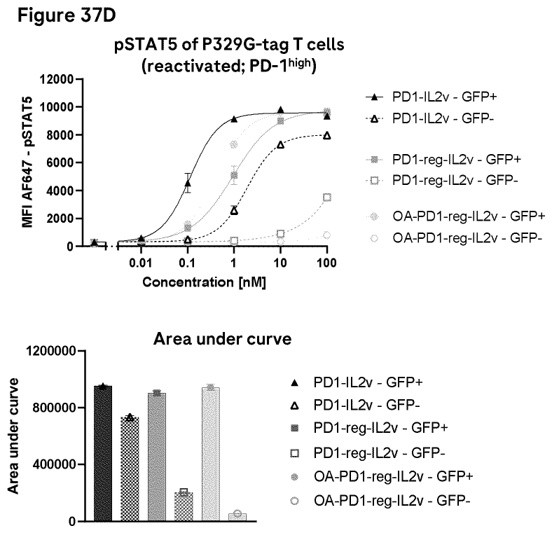

The P329G-tag was transduced into primary T cells as described above. The transduction efficiency was checked and surface expression of the construct was assessed. ˜61% of the cells were successfully transduced (FIG. 37A+C). Some of the cells were reactivated using Dynabeads Human T-Activator CD3/CD28 (ThermoFisher Scientific, #11131D) to increase PD-1 expression on the cell surface. The beads were used according to the manufacturers' protocol and incubated for 24 hours with the T cells. The other fraction of cells was not reactivated to compare the molecules on PD-1^{low} cells.

PD-1 expression was checked briefly before the pSTAT5 assay by staining with PE anti-PD-1 (Biolegend, #329906, 1:100) or PE mouse IgG1, k isotype control (Biolegend, #400113, 1:100). The not reactivated cells were low in PD-1 expression (˜15%) (FIG. 37A), while the reactivated cell fraction showed high expression of PD-1 (˜82%) (FIG. 37C).

The STAT5 phosphorylation assay was performed with the molecules PD1-IL2v (SEQ ID NOs: 54, 55, 56), PD1-reg-IL2v (SEQ ID NOs: 322, 323, 324, 325) and OA-PD1-reg-IL2v (SEQ ID NOs: 326, 327, 328). FIG. 37B shows the results of the three molecules on the transduced GFP+ and the non-transduced GFP-cell population on PD1^{low} cells. Shown is the median fluorescence intensity (MFI) of the AF647 anti-Stat5 (pY694) antibody. The results are comparable to what was seen previously (FIG. 35B). PD1-IL2v shows an approximately 100-fold window on GFP+ cells vs. GFP− cells. PD1-reg-IL2v has a shift in EC50 compared to PD1-IL2v, but shows almost no activity on GFP-cells. OA-PD1-reg-IL2v shows the same activity as PD1-reg-IL2v. All three molecules can be targeted to P329G-tag+PD-1^{low} T cells via the P329G mutation. FIG. 37D shows the results on PD-1^{high} cells. The high PD-1 expression narrows the cis-targeting window to P329G-tag T cells with PD1-IL2v, which is expected, as the molecule gets targeted to P329G-tag negative (GFP−)/PD-1^{high} T cells. The EC50 of PD1-reg-IL2v comes closer to PD1-IL2v, as the unmasking of the IL2v by binding to PD-1 is enhanced. In the PD-1^{high} condition, the OA-PD1-reg-IL2v shows a bigger window compared to PD1-reg-IL2v. This is probably due to the targeting being more guided by the P329G-mutation, as only one PD-1 binding arm is present.

1. A recombinant Fc domain-IL2 variant (Fc-IL2v) polypeptide complex in combination with a recombinant membrane-anchored antigen binding (MAB) polypeptide or MAB polypeptide complex, for use in the treatment of cancer, for use in the prevention or treatment of metastasis, or for use in stimulating an immune response or function, such as T cell activity,
wherein the recombinant Fc-IL2v polypeptide complex comprises:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
(ii) a second polypeptide comprising a variant CH2-CH3 region comprising G329 according to EU numbering, wherein the recombinant Fc-IL2v polypeptide complex does not comprise an antigen binding moiety, and
wherein the MAB polypeptide or MAB polypeptide complex comprises an antigen-binding moiety, or a component thereof, and a transmembrane domain, wherein the antigen-binding moiety binds to a variant CH2-CH3 region comprising the amino acid substitution P329G according to EU numbering relative to the amino acid sequence of a reference CH2-CH3 region comprising P329 according to EU numbering, to which the antigen-binding moiety does not bind.

2. A recombinant Fc domain-IL2 variant (Fc-IL2v) polypeptide complex, comprising:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
(ii) a second polypeptide comprising a variant CH2-CH3 region comprising G329 according to EU numbering, wherein the recombinant Fc-IL2v polypeptide complex does not comprise an antigen binding moiety.

3. A method for treatment or prevention of cancer or for stimulating and immune response or function, such as T cell activity in an individual, wherein said method comprises
(a) administration of a recombinant Fc domain-IL2 variant (Fc-IL2v) polypeptide complex to the individual, wherein the recombinant Fc-IL2v polypeptide complex comprises:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
(ii) a second polypeptide comprising a variant CH2-CH3 region comprising G329 according to EU numbering; and
(b) administration of a recombinant membrane-anchored antigen binding (MAB) polypeptide or MAB polypeptide complex, wherein the MAB polypeptide or MAB polypeptide complex comprises an antigen-binding moiety, or a component thereof, and a transmembrane domain, wherein the antigen-binding moiety binds to a variant CH2-CH3 region comprising the amino acid substitution P329G according to EU numbering, relative to the amino acid sequence of a reference CH2-CH3 region comprising P329 according to EU numbering, to which the antigen-binding moiety does not bind.

4. Use of a recombinant Fc domain-IL2 variant (Fc-IL2v) polypeptide complex in the manufacture of a medicament for treatment or prevention of cancer or for stimulating and immune response or function, such as T cell activity in an individual, wherein the recombinant Fc-IL2v polypeptide complex comprises:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
(ii) a second polypeptide comprising a variant CH2-CH3 region comprising G329 according to EU numbering.

5. Use of a recombinant Fc domain-IL2 variant (Fc-IL2v) polypeptide complex in the manufacture of a medicament for the treatment or prevention of cancer or for stimulating and immune response or function, such as T cell activity in an individual, wherein the treatment comprises:
(a) administration of a recombinant Fc domain-IL2 variant (Fc-IL2v) polypeptide complex to the individual, wherein the recombinant Fc-IL2v polypeptide complex comprises:
(i) a first polypeptide comprising a variant CH2-CH3 region comprising G329 according to EU numbering, and wherein the first polypeptide further comprises an IL-2 variant (IL2v) polypeptide comprising an IL-2 polypeptide comprising the amino acid substitutions F42A, Y45A and L72G wherein numbering is relative to the human IL-2 sequence SEQ ID NO: 40; and
(ii) a second polypeptide comprising a variant CH2-CH3 region comprising G329 according to EU numbering; and
(b) administration of a recombinant membrane-anchored antigen binding (MAB) polypeptide or MAB polypeptide complex, wherein the MAB polypeptide or MAB polypeptide complex comprises an antigen-binding moiety, or a component thereof, and a transmembrane domain, wherein the antigen-binding moiety binds to a variant CH2-CH3 region comprising the amino acid substitution P329G according to EU numbering, relative to the amino acid sequence of a reference CH2-CH3 region comprising P329 according to EU numbering, to which the antigen-binding moiety does not bind.

6. The recombinant Fc-IL2v polypeptide complex, method, or use of any one of the preceding claims, wherein the antigen-binding moiety that binds to the Fc-IL2v comprises the heavy chain variable (VH) region and light chain variable (VL) region of an antibody that binds to the variant CH2-CH3 region comprising the amino acid substitution P329G according to EU numbering.

7. The recombinant Fc-IL2v polypeptide complex, method, or use of any one of the preceding claims, wherein the antigen-binding moiety is or comprises an Fv, scFv, Fab, Fab′, Fab-SH, F(ab′)₂, crossFab, scFab or dAb moiety.

8. The recombinant Fc-IL2v polypeptide complex, method, or use of any one of the preceding claims, wherein the antigen-binding moiety comprises:
(a) (i) a VH region incorporating the following CDRs:
HC-CDR1 having the amino acid sequence of SEQ ID NO: 11:
HC-CDR2 having the amino acid sequence of SEQ ID NO: 19; and
HC-CDR3 having the amino acid sequence of SEQ ID NO: 13;
and
(ii) a VL region incorporating the following CDRs:
LC-CDR1 having the amino acid sequence of SEQ ID NO:24;
LC-CDR2 having the amino acid sequence of SEQ ID NO:25; and
LC-CDR3 having the amino acid sequence of SEQ ID NO:26;
or
(b) (i) a VH region incorporating the following CDRs:
HC-CDR1 having the amino acid sequence of SEQ ID NO:11;
HC-CDR2 having the amino acid sequence of SEQ ID NO: 12; and
HC-CDR3 having the amino acid sequence of SEQ ID NO:13; and
(ii) a VL region incorporating the following CDRs:
LC-CDR1 having the amino acid sequence of SEQ ID NO:24;
LC-CDR2 having the amino acid sequence of SEQ ID NO:25; and
LC-CDR3 having the amino acid sequence of SEQ ID NO:26.

9. The recombinant Fc-IL2v polypeptide complex, method, or use of any one of the preceding claims, wherein the mutant IL-2 polypeptide further comprises the amino acid substitution Q126T.

10. The recombinant Fc-IL2v polypeptide complex, method, or use of any one of the preceding claims, wherein the recombinant Fc-IL2v polypeptide complex does not comprise an antigen binding moiety, in particular wherein the recombinant Fc-IL2v polypeptide complex does not comprise a scFv, Fab or crossFab.

11. The recombinant Fc-IL2v polypeptide complex, method, or use of any one of the preceding claims, wherein the recombinant MAB polypeptide comprises an amino acid sequence derived from IL2Ra, IL15Ra or CD8a.

12. The recombinant Fc-IL2v polypeptide complex, method, or use of any one of the preceding claims, wherein the recombinant MAP polypeptide is a chimeric antigen receptor (CAR).

13. The recombinant Fc-IL2v polypeptide complex, method, or use of any one of the preceding claims, wherein the recombinant MAP polypeptide comprises at least one recombinant CD3-TCR complex polypeptide.

14. The recombinant Fc-IL2v polypeptide complex, method, or use of any one of the preceding claim 13,
wherein the recombinant CD3-TCR complex polypeptide comprises:
(i) an antigen-binding moiety, or a component thereof, wherein the antigen-binding moiety binds to the variant CH2-CH3 region comprising the amino acid substitution P329G according to EU numbering, relative to the amino acid sequence of a reference CH2-CH3 region comprising P329 according to EU numbering, to which the antigen-binding moiety does not bind; and
(ii) a CD3-TCR complex association domain having an amino acid sequence derived from a CD3-TCR complex polypeptide.

15. The recombinant Fc-IL2v polypeptide complex, method, or use claim 14, wherein the recombinant CD3-TCR complex polypeptide is capable of associating through its CD3-TCR complex association domain with one or more CD3-TCR complex polypeptides to form a CD3-TCR complex.

16. The recombinant Fc-IL2v polypeptide complex, method, or use of claim 14 or 15, wherein the amino acid sequence derived from a CD3-TCR complex polypeptide is derived from CD38, TCRα or TCRβ.

17. A cell comprising a recombinant MAB polypeptide or MAB polypeptide complex according to any one of claims 1 or 11 to 16.

18. A method of producing an enriched pool of cells comprising contacting a starting pool of cells comprising at least one cell according to claim 17 with the recombinant Fc-IL2v polypeptide complex of any one of claims 2 or 6 to 10 and incubating the cells until the fraction of cells comprising the recombinant MAB polypeptide or MAB polypeptide complex reaches a desired fraction of the total pool of cells to produce the enriched pool of cells.

19. A nucleic acid, or a plurality of nucleic acids, encoding a recombinant Fc domain-IL2 variant (Fc-IL2v) polypeptide complex according to any one of claims 2 or 6 to 10, or a recombinant MAB polypeptide or MAB polypeptide complex according to any one of claims 1 or 11 to 16.

20. An expression vector, or a plurality of expression vectors, comprising a nucleic acid according to claim 19.

21. A cell comprising a recombinant MAB polypeptide or MAB polypeptide complex according to any one of claims 1 or 11 to 16, a nucleic acid or a plurality of nucleic acids according to claim 19, or an expression vector or a plurality of expression vectors according to claim 20.

22. The recombinant Fc-IL2v polypeptide complex, method, or use of any one of claims 1-16, or the cell of claim 21, wherein cells expressing the recombinant MAB polypeptide and/or the recombinant MAB polypeptide complex cells are specifically expanded, in particular wherein the cells are specifically expanded by contacting the cells with the recombinant Fc-IL2v polypeptide complex of any one of claims 2 or 6 to 10.

23. The recombinant Fc-IL2v polypeptide complex, method, or use of any one of claims 1-16, or the cell of claim 21, wherein cells expressing the recombinant MAB polypeptide and/or the recombinant MAB polypeptide complex cells are enriched, in particular wherein the cells are enriched by contacting the cells with the recombinant Fc-IL2v polypeptide complex of any one of claims 2 or 6 to 10.

24. The recombinant Fc-IL2v polypeptide complex, method, or use of any one of claims 1-16, or the cell of claim 21, wherein cells expressing the recombinant MAB polypeptide and/or the recombinant MAB polypeptide complex cells are enriched to >90% of a total cell pool.

25. A method of producing an enriched pool of cells comprising contacting a starting pool of cells comprising at least one cell according to claim 21 with the recombinant Fc-IL2v polypeptide complex of any one of claims 2 or 6 to 10 and incubating the cells until the fraction of cells comprising the recombinant MAB polypeptide or MAB polypeptide complex reaches a desired fraction of the total pool of cells to produce the enriched pool of cells.

26. A pharmaceutical composition comprising a recombinant Fc domain-IL2 variant (Fc-IL2v) polypeptide complex according to any one of claims 2 or 6 to 10, a cell according to claim 21 or an enriched pool of cells produced according to claim 25.

27. The invention as hereinbefore described with reference to the Figures and Examples.
